(19) United States
(12) Patent Application Publication
(10) Pub. No.: US 2024/0018121 A1
(43) Pub. Date: Jan. 18, 2024

(54) NOVEL HETEROAROMATIC COMPOUNDS EXHIBITING ANTIFUNGAL ACTIVITY AND THEIR METHOD OF USE

(71) Applicant: FOX CHASE CHEMCIAL DIVERSITY CENTER, INC., Doylestown, PA (US)

(72) Inventors: Simon David Peter BAUGH, Ringoes, NJ (US); Kathryn B. FREEMAN, Yardley, PA (US); Jeffrey Claude PELLETIER, Lafayette Hill, PA (US); Allen B. REITZ, Lansdale, PA (US); Richard W. SCOTT, Chadds Ford, PA (US); Garry Robert SMITH, Elverson, PA (US); Sandeep THANNA, Lansdale, PA (US); Damian G. WEAVER, Lansdale, PA (US); David B. WHITMAN, Phoenixville, PA (US)

(73) Assignee: FOX CHASE CHEMCIAL DIVERSITY CENTER, INC., Doylestown, PA (US)

(21) Appl. No.: 18/008,399

(22) PCT Filed: Jun. 3, 2021
(86) PCT No.: PCT/US2021/035568

§ 371 Date: Dec. 5, 2022

Related U.S. Application Data
(60) Provisional application No. 63/035,105, filed on Jun. 5, 2020.

(51) Int. Cl. C07D 401/14; C07D 413/14; C07D 271/107; C07D 231/12; C07D 249/08; A01P 3/00; A61P 31/10; A01N 47/44; C07D 403/14

(52) U.S. Cl. CPC C07D 401/14; C07D 413/14; C07D 271/107; C07D 231/12; C07D 403/14; A01P 3/00; A61P 31/10; A01N 47/44; C07D 249/08

(57) ABSTRACT

Pharmaceutical compositions include heteroaromatic compounds having a disease-modifying action in the treatment of fungal infections and diseases associated with fungal infection.

This application claims benefit of U.S. Provisional Patent Application No. 63/035,105 filed Jun. 5, 2020, the entirety of which is incorporated herein by reference.

### STATEMENT OF FEDERALLY FUNDED RESEARCH

The U.S. Government has a paid-up license in this invention and the right in limited circumstances to require the patent owner to license others on reasonable terms as provided for by the terms of grant number R44AI106270 awarded by the National Institute of Health, and grant number W81XWH1810638 awarded by the US Department of Defense.

### FIELD OF INVENTION

The present invention describes compounds that are antifungal agents, useful for the treatment of, for example, fungal infections and related conditions. The present invention further describes a novel chemotype useful for the treatment of fungal infections and other diseases that involve fungal infection.

### BACKGROUND OF THE INVENTION

Fungal infections are a growing problem in numerous medical settings. Modern medical practices including anticancer chemotherapies, immunosuppressive drugs, broad spectrum antibiotics that disrupt the microbiome and indwelling medical devices that disrupt and breach the protective immune system. This creates an opening for infection by opportunistic fungal pathogens. Fungal infections are most common in immunocompromised patients afflicted with HIV or undergoing cancer therapies, hematological stem cell replacement, or organ transplants. Fungal infections can also occur in immunocompetent individuals and the most common cause is from skin and soft tissue wounds resulting from traumatic injury. Significant morbidity is evident with these types of injuries as the local invasive infections often require frequent and extensive surgical debridement in conjunction with systemic antifungal therapy. Nevertheless, amputations are still needed in many of the cases and mortality can be as high as 25%. In both immunosuppressed and immunocompetent patients, the most common fungal pathogens are Candida, Aspergillus, Cryptococcus, Mucorales and Fusarium spp. and infections are associated with a significant incidence of treatment failure and high mortality. Invasive Candidiasis (IC) is the fourth leading healthcare associated bloodstream infection in the US and is associated with a 47% mortality rate. Invasive Aspergillosis (IA) is becoming a dominant invasive fungal disease in hematological oncology, organ transplant and exacerbated chronic obstructive pulmonary disease. The incidence of IA in hematopoietic stem cell transplants has been reported to be as high as 15% with mortality rates ranging from 20% to 50%.

Pathogenic fungi include the genus Candida (examples include C. albicans, C. glabrata, C. krusei, C. tropicalis, C. guilliermondii, C. parapsilosis, C. dubliniensis and C. auri), the genus Cryptococcus (examples include C. neoformans and C. gatti), the genus Trichosporon (examples include T. asahii, T. asteroides, T. cutaneum, T. dermatis, T. dohaense, T. inkin, T. loubieri, T. mucoides, and T. ovoides), the genus Malassezia (examples include M. globose and M. restricta), the genus Aspergillus (examples include A. fumigatus. A. flavis, A. terreu and A. niger), the genus Fusarium (examples include F. solani, F. falciforme, F. oxysporum, F. verticillioides, and F. proliferatum), the genus Mucor (examples include M. circinelloides, M. ramosissimus, M. indicus, M. rasemosus, and M. piriformis), the genus Blastomyces (examples include B. dermatitidis and B. brasiliensis), the genus Coccidioides (examples include C. immitis, C. and posadasii), the genus Pneumocystis (examples include P. carinii and P. jiroveci), the genus Histoplasma (examples include H. capsulatum), the genus Trichophyton (examples include T. schoenleinii, T. mentagrophytes, T. verrucosum, and T. rubrum), the genus Rhizopus (examples include R. oryzae and R. stolonifera), the genus Apophysomyces (examples include A. variabilis), the genus Rhizomucor (examples include R. pusillus, R. regularior, and R. chlamydosporus), the genus Lichtheimia (examples include L. ramose and L. corymbifera), the genus Scedosporium (examples include S. apiospermum), and the genus Lomentospora (examples include L. prolificans).

The compounds of the disclosure have excellent activity against pathogenic fungi of the genera Candida, Aspergillus, Fusarium, Cryptococcus and Mucor. They can be used to treat fungal diseases, caused by these and other susceptible fungal pathogens, such as Candidemia, Oral Candidiasis, Vulvovaginal Candidiasis (VVC) and Recurrent VVC, Aspergillosis (including Allergic Bronchopulmonary Aspergillosis, Allergic Aspergillus Sinusitis and Invasive and Disseminated Aspergillosis), Cryptococcosis (including Pulmonary Cryptococcosis and Meningeal Cryptococcosis), Mucomycosis, Blastomycosis, Superficial infections (including Skin Keratitis, Athletes Foot, Ringworm, Ocular Keratitis and Onychomycosis) and other Invasive infections (including Sinusitis, Endophthalmitis, Otitis, Endocarditis, Pneumonia, Osteomyelitis, Meningitis and Ventriculitis).

Compounds of the disclosure can also be used to treat fungal infections in agricultural crops including Wilt disease in tomato and cotton caused by Fusarium oxysporus, Wilt of Gram caused by Fusarium orthacereas, Downy Mildew of cereals caused by Sclerospora graminicola, Damping of Seedling caused by Phythium spp., Rot of Ginger caused by Phythium deharyaum, Late Blight of Potato caused by Phytophthora infestans, Early Blight of Potato caused by Alternaria solani, Blast Disease of Rice caused by Phyricularia oryzae, Powdery Mildews caused by Erysiphe spp., Tikka Disease of Groundnut caused by Cerecospora personata, Haemelia vastatrix and Cellectotrichum falcatum, Brown Rot in Pear, Plum and Peach caused by Sclerotinia fruiticola, Leaf Spot of Oats caused by Helminthosporium avenae, Leaf Rust of Coffee caused by Haemelia vastatrix, Red Rot of Sugarcane caused by Collectotrichum falcatum, Black Wart Disease of Potato caused by Synchytrium endobioticum, Yellow Rust of Wheat caused by Puccinia striiformis, Maize Smut caused by Ustilago maydis, Loose Smut of Wheat caused by Ustilago tritici, Covered Smut of Oat caused by Ustilago avenae, Flag Smut of Wheat caused by Urocystis tritici, Covered Smut of Barley caused by Ustilago hordei, Black Rust of Wheat caused by Puccinia graminis tritici, Bankanese Disease and Foot Rot of Rice caused by Gibberealla fujikuri, and Ergot Disease of Rye caused by Claviceps purpurea.

Compounds of the disclosure can also be used to treat or prevent fungal infections in domesticated animals, livestock, and companion animals including candidiasis infections in animals selected from the group consisting of cattle, sheep, pigs, goats, horses, donkeys, mules, buffalo, oxen, llamas, camels, dogs, cats, horses, rabbits, ferrets, and guinea pigs.

Compounds of the disclosure can also be used to treat or prevent diseases or conditions associated with fungal infection in domesticated animals, livestock, and companion animals such as cattle, sheep, pigs, goats, horses, donkeys, mules, buffalo, oxen, llamas, camels, dogs, cats, horses, rabbits, ferrets, and guinea pigs, wherein said diseases or conditions is selected from the group consisting of keratitis, arthritis, endocarditis, disseminated, mastitis, otitis externa, peritonitis, dermatitis, pneumoniagranulomatous rhinitis, intestinal granuloma, and pyothorax.

Compounds of the disclosure can also be used to treat or prevent aspergillosis infections in horses, cattle, sheep, goats, dogs and cats.

Compounds of the disclosure can also be used to treat or prevent diseases or conditions associated with aspergillosis infections in horses, cattle, sheep, goats, dogs and cats including diseases or conditions such as guttural pouch, keratomycosis, pneumonia, mycotic pneumonia, gastroenteritis, mastitis, and placentitis.

Compounds of the disclosure can also be used to treat or prevent mucormycosis infections in horses, cattle, sheep, goats, dogs and cats.

Compounds of the disclosure can also be used to treat or prevent diseases or conditions associated with mucormycosis infections in horses, cattle, sheep, goats, dogs and cats including diseases or conditions such as mucormycotic ruminitis, lymphadentitis, and enteritis.

Compounds of the disclosure can also be used to treat or prevent coccidioidomycosis in dogs and cats caused by infection with an organism selected from the group consisting of Coccidioides immitis and Coccidioides posadasii.

Compounds of the disclosure can also be used to treat or prevent blastomycosis in dogs and cats caused by infection with Blastomyces dermatitidis.

Compounds of the disclosure can also be used to treat or prevent Paracoccidioidomycosis in dogs caused by infection with Paracoccidioides brasiliensis,

Compounds of the disclosure can also be used to treat or prevent dermatophytosis (ringworm) in cats and dogs caused by infection with an organism selected from the group consisting of Microsporum canis, Microsporum gypseum, and Trichophyton mentagrophytes

Compounds of the disclosure can also be used to treat or prevent cryptococcosis in dogs and cats caused by infection with an organism selected from the group consisting of Cryptococcus neoformans and Cryptococcus gattii,

Compounds of the disclosure can also be used to treat or prevent histoplasmosis in dogs caused by infection with Histoplasma capsulatum.

### BRIEF SUMMARY OF THE INVENTION

The present invention is directed toward novel heteroaromatic compounds of formula (I),

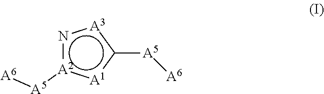

Including enantiomers, diastereomers, hydrates, solvates, pharmaceutically acceptable salts, prodrugs and complexes thereof, wherein:
A¹ is selected from the group consisting of CR¹, O, N, and NR¹;
When A¹ is CR¹, A², A³ are N;
Alternately, when A¹ is CR¹, A² is C, and A³ is NR^{1a};
When A¹ is O, A² is C, and A³ is N;
When A¹ is NR¹, A² is C, and A³ is N;
When A¹ is N, A² is C, and A³ is NR^{1a};
A⁵ is at each occurrence independently selected from the group consisting of

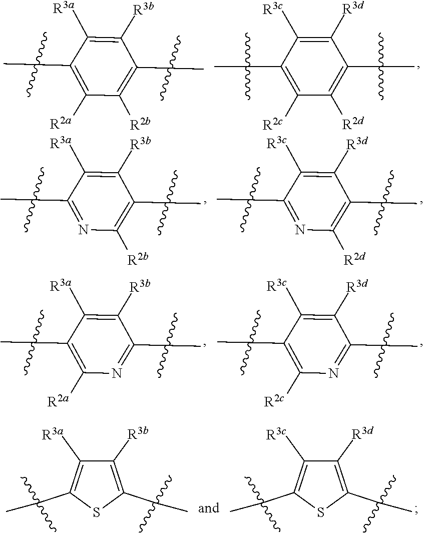

A⁶ is at each occurrence independently selected from the group consisting of,

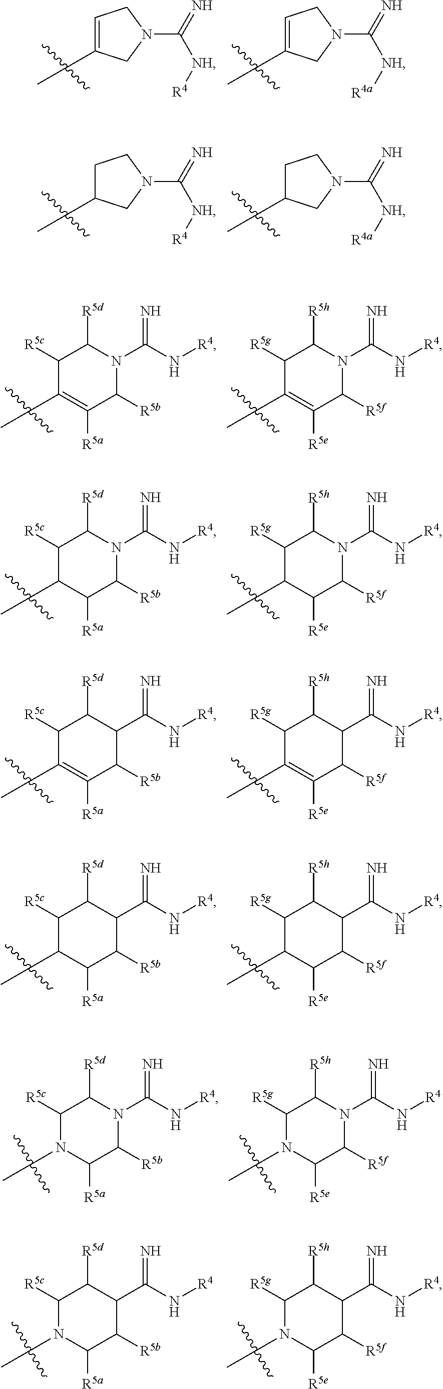

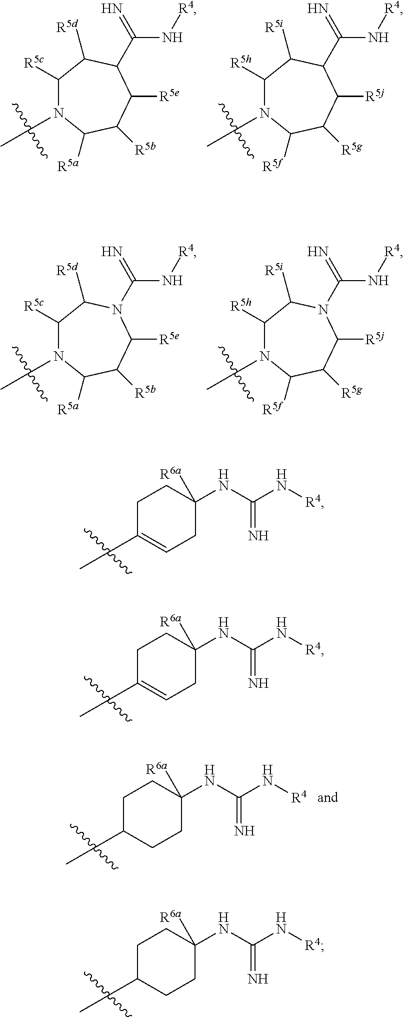

R¹ is selected from the group consisting of hydrogen C₁₋₈ alkyl, C₃₋₈ branched alkyl, C₃₋₈ cycloalkyl, optionally substituted benzyl,

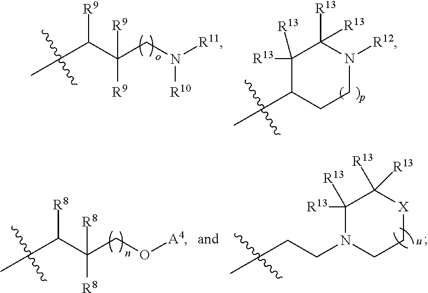

R^{1a} is selected from the group consisting of hydrogen, C₁₋₈ alkyl, C₃₋₈ branched alkyl, C₃₋₈ cycloalkyl, optionally substituted benzyl,

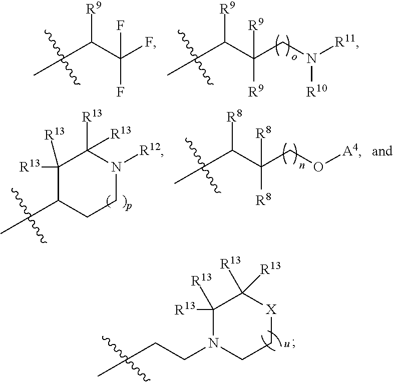

A⁴ is selected from the group consisting of hydrogen, C₁₋₄ alkyl, C₃₋₅ branched alkyl, C₃₋₈ cycloalkyl,

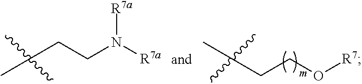

R⁷ is selected from the group consisting of hydrogen, C₁₋₄ alkyl, and C₃₋₅ branched alkyl;
R^{7a} is selected from the group consisting of C₁₋₄ alkyl, C₃₋₈ branched alkyl, and C₃₋₈ cycloalkyl;
R⁸ is at each occurrence independently selected from the group consisting of hydrogen and C₁₋₄alkyl;
R^{8a} is selected from the group consisting of hydrogen, C₁₋₄ alkyl, and

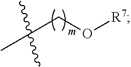

R⁹ is at each occurrence independently selected from the group consisting of hydrogen, and C₁₋₄ alkyl;
R¹⁰ is selected from the group consisting of hydrogen, C₁₋₄ alkyl, C₃₋₈ branched alkyl, and C₃₋₈ cycloalkyl;
R¹¹ is selected from the group consisting of C₁₋₄ alkyl, C₃₋₈ branched alkyl, C₃₋₈ cycloalkyl, and

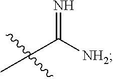 [SMILES: *C(=N)N]

In some embodiments R¹⁰ and R¹¹ are optionally joined to form a heterocyclic ring consisting of three, four, five, six, or seven members;
R¹² is selected from the group consisting of C₁₋₄ alkyl, C₃₋₈ branched alkyl, C₃₋₈ cycloalkyl, and

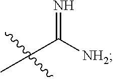 [SMILES: *C(=N)N]

R¹³ is at each occurrence independently selected from the group consisting of hydrogen and C₁₋₄ alkyl;
p is 0, 1, or 2;
o is 0, 1, or 2;
n is 0, 1, or 2;
m is 1, 2, or 3;
u is 1 or 2;
X is selected from the group consisting of NR¹², oxygen, sulfur, and SO₂;
R^{2a} is selected from the group consisting of hydrogen, C₁₋₄alkyl, fluorine, chlorine, C₁₋₄-alkoxy, CN, CF₃, and OCF₃;
R^{2b} is selected from the group consisting of hydrogen, C₁₋₄ alkyl, fluorine, chlorine, C₁₋₄-alkoxy, CN, CF₃, and OCF₃;
R^{2c} is selected from the group consisting of hydrogen, C₁₋₄ alkyl, fluorine, chlorine, C₁₋₄-alkoxy, CN, CF₃, and OCF₃;
R^{2d} is selected from the group consisting of hydrogen, C₁₋₄ alkyl, fluorine, chlorine, C₁₋₄-alkoxy, CN, CF₃, and OCF₃;
R^{3a} is selected from the group consisting of hydrogen, C₁₋₄ alkyl, fluorine, chlorine, C₁₋₄-alkoxy, CN, CF₃, and OCF₃;
R^{3b} is selected from the group consisting of hydrogen, C₁₋₄ alkyl, fluorine, chlorine, C₁₋₄-alkoxy, CN, CF₃, and OCF₃;
R^{3c} is selected from the group consisting of hydrogen, C₁₋₄alkyl, fluorine, chlorine, C₁₋₄-alkoxy, CN, CF₃, and OCF₃;
R^{3d} is selected from the group consisting of hydrogen, C₁₋₄ alkyl, fluorine, chlorine, C₁₋₄-alkoxy, CN, CF₃, and OCF₃;
R⁴ is selected from the group consisting of hydrogen and C₁₋₄ alkyl;
R^{4a} is selected from the group consisting of hydrogen and C₁₋₄ alkyl;
R^{5a} is selected from the group consisting of hydrogen and C₁₋₄ alkyl;
R^{5b} is selected from the group consisting of hydrogen and C₁₋₄ alkyl;
R^{5c} is selected from the group consisting of hydrogen and C₁₋₄ alkyl;
R^{5d} is selected from the group consisting of hydrogen and C₁₋₄ alkyl;
R^{5e} is selected from the group consisting of hydrogen and C₁₋₄ alkyl;
R^{5f} is selected from the group consisting of hydrogen and C₁₋₄ alkyl;
R^{5g} is selected from the group consisting of hydrogen and C₁₋₄ alkyl;
R^{hh} is selected from the group consisting of hydrogen and C₁₋₄ alkyl;
R⁵ⁱ is selected from the group consisting of hydrogen and C₁₋₄ alkyl;
R^{5j} is selected from the group consisting of hydrogen and C₁₋₄ alkyl;
R^{6a} is selected from the group consisting of hydrogen and C₁₋₄ alkyl;
R^{6b} is selected from the group consisting of hydrogen and C₁₋₄ alkyl.

The compounds of the present invention include compounds having formula (II):

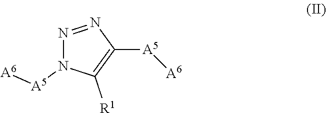

including enantiomers, diastereomers, hydrates, solvates, pharmaceutically acceptable salts, and complexes thereof.

The compounds of the present invention include compounds having formula (III):

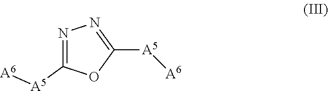

including enantiomers, diastereomers, hydrates, solvates, pharmaceutically acceptable salts, and complexes thereof.

The compounds of the present invention include compounds having formula (IV):

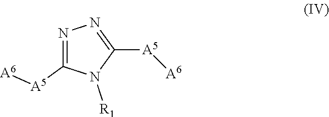

including enantiomers, diastereomers, hydrates, solvates, pharmaceutically acceptable salts, and complexes thereof.

The compounds of the present invention include compounds having formula (V):

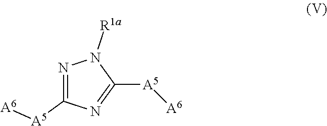

including enantiomers, diastereomers, hydrates, solvates, pharmaceutically acceptable salts, and complexes thereof.

The compounds of the present invention include compounds having formula (VI):

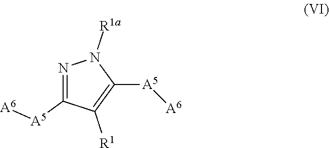

including enantiomers, diastereomers, hydrates, solvates, pharmaceutically acceptable salts, and complexes thereof.

The compounds of the present invention include compounds having formula (VII):

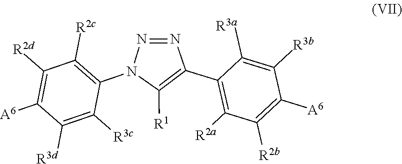

including enantiomers, diastereomers, hydrates, solvates, pharmaceutically acceptable salts, and complexes thereof.

The compounds of the present invention include compounds having formula (VIII):

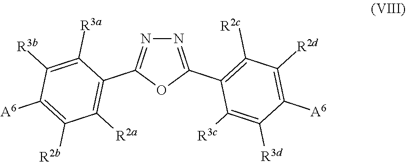

including enantiomers, diastereomers, hydrates, solvates, pharmaceutically acceptable salts, and complexes thereof.

The compounds of the present invention include compounds having formula (IX):

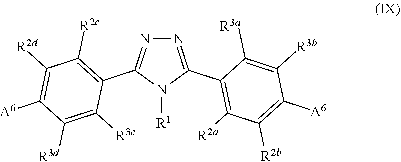

including enantiomers, diastereomers, hydrates, solvates, pharmaceutically acceptable salts, and complexes thereof.

The compounds of the present invention include compounds having formula (X):

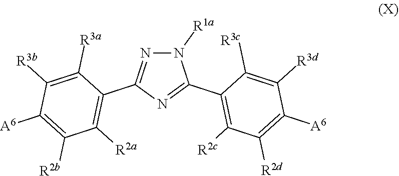

including enantiomers, diastereomers, hydrates, solvates, pharmaceutically acceptable salts, and complexes thereof.

The compounds of the present invention include compounds having formula (XI):

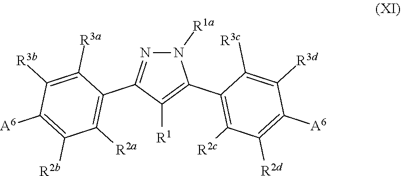

including enantiomers, diastereomers, hydrates, solvates, pharmaceutically acceptable salts, and complexes thereof.

The present invention further relates to compositions comprising: an effective amount of one or more compounds according to the present invention and an excipient.

The present invention also relates to a method for treating or preventing disease or conditions associated with fungal infection. Said methods comprise administering to a subject an effective amount of a compound or composition according to the present invention.

The present invention yet further relates to a method for treating or preventing disease or conditions associated with fungal infection, wherein said method comprises selecting a subject in need of treating or preventing disease or conditions associated with fungal infections and administering to the subject a composition comprising an effective amount of one or more compounds according to the present invention and an excipient, thereby treating or preventing disease or conditions associated with fungal infection in the subject.

The present invention also relates to a method for treating or preventing fungal infection, including, for example, infection with an organism from a genus selected from the group consisting of Candida, Cryptococcus, Trichosporon, Malassezia, Aspergillus, Fusarium, Mucor, Blastomyces, Coccidioides, Pneumocystis, Histoplasma, Trichophyton, Rhizopus, Apophysomyces, Rhizomucor, Lichtheimia, Scedosporium, and Lomentospora, said method comprising selecting a subject in need of treating or preventing disease or conditions associated with fungal infection as set forth herein and administering to the subject an effective amount of a compound or composition according to the present invention, thereby treating or preventing disease or conditions associated with fungal infection in the subject.

The present invention yet further relates to a method for treating or preventing fungal infection, including, for example, infection with an organism from a genus selected from the group consisting of Candida, Cryptococcus, Trichosporon, Malassezia, Aspergillus, Fusarium, Mucor, Blastomyces, Coccidioides, Pneumocystis, Histoplasma, Trichophyton, Rhizopus, Apophysomyces, Rhizomucor, Lichtheimia, Scedosporium, and Lomentospora, wherein said method comprises selecting a subject in need of treating or preventing disease or conditions associated with fungal infections and administering to a subject a composition comprising an effective amount of one or more compounds according to the present invention and an excipient, thereby treating or preventing disease or conditions associated with fungal infection in the subject.

The present invention also relates to a method for treating or preventing fungal infection, including, for example, infection with an organism such as Candida albicans, Candida glabrata, Candida krusei, Candida tropicalis, Candida guilliermondii, Candida parapsilosis, Candida dubliniensis Candida auris, Cryptococcus neoformans, Cryptococcus gatti, Trichosporon asahii, Trichosporon asteroides, Trichosporon cutaneum, Trichosporon dermatis, Trichosporon dohaense, Trichosporon inkin, Trichosporon loubieri, Trichosporon mucoides, Trichosporon ovoides, Malassezia globose, Malassezia restricta, Aspergillus fumigatus, Aspergillus, Aryls, Aspergillus terreus, Aspergillus niger, Fusarium solani, Fusarium falciforme, Fusarium oxysporum, Fusarium verticillioides, Fusarium proliferatum, Mucor circinelloides, Mucor ramosissimus, Mucor indices, Mucor rasemosus, Mucor piriformis, Blastomyces dermatitidis, Blastomyces brasiliensis, Coccidioides immitis, Coccidioides posadasii, Pneumocystis carinii, Pneumocystis jiroveci, Histoplasma capsulatum, Trichophyton schoenleinii, Trichophyton mentagrophytes, Trichophyton verrucosum, Trichophyton rubrum, Rhizopus oryzae, Rhizopus stolonifera, Apophysomyces variabilis, Rhizomucor pusillus, Rhizomucor regularior, Rhizomucor chlamydosporus, Lichtheimia ramosa, Lichtheimia corymbifera, Scedosporium apiospermum, and Lomentospora prolificans, said method comprising administering to a subject an effective amount of a compound or composition according to the present invention, thereby treating or preventing fungal infection in the subject.

The present invention yet further relates to a method for treating or preventing fungal infection, including, for example, infection with an organism such as Candida albicans, Candida glabrata, Candida krusei, Candida tropicalis, Candida guilliermondii, Candida parapsilosis, Candida dubliniensis Candida auris, Cryptococcus neoformans, Cryptococcus gatti, Trichosporon asahii, Trichosporon asteroides, Trichosporon cutaneum, Trichosporon dermatis, Trichosporon dohaense, Trichosporon inkin, Trichosporon loubieri, Trichosporon mucoides, Trichosporon ovoides, Malassezia globose, Malassezia restricta, Aspergillus fumigatus, Aspergillus flavis, Aspergillus terreus, Aspergillus niger, Fusarium solani, Fusarium falciforme, Fusarium oxysporum, Fusarium verticillioides, Fusarium proliferatum, Mucor circinelloides, Mucor ramosissimus, Mucor indices, Mucor rasemosus, Mucor piriformis, Blastomyces dermatitidis, Blastomyces brasiliensis, Coccidioides immitis, Coccidioides posadasii, Pneumocystis carinii, Pneumocystis jiroveci, Histoplasma capsulatum, Trichophyton schoenleinii, Trichophyton mentagrophytes, Trichophyton verrucosum, Trichophyton rubrum, Rhizopus oryzae, Rhizopus stolonifera, Apophysomyces variabilis, Rhizomucor pusillus, Rhizomucor regularior, Rhizomucor chlamydosporus, Lichtheimia ramosa, Lichtheimia corymbifera, Scedosporium apiospermum, and Lomentospora prolificans, wherein said method comprises administering to a subject a composition comprising an effective amount of one or more compounds according to the present invention and an excipient, thereby treating or preventing fungal infection in the subject.

The present invention also relates to a method for treating or preventing disease or conditions associated with fungal infection, including candidemia, oral candidiasis, vulvovaginal candidiasis, aspergillosis, allergic bronchopulmonary aspergillosis, allergic Aspergillus sinusitis, invasive aspergillosis, disseminated aspergillosis, cryptococcosis, pulmonary cryptococcosis, meningeal cryptococcosis, skin keratitis, athlete's foot, ringworm, ocular keratitis, onychomycosis, sinusitis, endophthalmitis, otitis, endocarditism pneumonia, osteomyelitis, meningitis, and ventriculitis. Said methods comprise selecting a subject in need of treating or preventing disease or conditions associated with fungal infections, and administering to the subject an effective amount of a compound or composition according to the present invention, thereby treating or preventing disease or conditions associated with fungal infection in the subject.

The present invention yet further relates to a method for treating or preventing disease or conditions associated with fungal infection, including candidemia, oral candidiasis, vulvovaginal candidiasis, aspergillosis, allergic bronchopulmonary aspergillosis, allergic Aspergillus sinusitis, invasive aspergillosis, disseminated aspergillosis, cryptococcosis, pulmonary cryptococcosis, meningeal cryptococcosis, skin keratitis, athlete's foot, ringworm, ocular keratitis, onychomycosis, sinusitis, endophthalmitis, otitis, endocarditism pneumonia, osteomyelitis, meningitis, and ventriculitis, wherein said method comprises selecting a subject in need of treating or preventing disease or conditions associated with fungal infections, and administering to a subject a composition comprising an effective amount of one or more compounds according to the present invention and an excipient, thereby treating or preventing disease or conditions associated with fungal infection in the subject.

The present invention also relates to a method for treating or preventing diseases or conditions associated with fungal infection, including infection with an organism from a genus selected from the groups consisting of Candida, Cryptococcus, Trichosporon, Malassezia, Aspergillus, Fusarium, Mucor, Blastomyces, Coccidioides, Pneumocystis, Histoplasma, Trichophyton, Rhizopus, Apophysomyces, Rhizomucor, Lichtheimia, Scedosporium, and Lomentospora. Said methods comprise selecting a subject in need of treating or preventing disease or conditions associated with fungal infection and administering to a subject an effective amount of a compound or composition according to the present invention, thereby treating or preventing disease or conditions associated with fungal infection in the subject.

The present invention also relates to a method for treating or preventing diseases or conditions associated with fungal infection, including infection with an organism from a genus selected from the groups consisting of Candida, Cryptococcus, Trichosporon, Malassezia, Aspergillus, Fusarium, Mucor, Blastomyces, Coccidioides, Pneumocystis, Histoplasma, Trichophyton, Rhizopus, Apophysomyces, Rhizomucor, Lichtheimia, Scedosporium, and Lomentospora, wherein said method comprises selecting a subject in need of treating or preventing disease or conditions associated with fungal infection and administering to the subject a composition comprising an effective amount of one or more compounds according to the present invention and an excipient, thereby treating or preventing disease or conditions associated with fungal infection in the subject.

The present invention also relates to a method for treating or preventing diseases or conditions associated with fungal infection, including infection with an organism selected from the groups consisting of Candida albicans, Candida glabrata, Candida krusei, Candida tropicalis, Candida guilliermondii, Candida parapsilosis, Candida dubliniensis Candida auris, Cryptococcus neoformans, Cryptococcus gatti, Trichosporon asahii, Trichosporon asteroides, Trichosporon cutaneum, Trichosporon dermatis, Trichosporon dohaense, Trichosporon inkin, Trichosporon loubieri, Trichosporon mucoides, Trichosporon ovoides, Malassezia globose, Malassezia restricta, Aspergillus fumigatus, Aspergillus, flavis, Aspergillus terreus, Aspergillus niger, Fusarium solani, Fusarium falciforme, Fusarium oxysporum, Fusarium verticillioides, Fusarium proliferatum, Mucor circinelloides, Mucor ramosissimus, Mucor indices, Mucor rasemosus, Mucor piriformis, Blastomyces dermatitidis, Blastomyces brasiliensis, Coccidioides immitis, Coccidioides posadasii, Pneumocystis carinii, Pneumocystis jiroveci, Histoplasma capsulatum, Trichophyton schoenleinii, Trichophyton mentagrophytes, Trichophyton verrucosum, Trichophyton rubrum, Rhizopus oryzae, Rhizopus stolonifera, Apophysomyces variabilis, Rhizomucor pusillus, Rhizomucor regularior, Rhizomucor chlamydosporus, Lichtheimia ramosa, Lichtheimia corymbifera, Scedosporium apiospermum, and Lomentospora prolificans, said method comprising selecting a subject in need of treating or preventing disease or conditions associated with fungal infection and administering to a subject an effective amount of a compound or composition according to the present invention, thereby treating or preventing disease or conditions associated with fungal infection in the subject.

The present invention also relates to a method for treating or preventing diseases or conditions associated with fungal infection, including infection with an organism selected from the groups consisting Candida albicans, Candida glabrata, Candida krusei, Candida tropicalis, Candida guilliermondii, Candida parapsilosis, Candida dubliniensis Candida auris, Cryptococcus neoformans, Cryptococcus gatti, Trichosporon asahii, Trichosporon asteroides, Trichosporon cutaneum, Trichosporon dermatis, Trichosporon dohaense, Trichosporon inkin, Trichosporon loubieri, Trichosporon mucoides, Trichosporon ovoides, Malassezia globose, Malassezia restricta, Aspergillus fumigatus, Aspergillus flavis, Aspergillus terreus, Aspergillus niger, Fusarium solani, Fusarium falciforme, Fusarium oxysporum, Fusarium verticillioides, Fusarium proliferatum, Mucor circinelloides, Mucor ramosissimus, Mucor indices, Mucor rasemosus, Mucor piriformis, Blastomyces dermatitidis, Blastomyces brasiliensis, Coccidioides immitis, Coccidioides posadasii, Pneumocystis caring, Pneumocystis jiroveci, Histoplasma capsulatum, Trichophyton schoenleinii, Trichophyton mentagrophytes, Trichophyton verrucosum, Trichophyton rubrum, Rhizopus oryzae, Rhizopus stolonifera, Apophysomyces variabilis, Rhizomucor pusillus, Rhizomucor regularior, Rhizomucor chlamydosporus, Lichtheimia ramosa, Lichtheimia corymbifera, Scedosporium apiospermum, and Lomentospora prolificans, wherein said method comprises selecting a subject in need of treating or preventing disease or conditions associated with fungal infection and administering to the subject a composition comprising an effective amount of one or more compounds according to the present invention and an excipient, thereby treating or preventing disease or conditions associated with fungal infection in the subject.

The present invention also relates to a method for treating or preventing fungal infection in plants including wilt disease in tomato, wilt disease cotton, wilt disease banana, wilt of gram, downy mildew of cereals, damping of seedling, rot of ginger, late blight of potato, early blight of potato, blast disease of rice, powdery mildews, tikka disease of groundnut, leaf rust of coffee, red rot of sugarcane, brown rot in pear, brown rot in plum, brown rot in peach, leaf spot of oats, black wart disease of potato, yellow rust of wheat, white rust of crucifers, maize smut, loose smut of wheat, flag smut of wheat, covered smut of barley, black rust of wheat, bankanese disease foot rot of rice, and ergot disease of rye. Said methods comprise selecting a plant in need of treating or preventing disease or conditions associated with fungal infection and administering to a plant an effective amount of a compound or composition according to the present invention, thereby treating or preventing said fungal infection in the plant.

The present invention also relates to a method for treating or preventing fungal infection in plants including wilt disease in tomato, wilt disease cotton, wilt disease banana, wilt of gram, downy mildew of cereals, damping of seedling, rot of ginger, late blight of potato, early blight of potato, blast disease of rice, powdery mildews, tikka disease of groundnut, leaf rust of coffee, red rot of sugarcane, brown rot in pear, brown rot in plum, brown rot in peach, leaf spot of oats, black wart disease of potato, yellow rust of wheat, white rust of crucifers, maize smut, loose smut of wheat, flag smut of wheat, covered smut of barley, black rust of wheat, bankanese disease, foot rot of rice, and ergot disease of rye wherein said method comprises selecting a plant in need of treating or preventing said fungal infection, and administering to a plant a composition comprising an effective amount of one or more compounds according to the present invention and an excipient, thereby treating or preventing said fungal infection in the plant.

The present invention also relates to a method for treating or preventing fungal infections in domesticated animals, livestock, and companion animals including candidiasis infections in animals selected from the group consisting of cattle, sheep, pigs, goats, horses, donkeys, mules, buffalo, oxen, llamas, camels, dogs, cats, rabbits, ferrets, and guinea pigs, said method comprising selecting said domesticated animal, livestock, or companion animal in need of treating or preventing fungal infection, and administering to a subject an effective amount of a compound or composition according to the present invention, thereby treating or preventing fungal infections in the domesticated animals, livestock, or companion animals.

The present invention also relates to a method for treating or preventing fungal infections in domesticated animals, livestock, and companion animals including candidiasis infections in animals selected from the group consisting of cattle, sheep, pigs, goats, horses, donkeys, mules, buffalo, oxen, llamas, camels, dogs, cats, rabbits, ferrets, and guinea pigs, said wherein said method comprises selecting said domesticated animal, livestock, or companion animal in need of treating or preventing fungal infection, and administering to a subject a composition comprising an effective amount of one or more compounds according to the present invention and an excipient, thereby treating or preventing fungal infections in the domesticated animals, livestock, or companion animals.

The present invention also relates to a method for treating or preventing diseases or conditions associated with fungal infection in domesticated animals, livestock, and companion animals such as cattle, sheep, pigs, goats, horses, donkeys, mules, buffalo, oxen, llamas, camels, dogs, cats, rabbits, ferrets, and guinea pigs, wherein said diseases or conditions associated with fungal infection is selected from the group consisting of keratitis, arthritis, endocarditis, disseminated, mastitis, otitis externa, peritonitis, dermatitis, otitis externa, keratitis, pneumoniagranulomatous rhinitis, intestinal granuloma, and pyothorax, wherein said method comprising selecting said domesticated animal, livestock, or companion animal in need of treating or preventing diseases or conditions associated with fungal infection, and administering to a subject an effective amount of a compound or composition according to the present invention, thereby treating or preventing diseases or conditions associated with fungal infections in the domesticated animals, livestock, or companion animals.

The present invention also relates to a method for treating or preventing diseases or conditions associated with fungal infection in domesticated animals, livestock, and companion animals such as cattle, sheep, pigs, goats, horses, donkeys, mules, buffalo, oxen, llamas, camels, dogs, cats, rabbits, ferrets, and guinea pigs, wherein said diseases or conditions associated with fungal infection is selected from the group consisting of keratitis, arthritis, endocarditis, disseminated, mastitis, otitis externa, peritonitis, dermatitis, otitis externa, keratitis, pneumoniagranulomatous rhinitis, intestinal granuloma, and pyothorax, wherein said method comprises selecting domesticated animals, livestock, and companion animals in need of treating or preventing disease or conditions associated with said fungal infection administering to a subject a composition comprising an effective amount of one or more compounds according to the present invention and an excipient, thereby treating or preventing disease or conditions associated with said fungal infection in the domesticated animals, livestock, and companion animals.

The present invention also relates to a method for treating or preventing aspergillosis infections in horses, cattle, sheep, goats, dogs and cats said method comprising selecting a subject for treating or preventing aspergillosis infections, and administering to a subject an effective amount of a compound or composition according to the present invention, thereby treating or preventing aspergillosis infections in the subject.

The present invention also relates to a method for treating or preventing aspergillosis infections in horses, cattle, sheep, goats, dogs and cats wherein said method comprises selecting a subject for treating or preventing aspergillosis infections and administering to a subject a composition comprising an effective amount of one or more compounds according to the present invention and an excipient, thereby treating or preventing aspergillosis infections in the subject.

The present invention also relates to a method for treating or preventing diseases or conditions associated with aspergillosis infections in horses, cattle, sheep, goats, dogs and cats, including diseases or conditions such as guttural pouch, keratomycosis, pneumonia, mycotic pneumonia, gastroenteritis, mastitis, and placentitis, said method comprising selecting a subject for treating or preventing aspergillosis infections and administering to the subject an effective amount of a compound or composition according to the present invention, thereby treating or preventing aspergillosis infections in the subject.

The present invention also relates to a method for treating or preventing diseases or conditions associated with aspergillosis infections in horses, cattle, sheep, goats, dogs and cats including diseases or conditions such as guttural pouch, keratomycosis, pneumonia, mycotic pneumonia, gastroenteritis, mastitis, and placentitis, wherein said method comprises selecting a subject in need of treating or preventing aspergillosis infections and administering to a subject a composition comprising an effective amount of one or more compounds according to the present invention and an excipient, thereby treating or preventing aspergillosis infections in the subject.

The present invention also relates to a method for treating or preventing mucormycosis infections in horses, cattle, sheep, goats, dogs and cats said method comprising selecting a subject in need of treating or preventing mucormycosis infections and administering to the subject an effective amount of a compound or composition according to the present invention, thereby treating or preventing mucormycosis infections in the subject.

The present invention also relates to a method for treating or preventing mucormycosis infections in horses, cattle, sheep, goats, dogs and cats, wherein said method comprises selecting a subject in need of treating or preventing mucormycosis infections and administering to a subject a composition comprising an effective amount of one or more compounds according to the present invention and an excipient, thereby treating or preventing mucormycosis infections in the subject.

The present invention also relates to a method for treating or preventing diseases or conditions associated with mucormycosis infections in horses, cattle, sheep, goats, dogs and cats including diseases or conditions such as mucormycotic ruminitis, lymphadentitis, and enteritis, said method comprising selecting a subject in need of treating or preventing mucormycosis infections and administering to the subject an effective amount of a compound or composition according to the present invention, thereby treating or preventing mucormycosis infections in the subject.

The present invention also relates to a method for treating or preventing diseases or conditions associated with mucormycosis infections in horses, cattle, sheep, goats, dogs and cats including diseases or conditions such as mucormycotic ruminitis, lymphadentitis, and enteritis, wherein said method comprises selecting a subject in need of treating or preventing mucormycosis infections and administering to the subject a composition comprising an effective amount of one or more compounds according to the present invention and an excipient, thereby treating or preventing mucormycosis infections in the subject.

The present invention also relates to a method for treating or preventing coccidioidomycosis in dogs and cats caused by infection with an organism selected from the group consisting of Coccidioides immitis and Coccidioides posadasii, said method comprising selecting a subject in need of treating or preventing coccidioidomycosis and administering to the subject an effective amount of a compound or composition according to the present invention, thereby treating or preventing coccidioidomycosis in the subject.

The present invention also relates to a method for treating or preventing coccidioidomycosis in dogs and cats caused by infection with an organism selected from the group consisting of Coccidioides immitis and Coccidioides posadasii, wherein said method comprises selecting a subject in need of treating or preventing coccidioidomycosis and administering to the subject a composition comprising an effective amount of one or more compounds according to the present invention and an excipient, thereby treating or preventing coccidioidomycosis in the subject.

The present invention also relates to a method for treating or preventing blastomycosis in dogs and cats caused by infection with Blastomyces dermatitidis, said method comprising selecting a subject in need of treating or preventing blastomycosis and administering to the subject an effective amount of a compound or composition according to the present invention, thereby treating or preventing blastomycosis in the subject.

The present invention also relates to a method for treating or preventing blastomycosis in dogs and cats caused by infection with Blastomyces dermatitidis, wherein said method comprises selecting a subject in need of treating or preventing blastomycosis and administering to a subject a composition comprising an effective amount of one or more compounds according to the present invention and an excipient, thereby treating or preventing blastomycosis in the subject.

The present invention also relates to a method for treating or preventing Paracoccidioidomycosis in dogs caused by infection with Paracoccidioides brasiliensis, said method comprising selecting a subject in need of treating or preventing Paracoccidioidomycosis and administering to a subject an effective amount of a compound or composition according to the present invention, thereby treating or preventing Paracoccidioidomycosis in the subject.

The present invention also relates to a method for treating or preventing Paracoccidioidomycosis in dogs caused by infection with Paracoccidioides brasiliensis, wherein said method comprises selecting a subject in need of treating or preventing Paracoccidioidomycosis and administering to a subject a composition comprising an effective amount of one or more compounds according to the present invention and an excipient, thereby treating or preventing Paracoccidioidomycosis in the subject.

The present invention also relates to a method for treating or preventing dermatophytosis (ringworm) in cats and dogs caused by infection with an organism selected from the group consisting of Microsporum canis, Microsporum gypseum, and Trichophyton mentagrophytes, said method comprising selecting a subject in need of treating or preventing dermatophytosis (ringworm) and administering to a subject an effective amount of a compound or composition according to the present invention, thereby treating or preventing dermatophytosis (ringworm) in the subject.

The present invention also relates to a method for treating or preventing dermatophytosis (ringworm) in cats and dogs caused by infection with an organism selected from the group consisting of Microsporum canis, Microsporum gypseum, and Trichophyton mentagrophytes, wherein said method comprises selecting a subject in need of treating or preventing dermatophytosis (ringworm) and administering to a subject a composition comprising an effective amount of one or more compounds according to the present invention and an excipient, thereby treating or preventing dermatophytosis (ringworm) in the subject.

The present invention also relates to a method for treating or preventing cryptococcosis in dogs and cats caused by infection with an organism selected from the group consisting of Cryptococcus neoformans and Cryptococcus gattii, said method comprising selecting a subject in need of treating or preventing cryptococcosis and administering to a subject an effective amount of a compound or composition according to the present invention, thereby treating or preventing cryptococcosis in the subject.

The present invention also relates to a method for treating or preventing cryptococcosis in dogs and cats caused by infection with an organism selected from the group consisting of Cryptococcus neoformans and Cryptococcus gattii, wherein said method comprises selecting a subject in need of treating or preventing cryptococcosis and administering to a subject a composition comprising an effective amount of one or more compounds according to the present invention and an excipient, thereby treating or preventing cryptococcosis in the subject.

The present invention also relates to a method for treating or preventing histoplasmosis in dogs caused by infection with Histoplasma capsulatum, said method comprising selecting a subject in need of treating or preventing histoplasmosis and administering to a subject an effective amount of a compound or composition according to the present invention, thereby treating or preventing histoplasmosis in the subject.

The present invention also relates to a method for treating or preventing histoplasmosis in dogs caused by infection with Histoplasma capsulatum, wherein said method comprises selecting a subject in need of treating or preventing histoplasmosis and administering to a subject a composition comprising an effective amount of one or more compounds according to the present invention and an excipient, thereby treating or preventing histoplasmosis in the subject.

The present invention further relates to a process for preparing the antifungal agents of the present invention.

The disclosure provides for the use of the compositions of the disclosure for the production of a medicament for preventing and/or treating the indications as set forth herein.

In accordance with a further embodiment, the present disclosure provides a use of the pharmaceutical compositions described herein, in an amount effective for use in a medicament, and most preferably for use as a medicament for treating a disease or disorder, for example, as set forth in herein, in a subject.

In accordance with yet another embodiment, the present disclosure provides a use of the pharmaceutical compositions described above, and at least one additional therapeutic agent, in an amount effective for use in a medicament, and most preferably for use as a medicament for treating a disease or disorder associated with disease, for example, as set forth herein, in a subject.

The disclosure provides a method for treating and/or preventing a disease or condition as set forth herein in a subject, wherein said method comprises selecting a subject in need of treating and/or preventing said disease or condition as set forth herein; administering to the subject a composition of the disclosure in a therapeutically effective amount, thereby treating and/or preventing said disease in said subject.

These and other objects, features, and advantages will become apparent to those of ordinary skill in the art from a reading of the following detailed description and the appended claims. All percentages, ratios and proportions herein are by weight, unless otherwise specified. All temperatures are in degrees Celsius (° C.) unless otherwise specified. All documents cited are in relevant part, incorporated herein by reference; the citation of any document is not to be construed as an admission that it is prior art with respect to the present invention.

### DETAILED DESCRIPTION OF THE INVENTION

The compounds of the disclosure act on pathogenic fungi to suppress their growth. The compounds of the disclosure can also kill fungi. As antifungal agents, the compounds of the disclosure can be used to treat local, topical and disseminated infections in animals including humans and can be used to prevent disseminated fungal infections developing from local or topical fungal infections. In another aspect of this invention, the compounds can be applied to agricultural plants, shrubs and trees to cure and prevent fungal infections and fungal diseases.

Throughout the description, where compositions are described as having, including, or comprising specific components, or where processes are described as having, including, or comprising specific process steps, it is contemplated that compositions of the present teachings also consist essentially of, or consist of, the recited components, and that the processes of the present teachings also consist essentially of, or consist of, the recited processing steps.

In the application, where an element or component is said to be included in and/or selected from a list of recited elements or components, it should be understood that the element or component can be any one of the recited elements or components and can be selected from a group consisting of two or more of the recited elements or components.

The use of the singular herein includes the plural (and vice versa) unless specifically stated otherwise. In addition, where the use of the term “about” is before a quantitative value, the present teachings also include the specific quantitative value itself, unless specifically stated otherwise.

It should be understood that the order of steps or order for performing certain actions is immaterial so long as the present teachings remain operable. Moreover, two or more steps or actions can be conducted simultaneously

As used herein, the term “halogen” shall mean chlorine, bromine, fluorine and iodine.

As used herein, unless otherwise noted, “alkyl” and/or “aliphatic” whether used alone or as part of a substituent group refers to straight and branched carbon chains having 1 to 20 carbon atoms or any number within this range, for example 1 to 6 carbon atoms or 1 to 4 carbon atoms. Designated numbers of carbon atoms (e.g. C₁₋₆) shall refer independently to the number of carbon atoms in an alkyl moiety or to the alkyl portion of a larger alkyl-containing substituent. Non-limiting examples of alkyl groups include methyl, ethyl, n-propyl, iso-propyl, n-butyl, sec-butyl, iso-butyl, tert-butyl, and the like. Alkyl groups can be optionally substituted. Non-limiting examples of substituted alkyl groups include hydroxymethyl, chloromethyl, trifluoromethyl, aminomethyl, 1-chloroethyl, 2-hydroxyethyl, 1,2-difluoroethyl, 3-carboxypropyl, and the like. In substituent groups with multiple alkyl groups such as (C₁₋₆alkyl)₂amino, the alkyl groups may be the same or different.

As used herein, the terms “alkenyl” and “alkynyl” groups, whether used alone or as part of a substituent group, refer to straight and branched carbon chains having 2 or more carbon atoms, preferably 2 to 20, wherein an alkenyl chain has at least one double bond in the chain and an alkynyl chain has at least one triple bond in the chain. Alkenyl and alkynyl groups can be optionally substituted. Nonlimiting examples of alkenyl groups include ethynyl, 3-propenyl, 1-propenyl (also 2-methylethenyl), isopropenyl (also 2-methylethen-2-yl), buten-4-yl, and the like. Nonlimiting examples of substituted alkenyl groups include 2-chloroethenyl (also 2-chlorovinyl), 4-hydroxybuten-1-yl, 7-hydroxy-7-methyloct-4-en-2-yl, 7-hydroxy-7-methyloct-3,5-dien-2-yl, and the like. Nonlimiting examples of alkynyl groups include ethynyl, prop-2-ynyl (also propargyl), propyn-1-yl, and 2-methyl-hex-4-yn-1-yl. Nonlimiting examples of substituted alkynyl groups include, 5-hydroxy-5-methylhex-3-ynyl, 6-hydroxy-6-methylhept-3-yn-2-yl, 5-hydroxy-5-ethylhept-3-ynyl, and the like.

As used herein, “cycloalkyl,” whether used alone or as part of another group, refers to a non-aromatic carbon-containing ring including cyclized alkyl, alkynyl, and alkynyl groups, e.g., having from 3 to 14 ring carbon atoms, preferably from 3 to 7 or 3 to 6 ring carbon atoms, or even 3 to 4 ring carbon atoms, and optionally containing one or more (e.g., 1, 2, or 3) double or triple bond. Cycloalkyl groups can be monocyclic (e.g., cyclohexyl) or polycyclic (e.g., containing fused, bridged, and/or Spiro ring systems), wherein the carbon atoms are located inside or outside of the ring system. Any suitable ring position of the cycloalkyl group can be covalently linked to the defined chemical structure. Cycloalkyl rings can be optionally substituted. Nonlimiting examples of cycloalkyl groups include: cyclopropyl, 2-methyl-cyclopropyl, cyclopropenyl, cyclobutyl, 2,3-dihydroxycyclobutyl, cyclobutenyl, cyclopentyl, cyclopentenyl, cyclopentadienyl, cyclohexyl, cyclohexenyl, cycloheptyl, cyclooctanyl, decalinyl, 2,5-dimethylcyclopentyl, 3,5-dichlorocyclohexyl, 4-hydroxycyclohexyl, 3,3,5-trimethylcyclohex-1-yl, octahydropentalenyl, octahydro-1H-indenyl, 3a,4,5,6,7,7a-hexahydro-3H-inden-4-yl, decahydroazulenyl; bicyclo[6.2.0]decanyl, decahydronaphthalenyl, and dodecahydro-1H-fluorenyl. The term “cycloalkyl” also includes carbocyclic rings which are bicyclic hydrocarbon rings, non-limiting examples of which include, bicyclo-[2.1.1]hexanyl, bicyclo[2.2.1]heptanyl, bicyclo[3.1.1]heptanyl, 1,3-dimethyl[2.2.1]heptan-2-yl, bicyclo[2.2.2]octanyl, and bicyclo[3.3.3]undecanyl.

“Haloalkyl” is intended to include both branched and straight-chain saturated aliphatic hydrocarbon groups having the specified number of carbon atoms, substituted with 1 or more halogen. Haloalkyl groups include perhaloalkyl groups, wherein all hydrogens of an alkyl group have been replaced with halogens (e.g., —CF₃, —CF₂CF₃). Haloalkyl groups can optionally be substituted with one or more substituents in addition to halogen. Examples of haloalkyl groups include, but are not limited to, fluoromethyl, dichloroethyl, trifluoromethyl, trichloromethyl, pentafluoroethyl, and pentachloroethyl groups.

The term. “alkoxy” refers to the group —O-alkyl, wherein the alkyl group is as defined above. Alkoxy groups optionally may be substituted. The term C₃-C₆ cyclic alkoxy refers to a ring containing 3 to 6 carbon atoms and at least one oxygen atom (e.g., tetrahydrofuran, tetrahydro-2H-pyran). C₃-C₆ cyclic alkoxy groups optionally may be substituted.

The term “aryl,” wherein used alone or as part of another group, is defined herein as a an unsaturated, aromatic monocyclic ring of 6 carbon members or to an unsaturated, aromatic polycyclic ring of from 10 to 14 carbon members. Aryl rings can be, for example, phenyl or naphthyl ring each optionally substituted with one or more moieties capable of replacing one or more hydrogen atoms. Non-limiting examples of aryl groups include: phenyl, naphthylen-1-yl, naphthylen-2-yl, 4-fluorophenyl, 2-hydroxyphenyl, 3-methylphenyl, 2-amino-4-fluorophenyl, 2-(N,N-diethylamino)phenyl, 2-cyanophenyl, 2,6-di-tert-butylphenyl, 3-methoxyphenyl, 8-hydroxynaphthylen-2-yl 4,5-dimethoxynaphthylen-1-yl, and 6-cyano-naphthylen-1-yl. Aryl groups also include, for example, phenyl or naphthyl rings fused with one or more saturated or partially saturated carbon rings (e.g., bicyclo[4.2.0]octa-1,3,5-trienyl, indanyl), which can be substituted at one or more carbon atoms of the aromatic and/or saturated or partially saturated rings.

The term “arylalkyl” or “aralkyl” refers to the group -alkyl-aryl, where the alkyl and aryl groups are as defined herein. Aralkyl groups of the present invention are optionally substituted. Examples of arylalkyl groups include, for example, benzyl, 1-phenylethyl, 2-phenylethyl, 3-phenylpropyl, 2-phenylpropyl, fluorenylmethyl and the like.

The terms “heterocyclic” and/or “heterocycle” and/or “heterocyclyl,” whether used alone or as part of another group, are defined herein as one or more ring having from 3 to 20 atoms wherein at least one atom in at least one ring is a heteroatom selected from nitrogen (N), oxygen (O), or sulfur (S), and wherein further the ring that includes the heteroatom is non-aromatic. In heterocycle groups that include 2 or more fused rings, the non-heteroatom bearing ring may be aryl (e.g., indolinyl, tetrahydroquinolinyl, chromanyl). Exemplary heterocycle groups have from 3 to 14 ring atoms of which from 1 to 5 are heteroatoms independently selected from nitrogen (N), oxygen (O), or sulfur (S). One or more N or S atoms in a heterocycle group can be oxidized. Heterocycle groups can be optionally substituted.

Non-limiting examples of heterocyclic units having a single ring include: diazirinyl, aziridinyl, urazolyl, azetidinyl, pyrazolidinyl, imidazolidinyl, oxazolidinyl, isoxazolinyl, isoxazolyl, thiazolidinyl, isothiazolyl, isothiazolinyl oxathiazolidinonyl, oxazolidinonyl, hydantoinyl, tetrahydropyranyl, pyrrolidinyl, morpholinyl, piperazinyl, piperidinyl, dihydropyranyl, tetrahydropyranyl, piperidin-2-onyl (valerolactam), 2,3,4,5-tetrahydro-1H-azepinyl, 2,3-dihydro-1H-indole, and 1,2,3,4-tetrahydro-quinoline. Non-limiting examples of heterocyclic units having 2 or more rings include: hexahydro-1H-pyrrolizinyl, 3a,4,5,6,7,7a-hexahydro-1H-benzo[d]imidazolyl, 3a,4,5,6,7,7a-hexahydro-1H-indolyl, 1,2,3,4-tetrahydroquinolinyl, chromanyl, isochromanyl, indolinyl, isoindolinyl, and decahydro-1H-cycloocta[b]pyrrolyl.

The term “heteroaryl.” whether used alone or as part of another group, is defined herein as one or more rings having from 5 to 20 atoms wherein at least one atom in at least one ring is a heteroatom chosen from nitrogen (N), oxygen (O), or sulfur (S), and wherein further at least one of the rings that includes a heteroatom is aromatic. In heteroaryl groups that include 2 or more fused rings, the non-heteroatom bearing ring may be a carbocycle (e.g., 6,7-Dihydro-5H-cyclopentapyrimidine) or anal (e.g., benzofuranyl, benzothiophenyl, indolyl). Exemplary heteroaryl groups have from 5 to 14 ring atoms and contain from 1 to 5 ring heteroatoms independently selected from nitrogen (N), oxygen (O), or sulfur (S). One or more N or S atoms in a heteroaryl group can be oxidized. Heteroaryl groups can be substituted. Non-limiting examples of heteroaryl rings containing a single ring include: 1,2,3,4-tetrazolyl, [1,2,3]triazolyl, [1,2,4]triazolyl, triazinyl, thiazolyl, 1H-imidazolyl, oxazolyl, furanyl, thiophenyl, pyrimidinyl, 2-phenylpyrimidinyl, pyridinyl, 3-methylpyridinyl, and 4-dimethylaminopyridinyl. Non-limiting examples of heteroaryl rings containing 2 or more fused rings include: benzofuranyl, benzothiophenyl, benzoxazolyl, benzthiazolyl, benztriazolyl, cinnolinyl, naphthyridinyl, phenanthridinyl, 7H-purinyl, 9H-purinyl, 6-amino-9H-purinyl, 5H-pyrrolo[3,2-d]pyrimidinyl, 7H-pyrrolo[2,3-d]pyrimidinyl, pyrido[2,3-d]pyrimidinyl, 2-phenylbenzo[d]thiazolyl, 1H-indolyl, 4,5,6,7-tetrahydro-1-H-indolyl, quinoxalinyl, 5-methylquinoxalinyl, quinazolinyl, quinolinyl, 8-hydroxy-quinolinyl, and isoquinolinyl.

One non-limiting example of a heteroaryl group as described above is C₁-C₅ heteroaryl, which has 1 to 5 carbon ring atoms and at least one additional ring atom that is a heteroatom (preferably 1 to 4 additional ring atoms that are heteroatoms) independently selected from nitrogen (N), oxygen (O), or sulfur (S). Examples of C₁-C₅ heteroaryl include, but are not limited to, triazinyl, thiazol-2-yl, thiazol-4-yl, imidazol-1-yl, 1H-imidazol-2-yl, 1H-imidazol-4-yl, isoxazolin-5-yl, furan-2-yl, furan-3-yl, thiophen-2-yl, thiophen-4-yl, pyrimidin-2-yl, pyrimidin-4-yl, pyrimidin-5-yl, pyridin-2-yl, pyridin-3-yl, and pyridin-4-yl.

Unless otherwise noted, when two substituents are taken together to form a ring having a specified number of ring atoms (e.g., R² and R³ taken together with the nitrogen (N) to which they are attached to form a ring having from 3 to 7 ring members), the ring can have carbon atoms and optionally one or more (e.g., 1 to 3) additional heteroatoms independently selected from nitrogen (N), oxygen (O), or sulfur (S). The ring can be saturated or partially saturated and can be optionally substituted.

For the purposed of the present invention fused ring units, as well as spirocyclic rings, bicyclic rings and the like, which comprise a single heteroatom will be considered to belong to the cyclic family corresponding to the heteroatom containing ring. For example, 1,2,3,4-tetrahydroquinoline having the formula:

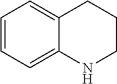

is, for the purposes of the present invention, considered a heterocyclic unit. 6,7-Dihydro-5H-cyclopentapyrimidine having the formula:

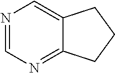

is, for the purposes of the present invention, considered a heteroaryl unit. When a fused ring unit contains heteroatoms in both a saturated and an aryl ring, the aryl ring will predominate and determine the type of category to which the ring is assigned. For example, 1,2,3,4-tetrahydro-[1,8]naphthridine having the formula:

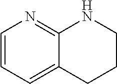

is, for the purposes of the present invention, considered a heteroaryl unit.

Whenever a term or either of their prefix roots appear in a name of a substituent the name is to be interpreted as including those limitations provided herein. For example, whenever the term “alkyl” or “aryl” or either of their prefix roots appear in a name of a substituent (e.g., arylalkyl, alkylamino) the name is to be interpreted as including those limitations given above for “alkyl” and “aryl.”

The term “substituted” is used throughout the specification. The term “substituted” is defined herein as a moiety, whether acyclic or cyclic, which has one or more hydrogen atoms replaced by a substituent or several (e.g., 1 to 10) substituents as defined herein below. The substituents are capable of replacing one or two hydrogen atoms of a single moiety at a time. In addition, these substituents can replace two hydrogen atoms on two adjacent carbons to form said substituent, new moiety or unit. For example, a substituted unit that requires a single hydrogen atom replacement includes halogen, hydroxyl, and the like. A two hydrogen atom replacement includes carbonyl, oximino, and the like. A two hydrogen atom replacement from adjacent carbon atoms includes epoxy, and the like. The term “substituted” is used throughout the present specification to indicate that a moiety can have one or more of the hydrogen atoms replaced by a substituent. When a moiety is described as “substituted” any number of the hydrogen atoms may be replaced. For example, difluoromethyl is a substituted C₁ alkyl; trifluoromethyl is a substituted C₁ alkyl; 4-hydroxyphenyl is a substituted aromatic ring; (N,N-dimethyl-5-amino)octanyl is a substituted C₈ alkyl; 3-guanidinopropyl is a substituted C₃ alkyl; and 2-carboxypyridinyl is a substituted heteroaryl.

The variable groups defined herein, e.g., alkyl, alkenyl, alkynyl, cycloalkyl, alkoxy, aryloxy, aryl, heterocycle and heteroaryl groups defined herein, whether used alone or as part of another group, can be optionally substituted. Optionally substituted groups will be so indicated.

The following are non-limiting examples of substituents which can substitute for hydrogen atoms on a moiety:halogen (chlorine (Cl), bromine (Br), fluorine (F) and iodine(I)), —CN, —NO₂, oxo (═O), OR¹⁴, SR¹⁴, —N(R¹⁴)₂, —NR¹⁴C(O)R¹⁴, SO₂R¹⁴, —SO₂OR¹⁴, —SO₂N(R¹⁴)₂, —C(O)R¹⁴, —C(O)OR¹⁴, —C(O)N(R¹⁴)₂, C₁₋₆ alkyl, C₁₋₆ haloalkyl, C₁₋₆ alkoxy, C₂₋₈alkenyl, C₂₋₈alkynyl, C₃₋₁₄ cycloalkyl, aryl, heterocycle, or heteroaryl, wherein each of the alkyl, haloalkyl, alkenyl, alkynyl, alkoxy, cycloalkyl, aryl, heterocycle, and heteroaryl groups is optionally substituted with 1-10 (e.g., 1-6 or 1-4) groups selected independently from halogen, —CN, —NO₂, oxo, and R¹⁴; wherein R¹⁴, at each occurrence, independently is hydrogen, —OR¹⁵, —SR¹⁵, —C(O)R¹³, —C(O)OR¹⁵, —C(O)N(R¹⁵)₂, —SO₂R¹⁵, —S(O)₂OR¹⁵, —N(R¹⁵)₂, —NR¹⁵C(O)R¹⁵, C₁₋₆ alkyl, C₁₋₆ haloalkyl, C₂₋₈ alkenyl, C₂₋₈alkenyl, cycloalkyl (e.g., C₃₋₆ cycloalkyl), aryl, heterocycle, or heteroaryl, or two R¹⁴ units taken together with the atom(s) to which they are bound form an optionally substituted carbocycle or heterocycle wherein said carbocycle or heterocycle has 3 to 7 ring atoms; wherein R¹⁵, at each occurrence, independently is hydrogen, C₁₋₆ alkyl, C₁₋₆ haloalkyl, C₂₋₈ alkenyl, C₂₋₈alkenyl, cycloalkyl (e.g., C₃₋₆ cycloalkyl), aryl, heterocycle, or heteroaryl, or two R¹⁵ units taken together with the atom(s) to which they are bound form an optionally substituted carbocycle or heterocycle wherein said carbocycle or heterocycle preferably has 3 to 7 ring atoms.

In some embodiments, the substituents are selected from
i) —OR¹⁶; for example, —OH, —OCH₃, —OCH₂CH₃, —OCH₂CH₂CH₃;
ii) —C(O)R¹⁶; for example, —COCH₃, —COCH₂CH₃, —COCH₂CH₂CH₃;
iii) —C(O)OR¹⁶; for example, —CO₂CH₃, —CO₂CH₂CH₃, —CO₂CH₂CH₂CH₃;
iv) —C(O)N(R¹⁶)₂; for example, —CONH₂, —CONHCH₃, —CON(CH₃)₂;
v) —N(R¹⁶)₂; for example, —NH₂, —NHCH₃, —N(CH₃)₂, —NH(CH₂CH₃);
vi) halogen: —F, —Cl, —Br, and —I;
vii) —CHₑX_{g}; wherein X is halogen, m is from 0 to 2, e+g=3; for example, —CH₂F, —CHF₂, —CF₃, —CCl₃, or —CBr₃;
viii) —SO₂R¹⁶; for example, —SO₂H; —SO₂CH₃; —SO₂C₆H₅;
ix) C₁-C₆ linear, branched, or cyclic alkyl;
x) Cyano
xi) Nitro;
xii) N(R¹⁶)C(O)R¹⁶;
xiii) Oxo (═O);
xiv) Heterocycle;
xv) Heteroaryl; and
xvi) SR¹⁰.
wherein each R¹⁶ is independently hydrogen, optionally substituted C₁-C₆ linear or branched alkyl (e.g., optionally substituted C₁-C₄ linear or branched alkyl), or optionally substituted C₃-C₆ cycloalkyl (e.g optionally substituted C₃-C₄ cycloalkyl); or two R¹⁶ units can be taken together to form a ring comprising 3-7 ring atoms. In certain aspects, each R¹⁶ is independently hydrogen, C₁-C₆ linear or branched alkyl optionally substituted with halogen or C₃-C₆ cycloalkyl or C₃-C₆ cycloalkyl.

At various places in the present specification, substituents of compounds are disclosed in groups or in ranges. It is specifically intended that the description include each and every individual subcombination of the members of such groups and ranges. For example, the term “C₁₋₆ alkyl” is specifically intended to individually disclose C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₅, C₃-C₄, C₄-C₆, C₄-C₅, and C₅-C₆, alkyl.

For the purposes of the present invention the terms “compound,” “analog,” and “composition of matter” stand equally well for the antifungal agent described herein, including all enantiomeric forms, diastereomeric forms, salts, and the like, and the terms “compound,” “analog,” and “composition of matter” are used interchangeably throughout the present specification.

Compounds described herein can contain an asymmetric atom (also referred as a chiral center), and some of the compounds can contain one or more asymmetric atoms or centers, which can thus give rise to optical isomers (enantiomers) and diastereomers. The present teachings and compounds disclosed herein include such enantiomers and diastereomers, as well as the racemic and resolved, enantiomerically pure R and S stereoisomers, as well as other mixtures of the R and S stereoisomers and pharmaceutically acceptable salts thereof. Optical isomers can be obtained in pure form by standard procedures known to those skilled in the art, which include, but are not limited to, diastereomeric salt formation, kinetic resolution, and asymmetric synthesis. The present teachings also encompass cis and trans isomers of compounds containing alkenyl moieties (e.g., alkenes and imines). It is also understood that the present teachings encompass all possible regioisomers, and mixtures thereof, which can be obtained in pure form by standard separation procedures known to those skilled in the art, and include, but are not limited to, column chromatography, thin-layer chromatography, and high-performance liquid chromatography.

Pharmaceutically acceptable salts of compounds of the present teachings, which can have an acidic moiety, can be formed using organic and inorganic bases. Both mono and polyanionic salts are contemplated, depending on the number of acidic hydrogens available for deprotonation. Suitable salts formed with bases include metal salts, such as alkali metal or alkaline earth metal salts, for example sodium, potassium, or magnesium salts; ammonia salts and organic amine salts, such as those formed with morpholine, thiomorpholine, piperidine, pyrrolidine, a mono-, di- or tri-lower alkylamine (e.g., ethyl-tert-butyl-, diethyl-, diisopropyl-, triethyl-, tributyl- or dimethylpropylamine), or a mono-, di-, or trihydroxy lower alkylamine (e.g., mono-, di- or triethanolamine). Specific non-limiting examples of inorganic bases include NaHCO₃, Na₂CO₃, KHCO₃, K₂CO₃, Cs₂CO₃, LiOH, NaOH, KOH, NaH₂PO₄, Na₂HPO₄, and Na₃PO₄. Internal salts also can be formed. Similarly, when a compound disclosed herein contains a basic moiety, salts can be formed using organic and inorganic acids. For example, salts can be formed from the following acids: acetic, propionic, lactic, benzenesulfonic, benzoic, camphorsulfonic, citric, tartaric, succinic, dichloroacetic, ethenesulfonic, formic, fumaric, gluconic, glutamic, hippuric, hydrobromic, hydrochloric, isethionic, lactic, maleic, malic, malonic, mandelic, methanesulfonic, mucic, naphthalenesulfonic, nitric, oxalic, pamoic, pantothenic, phosphoric, phthalic, propionic, succinic, sulfuric, tartaric, toluenesulfonic, and camphorsulfonic as well as other known pharmaceutically acceptable acids.

When any variable occurs more than one time in any constituent or in any formula, its definition in each occurrence is independent of its definition at every other occurrence (e.g., in N(R⁹)₂, each R⁹ may be the same or different than the other). Combinations of substituents and/or variables are permissible only if such combinations result in stable compounds.

The terms “treat” and “treating” and “treatment” as used herein, refer to partially or completely alleviating, inhibiting, ameliorating and/or relieving a condition from which a patient is suspected to suffer.

As used herein, “therapeutically effective” and “effective dose” refer to a substance or an amount that elicits a desirable biological activity or effect.

Except when noted, the terms “subject” or “patient” are used interchangeably and refer to mammals such as human patients and non-human primates, as well as experimental animals such as rabbits, rats, and mice, and other animals. Accordingly, the term. “subject” or “patient” as used herein means any mammalian patient or subject to which the compounds of the invention can be administered. In an exemplary embodiment of the present invention, to identify subject patients for treatment according to the methods of the invention, accepted screening methods are employed to determine risk factors associated with a targeted or suspected disease or condition or to determine the status of an existing disease or condition in a subject. These screening methods include, for example, conventional work-ups to determine risk factors that may be associated with the targeted or suspected disease or condition. These and other routine methods allow the clinician to select patients in need of therapy using the methods and compounds of the present invention.

### Antifungal Agents

The antifungal agents of the present invention are heteroaromatic compounds, and include all enantiomeric and diastereomeric forms and pharmaceutically accepted salts thereof having the formula (I):

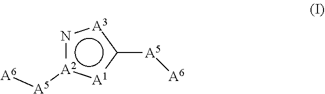

Including hydrates, solvates, pharmaceutically acceptable salts, prodrugs and complexes thereof, wherein:
A¹ is selected from the group consisting of CR¹, O, N, and NR¹;
When A¹ is CR¹, A², A³ are N;
Alternately, when A¹ is CR¹, AZ is C, and A³ is NR^{1a};
When A¹ is O, A³ is C, and A³ is N;
When A¹ is NR¹, AZ is C, and A³ is N;
When A¹ is N, A³ is C, and A³ is NR^{1a};
A⁵ is at each occurrence independently selected from the group consisting of

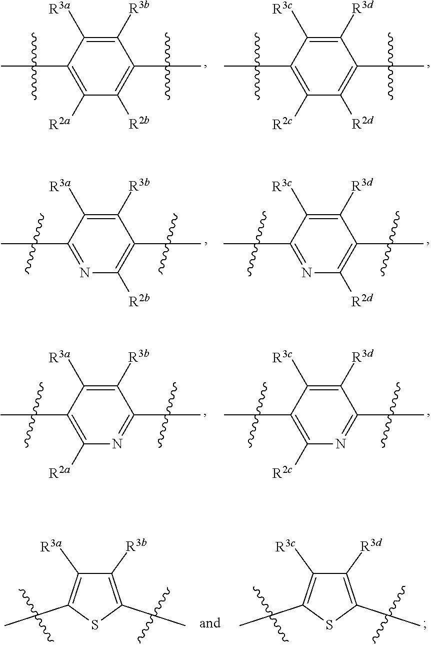

A⁶ is at each occurrence independently selected from the group consisting of,

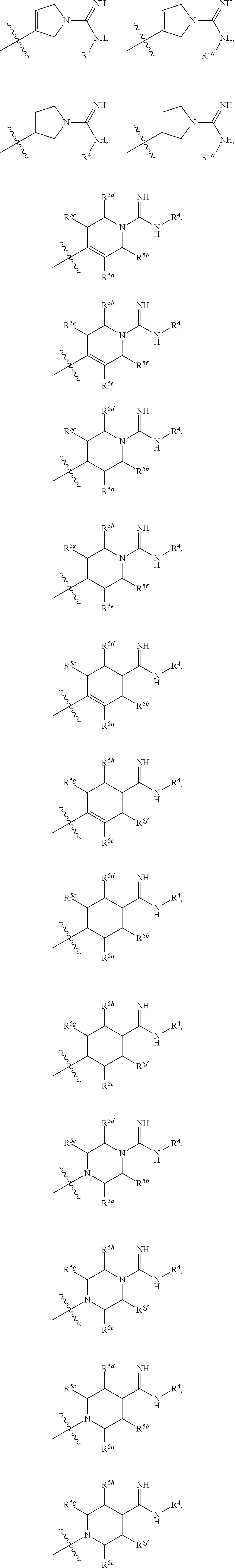

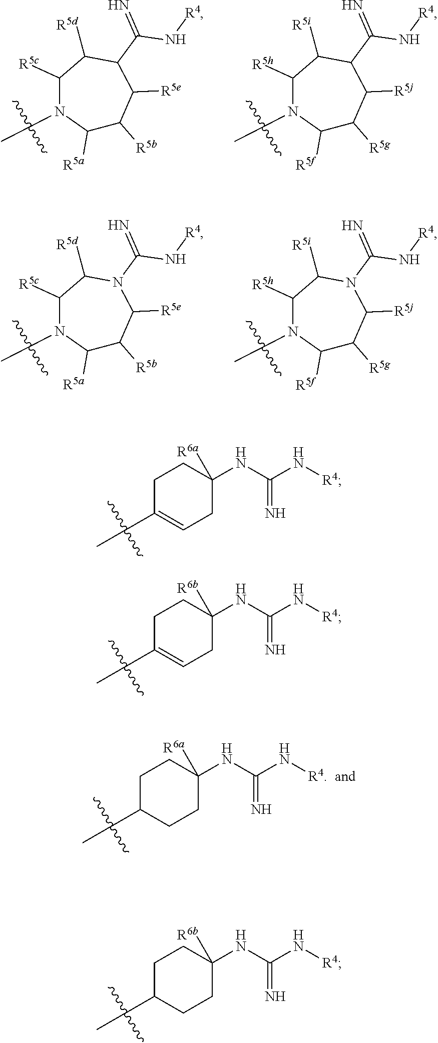

R¹ is selected from the group consisting of hydrogen C₁₋₈ alkyl, C₃₋₈ branched alkyl, C₃₋₈ cycloalkyl, optionally substituted benzyl,

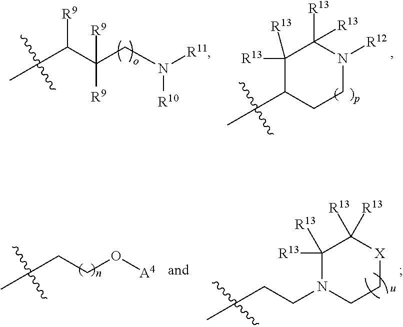

R^{1a} is selected from the group consisting of hydrogen, C₁₋₈ alkyl. C₃₋₈ branched alkyl, C₃₋₈ cycloalkyl, optionally substituted benzyl,

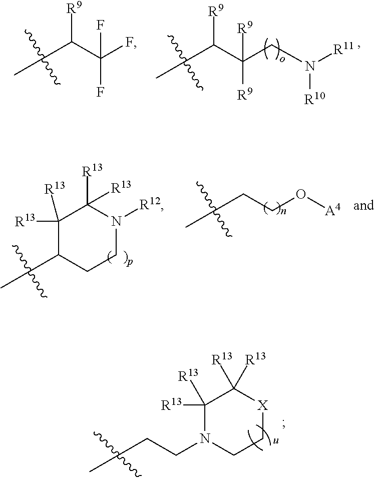

A⁴ is selected from the group consisting of hydrogen, C₁₋₄ alkyl, C₃₋₅ branched alkyl, C₃₋₈ cycloalkyl,

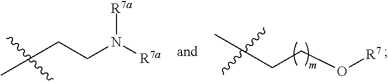

R⁷ is selected from the group consisting of hydrogen C₁₋₄ alkyl and C₃₋₅ branched alkyl;
R^{7a} is selected from the group consisting of C₁₋₄ alkyl, C₃₋₈ branched alkyl, and C₃₋₈ cycloalkyl;
R⁸ is at each occurrence independently selected from the group consisting of hydrogen C₁₋₄ alkyl;
R^{8a} is selected from the group consisting of hydrogen, C₁₋₄ alkyl, and

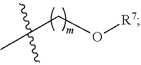

R⁹ is at each occurrence independently selected from the group consisting of hydrogen, and C₁₋₄ alkyl;
R¹⁰ is selected from the group consisting of hydrogen, C₁₋₄ alkyl, C₃₋₈ branched alkyl, and C₃₋₈ cycloalkyl;
R¹¹ is selected from the group consisting of C₁₋₄ alkyl, C₃₋₈ branched alkyl, C₃₋₈ cycloalkyl, and

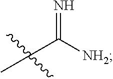 [SMILES: *C(=N)N]

In some embodiments R¹⁰ and R¹¹ are optionally joined to form a heterocyclic ring consisting of three, four, five, six, or seven members;
R¹² is selected from the group consisting of C₁₋₄ alkyl, C₃₋₈ branched alkyl, C₃₋₈ cycloalkyl, and,

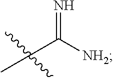 [SMILES: *C(=N)N]

R¹³ is at each occurrence independently selected from the group consisting of hydrogen and C₁₋₄ alkyl;
p is 0, 1, or 2;
o is 0, 1, or 2;
n is 0; 1, or 2;
m is 1, 2, or 3;
u is 1 or 2;
X is selected from the group consisting of NR¹², oxygen, sulfur, and SO₂;
R^{2a} is selected from the group consisting of hydrogen, C₁₋₄ alkyl, fluorine, chlorine, C₁₋₄-alkoxy, CN, OCF₃ and CF₃;
R^{2b} is selected from the group consisting of hydrogen, C₁₋₄ alkyl, fluorine, chlorine, C₁₋₄-alkoxy, CN, OCF₃ and CF₃;
R^{2c} is selected from the group consisting of hydrogen, C₁₋₄ alkyl, fluorine, chlorine, C₁₋₄-alkoxy, CN, OCF₃ and CF₃;
R^{2d} is selected from the group consisting of hydrogen, C₁₋₄ alkyl, fluorine, chlorine, C₁₋₄-alkoxy, CN, OCF₃ and CF₃;
R^{3a} is selected from the group consisting of hydrogen, C₁₋₄alkyl, fluorine, chlorine, C₁₋₄-alkoxy, CN, OCF₃ and CF₃;
R^{3b} is selected from the group consisting of hydrogen, C₁₋₄ alkyl, fluorine, chlorine. C₁₋₄-alkoxy, CN, OCF₃ and CF₃;
R^{3c} is selected from the group consisting of hydrogen, C₁₋₄ alkyl, fluorine, chlorine, C₁₋₄-alkoxy, CN, OCF₃ and CF₃;
R^{3d} is selected from the group consisting of hydrogen, C₁₋₄ alkyl, fluorine, chlorine, C₁₋₄-alkoxy, CN, OCF₃ and CF₃;
R⁴ is selected from the group consisting of hydrogen and C₁₋₄ alkyl;
R^{4a} is selected from the group consisting of hydrogen and C₁₋₄ alkyl;
R^{5a} is selected from the group consisting of hydrogen and C₁₋₄ alkyl;
R^{5b} is selected from the group consisting of hydrogen and C₁₋₄ alkyl;
R^{5c} is selected from the group consisting of hydrogen and C₁₋₄ alkyl;
R^{5d} is selected from the group consisting of hydrogen and C₁₋₄ alkyl;
R^{5e} is selected from the group consisting of hydrogen and C₁₋₄ alkyl;
R^{5f} is selected from the group consisting of hydrogen and C₁₋₄alkyl;
R^{5g} is selected from the group consisting of hydrogen and C₁₋₄ alkyl;
R^{5h} is selected from the group consisting of hydrogen and C₁₋₄ alkyl;
R^{6a} is selected from the group consisting of hydrogen and C₁₋₄ alkyl;
R^{6b} is selected from the group consisting of hydrogen and C₁₋₄ alkyl.

The compounds of the present invention include compounds having formula (II):

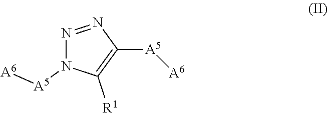

including enantiomers, diastereomers, hydrates, solvates, pharmaceutically acceptable salts, and complexes thereof.

The compounds of the present invention include compounds having formula (III):

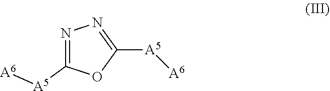

including enantiomers, diastereomers, hydrates, solvates, pharmaceutically acceptable salts, and complexes thereof.

The compounds of the present invention include compounds having formula (IV):

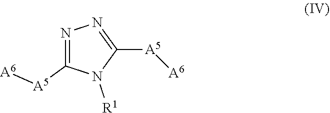

including enantiomers, diastereomers, hydrates, solvates pharmaceutically acceptable salts, and complexes thereof.

The compounds of the present invention include compounds having formula (V):

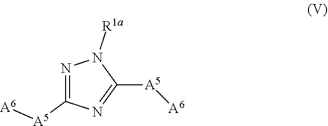

including enantiomers, diastereomers, hydrates, solvates, pharmaceutically acceptable salts, and complexes thereof.

The compounds of the present invention include compounds having formula (VI):

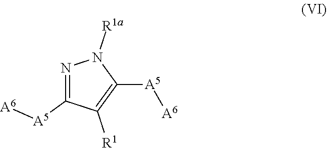

including enantiomers, diastereomers, hydrates, solvates, pharmaceutically acceptable salts, and complexes thereof.

The compounds of the present invention include compounds having formula (VII):

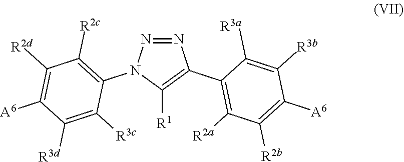

including enantiomers, diastereomers, hydrates, solvates, pharmaceutically acceptable salts, and complexes thereof.

The compounds of the present invention include compounds having formula (VIII):

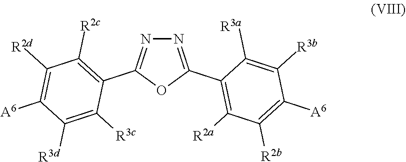

including enantiomers, diastereomers, hydrates, solvates, pharmaceutically acceptable salts, and complexes thereof.

The compounds of the present invention include compounds having formula (IX):

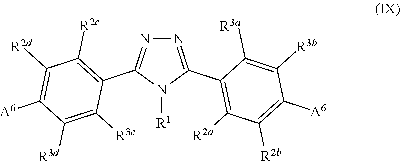

including enantiomers, diastereomers, hydrates, solvates, pharmaceutically acceptable salts, and complexes thereof.

The compounds of the present invention include compounds having formula

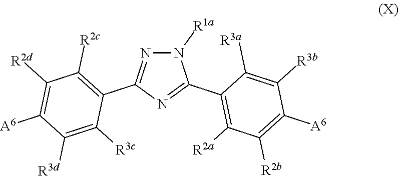

including enantiomers, diastereomers, hydrates, solvates, pharmaceutically acceptable salts, and complexes thereof.

The compounds of the present invention include compounds having formula (XI):

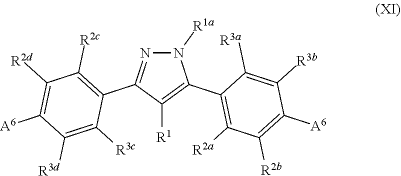

including enantiomers, diastereomers, hydrates, solvates, pharmaceutically acceptable salts, and complexes thereof.

In some embodiments A is CR¹.

In some embodiments A¹ is O.

In some embodiments A¹ is N.

In some embodiments A¹ is NR¹.

In some embodiments A² is N.

In some embodiments A² is C.

In some embodiments A³ is N.

In some embodiments A³ is NR^{1a}.

In some embodiments A⁵ is

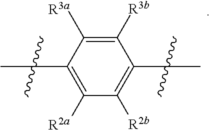

In some embodiments A⁵ is

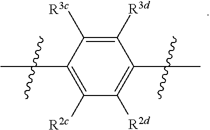

In some embodiments A⁵ is

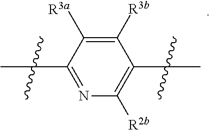

In some embodiments A⁵ is

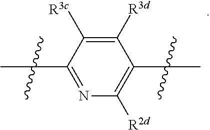

In some embodiments A⁵ is

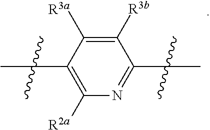

In some embodiments A⁵ is

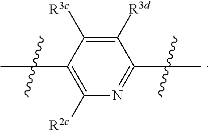

In some embodiments A⁵ is

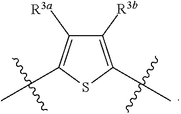

In some embodiments A⁵ is

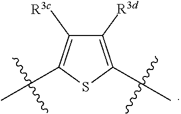

In some embodiments A⁶ is

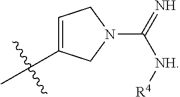

In some embodiments A⁶ is

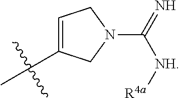

In some embodiments A⁶ is

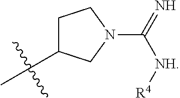

In some embodiments A⁶ is

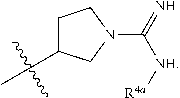

In some embodiments A⁶ is

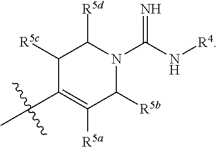

In some embodiments A⁶ is

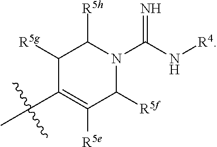

In some embodiments A⁶ is

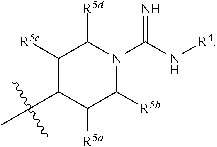

In some embodiments A⁶ is

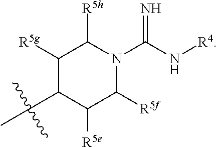

In some embodiments A⁶ is

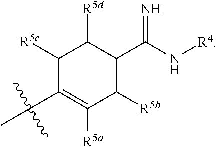

In some embodiments A⁶ is

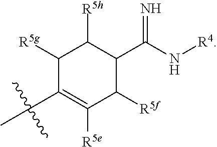

In some embodiments A⁶ is

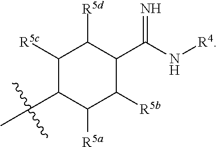

In some embodiments A⁶ is

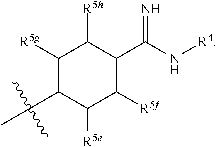

In some embodiments A⁶ is

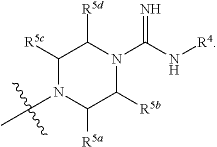

In some embodiments A⁶ is

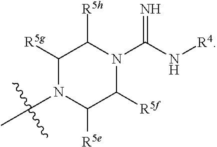

In some embodiments A⁶ is

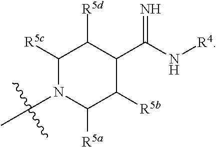

In some embodiments A⁶ is

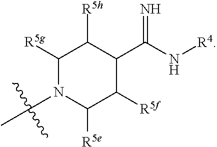

In some embodiments A⁶ is

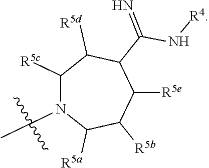

In some embodiments A⁶ is

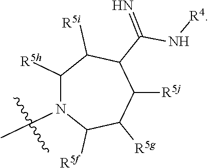

In some embodiments A⁶ is

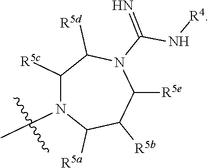

In some embodiments A⁶ is

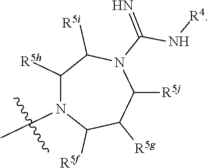

In some embodiments A⁶ is

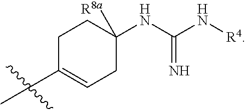

In some embodiments A⁶ is

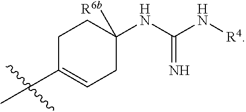

In some embodiments A⁶ is

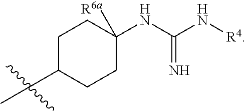

In some embodiments A⁶ is

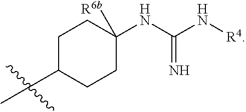

In some embodiments R¹ is hydrogen.

In some embodiments R¹ is C₁₋₈ alkyl.

In some embodiments R¹ is C₃₋₈ branched alkyl.

In some embodiments R¹ is C₃₋₈ cycloalkyl.

In some embodiments R¹ is benzyl.

In some embodiments R¹ is optionally substituted benzyl

In some embodiments R¹ is benzyl that is optionally substituted with a substituent selected from the group consisting of F, Me, OMe, and CF₃.

In some embodiments R¹ is

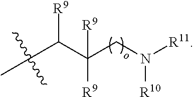

In some embodiments R¹ is

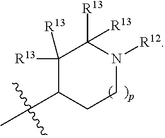

In some embodiments R¹ is

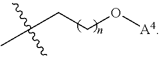

In some embodiments R¹ is

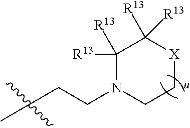

In some embodiments R¹ is selected from the group consisting of

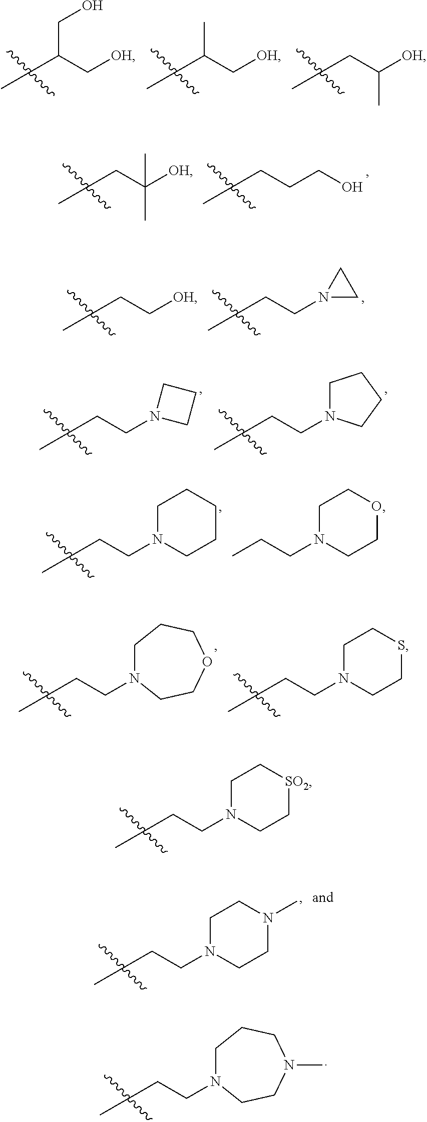

In some embodiments R^{1a} is hydrogen.

In some embodiments R^{1a} is C₁₋₈ alkyl.

In some embodiments R^{1a} is C₃₋₈ branched alkyl.

In some embodiments R^{1a} is C₃₋₈ cycloalkyl.

In some embodiments R^{1a} is benzyl.

In some embodiments R^{1a} is optionally substituted benzyl.

In some embodiments R¹³ is benzyl that is optionally substituted with a substituent selected from the group consisting of F, Me, OMe, and CF₃.

In some embodiments R^{1a} is

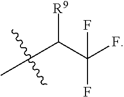

In some embodiments R^{1a} is

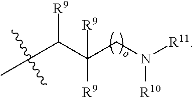

In some embodiments R^{1a} is

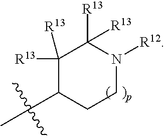

In some embodiments R^{1a} is

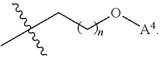

In some embodiments R^{1a} is

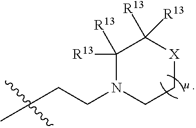

In some embodiments R^{1a} is selected from the group consisting of

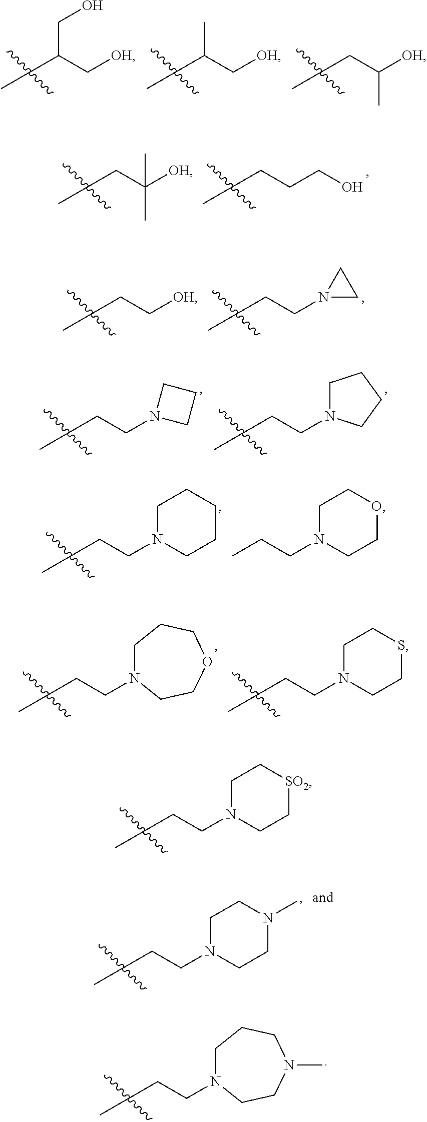

In some embodiments A⁴ is C₁₋₄ alkyl.

In some embodiments A⁴ is C₃₋₅ blenched alkyl.

In some embodiments A⁴ is C₃₋₈ cycloalkyl.

In some embodiments A⁴ is

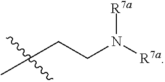

In some embodiments A⁴ is

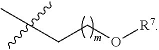

In some embodiments R⁷ is hydrogen.

In some embodiments R⁷ is C₁₋₄ alkyl.

In some embodiments R⁷ is C₃₋₅ branched alkyl.

In some embodiments R^{7a} is C₁₋₄ alkyl.

In some embodiments R^{7a} is C₃₋₈ branched alkyl.

In some embodiments R^{7a} is C₃₋₈ cycloalkyl.

In some embodiments R⁸ is hydrogen.

In some embodiments R⁸ is C₁₋₄ alkyl.

In some embodiments R^{8a} is hydrogen.

In some embodiments R^{8a} is C₁₋₄alkyl.

In some embodiments R^{8a} is

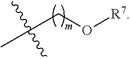

In some embodiments R⁹ is hydrogen.

In some embodiments R⁹ is C₁₋₄alkyl.

In some embodiments R¹⁰ is hydrogen.

In some embodiments R¹⁰ is C₁₋₄ alkyl.

In some embodiments R¹⁰ is C₃₋₈ branched alkyl.

In some embodiments R¹⁰ is C₃₋₈ cycloalkyl.

In some embodiments R¹¹ is C₁₋₄ alkyl.

In some embodiments R¹¹ is C₃₋₈ branched alkyl.

In some embodiments R¹¹ is C₃₋₈ cycloalkyl.

In some embodiments R¹¹ is

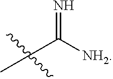 [SMILES: *C(=N)N]

In some embodiments R¹⁰ and R¹¹ are optionally joined to form a heterocyclic ring consisting of three members.

In some embodiments R¹⁰ and R¹¹ are optionally joined to form a heterocyclic ring consisting of four members.

In some embodiments R¹⁰ and R¹¹ are optionally joined to form a heterocyclic ring consisting of five members.

In some embodiments R¹⁰ and R¹¹ are optionally joined to form a heterocyclic ring consisting of six members.

In some embodiments R¹⁰ and R¹¹ are optionally joined to form a heterocyclic ring consisting of seven members.

In some embodiments R¹² is C₁₋₄ alkyl.

In some embodiments R¹² is C₃₋₈ branched alkyl.

In some embodiments R¹² is C₃₋₈ cycloalkyl.

In some embodiments R¹² is

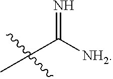 [SMILES: *C(=N)N]

In some embodiments R¹³ is hydrogen.

In some embodiments R¹³ is C₁₋₄ alkyl.

In some embodiments R^{2a} is hydrogen.

In some embodiments R^{2a} is C₁₋₄ alkyl.

In some embodiments R^{2a} is fluorine.

In some embodiments R^{2a} is chlorine.

In some embodiments R^{2a} is C₁₋₄-alkoxy.

In some embodiments R^{2a} is CN.

In some embodiments R^{2a} is CF₃.

In some embodiments R^{2a} is OCF₃.

In some embodiments R^{2b} is hydrogen.

In some embodiments R^{2b} is C₁₋₄ alkyl.

In some embodiments R^{2b} is fluorine.

In some embodiments R^{2b} is chlorine.

In some embodiments R^{2b} is C₁₋₄-alkoxy.

In some embodiments R^{2b} is CN.

In some embodiments R^{2b} is CF₃.

In some embodiments R^{2b} is OCF₃.

In some embodiments R^{2c} is hydrogen.

In some embodiments R^{2c} is C₁₋₄ alkyl.

In some embodiments R^{2c} is fluorine.

In some embodiments R^{2c} is chlorine.

In some embodiments R^{2c} is C₁₋₄-alkoxy.

In some embodiments R^{2c} is CN.

In some embodiments R^{2c} is CF₃.

In some embodiments R^{2c} is OCF₃.

In some embodiments R^{2d} is hydrogen.

In some embodiments R^{2d} is C₁₋₄alkyl.

In some embodiments R^{2d} is fluorine.

In some embodiments R^{2d} is chlorine.

In some embodiments R^{2d} is C₁₋₄-alkoxy.

In some embodiments R^{2d} is CN.

In some embodiments R^{2d} is CF₃.

In some embodiments R^{2d} is OCF₃.

In some embodiments R^{3a} is hydrogen.

In some embodiments R^{3a} is C₁₋₄ alkyl.

In some embodiments R^{3a} is fluorine.

In some embodiments R^{3a} is chlorine.

In some embodiments R^{3a} is C₁₋₄-alkoxy.

In some embodiments R^{3a} is CN.

In some embodiments R^{3a} is CF₃.

In some embodiments R^{3a} is OCF₃.

In some embodiments R^{3b} is hydrogen.

In some embodiments R^{3b} is C₁₋₄ alkyl.

In some embodiments R^{3b} is fluorine.

In some embodiments R^{3b} is chlorine.

In some embodiments R^{3b} is C₁₋₄-alkoxy.

In some embodiments R^{1b} is CN.

In some embodiments R^{3b} is CF₃.

In some embodiments R^{3b} is OCF₃.

In some embodiments R^{3c} is hydrogen.

In some embodiments R^{3c} is C₁₋₄ alkyl.

In some embodiments R^{3c} is fluorine.

In some embodiments R^{3c} is chlorine.

In some embodiments R^{3c} is C₁₋₄-alkoxy.

In some embodiments R^{3c} is CN.

In some embodiments R^{3c} is CF₃.

In some embodiments R^{3c} is OCF₃.

In some embodiments R^{3d} is hydrogen.

In some embodiments R^{3d} is C₁₋₄ alkyl.

In some embodiments R^{3d} is fluorine.

In some embodiments R^{3d} is chlorine.

In some embodiments R^{3d} is C₁₋₄-alkoxy.

In some embodiments R^{3d} is CN.

In some embodiments R^{3d} is CF₃.

In some embodiments R^{3d} is OCF₃.

In some embodiments R⁴ is hydrogen.

In some embodiments R⁴ is C₁₋₄alkyl.

In some embodiments R^{4a} is hydrogen.

In some embodiments R^{4a} is C₁₋₄ alkyl.

In some embodiments R^{5a} is hydrogen.

In some embodiments R^{5a} is C₁₋₄ alkyl.

In some embodiments R^{5b} is hydrogen.

In some embodiments R^{5b} is C₁₋₄ alkyl.

In some embodiments R^{5c} is hydrogen.

In some embodiments R^{5c} is C₁₋₄ alkyl.

In some embodiments R^{5d} is hydrogen.

In some embodiments R^{5d} is C₁₋₄ alkyl.

In some embodiments R^{5e} is hydrogen.

In some embodiments R^{5e} is C₁₋₄ alkyl.

In some embodiments R^{5f} is hydrogen.

In some embodiments R^{5f} is C₁₋₄alkyl.

In some embodiments R^{5g} is hydrogen.

In some embodiments R^{5g} is C₁₋₄alkyl.

In some embodiments R^{5h} is hydrogen.

In some embodiments R^{5h} is C₁₋₄alkyl.

In some embodiments R⁵ⁱ is hydrogen.

In some embodiments R⁵ⁱ is C₁₋₄ alkyl.

In some embodiments R^{5j} is hydrogen.

In some embodiments R^{5j} is C₁₋₄ alkyl.

In some embodiments R^{6a} is hydrogen.

In some embodiments R^{6a} is C₁₋₄alkyl.

In some embodiments R^{6b} is hydrogen.

In some embodiments R^{6b} is C₁₋₄alkyl.

In some embodiments R⁷ is C₁₋₄ alkyl.

In some embodiments R⁷ is C₃₋₅ branched alkyl.

In some embodiments p is 0.

In some embodiments p is 1.

In some embodiments p is 2.

In some embodiments o is 0.

In some embodiments o is 1.

In some embodiments o is 2.

In some embodiments n is 0.

In some embodiments n is 1.

In some embodiments n is 2.

In some embodiments in is 1.

In some embodiments m is 2.

In some embodiments n is 3.

In some embodiments u is 1.

In some embodiments u is 2.

In some embodiments X is NR¹².

In some embodiments X is oxygen.

In some embodiments X is sulfur.

In some embodiments X is SO₂.

Compounds of the present invention include compounds having the formula (XII) or a pharmaceutically acceptable salt form thereof:

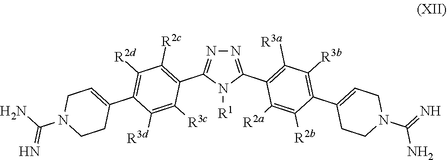

wherein non-limiting examples of R¹, R^{2a}, R^{2b}, R^{3a}, R^{3b}, R^{2c}, R^{2d}, R^{3c}, and R^{3d} are defined herein below in Table 1.

TABLE 1
Exemplary compounds of the formula (XII)

| Entry | R¹ | R^{2a} | R^{2b} | R^{3a} | R^{3b} | R^{2c} | R^{2d} | R^{3c} | R^{3d} |
|---|---|---|---|---|---|---|---|---|---|
| la | Me | H | Cl | H | H | Cl | H | H | H |
| 1b | CH₂CH₂OH | H | Cl | H | H | Cl | H | H | H |
| 1c | CH₂CH₂OMe | H | Cl | H | H | Cl | H | H | H |
| ld | CH₂CH₂OCH2CH₂OCH₂Me | H | Cl | H | H | Cl | H | H | H |
| 2a | Me | Cl | H | H | H | H | H | H | Cl |
| 2b | CH₂CH₂OH | Cl | H | H | H | H | H | H | Cl |
| 2c | CH₂CH₂OMe | Cl | H | H | H | H | H | H | Cl |
| 2d | CH₂CH₂OCH2CH₂OCH₂Me | Cl | H | H | H | H | H | H | Cl |
| 3a | Me | Cl | H | H | H | H | H | H | H |
| 3b | CH₂CH₂OH | Cl | H | H | H | H | H | H | H |
| 3c | CH₂CH₂OMe | Cl | H | H | H | H | H | H | H |
| 3d | CH₂CH₂OCH₂CH₂OCH₂Me | Cl | H | H | H | H | H | H | H |
| 4a | Me | Cl | Cl | H | H | Cl | Cl | H | H |
| 4b | CH₂CH₂OH | Cl | Cl | H | H | Cl | Cl | H | H |
| 4c | CH₂CH₂OMe | Cl | Cl | H | H | Cl | Cl | H | H |
| 4d | CH₂CH₂OCH₂CH₂OCH₂Me | Cl | Cl | H | H | Cl | Cl | H | H |
| 5a | Me | Me | H | H | H | H | H | H | H |
| 5b | CH₂CH₂OH | Me | H | H | H | H | H | H | H |
| 5c | CH₂CH₂OMe | Me | H | H | H | H | H | H | H |
| 5d | CH₂CH₂OCH₂CH₂OCH₂Me | Me | H | H | H | H | H | H | H |
| 6a | Me | H | OMe | H | H | H | H | H | H |
| 6b | CH₂CH₂OH | H | OMe | H | H | H | H | H | H |
| 6c | CH₂CH₂OMe | H | OMe | H | H | H | H | H | H |
| 6d | CH₂CH₂OCH₂CH₂OCH₂Me | H | OMe | H | H | H | H | H | H |
| 7a | Me | OMe | H | H | H | H | H | H | H |
| 7b | CH₂CH₂OH | OMe | H | H | H | H | H | H | H |
| 7c | CH₂CH₂OMe | OMe | H | H | H | H | H | H | H |
| 7d | CH₂CH₂OCH₂CH₂OCH₂Me | OMe | H | H | H | H | H | H | H |
| 8a | Me | OMe | H | H | H | H | OMe | H | H |
| 8b | CH₂CH₂OH | OMe | H | H | H | H | OMe | H | H |
| 8c | CH₂CH₂OMe | OMe | H | H | H | H | OMe | H | H |
| 8d | CH₂CH₂OCH₂CH₂OCH₂Me | OMe | H | H | H | H | OMe | H | H |
| 9a | Me | H | OMe | H | H | OMe | H | H | H |
| 9b | CH₂CH₂OH | H | OMe | H | H | OMe | H | H | H |
| 9c | CH₂CH₂OMe | H | OMe | H | H | OMe | H | H | H |
| 9d | CH₂CH₂OCH₂CH₂OCH₂Me | H | OMe | H | H | OMe | H | H | H |
| 10a | Me | H | CF₃ | H | H | H | H | H | H |
| 10b | CH₂CH₂OH | H | CF₃ | H | H | H | H | H | H |
| 10c | CH₂CH₂OMe | H | CF₃ | H | H | B | H | H | H |
| 10d | CH₂CH₂OCH₂CH₂OCH₂Me | H | CF₃ | H | H | H | H | H | H |
| 1la | Me | H | CF₃ | H | H | H | H | CF₃ | H |
| 11b | CH₂CH₂OH | H | CF₃ | H | H | H | H | CF₃ | H |
| 11c | CH₂CH₂OMe | H | CF₃ | H | H | H | H | CF₃ | H |
| 11d | CH₂CH₂OCH₂CH₂OCH₂Me | H | CF₃ | H | H | H | H | CF₃ | H |
| 12a | Me | CF₃ | H | H | H | H | CF₃ | H | H |
| 12b | CH₂CH₂OH | CF₃ | H | H | H | H | CF₃ | H | H |
| 12c | CH₂CH₂OMe | CF₃ | H | H | H | H | CF₃ | H | H |
| 12d | CH₂CH₂OCH₂CH₂OCH₂Me | CF₃ | H | H | H | H | CF₃ | H | H |

Compounds of the present invention include compounds having the formula (XIII) or a pharmaceutically acceptable salt form thereof:

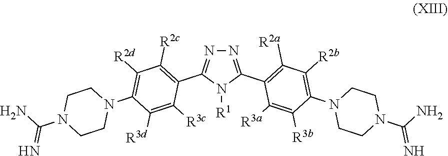

wherein non-limiting examples of R¹, R^{2a}, R^{2b}, R^{3a}, R^{2c}, R^{2d}, R^{3c} and R^{3d} are defined herein below in Table 2.

TABLE 2
Exemplary compounds of the formula (XIII)

| Entry | R¹ | R^{2a} | R^{2b} | R^{3a} | R^{3b} | R^{2c} | R^{2d} | R^{3c} | R^{3d} |
|---|---|---|---|---|---|---|---|---|---|
| 1a | Me | H | F | H | H | H | H | H | H |
| 1b | CH₂CH₂OH | H | F | H | H | H | H | H | H |
| 1c | CH₂CH₂OMe | H | F | H | H | H | H | H | H |
| 1d | CH₂CH₂OCH₂CH₂OCH₂Me | H | F | H | H | H | H | H | H |
| 2a | Me | F | H | H | H | H | H | H | H |
| 2b | CH₂CH₂OH | F | H | H | H | H | H | H | H |
| 2c | CH₂CH₂OMe | F | H | H | H | H | H | H | H |
| 2d | CH₂CH₂OCH₂CH₂OCH₂Me | F | H | H | H | H | H | H | H |
| 3a | Me | H | F | H | H | H | F | H | H |
| 3b | CH₂CH₂OH | H | F | H | H | H | F | H | H |
| 3c | CH₂CH₂OMe | H | F | H | H | H | F | H | H |
| 3d | CH₂CH₂OCH₂CH₂OCH₂Me | H | F | H | H | H | F | H | H |
| 4a | Me | F | H | H | H | F | H | H | H |
| 4b | CH₂CH₂OH | F | H | H | H | F | H | H | H |
| 4c | CH₂CH₂OMe | F | H | H | H | F | H | H | H |
| 4d | CH₂CH₂OCH₂CH₂OCH₂Me | F | H | H | H | F | H | H | H |
| 5a | Me | H | Cl | H | H | H | H | H | H |
| 5b | CH₂CH₂OH | H | Cl | H | H | H | H | H | H |
| 5c | CH₂CH₂OMe | H | Cl | H | H | H | H | H | H |
| 5d | CH₂CH₂OCH₂CH₂OCH₂Me | H | Cl | H | H | H | H | H | H |
| 6a | Me | Cl | H | H | H | H | H | H | H |
| 6b | CH₂CH₂OH | Cl | H | H | H | H | H | H | H |
| 6c | CH₂CH₂OMe | Cl | H | H | H | H | H | H | H |
| 6d | CH₂CH₂OCH₂CH₂OCH₂Me | Cl | H | H | H | H | H | H | H |
| 7a | Me | H | Cl | H | H | H | Cl | H | H |
| 7b | CH₂CH₂OH | H | Cl | H | H | H | Cl | H | H |
| 7c | CH₂CH₂OMe | H | Cl | H | H | H | Cl | H | H |
| 7d | CH₂CH₂OCH₂CH₂OCH₂Me | H | Cl | H | H | H | Cl | H | H |
| 8a | Me | Cl | H | H | H | Cl | H | H | H |
| 8b | CH₂CH₂OH | Cl | H | H | H | Cl | H | H | H |
| 8c | CH₂CH₂OMe | Cl | H | H | H | Cl | H | H | H |
| 8d | CH₂CH₂OCH₂CH₂OCH₂Me | Cl | H | H | H | Cl | H | H | H |
| 9a | Me | H | Me | H | H | H | H | H | H |
| 9b | CH₂CH₂OH | H | Me | H | H | H | H | H | H |
| 9c | CH₂CH₂OMe | H | Me | H | H | H | H | H | H |
| 9d | CH₂CH₂OCH₂CH₂OCH₂Me | H | Me | H | H | H | H | H | H |
| 10a | Me | Me | H | H | H | H | H | H | H |
| 10b | CH₂CH₂OH | Me | H | H | H | H | H | H | H |
| 10c | CH₂CH₂OMe | Me | H | H | H | H | H | H | H |
| 10d | CH₂CH₂OCH₂CH₂OCH₂Me | Me | H | H | H | H | H | H | H |
| 11a | Me | H | M | H | H | H | Me | H | H |
| 11b | CH₂CH₂OH | H | Me | H | H | H | Me | H | H |
| 11c | CH₂CH₂OMe | H | Me | H | H | H | Me | H | H |
| 11d | CH₂CH₂OCH₂CH₂OCH₂Me | H | Me | H | H | H | Me | H | H |
| 12a | Me | Me | H | H | H | Me | H | H | H |
| 12b | CH₂CH₂OH | Me | H | H | H | Me | H | H | H |
| 12c | CH₂CH₂OMe | Me | H | H | H | Me | H | H | H |
| 12d | CH₂CH₂OCH₂CH₂OCH₂Me | Me | H | H | H | Me | H | H | H |
| 13a | Me | H | OMe | H | H | H | H | H | H |
| 13b | CH₂CH₂OH | H | OMe | H | H | H | H | H | H |
| 13c | CH₂CH₂OMe | H | OMe | H | H | H | H | H | H |
| 13d | CH₂CH₂OCH₂CH₂OCH₂Me | H | OMe | H | H | H | H | H | H |
| 14a | Me | OMe | H | H | H | H | H | H | H |
| 14b | CH₂CH₂OH | OMe | H | H | H | H | H | H | H |
| 14c | CH₂CH₂OMe | OMe | H | H | H | H | H | H | H |
| 14d | CH₂CH₂OCH₂CH₂OCH₂Me | OMe | H | H | H | H | H | H | H |
| 15a | Me | H | OMe | H | H | H | OMe | H | H |
| 15b | CH₂CH₂OH | H | OMe | H | H | H | OMe | H | H |
| 15c | CH₂CH₂OMe | H | OMe | H | H | H | OMe | H | H |
| 15d | CH₂CH₂OCH₂CH₂OCH₂Me | H | OMe | H | H | H | OMe | H | H |
| 16a | Me | OMe | H | H | H | OMe | H | H | H |
| 16b | CH₂CH₂OH | OMe | H | H | H | OMe | H | H | H |
| 16c | CH₂CH₂OMe | OMe | H | H | H | OMe | H | H | H |
| 16d | CH₂CH₂OCH₂CH₂OCH₂Me | OMe | H | H | H | OMe | H | H | H |
| 17a | Me | H | CF₃ | H | H | H | H | H | H |
| 176 | CH₂CH₂OH | H | CF₃ | H | H | H | H | H | H |
| 17c | CH₂CH₂OMe | H | CF₃ | H | H | H | H | H | H |
| 17d | CH₂CH₂OCH₂CH₂OCH₂Me | H | CF₃ | H | H | H | H | H | H |
| 18a | Me | CF₃ | H | H | H | H | H | H | H |
| 18b | CH₂CH₂OH | CF₃ | H | H | H | H | H | H | H |
| 18c | CH₂CH₂OMe | CF₃ | H | H | H | H | H | H | H |
| 18d | CH₂CH₂OCH₂CH₂OCH₂Me | CF₃ | H | H | H | H | H | H | H |
| 19a | Me | H | CF₃ | H | H | H | CF₃ | H | H |
| 19b | CH₂CH₂OH | H | CF₃ | H | H | H | CF | H | H |
| 19c | CH₂CH₂OMe | H | CF₃ | H | H | H | CF₃ | H | H |
| 19d | CH₂CH₂OCH₂CH₂OCH₂Me | H | CF₃ | H | H | H | CF₃ | H | H |
| 20a | Me | CF₃ | H | H | H | CF₃ | H | H | H |
| 20b | CH₂CH₂OH | CF₃ | H | H | H | CF₃ | H | H | H |
| 20c | CH₂CH₂OMe | CF₃ | H | H | H | CF₃ | H | H | H |
| 20d | CH₂CH₂OCH₂CH₂OCH₂Me | CF₃ | H | H | H | CF₃ | H | H | H |

Compounds of the present invention include compounds having the formula (XIV) or a pharmaceutically acceptable salt form thereof:

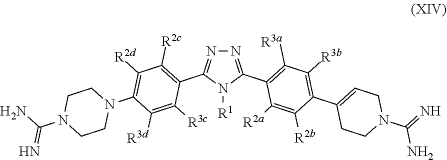

wherein non-limiting examples of R¹, R^{2a}, R^{2b}, R^{3a}, R^{3b}, R^{2c}, R^{2d}, R^{3c}, and R^{3d} are defined herein below in Table 3

TABLE 3
Exemplary compounds of the formula (XIV)

| Entry | R¹ | R^{2a} | R^{2b} | R^{3a} | R^{3b} | R^{2c} | R^{2d} | R^{3c} | R^{3d} |
|---|---|---|---|---|---|---|---|---|---|
| 1a | Me | H | H | H | H | H | H | H | H |
| 1b | CH₂CH₂OH | H | H | H | H | H | H | H | H |
| 1c | CH₂CH₂OMe | H | H | H | H | H | H | H | H |
| 1d | CH₂CH₂OCH₂CH₂OCH₂Me | H | H | H | H | H | H | H | H |
| 2a | Me | H | F | H | H | H | H | H | H |
| 2b | CH₂CH₂OH | H | F | H | H | H | H | H | H |
| 2c | CH₂CH₂OMe | H | F | H | H | H | H | H | H |
| 2d | CH₂CH₂OCH₂CH₂OCH₂Me | H | F | H | H | H | H | H | H |
| 3a | Me | H | H | H | H | H | H | H | H |
| 3b | CH₂CH₂OH | F | H | H | H | H | H | H | H |
| 3c | CH₂CH₂OMe | F | H | H | H | H | H | H | H |
| 3d | CH₂CH₂OCH₂CH₂OCH₂Me | F | H | H | H | H | H | H | H |
| 4a | Me | H | F | H | H | H | F | H | H |
| 4b | CH₂CH₂OH | H | F | H | H | H | F | H | H |
| 4c | CH₂CH₂OMe | H | F | H | H | H | F | H | H |
| 4d | CH₂CH₂OCH₂CH₂OCH₂Me | H | F | H | H | H | F | H | H |
| 5a | Me | F | H | H | H | F | H | H | H |
| 5b | CH₂CH₂OH | F | H | H | H | F | H | H | H |
| 5c | CH₂CH₂OMe | F | H | H | H | F | H | H | H |
| 5d | CH₂CH₂OCH₂CH₂OCH₂Me | F | H | H | H | F | H | H | H |
| 6a | Me | H | Cl | H | H | H | H | H | H |
| 6b | CH₂CH₂OH | H | Cl | H | H | H | H | H | H |
| 6c | CH₂CH₂OMe | H | Cl | H | H | H | H | H | H |
| 6d | CH₂CH₂OCH₂CH₂OCH₂Me | H | Cl | H | H | H | H | H | H |
| 7a | Me | Cl | H | H | H | H | H | H | H |
| 7b | CH₂CH₂OH | Cl | H | H | H | H | H | H | H |
| 7c | CH₂CH₂OMe | Cl | H | H | H | H | H | H | H |
| 7d | CH₂CH₂OCH₂CH₂OCH₂Me | Cl | H | H | H | H | H | H | H |
| 8a | Me | H | Cl | H | H | H | Cl | H | H |
| 8b | CH₂CH₂OH | H | Cl | H | H | H | Cl | H | H |
| 8c | CH₂CH₂OMe | H | Cl | H | H | H | Cl | H | H |
| 8d | CH₂CH₂OCH₂CH₂OCH₂Me | H | Cl | H | H | H | Cl | H | H |
| 9a | Me | Cl | H | H | H | Cl | H | H | H |
| 9b | CH₂CH₂OH | Cl | H | H | H | Cl | H | H | H |
| 9c | CH₂CH₂OMe | Cl | H | H | H | Cl | H | H | H |
| 9d | CH₂CH₂OCH₂CH₂OCH₂Me | Cl | H | H | H | Cl | H | H | H |
| 10a | Me | H | Me | H | H | H | H | H | H |
| 10b | CH₂CH₂OH | H | Me | H | H | H | H | H | H |
| 10c | CH₂CH₂OMe | H | Me | H | H | H | H | H | H |
| 10d | CH₂CH₂OCH₂CH₂OCH₂Me | H | Me | H | H | H | H | H | H |
| 11a | Me | Me | H | H | H | H | H | H | H |
| 11b | CH₂CH₂OH | Me | H | H | H | H | H | H | H |
| 11c | CH₂CH₂OMe | Me | H | H | H | H | H | H | H |
| 11d | CH₂CH₂OCH₂CH₂OCH₂Me | Me | H | H | H | H | H | H | H |
| 12a | Me | H | Me | H | H | H | Me | H | H |
| 12b | CH₂CH₂OH | H | Me | H | H | H | Me | H | H |
| 12c | CH₂CH₂OMe | H | Me | H | H | H | Me | H | H |
| 12d | CH₂CH₂OCH₂CH₂OCH₂Me | H | Me | H | H | H | Me | H | H |
| 13a | Me | Me | H | H | H | Me | H | H | H |
| 13b | CH₂CH₂OH | Me | H | H | H | Me | H | H | H |
| 13c | CH₂CH₂OMe | Me | H | H | H | Me | H | H | H |
| 13d | CH₂CH₂OCH₂CH₂OCH₂Me | Me | H | H | H | Me | H | H | H |
| 14a | Me | H | OMe | H | H | H | H | H | H |
| 14b | CH₂CH₂OH | H | OMe | H | H | H | H | H | H |
| 14c | CH₂CH₂OMe | H | OMe | H | H | H | H | H | H |
| 14d | CH₂CH₂OCH₂CH₂OCH₂Me | H | OMe | H | H | H | H | H | H |
| 15a | Me | OMe | H | H | H | H | H | H | H |
| 15b | CH₂CH₂OH | OMe | H | H | H | H | H | H | H |
| 15c | CH₂CH₂OMe | OMe | H | H | H | H | H | H | H |
| 15d | CH₂CH₂OCH₂CH₂OCH₂Me | OMe | H | H | H | H | H | H | H |
| 16a | Me | H | OMe | H | H | H | OMe | H | H |
| 16b | CH₂CH₂OH | H | OMe | H | H | H | OMe | H | H |
| 16c | CH₂CH₂OMe | H | OMe | H | H | H | OMe | H | H |
| 16d | CH₂CH₂OCH₂CH₂OCH₂Me | H | OMe | H | H | H | OMe | H | H |
| 17a | Me | OMe | H | H | H | OMe | H | H | H |
| 17b | CH₂CH₂OH | OMe | H | H | H | OMe | H | H | H |
| 17c | CH₂CH₂OMe | OMe | H | H | H | OMe | H | H | H |
| 17d | CH₂CH₂OCH₂CH₂OCH₂Me | OMe | H | H | H | OMe | H | H | H |
| 18A | Me | H | CF₃ | H | H | H | H | H | H |
| 18b | CH₂CH₂OH | H | CF₃ | H | H | H | H | H | H |
| 18c | CH₂CH₂OMe | H | CF₃ | H | H | H | H | H | H |
| 18d | CH₂CH₂OCH₂CH₂OCH₂Me | H | CF₃ | H | H | H | H | H | H |
| 19a | Me | CF₃ | H | H | H | H | H | H | H |
| 19b | CH₂CH₂OH | CF₃ | H | H | H | H | H | H | H |
| 19c | CH₂CH₂OMe | CF₃ | H | H | H | H | H | H | H |
| 19d | CH₂CH₂OCH₂CH₂OCH₂Me | CF₃ | H | H | H | H | H | H | H |
| 20a | Me | H | CF₃ | H | H | H | CF₃ | H | H |
| 20b | CH₂CH₂OH | H | CF₃ | H | H | H | CF₃ | H | H |
| 20c | CH₂CH₂OMe | H | CF₃ | H | H | H | CF₃ | H | H |
| 20d | CH₂CH₂OCH₂CH₂OCH₂Me | H | CF₃ | H | H | H | CF₃ | H | H |
| 21a | Me | CF₃ | H | H | H | CF₃ | H | H | H |
| 21b | CH₂CH₂OH | CF₃ | H | H | H | CF₃ | H | H | H |
| 21c | CH₂CH₂OMe | CF₃ | H | H | H | CF₃ | H | H | H |
| 21d | CH₂CH₂OCH₂CH₂OCH₂Me | CF₃ | H | H | H | CF₃ | H | H | H |

Compounds of the present invention include compounds having the formula (XV) or a pharmaceutically acceptable salt form thereof:

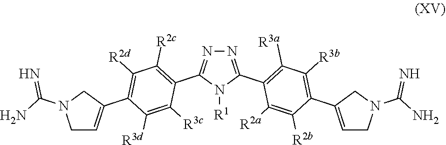

wherein non-limiting examples of R¹, R^{2a}, R^{2b}, R^{3a}, R^{3b}, R^{2c}, R^{2d}, R^{3c}, and R^{3d} are defined herein below in Table 4.

TABLE 4
Exemplary compounds of the formula (XV)

| Entry | R¹ | R^{2a} | R^{2b} | R^{3a} | R^{3b} | R^{2c} | R^{2d} | R^{3c} | R^{3d} |
|---|---|---|---|---|---|---|---|---|---|
| 1a | Me | H | F | H | H | H | H | H | H |
| 1b | CH₂CH₂OH | H | F | H | H | H | H | H | H |
| 1c | CH₂CH₂OMe | H | F | H | H | H | H | H | H |
| Id | CH₂CH₂OCH₂CH₂OCH₂Me | H | F | H | H | H | H | H | H |
| 2a | Me | F | H | H | H | H | H | H | H |
| 2b | CH₂CH₂OH | F | H | H | H | H | H | H | H |
| 2c | CH₂CH₂OMe | F | H | H | H | H | H | H | H |
| 2d | CH₂CH₂OCH₂CH₂OCH₂Me | F | H | H | H | H | H | H | H |
| 3a | Me | H | F | H | H | H | F | H | H |
| 3b | CH₂CH₂OH | H | F | H | H | H | F | H | H |
| 3c | CH₂CH₂OMe | H | F | H | H | H | F | H | H |
| 3d | CH₂CH₂OCH₂CH₂OCH₂Me | H | F | H | H | H | F | H | H |
| 4a | Me | H | Cl | H | H | H | H | H | H |
| 4b | CH₂CH₂OH | H | Cl | H | H | H | H | H | H |
| 4c | CH₂CH₂OMe | H | Cl | H | H | H | H | H | H |
| 4d | CH₂CH₂OCH₂CH₂OCH₂Me | H | Cl | H | H | H | H | H | H |
| 5a | Me | Cl | H | H | H | H | H | H | H |
| 5b | CH₂CH₂OH | Cl | H | H | H | H | H | H | H |
| 5c | CH₂CH₂OMe | Cl | H | H | H | H | H | H | H |
| 5d | CH₂CH₂OCH₂CH₂OCH₂Me | Cl | H | H | H | H | H | H | H |
| 6a | Me | H | Cl | H | H | H | Cl | H | H |
| 6b | CH₂CH₂OH | H | Cl | H | H | H | Cl | H | H |
| 6c | CH₂CH₂OMe | H | Cl | H | H | H | Cl | H | H |
| 6d | CH₂CH₂OCH₂CH₂OCH₂Me | H | Cl | H | H | H | Cl | H | H |
| 7a | Me | Cl | H | H | H | Cl | H | H | H |
| 7b | CH₂CH₂OH | Cl | H | H | H | Cl | H | H | H |
| 7c | CH₂CH₂OMe | Cl | H | H | H | Cl | H | H | H |
| 7d | CH₂CH₂OCH₂CH₂OCH₂Me | Cl | H | H | H | Cl | H | H | H |
| 8ª | Me | H | Me | H | H | H | H | H | H |
| 8b | CH₂CH₂OH | H | Me | H | H | H | H | H | H |
| 8c | CH₂CH₂OMe | H | Me | H | H | H | H | H | H |
| 8d | CH₂CH₂OCH₂CH₂OCH₂Me | H | Me | H | H | H | H | H | H |
| 9a | Me | Me | H | H | H | H | H | H | H |
| 9b | CH₂CH₂OH | Me | H | H | H | H | H | H | H |
| 9c | CH₂CH₂OMe | Me | H | H | H | H | H | H | H |
| 9d | CH₂CH₂OCH₂CH₂OCH₂Me | Me | H | H | H | H | H | H | H |
| 10a | Me | H | Me | H | H | H | Me | H | H |
| 10b | CH₂CH₂OH | H | Me | H | H | H | Me | H | H |
| 10c | CH₂CH₂OMe | H | Me | H | H | H | Me | H | H |
| 10d | CH₂CH₂OCH₂CH₂OCH₂Me | H | Me | H | H | H | Me | H | H |
| 11a | Me | Me | H | H | H | Me | H | H | H |
| 11b | CH₂CH₂OH | Me | H | H | H | Me | H | H | H |
| 11c | CH₂CH₂OMe | Me | H | H | H | Me | H | H | H |
| 11d | CH₂CH₂OCH₂CH₂OCH₂Me | Me | H | H | H | Me | H | H | H |
| 12a | Me | H | OMe | H | H | H | H | H | H |
| 12b | CH₂CH₂OH | H | OMe | H | H | H | H | H | H |
| 12c | CH₂CH₂OMe | H | OMe | H | H | H | H | H | H |
| 12d | CH₂CH₂OCH₂CH₂OCH₂Me | H | OMe | H | H | H | H | H | H |
| 13a | Me | OMe | H | H | H | H | H | H | H |
| 13b | CH₂CH₂OH | OMe | H | H | H | H | H | H | H |
| 13c | CH₂CH₂OMe | OMe | H | H | H | H | H | H | H |
| 13d | CH₂CH₂OCH₂CH₂OCH₂Me | OMe | H | H | H | H | H | H | H |
| 14a | Me | H | OMe | H | H | H | OMe | H | H |
| 14b | CH₂CH₂OH | H | OMe | H | H | H | OMe | H | H |
| 14c | CH₂CH₂OMe | H | OMe | H | H | H | OMe | H | H |
| 14d | CH₂CH₂OCH₂CH₂OCH₂Me | H | OMe | H | H | H | OMe | H | H |
| 15a | Me | OMe | H | H | H | OMe | H | H | H |
| 15b | CH₂CH₂OH | OMe | H | H | H | OMe | H | H | H |
| 15c | CH₂CH₂OMe | OMe | H | H | H | OMe | H | H | H |
| 15d | CH₂CH₂OCH₂CH₂OCH₂Me | OMe | H | H | H | OMe | H | H | H |
| 16a | Me | CF₃ | H | H | H | H | H | H | H |
| 16b | CH₂CH₂OH | CF₃ | H | H | H | H | H | H | H |
| 16c | CH₂CH₂OMe | CF₃ | H | H | H | H | H | H | H |
| 16d | CH₂CH₂OCH₂CH₂OCH₂Me | CF₃ | H | H | H | H | H | H | H |
| 17a | Me | H | CF₃ | H | H | H | CF₃ | H | H |
| 17b | CH₂CH₂OH | H | CF₃ | H | H | H | CF₃ | H | H |
| 17c | CH₂CH₂OMe | H | CF₃ | HH | H | H | CF₃ | H | H |
| 17d | CH₂CH₂OCH₂CH₂OCH₂Me | H | CF | H | H | H | CF₃ | H | H |
| 18a | Me | CF₃ | H | H | H | CF₃ | H | H | H |
| 18b | CH₂CH₂OH | CF₃ | H | H | H | CF₃ | H | H | H |
| 18c | CH₂CH₂OMe | CF₃ | H | H | H | CF₃ | H | H | H |
| 18d | CH₂CH₂OCH₂CH₂OCH₂Me | CF₃ | H | H | H | CF₃ | H | H | H |

Compounds of the present invention include compounds having the formula (XVI) or a pharmaceutically acceptable salt form thereof:

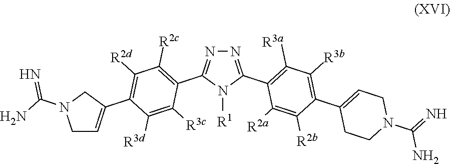

wherein non-limiting examples of R¹, R^{2a}, R^{2b}, R^{3a}, R^{3b}, R^{2c}, R^{2d}, R^{3c}, and R^{3d} are defined herein below in Table 5.

TABLE 5
Exemplary compounds of the formula (XVI)

| Entry | R¹ | R^{2a} | R^{2b} | R^{3a} | R^{3b} | R^{2c} | R^{2d} | R^{3c} | R^{3d} |
|---|---|---|---|---|---|---|---|---|---|
| 1a | Me | H | H | H | H | H | H | H | H |
| 1b | CH₂CH₂OH | H | H | H | H | H | H | H | H |
| 1c | CH₂CH₂OMe | H | H | H | H | H | H | H | H |
| 1d | CH₂CH₂OCH₂CH₂OCH₂Me | H | H | H | H | H | H | H | H |
| 2a | Me | H | F | H | H | H | H | H | H |
| 2b | CH₂CH₂OH | H | F | H | H | H | H | H | H |
| 2c | CH₂CH₂OMe | H | F | H | H | H | H | H | H |
| 2d | CH₂CH₂OCH₂CH₂OCH₂Me | H | F | H | H | H | H | H | H |
| 3a | Me | F | H | H | H | H | H | H | H |
| 3b | CH₂CH₂OH | F | H | H | H | H | H | H | H |
| 3c | CH₂CH₂OMe | F | H | H | H | H | H | H | H |
| 3d | CH₂CH₂OCH₂CH₂OCH₂Me | F | H | H | H | H | H | H | H |
| 4a | Me | H | F | H | H | H | F | H | H |
| 4b | CH₂CH₂OH | H | F | H | H | H | F | H | H |
| 4c | CH₂CH₂OMe | H | F | H | H | H | F | H | H |
| 4d | CH₂CH₂OCH₂CH₂OCH₂Me | H | F | H | H | H | F | H | H |
| 5a | Me | F | H | H | H | F | H | H | H |
| 5b | CH₂CH₂OH | F | H | H | H | F | H | H | H |
| 5c | CH₂CH₂OMe | F | H | H | H | F | H | H | H |
| 5d | CH₂CH₂OCH₂CH₂OCH₂Me | F | H | H | H | F | H | H | H |
| 6a | Me | H | Cl | H | H | H | H | H | H |
| 6b | CH₂CH₂OH | H | Cl | H | H | H | H | H | H |
| 6c | CH₂CH₂OMe | H | Cl | H | H | H | H | H | H |
| 6d | CH₂CH₂OCH₂CH₂OCH₂Me | H | Cl | H | H | H | H | H | H |
| 7a | Me | Cl | H | H | H | H | H | H | H |
| 7b | CH₂CH₂OH | Cl | H | H | H | H | H | H | H |
| 7c | CH₂CH₂OMe | Cl | H | H | H | H | H | H | H |
| 7d | CH₂CH₂OCH₂CH₂OCH₂Me | Cl | H | H | H | H | H | H | H |
| 8a | Me | H | Cl | H | H | H | Cl | H | H |
| 8b | CH₂CH₂OH | H | Cl | H | H | H | Cl | H | H |
| 8c | CH₂CH₂OMe | H | Cl | H | H | H | Cl | H | H |
| 8d | CH₂CH₂OCH₂CH₂OCH₂Me | H | Cl | H | H | H | Cl | H | H |
| 9a | Me | Cl | H | H | H | Cl | H | H | H |
| 9b | CH₂CH₂OH | Cl | H | H | H | Cl | H | H | H |
| 9c | CH₂CH₂OMe | Cl | H | H | H | Cl | H | H | H |
| 9d | CH₂CH₂OCH₂CH₂OCH₂Me | Cl | H | H | H | Cl | H | H | H |
| 10a | Me | H | Me | H | H | H | H | H | H |
| 10b | CH₂CH₂OH | H | Me | H | H | H | H | H | H |
| 10c | CH₂CH₂OMe | H | Me | H | H | H | H | H | H |
| 10d | CH₂CH₂OCH₂CH₂OCH₂Me | H | Me | H | H | H | H | H | H |
| 11a | Me | Me | H | H | H | H | H | H | H |
| 11b | CH₂CH₂OH | Me | H | H | H | H | H | H | H |
| 11c | CH₂CH₂OMe | Me | H | H | H | H | H | H | H |
| 11d | CH₂CH₂OCH₂CH₂OCH₂Me | Me | H | H | H | H | H | H | H |
| 12a | Me | H | Me | H | H | H | Me | H | H |
| 12b | CH₂CH₂OH | H | Me | H | H | H | Me | H | H |
| 12c | CH₂CH₂OMe | H | Me | H | H | H | Me | H | H |
| 12d | CH₂CH₂OCH₂CH₂OCH₂Me | H | Me | H | H | H | Me | H | H |
| 13a | Me | Me | H | H | H | Me | H | H | H |
| 13b | CH₂CH₂OH | Me | H | H | H | Me | H | H | H |
| 13c | CH₂CH₂OMe | Me | H | H | H | Me | H | H | H |
| 13d | CH₂CH₂OCH₂CH₂OCH₂Me | Me | H | H | H | Me | H | H | H |
| 14a | Me | H | OMe | H | H | H | H | H | H |
| 14b | CH₂CH₂OH | H | OMe | H | H | H | H | H | H |
| 14c | CH₂CH₂OMe | H | OMe | H | H | H | H | H | H |
| 14d | CH₂CH₂OCH₂CH₂OCH₂Me | H | OMe | H | H | H | H | H | H |
| 15a | Me | OMe | H | H | H | H | H | H | H |
| 15b | CH₂CH₂OH | OMe | H | H | H | H | H | H | H |
| 15c | CH₂CH₂OMe | OMe | H | H | H | H | H | H | H |
| 15d | CH₂CH₂OCH₂CH₂OCH₂Me | OMe | H | H | H | H | H | H | H |
| 16a | Me | H | OMe | H | H | H | OMe | H | H |
| 16b | CH₂CH₂OH | H | OMe | H | H | H | OMe | H | H |
| 16c | CH₂CH₂OMe | H | OMe | H | H | H | OMe | H | H |
| 16d | CH₂CH₂OCH₂CH₂OCH₂Me | H | OMe | H | H | H | OMe | H | H |
| 17a | Me | OMe | H | H | H | OMe | H | H | H |
| 17b | CH₂CH₂OH | OMe | H | H | H | OMe | H | H | H |
| 17c | CH₂CH₂OMe | OMe | H | H | H | OMe | H | H | H |
| 17d | CH₂CH₂OCH₂CH₂OCH₂Me | OMe | H | H | H | OMe | H | H | H |
| 18a | Me | H | CF₃ | H | H | H | H | H | H |
| 18b | CH₂CH₂OH | H | CF₃ | H | H | H | H | H | H |
| 18c | CH₂CH₂OMe | H | CF₃ | H | HI | H | H | H | H |
| 18d | CH₂CH₂OCH₂CH₂OCH₂Me | H | CF₃ | H | H | H | H | H | H |
| 19a | Me | CF₃ | H | H | H | H | H | H | H |
| 19b | CH₂CH₂OH | CF₃ | H | H | H | H | H | H | H |
| 19c | CH₂CH₂OMe | CF | H | H | H | H | H | H | H |
| 19d | CH₂CH₂OCH₂CH₂OCH₂Me | CF₃ | H | H | H | H | H | H | H |
| 20a | Me | H | CF₃ | H | H | H | CF₃ | H | H |
| 20b | CH₂CH₂OH | H | CF₃ | H | H | H | CF₃ | H | H |
| 20c | CH₂CH₂OMe | H | CF₃ | H | H | H | CF₃ | H | H |
| 20d | CH₂CH₂OCH₂CH₂OCH₂Me | H | CF₃ | H | H | H | CF₃ | H | H |
| 21a | Me | CF₃ | H | H | H | CF₃ | H | H | H |
| 21b | CH₂CH₂OH | CF₃ | H | H | H | CF₃ | H | H | H |
| 21c | CH₂CH₂OMe | CF₃ | H | H | H | CF₃ | H | H | H |
| 21d | CH₂CH₂OCH₂CH₂OCH₂Me | CF₃ | H | H | H | CF₃ | H | H | H |

Compounds of the present invention include compounds having the formula (XVII) or a pharmaceutically acceptable salt form thereof:

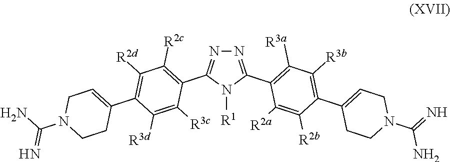

wherein non-limiting examples of R¹, R^{2a}, R^{2b}, R^{3a}, R^{3b}, R^{2c}, R^{2d}, R^{3c}, and R^{3d} are defined herein below in Table 6.

TABLE 6
Exemplary compounds of the formula (XVII)

| Entry | R¹ | R^{2a} | R^{2b} | R^{3a} | R^{3b} | R^{2c} | R^{2d} | R^{3c} | R^{3d} |
|---|---|---|---|---|---|---|---|---|---|
| 1 | Ethyl | H | F | H | H | H | H | H | H |
| 2 | Ethyl | F | H | H | H | H | H | H | H |
| 3 | Ethyl | H | F | H | H | H | F | H | H |
| 4 | Ethyl | F | H | H | H | F | H | H | H |
| 5 | Isopropyl | H | F | H | H | H | H | H | H |
| 6 | Isopropyl | F | H | H | H | H | H | H | H |
| 7 | Isopropyl | H | F | H | H | H | F | H | H |
| 8 | Cyclopropyl | H | F | H | H | H | H | H | H |
| 9 | Cyclopropyl | F | H | H | H | H | H | H | H |
| 10 | 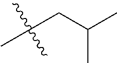 [SMILES: *CC(C)C] | H | H | H | H | H | H | H | H |
| 11 | 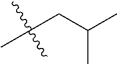 [SMILES: *CC(C)C] | H | F | H | H | H | H | H | H |
| 12 | 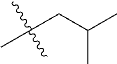 [SMILES: *CC(C)C] | F | H | H | H | H | H | H | H |
| 13 | 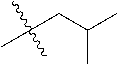 [SMILES: *CC(C)C] | H | F | H | H | H | F | H | H |
| 14 | 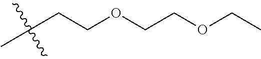 [SMILES: *CCOCCOCC] | H | H | H | H | H | H | H | H |
| 15 | 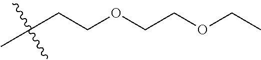 [SMILES: *CCOCCOCC] | H | F | H | H | H | H | H | H |
| 16 | 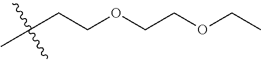 [SMILES: *CCOCCOCC] | F | H | H | H | H | H | H | H |
| 17 | 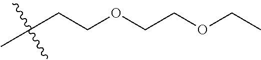 [SMILES: *CCOCCOCC] | H | F | H | H | H | F | H | H |
| 18 | n-octyl | H | H | H | H | H | H | H | H |
| 19 | n-octyl | H | F | H | H | H | H | H | H |
| 20 | n-octyl | F | H | H | H | H | H | H | H |
| 21 | n-octyl | H | F | H | H | H | F | H | H |
| 22 | Benzyl | H | F | H | H | H | H | H | H |
| 23 | Benzyl | H | F | H | H | H | F | H | H |
| 24 | CH₂CH₂OH | H | H | H | H | H | H | H | H |
| 25 | CH₂CH₂OH | H | F | H | H | H | H | H | H |
| 26 | CH₂CH₂OH | F | H | H | H | H | H | H | H |
| 27 | CH₂CH₂OH | H | F | H | H | H | F | H | H |
| 28 | CH₂CH₂OMe | H | H | H | H | H | H | H | H |
| 29 | CH₂CH₂OMe | H | F | H | H | H | H | H | H |
| 30 | CH₂CH₂OMe | F | H | H | H | H | H | H | H |
| 31 | CH₂CH₂OMe | H | F | H | H | H | F | H | H |

Compounds of the present invention include compounds having the formula (XVIII) or a pharmaceutically acceptable salt form thereof:

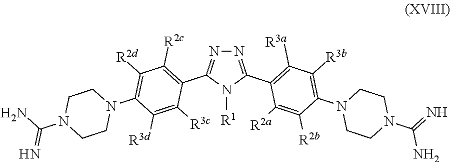

wherein non-limiting examples of R¹, R^{2a}, R^{2b}, R^{3a}, R^{3b}, R^{2c}, R^{2d}, R^{3c}, and R^{3d} are defined herein below in Table 7.

TABLE 7
Exemplary compounds of the formula (XVIII)

| Entry | R¹ | R^{2a} | R^{2b} | R^{3a} | R^{3b} | R^{2c} | R^{2d} | R^{3c} | R^{3d} |
|---|---|---|---|---|---|---|---|---|---|
| 1 | Ethyl | H | H | H | H | H | H | H | H |
| 2 | Ethyl | H | F | H | H | H | H | H | H |
| 3 | Ethyl | F | H | H | H | H | H | H | H |
| 4 | Ethyl | H | F | H | H | H | F | H | H |
| 5 | Ethyl | F | H | H | H | F | H | H | H |
| 6 | Isopropyl | H | H | H | H | H | H | H | H |
| 7 | Isopropyl | F | H | H | H | F | H | H | H |
| 8 | Isopropyl | H | F | H | H | H | H | H | H |
| 9 | Isopropyl | F | H | H | H | H | H | H | H |
| 10 | Isopropyl | H | F | H | H | H | F | H | H |
| 11 | Cyclopropyl | H | H | H | H | H | H | H | H |
| 12 | Cyclopropyl | H | F | H | H | H | F | H | H |
| 13 | Cyclopropyl | F | H | H | H | F | H | H | H |
| 14 | Cyclopropyl | H | F | H | H | H | H | H | H |
| 15 | Cyclopropyl | F | H | H | H | H | H | H | H |
| 16 | 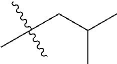 [SMILES: *CC(C)C] | F | H | H | H | F | H | H | H |
| 17 | 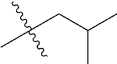 [SMILES: *CC(C)C] | H | H | H | H | H | H | H | H |
| 18 | 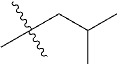 [SMILES: *CC(C)C] | H | F | H | H | H | H | H | H |
| 19 | 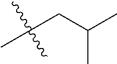 [SMILES: *CC(C)C] | F | H | H | H | H | H | H | H |
| 20 | 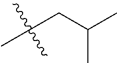 [SMILES: *CC(C)C] | H | F | H | H | H | F | H | H |
| 21 | 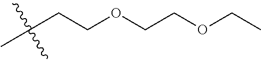 [SMILES: *CCOCCOCC] | F | H | H | H | F | H | H | h |
| 22 | 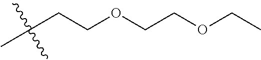 [SMILES: *CCOCCOCC] | H | H | H | H | H | H | H | h |
| 23 | 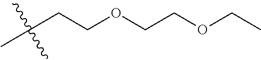 [SMILES: *CCOCCOCC] | H | F | H | H | H | H | H | H |
| 24 | 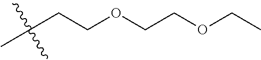 [SMILES: *CCOCCOCC] | F | H | H | H | H | H | H | H |
| 25 | 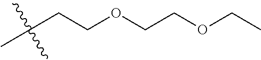 [SMILES: *CCOCCOCC] | H | F | H | H | H | F | H | H |
| 26 | n-Octyl | F | H | H | H | F | H | H | H |
| 27 | n-Octyl | H | H | H | H | H | H | H | H |
| 28 | n-Octyl | H | F | H | H | H | H | H | H |
| 29 | n-Octyl | F | H | H | H | H | H | H | H |
| 30 | n-Octyl | H | F | H | H | H | F | H | H |
| 31 | Benzyl | H | H | H | H | H | H | H | H |
| 32 | Benzyl | F | H | H | H | F | H | H | H |
| 33 | Benzyl | F | H | H | H | H | H | H | H |
| 34 | Benzyl | H | F | H | H | H | H | H | H |
| 35 | Benzyl | H | F | H | H | H | F | H | H |
| 36 | CH₂CH₂OH | H | H | H | H | H | H | H | H |
| 37 | CH₂CH₂OH | F | H | H | H | F | H | H | H |
| 38 | CH₂CH₂OH | F | H | H | H | H | H | H | H |
| 39 | CH₂CH₂OH | H | F | H | H | H | H | H | H |
| 40 | CH₂CH₂OH | H | F | H | H | H | F | H | H |
| 41 | CH₂CH₂OMe | H | H | H | H | H | H | H | H |
| 42 | CH₂CH₂OMe | F | H | H | H | F | H | H | H |
| 42 | CH₂CH₂OMe | F | H | H | H | H | H | H | H |
| 44 | CH₂CH₂OMe | H | F | H | H | H | H | H | H |
| 45 | CH₂CH₂OMe | H | F | H | H | H | F | H | H |

Compounds of the present invention include compounds having the formula (XIX) or a pharmaceutically acceptable salt form thereof:

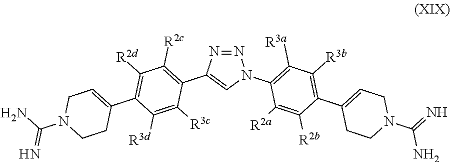

wherein non-limiting examples of R^{2a}, R^{2b}, R^{3a}, R^{3b}, R^{2c}, R^{2d}, R^{3c}, and R^{3d} are defined herein below in Table 8.

TABLE 8
Exemplary compounds of the formula (XIX)

| Entry | R^{2ª} | R^{2b} | R^{3a} | R^{3b} | R^{2c} | R^{2d} | R^{3c} | R^{3d} |
|---|---|---|---|---|---|---|---|---|
| 1 | H | F | H | H | H | H | H | H |
| 2 | F | H | H | H | H | H | H | H |
| 3 | H | H | H | H | F | H | H | H |
| 4 | H | H | H | H | H | F | H | H |
| 5 | H | Cl | H | H | H | H | H | H |
| 6 | Cl | H | H | H | H | H | H | H |
| 7 | H | H | H | H | Cl | H | H | H |
| 8 | H | H | H | H | H | Cl | H | H |
| 9 | H | OMe | H | H | H | H | H | H |
| 10 | OMe | H | H | H | H | H | H | H |
| 11 | H | H | H | H | OMe | H | H | H |
| 12 | H | H | H | H | H | OMe | H | H |
| 13 | H | CF₃ | H | H | H | H | H | H |
| 14 | CF₃ | H | H | H | H | H | H | H |
| 15 | H | H | H | H | CF₃ | H | H | H |
| 16 | H | H | H | H | H | CF₃ | H | H |

Compounds of the present invention include compounds having the formula (XX) or a pharmaceutically acceptable salt form thereof:

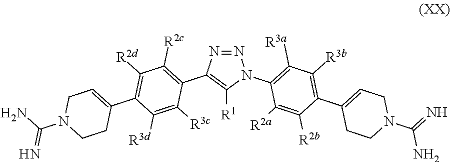

wherein non-limiting examples of R¹, R^{2a}, R^{2b}, R^{3a}, R^{3b}, R^{2c}, R^{2d}, R^{3c}, and R^{3d} are defined herein below in Table 9.

TABLE 9
Exemplary compounds of the formula (XX)

| Entry | R¹ | R^{2ª} | R^{2b} | R^{3a} | R^{3b} | R^{2c} | R^{2d} | R^{3c} | R^{3d} |
|---|---|---|---|---|---|---|---|---|---|
| 1 | Me | H | F | H | H | H | H | H | H |
| 2 | Me | F | H | H | H | H | H | H | H |
| 3 | Me | H | H | H | H | F | H | H | H |
| 4 | Me | H | H | H | H | H | F | H | H |
| 5 | Me | H | Cl | H | H | H | H | H | H |
| 6 | Me | Cl | H | H | H | H | H | H | H |
| 7 | Me | H | H | H | H | Cl | H | H | H |
| 8 | Me | H | H | H | H | H | Cl | H | H |
| 9 | Me | H | H | H | H | Me | H | H | H |
| 10 | Me | H | H | H | H | H | Me | H | H |
| 11 | Me | H | OMe | H | H | H | H | H | H |
| 12 | Me | OMe | H | H | H | H | H | H | H |
| 13 | Me | H | H | H | H | OMe | H | H | H |
| 14 | Me | H | H | H | H | H | OMe | H | H |
| 15 | Me | H | CF₃ | H | H | H | H | H | H |
| 16 | Me | CF₃ | H | H | H | H | H | H | H |
| 17 | Me | H | H | H | H | CF₃ | H | H | H |
| 18 | Me | H | H | H | H | H | CF₃ | H | H |
| 19 | Ethyl | H | H | H | H | H | H | H | H |
| 20 | Ethyl | H | F | H | H | H | H | H | H |
| 21 | Ethyl | F | H | H | H | H | H | H | H |
| 22 | Ethyl | H | H | H | H | F | H | H | H |
| 23 | Ethyl | H | H | H | H | H | F | H | H |
| 24 | Ethyl | H | Cl | H | H | H | H | H | H |
| 25 | Ethyl | Cl | H | H | H | H | H | H | H |
| 26 | Ethyl | H | H | H | H | Cl | H | H | H |
| 27 | Ethyl | H | H | H | H | H | Cl | H | H |
| 28 | Ethyl | H | Me | H | H | H | H | H | H |
| 29 | Ethyl | Me | H | H | H | H | H | H | H |
| 30 | Ethyl | H | H | H | H | Me | H | H | H |
| 31 | Ethyl | H | H | H | H | H | Me | H | H |
| 32 | Ethyl | H | OMe | H | H | H | H | H | H |
| 33 | Ethyl | OMe | H | H | H | H | H | H | H |
| 34 | Ethyl | H | H | H | H | OMe | H | H | H |
| 35 | Ethyl | H | H | H | H | H | OMe | H | H |
| 36 | Ethyl | H | CF₃ | H | H | H | H | H | H |
| 37 | Ethyl | CF₃ | H | H | H | H | H | H | H |
| 38 | Ethyl | H | H | H | H | CF₃ | H | H | H |
| 39 | Ethyl | H | H | H | H | H | CF₃ | H | H |
| 40 | Isopropyl | H | H | H | H | H | H | H | H |
| 41 | Isopropyl | H | F | H | H | H | H | H | H |
| 42 | Isopropyl | F | H | H | H | H | H | H | H |
| 43 | Isopropyl | H | H | H | H | F | H | H | H |
| 44 | Isopropyl | H | H | H | H | H | F | H | H |
| 45 | Isopropyl | H | Cl | H | H | H | H | H | H |
| 46 | Isopropyl | Cl | H | H | H | H | H | H | H |
| 47 | Isopropyl | H | H | H | H | Cl | H | H | H |
| 48 | Isopropyl | H | H | H | H | H | Cl | H | H |
| 49 | Isopropyl | H | Me | H | H | H | H | H | H |
| 50 | Isopropyl | Me | H | H | H | H | H | H | H |
| 51 | Isopropyl | H | H | H | H | Me | H | H | H |
| 52 | Isopropyl | H | H | H | H | H | Me | H | H |
| 53 | Isopropyl | H | OMe | H | H | H | H | H | H |
| 54 | Isopropyl | OMe | H | H | H | H | H | H | H |
| 55 | Isopropyl | H | H | H | H | OMe | H | H | H |
| 56 | Isopropyl | H | H | H | H | H | OMe | H | H |
| 57 | Isopropyl | H | CF₃ | H | H | H | H | H | H |
| 58 | Isopropyl | CF₃ | H | H | H | H | H | H | H |
| 59 | Isopropyl | H | H | H | H | CF₃ | H | H | H |
| 60 | Isopropyl | H | H | H | H | H | CF₃ | H | H |

Compounds of the present invention include compounds having the formula (XXI) or a pharmaceutically acceptable salt form thereof:

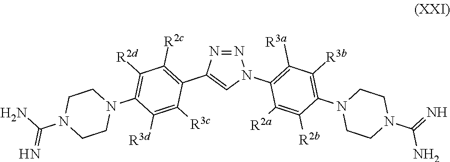

wherein non-limiting examples of R^{2a}, R^{2b}, R^{3a}, R^{3b}, R^{2c}, R^{2d}, R^{3c}, and R^{3d} are defined herein below in Table 10.

TABLE 10
Exemplary compounds of the formula (XXI)

| Entry | R^{2ª} | R^{2b} | R^{3a} | R^{3b} | R^{2c} | R^{2d} | R^{3c} | R^{3d} |
|---|---|---|---|---|---|---|---|---|
| 1 | H | H | H | H | H | H | H | H |
| 2 | H | F | H | H | H | H | H | H |
| 3 | F | H | H | H | H | H | H | H |
| 4 | H | H | H | H | F | H | H | H |
| 5 | H | H | H | H | H | F | H | H |
| 6 | H | Cl | H | H | H | H | H | H |
| 7 | Cl | H | H | H | H | H | H | H |
| 8 | H | H | H | H | Cl | H | H | H |
| 9 | H | H | H | H | H | Cl | H | H |
| 10 | H | OMe | H | H | H | H | H | H |
| 11 | OMe | H | H | H | H | H | H | H |
| 12 | H | H | H | H | OMe | H | H | H |
| 13 | H | H | H | H | H | OMe | H | H |
| 14 | H | CF₃ | H | H | H | H | H | H |
| 15 | CF₃ | H | H | H | H | H | H | H |
| 16 | H | H | H | H | CF₃ | H | H | H |
| 17 | H | H | H | H | H | CF₃ | H | H |

Compounds of the present invention include compounds having the formula (XXII) or a pharmaceutically acceptable salt form thereof:

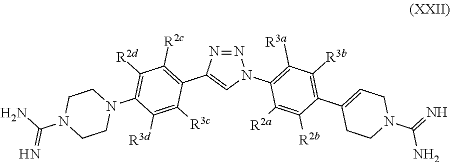

wherein non-limiting examples of R^{2a}, R^{2b}, R^{3a}, R^{3b}, R^{2c}, R^{2d}, R^{3c}, and R^{3d} are defined herein below in Table 11.

TABLE 11
Exemplary compounds of the formula (XXII)

| Entry | R^{2ª} | R^{2b} | R^{3a} | R^{3b} | R^{2c} | R^{2d} | R^{3c} | R^{3d} |
|---|---|---|---|---|---|---|---|---|
| 1 | H | H | H | H | H | H | H | H |
| 2 | H | F | H | H | H | H | H | H |
| 3 | F | H | H | H | H | H | H | H |
| 4 | H | H | H | H | F | H | H | H |
| 5 | H | H | H | H | H | F | H | H |
| 6 | H | Cl | H | H | H | H | H | H |
| 7 | Cl | H | H | H | H | H | H | H |
| 8 | H | H | H | H | Cl | H | H | H |
| 9 | H | H | H | H | H | Cl | H | H |
| 10 | H | OMe | H | H | H | H | H | H |
| 11 | OMe | H | H | H | H | H | H | H |
| 12 | H | H | H | H | OMe | H | H | H |
| 13 | H | H | H | H | H | OMe | H | H |
| 14 | H | CF₃ | H | H | H | H | H | H |
| 15 | CF₃ | H | H | H | H | H | H | H |
| 16 | H | H | H | H | CF₃ | H | H | H |
| 17 | H | H | H | H | H | CF₃ | H | H |

Compounds of the present invention include compounds having the formula (XXIII) or a pharmaceutically acceptable salt form thereof:

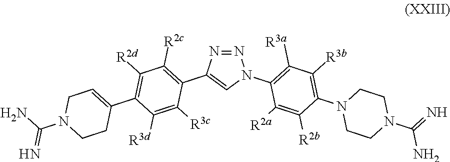

wherein non-limiting examples of R^{2a}, R^{2b}, R^{3a}, R^{3b}, R^{2c}, R^{2d}, R^{3c}, and R^{3d} are defined herein below in Table 12.

TABLE 12
Exemplary compounds of the formula (XXIII)

| Entry | R^{2ª} | R^{2b} | R^{3a} | R^{3b} | R^{2c} | R^{2d} | R^{3c} | R^{3d} |
|---|---|---|---|---|---|---|---|---|
| 1 | H | H | H | H | H | H | H | H |
| 2 | H | F | H | H | H | H | H | H |
| 3 | F | H | H | H | H | H | H | H |
| 4 | H | H | H | H | F | H | H | H |
| 5 | H | H | H | H | H | F | H | H |
| 6 | H | Cl | H | H | H | H | H | H |
| 7 | Cl | H | H | H | H | H | H | H |
| 8 | H | H | H | H | Cl | H | H | H |
| 9 | H | H | H | H | H | Cl | H | H |
| 10 | H | OMe | H | H | H | H | H | H |
| 11 | OMe | H | H | H | H | H | H | H |
| 12 | H | H | H | H | OMe | H | H | H |
| 13 | H | H | H | H | H | OMe | H | h |
| 14 | H | CF₃ | H | H | H | H | H | H |
| 15 | CF₃ | H | H | H | H | H | H | H |
| 16 | H | H | H | H | CF₃ | H | H | H |
| 17 | H | H | H | H | H | CF₃ | H | H |

Compounds of the present invention include compounds having the formula (XXIV) or a pharmaceutically acceptable salt form thereof:

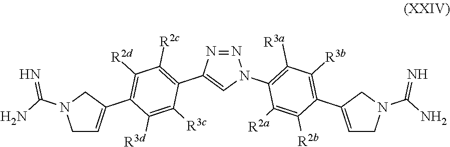

wherein non-limiting examples of R^{2a}, R^{2b}, R^{3a}, R^{3b}, R^{2c}, R^{2d}, R^{3c}, and R^{3d} are defined herein below in Table 13.

TABLE 13
Exemplary compounds of the formula (XXIV)

| Entry | R^{2a} | R^{2b} | R^{3a} | R^{3b} | R^{2c} | R^{2d} | R^{3c} | R^{3d} |
|---|---|---|---|---|---|---|---|---|
| 1 | H | H | H | H | H | H | H | H |
| 2 | H | F | H | H | H | H | H | H |
| 3 | F | H | H | H | H | H | H | H |
| 4 | H | H | H | H | F | H | H | H |
| 5 | H | H | H | H | H | F | H | H |
| 6 | H | Cl | H | H | H | H | H | H |
| 7 | Cl | H | H | H | H | H | H | H |
| 8 | H | H | H | H | Cl | H | H | H |
| 9 | H | H | H | H | H | Cl | H | H |
| 10 | H | OMe | H | H | H | H | H | H |
| 11 | OMe | H | H | H | H | H | H | H |
| 12 | H | H | H | H | OMe | H | H | H |
| 13 | H | H | H | H | H | OMe | H | h |
| 14 | H | CF₃ | H | H | H | H | H | H |
| 15 | CF₃ | H | H | H | H | H | H | H |
| 16 | H | H | H | H | CF₃ | H | H | H |
| 17 | H | H | H | H | H | CF₃ | H | H |

Compounds of the present invention include compounds having the formula (XXV) or a pharmaceutically acceptable salt form thereof:

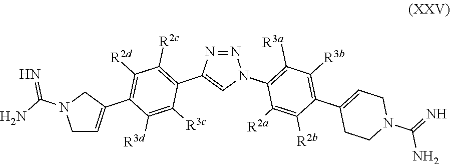

wherein non-limiting examples of R^{2a}, R^{2b}, R^{3a}, R^{3b}, R^{2c}, R^{2d}, R^{3c}, and R^{3d} are defined herein below in Table 14.

TABLE 14
Exemplary compounds of the formula (XXV)

| Entry | R^{2a} | R^{2b} | R^{3a} | R^{3b} | R^{2c} | R^{2d} | R^{3c} | R^{3d} |
|---|---|---|---|---|---|---|---|---|
| 1 | H | H | H | H | H | H | H | H |
| 2 | H | F | H | H | H | H | H | H |
| 3 | F | H | H | H | H | H | H | H |
| 4 | H | H | H | H | F | H | H | H |
| 5 | H | H | H | H | H | F | H | H |
| 6 | H | Cl | H | H | H | H | H | H |
| 7 | Cl | H | H | H | H | H | H | H |
| 8 | H | H | H | H | Cl | H | H | H |
| 9 | H | H | H | H | H | Cl | H | H |
| 10 | H | OMe | H | H | H | H | H | H |
| 11 | OMe | H | H | H | H | H | H | H |
| 12 | H | H | H | H | OMe | H | H | H |
| 13 | H | H | H | H | H | OMe | H | H |
| 14 | H | CF₃ | H | H | H | H | H | H |
| 15 | CF₃ | H | H | H | H | H | H | H |
| 16 | H | H | H | H | CF₃ | H | H | H |
| 17 | H | H | H | H | H | CF₃ | H | H |

Compounds of the present invention include compounds having the formula (XXVI) or a pharmaceutically acceptable salt form thereof:

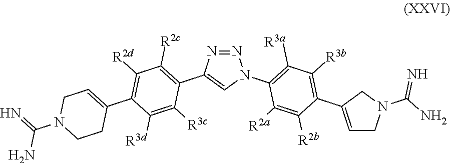

wherein non-limiting examples of R^{2a}, R^{2b}, R^{3a}, R^{3b}, R^{2c}, R^{2d}, R^{3c}, and R^{3d} are defined herein below in Table 15.

TABLE 15
Exemplary compounds of the formula (XXVI)

| Entry | R^{2a} | R^{2b} | R^{3a} | R^{3b} | R^{2c} | R^{2d} | R^{3c} | R^{3d} |
|---|---|---|---|---|---|---|---|---|
| 1 | H | H | H | H | H | H | H | H |
| 2 | H | F | H | H | H | H | H | H |
| 3 | F | H | H | H | H | H | H | H |
| 4 | H | H | H | H | F | H | H | H |
| 5 | H | H | H | H | H | F | H | H |
| 6 | H | Cl | H | H | H | H | H | H |
| 7 | Cl | H | H | H | H | H | H | H |
| 8 | H | H | H | H | Cl | H | H | H |
| 9 | H | H | H | H | H | Cl | H | H |
| 10 | H | OMe | H | H | H | H | H | H |
| 11 | OMe | H | H | H | H | H | H | H |
| 12 | H | H | H | H | OMe | H | H | H |
| 13 | H | H | H | H | H | OMe | H | H |
| 14 | H | CF₃ | H | H | H | H | H | H |
| 15 | CF₃ | H | H | H | H | H | H | H |
| 16 | H | H | H | H | CF₃ | H | H | H |
| 17 | H | H | H | H | H | CF₃ | H | H |

Compounds of the present invention include compounds having the formula (XXVII) or a pharmaceutically acceptable salt form thereof:

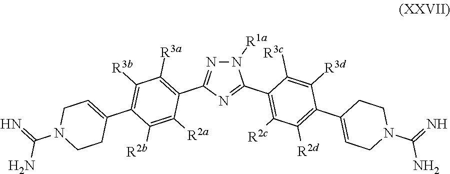

wherein non-limiting examples of R^{1a}, R^{2a}, R^{2b}, R^{3a}, R^{3b}, R^{2c}, R^{2d}, R^{3c}, and R^{3d} are defined herein below in Table 16.

TABLE 16
Exemplary compounds of the formula (XXVII)

| Entry | R^{1a} | R^{2a} | R^{2b} | R^{3a} | R^{3b} | R^{2c} | R^{2d} | R^{3c} | R^{3d} |
|---|---|---|---|---|---|---|---|---|---|
| 1a | Me | H | F | H | H | H | H | H | H |
| 1b | CH₂Me | H | F | H | H | H | H | H | H |
| 1c | CH₂CH₂OH | H | F | H | H | H | H | H | H |
| 1d | CH₂CH₂OMe | H | F | H | H | H | H | H | H |
| 2a | Me | F | H | H | H | H | H | H | H |
| 2b | CH₂Me | F | H | H | H | H | H | H | H |
| 2c | CH₂CH₂OH | F | H | H | H | H | H | H | H |
| 2d | CH₂CH₂OMe | F | H | H | H | H | H | H | H |
| 3a | Me | H | H | H | H | F | H | H | H |
| 3b | CH₂Me | H | H | H | H | F | H | H | H |
| 3c | CH₂CH₂OH | H | H | H | H | F | H | H | H |
| 3d | CH₂CH₂OMe | H | H | H | H | F | H | H | H |
| 4a | Me | H | H | H | H | H | F | H | H |
| 4b | CH₂Me | H | H | H | H | H | F | H | H |
| 4c | CH₂CH₂OH | H | H | H | H | H | F | H | H |
| 4d | CH₂CH₂OMe | H | H | H | H | H | F | H | H |
| 5a | Me | H | Cl | H | H | H | H | H | H |
| 5b | CH₂Me | H | Cl | H | H | H | H | H | H |
| 5c | CH₂CH₂OH | H | Cl | H | H | H | H | H | H |
| 5d | CH₂CH₂OMe | H | Cl | H | H | H | H | H | H |
| 6a | Me | Cl | H | H | H | H | H | H | H |
| 6b | CH₂Me | Cl | H | H | H | H | H | H | H |
| 6c | CH₂CH₂OH | Cl | H | H | H | H | H | H | H |
| 6d | CH₂CH₂OMe | Cl | H | H | H | H | H | H | H |
| 7a | Me | H | H | H | H | Cl | H | H | H |
| 7b | CH₂Me | H | H | H | H | Cl | H | H | H |
| 7c | CH₂CH₂OH | H | H | H | H | Cl | H | H | H |
| 7d | CH₂CH₂OMe | H | H | H | H | Cl | H | H | H |
| 8a | Me | H | H | H | H | H | Cl | H | H |
| 8b | CH₂Me | H | H | H | H | H | Cl | H | H |
| 8c | CH₂CH₂OH | H | H | H | H | H | Cl | H | H |
| 8d | CH₂CH₂OMe | H | H | H | H | H | Cl | H | H |
| 9a | Me | H | H | H | H | Me | H | H | H |
| 9b | CH₂Me | H | H | H | H | Me | H | H | H |
| 9c | CH₂CH₂OH | H | H | H | H | Me | H | H | H |
| 9d | CH₂CH₂OMe | H | H | H | H | Me | H | H | H |
| 10a | Me | H | H | H | H | H | Me | H | H |
| 10b | CH₂Me | H | H | H | H | H | Me | H | H |
| 10c | CH₂CH₂OH | H | H | H | H | H | Me | H | H |
| 10d | CH₂CH₂OMe | H | H | H | H | H | Me | H | H |
| 11a | Me | H | OMe | H | H | H | H | H | H |
| 11b | CH₂Me | H | OMe | H | H | H | H | H | H |
| 11c | CH₂CH₂OH | H | OMe | H | H | H | H | H | H |
| 11d | CH₂CH₂OMe | H | OMe | H | H | H | H | H | H |
| 12a | Me | OMe | H | H | H | H | H | H | H |
| 12b | CH₂Me | OMe | H | H | H | H | H | H | H |
| 12c | CH₂CH₂OH | OMe | H | H | H | H | H | H | H |
| 12d | CH₂CH₂OMe | OMe | H | H | H | H | H | H | H |
| 13a | Me | H | H | H | H | OMe | H | H | H |
| 13b | CH₂Me | H | H | H | H | OMe | H | H | H |
| 13c | CH₂CH₂OH | H | H | H | H | OMe | H | H | H |
| 13d | CH₂CH₂OMe | H | H | H | H | OMe | H | H | H |
| 14a | Me | H | H | H | H | H | OMe | H | H |
| 14b | CH₂Me | H | H | H | H | H | OMe | H | H |
| 14c | CH₂CH₂OH | H | H | H | H | H | OMe | H | H |
| 14d | CH₂CH₂OMe | H | H | H | H | H | OMe | H | H |
| 15a | Me | H | CF₃ | H | H | H | H | H | H |
| 15b | CH₂Me | H | CF₃ | H | H | H | H | H | H |
| 15c | CH₂CH₂OH | H | CF₃ | H | H | H | H | H | H |
| 15d | CH₂CH₂OMe | H | CF₃ | H | H | H | H | H | H |
| 16a | Me | CF₃ | H | H | H | H | H | H | H |
| 16b | CH₂Me | CF₃ | H | H | H | H | H | H | H |
| 16c | CH₂CH₂OH | CF₃ | H | H | H | H | H | H | H |
| 16d | CH₂CH₂OMe | CF₃ | H | H | H | H | H | H | H |
| 17a | Me | H | H | H | H | CF₃ | H | H | H |
| 17b | CH₂Me | H | H | H | H | CF₃ | H | H | H |
| 17c | CH₂CH₂OH | H | H | H | H | CF₃ | H | H | H |
| 17d | CH₂CH₂OMe | H | H | H | H | CF₃ | H | H | H |
| 18a | Me | H | H | H | H | H | CF₃ | H | H |
| 18b | CH₂Me | H | H | H | H | H | CF₃ | H | H |
| 18c | CH₂CH₂OH | H | H | H | H | H | CF₃ | H | H |
| 18d | CH₂CH₂OMe | H | H | H | H | H | CF₃ | H | H |
| 19 | Ethyl | H | H | H | H | H | H | H | H |
| 20 | Ethyl | H | Me | H | H | H | H | H | H |
| 21 | Ethyl | Me | H | H | H | H | H | H | H |
| 22 | Ethyl | OMe | H | H | H | H | H | H | H |

Compounds of the present invention include compounds having the formula (XXVII) or a pharmaceutically acceptable salt form thereof:

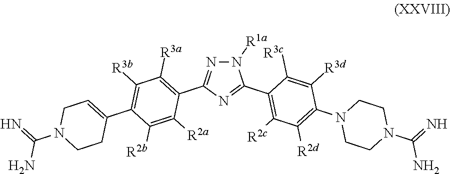

wherein non-limiting examples of R^{1a}, R^{2a}, R^{2b}, R^{3a}, R^{3b}, R^{2c}, R^{2d}, R^{3c}, and R^{3d} are defined herein below in Table 17.

TABLE 17
Exemplary compounds of the formula (XXVIII)

| Entry | R^{1a} | R^{2a} | R^{2b} | R^{3a} | R^{3b} | R^{2c} | R^{2d} | R^{3c} | R^{3d} |
|---|---|---|---|---|---|---|---|---|---|
| 1a | Me | H | H | H | H | H | H | H | H |
| 1b | CH₂Me | H | H | H | H | H | H | H | H |
| 1c | CH₂CH₂OH | H | H | H | H | H | H | H | H |
| 1d | CH₂CH₂OMe | H | H | H | H | H | H | H | H |
| 2a | Me | H | F | H | H | H | H | H | H |
| 2b | CH₂Me | H | F | H | H | H | H | H | H |
| 2c | CH₂CH₂OH | H | F | H | H | H | H | H | H |
| 2d | CH₂CH₂OMe | H | F | H | H | H | H | H | H |
| 3a | Me | F | H | H | H | H | H | H | H |
| 3b | CH₂Me | F | H | H | H | H | H | H | H |
| 3c | CH₂CH₂OH | F | H | H | H | H | H | H | H |
| 3d | CH₂CH₂OMe | F | H | H | H | H | H | H | H |
| 4a | Me | H | H | H | H | F | H | H | H |
| 4b | CH₂Me | H | H | H | H | F | H | H | H |
| 4c | CH₂CH₂OH | H | H | H | H | F | H | H | H |
| 4d | CH₂CH₂OMe | H | H | H | H | F | H | H | H |
| 5a | Me | H | H | H | H | H | F | H | H |
| 5b | CH₂Me | H | H | H | H | H | F | H | H |
| 5c | CH₂CH₂OH | H | H | H | H | H | F | H | H |
| 5d | CH₂CH₂OMe | H | H | H | H | H | F | H | H |
| 6a | Me | H | Cl | H | H | H | H | H | H |
| 6b | CH₂Me | H | Cl | H | H | H | H | H | H |
| 6c | CH₂CH₂OH | H | Cl | H | H | H | H | H | H |
| 6d | CH₂CH₂OMe | H | Cl | H | H | H | H | H | H |
| 7a | Me | Cl | H | H | H | H | H | H | H |
| 7b | CH₂Me | Cl | H | H | H | H | H | H | H |
| 7c | CH₂CH₂OH | Cl | H | H | H | H | H | H | H |
| 7d | CH₂CH₂OMe | Cl | H | H | H | H | H | H | H |
| 8a | Me | H | H | H | H | Cl | H | H | H |
| 8b | CH₂Me | H | H | H | H | Cl | H | H | H |
| 8c | CH₂CH₂OH | H | H | H | H | Cl | H | H | H |
| 8d | CH₂CH₂OMe | H | H | H | H | Cl | H | H | H |
| 9a | Me | H | H | H | H | H | Cl | H | H |
| 9b | CH₂Me | H | H | H | H | H | Cl | H | H |
| 9c | CH₂CH₂OH | H | H | H | H | H | Cl | H | H |
| 9d | CH₂CH₂OMe | H | H | H | H | H | Cl | H | H |
| 10a | Me | H | H | H | H | Me | H | H | H |
| 10b | CH₂Me | H | H | H | H | Me | H | H | H |
| 10c | CH₂CH₂OH | H | H | H | H | Me | H | H | H |
| 10d | CH₂CH₂OMe | H | H | H | H | Me | H | H | H |
| 11a | Me | H | H | H | H | H | Me | H | H |
| 11b | CH₂Me | H | H | H | H | H | Me | H | H |
| 11c | CH₂CH₂OH | H | H | H | H | H | Me | H | H |
| 11d | CH₂CH₂OMe | H | H | H | H | H | Me | H | H |
| 12a | Me | H | OMe | H | H | H | H | H | H |
| 12b | CH₂Me | H | OMe | H | H | H | H | H | H |
| 12c | CH₂CH₂OH | H | OMe | H | H | H | H | H | H |
| 12d | CH₂CH₂OMe | H | OMe | H | H | H | H | H | H |
| 13a | Me | OMe | H | H | H | H | H | H | H |
| 13b | CH₂Me | OMe | H | H | H | H | H | H | H |
| 13c | CH₂CH₂OH | OMe | H | H | H | H | H | H | H |
| 13d | CH₂CH₂OMe | OMe | H | H | H | H | H | H | H |
| 14a | Me | H | H | H | H | OMe | H | H | H |
| 14b | CH₂Me | H | H | H | H | OMe | H | H | H |
| 14c | CH₂CH₂OH | H | H | H | H | OMe | H | H | H |
| 14d | CH₂CH₂OMe | H | H | H | H | OMe | H | H | H |
| 15a | Me | H | H | H | H | H | OMe | H | H |
| 15b | CH₂Me | H | H | H | H | H | OMe | H | H |
| 15c | CH₂CH₂OH | H | H | H | H | H | OMe | H | H |
| 15d | CH₂CH₂OMe | H | H | H | H | H | OMe | H | H |
| 16a | Me | H | CF₃ | H | H | H | H | H | H |
| 16b | CH₂Me | H | CF₃ | H | H | H | H | H | H |
| 16c | CH₂CH₂OH | H | CF₃ | H | H | H | H | H | H |
| 16d | CH₂CH₂OMe | H | CF₃ | H | H | H | H | H | H |
| 17a | Me | CF₃ | H | H | H | H | H | H | H |
| 17b | CH₂Me | CF₃ | H | H | H | H | H | H | H |
| 17c | CH₂CH₂OH | CF₃ | H | H | H | H | H | H | H |
| 17d | CH₂CH₂OMe | CF₃ | H | H | H | H | H | H | H |
| 18a | Me | H | H | H | H | CF₃ | H | H | H |
| 18b | CH₂Me | H | H | H | H | CF₃ | H | H | H |
| 18c | CH₂CH₂OH | H | H | H | H | CF₃ | H | H | H |
| 18d | CH₂CH₂OMe | H | H | H | H | CF₃ | H | H | H |
| 19a | Me | H | H | H | H | H | CF₃ | H | H |
| 19b | CH₂Me | H | H | H | H | H | CF₃ | H | H |
| 19c | CH₂CH₂OH | H | H | H | H | H | CF₃ | H | H |
| 19d | CH₂CH₂OMe | H | H | H | H | H | CF₃ | H | H |
| 20 | Ethyl | H | Me | H | H | H | H | H | H |
| 21 | Ethyl | Me | H | H | H | H | H | H | H |

Compounds of the present invention include compounds having the formula (XXIX) or a pharmaceutically acceptable salt form thereof:

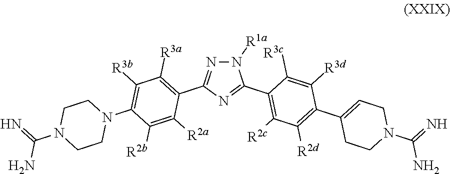

wherein non-limiting examples of R^{1a}, R^{2a}, R^{2b}, R^{3a}, R^{3b}, R^{2c}, R^{2d}, R^{3c}, and R^{3d} are defined herein below in Table 18.

TABLE 18
Exemplary compounds of the formula (XXIX)

| Entry | R^{1a} | R^{2a} | R^{2b} | R^{3a} | R^{3b} | R^{2c} | R^{2d} | R^{3c} | R^{3d} |
|---|---|---|---|---|---|---|---|---|---|
| 1a | Me | H | H | H | H | H | H | H | H |
| 1b | CH₂Me | H | H | H | H | H | H | H | H |
| 1c | CH₂CH₂OH | H | H | H | H | H | H | H | H |
| 1d | CH₂CH₂OMe | H | H | H | H | H | H | H | H |
| 2a | Me | H | F | H | H | H | H | H | H |
| 2b | CH₂Me | H | F | H | H | H | H | H | H |
| 2c | CH₂CH₂OH | H | F | H | H | H | H | H | H |
| 2d | CH₂CH₂OMe | H | F | H | H | H | H | H | H |
| 3a | Me | F | H | H | H | H | H | H | H |
| 3b | CH₂Me | F | H | H | H | H | H | H | H |
| 3c | CH₂CH₂OH | F | H | H | H | H | H | H | H |
| 3d | CH₂CH₂OMe | F | H | H | H | H | H | H | H |
| 4a | Me | H | H | H | H | F | H | H | H |
| 4b | CH₂Me | H | H | H | H | F | H | H | H |
| 4c | CH₂CH₂OH | H | H | H | H | F | H | H | H |
| 4d | CH₂CH₂OMe | H | H | H | H | F | H | H | H |
| 5a | Me | H | H | H | H | H | F | H | H |
| 5b | CH₂Me | H | H | H | H | H | F | H | H |
| 5c | CH₂CH₂OH | H | H | H | H | H | F | H | H |
| 5d | CH₂CH₂OMe | H | H | H | H | H | F | H | H |
| 6a | Me | H | Cl | H | H | H | H | H | H |
| 6b | CH₂Me | H | Cl | H | H | H | H | H | H |
| 6c | CH₂CH₂OH | H | Cl | H | H | H | H | H | H |
| 6d | CH₂CH₂OMe | H | Cl | H | H | H | H | H | H |
| 7a | Me | Cl | H | H | H | H | H | H | H |
| 7b | CH₂Me | Cl | H | H | H | H | H | H | H |
| 7c | CH₂CH₂OH | Cl | H | H | H | H | H | H | H |
| 7d | CH₂CH₂OMe | Cl | H | H | H | H | H | H | H |
| 8a | Me | H | H | H | H | Cl | H | H | H |
| 8b | CH₂Me | H | H | H | H | Cl | H | H | H |
| 8c | CH₂CH₂OH | H | H | H | H | Cl | H | H | H |
| 8d | CH₂CH₂OMe | H | H | H | H | Cl | H | H | H |
| 9a | Me | H | H | H | H | H | Cl | H | H |
| 9b | CH₂Me | H | H | H | H | H | Cl | H | H |
| 9c | CH₂CH₂OH | H | H | H | H | H | Cl | H | H |
| 9d | CH₂CH₂OMe | H | H | H | H | H | Cl | H | H |
| 10a | Me | H | H | H | H | Me | H | H | H |
| 10b | CH₂Me | H | H | H | H | Me | H | H | H |
| 10c | CH₂CH₂OH | H | H | H | H | Me | H | H | H |
| 10d | CH₂CH₂OMe | H | H | H | H | Me | H | H | H |
| 11a | Me | H | H | H | H | H | Me | H | H |
| 11b | CH₂Me | H | H | H | H | H | Me | H | H |
| 11c | CH₂CH₂OH | H | H | H | H | H | Me | H | H |
| 11d | CH₂CH₂OMe | H | H | H | H | H | Me | H | H |
| 12a | Me | H | OMe | H | H | H | H | H | H |
| 12b | CH₂Me | H | OMe | H | H | H | H | H | H |
| 12c | CH₂CH₂OH | H | OMe | H | H | H | H | H | H |
| 12d | CH₂CH₂OMe | H | OMe | H | H | H | H | H | H |
| 13a | Me | OMe | H | H | H | H | H | H | H |
| 13b | CH₂Me | OMe | H | H | H | H | H | H | H |
| 13c | CH₂CH₂OH | OMe | H | H | H | H | H | H | H |
| 13d | CH₂CH₂OMe | OMe | H | H | H | H | H | H | H |
| 14a | Me | H | H | H | H | OMe | H | H | H |
| 14b | CH₂Me | H | H | H | H | OMe | H | H | H |
| 14c | CH₂CH₂OH | H | H | H | H | OMe | H | H | H |
| 14d | CH₂CH₂OMe | H | H | H | H | OMe | H | H | H |
| 15a | Me | H | H | H | H | H | OMe | H | H |
| 15b | CH₂Me | H | H | H | H | H | OMe | H | H |
| 15c | CH₂CH₂OH | H | H | H | H | H | OMe | H | H |
| 15d | CH₂CH₂OMe | H | H | H | H | H | OMe | H | H |
| 16a | Me | H | CF₃ | H | H | H | H | H | H |
| 16b | CH₂Me | H | CF₃ | H | H | H | H | H | H |
| 160 | CH₂CH₂OH | H | CF₃ | H | H | H | H | H | H |
| 16d | CH₂CH₂OMe | H | CF₃ | H | H | H | H | H | H |
| 17a | Me | CF₃ | H | H | H | H | H | H | H |
| 17b | CH₂Me | CF₃ | H | H | H | H | H | H | H |
| 17c | CH₂CH₂OH | CF₃ | H | H | H | H | H | H | H |
| 17d | CH₂CH₂OMe | CF₃ | H | H | H | H | H | H | H |
| 18a | Me | H | H | H | H | CF₃ | H | H | H |
| 18b | CH₂Me | H | H | H | H | CF₃ | H | H | H |
| 18c | CH₂CH₂OH | H | H | H | H | CF₃ | H | H | H |
| 18d | CH₂CH₂OMe | H | H | H | H | CF₃ | H | H | H |
| 19a | Me | H | H | H | H | H | CF₃ | H | H |
| 19b | CH₂Me | H | H | H. | H | H | CF₃ | H | H |
| 19c | CH₂CH₂OH | H | H | H | H | H | CF₃ | H | H |
| 19d | CH₂CH₂OMe | H | H | H | H | H | CF₃ | H | H |
| 20 | Ethyl | H | Me | H | H | H | H | H | H |
| 21 | Ethyl | Me | H | H | H | H | H | H | H |

Compounds of the present invention include compounds having the formula (XXX) or a pharmaceutically acceptable salt form thereof:

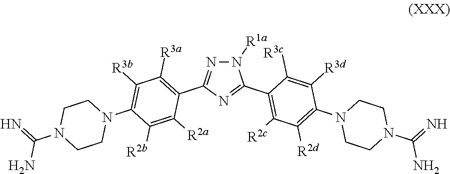

wherein non-limiting examples of R^{1a}, R^{2a}, R^{2b}, R^{3a}, R^{3b}, R^{2c}, R^{2d}, R^{3c}, and R^{3d} are defined herein below in Table 19.

TABLE 19
Exemplary compounds of the formula (XXX)

| Entry | R^{1a} | R^{2a} | R^{2b} | R^{3a} | R^{3b} | R^{2c} | R^{2d} | R^{3c} | R^{3d} |
|---|---|---|---|---|---|---|---|---|---|
| 1a | Me | H | H | H | H | H | H | H | H |
| 1b | CH₂Me | H | H | H | H | H | H | H | H |
| 1c | CH₂CH₂OH | H | H | H | H | H | H | H | H |
| 1d | CH₂CH₂OMe | H | H | H | H | H | H | H | H |
| 2a | Me | H | F | H | H | H | H | H | H |
| 2b | CH₂Me | H | F | H | H | H | H | H | H |
| 2c | CH₂CH₂OH | H | F | H | H | H | H | H | H |
| 2d | CH₂CH₂OMe | H | F | H | H | H | H | H | H |
| 3a | Me | F | H | H | H | H | H | H | H |
| 3b | CH₂Me | F | H | H | H | H | H | H | H |
| 3c | CH₂CH₂OH | F | H | H | H | H | H | H | H |
| 3d | CH₂CH₂OMe | F | H | H | H | H | H | H | H |
| 4a | Me | H | H | H | H | F | H | H | H |
| 4b | CH₂Me | H | H | H | H | F | H | H | H |
| 4c | CH₂CH₂OH | H | H | H | H | F | H | H | H |
| 4d | CH₂CH₂OMe | H | H | H | H | F | H | H | H |
| 5a | Me | H | H | H | H | H | F | H | H |
| 5b | CH₂Me | H | H | H | H | H | F | H | H |
| 5c | CH₂CH₂OH | H | H | H | H | H | F | H | H |
| 5d | CH₂CH₂OMe | H | H | H | H | H | F | H | H |
| 6a | Me | H | Cl | H | H | H | H | H | H |
| 6b | CH₂Me | H | Cl | H | H | H | H | H | H |
| 6c | CH₂CH₂OH | H | Cl | H | H | H | H | H | H |
| 6d | CH₂CH₂OMe | H | Cl | H | H | H | H | H | H |
| 7a | Me | Cl | H | H | H | H | H | H | H |
| 7b | CH₂Me | Cl | H | H | H | H | H | H | H |
| 7c | CH₂CH₂OH | Cl | H | H | H | H | H | H | H |
| 7d | CH₂CH₂OMe | Cl | H | H | H | H | H | H | H |
| 8a | Me | H | H | H | H | Cl | H | H | H |
| 8b | CH₂Me | H | H | H | H | Cl | H | H | H |
| 8c | CH₂CH₂OH | H | H | H | H | Cl | H | H | H |
| 8d | CH₂CH₂OMe | H | H | H | H | Cl | H | H | H |
| 9a | Me | H | H | H | H | H | Cl | H | H |
| 9b | CH₂Me | H | H | H | H | H | Cl | H | H |
| 9c | CH₂CH₂OH | H | H | H | H | H | Cl | H | H |
| 9d | CH₂CH₂OMe | H | H | H | \|H | H | Cl | H | H |
| 10a | Me | H | H | H | H | Me | H | H | H |
| 10b | CH₂Me | H | H | H | H | Me | H | H | H |
| 10c | CH₂CH₂OH | H | H | H | H | Me | H | H | H |
| 10d | CH₂CH₂OMe | H | H | H | H | Me | H | H | H |
| 11a | Me | H | H | H | H | H | Me | H | H |
| 11b | CH₂Me | H | H | H | H | H | Me | H | H |
| 11c | CH₂CH₂OH | H | H | H | H | H | Me | H | H |
| 11d | CH₂CH₂OMe | H | H | H | H | H | Me | H | H |
| 12a | Me | H | OMe | H | H | H | H | H | H |
| 12b | CH₂Me | H | OMe | H | H | H | H | H | H |
| 12c | CH₂CH₂OH | H | OMe | H | H | H | H | H | H |
| 12d | CH₂CH₂OMe | H | OMe | H | H | H | H | H | H |
| 13a | Me | OMe | H | H | H | H | H | H | H |
| 13b | CH₂Me | OMe | H | H | H | H | H | H | H |
| 13c | CH₂CH₂OH | OMe | H | H | H | H | H | H | H |
| 13d | CH₂CH₂OMe | OMe | H | H | H | H | H | H | H |
| 14a | Me | H | H | H | H | OMe | H | H | H |
| 14b | CH₂Me | H | H | H | H | OMe | H | H | H |
| 14c | CH₂CH₂OH | H | H | H | H | OMe | H | H | H |
| 14d | CH₂CH₂OMe | H | H | H | H | OMe | H | H | H |
| 15a | Me | H | H | H | H | H | OMe | H | H |
| 15b | CH₂Me | H | H | H | H | H | OMe | H | H |
| 15c | CH₂CH₂OH | H | H | H | H | H | OMe | H | H |
| 15d | CH₂CH₂OMe | H | H | H | H | H | OMe | H | H |
| 16a | Me | H | CF₃ | H | H | H | H | H | H |
| 16b | CH₂Me | H | CF₃ | H | H | H | H | H | H |
| 16c | CH₂CH₂OH | H | CF₃ | H | H | H | H | H | H |
| 16d | CH₂CH₂OMe | H | CF₃ | H | H | H | H | H | H |
| 17a | Me | CF₃ | H | H | H | H | H | H | H |
| 17b | CH₂Me | CF₃ | H | H | H | H | H | H | H |
| 17c | CH₂CH₂OH | CF₃ | H | H | H | H | H | H | H |
| 17d | CH₂CH₂OMe | CF₃ | H | H | H | H | H | H | H |
| 18a | Me | H | H | H | H | CF₃ | H | H | H |
| 18b | CH₂Me | H | H | H | H | CF₃ | H | H | H |
| 18c | CH₂CH₂OH | H | H | H | H | CF₃ | H | H | H |
| 18d | CH₂CH₂OMe | H | H | H. | H | CF₃ | H | H | H |
| 19a | Me | H | H | H | H | H | CF₃ | H | H |
| 19b | CH₂Me | H | H | H | H | H | CF₃ | H | H |
| 19c | CH₂CH₂OH | H | H | H | H | H | CF₃ | H | H |
| 19d | CH₂CH₂OMe | H | H | H | H | H | CF₃ | H | H |
| 20 | Ethyl | H | Me | H | H | H | H | H | H |
| 21 | Ethyl | Me | H | H | H | H | H | H | H |

Compounds of the present invention include compounds having the formula (XXXI) or a pharmaceutically acceptable salt form thereof:

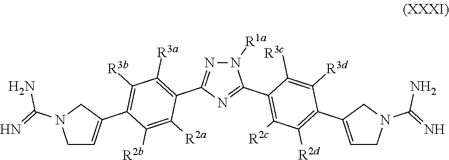

wherein non-limiting examples of R^{1a}, R^{2a}, R^{2b}, R^{3a}, R^{3b}, R^{2c}, R^{2d}, R^{3c}, and R^{3d} are defined herein below in Table 20.

TABLE 20
Exemplary compounds of the formula (XXXI)

| Entry | R^{1a} | R^{2a} | R^{2b} | R^{3a} | R^{3b} | R^{2c} | R^{2d} | R^{3c} | R^{3d} |
|---|---|---|---|---|---|---|---|---|---|
| 1a | Me | H | H | H | H | H | H | H | H |
| 1b | CH₂Me | H | H | H | H | H | H | H | H |
| 1c | CH₂CH₂OH | H | H | H | H | H | H | H | H |
| 1d | CH₂CH₂OMe | H | H | H | H | H | H | H | H |
| 2a | Me | H | F | H | H | H | H | H | H |
| 2b | CH₂Me | H | F | H | H | H | H | H | H |
| 2c | CH₂CH₂OH | H | F | H | H | H | H | H | H |
| 2d | CH₂CH₂OMe | H | F | H | H | H | H | H | H |
| 3a | Me | F | H | H | H | H | H | H | H |
| 3b | CH₂Me | F | H | H | H | H | H | H | H |
| 3c | CH₂CH₂OH | F | H | H | H | H | H | H | H |
| 3d | CH₂CH₂OMe | F | H | H | H | H | H | H | H |
| 4a | Me | H | H | H | H | F | H | H | H |
| 4b | CH₂Me | H | H | H | H | F | H | H | H |
| 4c | CH₂CH₂OH | H | H | H | H | F | H | H | H |
| 4d | CH₂CH₂OMe | H | H | H | H | F | H | H | H |
| 5 | Me | H | H | H | H | H | F | H | H |
| 5b | CH₂Me | H | H | H | H | H | F | H | H |
| 5c | CH₂CH₂OH | H | H | H | H | H | F | H | H |
| 5d | CH₂CH₂OMe | H | H | H | H | H | F | H | H |
| 6a | Me | H | Cl | H | H | H | H | H | H |
| 6b | CH₂Me | H | Cl | H | H | H | H | H | H |
| 6c | CH₂CH₂OH | H | Cl | H | H | H | H | H | H |
| 6d | CH₂CH₂OMe | H | Cl | H | H | H | H | H | H |
| 7a | Me | Cl | H | H | H | H | H | H | H |
| 7b | CH₂Me | Cl | H | H | H | H | H | H | H |
| 7c | CH₂CH₂OH | Cl | H | H | H | H | H | H | H |
| 7d | CH₂CH₂OMe | Cl | H | H | H | H | H | H | H |
| 8a | Me | H | H | H | H | Cl | H | H | H |
| 8b | CH₂Me | H | H | H | H | Cl | H | H | H |
| 8c | CH₂CH₂OH | H | H | H | H | Cl | H | H | H |
| 8d | CH₂CH₂OMe | H | H | H | H | Cl | H | H | H |
| 9a | Me | H | H | H | H | H | Cl | H | H |
| 9b | CH₂Me | H | H | H | H | H | Cl | H | H |
| 9c | CH₂CH₂OH | H | H | H | H | H | Cl | H | H |
| 9d | CH₂CH₂OMe | H | H | H | H | H | Cl | H | H |
| 10a | Me | H | H | H | H | Me | H | H | H |
| 10b | CH₂Me | H | H | H | H | Me | H | H | H |
| 10c | CH₂CH₂OH | H | H | H | H | Me | H | H | H |
| 10d | CH₂CH₂OMe | H | H | H | H | Me | H | H | H |
| 11a | Me | H | H | H | H | H | Me | H | H |
| 11b | CH₂Me | H | H | H | H | H | Me | H | H |
| 11c | CH₂CH₂OH | H | H | H | H | H | Me | H | H |
| 11d | CH₂CH₂OMe | H | H | H | H | H | Me | H | H |
| 12a | Me | H | OMe | H | H | H | H | H | H |
| 12b | CH₂Me | H | OMe | H | H | H | H | H | H |
| 12c | CH₂CH₂OH | H | OMe | H | H | H | H | H | H |
| 12d | CH₂CH₂OMe | H | OMe | H | H | H | H | H | H |
| 13a | Me | OMe | H | H | H | H | H | H | H |
| 13b | CH₂Me | OMe | H | H | H | H | H | H | H |
| 13c | CH₂CH₂OH | OMe | H | H | H | H | H | H | H |
| 13d | CH₂CH₂OMe | OMe | H | H | H | H | H | H | H |
| 14a | Me | H | H | H | H | OMe | H | H | H |
| 14b | CH₂Me | H | H | H | H | OMe | H | H | H |
| 14c | CH₂CH₂OH | H | H | H | H | OMe | H | H | H |
| 14d | CH₂CH₂OMe | H | H | H | H | OMe | H | H | H |
| 15a | Me | H | H | H | H | H | OMe | H | H |
| 15b | CH₂Me | H | H | H | H | H | OMe | H | H |
| 15c | CH₂CH₂OH | H | H | H | H | H | OMe | H | H |
| 15d | CH₂CH₂OMe | H | H | H | H | H | OMe | H | H |
| 16a | Me | H | CF₃ | H | H | H | H | H | H |
| 16b | CH₂Me | H | CF₃ | H | H | H | H | H | H |
| 16c | CH₂CH₂OH | H | CF₃ | H | H | H | H | H | H |
| 16d | CH₂CH₂OMe | H | CF₃ | H | H | H | H | H | H |
| 17a | Me | CF₃ | H | H | H | H | H | H | H |
| 17b | CH₂Me | CF₃ | H | H | H | H | H | H | H |
| 17c | CH₂CH₂OH | CF₃ | H | H | H | H | H | H | H |
| 17d | CH₂CH₂OMe | CF₃ | H | H | H | H | H | H | H |
| 18a | Me | H | H | H | H | CF₃ | H | H | H |
| 18b | CH₂Me | H | H | H | H | CF₃ | H | H | H |
| 18c | CH₂CH₂OH | H | H | H | H | CF₃ | H | H | H |
| 18d | CH₂CH₂OMe | H | H | H | H | CF₃ | H | H | H |
| 19a | Me | H | H | H | H | H | CF3 | H | H |
| 19b | CH₂Me | H | H | H | H | H | CF3 | H | H |
| 19c | CH₂CH₂OH | H | H | H | H | H | CF3 | H | H |
| 19d | CH₂CH₂OMe | H | H | H | H | H | CF3 | H | H |
| 20 | Ethyl | H | Me | H | H | H | H | H | H |
| 21 | Ethyl | Me | H | H | H | H | H | H | H |

Compounds of the present invention include compounds having the formula (XXXII) or a pharmaceutically acceptable salt form thereof:

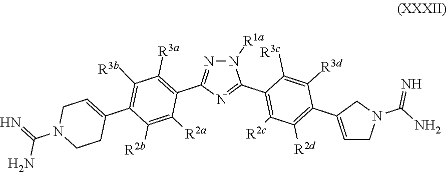

wherein non-limiting examples of R^{1a}, R^{2a}, R^{2b}, R^{3a}, R^{3b}, R^{2c}, R^{2d}, R^{3c}, and R^{3d} are defined herein below in Table 21.

TABLE 21
Exemplary compounds of the formula (XXXII)

| Entry | R^{1a} | R^{2a} | R^{2b} | R^{3a} | R^{3b} | R^{2c} | R^{2d} | R^{3c} | R^{3d} |
|---|---|---|---|---|---|---|---|---|---|
| 1a | Me | H | H | H | H | H | H | H | H |
| 1b | CH₂Me | H | H | H | H | H | H | H | H |
| 1c | CH₂CH₂OH | H | H | H | H | H | H | H | H |
| 1d | CH₂CH₂OMe | H | H | H | H | H | H | H | H |
| 2a | Me | H | F | H | H | H | H | H | H |
| 2b | CH₂Me | H | F | H | H | H | H | H | H |
| 2c | CH₂CH₂OH | H | F | H | H | H | H | H | H |
| 2d | CH₂CH₂OMe | H | F | H | H | H | H | H | H |
| 3a | Me | F | H | H | H | H | H | H | H |
| 3b | CH₂Me | F | H | H | H | H | H | H | H |
| 3c | CH₂CH₂OH | F | H | H | H | H | H | H | H |
| 3d | CH₂CH₂OMe | F | H | H | H | H | H | H | H |
| 4a | Me | H | H | H | H | F | H | H | H |
| 4b | CH₂Me | H | H | H | H | F | H | H | H |
| 4c | CH₂CH₂OH | H | H | H | H | F | H | H | H |
| 4d | CH₂CH₂OMe | H | H | H | H | F | H | H | H |
| 5a | Me | H | H | H | H | H | F | H | H |
| 5b | CH₂Me | H | H | H | H | H | F | H | H |
| 5c | CH₂CH₂OH | H | H | H | H | H | F | H | H |
| 5d | CH₂CH₂OMe | H | H | H | H | H | F | H | H |
| 6a | Me | H | Cl | H | H | H | H | H | H |
| 6b | CH₂Me | H | Cl | H | H | H | H | H | H |
| 6c | CH₂CH₂OH | H | Cl | H | H | H | H | H | H |
| 6d | CH₂CH₂OMe | H | Cl | H | H | H | H | H | H |
| 7a | Me | Cl | H | H | H | H | H | H | H |
| 7b | CH₂Me | Cl | H | H | H | H | H | H | H |
| 7c | CH₂CH₂OH | Cl | H | H | H | H | H | H | H |
| 7d | CH₂CH₂OMe | Cl | H | H | H | H | H | H | H |
| 8a | Me | H | H | H | H | Cl | H | H | H |
| 8b | CH₂Me | H | H | H | H | Cl | H | H | H |
| 8c | CH₂CH₂OH | H | H | H | H | Cl | H | H | H |
| 8d | CH₂CH₂OMe | H | H | H | H | Cl | H | H | H |
| 9a | Me | H | H | H | H | H | Cl | H | H |
| 9b | CH₂Me | H | H | H | H | H | Cl | H | H |
| 9c | CH₂CH₂OH | H | H | H | H | H | Cl | H | H |
| 9d | CH₂CH₂OMe | H | H | H | H | H | Cl | H | H |
| 10a | Me | H | H | H | H | Me | H | H | H |
| 10b | CH₂Me | H | H | H | H | Me | H | H | H |
| 10c | CH₂CH₂OH | H | H | H | H | Me | H | H | H |
| 10d | CH₂CH₂OMe | H | H | H | H | Me | H | H | H |
| 11a | Me | H | H | H | H | H | Me | H | H |
| 11b | CH₂Me | H | H | H | H | H | Me | H | H |
| 11c | CH₂CH₂OH | H | H | H | H | H | Me | H | H |
| 11d | CH₂CH₂OMe | H | H | H | H | H | Me | H | H |
| 12a | Me | H | OMe | H | H | H | H | H | H |
| 12b | CH₂Me | H | OMe | H | H | H | H | H | H |
| 12c | CH₂CH₂OH | H | OMe | H | H | H | H | H | H |
| 12d | CH₂CH₂OMe | H | OMe | H | H | H | H | H | H |
| 13a | Me | OMe | H | H | H | H | H | H | H |
| 13b | CH₂Me | OMe | H | H | H | H | H | H | H |
| 13c | CH₂CH₂OH | OMe | H | H | H | H | H | H | H |
| 13d | CH₂CH₂OMe | OMe | H | H | H | H | H | H | H |
| 14a | Me | H | H | H | H | OMe | H | H | H |
| 14b | CH₂Me | H | H | H | H | OMe | H | H | H |
| 14c | CH₂CH₂OH | H | H | H | H | OMe | H | H | H |
| 14d | CH₂CH₂OMe | H | H | H | H | OMe | H | H | H |
| 15a | Me | H | H | H | H | H | OMe | H | H |
| 15b | CH₂Me | H | H | H | H | H | OMe | H | H |
| 15c | CH₂CH₂OH | H | H | H. | H | H | OMe | H | H |
| 15d | CH₂CH₂OMe | H | H | H | H | H | OMe | H | H |
| 16a | Me | H | CF₃ | H | H | H | H | H | H |
| 16b | CH₂Me | H | CF₃ | H | H | H | H | H | H |
| 16c | CH₂CH₂OH | H | CF₃ | H | H | H | H | H | H |
| 16d | CH₂CH₂OMe | H | CF₃ | H | H | H | H | H | H |
| 17a | Me | CF₃ | H | H | H | H | H | H | H |
| 17b | CH₂Me | CF₃ | H | H | H | H | H | H | H |
| 17c | CH₂CH₂OH | CF₃ | H | H | H | H | H | H | H |
| 17d | CH₂CH₂OMe | CF₃ | H | H | H | H | H | H | H |
| 18a | Me | H | H | H | H | CF₃ | H | H | H |
| 18b | CH₂Me | H | H | H | H | CF₃ | H | H | H |
| 18c | CH₂CH₂OH | H | H | H | H | CF₃ | H | H | H |
| 18d | CH₂CH₂OMe | H | H | H | H | CF₃ | H | H | H |
| 19a | Me | H | H | H | H | H | CF₃ | H | H |
| 19b | CH₂Me | H | H | H | H | H | CF₃ | H | H |
| 19c | CH₂CH₂OH | H | H | H | H | H | CF₃ | H | H |
| 19d | CH₂CH₂OMe | H | H | H | H | H | CF₃ | H | H |
| 20 | Ethyl | H | Me | H | H | H | H | H | H |
| 21 | Ethyl | Me | H | H | H | H | H | H | H |

Compounds of the present invention include compounds having the formula (XXXIII) or a pharmaceutically acceptable salt form thereof:

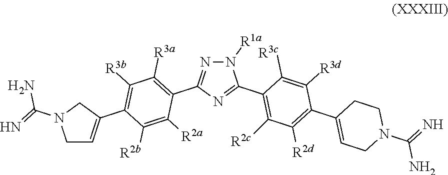

wherein non-limiting examples of R^{1a}, R^{2a}, R^{2b}, R^{3a}, R^{3b}, R^{2c}, R^{2d}, R^{3c}, and R^{3d} are defined herein below in Table 22.

TABLE 22
Exemplary compounds of the formula (XXXIII)

| Entry | R^{1a} | R^{2a} | R^{2b} | R^{3a} | R^{3b} | R^{2c} | R^{2d} | R^{3c} | R^{3d} |
|---|---|---|---|---|---|---|---|---|---|
| 1a | Me | H | H | H | H | H | H | H | H |
| 1b | CH₂Me | H | H | H | H | H | H | H | H |
| 1c | CH₂CHOH | H | H | H | H | H | H | H | H |
| 1d | CH₂CH₂OMe | H | H | H | H | H | H | H | H |
| 2a | Me | H | F | H | H | H | H | H | H |
| 2b | CH₂Me | H | F | H | H | H | H | H | H |
| 2c | CH₂CHOH | H | F | H | H | H | H | H | H |
| 2d | CH₂CH₂OMe | H | F | H | H | H | H | H | H |
| 3a | Me | F | H | H | H | H | H | H | H |
| 3b | CH₂Me | F | H | H | H | H | H | H | H |
| 3c | CH₂CHOH | F | H | H | H | H | H | H | H |
| 3d | CH₂CH₂OMe | F | H | H | H | H | H | H | H |
| 4a | Me | H | H | H | H | F | H | H | H |
| 4b | CH₂Me | H | H | H | H | F | H | H | H |
| 4c | CH₂CHOH | H | H | H | H | F | H | H | H |
| 4d | CH₂CH₂OMe | H | H | H | H | F | H | H | H |
| 5a | Me | H | H | H | H | H | F | H | H |
| 5b | CH₂Me | H | H | H | H | H | F | H | H |
| 5c | CH₂CHOH | H | H | H | H | H | F | H | H |
| 5d | CH₂CH₂OMe | H | H | H | H | H | F | H | H |
| 6a | Me | H | Cl | H | H | H | H | H | H |
| 6b | CH₂Me | H | Cl | H | H | H | H | H | H |
| 6c | CH₂CHOH | H | Cl | H | H | H | H | H | H |
| 6d | CH₂CH₂OMe | H | Cl | H | H | H | H | H | H |
| 7a | Me | Cl | H | H | H | H | H | H | H |
| 7b | CH₂Me | Cl | H | H | H | H | H | H | H |
| 7c | CH₂CHOH | Cl | H | H | H | H | H | H | H |
| 7d | CH₂CH₂OMe | Cl | H | H | H | H | H | H | H |
| 8a | Me | H | H | H | H | Cl | H | H | H |
| 8b | CH₂Me | H | H | H | H | Cl | H | H | H |
| 8c | CH₂CHOH | H | H | H | H | Cl | H | H | H |
| 8d | CH₂CH₂OMe | H | H | H | H | Cl | H | H | H |
| 9a | Me | H | H | H | H | H | Cl | H | H |
| 9b | CH₂Me | H | H | H | H | H | Cl | H | H |
| 9c | CH₂CHOH | H | H | H | H | H | Cl | H | H |
| 9a | CH₂CH₂OMe | H | H | H | H | H | Cl | H | H |
| 10a | Me | H | H | H | H | Me | H | H | H |
| 10b | CH₂Me | H | H | H | H | Me | H | H | H |
| 10c | CH₂CHOH | H | H | H | H | Me | H | H | H |
| 10d | CH₂CH₂OMe | H | H | H | H | Me | H | H | H |
| 11a | Me | H | H | H | H | H | Me | H | H |
| 11b | CH₂Me | H | H | H | H | H | Me | H | H |
| 11c | CH₂CHOH | H | H | H | H | H | Me | H | H |
| 11d | CH₂CH₂OMe | H | H | H | H | H | Me | H | H |
| 12a | Me | H | OMe | H | H | H | H | H | H |
| 12b | CH₂Me | H | OMe | H | H | H | H | H | H |
| 12c | CH₂CHOH | H | OMe | H | H | H | H | H | H |
| 12d | CH₂CH₂OMe | H | OMe | H | H | H | H | H | H |
| 13a | Me | OMe | H | H | H | H | H | H | H |
| 13b | CH₂Me | OMe | H | H | H | H | H | H | H |
| 13c | CH₂CHOH | OMe | H | H | H | H | H | H | H |
| 13d | CH₂CH₂OMe | OMe | H | H | H | H | H | H | H |
| 14a | Me | H | H | H | H | OMe | H | H | H |
| 14b | CH₂Me | H | H | H | H | OMe | H | H | H |
| 14c | CH₂CHOH | H | H | H | H | OMe | H | H | H |
| 14d | CH₂CH₂OMe | H | H | H | H | OMe | H | H | H |
| 15a | Me | H | H | H | H | H | OMe | H | H |
| 15b | CH₂Me | H | H | H | H | H | OMe | H | H |
| 15c | CH₂CHOH | H | H | H | H | H | OMe | H | H |
| 15d | CH₂CH₂OMe | H | H | H | H | H | OMe | H | H |
| 16a | Me | H | CF₃ | H | H | H | H | H | H |
| 16b | CH₂Me | H | CF₃ | H | H | H | H | H | H |
| 16c | CH₂CHOH | H | CF₃ | H | H | H | H | H | H |
| 16d | CH₂CH₂OMe | H | CF₃ | H | H | H | H | H | H |
| 17a | Me | CF₃ | H | H | H | ? | H | H | H |
| 17b | CH₂Me | CF₃ | H | H | H | H | H | H | H |
| 17c | CH₂CHOH | CF₃ | H | H | H | H | H | H | H |
| 17d | CH₂CH₂OMe | CF₃ | H | H | H | H | H | H | H |
| 18a | Me | H | H | H | H | CF₃ | H | H | H |
| 18b | CH₂Me | H | H | H | H | CF₃ | H | H | H |
| 18c | CH₂CHOH | H | H | H | H | CF₃ | H | H | H |
| 18d | CH₂CH₂OMe | H | H | H | H | CF₃ | H | H | H |
| 19a | Me | H | H | H | H | H | CF₃ | H | H |
| 19b | CH₂Me | H | H | H | H | H | CF₃ | H | H |
| 19c | CH₂CHOH | H | H | H | H | H | CF₃ | H | H |
| 19d | CH₂CH₂OMe | H | H | H | H | H | CF₃ | H | H |
| 20 | Ethyl | H | Me | H | H | H | H | H | H |
| 21 | Ethyl | Me | H | H | H | H | H | H | H |

Compounds of the present invention include compounds having the formula (XXXIV) or a pharmaceutically acceptable salt form thereof:

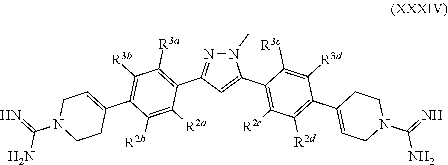

wherein non-limiting examples of R^{2a}, R^{2b}, R^{3a}, R^{3b}, R^{2c}, R^{2d}, R^{3c}, and R^{3d} are defined herein below in Table 23.

TABLE 23
Exemplary compounds of the formula (XXXIV)

| Entry | R^{2a} | R^{2b} | R^{3a} | R^{3b} | R^{2c} | R^{2d} | R^{3c} | R^{3d} |
|---|---|---|---|---|---|---|---|---|
| 1 | H | F | H | H | H | H | H | H |
| 2 | F | H | H | H | H | H | H | H |
| 3 | H | H | H | H | H | F | H | H |
| 4 | H | H | H | H | F | H | H | H |
| 5 | H | Cl | H | H | H | H | H | H |
| 6 | Cl | H | H | H | H | H | H | H |
| 7 | H | H | H | H | H | Cl | H | H |
| 8 | H | H | H | H | Cl | H | H | H |
| 9 | H | Me | H | H | H | H | H | H |
| 10 | Me | H | H | H | H | H | H | H |
| 11 | H | H | H | H | H | Me | H | H |
| 12 | H | H | H | H | Me | H | H | H |
| 13 | H | OMe | H | H | H | H | H | H |
| 14 | OMe | H | H | H | H | H | H | H |
| 15 | H | H | H | H | H | OMe | H | H |
| 16 | H | H | H | H | OMe | H | H | H |
| 17 | H | CF₃ | H | H | H | H | H | H |
| 18 | CF₃ | H | H | H | H | H | H | H |
| 19 | H | H | H | H | H | CF₃ | H | H |
| 20 | H | H | H | H | CF₃ | H | H | H |

Compounds of the present invention include compounds having the formula (XXXV) or a pharmaceutically acceptable salt form thereof:

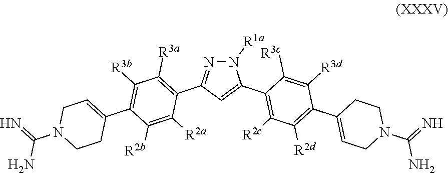

wherein non-limiting examples of R^{1a}, R^{2a}, R^{2b}, R^{3a}, R^{3b}, R^{2c}, R^{2d}, R^{3c}, and R^{3d} are defined herein below in Table 24.

TABLE 24
Exemplary compounds of the formula (XXXV)

| Entry | R^{1a} | R^{2ª} | R^{2b} | R^{3ª} | R^{3b} | R^{2c} | R^{2d} | R^{3c} | R^{3d} |
|---|---|---|---|---|---|---|---|---|---|
| 1 | Ethyl | H | H | H | H | H | H | H | H |
| 2 | Ethyl | H | F | H | H | H | H | H | H |
| 3 | Ethyl | F | H | H | H | H | H | H | H |
| 4 | Ethyl | H | H | H | H | H | F | H | H |
| 5 | Ethyl | H | H | H | H | F | H | H | H |
| 6 | Isopropyl | H | H | H | H | H | H | H | H |
| 7 | Isopropyl | H | H | H | H | F | H | H | H |
| 8 | Isopropyl | H | F | H | H | H | H | H | H |
| 9 | Isopropyl | F | H | H | H | H | H | H | H |
| 10 | Isopropyl | H | H | H | H | H | F | H | H |
| 11 | Cyclopropyl | H | H | H | H | H | H | H | H |
| 12 | Cyclopropyl | H | F | H | H | H | H | H | H |
| 13 | Cyclopropyl | H | H | H | H | F | H | H | H |
| 14 | Cyclopropyl | H | H | H | H | H | F | H | H |
| 15 | Cyclopropyl | F | H | H | H | H | H | H | H |
| 16 | 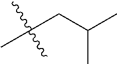 [SMILES: *CC(C)C] | H | H | H | H | F | H | H | H |
| 17 | 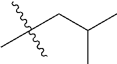 [SMILES: *CC(C)C] | H | H | H | H | H | H | H | H |
| 18 | 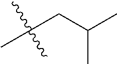 [SMILES: *CC(C)C] | H | F | H | H | H | H | H | H |
| 19 | 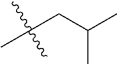 [SMILES: *CC(C)C] | F | H | H | H | H | H | H | H |
| 20 | 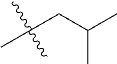 [SMILES: *CC(C)C] | H | H | H | H | H | F | H | H |
| 21 | 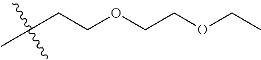 [SMILES: *CCOCCOCC] | H | H | H | H | F | H | H | H |
| 22 | 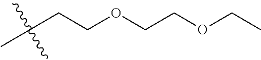 [SMILES: *CCOCCOCC] | H | H | H | H | H | H | H | H |
| 23 | 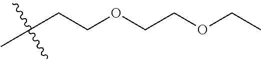 [SMILES: *CCOCCOCC] | H | F | H | H | H | H | H | H |
| 24 | 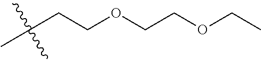 [SMILES: *CCOCCOCC] | F | H | H | H | H | H | H | H |
| 25 | 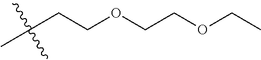 [SMILES: *CCOCCOCC] | H | H | H | H | H | F | H | H |
| 26 | n-hexyl | H | H | H | H | F | H | H | H |
| 27 | n-hexyl | H | H | H | H | H | H | H | H |
| 28 | n-hexyl | H | F | H | H | H | H | H | H |
| 29 | n-hexyl | F | H | H | H | H | H | H | H |
| 30 | n-hexyl | H | H | H | H | H | F | H | H |
| 31 | Benzyl | H | H | H | H | F | H | H | H |
| 32 | Benzyl | H | H | H | H | H | H | H | H |
| 33 | Benzyl | H | F | H | H | H | H | H | H |
| 34 | Benzy1 | F | H | H | H | H | H | H | H |
| 35 | Benzyl | H | H | H | H | H | F | H | H |

Compounds of the present invention include compounds having the formula (XXXVI) or a pharmaceutically acceptable salt form thereof:

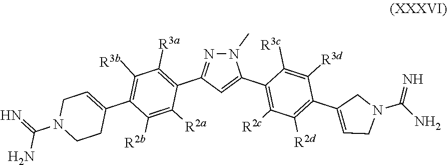

wherein non-limiting examples of R^{2a}, R^{2b}, R^{3a}, R^{3b}, R^{2c}, R^{2d}, R^{3c}, and R^{3d} are defined herein below in Table 25.

TABLE 25
Exemplary compounds of the formula (XXXVI)

| Entry | R^{2a} | R^{2b} | R³ª | R^{3b} | R^{2c} | R^{2d} | R^{3c} | R^{3d} |
|---|---|---|---|---|---|---|---|---|
| 1 | H | H | H | H | H | H | H | H |
| 2 | H | F | H | H | H | H | H | H |
| 3 | F | H | H | H | H | H | H | H |
| 4 | H | H | H | H | H | F | H | H |
| 5 | H | H | H | H | F | H | H | H |
| 6 | H | Cl | H | H | H | H | H | H |
| 7 | Cl | H | H | H | H | H | H | H |
| 8 | H | H | H | H | H | Cl | H | H |
| 9 | H | H | H | H | Cl | H | H | H |
| 10 | H | Me | H | H | H | H | H | H |
| 11 | Me | H | H | H | H | H | H | H |
| 12 | H | H | H | H | H | Me | H | H |
| 13 | H | H | H | H | Me | H | H | H |
| 14 | H | OMe | H | H | H | H | H | H |
| 15 | OMe | H | H | H | H | H | H | H |
| 16 | H | H | H | H | H | OMe | H | H |
| 17 | H | H | H | H | OMe | H | H | H |
| 18 | H | CF₃ | H | H | H | H | | H |
| 19 | CF₃ | H | H | H | H | H | H | H |
| 20 | H | H | H | H | H | CF₃ | H | H |
| 21 | H | H | H | H | CF₃ | H | H | H |

Compounds of the present invention include compounds having the formula (XXXVII) or a pharmaceutically acceptable salt form thereof:

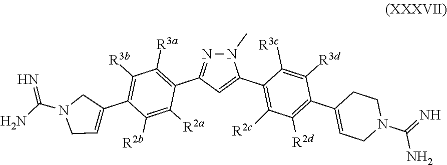

wherein non-limiting examples of R^{2a}, R^{2b}, R^{3a}, R^{3b}, R^{2c}, R^{2d}, R^{3c}, and R^{3d} are defined herein below in Table 26.

TABLE 26
Exemplary compounds of the formula (XXXVII)

| Entry | R^{2a} | R^{2b} | R³ª | R^{3b} | R^{2c} | R^{2d} | R^{3c} | R^{3d} |
|---|---|---|---|---|---|---|---|---|
| 1 | H | H | H | H | H | H | H | H |
| 2 | H | F | H | H | H | H | H | H |
| 3 | F | H | H | H | H | H | H | H |
| 4 | H | H | H | H | H | F | H | H |
| 5 | H | H | H | H | F | H | H | H |
| 6 | H | Cl | H | H | H | H | H | H |
| 7 | Cl | H | H | H | H | H | H | H |
| 8 | H | H | H | H | H | Cl | H | H |
| 9 | H | H | H | H | Cl | H | H | H |
| 10 | H | Me | H | H | H | H | H | H |
| 11 | Me | H | H | H | H | H | H | H |
| 12 | H | H | H | H | H | Me | H | H |
| 13 | H | H | H | H | Me | H | H | H |
| 14 | H | OMe | H | H | H | H | H | H |
| 15 | OMe | H | H | H | H | H | H | H |
| 16 | H | H | H | H | H | OMe | H | H |
| 17 | H | H | H | H | OMe | H | H | H |
| 18 | H | CF₃ | H | H | H | H | H | H |
| 19 | CF₃ | H | H | H | H | H | H | H |
| 20 | H | H | H | H | H | CF₃ | H | H |
| 21 | H | H | H | H | CF₃ | H | H | H |

Compounds of the present invention include compounds having the formula (XXXVIII) or a pharmaceutically acceptable salt form thereof:

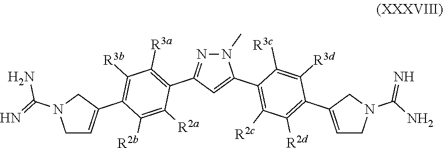

wherein non-limiting examples of R^{2a}, R^{2b}, R^{3a}, R^{3b}, R^{2c}, R^{2d}, R^{3c}, and R^{3d} are defined herein below in Table 27.

TABLE 27
Exemplary compounds of the formula (XXXVIII)

| Entry | R^{2a} | R^{2b} | R³ª | R^{3b} | R^{2c} | R^{2d} | R^{3c} | R^{3d} |
|---|---|---|---|---|---|---|---|---|
| 1 | H | H | H | H | H | H | H | H |
| 2 | H | F | H | H | H | H | H | H |
| 3 | F | H | H | H | H | H | H | H |
| 4 | H | H | H | H | H | F | H | H |
| 5 | H | H | H | H | F | H | H | H |
| 6 | H | Cl | H | H | H | H | H | H |
| 7 | Cl | H | H | H | H | H | H | H |
| 8 | H | H | H | H | H | Cl | H | H |
| 9 | H | H | H | H | Cl | H | H | H |
| 10 | H | Me | H | H | H | H | H | H |
| 11 | Me | H | H | H | H | H | H | H |
| 12 | H | H | H | H | H | Me | H | H |
| 13 | H | H | H | H | Me | H | H | H |
| 14 | H | OMe | H | H | H | H | H | H |
| 15 | OMe | H | H | H | H | H | H | H |
| 16 | H | H | H | H | H | OMe | H | H |
| 17 | H | H | H | H | OMe | H | H | H |
| 18 | H | CF₃ | H | H | H | H | H | H |
| 19 | CF₃ | H | H | H | H | H | H | H |
| 20 | H | H | H | H | H | CF₃ | H | H |
| 21 | H | H | H | H | CF₃ | H | H | H |

Compounds of the present invention include compounds having the formula (XXXIX) or a pharmaceutically acceptable salt form thereof:

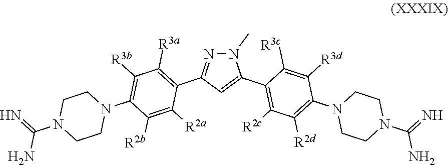

wherein non-limiting examples of R^{2a}, R^{2b}, R^{3a}, R^{3b}, R^{2c}, R^{2d}, R^{3c}, and R^{3d} are defined herein below in Table 28.

TABLE 28
Exemplary compounds of the formula (XXXIX)

| Entry | R^{2a} | R^{2b} | R³ª | R^{3b} | R^{2c} | R^{2d} | R^{3c} | R^{3d} |
|---|---|---|---|---|---|---|---|---|
| 1 | H | F | H | H | H | H | H | H |
| 2 | F | H | H | H | H | H | H | H |
| 3 | H | H | H | H | H | F | H | H |
| 4 | H | H | H | H | F | H | H | H |
| 5 | H | Cl | H | H | H | H | H | H |
| 6 | Cl | H | H | H | H | H | H | H |
| 7 | H | H | H | H | H | Cl | H | H |
| 8 | H | H | H | H | Cl | H | H | H |
| 9 | H | Me | H | H | H | H | H | H |
| 10 | Me | H | H | H | H | H | H | H |
| 11 | H | H | H | H | H | Me | H | H |
| 12 | H | H | H | H | Me | H | H | H |
| 13 | H | OMe | H | H | H | H | H | H |
| 14 | OMe | H | H | H | H | H | H | H |
| 15 | H | H | H | H | H | OMe | H | H |
| 16 | H | H | H | H | OMe | H | H | H |
| 17 | H | CF₃ | H | H | H | H | H | H |
| 18 | CF₃ | H | H | H | H | H | H | H |
| 19 | H | H | H | H | H | CF₃ | H | H |
| 20 | H | H | H | H | CF₃ | H | H | H |

Compounds of the present invention include compounds having the formula (XXXX) or a pharmaceutically acceptable salt form thereof:

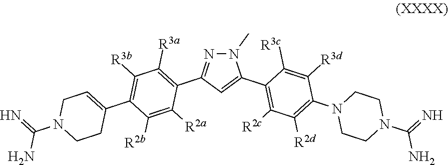

wherein non-limiting examples of R^{2a}, R^{2b}, R^{3a}, R^{3b}, R^{2c}, R^{2d}, R^{3c}, and R^{3d} are defined herein below in Table 29.

TABLE 29
Exemplary compounds of the formula (XXXX)

| Entry | R^{2a} | R^{2b} | R³ª | R^{3b} | R^{2c} | R^{2d} | R^{3c} | R^{3d} |
|---|---|---|---|---|---|---|---|---|
| 1 | H | H | H | H | H | H | H | H |
| 2 | H | F | H | H | H | H | H | H |
| 3 | F | H | H | H | H | H | H | H |
| 4 | H | H | H | H | H | F | H | H |
| 5 | H | H | H | H | F | H | H | H |
| 6 | H | Cl | H | H | H | H | H | H |
| 7 | Cl | H | H | H | H | H | H | H |
| 8 | H | H | H | H | H | Cl | H | H |
| 9 | H | H | H | H | Cl | H | H | H |
| 10 | H | Me | H | H | H | H | H | H |
| 11 | Me | H | H | H | H | H | H | H |
| 12 | H | H | H | H | H | Me | H | H |
| 13 | H | H | H | H | Me | H | H | H |
| 14 | H | OMe | H | H | H | H | H | H |
| 15 | OMe | H | H | H | H | H | H | H |
| 16 | H | H | H | H | H | OMe | H | H |
| 17 | H | H | H | H | OMe | H | H | H |
| 18 | H | CF₃ | H | H | H | H | H | H |
| 19 | CF₃ | H | H | H | H | H | H | H |
| 20 | H | H | H | H | H | CF₃ | H | H |
| 21 | H | H | H | H | CF₃ | H | H | H |

Compounds of the present invention include compounds having the formula (XXXXI) or a pharmaceutically acceptable salt form thereof:

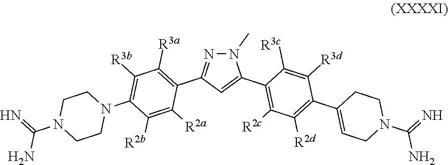

wherein non-limiting examples of R^{2a}, R^{2b}, R^{3a}, R^{3b}, R^{2c}, R^{2d}, R^{3c}, and R^{3d} are defined herein below in Table 30.

TABLE 30
Exemplary compounds of the formula (XXXXI)

| Entry | R^{2a} | R^{2b} | R³ª | R^{3b} | R^{2c} | R^{2d} | R^{3c} | R^{3d} |
|---|---|---|---|---|---|---|---|---|
| 1 | H | H | H | H | H | H | H | H |
| 2 | H | F | H | H | H | H | H | H |
| 3 | F | H | H | H | H | H | H | H |
| 4 | H | H | H | H | H | F | H | H |
| 5 | H | H | H | H | F | H | H | H |
| 6 | H | Cl | H | H | H | H | H | H |
| 7 | Cl | H | H | H | H | H | H | H |
| 8 | H | H | H | H | H | Cl | H | H |
| 9 | H | H | H | H | Cl | H | H | H |
| 10 | H | Me | H | H | H | H | H | H |
| 11 | Me | H | H | H | H | H | H | H |
| 12 | H | H | H | H | H | Me | H | H |
| 13 | H | H | H | H | Me | H | H | H |
| 14 | H | OMe | H | H | H | H | H | H |
| 15 | OMe | H | H | H | H | H | H | H |
| 16 | H | H | H | H | H | OMe | H | H |
| 17 | H | H | H | H | OMe | H | H | H |
| 18 | H | CF₃ | H | H | H | H | H | H |
| 19 | CF₃ | H | H | H | H | H | H | H |
| 20 | H | H | H | H | H | CF₃ | H | H |
| 21 | H | H | H | H | CF₃ | H | H | H |

Compounds of the present invention include compounds having the formula (XXXXII) or a pharmaceutically acceptable salt form thereof:

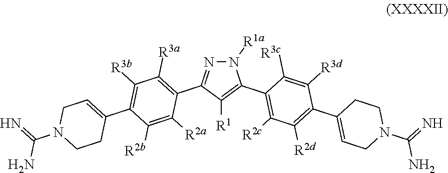

wherein non-limiting examples of R¹, R^{1a}, R^{2a}, R^{2b}, R^{3a}, R^{3b}, R^{2c}, R^{2d}, R^{3c}, and R^{3d} are defined herein

TABLE 31
Exemplary compounds of the formula (XXXXII)

| Entry | R¹ | R^{1a} | R^{2a} | R^{2b} | R³ª | R^{3b} | R^{2c} | R^{2d} | R^{3c} | R^{3d} |
|---|---|---|---|---|---|---|---|---|---|---|
| 1a | Me | Me | H | H | H | H | H | H | H | H |
| 1b | Me | CH₂Me | H | H | H | H | H | H | H | H |
| 1c | Me | CH₂CHOH | H | H | H | H | H | H | H | H |
| ld | Me | CH₂CH₂OMe | H | H | H | H | H | H | H | H |
| 2a | Me | Me | H | F | H | H | H | H | H | H |
| 2b | Me | CH₂Me | H | F | H | H | H | H | H | H |
| 2c | Me | CH₂CHOH | H | F | H | H | H | H | H | H |
| 2d | Me | CH₂CH₂OMe | H | F | H | H | H | H | H | H |
| 3a | Me | Me | F | H | H | H | H | H | H | H |
| 3b | Me | CH₂Me | F | H | H | H | H | H | H | H |
| 3c | Me | CH₂CHOH | F | H | H | H | H | H | H | H |
| 3d | Me | CH₂CH₂OMe | F | H | H | H | H | H | H | H |
| 4a | Me | Me | H | H | H | H | H | F | H | H |
| 4b | Me | CH₂Me | H | H | H | H | H | F | H | H |
| 4c | Me | CH₂CHOH | H | H | H | H | H | F | H | H |
| 4d | Me | CH₂CH₂OMe | H | H | H | H | H | F | H | H |
| 5a | Me | Me | H | H | H | H | F | H | H | H |
| 5b | Me | CH₂Me | H | H | H | H | F | H | H | H |
| 5c | Me | CH₂CHOH | H | H | H | H | F | H | H | H |
| 5d | Me | CH₂CH₂OMe | H | H | H | H | F | H | H | H |
| 6a | Me | Me | H | Cl | H | H | H | H | H | H |
| 6b | Me | CH₂Me | H | Cl | H | H | H | H | H | H |
| 6c | Me | CH₂CHOH | H | Cl | H | H | H | H | H | H |
| 6d | Me | CH₂CH₂OMe | H | Cl | H | H | H | H | H | H |
| 7a | Me | Me | Cl | H | H | H | H | H | H | H |
| 7b | Me | CH₂Me | Cl | H | H | H | H | H | H | H |
| 7c | Me | CH₂CHOH | Cl | H | H | H | H | H | H | H |
| 7d | Me | CH₂CH₂OMe | Cl | H | H | H | H | H | H | H |
| 8a | Me | Me | H | H | H | H | H | Cl | H | H |
| 8b | Me | CH₂Me | H | H | H | H | H | Cl | H | H |
| 8c | Me | CH₂CHOH | H | H | H | H | H | Cl | H | H |
| 8d | Me | CH₂CH₂OMe | H | H | H | H | H | Cl | H | H |
| 9a | Me | Me | H | H | H | H | Cl | H | H | H |
| 9b | Me | CH₂Me | H | H | H | H | Cl | H | H | H |
| 9c | Me | CH₂CHOH | H | H | H | H | Cl | H | H | H |
| 9d | Me | CH₂CH₂OMe | H | H | H | H | Cl | H | H | H |
| 10a | Me | Me | H | Me | H | H | H | H | H | H |
| 10b | Me | CH₂Me | H | Me | H | H | H | H | H | H |
| 10c | Me | CH₂CHOH | H | Me | H | H | H | H | H | H |
| 10d | Me | CH₂CH₂OMe | H | Me | H | H | H | H | H | H |
| 11a | Me | Me | Me | H | H | H | H | H | H | H |
| 11b | Me | CH₂Me | Me | H | H | H | H | H | H | H |
| 11c | Me | CH₂CHOH | Me | H | H | H | H | H | H | H |
| 1ld | Me | CH₂CH₂OMe | Me | H | H | H | H | H | H | H |
| 12a | Me | Me | H | H | H | H | H | Me | H | H |
| 12b | Me | CH₂Me | H | H | H | H | H | Me | H | H |
| 12c | Me | CH₂CHOH | H | H | H | TH | H | Me | H | H |
| 12d | Me | CH₂CH₂OMe | H | H | H | H | H | Me | H | H |
| 13a | Me | Me | H | H | H | H | Me | H | H | H |
| 13b | Me | CH₂Me | H | H | H | H | Me | H | H | H |
| 13c | Me | CH₂CHOH | H | H | H | H | Me | H | H | H |
| 13d | Me | CH₂CH₂OMe | H | H | H | H | Me | H | H | H |
| 14a | Me | Me | H | OMe | H | H | H | H | H | H |
| 14b | Me | CH₂Me | H | OMe | H | H | H | H | H | H |
| 14c | Me | CH₂CHOH | H | OMe | H | H | H | H | H | H |
| 14d | Me | CH₂CH₂OMe | H | OMe | H | H | H | H | H | H |
| 15a | Me | Me | OMe | H | H | H | H | H | H | H |
| 15b | Me | CH₂Me | OMe | H | H | H | H | H | H | H |
| 15c | Me | CH₂CHOH | OMe | H | H | H | H | H | H | H |
| 15d | Me | CH₂CH₂OMe | OMe | H | H | H | H | H | H | H |
| 16a | Me | Me | H | H | H | H | H | OMe | H | H |
| 16b | Me | CH₂Me | H | H | H | H | H | OMe | H | H |
| 16c | Me | CH₂CHOH | H | H | H | H | H | OMe | H | H |
| 16d | Me | CH₂CH₂OMe | H | H | H | H | H | OMe | H | H |
| 17a | Me | Me | H | H | HH | | OMe | H | H | H |
| 17b | Me | CH₂Me | H | H | H | 1 H | OMe | H | H | H |
| 17c | Me | CH₂CHOH | H | H | H | IH | OMe | H | H | H |
| 17c | Me | CH₂CH₂OMe | H | H | H | H | OMe | H | H | H |
| 18a | Me | Me | H | CF₃ | H | H | H | H | H | H |
| 18b | Me | CH₂Me | H | CF₃ | H | H | H | H | H | H |
| 18c | Me | CH₂CHOH | H | CF₃ | H | H | H | H | H | H |
| 18d | Me | CH₂CH₂OMe | H | CF₃ | H | H | H | H | H | H |
| 19a | Me | Me | CF₃ | H | H | H | H | H | H | H |
| 19b | Me | CH₂Me | CF₃ | H | H | H | H | H | H | H |
| 19c | Me | CH₂CHOH | CF₃ | H | H | TH | H | H | H | H |
| 19d | Me | CH₂CH₂OMe | CF₃ | H | H | H | H | H | H | H |
| 20a | Me | Me | H | H | H | H | H | CF₃ | H | H |
| 20b | Me | CH₂Me | H | H | H | H | H | CF₃ | H | H |
| 20c | Me | CH₂CHOH | H | H | H | H | H | CF₃ | H | H |
| 20d | Me | CH₂CH₂OMe | H | H | H | H | H | CF₃ | H | H |
| 21a | Me | Me | H | H | H | H | CF₃ | H | H | H |
| 21b | Me | CH₂Me | H | H | H | H | CF₃ | H | H | H |
| 21c | Me | CH₂CHOH | H | H | H | H | CF₃ | H | H | H |
| 21d | Me | CH₂CH₂OMe | H | H | H | H | CF₃ | H | H | H |

Compounds of the present invention include compounds having the formula (XXXXIII) or a pharmaceutically acceptable salt form thereof:

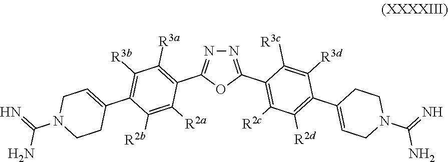

wherein non-limiting examples of R^{2a}, R^{2b}, R^{3a}, R^{3b}, R^{2c}, R^{2d}, R^{3c}, and R^{3d} are defined herein below in Table 32.

TABLE 32
Exemplary compounds of the formula (XXXXIII)

| Entry | R^{2a} | R^{2b} | R³ª | R^{3b} | R^{2c} | R^{2d} | R^{3c} | R^{3d} |
|---|---|---|---|---|---|---|---|---|
| 1 | H | F | H | H | H | H | H | H |
| 2 | H | Cl | H | H | H | H | H | H |
| 3 | 6 | H | H | H | H | H | H | H |
| 4 | H | Cl | H | H | H | Cl | H | H |
| 5 | Cl | H | H | H | Cl | H | H | H |
| 6 | H | Me | H | H | H | Me | H | H |
| 7 | Me | H | H | H | Me | H | H | H |
| 8 | H | OMe | H | H | H | H | H | H |
| 9 | OMe | H | H | H | H | H | H | H |
| 10 | H | OMe | H | H | H | OMe | H | H |
| 11 | OMe | H | H | H | OMe | H | H | H |
| 12 | H | CF₃ | H | H | H | CF₃ | H | H |
| 13 | CF₃ | H | H | H | CF₃ | H | H | H |

Compounds of the present invention include compounds having the formula (XXXXIV) or a pharmaceutically acceptable salt form thereof:

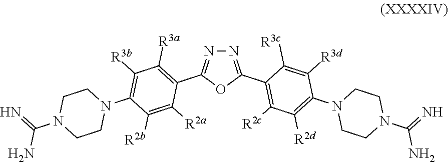

wherein non-limiting examples of R^{2a}, R^{2b}, R^{3a}, R^{3b}, R^{2c}, R^{2d}, R^{3c}, and R^{3d} are defined herein below in Table 33.

TABLE 33
Exemplary compounds of the formula (XXXXIV)

| Entry | R^{2a} | R^{2b} | R³ª | R^{3b} | R^{2c} | R^{2d} | R^{3c} | R^{3d} |
|---|---|---|---|---|---|---|---|---|
| 1 | H | F | H | H | H | H | H | H |
| 2 | F | H | H | H | H | H | H | H |
| 3 | H | F | H | H | H | F | H | H |
| 4 | F | H | H | H | F | H | H | H |
| 5 | H | Cl | H | H | H | H | H | H |
| 6 | Cl | H | H | H | H | H | H | H |
| 7 | H | Cl | H | H | H | Cl | H | H |
| 8 | Cl | H | H | H | Cl | H | H | H |
| 9 | H | Me | H | H | H | Me | H | H |
| 10 | Me | H | H | H | Me | H | H | H |
| 11 | H | OMe | H | H | H | H | H | H |
| 12 | OMe | H | H | H | H | H | H | H |
| 13 | H | OMe | H | H | H | OMe | H | H |
| 14 | OMe | H | H | H | OMe | H | H | H |
| 15 | H | CF₃ | H | H | H | H | H | H |
| 16 | CF₃ | H | H | H | H | H | H | H |
| 17 | H | CF₃ | H | H | H | CF₃ | H | H |
| 18 | CF₃ | H | H | H | CF₃ | H | H | H |

Compounds of the present invention include compounds having the formula (XXXXV) or a pharmaceutically acceptable salt form thereof:

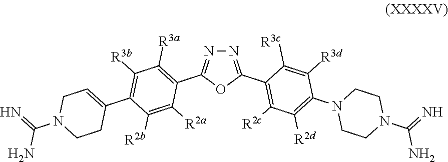

wherein non-limiting examples of R^{2a}, R^{2b}, R^{3a}, R^{3b}, R^{2c}, R^{2d}, R^{3c}, and R^{3d} are defined herein below in Table 34.

TABLE 34
Exemplary compounds of the formula (XXXXV)

| Entry | R^{2a} | R^{2b} | R³ª | R^{3b} | R^{2c} | R^{2d} | R^{3c} | R^{3d} |
|---|---|---|---|---|---|---|---|---|
| 1 | H | H | H | H | H | H | H | H |
| 2 | H | F | H | H | H | H | H | H |
| 3 | F | H | H | H | H | H | H | H |
| 4 | H | F | H | H | H | F | H | H |
| 5 | F | H | H | H | F | H | H | H |
| 6 | H | Cl | H | H | B | H | H | H |
| 7 | Cl | H | H | H | H | H | H | H |
| 8 | H | Cl | H | H | H | Cl | H | H |
| 9 | Cl | H | H | H | Cl | H | H | H |
| 10 | H | Me | H | H | H | H | H | H |
| 11 | Me | H | H | H | H | H | H | H |
| 12 | H | Me | H | H | H | Me | H | H |
| 13 | Me | H | H | H | Me | H | H | H |
| 14 | H | OMe | H | H | H | H | H | H |
| 15 | OMe | H | H | H | H | H | H | H |
| 16 | H | OMe | H | H | H | OMe | H | H |
| 17 | OMe | H | H | H | OMe | H | H | H |
| 18 | H | CF₃ | H | H | H | H | H | H |
| 19 | CF₃ | H | H | H | H | H | H | H |
| 20 | H | CF₃ | H | H | H | CF₃ | H | H |
| 21 | CF₃ | H | H | H | CF₃ | H | H | H |

Compounds of the present invention include compounds having the formula (XXXXVI) or a pharmaceutically acceptable salt form thereof:

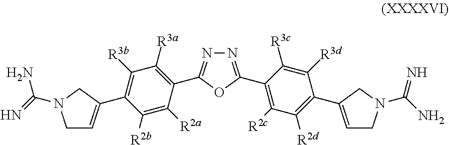

wherein non-limiting examples of R^{2a}, R^{2b}, R^{3a}, R^{3b}, R^{2c}, R^{2d}, R^{3c}, and R^{3d} are defined herein below in Table 35.

TABLE 35
Exemplary compounds of the formula (XXXXVI)

| Entry | R^{2a} | R^{2b} | R³ª | R^{3b} | R^{2c} | R^{2d} | R^{3c} | R^{3d} |
|---|---|---|---|---|---|---|---|---|
| 1 | H | H | H | H | H | H | H | H |
| 2 | H | F | H | H | H | H | H | H |
| 3 | F | H | H | H | H | H | H | H |
| 4 | H | F | H | H | H | F | H | H |
| 5 | F | H | H | H | F | H | H | H |
| 6 | H | Cl | H | H | H | H | H | H |
| 7 | Cl | H | H | H | H | H | H | H |
| 8 | H | Cl | H | H | H | Cl | H | H |
| 9 | Cl | H | H | H | Cl | H | H | H |
| 10 | H | Me | H | H | H | H | H | H |
| 11 | Me | H | H | H | H | H | H | H |
| 12 | H | Me | H | H | H | Me | H | H |
| 13 | Me | H | H | H | Me | H | H | H |
| 14 | H | OMe | H | H | H | H | H | H |
| 15 | OMe | H | H | H | H | H | H | H |
| 16 | H | OMe | H | H | H | OMe | H | H |
| 17 | OMe | H | H | H | OMe | H | H | h |
| 18 | CF₃ | H | H | H | H | H | H | H |
| 19 | H | CF₃ | H | H | H | CF₃ | H | H |
| 20 | CF₃ | H | H | H | CF₃ | H | H | H |

Compounds of the present invention include compounds having the formula (XXXXVII) or a pharmaceutically acceptable salt form thereof:

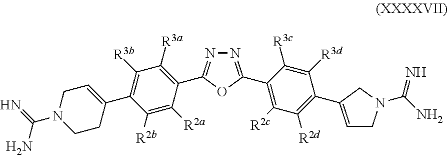

wherein non-limiting examples of R^{2a}, R^{2b}, R^{3a}, R^{3b}, R^{2c}, R^{2d}, R^{3c}, and R^{3d} are defined herein below in Table 36.

TABLE 36
Exemplary compounds of the formula (XXXXVII)

| Entry | R^{2a} | R^{2b} | R³ª | R^{3b} | R^{2c} | R^{2d} | R^{3c} | R^{3d} |
|---|---|---|---|---|---|---|---|---|
| 1 | H | H | H | H | H | H | H | H |
| 2 | H | F | H | H | H | H | H | H |
| 3 | F | H | H | H | H | H | H | H |
| 4 | H | F | H | H | H | F | H | H |
| 5 | F | H | H | H | F | H | H | H |
| 6 | H | Cl | H | H | H | H | H | H |
| 7 | Cl | H | H | H | H | H | H | H |
| 8 | H | Cl | H | H | H | Cl | H | H |
| 9 | Cl | H | H | H | Cl | H | H | H |
| 10 | H | Me | H | H | H | H | H | H |
| 11 | Me | H | H | H | H | H | H | H |
| 12 | H | Me | H | H | H | Me | H | H |
| 13 | Me | H | H | H | Me | H | H | H |
| 14 | H | OMe | H | H | H | H | H | H |
| 15 | OMe | H | H | H | H | H | H | H |
| 16 | H | OMe | H | H | H | OMe | H | H |
| 17 | OMe | H | H | H | OMe | H | H | H |
| 18 | H | CF₃ | H | H | H | H | H | H |
| 19 | CF₃ | H | H | H | H | H | H | H |
| 20 | H | CF₃ | H | H | H | CF₃ | H | H |
| 21 | CF₃ | H | H | H | CF₃ | H | H | H |

For the purposes of demonstrating the manner in which the compounds of the present invention are named and referred to herein, the compound having the formula:

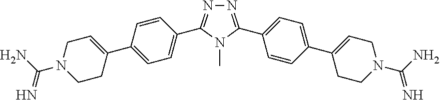 [SMILES: Cn1c(-c2ccc(C3=CCN(C(=N)N)CC3)cc2)nnc1-c1ccc(C2=CCN(C(=N)N)CC2)cc1]

has the chemical name 4,4′-(4,4′-(4-Methyl-4H-1,2,4-triazole-3,5-diyl)bis(4,1-phenylene))bis(5,6-dihydropyridine-1(2H)-carboximidamide).

For the purposes of the present invention, a compound depicted by the racemic formula will stand equally well for either of the two enantiomers or mixtures thereof, or in the case where a second chiral center is present, all diastereomers.

In all of the embodiments provided herein, examples of suitable optional substituents are not intended to limit the scope of the claimed invention. The compounds of the invention may contain any of the substituents, or combinations of substituents, provided herein.

### Process

The present invention further relates to a process for preparing the antifungal effect agents of the present invention.

Compounds of the present teachings can be prepared in accordance with the procedures outlined herein, from commercially available starting materials, compounds known in the literature, or readily prepared intermediates, by employing standard synthetic methods and procedures known to those skilled in the art. Standard synthetic methods and procedures for the preparation of organic molecules and functional group transformations and manipulations can be readily obtained from the relevant scientific literature or from standard textbooks in the field. It will be appreciated that where typical or preferred process conditions (i.e., reaction temperatures, times, mole ratios of reactants, solvents, pressures, etc.) are given, other process conditions can also be used unless otherwise stated. Optimum reaction conditions can vary with the particular reactants or solvent used, but such conditions can be determined by one skilled in the art by routine optimization procedures. Those skilled in the art of organic synthesis will recognize that the nature and order of the synthetic steps presented can be varied for the purpose of optimizing the formation of the compounds described herein.

The processes described herein can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), mass spectrometry, or by chromatography such as high pressure liquid chromatography (HPLC), gas chromatography (GC), gel-permeation chromatography (GPC), or thin layer chromatography (TLC).

Preparation of the compounds can involve protection and deprotection of various chemical groups. The need for protection and deprotection and the selection of appropriate protecting groups can be readily determined by one skilled in the art. The chemistry of protecting groups can be found, for example, in Greene et al., Protective Groups in Organic Synthesis, 2d. Ed. (Wiley & Sons, 1991), the entire disclosure of which is incorporated by reference herein for all purposes.

The reactions or the processes described herein can be carried out in suitable solvents which can be readily selected by one skilled in the art of organic synthesis. Suitable solvents typically are substantially nonreactive with the reactants, intermediates, and/or products at the temperatures at which the reactions are carried out, i.e., temperatures that can range from the solvent's freezing temperature to the solvent's boiling temperature. A given reaction can be carried out in one solvent or a mixture of more than one solvent. Depending on the particular reaction step, suitable solvents for a particular reaction step can be selected.

The compounds of these teachings can be prepared by methods known in the art of organic chemistry. The reagents used in the preparation of the compounds of these teachings can be either commercially obtained or can be prepared by standard procedures described in the literature. For example, compounds of the present invention can be prepared according to the method illustrated in the General Synthetic Schemes.

General Synthetic Schemes for Preparation of Compounds.

The reagents used in the preparation of the compounds of this invention can be either commercially obtained or can be prepared by standard procedures described in the literature. In accordance with this invention, compounds in the genus may be produced by one of the following reaction schemes.

Compounds of formula (I) may be prepared according to the process outlined in schemes 1-67.

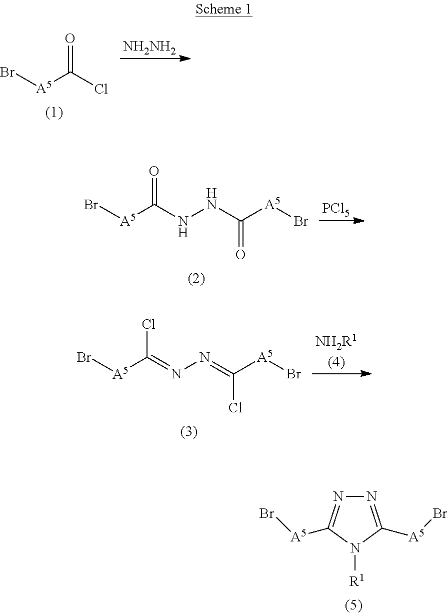

A compound of the formula (1), a known compound or a compound prepared by known methods, is reacted with hydrazine in the presence of a base such as triethylamine, N,N-diisopropylethylamine, pyridine, 2,6-dimethylpyridine, and the like, in the presence of a solvent such as methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (2). A compound of the formula (2) is reacted with phosphorous pentachloride in the presence of a solvent such as benzene, toluene, xylene, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (3). A compound of the formula (3) is reacted with a compound of the formula (4), a known compound or a compound prepared by known methods, in the presence of a solvent such as methanol, ethanol, isopropanol, methylene chloride, chloroform, 1,2-dichloroethane, tetrahydrofuran, 1,4-dioxane, acetonitrile, 1,2-dimethoxyethane, and the like, optionally in the presence of a base such as triethylamine, N,N-diisopropylethylamine, pyridine, 2,6-dimethylpyridine, and the like, optionally with heating, optionally with microwave irradiation to provide a compound of the formula (5).

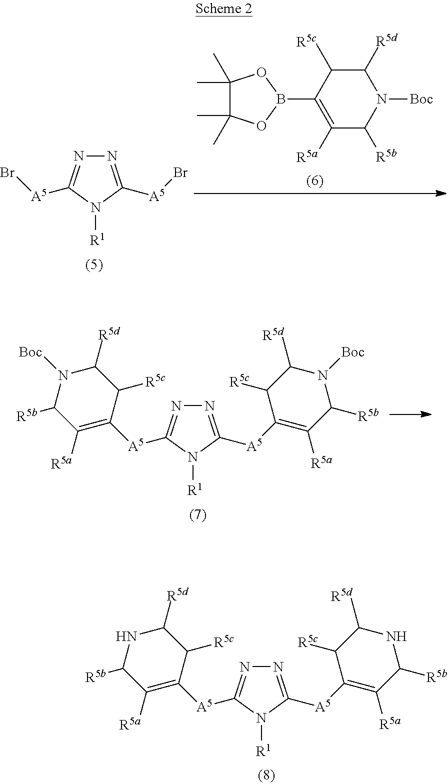

A compound of the formula (5) is reacted with a compound of the formula (6), a known compound or a compound prepared by known methods, in the presence of a palladium catalyst such as palladium (II) acetate, tetrakis(triphenylphosphine)palladiunn(0), dichlorobis (triphenylphosphine) palladium(II), palladium on carbon, bis(acetonitrile)dichloropalladium(II), and the like, in the presence of an organophosphine such as 2-dicyclohexylphosphino-2′,6′-dimethoxybiphenyl, 2-dicyclohexylphosphino-2′-(N,N-dimethylamino)biphenyl, 2-dicyclohexylphosphino-2′,4′,6′-triisopropylbiphenyl, 2-di-tert-butylphosphino-2′,4′,6′-triisopropylbiphenyl, (2-biphenyl)dicyclohexyl phosphine, (2-biphenyl)di-tert-butylphosphine, 2-dicyclohexylphosphino-2′,6′-diisopropoxybiphenyl, 2-di-tert-butylphosphino-3,4,5,6-tetra methyl-2′,4′,6′-triisopropyl-1,1′-biphenyl, Sodium 2′-dicyclohexylphosphino-2,6-dimethoxy-1,1′-biphenyl-3-sulfonate, 2-di-tert-butylphosphino-2′-methyl biphenyl, 2-dicyclohexylphosphino-2′-methylbiphenyl, 2′-(di-tert-butylphosphino)-N,N-dimethyl biphenyl-2-amine, 2′-(diphenylphosphino)-N,N′-dimethyl-(1,1′-biphenyl)-2-amine, and the like, in the presence of a base such as sodium carbonate, lithium carbonate, potassium carbonate, cesium carbonate, sodium hydroxide, lithium hydroxide, potassium hydroxide, triethylamine, N,N-diisopropylethylamine, pyridine, 2,6-dimethylpyridine, and the like, in a solvent such as tetrahydrofuran, 1,4-dioxane, acetonitrile, methylene chloride, chloroform, 1,2-dichloroethane, 1,2-dimethoxyethane, and the like, optionally with heating, optionally with microwave irradiation to provide a compound of the formula (7). A compound of the formula (7) is reacted with an acid such as trifluoroacetic acid, hydrochloric acid, sulfuric acid, and the like, in a solvent such as methylene chloride, 1,2-dichloroethane, tetrahydrofuran, 1,4-dioxane, methanol, ethanol, N,N-dimethylformamide, dimethylsulfoxide, N,N-dimethylacetamide, and the like, optionally in the presence of methylphenyl ether, optionally with heating, optionally with microwave irradiation to provide a compound of the formula (8).

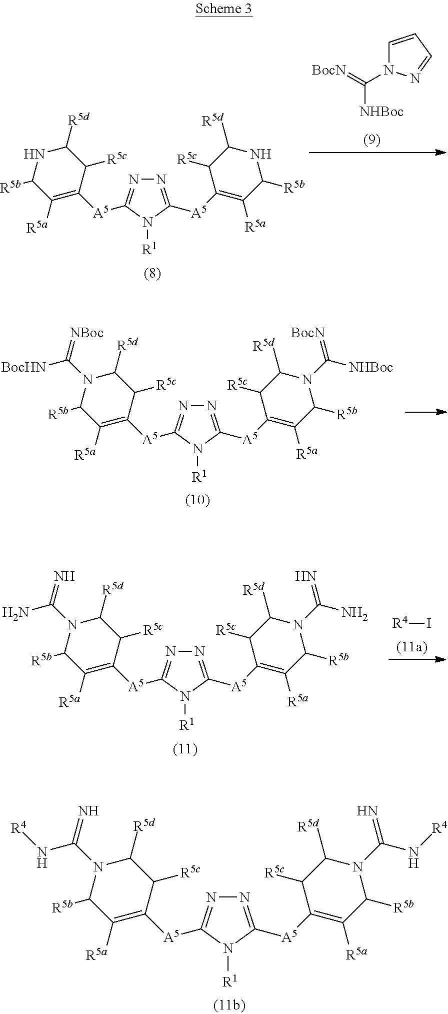

A compound of the formula (8) is reacted with a compound of the formula (9) in the presence of a base such as triethylamine, N,N-diisopropylethylamine, pyridine, 2,6-dimethylpyridine, and the like, in the presence of a solvent such as N,N-dimethylformamide, dimethylsulfoxide, N,N-dimethylacetamide, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (10). A compound of the formula (10) is reacted with an acid such as trifluoroacetic acid, hydrochloric acid, sulfuric acid, and the like, in a solvent such as methylene chloride, chloroform, 1,2-dichloroethane, tetrahydrofuran, 1,4-dioxane, methanol, ethanol, N,N-dimethylformamide, dimethylsulfoxide, N,N-dimethylacetamide, and the like, optionally in the presence of methylphenyl ether, optionally with heating, optionally with microwave irradiation to provide a compound of the formula (11). A compound of the formula (11) is reacted with a compound of the formula (11a), a known compound or a compound prepared by known methods, in the presence of a base such as sodium carbonate, potassium carbonate, lithium carbonate, triethylamine, pyridine, and the like, in a solvent such as N,N-dimethylformamide, dimethylsulfoxide, N,N-dimethylacetamide, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (11b).

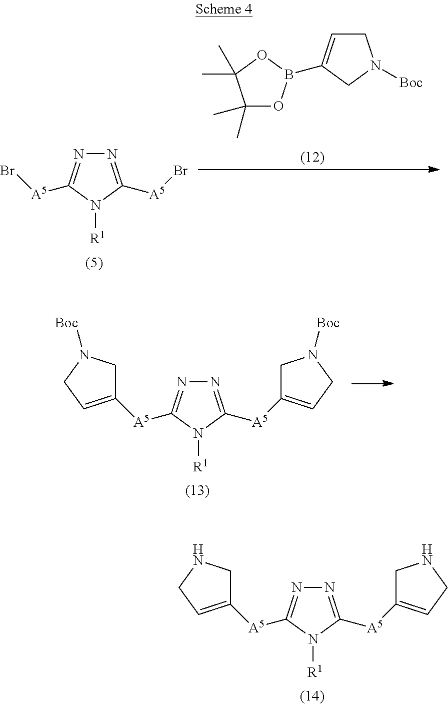

A compound of the formula (5) is reacted with a compound of the formula (12), a known compound or a compound prepared by known methods, in the presence of a palladium catalyst such as palladium. (ii) acetate, tetrakis(triphenylphosphine)palladium(0), dichlorobis (triphenylphosphine) palladium(II), palladium on carbon, bis(acetonitrile)dichloropalladium(II), and the like, in the presence of an organophosphine such as 2-dicyclohexylphosphino-2′,6′-dimethoxybiphenyl, 2-dicyclohexylphosphino-2′-(N,N-dimethylamino)biphenyl, 2-dicyclohexylphosphino-2′,4′,6′-triisopropylbiphenyl, 2-di-tert-butylphosphino-2′,4′,6′-triisopropylbiphenyl, (2-biphenyl)dicyclohexyl phosphine, (2-biphenyl)di-tert-butylphosphine, 2-dicyclohexylphosphino-2′,6′-diisopropoxybiphenyl, 2-di-tert-butylphosphino-3,4,5,6-tetramethyl-2′,4′,6′-triisopropyl-1,1′-biphenyl, Sodium 2′-dicyclohexylphosphino-2,6-dimethoxy-1,1′-biphenyl-3-sulfonate, 2-di-tert-butylphosphino-T-methyl biphenyl, 2-dicyclohexylphosphino-2′-methylbiphenyl, 2′-(di-tert-butylphosphino)-N,N-dimethyl biphenyl-2-amine, 2′-(diphenylphosphino)-N,N′-dimethyl-(1,1′-biphenyl)-2-amine, and the like, in the presence of a base such as sodium carbonate, lithium carbonate, potassium carbonate, cesium carbonate, sodium hydroxide, lithium hydroxide, potassium hydroxide, triethylamine, N,N-diisopropylethylamine, pyridine, 2,6-dimethylpyridine, and the like, in a solvent such as tetrahydrofuran, 1,4-dioxane, acetonitrile, methylene chloride, chloroform, 1,2-dichloroethane, 1,2-dimethoxyethane, and the like, optionally with heating, optionally with microwave irradiation to provide a compound of the formula (13). A compound of the formula (13) is reacted with an acid such as trifluoroacetic acid, hydrochloric acid, sulfuric acid, and the like, in a solvent such as methylene chloride, 1,2-dichloroethane, tetrahydrofuran, 1,4-dioxane, methanol, ethanol, N,N-dimethylformamide, dimethylsulfoxide, N,N-dimethylacetamide, and the like, optionally in the presence of methylphenyl ether, optionally with heating, optionally with microwave irradiation to provide a compound of the formula (14).

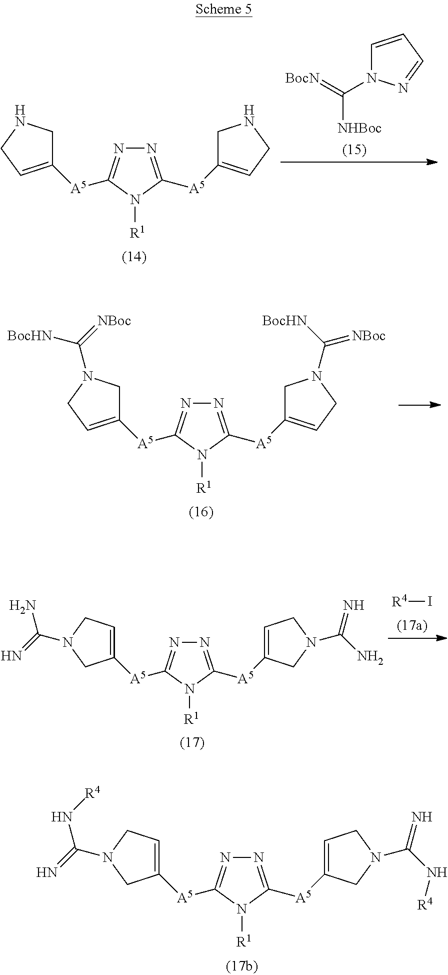

A compound of the formula (14) is reacted with a compound of the formula (15) in the presence of a base such as triethylamine, N,N-diisopropylethylamine, pyridine, 2,6-dimethylpyridine, and the like, in the presence of a solvent such as N,N-dimethylformamide, dimethylsulfoxide, N,N-dimethylacetamide, methylene chloride, 1,2-dichloroethane, tetrahydrofuran, 1,4-dioxane, acetonitrile, 1,2-dimethoxyethane, and the like, optionally with heating, optionally with microwave irradiation to provide a compound of the formula (16). A compound of the formula (16) is reacted with an acid such as trifluoroacetic acid, hydrochloric acid, sulfuric acid, and the like, in a solvent such as methylene chloride, 1,2-dichloroethane, tetrahydrofuran, 1,4-dioxane, methanol, ethanol, N,N-dimethylformamide, dimethylsulfoxide, N,N-dimethylacetamide, and the like, optionally in the presence of methylphenyl ether, optionally with heating, optionally with microwave irradiation to provide a compound of the formula (17). A compound of the formula (17) is reacted with a compound of the formula (17a), a known compound or a compound prepared by known methods, in the presence of a base such as sodium carbonate, potassium carbonate, lithium carbonate, triethylamine, pyridine, and the like, in a solvent such as N,N-dimethylformamide, dimethylsulfoxide, N,N-dimethylacetamide, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (17b).

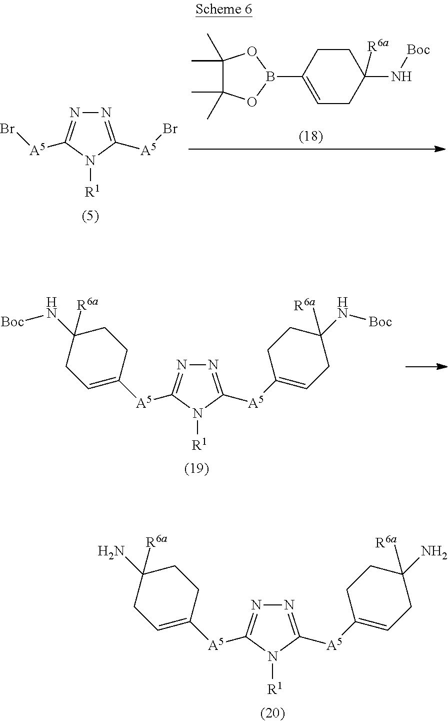

A compound of the formula (5) is reacted with a compound of the formula (18), a known compound or a compound prepared by known methods, in the presence of a palladium catalyst such as palladium (II) acetate, tetrakis(triphenylphosphine)palladium(0), dichlorobis (triphenylphosphine) palladium(II), palladium on carbon, bis(acetonitrile)dichloropalladium(II), and the like, in the presence of an organophosphine such as 2-dicyclohexylphosphino-2′,6′-dimethoxybiphenyl, 2-dicyclohexylphosphino-2′-(N,N-dimethylamino)biphenyl, 2-dicyclohexylphosphino-2′,4′,6′-triisopropylbiphenyl, 2-di-tert-butylphosphino-2′,4′,6′-triisopropylbiphenyl, (2-biphenyl)dicyclohexyl phosphine, (2-biphenyl)di-tert-butylphosphine, 2-dicyclohexylphosphino-2′,6′-diisopropoxybiphenyl, 2-di-tert-butylphosphino-3,4,5,6-tetramethyl-2′,4′,6′-triisopropyl-1,1′-biphenyl. Sodium 2′-dicyclohexylphosphino-2,6-dimethoxy-1,1′-biphenyl-3-sulfonate, 2-di-tert-butylphosphino-2′-methyl biphenyl, 2-dicyclohexylphosphino-2′-methylbiphenyl, 2′-(di-tert-butylphosphino)-N,N-dimethyl biphenyl-2-amine, 2′-(diphenylphosphino)-N, N′-dimethyl-(1,1′-biphenyl)-2-amine, and the like, in the presence of a base such as sodium carbonate, lithium carbonate, potassium carbonate, cesium carbonate, sodium hydroxide, lithium hydroxide, potassium hydroxide, triethylamine, N,N-diisopropylethylamine, pyridine, 2,6-dimethylpyridine, and the like, in a solvent such as tetrahydrofuran, 1,4-dioxane, acetonitrile, methylene chloride, 1,2-dichloroethane, 1,2-dimethoxyethane, and the like, optionally with heating, optionally with microwave irradiation to provide a compound of the formula (19). A compound of the formula (19) is reacted with an acid such as trifluoroacetic acid, hydrochloric acid, sulfuric acid, and the like, in a solvent such as methylene chloride, 1,2-dichloroethane, tetrahydrofuran, 1,4-dioxane, methanol, ethanol, N,N-dimethylformamide, dimethylsulfoxide, N,N-dimethylacetamide, and the like, optionally in the presence of methylphenyl ether, optionally with heating, optionally with microwave irradiation to provide a compound of the formula (20).

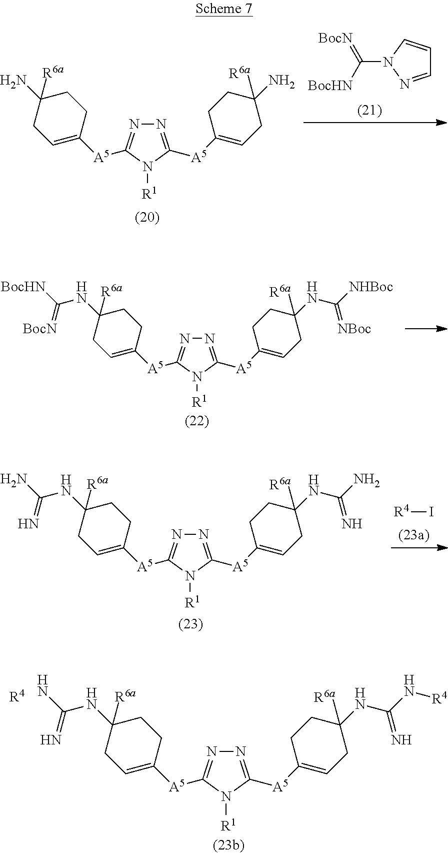

A compound of the formula (20) is reacted with a compound of the formula (21) in the presence of a base such as triethylamine, N,N-diisopropylethylamine, pyridine, 2,6-dimethylpyridine, and the like, in the presence of a solvent such as N,N-dimethylformamide, dimethylsulfoxide, N,N-dimethylacetamide, methylene chloride, 1,2-dichloroethane, tetrahydrofuran, 1,4-dioxane, acetonitrile, 1,2-dimethoxyethane, and the like, optionally with heating, optionally with microwave irradiation to provide a compound of the formula (22). A compound of the formula (22) is reacted with an acid such as trifluoroacetic acid, hydrochloric acid, sulfuric acid, and the like, in a solvent such as methylene chloride, 1,2-dichloroethane, tetrahydrofuran, 1,4-dioxane, methanol, ethanol, N,N-dimethylformamide, dimethylsulfoxide, N,N-dimethylacetamide, and the like, optionally in the presence of methylphenyl ether, optionally with heating, optionally with microwave irradiation to provide a compound of the formula (23). A compound of the formula (23) is reacted with a compound of the formula (23a), a known compound or a compound prepared by known methods, in the presence of a base such as sodium carbonate, potassium carbonate, lithium carbonate, triethylamine, pyridine, and the like, in a solvent such as N,N-dimethylformamide, dimethylsulfoxide, N,N-dimethylacetamide, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (23b).

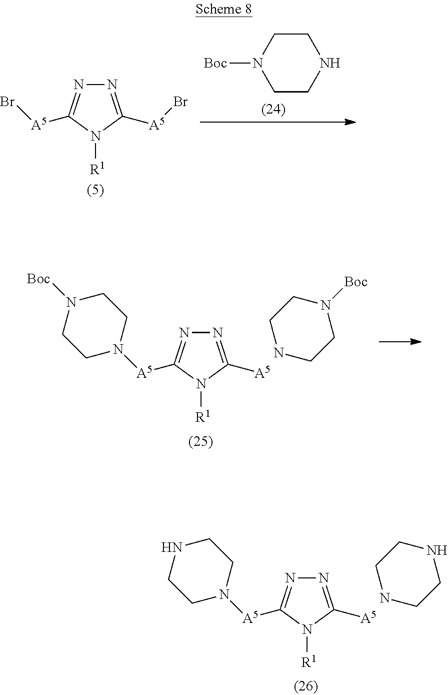

A compound of the formula (5) is reacted with a compound of the formula (24), a known compound or a compound prepared by known methods, in the presence of a palladium catalyst such as palladium (II) acetate, tetrakis(triphenylphosphine)palladium(0), dichlorobis (triphenylphosphine) palladium(II), palladium on carbon, bis(acetonitrile)dichloropalladium(II), and the like, in the presence of an organophosphine such as 2-dicyclohexylphosphino-2′,6′-dimethoxy biphenyl, 2-dicyclohexylphosphino-2′-(N,N-dimethylamino)biphenyl, 2-dicyclohexylphosphino-2′,4′,6′-triisopropyl biphenyl, 2-di-tert-butylphosphino-2′,4′,6′-triisopropylbiphenyl, (2-biphenyl)dicyclohexylphosphine, (2-biphenyl)di-tert-butylphosphine, 2-dicyclohexylphosphino-2′,6′-diisopropoxybiphenyl, 2-di-tert-butylphosphino-3,4,5,6-tetramethyl-2′,4′,6′-triisopropyl-1,1′-biphenyl, Sodium 2′-dicyclohexylphosphino-2,6-dimethoxy-1,1′-biphenyl-3-sulfonate, 2-di-tert-butylphosphino-2′-methyl biphenyl, 2-dicyclohexylphosphino-2′-methylbiphenyl, 2′-(di-tert-butylphosphino)-N,N-dimethyl biphenyl-2-amine, 2′-(diphenylphosphino)-N,N′-dimethyl-(1,1′-biphenyl)-2-amine, and the like, in the presence of a base such as sodium carbonate, lithium carbonate, potassium carbonate, cesium carbonate, sodium hydroxide, lithium hydroxide, potassium hydroxide, triethylamine, N,N-diisopropylethylamine, pyridine, 2,6-dimethylpyridine, and the like, in a solvent such as tetrahydrofuran, 1,4-dioxane, acetonitrile, methylene chloride, chloroform, 1,2-dichloroethane, 1,2-dimethoxyethane, and the like, optionally with heating, optionally with microwave irradiation to provide a compound of the formula (25). A compound of the formula (25) is reacted with an acid such as trifluoroacetic acid, hydrochloric acid, sulfuric acid, and the like, in a solvent such as methylene chloride, 1,2-dichloroethane, tetrahydrofuran, 1,4-dioxane, methanol, ethanol, N,N-dimethylformamide, dimethylsulfoxide, N,N-dimethylacetamide, and the like, optionally in the presence of methylphenyl ether, optionally with heating, optionally with microwave irradiation to provide a compound of the formula (26).

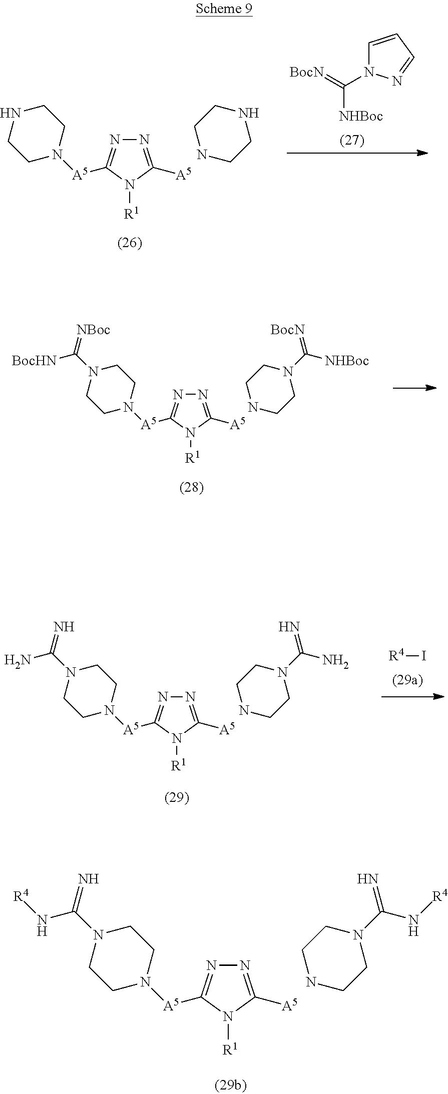

A compound of the formula (26) is reacted with a compound of the formula (27) in the presence of a base such as triethylamine, N,N-diisopropylethylamine, pyridine, 2,6-dimethylpyridine, and the like, in the presence of a solvent such as N,N-dimethylformamide, dimethylsulfoxide, N,N-dimethylacetamide, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (28). A compound of the formula (28) is reacted with an acid such as trifluoroacetic acid, hydrochloric acid, sulfuric acid, and the like, in a solvent such as methylene chloride, chloroform, 1,2-dichloroethane, tetrahydrofuran, 1,4-dioxane, methanol, ethanol, N,N-dimethylformamide, dimethylsulfoxide, N,N-dimethylacetamide, and the like, optionally in the presence of methylphenyl ether, optionally with heating, optionally with microwave irradiation to provide a compound of the formula (29). A compound of the formula (29) is reacted with a compound of the formula (29a), a known compound or a compound prepared by known methods, in the presence of a base such as sodium carbonate, potassium carbonate, lithium carbonate, triethylamine, pyridine, and the like, in a solvent such as N,N-dimethylformamide, dimethylsulfoxide, N,N-dimethylacetamide, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (29b).

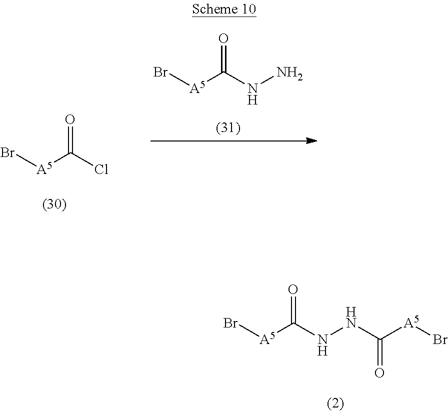

A compound of the formula (30), a known compound or a compound prepared by known methods, is reacted with a compound of the formula (31), a known compound or a compound prepared by known methods, in the presence of a base such as triethylamine, N,N-diisopropylethylamine, pyridine, 2,6-dimethylpyridine, and the like, in the presence of a solvent such as N,N-dimethylformamide, dimethylsulfoxide, N,N-dimethylacetamide, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (2).

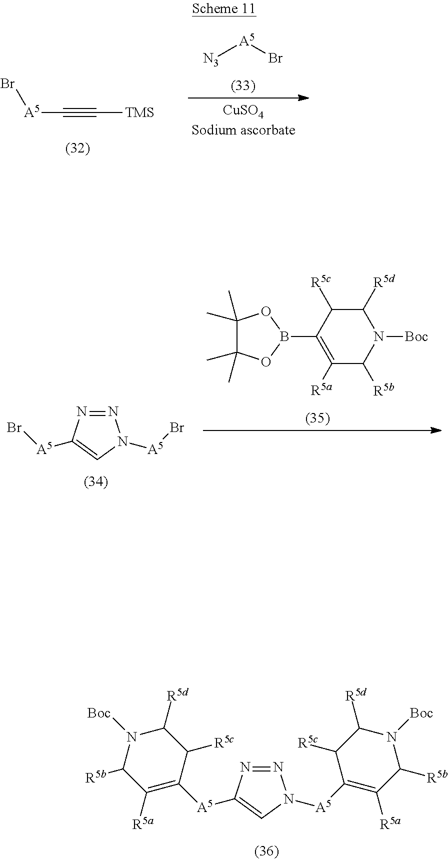

A compound of the formula (32), a known compound or a compound prepared by known methods, is reacted with a compound of the formula (33), a known compound or a compound prepared by known methods, in the presence of copper sulfate, in the presence of sodium ascorbate, optionally in the presence of potassium fluoride, in a solvent such as tetrahydrofuran, 1,4-dioxane acetonitrile, 1,2-dimethoxyethane, methanol, ethanol, and the like, optionally in the presence of water, optionally with heating; optionally with microwave irradiation to provide a compound of the formula (34). A compound of the formula (34) is reacted with a compound of the formula (35), a known compound or a compound prepared by known methods, in the presence of a palladium catalyst such as palladium (II) acetate, tetrakis(triphenylphosphine)palladium(0); dichlorobis (triphenylphosphine) palladium(II), palladium on carbon, bis(acetonitrile)dichloropalladium(II), and the like, in the presence of an organophosphine such as 2-dicyclohexylphosphino-2′,6′-dimethoxybiphenyl, 2-dicyclohexylphosphino-2′-(N,N-dimethylamino)biphenyl, 2-dicyclohexylphosphino-2′,4′,6′-triisopropylbiphenyl, 2-di-tert-butylphosphino-2′,4′,6′-triisopropylbiphenyl, (2-biphenyl)dicyclohexyl phosphine, (2-biphenyl)di-tert-butylphosphine, 2-dicyclohexylphosphino-2′,6′-diisopropoxybiphenyl, 2-di-tert-butylphosphino-3,4,5,6-tetramethyl-2′,4′,6′-triisopropyl-1,1′-biphenyl, Sodium 2′-dicyclohexylphosphino-2,6-dimethoxy-1,1′-biphenyl-3-sulfonate, 2-di-tert-butylphosphino-2′-methyl biphenyl, 2-dicyclohexylphosphino-2′-methylbiphenyl, 2′-(di-tert-butylphosphino)-N,N-dimethyl biphenyl-2-amine, 2′-(diphenylphosphino)-N,N′-dimethyl-(1,1′-biphenyl)-2-amine, and the like, in the presence of a base such as sodium carbonate, lithium carbonate, potassium carbonate, cesium carbonate, sodium hydroxide, lithium hydroxide, potassium hydroxide, triethylamine, N,N-diisopropylethylamine, pyridine, 2,6-dimethylpyridine, and the like, in a solvent such as tetrahydrofuran, 1,4-dioxane, acetonitrile, methylene chloride, chloroform, 1,2-dichloroethane, 1,2-dimethoxyethane, and the like, optionally with heating, optionally with microwave irradiation to provide a compound of the formula (36).

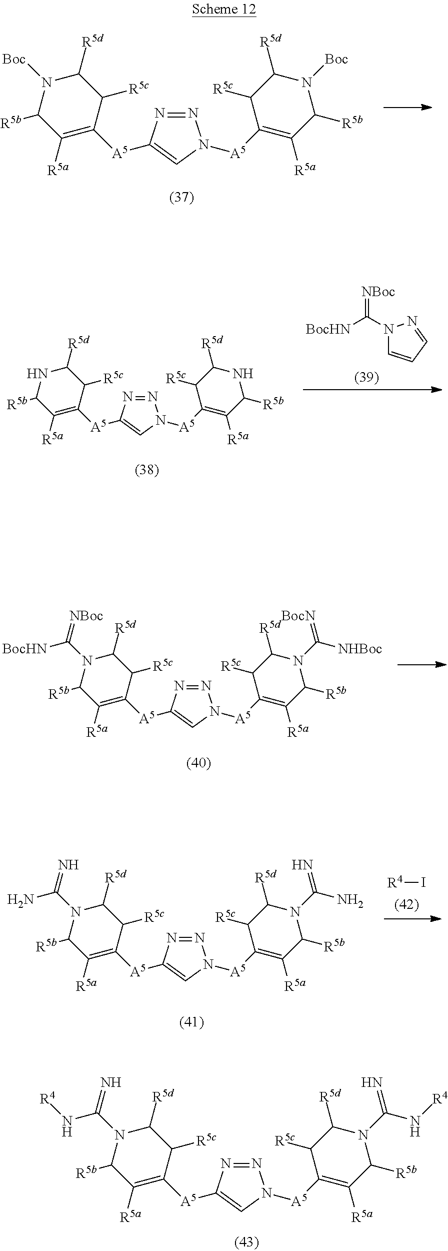

A compound of the formula (37) is reacted with an acid such as trifluoroacetic acid, hydrochloric acid, sulfuric acid, and the like, in a solvent such as methylene chloride, 1,2-dichloroethane, tetrahydrofuran, 1,4-dioxane, methanol, ethanol, N,N-dimethylformamide, dimethylsulfoxide, N,N-dimethylacetamide, and the like, optionally in the presence of methylphenyl ether, optionally with heating, optionally with microwave irradiation to provide a compound of the formula (38). A compound of the formula (38) is reacted with a compound of the formula (39) in the presence of a base such as triethylamine, N,N-diisopropylethylamine, pyridine, 2,6-dimethylpyridine, and the like, in the presence of a solvent such as N,N-dimethylformamide, dimethylsulfoxide, N,N-dimethylacetamide, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (40). A compound of the formula (40) is reacted with an acid such as trifluoroacetic acid, hydrochloric acid, sulfuric acid, and the like, in a solvent such as methylene chloride, chloroform, 1,2-dichloroethane, tetrahydrofuran, 1,4-dioxane, methanol, ethanol, N,N-dimethylformamide, dimethylsulfoxide, N,N-dimethylacetamide, and the like, optionally in the presence of methylphenyl ether, optionally with heating, optionally with microwave irradiation to provide a compound of the formula (41). A compound of the formula (41) is reacted with a compound of the formula (42), a known compound or a compound prepared by known methods, in the presence of a base such as sodium carbonate, potassium carbonate, lithium carbonate, triethylamine, pyridine, and the like, in a solvent such as N,N-dimethylformamide, dimethylsulfoxide, N,N-dimethylacetamide, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (43).

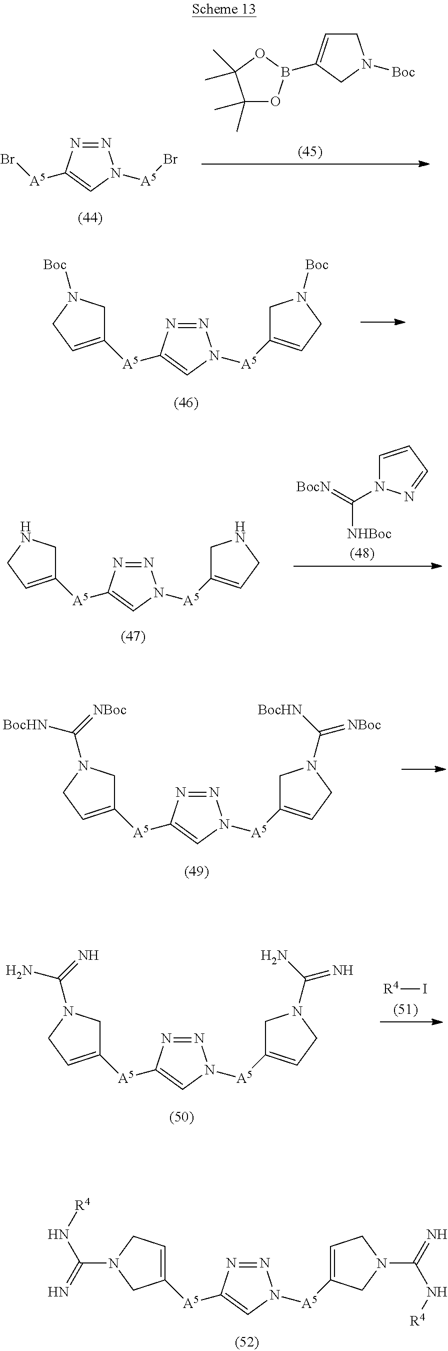

A compound of the formula (44) is reacted with a compound of the formula (45), a known compound or a compound prepared by known methods, in the presence of a palladium catalyst such as palladium (II) acetate, tetrakis(triphenylphosphine)palladium(0), dichlorobis (triphenylphosphine) palladium(II), palladium on carbon, bis(acetonitrile)dichloropalladium(II), and the like, in the presence of an organophosphine such as 2-dicyclohexylphosphino-2′,6′-dimethoxybiphenyl, 2-dicyclohexylphosphino-2′-(N,N-dimethylamino)biphenyl, 2-dicyclohexylphosphino-2′,4′,6′-triisopropylbiphenyl, 2-di-tert-butylphosphino-2′,4′,6′-triisopropylbiphenyl, (2-biphenyl)dicyclohexyl phosphine, (2-biphenyl)di-tert-butylphosphine, 2-dicyclohexylphosphino-2′,6′-diisopropoxybiphenyl, 2-di-tert-butylphosphino-3,4,5,6-tetramethyl-2′,4′,6′-triisopropyl-1,1′-biphenyl, Sodium 2′-dicyclohexylphosphino-2,6-dimethoxy-1,1′-biphenyl-3-sulfonate, 2-di-tert-butylphosphino-2′-methyl biphenyl, 2-dicyclohexylphosphino-2′-methylbiphenyl, 2′-(di-tert-butylphosphino)-N,N-dimethyl biphenyl-2-amine, 2′-(diphenylphosphino)-N,N′-dimethyl-(1,1′-biphenyl)-2-amine, and the like, in the presence of a base such as sodium carbonate, lithium carbonate, potassium carbonate, cesium carbonate, sodium hydroxide, lithium hydroxide, potassium hydroxide, triethylamine, N,N-diisopropylethylamine, pyridine, 2,6-dimethylpyridine, and the like, in a solvent such as tetrahydrofuran, 1,4-dioxane, acetonitrile, methylene chloride, chloroform, 1,2-dichloroethane, 1,2-dimethoxyethane, and the like, optionally with heating, optionally with microwave irradiation to provide a compound of the formula (46). A compound of the formula (46) is reacted with an acid such as trifluoroacetic acid, hydrochloric acid, sulfuric acid, and the like, in a solvent such as methylene chloride, 1,2-dichloroethane, tetrahydrofuran, 1,4-dioxane, methanol, ethanol, N,N-dimethylformamide, dimethylsulfoxide, N,N-dimethylacetamide, and the like, optionally in the presence of methylphenyl ether, optionally with heating, optionally with microwave irradiation to provide a compound of the formula (47). A compound of the formula (47) is reacted with a compound of the formula (48) in the presence of a base such as triethylamine, N,N-diisopropylethylamine, pyridine, 2,6-dimethylpyridine, and the like, in the presence of a solvent such as N,N-dimethylformamide, dimethylsulfoxide, N,N-dimethylacetamide, methylene chloride, 1,2-dichloroethane, tetrahydrofuran, 1,4-dioxane, acetonitrile, 1,2-dimethoxyethane, and the like, optionally with heating, optionally with microwave irradiation to provide a compound of the formula (49). A compound of the formula (49) is reacted with an acid such as trifluoroacetic acid, hydrochloric acid, sulfuric acid, and the like, in a solvent such as methylene chloride, 1,2-dichloroethane, tetrahydrofuran, 1,4-dioxane, methanol, ethanol, N,N-dimethylformamide, dimethylsulfoxide, N,N-dimethylacetamide, and the like, optionally in the presence of methylphenyl ether, optionally with heating, optionally with microwave irradiation to provide a compound of the formula (50). A compound of the formula (50) is reacted with a compound of the formula (51), a known compound or a compound prepared by known methods, in the presence of a base such as sodium carbonate, potassium carbonate, lithium carbonate, triethylamine, pyridine, and the like, in a solvent such as N,N-dimethylformamide, dimethylsulfoxide, N,N-dimethylacetamide, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (52).

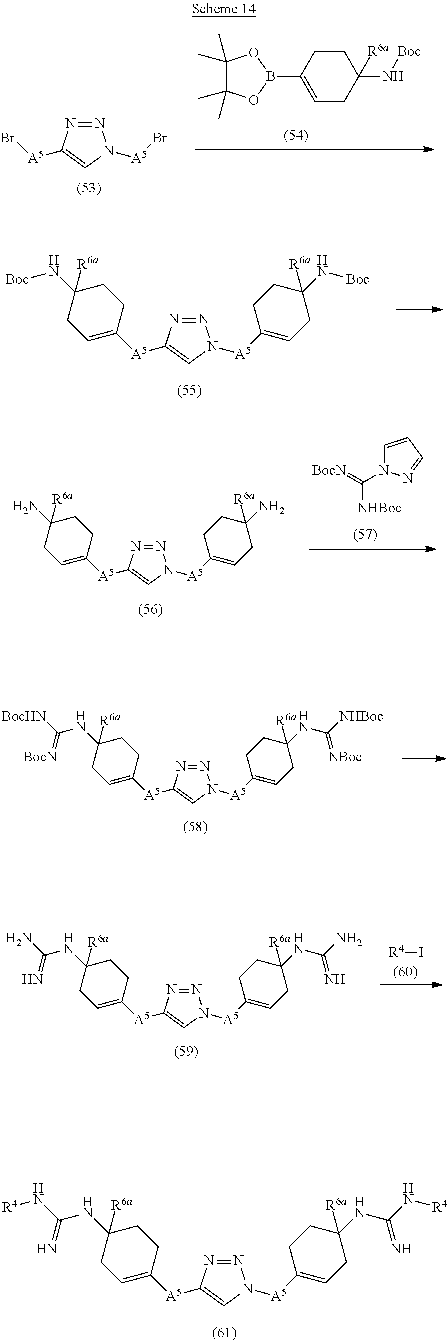

A compound of the formula (53) is reacted with a compound of the formula (54), a known compound or a compound prepared by known methods, in the presence of a palladium catalyst such as palladium (II) acetate, tetrakis(triphenylphosphine)palladium(0), dichlorobis (triphenylphosphine) palladium(II), palladium on carbon, bis(acetonitrile)dichloropalladium(II), and the like, in the presence of an organophosphine such as 2-dicyclohexylphosphino-2′,6′-dimethoxybiphenyl, 2-dicyclohexylphosphino-2′-(N,N-dimethylamino)biphenyl, 2-dicyclohexylphosphino-2′,4′,6′-triisopropylbiphenyl, 2-di-tert-butylphosphino-2′,4′,6′-triisopropylbiphenyl, (2-biphenyl)dicyclohexyl phosphine, (2-biphenyl)di-tert-butylphosphine, 2-dicyclohexylphosphino-2′,6′-diisopropoxybiphenyl, 2-di-tert-butylphosphino-3,4,5,6-tetramethyl-2′,4′,6′-triisopropyl-1,1′-biphenyl. Sodium 2′-dicyclohexylphosphino-2,6-dimethoxy-1,1′-biphenyl-3-sulfonate, 2-di-tert-butylphosphino-2′-methyl biphenyl, 2-dicyclohexylphosphino-2′-methylbiphenyl, 2′-(di-tert-butylphosphino)-N,N-dimethyl biphenyl-2-amine, 2′-(diphenylphosphino)-N,N′-dimethyl-(1,1′-biphenyl)-2-amine, and the like, in the presence of a base such as sodium carbonate, lithium carbonate, potassium carbonate, cesium carbonate, sodium hydroxide, lithium hydroxide, potassium hydroxide, triethylamine, N,N-diisopropylethylamine, pyridine, 2,6-dimethylpyridine, and the like, in a solvent such as tetrahydrofuran, 1,4-dioxane, acetonitrile, methylene chloride, chloroform, 1,2-dichloroethane, 1,2-dimethoxyethane, and the like, optionally with heating, optionally with microwave irradiation to provide a compound of the formula (55). A compound of the formula (55) is reacted with an acid such as trifluoroacetic acid, hydrochloric acid, sulfuric acid, and the like, in a solvent such as methylene chloride, 1,2-dichloroethane, tetrahydrofuran, 1,4-dioxane, methanol, ethanol, N,N-dimethylformamide, dimethylsulfoxide, N,N-dimethylacetamide, and the like, optionally in the presence of methylphenyl ether, optionally with heating, optionally with microwave irradiation to provide a compound of the formula (56). A compound of the formula (56) is reacted with a compound of the formula (57) in the presence of a base such as triethylamine, N,N-diisopropylethylamine, pyridine, 2,6-dimethylpyridine, and the like, in the presence of a solvent such as N,N-dimethylformamide, dimethylsulfoxide, N,N-dimethylacetamide, methylene chloride, 1,2-dichloroethane, tetrahydrofuran, 1,4-dioxane, acetonitrile, 1,2-dimethoxyethane, and the like, optionally with heating, optionally with microwave irradiation to provide a compound of the formula (58). A compound of the formula (58) is reacted with an acid such as trifluoroacetic acid, hydrochloric acid, sulfuric acid, and the like, in a solvent such as methylene chloride, 1,2-dichloroethane, tetrahydrofuran, 1,4-dioxane, methanol, ethanol. N,N-dimethylformamide, dimethylsulfoxide, N,N-dimethylacetamide, and the like, optionally in the presence of methylphenyl ether, optionally with heating, optionally with microwave irradiation to provide a compound of the formula (59). A compound of the formula (59) is reacted with a compound of the formula (60), a known compound or a compound prepared by known methods, in the presence of a base such as sodium carbonate, potassium carbonate, lithium carbonate, triethylamine, pyridine, and the like, in a solvent such as N,N-dimethylformamide, dimethylsulfoxide, N,N-dimethylacetamide, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (61).

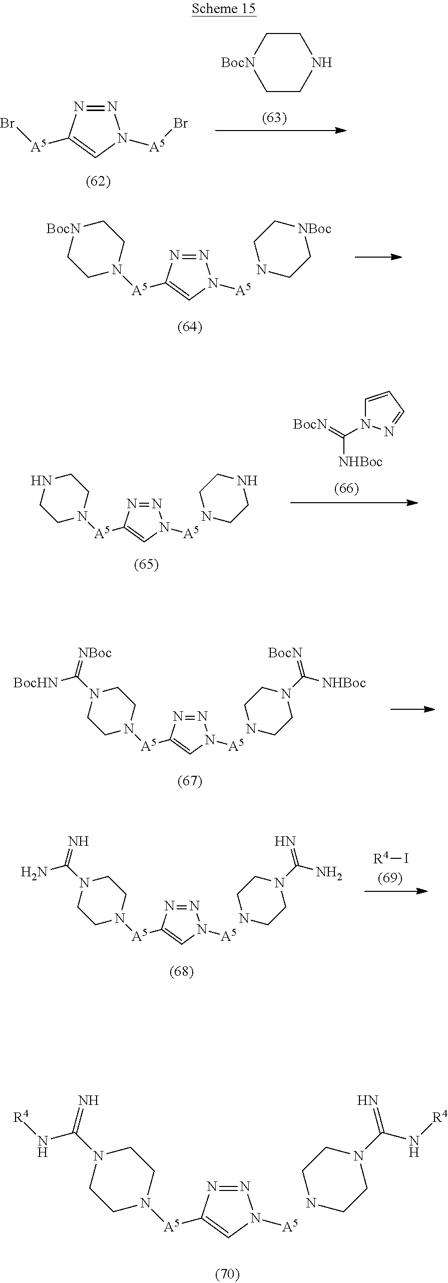

A compound of the formula (62) is reacted with a compound of the formula (63), a known compound or a compound prepared by known methods, in the presence of a palladium catalyst such as palladium (ii) acetate, tetrakis(triphenylphosphine)palladium(0), dichlorobis (triphenylphosphine) palladium(II), palladium on carbon, bis(acetonitrile)dichloropalladium(II), and the like, in the presence of an organophosphine such as 2-dicyclohexylphosphino-2′,6′-dimethoxybiphenyl, 2-dicyclohexylphosphino-2′-(N,N-dimethylamino)biphenyl, 2-dicyclohexylphosphino-2′,4′,6′-triisopropylbiphenyl, 2-di-tert-butylphosphino-2′,4′,6′-triisopropylbiphenyl, (2-biphenyl)dicyclohexyl phosphine, (2-biphenyl)di-tert-butylphosphine, 2-dicyclohexylphosphino-2′,6′-diisopropoxybiphenyl, 2-di-tert-butylphosphino-3,4,5,6-tetramethyl-2′,4′,6′-triisopropyl-1,1′-biphenyl, Sodium 2′-dicyclohexylphosphino-2,6-dimethoxy-1,1′-biphenyl-3-sulfonate, 2-di-tert-butylphosphino-2′-methyl biphenyl, 2-dicyclohexylphosphino-2′-methylbiphenyl, 2′-(di-tert-butylphosphino)-N,N-dimethyl biphenyl-2-amine, 2′-(diphenylphosphino)-N,N′-dimethyl-(1,1′-biphenyl)-2-amine, and the like, in the presence of a base such as sodium carbonate, lithium carbonate, potassium carbonate, cesium carbonate, sodium hydroxide, lithium hydroxide, potassium hydroxide, triethylamine, N,N-diisopropylethylamine, pyridine, 2,6-dimethylpyridine, and the like, in a solvent such as tetrahydrofuran, 1,4-dioxane, acetonitrile, methylene chloride, chloroform, 1,2-dichloroethane, 1,2-dimethoxyethane, and the like, optionally with heating, optionally with microwave irradiation to provide a compound of the formula (64). A compound of the formula (64) is reacted with an acid such as trifluoroacetic acid, hydrochloric acid, sulfuric acid, and the like, in a solvent such as methylene chloride, 1,2-dichloroethane, tetrahydrofuran, 1,4-dioxane, methanol, ethanol, N,N-dimethylformamide, dimethylsulfoxide, N,N-dimethylacetamide, and the like, optionally in the presence of methylphenyl ether, optionally with heating, optionally with microwave irradiation to provide a compound of the formula (65). A compound of the formula (65) is reacted with a compound of the formula (66) in the presence of a base such as triethylamine, N,N-diisopropylethylamine, pyridine, 2,6-dimethylpyridine, and the like, in the presence of a solvent such as N,N-dimethylformamide, dimethylsulfoxide, N,N-dimethylacetamide, methylene chloride, 1,2-dichloroethane, tetrahydrofuran, 1,4-dioxane, acetonitrile, 1,2-dimethoxyethane, and the like, optionally with heating, optionally with microwave irradiation to provide a compound of the formula (67). A compound of the formula (67) is reacted with an acid such as trifluoroacetic acid, hydrochloric acid, sulfuric acid, and the like, in a solvent such as methylene chloride, 1,2-dichloroethane, tetrahydrofuran, 1,4-dioxane, methanol, ethanol, N,N-dimethylformamide, dimethylsulfoxide, N,N-dimethylacetamide, and the like, optionally in the presence of methylphenyl ether, optionally with heating, optionally with microwave irradiation to provide a compound of the formula (68). A compound of the formula (68) is reacted with a compound of the formula (69), a known compound or a compound prepared by known methods, in the presence of a base such as sodium carbonate, potassium carbonate, lithium carbonate, triethylamine, pyridine, and the like, in a solvent such as N,N-dimethylformamide, dimethylsulfoxide, N,N-dimethylacetamide, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (70).

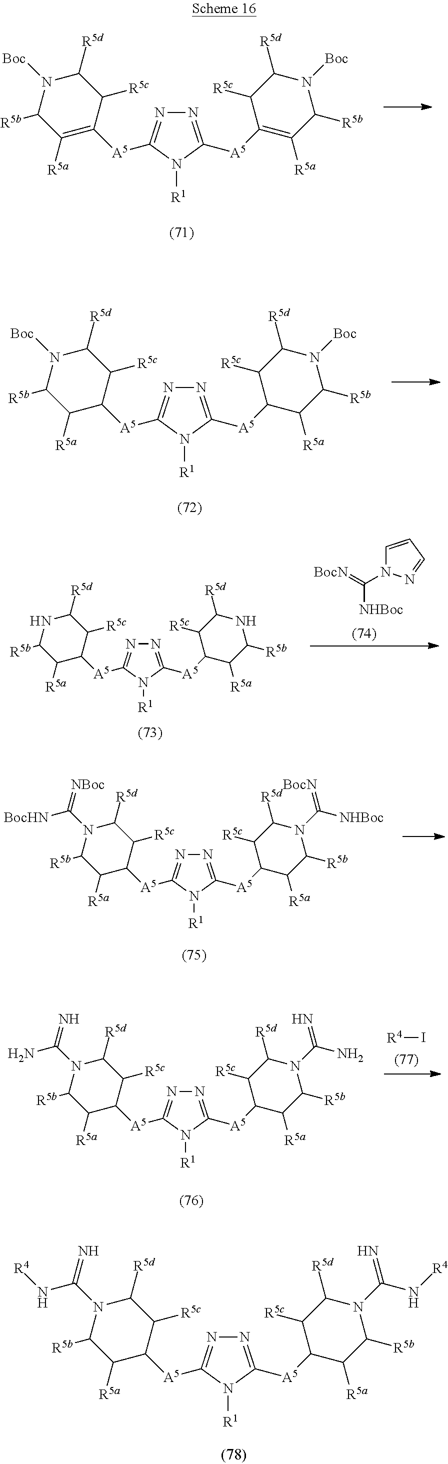

A compound of the formula (71) is reacted with hydrogen in the presence of a palladium catalyst such as palladium on carbon, palladium on barium sulfate, palladium on celite, palladium on silica gel, and the like, in the presence of a solvent such as methanol, ethanol, isopropanol, tetrahydrofuran, 1,4-dioxane, 1,2-dimethoxyethane, and the like, optionally with heating, optionally with microwave irradiation to provide a compound of the formula (72). A compound of the formula (72) is reacted with an acid such as trifluoroacetic acid, hydrochloric acid, sulfuric acid, and the like, in a solvent such as methylene chloride, 1,2-dichloroethane, tetrahydrofuran, 1,4-dioxane, methanol, ethanol, N,N-dimethylformamide, dimethylsulfoxide, N,N-dimethylacetamide, and the like, optionally in the presence of methylphenyl ether, optionally with heating, optionally with microwave irradiation to provide a compound of the formula (73). A compound of the formula (73) is reacted with a compound of the formula (74) in the presence of a base such as triethylamine, N,N-diisopropylethylamine, pyridine, 2,6-dimethylpyridine, and the like, in the presence of a solvent such as N,N-dimethylformamide, dimethylsulfoxide, N,N-dimethylacetamide, methylene chloride, 1,2-dichloroethane, tetrahydrofuran, 1,4-dioxane, acetonitrile, 1,2-dimethoxyethane, and the like, optionally with heating, optionally with microwave irradiation to provide a compound of the formula (75). A compound of the formula (75) is reacted with an acid such as trifluoroacetic acid, hydrochloric acid, sulfuric acid, and the like, in a solvent such as methylene chloride, 1,2-dichloroethane, tetrahydrofuran, 1,4-dioxane, methanol, ethanol. N,N-dimethylformamide, dimethylsulfoxide, N,N-dimethylacetamide, and the like, optionally in the presence of methylphenyl ether, optionally with heating, optionally with microwave irradiation to provide a compound of the formula (76). A compound of the formula (76) is reacted with a compound of the formula (77), a known compound or a compound prepared by known methods, in the presence of a base such as sodium carbonate, potassium carbonate, lithium carbonate, triethylamine, pyridine, and the like, in a solvent such as N,N-dimethylformamide, dimethylsulfoxide, N,N-dimethylacetamide, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (78).

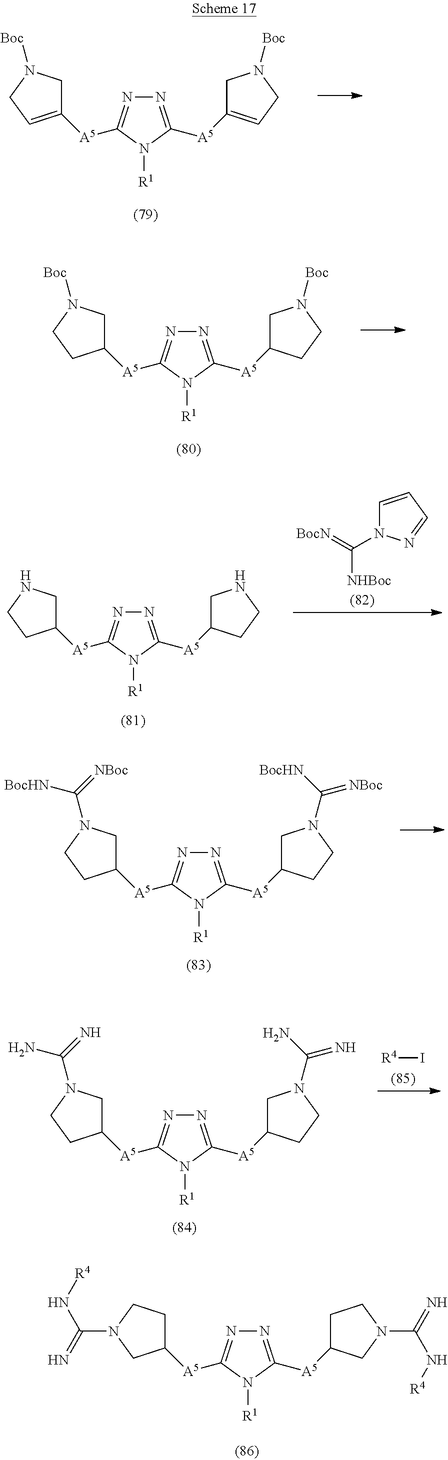

A compound of the formula (79) is reacted with hydrogen in the presence of a palladium catalyst such as palladium on carbon, palladium on barium sulfate, palladium on celite, palladium on silica gel, and the like, in the presence of a solvent such as methanol, ethanol, isopropanol, tetrahydrofuran, 1,4-dioxane, 1,2-dimethoxyethane, and the like, optionally with heating, optionally with microwave irradiation to provide a compound of the formula (80). A compound of the formula (80) is reacted with an acid such as trifluoroacetic acid, hydrochloric acid, sulfuric acid, and the like, in a solvent such as methylene chloride, 1,2-dichloroethane, tetrahydrofuran, 1,4-dioxane, methanol, ethanol, N,N-dimethylformamide, dimethylsulfoxide, N,N-dimethylacetamide, and the like, optionally in the presence of methylphenyl ether, optionally with heating, optionally with microwave irradiation to provide a compound of the formula (81). A compound of the formula (81) is reacted with a compound of the formula (82) in the presence of a base such as triethylamine, N,N-diisopropylethylamine, pyridine, 2,6-dimethylpyridine, and the like, in the presence of a solvent such as N,N-dimethylformamide, dimethylsulfoxide, N,N-dimethylacetamide, methylene chloride, 1,2-dichloroethane, tetrahydrofuran, 1,4-dioxane, acetonitrile, 1,2-dimethoxyethane, and the like, optionally with heating, optionally with microwave irradiation to provide a compound of the formula (83). A compound of the formula (83) is reacted with an acid such as trifluoroacetic acid, hydrochloric acid, sulfuric acid, and the like, in a solvent such as methylene chloride, 1,2-dichloroethane, tetrahydrofuran, 1,4-dioxane, methanol, ethanol, N,N-dimethylformamide, dimethylsulfoxide, N,N-dimethylacetamide, and the like, optionally in the presence of methylphenyl ether, optionally with heating, optionally with microwave irradiation to provide a compound of the formula (84). A compound of the formula (84) is reacted with a compound of the formula (85), a known compound or a compound prepared by known methods, in the presence of a base such as sodium carbonate, potassium carbonate, lithium carbonate, triethylamine, pyridine, and the like, in a solvent such as N,N-dimethylformamide, dimethylsulfoxide, N,N-dimethylacetamide, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (86).

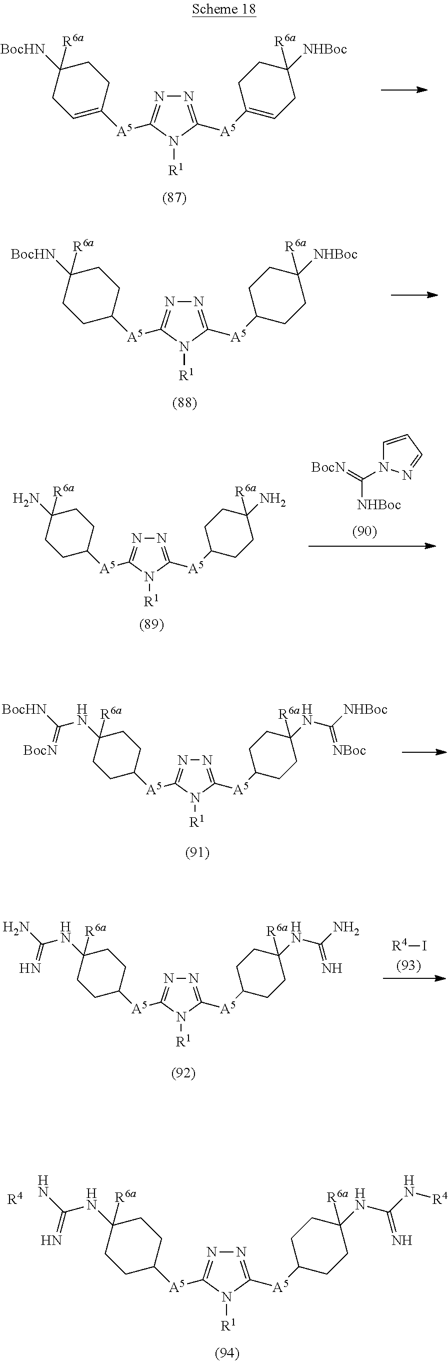

A compound of the formula (87) is reacted with hydrogen in the presence of a palladium catalyst such as palladium on carbon, palladium on barium sulfate, palladium on celite, palladium on silica gel, and the like, in the presence of a solvent such as methanol, ethanol, isopropanol, tetrahydrofuran, 1,4-dioxane, 1,2-dimethoxyethane, and the like, optionally with heating, optionally with microwave irradiation to provide a compound of the formula (88). A compound of the formula (88) is reacted with an acid such as trifluoroacetic acid, hydrochloric acid, sulfuric acid, and the like, in a solvent such as methylene chloride, 1,2-dichloroethane, tetrahydrofuran, 1,4-dioxane, methanol, ethanol, N,N-dimethylformamide, dimethylsulfoxide, N,N-dimethylacetamide, and the like, optionally in the presence of methylphenyl ether, optionally with heating, optionally with microwave irradiation to provide a compound of the formula (89). A compound of the formula (89) is reacted with a compound of the formula (90) in the presence of a base such as triethylamine, N,N-diisopropylethylamine, pyridine, 2,6-dimethylpyridine, and the like, in the presence of a solvent such as N,N-dimethylformamide, dimethylsulfoxide, N,N-dimethylacetamide, methylene chloride, 1,2-dichloroethane, tetrahydrofuran, 1,4-dioxane, acetonitrile, 1,2-dimethoxyethane, and the like, optionally with heating, optionally with microwave irradiation to provide a compound of the formula (91). A compound of the formula (91) is reacted with an acid such as trifluoroacetic acid, hydrochloric acid, sulfuric acid, and the like, in a solvent such as methylene chloride, 1,2-dichloroethane, tetrahydrofuran, 1,4-dioxane, methanol, ethanol, N,N-dimethylformamide, dimethylsulfoxide, N,N-dimethylacetamide, and the like, optionally in the presence of methylphenyl ether, optionally with heating, optionally with microwave irradiation to provide a compound of the formula (92). A compound of the formula (92) is reacted with a compound of the formula (93), a known compound or a compound prepared by known methods, in the presence of a base such as sodium carbonate, potassium carbonate, lithium carbonate, triethylamine, pyridine, and the like, in a solvent such as N,N-dimethylformamide, dimethylsulfoxide, N,N-dimethylacetamide, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (94).

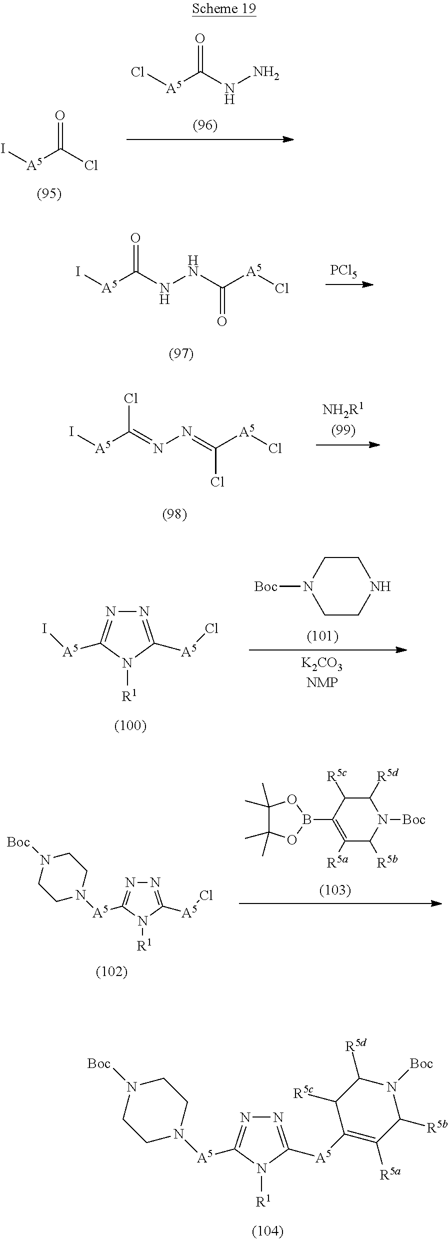

A compound of the formula (95), a known compound or a compound prepared by known methods, is reacted with a compound of the formula (96), a known compound or a compound prepared by known methods, in the presence of a base such as triethylamine, N,N-diisopropylethylamine, pyridine, 2,6-dimethylpyridine, and the like, in the presence of a solvent such as N,N-dimethylformamide, dimethylsulfoxide, N,N-dimethylacetamide, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (97). A compound of the formula (97) is reacted with phosphorous pentachloride in the presence of a solvent such as benzene, toluene, xylene, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (98). A compound of the formula (98) is reacted with a compound of the formula (99), a known compound or a compound prepared by known methods, in the presence of a solvent such as methanol, ethanol, isopropanol, methylene chloride, chloroform, 1,2-dichloroethane, tetrahydrofuran, 1,4-dioxane, acetonitrile, 1,2-dimethoxyethane, and the like, optionally in the presence of a base such as triethylamine, N,N-diisopropylethylamine, pyridine, 2,6-dimethylpyridine, and the like, optionally with heating, optionally with microwave irradiation to provide a compound of the formula (100). A compound of the formula (100) is reacted with a compound of the formula (101), a known compound or a compounds prepared by known methods, in the presence of a base such as sodium carbonate, lithium carbonate, potassium carbonate, cesium carbonate, sodium hydroxide, lithium hydroxide, potassium hydroxide, triethylamine, N,N-diisopropylethylamine, pyridine, 2,6-dimethylpyridine, and the like, in the presence of a solvent such as N-methyl-2-pyrrolidone, N,N-dimethylformamide, dimethylsulfoxide, N,N-dimethylacetamide, methylene chloride, chloroform, 1,2-dichloroethane, tetrahydrofuran, 1,4-dioxane, acetonitrile, 1,2-dimethoxyethane, and the like, optionally in the presence of methylphenyl ether, optionally with heating, optionally with microwave irradiation to provide a compound of the formula (102). A compound of the formula (102) is reacted with a compound of the formula (103), a known compound or a compound prepared by known methods, in the presence of a palladium catalyst such as palladium (II) acetate, tetrakis(triphenylphosphine)palladium(0), dichlorobis (triphenylphosphine) palladium(II), palladium on carbon, bis(acetonitrile)dichloropalladium(II), and the like, in the presence of an organophosphine such as 2-dicyclohexylphosphino-2′,6′-dimethoxybiphenyl, 2-dicyclohexylphosphino-2′-(N,N-dimethylamino)biphenyl, 2-dicyclohexylphosphino-2′,4′,6′-triisopropylbiphenyl, 2-di-tert-butylphosphino-2′,4′,6′-triisopropylbiphenyl, (2-biphenyl)dicyclohexyl phosphine, (2-biphenyl)di-tert-butylphosphine, 2-dicyclohexylphosphino-2′,6′-diisopropoxybiphenyl, 2-di-tert-butylphosphino-3,4,5,6-tetramethyl-2′,4′,6′-triisopropyl-1,1′-biphenyl, Sodium 2′-dicyclohexylphosphino-2,6-dimethoxy-1,1′-biphenyl-3-sulfonate, 2-di-tert-butylphosphino-2′-methyl biphenyl, 2-dicyclohexylphosphino-2′-methylbiphenyl, 2′-(di-tert-butylphosphino)-N,N-dimethyl biphenyl-2-amine, 2′-(diphenylphosphino)-N,N′-dimethyl-(1,1′-biphenyl)-2-amine, and the like, in the presence of a base such as sodium carbonate, lithium carbonate, potassium carbonate, cesium carbonate, sodium hydroxide, lithium hydroxide, potassium hydroxide, triethylamine, N,N-diisopropylethylamine, pyridine, 2,6-dimethylpyridine, and the like, in a solvent such as tetrahydrofuran, 1,4-dioxane, acetonitrile, methylene chloride, chloroform, 1,2-dichloroethane, 1,2-dimethoxyethane, and the like, optionally with heating, optionally with microwave irradiation to provide a compound of the formula (104).

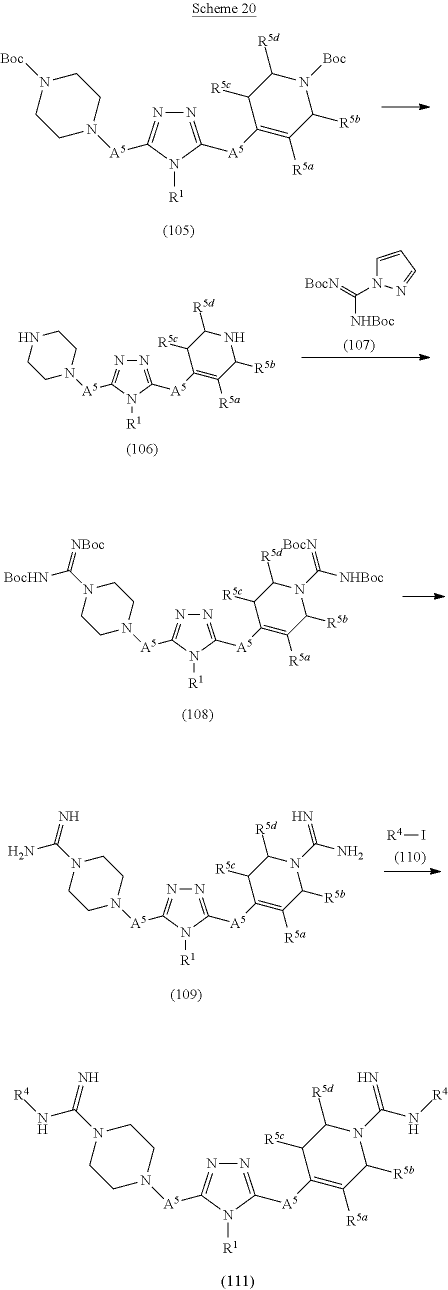

A compound of the formula (105) is reacted with an acid such as trifluoroacetic acid, hydrochloric acid, sulfuric acid, and the like, in a solvent such as methylene chloride, 1,2-dichloroethane, tetrahydrofuran, 1,4-dioxane, methanol, ethanol, N,N-dimethylformamide, dimethylsulfoxide, N,N-dimethylacetamide, and the like, optionally in the presence of methylphenyl ether, optionally with heating, optionally with microwave irradiation to provide a compound of the formula (106). A compound of the formula (106) is reacted with a compound of the formula (107) in the presence of a base such as triethylamine, N,N-diisopropylethylamine, pyridine, 2,6-dimethylpyridine, and the like, in the presence of a solvent such as N,N-dimethylformamide, dimethylsulfoxide, N,N-dimethylacetamide, methylene chloride, 1,2-dichloroethane, tetrahydrofuran, 1,4-dioxane, acetonitrile, 1,2-dimethoxyethane, and the like, optionally with heating, optionally with microwave irradiation to provide a compound of the formula (108). A compound of the formula (108) is reacted with an acid such as trifluoroacetic acid, hydrochloric acid, sulfuric acid, and the like, in a solvent such as methylene chloride, 1,2-dichloroethane, tetrahydrofuran, 1,4-dioxane, methanol, ethanol, N,N-dimethylformamide, dimethylsulfoxide, N,N-dimethylacetamide, and the like, optionally in the presence of methylphenyl ether, optionally with heating, optionally with microwave irradiation to provide a compound of the formula (109). A compound of the formula (109) is reacted with a compound of the formula (110), a known compound or a compound prepared by known methods, in the presence of a base such as sodium carbonate, potassium carbonate, lithium carbonate, triethylamine, pyridine, and the like, in a solvent such as N,N-dimethylformamide, dimethylsulfoxide, N,N-dimethylacetamide, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (111).

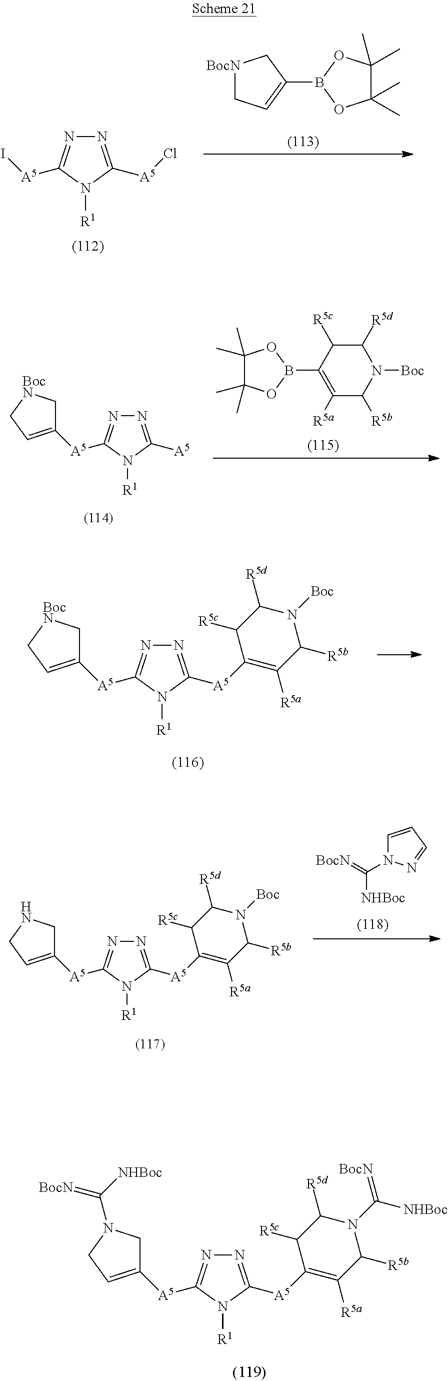

A compound of the formula (112) is reacted with a compound of the formula (113), a known compound or a compound prepared by known methods, in the presence of a palladium catalyst such as palladium (II) acetate, tetrakis(triphenylphosphine)palladium(0), dichlorobis (triphenylphosphine) palladium(II), palladium on carbon, bis(acetonitrile)dichloropalladium(II), and the like, in the presence of an organophosphine such as 2-dicyclohexylphosphino-2′,6′-dimethoxybiphenyl, 2-dicyclohexylphosphino-2′-(N,N-dimethylamino)biphenyl, 2-dicyclohexylphosphino-2′,4′,6′-triisopropylbiphenyl, 2-di-tert-butylphosphino-2′,4′,6′-triisopropylbiphenyl, (2-biphenyl)dicyclohexyl phosphine, (2-biphenyl)di-tert-butylphosphine, 2-dicyclohexylphosphino-2′,6′-diisopropoxybiphenyl, 2-di-tert-butylphosphino-3,4,5,6-tetramethyl-2′,4′,6′-triisopropyl-1,1′-biphenyl, Sodium 2′-dicyclohexylphosphino-2,6-dimethoxy-1,1′-biphenyl-3-sulfonate, 2-di-tert-butylphosphino-2′-methyl biphenyl, 2-dicyclohexylphosphino-2′-methylbiphenyl, 2′-(di-tert-butylphosphino)-N,N-dimethyl biphenyl-2-amine, 2′-(diphenylphosphino)-N,N′-dimethyl-(1,1′-biphenyl)-2-amine, and the like, in the presence of a base such as sodium carbonate, lithium carbonate, potassium carbonate, cesium carbonate, sodium hydroxide, lithium hydroxide, potassium hydroxide, triethylamine, N,N-diisopropylethylamine, pyridine, 2,6-dimethylpyridine, and the like, in a solvent such as tetrahydrofuran, 1,4-dioxane, acetonitrile, methylene chloride, chloroform, 1,2-dichloroethane, 1,2-dimethoxyethane, and the like, optionally with heating, optionally with microwave irradiation to provide a compound of the formula (114). A compound of the formula (114) is reacted with a compound of the formula (115), a known compound or a compound prepared by known methods, in the presence of a palladium catalyst such as palladium (II) acetate, tetrakis(triphenylphosphine)palladium(0), dichlorobis (triphenylphosphine) palladium(II), palladium on carbon, bis(acetonitrile)dichloropalladium(II), and the like, in the presence of an organophosphine such as 2-dicyclohexylphosphino-2′,6′-dimethoxybiphenyl, 2-dicyclohexylphosphino-2′-(N,N-dimethylamino)biphenyl, 2-dicyclohexylphosphino-2′,4′,6′-triisopropylbiphenyl, 2-di-tert-butylphosphino-2′,4′,6′-triisopropylbiphenyl, (2-biphenyl)dicyclohexyl phosphine, (2-biphenyl)di-tert-butylphosphine, 2-dicyclohexylphosphino-2′,6′-diisopropoxybiphenyl, 2-di-tert-butylphosphino-3,4,5,6-tetramethyl-2′,4′,6′-triisopropyl-1,1′-biphenyl, Sodium 2′-dicyclohexylphosphino-2,6-dimethoxy-1,1′-biphenyl-3-sulfonate, 2-di-tert-butylphosphino-2′-methyl biphenyl, 2-dicyclohexylphosphino-2′-methylbiphenyl, 2′-(di-tert-butylphosphino)-N,N-dimethyl biphenyl-2-amine, 2′-(diphenylphosphino)-N,N′-dimethyl-(1,1′-biphenyl)-2-amine, and the like, in the presence of a base such as sodium carbonate, lithium carbonate, potassium carbonate, cesium carbonate, sodium hydroxide, lithium hydroxide, potassium hydroxide, triethylamine, N,N-diisopropylethylamine, pyridine, 2,6-dimethylpyridine, and the like, in a solvent such as tetrahydrofuran, 1,4-dioxane, acetonitrile, methylene chloride, chloroform, 1,2-dichloroethane, 1,2-dimethoxyethane, and the like, optionally with heating, optionally with microwave irradiation to provide a compound of the formula (116). A compound of the formula (116) is reacted with an acid such as trifluoroacetic acid, hydrochloric acid, sulfuric acid, and the like, in a solvent such as methylene chloride, 1,2-dichloroethane, tetrahydrofuran, 1,4-dioxane, methanol, ethanol, N,N-dimethylformamide, dimethylsulfoxide, N,N-dimethylacetamide, and the like, optionally in the presence of methylphenyl ether, optionally with heating, optionally with microwave irradiation to provide a compound of the formula (117). A compound of the formula (117) is reacted with a compound of the formula (118) in the presence of a base such as triethylamine, N,N-diisopropylethylamine, pyridine, 2,6-dimethylpyridine, and the like, in the presence of a solvent such as N,N-dimethylformamide, dimethylsulfoxide, N,N-dimethylacetamide, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (119).

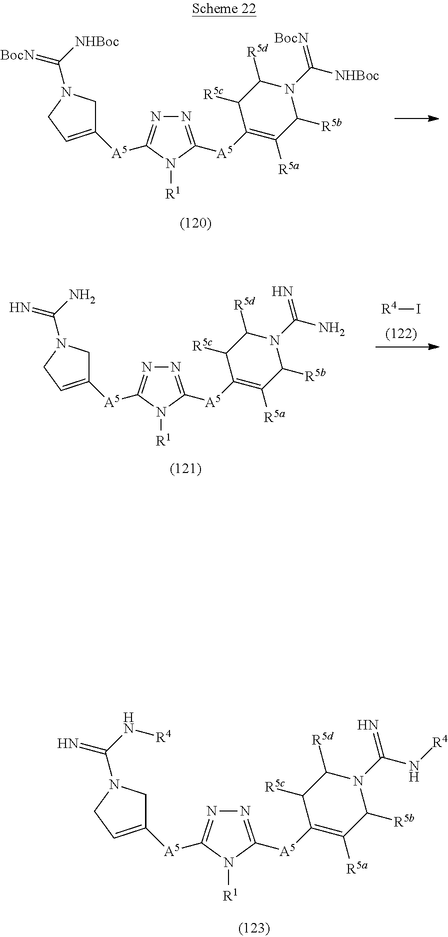

A compound of the formula (120) is reacted with an acid such as trifluoroacetic acid, hydrochloric acid, sulfuric acid, and the like, in a solvent such as methylene chloride, chloroform, 1,2-dichloroethane, tetrahydrofuran, 1,4-dioxane, methanol, ethanol, N,N-dimethylformamide, dimethylsulfoxide, N,N-dimethylacetamide, and the like, optionally in the presence of methylphenyl ether, optionally with heating, optionally with microwave irradiation to provide a compound of the formula (121). A compound of the formula (121) is reacted with a compound of the formula (122), a known compound or a compound prepared by known methods, in the presence of a base such as sodium carbonate, potassium carbonate, lithium carbonate, triethylamine, pyridine, and the like, in a solvent such as N,N-dimethylformamide, dimethylsulfoxide, N,N-dimethylacetamide, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (123).

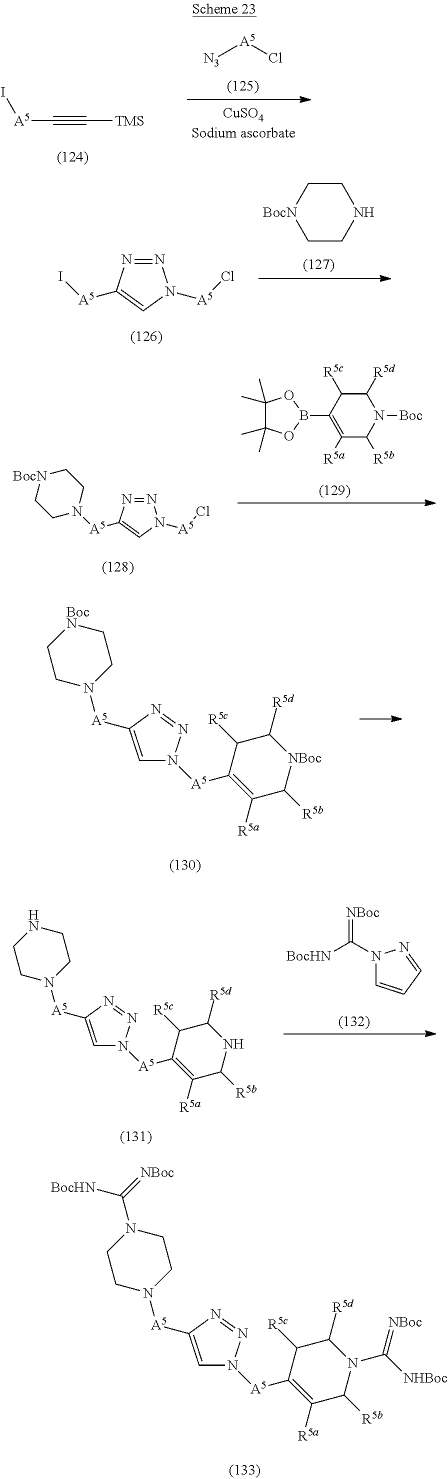

A compound of the formula (124), a known compound or a compound prepared by known methods, is reacted with a compound of the formula (125), a known compound or a compound prepared by known methods, in the presence of copper sulfate, in the presence of sodium ascorbate, optionally in the presence of potassium fluoride, in a solvent such as tetrahydrofuran, 1,4-dioxane, acetonitrile, 1,2-dimethoxyethane, methanol, ethanol, and the like, optionally in the presence of water, optionally with heating, optionally with microwave irradiation to provide a compound of the formula (126). A compound of the formula (126) is reacted with a compound of the formula (127), a known compound or a compounds prepared by known methods, in the presence of a base such as sodium carbonate, lithium carbonate, potassium carbonate, cesium carbonate, sodium hydroxide, lithium hydroxide, potassium hydroxide, triethylamine, N,N-diisopropylethylamine, pyridine, 2,6-dimethylpyridine, and the like, in the presence of a solvent such as N-methyl-2-pyrrolidone, N,N-dimethylformamide, dimethylsulfoxide, N,N-dimethylacetamide, methylene chloride, chloroform, 1,2-dichloroethane, tetrahydrofuran, 1,4-dioxane, acetonitrile, 1,2-dimethoxyethane, and the like, optionally in the presence of methylphenyl ether, optionally with heating, optionally with microwave irradiation to provide a compound of the formula (128). A compound of the formula (128) is reacted with a compound of the formula (129), a known compound or a compound prepared by known methods, in the presence of a palladium catalyst such as palladium (II) acetate, tetrakis(triphenylphosphine)palladium(0), dichlorobis (triphenylphosphine) palladium(II), palladium on carbon, bis(acetonitrile)dichloropalladium(II), and the like, in the presence of an organophosphine such as 2-dicyclohexylphosphino-2′,6′-dimethoxybiphenyl, 2-dicyclohexylphosphino-2′-(N,N-dimethylamino)biphenyl, 2-dicyclohexylphosphino-2′,4′,6′-triisopropylbiphenyl, 2-di-tert-butylphosphino-2′,4′,6′-triisopropylbiphenyl, (2-biphenyl)dicyclohexyl phosphine, (2-biphenyl)di-tert-butylphosphine, 2-dicyclohexylphosphino-2′,6′-diisopropoxybiphenyl, 2-di-tert-butylphosphino-3,4,5,6-tetramethyl-2′,4′,6′-triisopropyl-1,1′-biphenyl, Sodium 2′-dicyclohexylphosphino-2,6-dimethoxy-1,1′-biphenyl-3-sulfonate, 2-di-tert-butylphosphino-2′-methyl biphenyl, 2-dicyclohexylphosphino-2′-methylbiphenyl, 2′-(di-tert-butylphosphino)-N,N-dimethyl biphenyl-2-amine, 2′-(diphenylphosphino)-N,N′-dimethyl-(1,1′-biphenyl)-2-amine, and the like, in the presence of a base such as sodium carbonate, lithium carbonate, potassium carbonate, cesium carbonate, sodium hydroxide, lithium hydroxide, potassium hydroxide, triethylamine, N,N-diisopropylethylamine, pyridine, 2,6-dimethylpyridine, and the like, in a solvent such as tetrahydrofuran, 1,4-dioxane, acetonitrile, methylene chloride, chloroform, 1,2-dichloroethane, 1,2-dimethoxyethane, and the like, optionally with heating, optionally with microwave irradiation to provide a compound of the formula (130). A compound of the formula (130) is reacted with an acid such as trifluoroacetic acid, hydrochloric acid, sulfuric acid, and the like, in a solvent such as methylene chloride, 1,2-dichloroethane, tetrahydrofuran, 1,4-dioxane, methanol, ethanol, N,N-dimethylformamide, dimethylsulfoxide, N,N-dimethylacetamide, and the like, optionally in the presence of methylphenyl ether, optionally with heating, optionally with microwave irradiation to provide a compound of the formula (131). A compound of the formula (131) is reacted with a compound of the formula (132) in the presence of a base such as triethylamine, N,N-diisopropylethylamine, pyridine, 2,6-dimethylpyridine, and the like, in the presence of a solvent such as N,N-dimethylformamide, dimethylsulfoxide, N,N-dimethylacetamide, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (133).

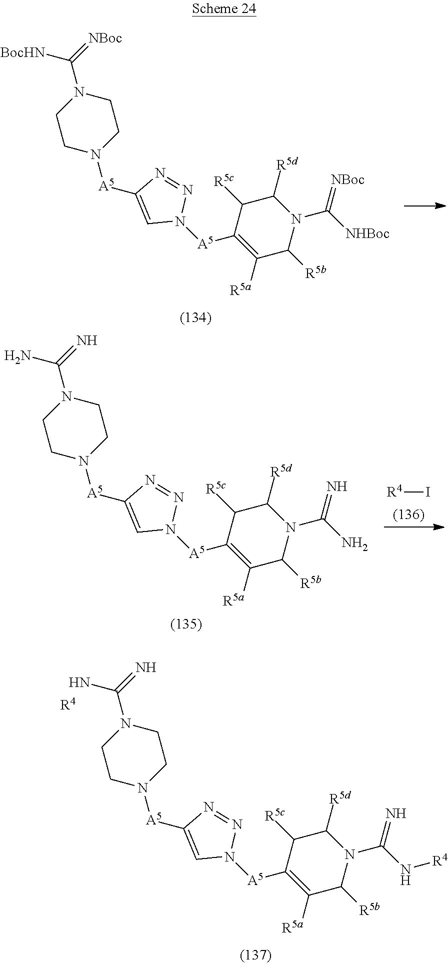

A compound of the formula (134) is reacted with an acid such as trifluoroacetic acid, hydrochloric acid, sulfuric acid, and the like, in a solvent such as methylene chloride, chloroform, 1,2-dichloroethane, tetrahydrofuran, 1,4-dioxane, methanol, ethanol, N,N-dimethylformamide, dimethylsulfoxide, N,N-dimethylacetamide, and the like, optionally in the presence of methylphenyl ether, optionally with heating, optionally with microwave irradiation to provide a compound of the formula (135). A compound of the formula (135) is reacted with a compound of the formula (136), a known compound or a compound prepared by known methods, in the presence of a base such as sodium carbonate, potassium carbonate, lithium carbonate, triethylamine, pyridine, and the like, in a solvent such as N,N-dimethylformamide, dimethylsulfoxide, N,N-dimethylacetamide, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (137).

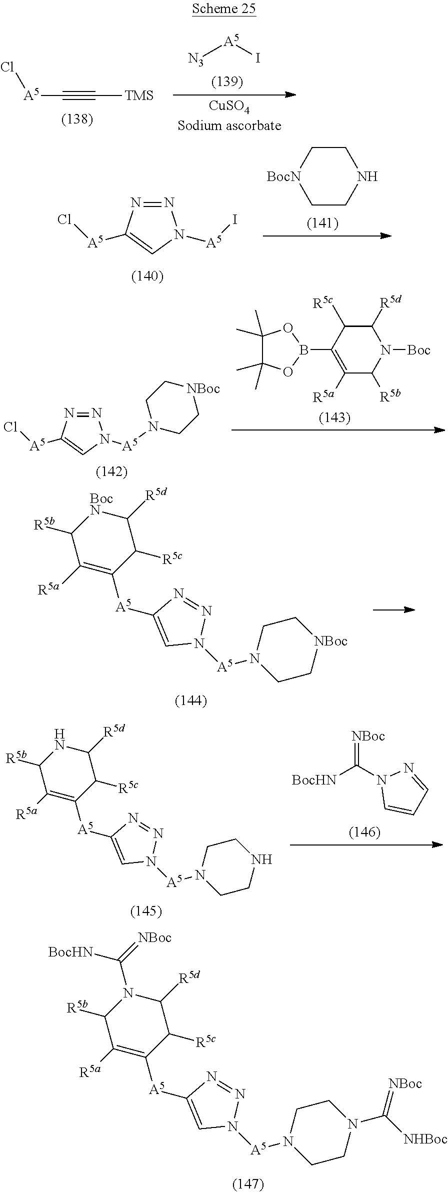

A compound of the formula (138), a known compound or a compound prepared by known methods, is reacted with a compound of the formula (139), a known compound or a compound prepared by known methods, in the presence of copper sulfate, in the presence of sodium ascorbate, optionally in the presence of potassium fluoride, in a solvent such as tetrahydrofuran, 1,4-dioxane, acetonitrile, 1,2-dimethoxyethane, methanol, ethanol, and the like, optionally in the presence of water, optionally with heating, optionally with microwave irradiation to provide a compound of the formula (140). A compound of the formula (140) is reacted with a compound of the formula (141), a known compound or a compounds prepared by known methods, in the presence of a base such as sodium carbonate, lithium carbonate, potassium carbonate, cesium carbonate, sodium hydroxide, lithium hydroxide, potassium hydroxide, triethylamine, N,N-diisopropylethylamine, pyridine, 2,6-dimethylpyridine, and the like, in the presence of a solvent such as N-methyl-2-pyrrolidone, N,N-dimethylformamide, dimethylsulfoxide, N,N-dimethylacetamide, methylene chloride, chloroform, 1,2-dichloroethane, tetrahydrofuran, 1,4-dioxane, acetonitrile, 1,2-dimethoxyethane, and the like, optionally in the presence of methylphenyl ether, optionally with heating, optionally with microwave irradiation to provide a compound of the formula (142). A compound of the formula (142) is reacted with a compound of the formula (143), a known compound or a compound prepared by known methods, in the presence of a palladium catalyst such as palladium (II) acetate, tetrakis(triphenylphosphine)palladium(0), dichlorobis (triphenylphosphine) palladium(II), palladium on carbon, bis(acetonitrile)dichloropalladium(II), and the like, in the presence of an organophosphine such as 2-dicyclohexylphosphino-2′,6′-dimethoxybiphenyl, 2-dicyclohexylphosphino-2′-(N,N-dimethylamino)biphenyl, 2-dicyclohexylphosphino-2′,4′,6′-triisopropylbiphenyl, 2-di-tert-butylphosphino-2′,4′,6′-triisopropylbiphenyl, (2-biphenyl)dicyclohexyl phosphine, (2-biphenyl)di-tert-butylphosphine, 2-dicyclohexylphosphino-2′,6′-diisopropoxybiphenyl, 2-di-tert-butylphosphino-3,4,5,6-tetramethyl-2′,4′,6′-tri isopropyl-1,1′-biphenyl, Sodium 2′-dicyclohexylphosphino-2,6-dimethoxy-1,1′-biphenyl-3-sulfonate, 2-di-tert-butylphosphino-2′-methyl biphenyl, 2-dicyclohexylphosphino-2′-methylbiphenyl, 2′-(di-tert-butylphosphino)-N,N-dimethyl biphenyl-2-amine, 2′-(diphenylphosphino)-N,N′-dimethyl-(1,1′-biphenyl)-2-amine, and the like, in the presence of a base such as sodium carbonate, lithium carbonate, potassium carbonate, cesium carbonate, sodium hydroxide, lithium hydroxide, potassium hydroxide, triethylamine, N,N-diisopropylethylamine, pyridine, 2,6-dimethylpyridine, and the like, in a solvent such as tetrahydrofuran, 1,4-dioxane, acetonitrile, methylene chloride, chloroform, 1,2-dichloroethane, 1,2-dimethoxyethane, and the like, optionally with heating, optionally with microwave irradiation to provide a compound of the formula (144). A compound of the formula (144) is reacted with an acid such as trifluoroacetic acid, hydrochloric acid, sulfuric acid, and the like, in a solvent such as methylene chloride, 1,2-dichloroethane, tetrahydrofuran, 1,4-dioxane, methanol, ethanol, N,N-dimethylformamide, dimethylsulfoxide, N,N-dimethylacetamide, and the like, optionally in the presence of methylphenyl ether, optionally with heating, optionally with microwave irradiation to provide a compound of the formula (145). A compound of the formula (145) is reacted with a compound of the formula (146) in the presence of a base such as triethylamine, N,N-diisopropylethylamine, pyridine, 2,6-dimethylpyridine, and the like, in the presence of a solvent such as N,N-dimethylformamide, dimethylsulfoxide, N,N-dimethylacetamide, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (147).

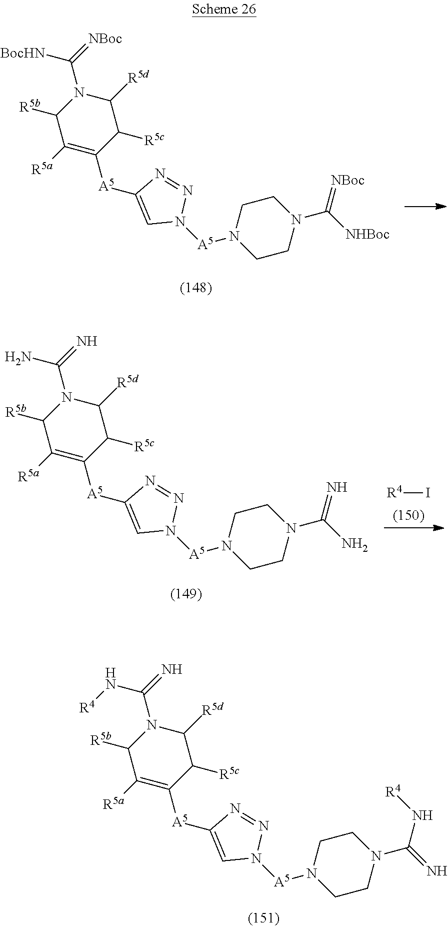

A compound of the formula (148) is reacted with an acid such as trifluoroacetic acid, hydrochloric acid, sulfuric acid, and the like, in a solvent such as methylene chloride, chloroform, 1,2-dichloroethane, tetrahydrofuran, 1,4-dioxane, methanol, ethanol, N,N-dimethylformamide, dimethylsulfoxide, N,N-dimethylacetamide, and the like, optionally in the presence of methylphenyl ether, optionally with heating, optionally with microwave irradiation to provide a compound of the formula (149). A compound of the formula (149) is reacted with a compound of the formula (150), a known compound or a compound prepared by known methods, in the presence of a base such as sodium carbonate, potassium carbonate, lithium carbonate, triethylamine, pyridine, and the like, in a solvent such as N,N-dimethylformamide, dimethylsulfoxide, N,N-dimethylacetamide, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (151).

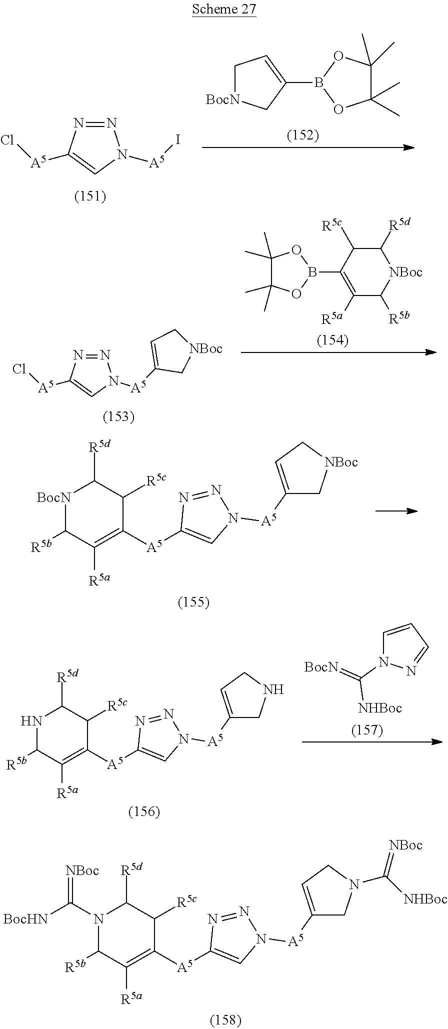

A compound of the formula (151) is reacted with a compound of the formula (152), a known compound or a compound prepared by known methods, in the presence of a palladium catalyst such as palladium (II) acetate, tetrakis(triphenylphosphine)palladium(0), dichlorobis (triphenylphosphine) palladium(II), palladium on carbon, bis(acetonitrile)dichloropalladium(II), and the like, in the presence of an organophosphine such as 2-dicyclohexylphosphino-2′,6′-dimethoxybiphenyl, 2-dicyclohexylphosphino-2′-(N,N-dimethylamino)biphenyl, 2-dicyclohexylphosphino-2′,4′,6′-triisopropylbiphenyl, 2-di-tert-butylphosphino-2′,4′,6′-triisopropylbiphenyl, (2-biphenyl)dicyclohexyl phosphine, (2-biphenyl)di-tert-butylphosphine, 2-dicyclohexylphosphino-2′,6′-diisopropoxybiphenyl, 2-di-tert-butylphosphino-3,4,5,6-tetramethyl-2′,4′,6′-triisopropyl-1,1′-biphenyl, Sodium 2′-dicyclohexylphosphino-2,6-dimethoxy-1,1′-biphenyl-3-sulfonate, 2-di-tert-butylphosphino-2′-methyl biphenyl, 2-dicyclohexylphosphino-2′-methylbiphenyl, 2′-(di-tert-butylphosphino)-N,N-dimethyl biphenyl-2-amine, 2′-(diphenylphosphino)-N,N′-dimethyl-(1,1′-biphenyl)-2-amine, and the like, in the presence of a base such as sodium carbonate, lithium carbonate, potassium carbonate, cesium carbonate, sodium hydroxide, lithium hydroxide, potassium hydroxide, triethylamine, N,N-diisopropylethylamine, pyridine, 2,6-dimethylpyridine, and the like, in a solvent such as tetrahydrofuran, 1,4-dioxane, acetonitrile, methylene chloride, chloroform, 1,2-dichloroethane, 1,2-dimethoxyethane, and the like, optionally with heating, optionally with microwave irradiation to provide a compound of the formula (153). A compound of the formula (153) is reacted with a compound of the formula (154), a known compound or a compound prepared by known methods, in the presence of a palladium catalyst such as palladium (II) acetate, tetrakis(triphenylphosphine)palladium(0), dichlorobis (triphenylphosphine) palladium(II), palladium on carbon, bis(acetonitrile)dichloropalladium(II), and the like, in the presence of an organophosphine such as 2-dicyclohexylphosphino-2′,6′-dimethoxybiphenyl, 2-dicyclohexylphosphino-2′-(N,N-dimethylamino)biphenyl, 2-dicyclohexylphosphino-2′,4′,6′-triisopropylbiphenyl, 2-di-tert-butylphosphino-2′,4′,6′-triisopropylbiphenyl, (2-biphenyl)dicyclohexyl phosphine, (2-biphenyl)di-tert-butylphosphine, 2-dicyclohexylphosphino-2′,6′-diisopropoxybiphenyl, 2-di-tert-butylphosphino-3,4,5,6-tetramethyl-2′,4′,6′-triisopropyl-1,1′-biphenyl, Sodium 2′-dicyclohexylphosphino-2,6-dimethoxy-1,1′-biphenyl-3-sulfonate, 2-di-tert-butylphosphino-2′-methyl biphenyl, 2-dicyclohexylphosphino-2′-methylbiphenyl, 2′-(di-tert-butyl phosphino)-N,N-dimethyl biphenyl-2-amine, 2′-(diphenylphosphino)-N,N′-dimethyl-(1,1′-biphenyl)-2-amine, and the like, in the presence of a base such as sodium carbonate, lithium carbonate, potassium carbonate, cesium carbonate, sodium hydroxide, lithium hydroxide, potassium hydroxide, triethylamine, N,N-diisopropylethylamine, pyridine, 2,6-dimethylpyridine, and the like, in a solvent such as tetrahydrofuran, 1,4-dioxane, acetonitrile, methylene chloride, chloroform, 1,2-dichloroethane, 1,2-dimethoxyethane, and the like, optionally with heating, optionally with microwave irradiation to provide a compound of the formula (155). A compound of the formula (155) is reacted with an acid such as trifluoroacetic acid, hydrochloric acid, sulfuric acid, and the like, in a solvent such as methylene chloride, 1,2-dichloroethane, tetrahydrofuran, 1,4-dioxane, methanol, ethanol, N,N-dimethylformamide, dimethylsulfoxide, N,N-dimethylacetamide, and the like, optionally in the presence of methylphenyl ether, optionally with heating, optionally with microwave irradiation to provide a compound of the formula (156). A compound of the formula (156) is reacted with a compound of the formula (157) in the presence of a base such as triethylamine, N,N-diisopropylethylamine, pyridine, 2,6-dimethylpyridine, and the like, in the presence of a solvent such as N,N-dimethylformamide, dimethylsulfoxide, N,N-dimethylacetamide, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (158).

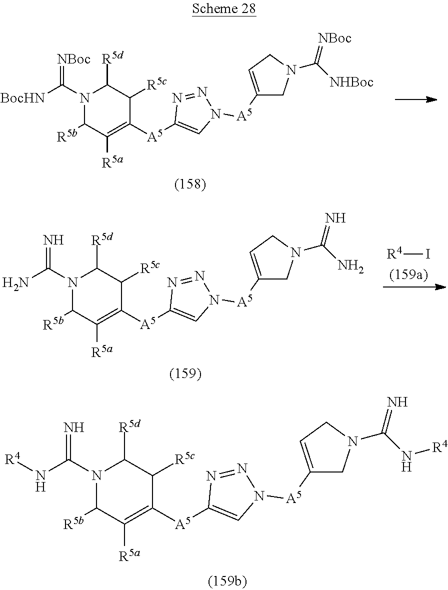

A compound of the formula (158) is reacted with an acid such as trifluoroacetic acid, hydrochloric acid, sulfuric acid, and the like, in a solvent such as methylene chloride, chloroform, 1,2-dichloroethane, tetrahydrofuran, 1,4-dioxane, methanol, ethanol, N,N-dimethylformamide, dimethylsulfoxide, N,N-dimethylacetamide, and the like, optionally in the presence of methylphenyl ether, optionally with heating, optionally with microwave irradiation to provide a compound of the formula (159). A compound of the formula (159) is reacted with a compound of the formula (159a), a known compound or a compound prepared by known methods, in the presence of a base such as sodium carbonate, potassium carbonate, lithium carbonate, triethylamine, pyridine, and the like, in a solvent such as N,N-dimethylformamide, dimethylsulfoxide, N,N-dimethylacetamide, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (159b).

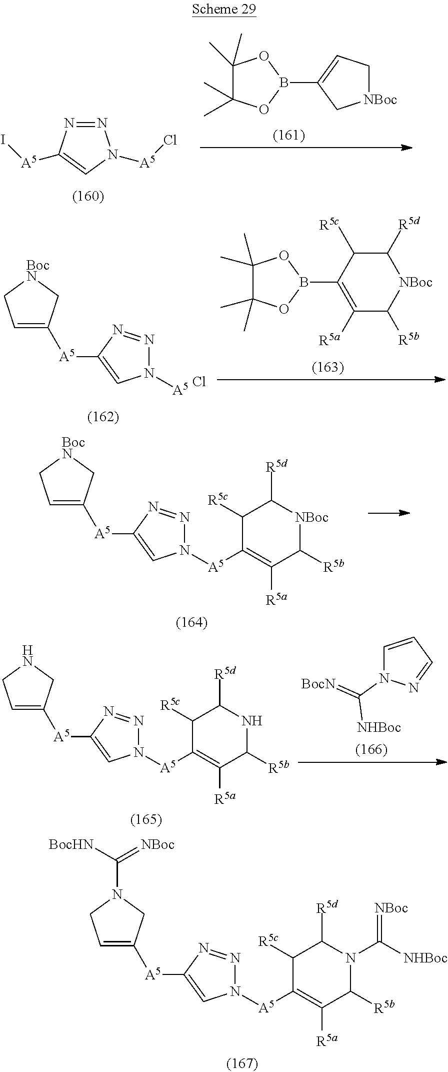

A compound of the formula (160) is reacted with a compound of the formula (161), a known compound or a compound prepared by known methods, in the presence of a palladium catalyst such as palladium (II) acetate, tetrakis(triphenylphosphine)palladium(0), dichlorobis (triphenylphosphine) palladium(II), palladium on carbon, bis(acetonitrile)dichloropalladium(II), and the like, in the presence of an organophosphine such as 2-dicyclohexylphosphino-2′,6′-dimethoxybiphenyl, 2-dicyclohexylphosphino-2′-(N,N-dimethylamino)biphenyl, 2-dicyclohexylphosphino-2′,4′,6′-triisopropylbiphenyl, 2-di-tert-butylphosphino-2′,4′,6′-triisopropylbiphenyl, (2-biphenyl)dicyclohexyl phosphine, (2-biphenyl)di-tert-butylphosphine, 2-dicyclohexylphosphino-2′,6′-diisopropoxybiphenyl, 2-di-tert-butylphosphino-3,4,5,6-tetramethyl-2′,4′,6′-tri isopropyl-1,1′-biphenyl, Sodium 2′-dicyclohexylphosphino-2,6-dimethoxy-1,1′-biphenyl-3-sulfonate, 2-di-tert-butylphosphino-2′-methyl biphenyl, 2-dicyclohexylphosphino-2″-methylbiphenyl, 2′-(di-tert-butylphosphino)-N,N-dimethyl biphenyl-2-amine, 1,2′-(diphenyphosphino)-N,N′-dimethyl-(1,1′-biphenyl)-2-amine, and the like, in the presence of a base such as sodium carbonate, lithium carbonate, potassium carbonate, cesium carbonate, sodium hydroxide, lithium hydroxide, potassium hydroxide, triethylamine, N,N-diisopropylethylamine, pyridine, 2,6-dimethylpyridine, and the like, in a solvent such as tetrahydrofuran, 1,4-dioxane, acetonitrile, methylene chloride, chloroform, 1,2-dichloroethane, 1,2-dimethoxyethane, and the like, optionally with heating, optionally with microwave irradiation to provide a compound of the formula (162). A compound of the formula (162) is reacted with a compound of the formula (163), a known compound or a compound prepared by known methods, in the presence of a palladium catalyst such as palladium (II) acetate, tetrakis(triphenylphosphine)palladium(0), dichlorobis (triphenylphosphine) palladium(II), palladium on carbon, bis(acetonitrile)dichloropalladium(II), and the like, in the presence of an organophosphine such as 2-dicyclohexylphosphino-2′,6′-dimethoxybiphenyl, 2-dicyclohexylphosphino-2′-(N,N-dimethylamino)biphenyl, 2-dicyclohexylphosphino-2′,4′,6′-triisopropylbiphenyl, 2-di-tert-butylphosphino-2′,4′,6′-triisopropylbiphenyl, (2-biphenyl)dicyclohexyl phosphine, (2-biphenyl)di-tert-butylphosphine, 2-dicyclohexylphosphino-2′,6′-diisopropoxybiphenyl, 2-di-tert-butylphosphino-3,4,5,6-tetramethyl-2′,4′,6′-triisopropyl-1,1′-biphenyl, Sodium 2′-dicyclohexylphosphino-2,6-dimethoxy-1,1′-biphenyl-3-sulfonate, 2-di-tert-butylphosphino-2′-methyl biphenyl, 2-dicyclohexylphosphino-2′-methylbiphenyl, 2′-(di-tert-butylphosphino)-N,N-dimethyl biphenyl-2-amine, 2′-(diphenylphosphino)-N,N′-dimethyl-(1,1′-biphenyl)-2-amine, and the like, in the presence of a base such as sodium carbonate, lithium carbonate, potassium carbonate, cesium carbonate, sodium hydroxide, lithium hydroxide, potassium hydroxide, triethylamine, N,N-diisopropylethylamine, pyridine, 2,6-dimethylpyridine, and the like, in a solvent such as tetrahydrofuran, 1,4-dioxane, acetonitrile, methylene chloride, chloroform, 1,2-dichloroethane, 1,2-dimethoxyethane, and the like, optionally with heating, optionally with microwave irradiation to provide a compound of the formula (164). A compound of the formula (164) is reacted with an acid such as trifluoroacetic acid, hydrochloric acid, sulfuric acid, and the like, in a solvent such as methylene chloride, 1,2-dichloroethane, tetrahydrofuran, 1,4-dioxane, methanol, ethanol, N,N-dimethylformamide, dimethylsulfoxide, N,N-dimethylacetamide, and the like, optionally in the presence of methylphenyl ether, optionally with heating, optionally with microwave irradiation to provide a compound of the formula (165). A compound of the formula (165) is reacted with a compound of the formula (166) in the presence of a base such as triethylamine, N,N-diisopropylethylamine, pyridine, 2,6-dimethylpyridine, and the like, in the presence of a solvent such as N,N-dimethylformamide, dimethylsulfoxide, N,N-dimethylacetamide, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (167).

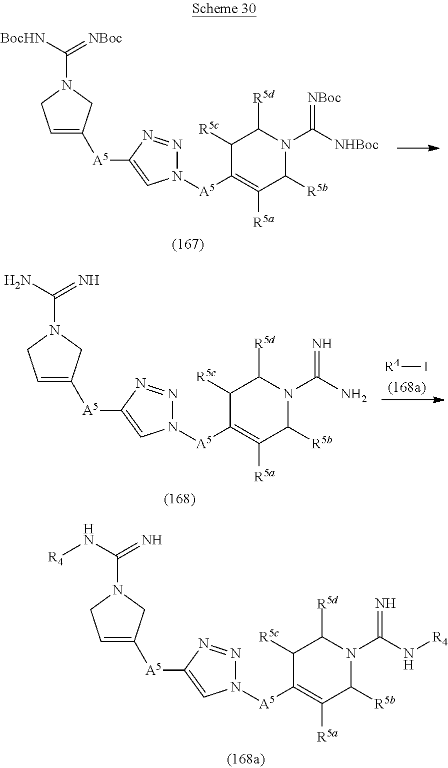

A compound of the formula (167) is reacted with an acid such as trifluoroacetic acid, hydrochloric acid, sulfuric acid, and the like, in a solvent such as methylene chloride, chloroform, 1,2-dichloroethane, tetrahydrofuran, 1,4-dioxane, methanol, ethanol, N,N-dimethylformamide, dimethylsulfoxide, N,N-dimethylacetamide, and the like, optionally in the presence of methylphenyl ether, optionally with heating, optionally with microwave irradiation to provide a compound of the formula (168). A compound of the formula (168) is reacted with a compound of the formula (168a), a known compound or a compound prepared by known methods, in the presence of a base such as sodium carbonate, potassium carbonate, lithium carbonate, triethylamine, pyridine, and the like, in a solvent such as N,N-dimethylformamide, dimethylsulfoxide, N,N-dimethylacetamide, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (168b).

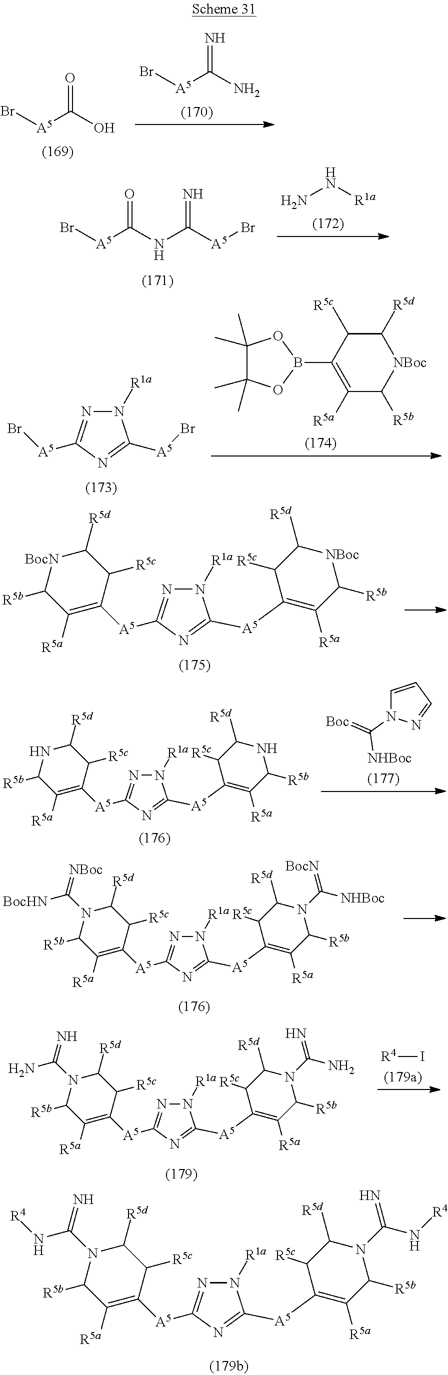

A compound of the formula (169), a known compound or a compound prepared by known methods, is reacted with a compound of the formula (170), a known compound or a compound prepared by known methods, in the presence of a coupling agent such as 1-[bis(dimethylamino)methylene]-1H-1,2,3-triazolo[4,5-b]pyridinium 3-oxid hexafluorophosphate, O-(benzotriazol-1-yl)-N,N,N′,N′-tetramethyluronium hexafluorophosphate, N,N′-dicyclohexylcarbodiimide, 1-ethyl-3-(3-dimethylaminopropyl) carbodiimide. and the like, optionally in the presence of hydroxybenzotriazole, optionally in the presence of 1-hydroxy-7-azabenzotriazole, and the like, in the presence of a base such as triethylamine. N,N-diisopropylethylamine, pyridine, 2,6-dimethylpyridine, and the like, in the presence of a solvent such as such as N-methyl-2-pyrrolidone, N,N-dimethylformamide, dimethylsulfoxide, N,N dimethylacetamide, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (171). A compound of the formula (171) is reacted with a compound of the formula (172), a known compound or a compound prepared by known methods, in the presence of an acid such as acetic acid, formic acid, trifluoroacetic acid, hydrochloric acid, sulfuric acid, and the like, optionally in a solvent such as methylene chloride, 1,2-dichloroethane, tetrahydrofuran, 1,4-dioxane, methanol, ethanol, N,N-dimethylformamide, dimethylsulfoxide, N,N-dimethylacetamide, and the like, optionally with heating, optionally with microwave irradiation to provide a compound of the formula (173). A compound of the formula (173) is reacted with a compound of the formula (174), a known compound or a compound prepared by known methods, in the presence of a palladium catalyst such as palladium (II) acetate, tetrakis(triphenylphosphine)palladium(0), dichlorobis (triphenylphosphine) palladium(II), palladium on carbon, bis(acetonitrile)dichloropalladium(II), and the like, in the presence of an organophosphine such as 2-dicyclohexylphosphino-2′,6′-dimethoxybiphenyl, 2-dicyclohexylphosphino-2′-(N,N-dimethyl amino)biphenyl, 2-dicyclohexylphosphino-2′,4′,6′-triisopropylbiphenyl, 2-di-tert-butylphosphino-2′,4′,6′-triisopropylbiphenyl, (2-biphenyl)dicyclohexyl phosphine, (2-biphenyl)di-tert-butylphosphine, 2-dicyclohexylphosphino-2′,6′-diisopropoxybiphenyl, 2-di-tert-butylphosphino-3,4,5,6-tetramethyl-2′,4′,6′-tri isopropyl-1,1′-biphenyl, Sodium 2′-dicyclohexylphosphino-2,6-dimethoxy-1,1′-biphenyl-3-sulfonate, 2-di-tert-butylphosphino-2′-methyl biphenyl, 2-dicyclohexylphosphino-2′-methylbiphenyl, 2′-(di-tert-butylphosphino)-N,N-dimethyl biphenyl-2-amine, 2′-(diphenylphosphino)-N,N′-dimethyl-(1,1′-biphenyl)-2-amine, and the like, in the presence of a base such as sodium carbonate, lithium carbonate, potassium carbonate, cesium carbonate, sodium hydroxide, lithium hydroxide, potassium hydroxide, triethylamine, N,N-diisopropylethylamine, pyridine, 2,6-dimethylpyridine, and the like, in a solvent such as tetrahydrofuran, 1,4-dioxane, acetonitrile, methylene chloride, chloroform, 1,2-dichloroethane, 1,2-dimethoxyethane, and the like, optionally with heating, optionally with microwave irradiation to provide a compound of the formula (175). A compound of the formula (175) is reacted with an acid such as trifluoroacetic acid, hydrochloric acid, sulfuric acid, and the like, in a solvent such as methylene chloride, 1,2-dichloroethane, tetrahydrofuran, 1,4-dioxane, methanol, ethanol, N,N-dimethylformamide, dimethylsulfoxide, N,N-dimethylacetamide, and the like, optionally in the presence of methylphenyl ether, optionally with heating, optionally with microwave irradiation to provide a compound of the formula (176). A compound of the formula (176) is reacted with a compound of the formula (177) in the presence of a base such as triethylamine, N,N-diisopropylethylamine, pyridine, 2,6-dimethylpyridine, and the like, in the presence of a solvent such as N,N-dimethylformamide, dimethylsulfoxide, N,N-dimethylacetamide, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (178). A compound of the formula (178) is reacted with an acid such as trifluoroacetic acid, hydrochloric acid, sulfuric acid, and the like, in a solvent such as methylene chloride, 1,2-dichloroethane, tetrahydrofuran, 1,4-dioxane, methanol, ethanol, N,N-dimethylformamide, dimethylsulfoxide, N,N-dimethylacetamide, and the like, optionally in the presence of methylphenyl ether, optionally with heating, optionally with microwave irradiation to provide a compound of the formula (179). A compound of the formula (179) is reacted with a compound of the formula (179a), a known compound or a compound prepared by known methods, in the presence of a base such as sodium carbonate, potassium carbonate, lithium carbonate, triethylamine, pyridine, and the like, in a solvent such as N,N-dimethylformamide, dimethylsulfoxide, N,N-dimethylacetamide, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (179b).

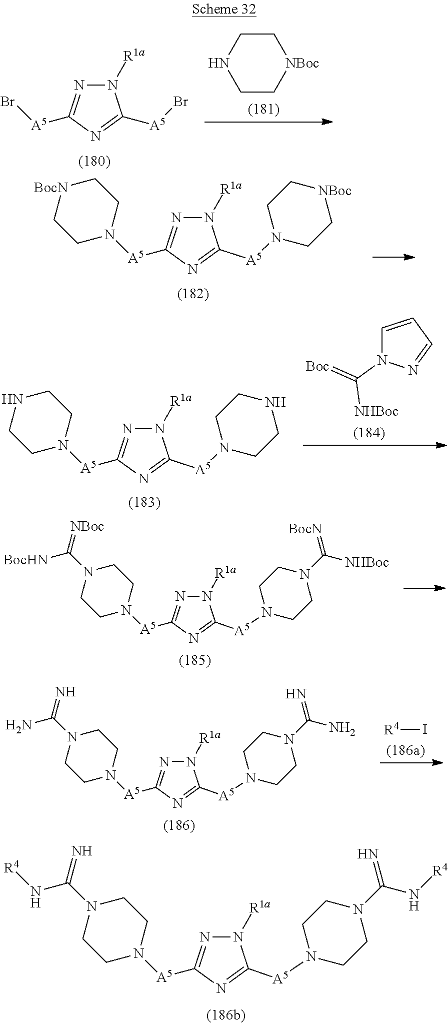

A compound of the formula (180) is reacted with a compound of the formula (181), a known compound or a compound prepared by known methods, in the presence of a palladium catalyst such as palladium (II) acetate, tetrakis(triphenylphosphine)palladium(0), dichlorobis (triphenylphosphine) palladium(II), palladium on carbon, bis(acetonitrile)dichloropalladium(II), and the like, in the presence of an organophosphine such as 2-dicyclohexylphosphino-2′,6′-dimethoxybiphenyl, 2-dicyclohexylphosphino-2′-(N,N-dimethylamino)biphenyl, 2-dicyclohexyl phosphino-2′,4′,6′-triisopropylbiphenyl, 2-di-tert-butylphosphino-2′,4′,6′-triisopropylbiphenyl, (2-biphenyl)dicyclohexyl phosphine, (2-biphenyl)di-tert-butylphosphine, 2-dicyclohexylphosphino-2′,6′-diisopropoxybiphenyl, 2-di-tert-butylphosphino-3,4,5,6-tetramethyl-2′,4′,6′-triisopropyl-1,1′-biphenyl, Sodium 2′-dicyclohexylphosphino-2,6-dimethoxy-1,1′-biphenyl-3-sulfonate, 2-di-tert-butylphosphino-2′-methyl biphenyl, 2-dicyclohexylphosphino-2′-methylbiphenyl, 2′-(di-tert-butylphosphino)-N,N-dimethyl biphenyl-2-amine, 2′-(diphenylphosphino)-N,N′-dimethyl-(1,1′-biphenyl)-2-amine, and the like, in the presence of a base such as sodium carbonate, lithium carbonate, potassium carbonate, cesium carbonate, sodium hydroxide, lithium hydroxide, potassium hydroxide, triethylamine, N,N-diisopropylethylamine, pyridine, 2,6-dimethylpyridine, and the like, in a solvent such as tetrahydrofuran, 1,4-dioxane, acetonitrile, methylene chloride, chloroform, 1,2-dichloroethane, 1,2-dimethoxyethane, and the like, optionally with heating, optionally with microwave irradiation to provide a compound of the formula (182). A compound of the formula (182) is reacted with an acid such as trifluoroacetic acid, hydrochloric acid, sulfuric acid, and the like, in a solvent such as methylene chloride, 1,2-dichloroethane, tetrahydrofuran, 1,4-dioxane, methanol, ethanol, N,N-dimethylformamide, dimethylsulfoxide, N,N-dimethylacetamide, and the like, optionally in the presence of methylphenyl ether, optionally with heating, optionally with microwave irradiation to provide a compound of the formula (183). A compound of the formula (183) is reacted with a compound of the formula (184) in the presence of a base such as triethylamine, N,N-diisopropylethylamine, pyridine, 2,6-dimethylpyridine, and the like, in the presence of a solvent such as N,N-dimethylformamide, dimethylsulfoxide, N,N-dimethylacetamide, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (185). A compound of the formula (185) is reacted with an acid such as trifluoroacetic acid, hydrochloric acid, sulfuric acid, and the like, in a solvent such as methylene chloride, 1,2-dichloroethane, tetrahydrofuran, 1,4-dioxane, methanol, ethanol, N,N-dimethylformamide, dimethylsulfoxide, N,N-dimethylacetamide, and the like, optionally in the presence of methylphenyl ether, optionally with heating, optionally with microwave irradiation to provide a compound of the formula (186). A compound of the formula (186) is reacted with a compound of the formula (186a), a known compound or a compound prepared by known methods, in the presence of a base such as sodium carbonate, potassium carbonate, lithium carbonate, triethylamine, pyridine, and the like, in a solvent such as N,N-dimethylformamide, dimethylsulfoxide, N,N-dimethylacetamide, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (186b).

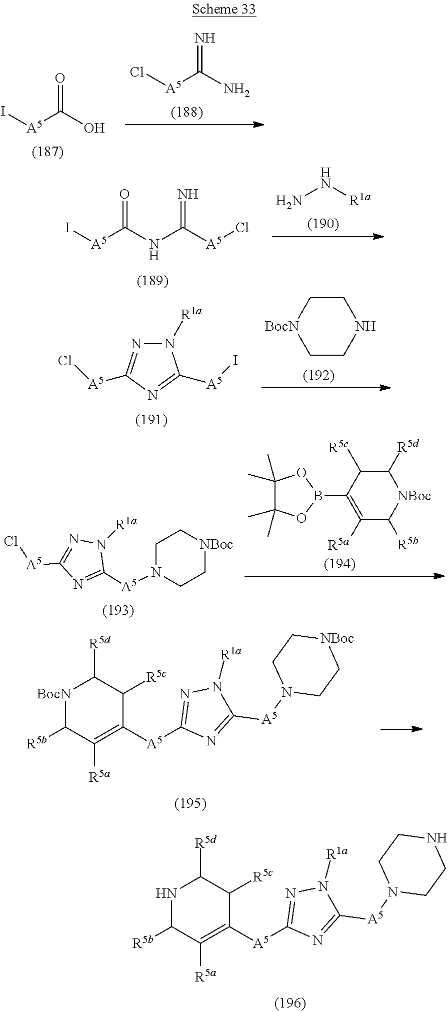

A compound of the formula (187), a known compound or a compound prepared by known methods, is reacted with a compound of the formula (188), a known compound or a compound prepared by known methods, in the presence of a coupling agent such as 1-[bis(dimethylamino)methylene]-1H-1,2,3-triazolo[4,5-b]pyridinium, 3-oxid hexafluorophosphate, O-(benzotriazol-1-yl)-N,N,N′,N′-tetramethyluronium hexafluorophosphate, N,N′-dicyclohexylcarbodiimide, 1-ethyl-3-(3-dimethylaminopropyl) carbodiimide. and the like, optionally in the presence of hydroxybenzotriazole, optionally in the presence of 1-hydroxy-7-azabenzotriazole, in the presence of a base such as triethylamine, N,N-diisopropylethylamine, pyridine, 2,6-dimethylpyridine, and the like, in the presence of a solvent such as such as N-methyl-2-pyrrolidone, N,N-dimethylformamide, dimethylsulfoxide, N,N-dimethylacetamide, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (189). A compound of the formula (189) is reacted with a compound of the formula (190), a known compound or a compound prepared by known methods, in the presence of an acid such as acetic acid, formic acid, trifluoroacetic acid, hydrochloric acid, sulfuric acid, and the like, optionally in a solvent such as methylene chloride, 1,2-dichloroethane, tetrahydrofuran, 1,4-dioxane, methanol, ethanol, N,N-dimethylformamide, dimethylsulfoxide, N,N-diacetylacetamide, and the like, optionally with heating, optionally with microwave irradiation to provide a compound of the formula (191). A compound of the formula (191) is reacted with a compound of the formula (192), a known compound or a compounds prepared by known methods, in the presence of a base such as sodium carbonate, lithium carbonate, potassium carbonate, cesium carbonate, sodium hydroxide, lithium hydroxide, potassium hydroxide, triethylamine, N,N-diisopropylethylamine, pyridine, 2,6-dimethylpyridine, and the like, in the presence of a solvent such as N-methyl-2-pyrrolidone, N,N-dimethylformamide, dimethylsulfoxide, N,N-dimethylacetamide, methylene chloride, chloroform, 1,2-dichloroethane, tetrahydrofuran, 1,4-dioxane, acetonitrile, 1,2-dimethoxyethane, and the like, optionally in the presence of methylphenyl ether, optionally with heating, optionally with microwave irradiation to provide a compound of the formula (193). A compound of the formula (193) is reacted with a compound of the formula (194), a known compound or a compound prepared by known methods, in the presence of a palladium catalyst such as palladium (II) acetate, tetrakis(triphenylphosphine)palladium(0), dichlorobis (triphenylphosphine) palladium(II), palladium on carbon, bis(acetonitrile)dichloropalladium(II), and the like, in the presence of an organophosphine such as 2-dicyclohexylphosphino-2′,6′-dimethoxybiphenyl, 2-dicyclohexylphosphino-2′-(N,N-dimethylamino)biphenyl, 2-dicyclohexylphosphino-2′,4′,6′-triisopropylbiphenyl, 2-di-tert-butylphosphino-2′,4′,6′-triisopropylbiphenyl, (2-biphenyl)dicyclohexyl phosphine, (2-biphenyl)di-tert-butylphosphine, 2-dicyclohexylphosphino-2′,6′-diisopropoxybiphenyl, 2-di-tert-butylphosphino-3,4,5,6-tetramethyl-2′,4′,6′-triisopropyl-1,1′-biphenyl, Sodium 2′-dicyclohexylphosphino-2,6-dimethoxy-1,1′-biphenyl-3-sulfonate, 2-di-tert-butylphosphino-2′-methyl biphenyl, 2-dicyclohexylphosphino-2′-methylbiphenyl, 2′-(di-tert-butylphosphino)-N,N-dimethyl biphenyl-2-amine, 2′-(diphenylphosphino)-N,N′-dimethyl-(1,1′-biphenyl)-2-amine, and the like, in the presence of a base such as sodium carbonate, lithium carbonate, potassium carbonate, cesium carbonate, sodium hydroxide, lithium hydroxide, potassium hydroxide, triethylamine, N,N-diisopropylethylamine, pyridine, 2,6-dimethylpyridine, and the like, in a solvent such as tetrahydrofuran, 1,4-dioxane, acetonitrile, methylene chloride, chloroform, 1,2-dichloroethane, 1,2-dimethoxyethane, and the like, optionally with heating, optionally with microwave irradiation to provide a compound of the formula (195). A compound of the formula (195) is reacted with an acid such as trifluoroacetic acid, hydrochloric acid, sulfuric acid, and the like, in a solvent such as methylene chloride, 1,2-dichloroethane, tetrahydrofuran, 1,4-dioxane, methanol, ethanol, N,N-dimethylformamide, dimethylsulfoxide, N,N-dimethylacetamide, and the like, optionally in the presence of methylphenyl ether, optionally with heating, optionally with microwave irradiation to provide a compound of the formula (196).

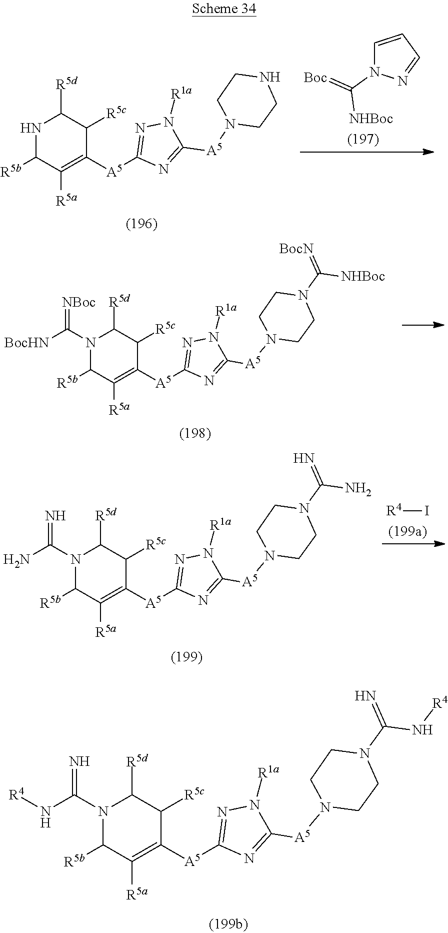

A compound of the formula (196) is reacted with a compound of the formula (197) in the presence of a base such as triethylamine, N,N-diisopropylethylamine, pyridine, 2,6-dimethylpyridine, and the like, in the presence of a solvent such as N,N-dimethylformamide, dimethylsulfoxide, N,N-dimethylacetamide, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (198). A compound of the formula (198) is reacted with an acid such as trifluoroacetic acid, hydrochloric acid, sulfuric acid, and the like, in a solvent such as methylene chloride, 1,2-dichloroethane, tetrahydrofuran, 1,4-dioxane, methanol, ethanol, N,N-dimethylformamide, dimethylsulfoxide, N,N-dimethylacetamide, and the like, optionally in the presence of methylphenyl ether, optionally with heating, optionally with microwave irradiation to provide a compound of the formula (199). A compound of the formula (199) is reacted with a compound of the formula (199a), a known compound or a compound prepared by known methods, in the presence of a base such as sodium carbonate, potassium carbonate, lithium carbonate, triethylamine, pyridine, and the like, in a solvent such as N,N-dimethylformamide, dimethylsulfoxide, N,N-dimethylacetamide, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (199b).

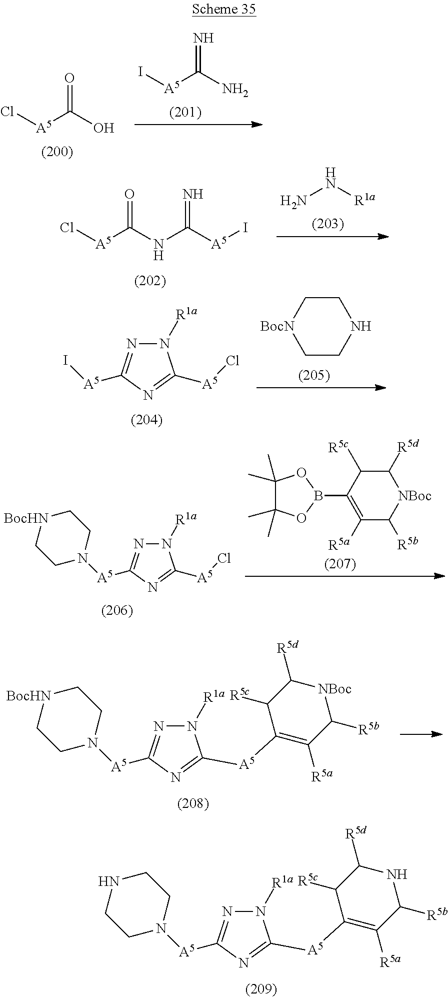

A compound of the formula (200), a known compound or a compound prepared by known methods, is reacted with a compound of the formula (201), a known compound or a compound prepared by known methods, in the presence of a coupling agent such as 1-[bis(dimethylamino)methylene]-1H-1,2,3-triazolo[4,5-b]pyridinium 3-oxid hexafluorophosphate, O-(benzotriazol-1-yl)-N,N,N′,N′-tetramethyluronium hexafluorophosphate, N,N′-dicyclohexylcarbodiimide, 1-ethyl-3-(3-dimethylaminopropyl) carbodiimide. and the like, optionally in the presence of hydroxybenzotriazole, optionally in the presence of 1-hydroxy-7-azabenzotriazole, and the like, in the presence of a base such as triethylamine, N,N-diisopropylethylamine, pyridine, 2,6-dimethylpyridine, and the like, in the presence of a solvent such as such as N-methyl-2-pyrolidone, N,N-dimethylformamide, dimethylsulfoxide, N,N-dimethylacetamide, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (202). A compound of the formula (202) is reacted with a compound of the formula (203), a known compound or a compound prepared by known methods, in the presence of an acid such as acetic acid, formic acid, trifluoroacetic acid, hydrochloric acid, sulfuric acid, and the like, optionally in a solvent such as methylene chloride, 1,2-dichloroethane, tetrahydrofuran, 1,4-dioxane, methanol, ethanol, N,N-dimethylformamide, dimethylsulfoxide, N,N-dimethylacetamide, and the like, optionally with heating, optionally with microwave irradiation to provide a compound of the formula (204). A compound of the formula (204) is reacted with a compound of the formula (205), a known compound or a compounds prepared by known methods, in the presence of a base such as sodium carbonate, lithium carbonate, potassium carbonate, cesium carbonate, sodium hydroxide, lithium hydroxide, potassium hydroxide, triethylamine, N,N-diisopropylethylamine, pyridine, 2,6-dimethylpyridine, and the like, in the presence of a solvent such as N-methyl-2-pyrrolidone, N,N-dimethylformamide, dimethylsulfoxide, N,N-dimethylacetamide, methylene chloride, chloroform, 1,2-dichloroethane, tetrahydrofuran, 1,4-dioxane, acetonitrile, 1,2-dimethoxyethane, and the like, optionally in the presence of methylphenyl ether, optionally with heating, optionally with microwave irradiation to provide a compound of the formula (206). A compound of the formula (206) is reacted with a compound of the formula (207), a known compound or a compound prepared by known methods, in the presence of a palladium catalyst such as palladium (II) acetate, tetrakis(triphenylphosphine)palladium(0), dichlorobis (triphenylphosphine) palladium(II), palladium on carbon, bis(acetonitrile)dichloropalladium(II), and the like, in the presence of an organophosphine such as 2-dicyclohexylphosphino-2′,6′-dimethoxybiphenyl, 2-dicyclohexylphosphino-2′-(N,N-dimethylamino)biphenyl, 2-di cyclohexyl phosphino-2′,4′,6′-triisopropylbiphenyl, 2-di-tert-butylphosphino-2′,4′,6′-triisopropylbiphenyl, (2-biphenyl)dicyclohexyl phosphine, (2-biphenyl)di-tert-butylphosphine, 2-dicyclohexylphosphino-2′,6′-diisopropoxybiphenyl, 2-di-tert-butylphosphino-3,4,5,6-tetramethyl-2′,4′,6′-triisopropyl-1,1′-biphenyl, Sodium 2′-dicyclohexylphosphino-2,6-dimethoxy-1,1′-biphenyl-3-sulfonate, 2-di-tert-butylphosphino-2′-methyl biphenyl, 2-dicyclohexylphosphino-2′-methylbiphenyl, 2′-(di-tert-butylphosphino)-N,N-dimethyl biphenyl-2-amine, 2′-(diphenylphosphino)-N,N′-dimethyl-(1,1′-biphenyl)-2-amine, and the like, in the presence of a base such as sodium carbonate, lithium carbonate, potassium carbonate, cesium carbonate, sodium hydroxide, lithium hydroxide, potassium hydroxide, triethylamine, N,N-diisopropylethylamine, pyridine, 2,6-dimethylpyridine, and the like, in a solvent such as tetrahydrofuran, 1,4-dioxane, acetonitrile, methylene chloride, chloroform, 1,2-dichloroethane, 1,2-dimethoxyethane, and the like, optionally with heating, optionally with microwave irradiation to provide a compound of the formula (208). A compound of the formula (208) is reacted with an acid such as trifluoroacetic acid, hydrochloric acid, sulfuric acid, and the like, in a solvent such as methylene chloride, 1,2-dichloroethane, tetrahydrofuran, 1,4-dioxane, methanol, ethanol, N,N-dimethylformamide, dimethylsulfoxide, N,N-dimethylacetamide, and the like, optionally in the presence of methylphenyl ether, optionally with heating, optionally with microwave irradiation to provide a compound of the formula (209).

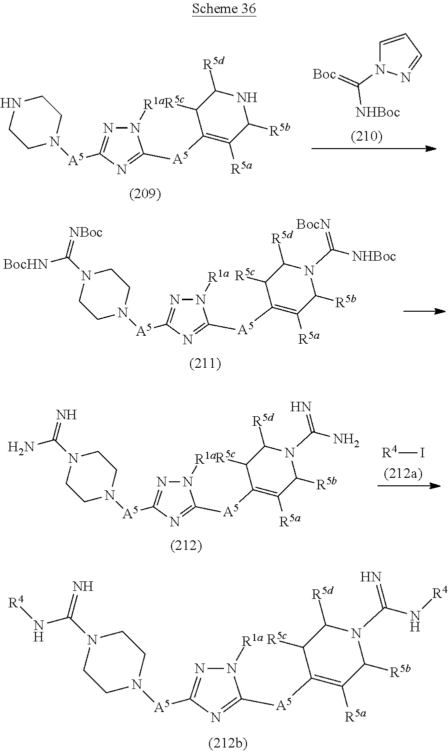

A compound of the formula (209) is reacted with a compound of the formula (210) in the presence of a base such as triethylamine, N,N-diisopropylethylamine, pyridine, 2,6-dimethylpyridine, and the like, in the presence of a solvent such as N,N-dimethylformamide, dimethylsulfoxide, N,N-dimethylacetamide, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (211). A compound of the formula (211) is reacted with an acid such as trifluoroacetic acid, hydrochloric acid, sulfuric acid, and the like, in a solvent such as methylene chloride, 1,2-dichloroethane, tetrahydrofuran, 1,4-dioxane, methanol, ethanol, N,N-dimethylformamide, dimethylsulfoxide, N,N-dimethylacetamide, and the like, optionally in the presence of methylphenyl ether, optionally with heating, optionally with microwave irradiation to provide a compound of the formula (212). A compound of the formula (212) is reacted with a compound of the formula (212a), a known compound or a compound prepared by known methods, in the presence of a base such as sodium carbonate, potassium carbonate, lithium carbonate, triethylamine, pyridine, and the like, in a solvent such as N,N-dimethylformamide, dimethylsulfoxide, N,N-dimethylacetamide, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (212b).

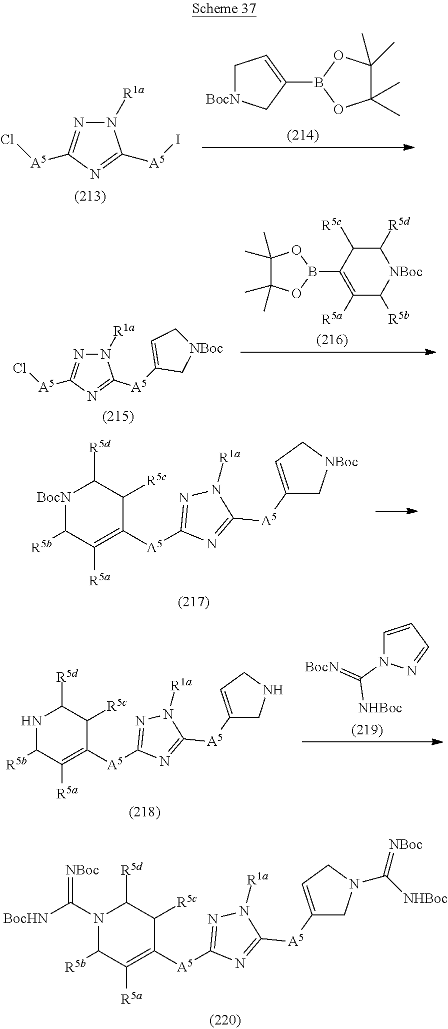

A compound of the formula (213) is reacted with a compound of the formula (214), a known compound or a compound prepared by known methods, in the presence of a palladium catalyst such as palladium (ii) acetate, tetrakis(triphenylphosphine)palladium(0), dichlorobis (triphenylphosphine) palladium(II), palladium on carbon, bis(acetonitrile)dichloropalladium(II), and the like, in the presence of an organophosphine such as 2-dicyclohexylphosphino-2′,6′-dimethoxybiphenyl, 2-dicyclohexylphosphino-2′-(N,N-dimethylamino)biphenyl, 2-dicyclohexylphosphino-2′,4′,6′-triisopropylbiphenyl, 2-di-tert-butylphosphino-2′,4′,6′-triisopropylbiphenyl, (2-biphenyl)dicyclohexyl phosphine, (2-biphenyl)di-tert-butylphosphine, 2-dicyclohexylphosphino-2′,6′-diisopropoxybiphenyl, 2-di-tert-butylphosphino-3,4,5,6-tetramethyl-2′,4′,6′-triisopropyl-1,1′-biphenyl, Sodium 2′-dicyclohexylphosphino-2,6-dimethoxy-1,1′-biphenyl-3-sulfonate, 2-di-tert-butylphosphino-T-methyl biphenyl, 2-dicyclohexylphosphino-2′-methylbiphenyl, 2′-(di-tert-butylphosphino)-N,N-dimethyl biphenyl-2-amine, 2′-(diphenylphosphino)-N,N′-dimethyl-(1,1′-biphenyl)-2-amine, and the like, in the presence of a base such as sodium carbonate, lithium carbonate, potassium carbonate, cesium carbonate, sodium hydroxide, lithium hydroxide, potassium hydroxide, triethylamine, N,N-diisopropylethylamine, pyridine, 2,6-dimethylpyridine, and the like, in a solvent such as tetrahydrofuran, 1,4-dioxane, acetonitrile, methylene chloride, chloroform, 1,2-dichloroethane, 1,2-dimethoxyethane, and the like, optionally with heating, optionally with microwave irradiation to provide a compound of the formula (215). A compound of the formula (215) is reacted with a compound of the formula (216), a known compound or a compound prepared by known methods, in the presence of a palladium catalyst such as palladium (II) acetate, tetrakis(triphenylphosphine)palladium(0), dichlorobis (triphenylphosphine) palladium(II), palladium on carbon, bis(acetonitrile)dichloropalladium(II), and the like, in the presence of an organophosphine such as 2-dicyclohexylphosphino-2′,6′-dimethoxybiphenyl, 2-dicyclohexylphosphino-2′-(N,N-dimethylamino)biphenyl, 2-dicyclohexylphosphino-2′,4′,6′-triisopropylbiphenyl, 2-di-tert-butylphosphino-2′,4′,6′-triisopropylbiphenyl, (2-biphenyl)dicyclohexyl phosphine, (2-biphenyl)di-tert-butylphosphine, 2-dicyclohexylphosphino-2′,6′-diisopropoxybiphenyl, 2-di-tert-butylphosphino-3,4,5,6-tetramethyl-2′,4′,6′-triisopropyl-1,1′-biphenyl, Sodium 2′-dicyclohexylphosphino-2,6-dimethoxy-1,1′-biphenyl-3-sulfonate, 2-di-tert-butylphosphino-2′-methyl biphenyl, 2-dicyclohexylphosphino-2′-methylbiphenyl, 2′-(di-tert-butylphosphino)-N,N-dimethyl biphenyl-2-amine, 2′-(diphenylphosphino)-N,N′-dimethyl-(1,1′-biphenyl)-2-amine, and the like, in the presence of a base such as sodium carbonate, lithium carbonate, potassium carbonate, cesium carbonate, sodium hydroxide, lithium hydroxide, potassium hydroxide, triethylamine, N,N-diisopropylethylamine, pyridine, 2,6-dimethylpyridine, and the like, in a solvent such as tetrahydrofuran, 1,4-dioxane, acetonitrile, methylene chloride, chloroform, 1,2-dichloroethane, 1,2-dimethoxyethane, and the like, optionally with heating, optionally with microwave irradiation to provide a compound of the formula (217). A compound of the formula (217) is reacted with an acid such as trifluoroacetic acid, hydrochloric acid, sulfuric acid, and the like, in a solvent such as methylene chloride, 1,2-dichloroethane, tetrahydrofuran, 1,4-dioxane, methanol, ethanol, N,N-dimethylformamide, dimethylsulfoxide, N,N-dimethylacetamide, and the like, optionally in the presence of methylphenyl ether, optionally with heating, optionally with microwave irradiation to provide a compound of the formula (218). A compound of the formula (218) is reacted with a compound of the formula (219) in the presence of a base such as triethylamine, N,N-diisopropylethylamine, pyridine, 2,6-dimethylpyridine, and the like, in the presence of a solvent such as N,N-dimethylformamide, dimethylsulfoxide, N,N-dimethylacetamide, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (220).

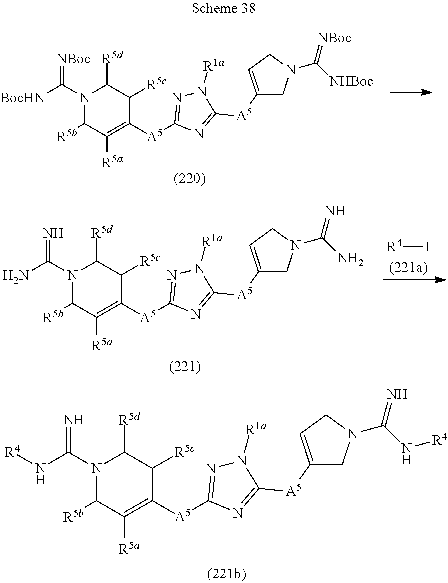

A compound of the formula (220) is reacted with an acid such as trifluoroacetic acid, hydrochloric acid, sulfuric acid, and the like, in a solvent such as methylene chloride, 1,2-dichloroethane, tetrahydrofuran, 1,4-dioxane, methanol, ethanol, INN-dimethylformamide, dimethylsulfoxide, N,N-dimethylacetamide, and the like, optionally in the presence of methylphenyl ether, optionally with heating, optionally with microwave irradiation to provide a compound of the formula (221). A compound of the formula (221) is reacted with a compound of the formula (221a), a known compound or a compound prepared by known methods, in the presence of a base such as sodium carbonate, potassium carbonate, lithium carbonate, triethylamine, pyridine, and the like, in a solvent such as N,N-dimethylformamide, dimethylsulfoxide, N,N-dimethylacetamide, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (221b).

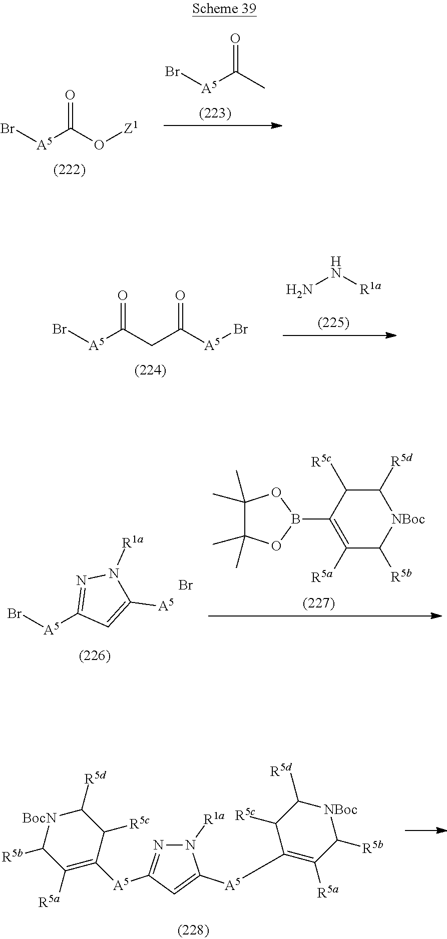

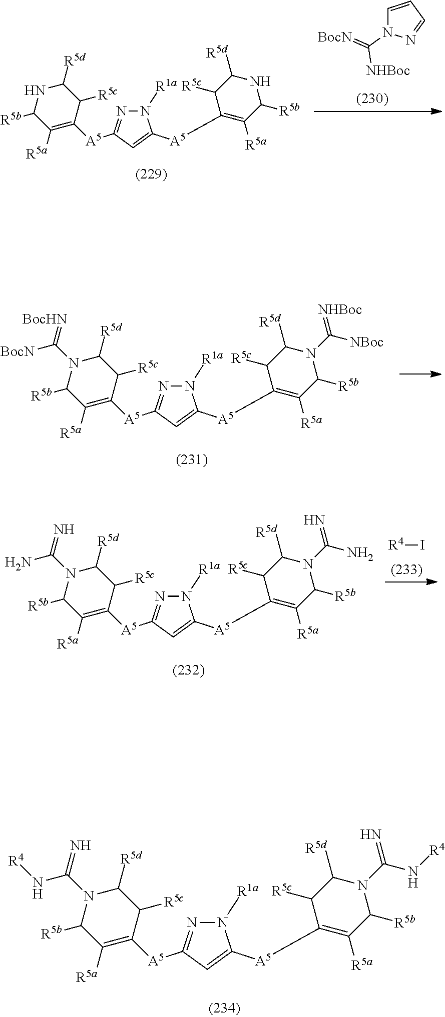

A compound of the formula (222), a known compound or a compound prepared by known methods wherein Z¹ is C₁-C₄ alkyl, is reacted with a compound of the formula (223), a known compound or a compound prepared by known methods, in the presence of a base such as sodium hydride, potassium hydride, lithium diisopropylamide, sodium di isopropylamide, potassium diisopropylamide, lithium bis(trimethylsilylamide, sodium bis(trimethylsilyl)amide, potassium bis(trimethylsilyl)amide, and the like in an organic solvent such as tetrahydrofuran, 1,4-dioxane, 1,2-dimethoxyethane, dimethylformamide, dimethylacetamide, methylene chloride, 1,2-dichloroethan, and the like, optionally with heating, optionally with microwave irradiation, to provide a compound of the formula (224). A compound of the formula (224) is reacted with a compound of the formula (225), a known compound or a compound prepared by known methods, in the presence of an acid such as acetic acid, formic acid, trifluoroacetic acid, hydrochloric acid, sulfinic acid, and the like, solvent such as methanol, ethanol, isopropanol, methylene chloride, chloroform, 1,2-dichloroethane, tetrahydrofuran, 1,4-dioxane, acetonitrile, 1,2-dimethoxyethane, and the like, optionally with heating, optionally with microwave irradiation, to provide a compound of the formula (226). A compound of the formula (226) is reacted with a compound of the formula (227), a known compound or a compound prepared by known methods, in the presence of a palladium catalyst such as palladium (II) acetate, tetrakis(triphenylphosphine)palladium(0), dichlorobis (triphenylphosphine) palladium(II), palladium on carbon, bis(acetonitrile)dichloropalladium(II), and the like, in the presence of an organophosphine such as 2-dicyclohexylphosphino-2′,6′-dimethoxybiphenyl, 2-dicyclohexylphosphino-2′-(N,N-dimethylamino)biphenyl, 2-dicyclohexylphosphino-2′,4′,6′-triisopropylbiphenyl, 2-di-tert-butylphosphino-2′,4′,6′-triisopropylbiphenyl, (2-biphenyl)dicyclohexyl phosphine, (2-biphenyl)di-tert-butylphosphine, 2-dicyclohexylphosphino-2′,6′-diisopropoxybiphenyl, 2-di-tert-butylphosphino-3,4,5,6-tetramethyl-2′,4′,6′-triisopropyl-1,1′-biphenyl. Sodium 2′-dicyclohexylphosphino-2,6-dimethoxy-1,1′-biphenyl-3-sulfonate, 2-di-tert-butylphosphino-2′-methyl biphenyl, 2-dicyclohexylphosphino-2′-methylbiphenyl, 2′-(di-tert-butylphosphino)-N,N-dimethyl biphenyl-2-amine, 2′-(diphenylphosphino)-N,N′-dimethyl-(1,1′-biphenyl)-2-amine, and the like, in the presence of a base such as sodium carbonate, lithium carbonate, potassium carbonate, cesium carbonate, sodium hydroxide, lithium hydroxide, potassium hydroxide, triethylamine, N,N-diisopropylethylamine, pyridine, 2,6-dimethylpyridine, and the like, in a solvent such as tetrahydrofuran, 1,4-dioxane, acetonitrile, methylene chloride, chloroform, 1,2-dichloroethane, 1,2-dimethoxyethane, and the like, optionally with heating, optionally with microwave irradiation to provide a compound of the formula (228). A compound of the formula (228) is reacted with an acid such as trifluoroacetic acid, hydrochloric acid, sulfuric acid, and the like, in a solvent such as methylene chloride, 1,2-dichloroethane, tetrahydrofuran, 1,4-dioxane, methanol, ethanol, N,N-dimethylfomamide, dimethylsulfoxide, N,N-dimethylacetamide, and the like, optionally in the presence of methylphenyl ether, optionally with heating, optionally with microwave irradiation to provide a compound of the formula (229). A compound of the formula (229) is reacted with a compound of the formula (230) in the presence of a base such as triethylamine, N,N-diisopropylethylamine, pyridine, 2,6-dimethylpyridine, and the like, in the presence of a solvent such as N,N-dimethylformamide, dimethylsulfoxide, N,N-dimethylacetamide, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (231). A compound of the formula (231) is reacted with an acid such as trifluoroacetic acid, hydrochloric acid, sulfuric acid, and the like, in a solvent such as methylene chloride, 1,2-dichloroethane, tetrahydrofuran, 1,4-dioxane, methanol, ethanol, N,N-dimethylformamide, dimethylsulfoxide, N,N-dimethylacetamide, and the like, optionally in the presence of methylphenyl ether, optionally with heating, optionally with microwave irradiation to provide a compound of the formula (232). A compound of the formula (232) is reacted with a compound of the formula (233), a known compound or a compound prepared by known methods, in the presence of a base such as sodium carbonate, potassium carbonate, lithium carbonate, triethylamine, pyridine, and the like, in a solvent such as N,N-dimethylformamide, dimethylsulfoxide, N,N-dimethylacetamide, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (234).

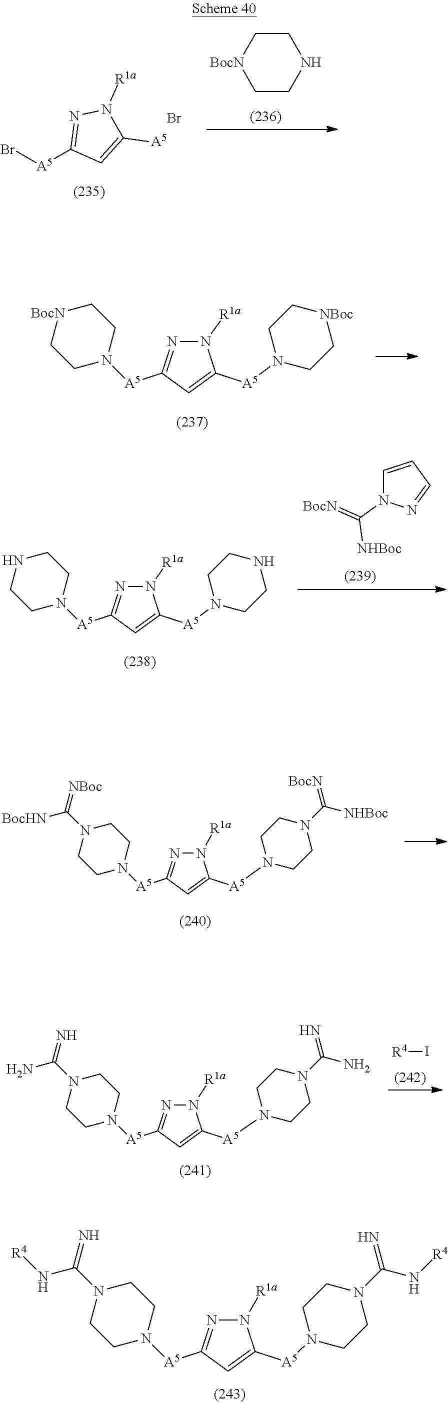

A compound of the formula (235) is reacted with a compound of the formula (236), a known compound or a compound prepared by known methods, in the presence of a palladium catalyst such as palladium (II) acetate-tetrakis(triphenylphosphine)palladium(0), dichlorobis (triphenylphosphine) palladium(II), palladium on carbon, his(acetonitrile)dichloropalladium (ii), and the like, in the presence of an organophosphine such as 2-dicyclohexylphosphino-2′,6′-dimethoxybiphenyl, 2-dicyclohexylphosphino-2′-(N,N-dimethylamino)biphenyl, 2-dicyclohexylphosphino-2′,4′,6′-triisopropylbiphenyl, 2-di-tert-butylphosphino-2′,4′,6′-triisopropylbiphenyl, (2-biphenyl)dicyclohexyl phosphine, (2-biphenyl)di-tert-butylphosphine, 2-dicyclohexylphosphino-2′,6′-diisopropoxybiphenyl, 2-di-tert-butylphosphino-3,4,5,6-tetramethyl-2′,4′,6′-triisopropyl-1,1′-biphenyl, Sodium 2′-dicyclo hexylphosphino-2,6-dimethoxy-1,1′-biphenyl-3-sulfonate, 2-di-tert-butylphosphino-2′-methyl biphenyl, 2-dicyclohexylphosphino-2′-methylbiphenyl, 2′-(di-tert-butylphosphino)-N,N-dimethyl biphenyl-2-amine, 2′-(diphenylphosphino)-N,N′-dimethyl-(1,1′-biphenyl)-2-amine, and the like, in the presence of a base such as sodium carbonate, lithium carbonate, potassium carbonate, cesium carbonate, sodium hydroxide, lithium hydroxide, potassium hydroxide, triethylamine, N,N-diisopropylethylamine, pyridine, 2,6-dimethylpyridine, and the like, in a solvent such as tetrahydrofuran, 1,4-dioxane, acetonitrile, methylene chloride, chloroform, 1,2-dichloroethane, 1,2-dimethoxyethane, and the like, optionally with heating, optionally with microwave irradiation to provide a compound of the formula (237). A compound of the formula (237) is reacted with an acid such as trifluoroacetic acid, hydrochloric acid, sulfuric acid, and the like, in a solvent such as methylene chloride, 1,2-dichloroethane, tetrahydrofuran, 1,4-dioxane, methanol, ethanol, N,N-dimethylformamide, dimethylsulfoxide, N,N-dimethylacetamide, and the like, optionally in the presence of methylphenyl ether, optionally with heating, optionally with microwave irradiation to provide a compound of the formula (238). A compound of the formula (238) is reacted with a compound of the formula (239) in the presence of a base such as triethylamine, N,N-diisopropylethylamine, pyridine, 2,6-dimethylpyridine, and the like, in the presence of a solvent such as N,N-dimethylformamide, dimethylsulfoxide, N,N-dimethylacetamide, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (240). A compound of the formula (240) is reacted with an acid such as trifluoroacetic acid, hydrochloric acid, sulfuric acid, and the like, in a solvent such as methylene chloride, chloroform, 1,2-dichloroethane, tetrahydrofuran, 1,4-dioxane, methanol, ethanol. N,N-dimethylformamide, dimethylsulfoxide, N,N-dimethylacetamide, and the like, optionally in the presence of methylphenyl ether, optionally with heating, optionally with microwave irradiation to provide a compound of the formula (241). A compound of the formula (241) is reacted with a compound of the formula (242), a known compound or a compound prepared by known methods, in the presence of a base such as sodium carbonate, potassium carbonate, lithium carbonate, triethylamine, pyridine, and the like, in a solvent such as N,N-dimethylformamide, dimethylsulfoxide, N,N-dimethylacetamide, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (243).

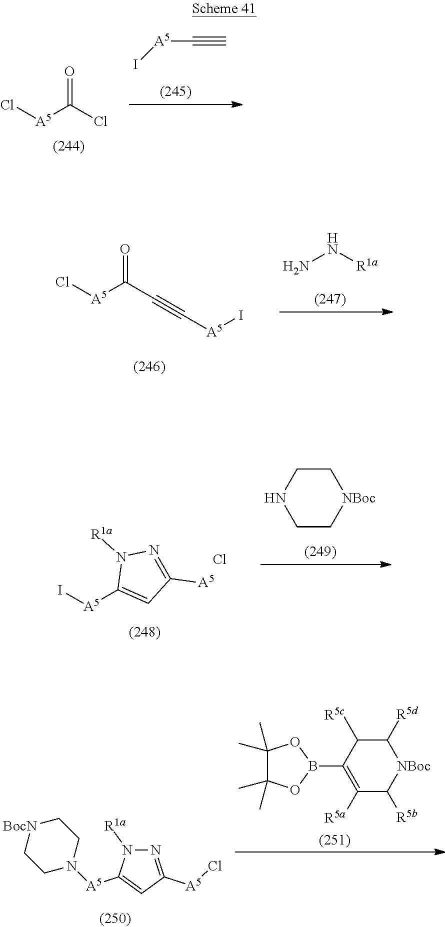

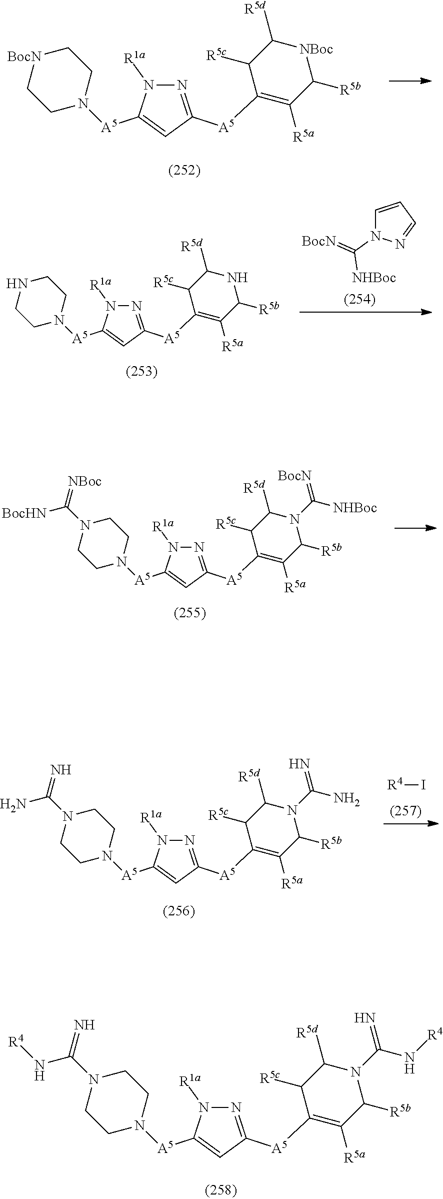

A compound of the formula (244), a known compound or a compound prepared by known methods, is reacted with a compound of the formula (245) a known compound or a compound prepared by known methods, in the presence of copper iodide, in the presence of a base such as triethylamine, N,N-diisopropylethylamine, pyridine, 2,6-dimethylpyridine, and the like, in the presence of a palladium catalyst such as palladium (II) acetate, tetrakis(triphenylphosphine)palladium(0), dichlorobis (triphenylphosphine) palladium(II), palladium on carbon, bis(acetonitrile)dichloropalladium(II), and the like, in the presence of a solvent such as methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (246). A compound of the formula (246) is reacted with a compound of the formula (247), a known compound or a compound prepared by known methods, in the presence of a solvent such as methanol, ethanol, isopropanol, methylene chloride, chloroform, 1,2-dichloroethane, tetrahydrofuran, 1,4-diaxane, acetonitrile, 1,2′-dimethoxyethane, and the like, optionally with heating, optionally with microwave eradiation to provide a compound of the formula (248). A compound of the formula (248) is reacted with of the formula (249), a known compound or a compound prepared by known methods, in the presence of a base such as sodium carbonate, lithium carbonate, potassium carbonate, cesium carbonate, sodium hydroxide, lithium hydroxide, potassium hydroxide, triethylamine, N,N-diisopropylethylamine, pyridine, 2,6-dimethylpyridine, and the like, in the presence of a solvent such as N-methyl-2-pyrrolidone, N,N-dimethylformamide, dimethylsulfoxide, N,N-dimethylacetamide, methylene chloride, chloroform, 1,2-dichloroethane, tetrahydrofuran, 1,4-dioxane, acetonitrile, 1,2-dimethoxyethane, and the like, optionally in the presence of methylphenyl ether, optionally with heating, optionally with microwave irradiation to provide a compound of the formula (250). A compound of the formula (250) is reacted with a compound of the formula (251), a known compound or a compound prepared by known methods, in the presence of a palladium catalyst such as palladium (II) acetate, tetrakis(triphenylphosphine)palladium(0), dichlorobis (triphenylphosphine) palladium(II), palladium on carbon, bis(acetonitrile)dichloropalladium(II), and the like, in the presence of an organophosphine such as 2-dicyclohexylphosphino-2′,6′-dimethoxybiphenyl, 2-dicyclohexylphosphino-2′-(N,N-dimethylamino)biphenyl, 2-dicyclohexylphosphino-2′,4′,6′-triisopropylbiphenyl, 2-di-tert-butylphosphino-2′,4′,6′-triisopropylbiphenyl, (2-biphenyl)dicyclohexyl phosphine, (2-biphenyl)di-tert-butylphosphine, 2-dicyclohexylphosphino-2′,6′-diisopropoxybiphenyl, 2-di-tert-butylphosphino-3,4,5,6-tetramethyl-2′,4′,6′-triisopropyl-1,1′-biphenyl. Sodium 2′-dicyclohexylphosphino-2,6-dimethoxy-1,1′-biphenyl-3-sulfonate, 2-di-tert-butylphosphino-2′-methyl biphenyl, 2-dicyclohexylphosphino-2′-methylbiphenyl, 2′-(di-tert-butylphosphino)-N,N-dimethyl biphenyl-2-amine, 2′-(diphenylphosphino)-N,N′-dimethyl-(1,1′-biphenyl)-2-amine, and the like, in the presence of a base such as sodium carbonate, lithium carbonate, potassium carbonate, cesium carbonate, sodium hydroxide, lithium hydroxide, potassium hydroxide, triethylamine, N,N-diisopropylethylamine, pyridine, 2,6-dimethylpyridine, and the like, in a solvent such as tetrahydrofuran, 1,4-dioxane, acetonitrile, methylene chloride, chloroform, 1,2-dichloroethane, 1,2-dimethoxyethane, and the like, optionally with heating, optionally with microwave irradiation to provide a compound of the formula (252). A compound of the formula (252) is reacted with an acid such as trifluoroacetic acid, hydrochloric acid, sulfuric acid, and the like, in a solvent such as methylene chloride, 1,2-dichloroethane, tetrahydrofuran, 1,4-dioxane, methanol, ethanol, N,N-dimethylformamide, dimethylsulfoxide, N,N-dimethylacetamide, and the like, optionally in the presence of methylphenyl ether, optionally with heating, optionally with microwave irradiation to provide a compound of the formula (253). A compound of the formula (253) is reacted with a compound of the formula (254) in the presence of a base such as triethylamine, N,N-diisopropylethylamine, pyridine, 2,6-dimethylpyridine, and the like, in the presence of a solvent such as N,N-dimethylformamide, dimethylsulfoxide, N,N-dimethylacetamide, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (255). A compound of the formula (255) is reacted with an acid such as trifluoroacetic acid, hydrochloric acid, sulfuric acid, and the like, in a solvent such as methylene chloride, chloroform, 1,2-dichloroethane, tetrahydrofuran, 1,4-dioxane, methanol, ethanol, N,N-dimethylformamide, dimethylsulfoxide, N,N-dimethylacetamide, and the like, optionally in the presence of methylphenyl ether, optionally with heating, optionally with microwave irradiation to provide a compound of the formula (256). A compound of the formula (256) is reacted with a compound of the formula (257), a known compound or a compound prepared by known methods, in the presence of a base such as sodium carbonate, potassium carbonate, lithium carbonate, triethylamine, pyridine, and the like, in a solvent such as N,N-dimethylformamide, dimethylsulfoxide, N,N-dimethylacetamide, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (258).

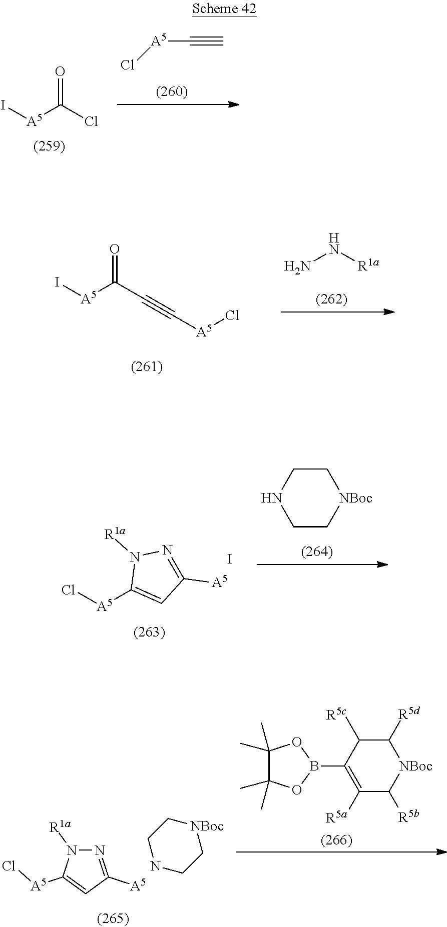

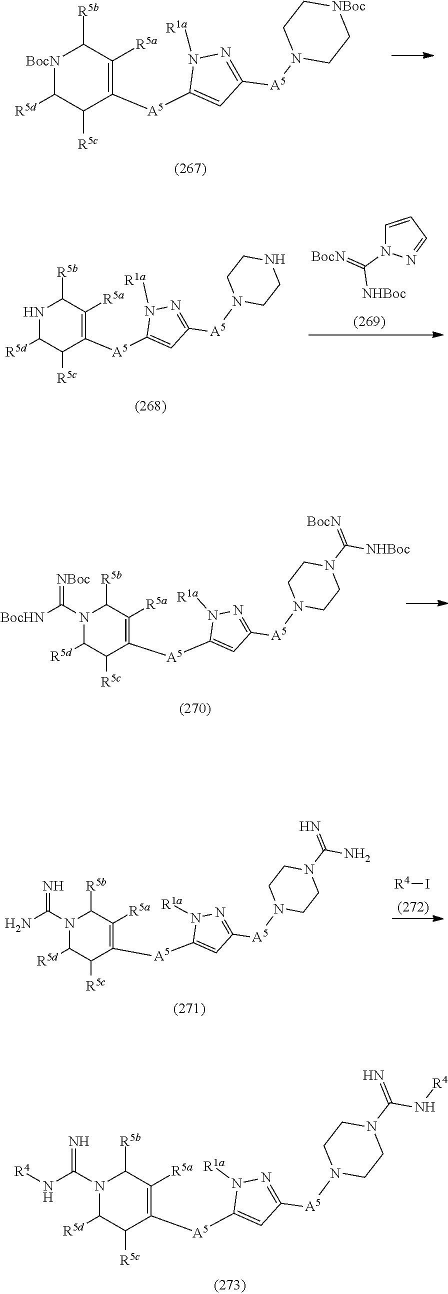

A compound of the formula (259), a known compound or a compound prepared by known methods, is reacted with a compound of the formula (260) a known compound or a compound prepared by known methods, in the presence of copper iodide, in the presence of a base such as triethylamine, N,N-diisopropylethylamine, pyridine, 2,6-dimethylpyridine, and the like, in the presence of a palladium catalyst such as palladium (II) acetate, tetrakis(triphenylphosphine)palladium(0), dichlorobis (triphenylphosphine) palladium(II), palladium on carbon, bis(acetonitrile)dichloropalladium(II) and the like, in the presence of a solvent such as methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (261). A compound of the formula (261) is reacted with a compound of the formula (262), a known compound or a compound prepared by known methods, in the presence of a solvent such as methanol, ethanol, isopropanol, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (263). A compound of the formula (263) is reacted with of the formula (264), a known compound or a compound prepared by known methods, in the presence of a base such as sodium carbonate, lithium carbonate, potassium carbonate, cesium carbonate, sodium hydroxide, lithium hydroxide, potassium hydroxide, triethylamine, N,N-diisopropylethylamine, pyridine, 2,6-dimethylpyridine, and the like, in the presence of a solvent such as N-methyl-2-pyrrolidone, N,N-dimethylformamide, dimethylsulfoxide, N,N-dimethylacetamide, methylene chloride, chloroform, 1,2-dichloroethane, tetrahydrofuran, 1,4-dioxane, acetonitrile, 1,2-dimethoxyethane, and the like, optionally in the presence of methylphenyl ether, optionally with heating, optionally with microwave irradiation to provide a compound of the formula (265). A compound of the formula (265) is reacted with a compound of the formula (266), a known compound or a compound prepared by known methods, in the presence of a palladium catalyst such as palladium (II) acetate, tetrakis(triphenylphosphine)palladium(0), dichlorobis (triphenylphosphine) palladium(II), palladium on carbon, bis(acetonitrile)dichloropalladium(II), and the like, in the presence of an organophosphine such as 2-dicyclohexylphosphino-2′,6′-dimethoxybiphenyl, 2-dicyclohexylphosphino-2′-(N,N-dimethylamino)biphenyl, 2-dicyclohexylphosphino-2′,4′,6′-triisopropylbiphenyl, 2-di-tert-butylphosphino-2′,4′,6′-triisopropylbiphenyl, (2-biphenyl)dicyclohexyl phosphine, (2-biphenyl)di-tert-butylphosphine, 2-dicyclohexylphosphino-2′,6′-diisopropoxybiphenyl, 2-di-tert-butylphosphino-3,4,5,6-tetramethyl-2′,4′,6′-triisopropyl-1,1′-biphenyl, Sodium 2′-dicyclohexylphosphino-2,6-dimethoxy-1,1′-biphenyl-3-sulfonate, 2-di-tert-butylphosphino-2′-methyl biphenyl, 2-dicyclohexylphosphino-2′-methylbiphenyl, 2′-(di-tert-butylphosphino)-N,N-dimethyl biphenyl-2-amine, 2′-(diphenylphosphino)-N,N′-dimethyl-(1,1′-biphenyl)-2-amine, and the like, in the presence of a base such as sodium carbonate, lithium carbonate, potassium carbonate, cesium carbonate, sodium hydroxide, lithium hydroxide, potassium hydroxide, triethylamine, N,N-diisopropylethylamine, pyridine, 2,6-dimethylpyridine, and the like, in a solvent such as tetrahydrofuran, 1,4-dioxane, acetonitrile, methylene chloride, chloroform, 1,2-dichloroethane, 1,2-dimethoxyethane, and the like, optionally with heating, optionally with microwave irradiation to provide a compound of the formula (267). A compound of the formula (267) is reacted with an acid such as trifluoroacetic acid, hydrochloric acid, sulfuric acid, and the like, in a solvent such as methylene chloride, 1,2-dichloroethane, tetrahydrofuran, 1,4-dioxane, methanol, ethanol, N,N-dimethylformamide, dimethylsulfoxide, N,N-dimethylacetamide, and the like, optionally in the presence of methylphenyl ether, optionally with heating, optionally with microwave irradiation to provide a compound of the formula (268). A compound of the formula (268) is reacted with a compound of the formula (269) in the presence of a base such as triethylamine, N,N-diisopropylethylamine, pyridine, 2,6-dimethylpyridine, and the like, in the presence of a solvent such as N,N-dimethylformamide, dimethylsulfoxide, N,N-dimethylacetamide, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (270). A compound of the formula (270) is reacted with an acid such as trifluoroacetic acid, hydrochloric acid, sulfuric acid, and the like, in a solvent such as methylene chloride, chloroform, 1,2-dichloroethane, tetrahydrofuran, 1,4-dioxane, methanol, ethanol, N,N-dimethylformamide, dimethylsulfoxide, N,N-dimethylacetamide, and the like, optionally in the presence of methylphenyl ether, optionally with heating, optionally with microwave irradiation to provide a compound of the formula (271). A compound of the formula (271) is reacted with a compound of the formula (272), a known compound or a compound prepared by known methods, in the presence of a base such as sodium carbonate, potassium carbonate, lithium carbonate, triethylamine, pyridine, and the like, in a solvent such as N,N-dimethylformamide, dimethylsulfoxide, N,N-dimethylacetamide, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (273).

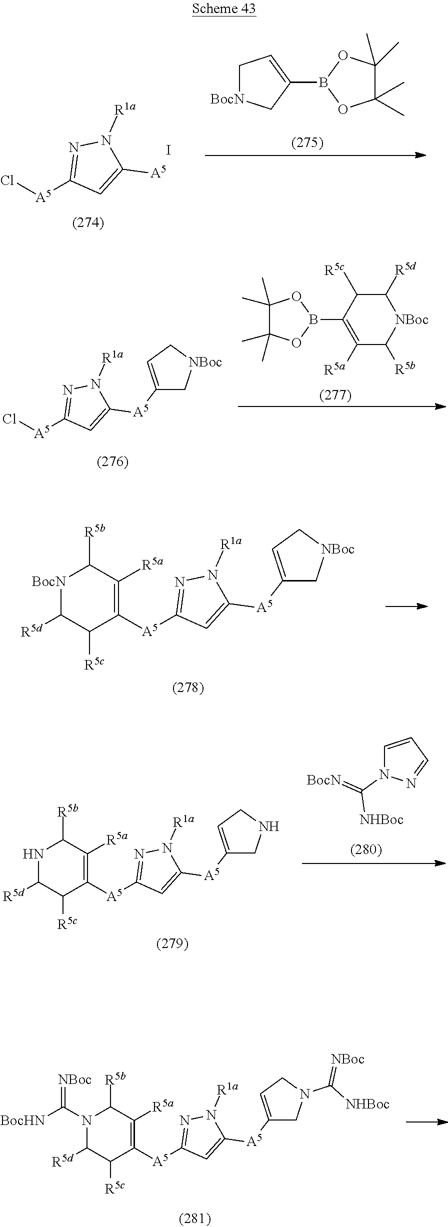

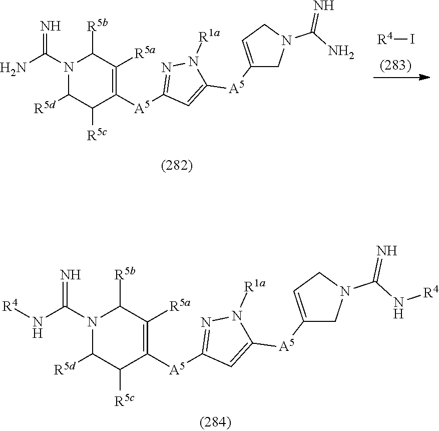

A compound of the formula (274) is reacted with a compound of the formula (275), a known compound or a compound prepared by known methods, in the presence of a palladium catalyst such as palladium (II) acetate, tetrakis(triphenylphosphine)palladium(0), dichlorobis (triphenylphosphine) palladium(II) palladium on carbon, bis(acetonitrile)dichloropalladium(II), and the like, in the presence of an organophosphine such as 2-dicyclohexylphosphino-2′,6′-dimethoxybiphenyl, 2-dicyclohexylphosphino-2′-(N,N-dimethylamino)biphenyl, 2-dicyclohexylphosphino-2,4′,6′-triisopropylbiphenyl, 2-di-tert-butylphosphino-2′,4′,6′-triisopropylbiphenyl, (2-biphenyl)dicyclohexyl phosphine, (2-biphenyl)di-tert-butylphosphine, 2-dicyclohexylphosphino-2′,6′-diisopropoxybiphenyl, 2-di-tert-butylphosphino-3,4,5,6-tetramethyl-2′,4′,6′-triisopropyl-1,1′-biphenyl, Sodium 2′-dicyclohexylphosphino-2,6-dimethoxy-1,1′-biphenyl-3-sulfonate, 2-di-test-butylphosphino-2′-methyl biphenyl, 2-dicyclohexylphosphino-2′-methylbiphenyl, 2′-(ditert-butylphosphino)-N,N-dimethyl biphenyl-2-amine, 2′-(diphenylphosphino)-N,N′-dimethyl-(1,1′-biphenyl)-2-amine, and the like, in the presence of a base such as sodium carbonate, lithium carbonate, potassium carbonate, cesium carbonate, sodium hydroxide, lithium hydroxide, potassium hydroxide, triethylamine, N,N-diisopropylethylamine, pyridine, 2,6-dimethylpyridine, and the like, in a solvent such as tetrahydrofuran, 1,4-dioxane, acetonitrile, methylene chloride, chloroform, 1,2-dichloroethane, 1,2-dimethoxyethane, and the like, optionally with heating, optionally with microwave irradiation to provide a compound of the formula (276). A compound of the formula (276) is reacted with a compound of the formula (277), a known compound or a compound prepared by known methods, in the presence of a palladium catalyst such as palladium (II) acetate, tetrakis(triphenylphosphine)palladium(0), dichlorobis (triphenylphosphine) palladium(II), palladium on carbon, bis(acetonitrile)dichloropalladium(II), and the like, in the presence of an organophosphine such as 2-dicyclohexylphosphino-2′,6′-dimethoxybiphenyl, 2-dicyclohexylphosphino-2′-(N,N-dimethylamino)biphenyl, 2-dicyclohexylphosphino-2′,4′,6′-triisopropylbiphenyl, 2-di-tert-butylphosphino-2′,4′,6′-triisopropylbiphenyl, (2-biphenyl)dicyclohexyl phosphine, (2-biphenyl)di-tert-butylphosphine, 2-dicyclohexylphosphino-2′,6′-diisopropoxybiphenyl, 2-di-tert-butylphosphino-3,4,5,6-tetramethyl-2′,4′,6′-triisopropyl-1,1′-biphenyl, Sodium 2′-dicyclohexylphosphino-2,6-dimethoxy-1,1′-biphenyl-3-sulfonate, 2-di-tert-butylphosphino-2′-methyl biphenyl, 2-dicyclohexylphosphino-2′-methylbiphenyl, 2′-(di-tert-butylphosphino)-N,N-dimethyl biphenyl-2-amine, 2′-(diphenylphosphino)-N,N′-dimethyl-(1,1′-biphenyl)-2-amine, and the like, in the presence of a base such as sodium carbonate, lithium carbonate, potassium carbonate, cesium carbonate, sodium hydroxide, lithium hydroxide, potassium hydroxide, triethylamine, N,N-diisopropylethylamine, pyridine, 2,6-dimethylpyridine, and the like, in a solvent such as tetrahydrofuran, 1,4-dioxane, acetonitrile, methylene chloride, chloroform, 1,2-dichloroethane, 1,2-dimethoxyethane, and the like, optionally with heating, optionally with microwave irradiation to provide a compound of the formula (278). A compound of the formula (278) is reacted with an acid such as trifluoroacetic acid, hydrochloric acid, sulfuric acid, and the like, in a solvent such as methylene chloride, 1,2-dichloroethane, tetrahydrofuran, 1,4-dioxane, methanol, ethanol, N,N-dimethylformamide, dimethylsulfoxide, N,N-dimethylacetamide, and the like, optionally in the presence of methylphenyl ether, optionally with heating, optionally with microwave irradiation to provide a compound of the formula (279). A compound of the formula (279) is reacted with a compound of the formula (280) in the presence of a base such as triethylamine, N,N-diisopropylethylamine, pyridine, 2,6-dimethylpyridine, and the like, in the presence of a solvent such as N,N-dimethylformamide, dimethylsulfoxide, N,N-dimethylacetamide, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (281). A compound of the formula (281) is reacted with an acid such as trifluoroacetic acid, hydrochloric acid, sulfuric acid, and the like, in a solvent such as methylene chloride, chloroform, 1,2-dichloroethane, tetrahydrofuran, 1,4-dioxane, methanol, ethanol, N,N-dimethylformamide, dimethylsulfoxide, N,N-dimethylacetamide, and the like, optionally in the presence of methylphenyl ether, optionally with heating, optionally with microwave irradiation to provide a compound of the formula (282), A compound of the formula (282) is reacted with a compound of the formula (283), a known compound or a compound prepared by known methods, in the presence of a base such as sodium carbonate, potassium carbonate, lithium carbonate, triethylamine, pyridine, and the like, in a solvent such as N,N-dimethylformamide, dimethylsulfoxide, N,N-dimethylacetamide, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (284).

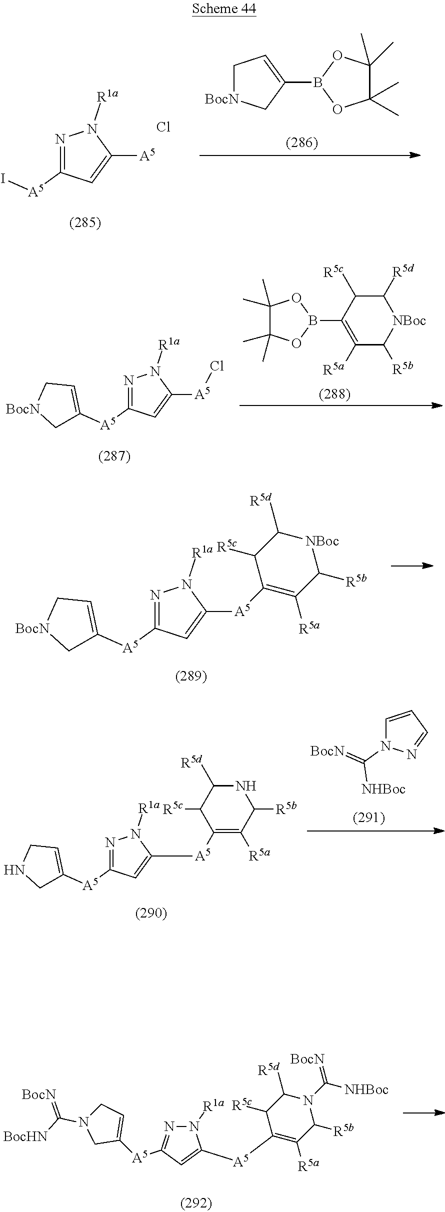

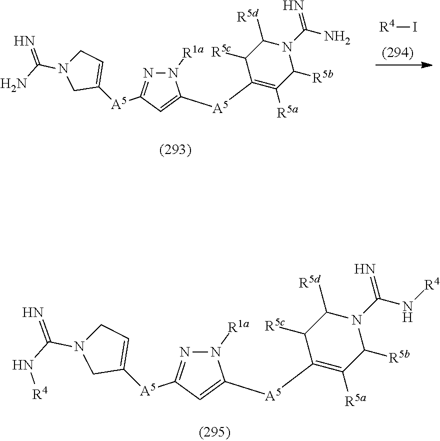

A compound of the formula (285) is reacted with a compound of the formula (286), a known compound or a compound prepared by known methods, in the presence of a palladium catalyst such as palladium (II) acetate, tetrakis(triphenylphosphine)palladium(0), dichlorobis (triphenylphosphine) palladium(II), palladium on carbon, bis(acetonitrile)dichloropalladium(II), and the like, in the presence of an organophosphine such as 2-dicyclohexylphosphino-2′,6′-dimethoxybiphenyl, 2-dicyclohexylphosphino-2′-(N,N-dimethylamino)biphenyl, 2-dicyclohexylphosphino-2′,4′,6′-triisopropylbiphenyl, 2-di-tert-butylphosphino-2′,4′,6′-triisopropylbiphenyl, (2-biphenyl)dicyclohexyl phosphine, (2-biphenyl)di-tert-butylphosphine, 2-dicyclohexylphosphino-2′,6′-diisopropoxybiphenyl, 2-di-tert-butylphosphino-3,4,5,6-tetramethyl-2′,4′,6′-triisopropyl-1,1′-biphenyl, Sodium 2′-dicyclohexylphosphino-2,6-dimethoxy-1,1′-biphenyl-3-sulfonate, 2-di-tert-butylphosphino-2′-methyl biphenyl, 2-dicyclohexylphosphino-2′-methylbiphenyl, 2′-(di-tert-butylphosphino)-N,N-dimethyl biphenyl-2-amine, 2′-(diphenylphosphino)-N,N′-dimethyl-(1,1′-biphenyl)-2-amine, and the like, in the presence of a base such as sodium carbonate, lithium carbonate, potassium carbonate, cesium carbonate, sodium hydroxide, lithium hydroxide, potassium hydroxide, triethylamine, N,N-diisopropylethylamine, pyridine, 2,6-dimethylpyridine, and the like, in a solvent such as tetrahydrofuran, 1,4-dioxane, acetonitrile, methylene chloride, chloroform, 1,2-dichloroethane, 1,2-dimethoxyethane, and the like, optionally with heating, optionally with microwave irradiation to provide a compound of the formula (287). A compound of the formula (287) is reacted with a compound of the formula (288), a known compound or a compound prepared by known methods, in the presence of a palladium catalyst such as palladium (II) acetate, tetrakis(triphenylphosphine)palladium(0), dichlorobis (triphenylphosphine) palladium(II), palladium on carbon, bis(acetonitrile)dichloropalladium(II), and the like, in the presence of an organophosphine such as 2-dicyclohexylphosphino-2′,6′-dimethoxybiphenyl, 2-dicyclohexylphosphino-2′-(N,N-dimethylamino)biphenyl, 2-dicyclohexylphosphino-2′,4′,6′-triisopropylbiphenyl, 2-di-tert-butylphosphino-2′,4′,6′-triisopropylbiphenyl, (2-biphenyl)dicyclohexyl phosphine, (2-biphenyl)di-tert-butylphosphine, 2-dicyclohexylphosphino-2′,6′-diisopropoxybiphenyl, 2-di-tert-butylphosphino-3,4,5,6-tetramethyl-2′,4′,6′-triisopropyl-1,1′-biphenyl, Sodium 2′-dicyclohexylphosphino-2,6-dimethoxy-1,1′-biphenyl-3-sulfonate, 2-di-tert-butylphosphino-2′-methyl biphenyl, 2-dicyclohexylphosphino-2′-methylbiphenyl, 2′-(di-tert-butyl phosphino)-N,N-dimethyl biphenyl-2-amine, 2′-(diphenylphosphino)-N,N′-dimethyl-(1,1′-biphenyl)-2-amine, and the like, in the presence of a base such as sodium carbonate, lithium carbonate, potassium carbonate, cesium carbonate, sodium hydroxide, lithium hydroxide, potassium hydroxide, triethylamine, N,N-diisopropylethylamine, pyridine, 2,6-dimethylpyridine, and the like, in a solvent such as tetrahydrofuran, 1,4-dioxane, acetonitrile, methylene chloride, chloroform, 1,2-dichloroethane, 1,2-dimethoxyethane, and the like, optionally with heating, optionally with microwave irradiation to provide a compound of the formula (289). A compound of the formula (289) is reacted with an acid such as trifluoroacetic acid, hydrochloric acid, sulfuric acid, and the like, in a solvent such as methylene chloride, 1,2-dichloroethane, tetrahydrofuran, 1,4-dioxane, methanol, ethanol, N,N-dimethylformamide, dimethylsulfoxide, N,N-dimethylacetamide, and the like, optionally in the presence of methylphenyl ether, optionally with heating, optionally with microwave irradiation to provide a compound of the formula (290). A compound of the formula (290) is reacted with a compound of the formula (291) in the presence of a base such as triethylamine, N,N-diisopropylethylamine, pyridine, 2,6-dimethylpyridine, and the like, in the presence of a solvent such as N,N-dimethylformamide, dimethylsulfoxide, N,N-dimethylacetamide, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (292). A compound of the formula (292) is reacted with an acid such as trifluoroacetic acid, hydrochloric acid, sulfuric acid, and the like, in a solvent such as methylene chloride, chloroform, 1,2-dichloroethane, tetrahydrofuran, 1,4-dioxane, methanol, ethanol, N,N-dimethylformamide, dimethylsulfoxide, N,N-dimethylacetamide, and the like, optionally in the presence of methylphenyl ether, optionally with heating, optionally with microwave irradiation to provide a compound of the formula (293). A compound of the formula (293) is reacted with a compound of the formula (294), a known compound or a compound prepared by known methods, in the presence of a base such as sodium carbonate, potassium carbonate, lithium carbonate, triethylamine, pyridine, and the like, in a solvent such as N,N-dimethylformamide, dimethylsulfoxide, N,N-dimethylacetamide, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (295).

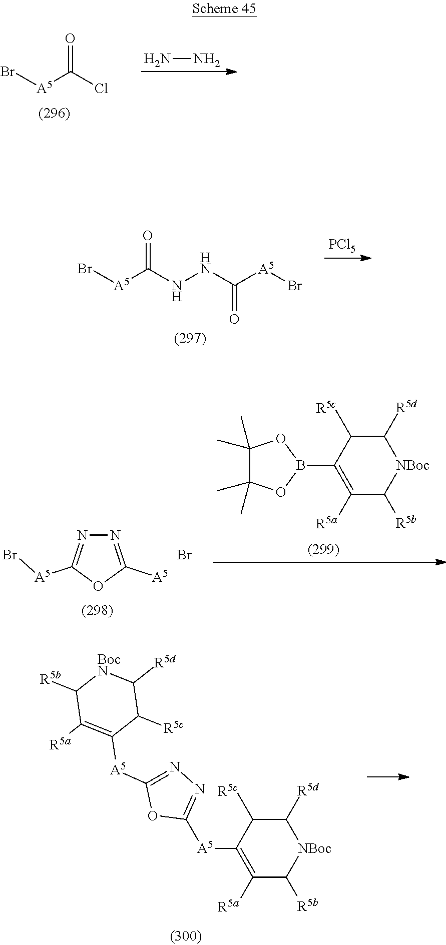

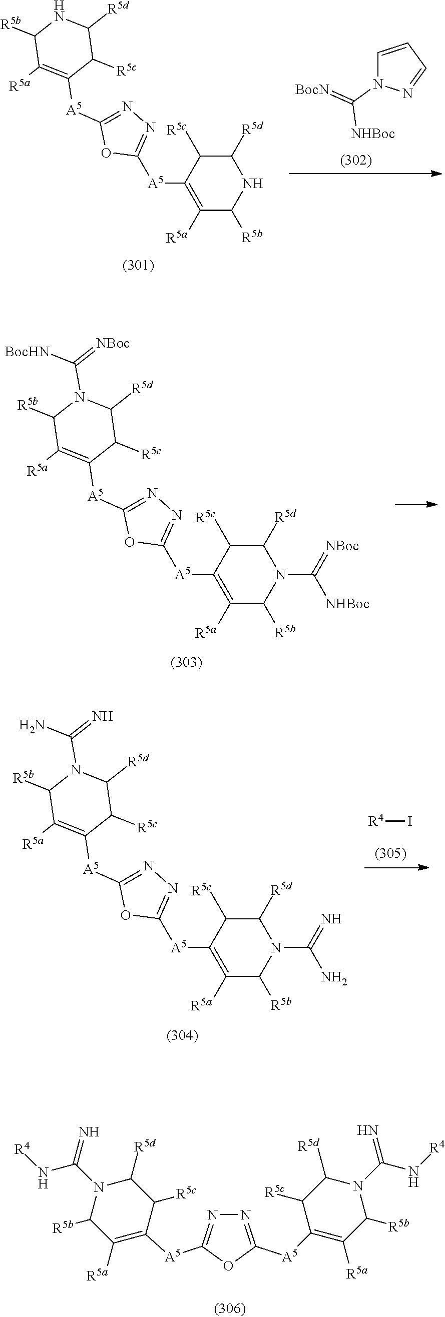

A compound of the formula (296), a known compound or a compound prepared by known methods, is reacted with hydrazine in the presence of a base such as triethylamine, N,N-diisopropylethylamine, pyridine, 2,6-dimethylpyridine, and the like, in the presence of a solvent such as methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (297). A compound of the formula (297) is reacted with phosphorous pentachloride in the presence of a solvent such as benzene, toluene, xylem, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (298). A compound of the formula (298) is reacted with a compound of the formula (299), a known compound or a compound prepared by known methods, in the presence of a palladium catalyst such as palladium (II) acetate, tetrakis(triphenylphosphine)palladium(0), dichlorobis (triphenylphosphine) palladium(II), palladium on carbon, bis(acetonitrile)dichloropalladium(II), and the like, in the presence of an organophosphine such as 2-dicyclohexylphosphino-2′,6′-dimethoxybiphenyl, 2-dicyclohexylphosphino-2′-(N,N-dimethylamino)biphenyl, 2-dicyclohexylphosphino-2′,4′,6′-triisopropylbiphenyl, 2-di-tert-butylphosphino-2′,4′,6′-triisopropylbiphenyl, (2-biphenyl)dicyclohexyl phosphine, (2-biphenyl)di-tert-butylphosphine, 2-dicyclohexylphosphino-2′,6′-diisopropoxybiphenyl, 2-di-tert-butylphosphino-3,4,5,6-tetramethyl-2′,4′,6′-triisopropyl-1,1′-biphenyl, Sodium 2′-dicyclohexylphosphino-2,6-dimethoxy-1,1′-biphenyl-3-sulfonate, 2-di-tert-butylphosphino-2′-methyl biphenyl, 2-dicyclohexylphosphino-2′-methylbiphenyl, 2′-(di-tert-butylphosphino)-N,N-dimethyl biphenyl-2-amine, 2′-(diphenylphosphino)-N,N′-dimethyl-(1,1′-biphenyl)-2-amine, and the like, in the presence of a base such as sodium carbonate, lithium carbonate, potassium carbonate, cesium carbonate, sodium hydroxide, lithium hydroxide, potassium hydroxide, triethylamine, N,N-diisopropylethylamine, pyridine, 2,6-dimethylpyridine, and the like, in a solvent such as tetrahydrofuran, 1,4-dioxane, acetonitrile, methylene chloride, chloroform, 1,2-dichloroethane, 1,2-dimethoxyethane, and the like, optionally with heating, optionally with microwave irradiation to provide a compound of the formula (300). A compound of the formula (300) is reacted with an acid such as trifluoroacetic acid, hydrochloric acid, sulfuric acid, and the like, in a solvent such as methylene chloride, 1,2-dichloroethane, tetrahydrofuran, 1,4-dioxane, methanol, ethanol, N,N-dimethylformamide, dimethylsulfoxide, N,N-dimethylacetamide, and the like, optionally in the presence of methylphenyl ether, optionally with heating, optionally with microwave irradiation to provide a compound of the formula (301). A compound of the formula (301) is reacted with a compound of the formula (302) in the presence of a base such as triethylamine, N,N-diisopropylethylamine, pyridine, 2,6-dimethylpyridine, and the like, in the presence of a solvent such as N,N-dimethylformamide, dimethylsulfoxide, N,N-dimethylacetamide, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (303). A compound of the formula (303) is reacted with an acid such as trifluoroacetic acid, hydrochloric acid, sulfuric acid, and the like, in a solvent such as methylene chloride, chloroform, 1,2-dichloroethane, tetrahydrofuran, 1,4-dioxane, methanol, ethanol, N,N-dimethylformamide, dimethylsulfoxide, N,N-dimethylacetamide, and the like, optionally in the presence of methylphenyl ether, optionally with heating, optionally with microwave irradiation to provide a compound of the formula (304). A compound of the formula (304) is reacted with a compound of the formula (305), a known compound or a compound prepared by known methods, in the presence of a base such as sodium carbonate, potassium carbonate, lithium carbonate, triethylamine, pyridine, and the like, in a solvent such as N,N-dimethylformamide, dimethylsulfoxide, N,N-dimethylacetamide, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (306).

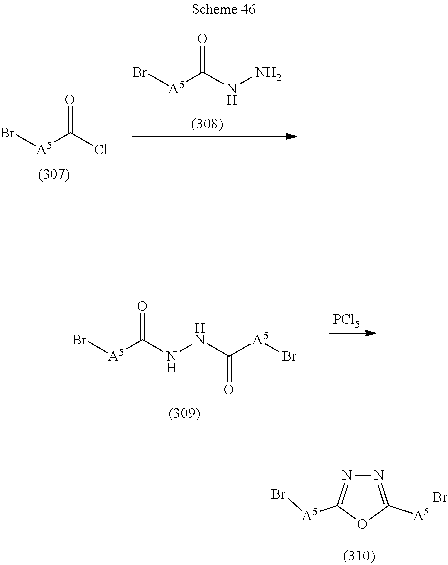

A compound of the formula (307), a known compound or a compound prepared by known methods, is reacted with a compound of the formula (308), a known compound or a compound prepared by known methods, in the presence of a base such as triethylamine, N,N-diisopropylethylamine, pyridine, 2,6-dimethylpyridine, and the like, in the presence of a solvent such as methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (309). A compound of the formula (309) is reacted with phosphorous pentachloride, in the presence of a solvent such as benzene, toluene, xylene, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (310).

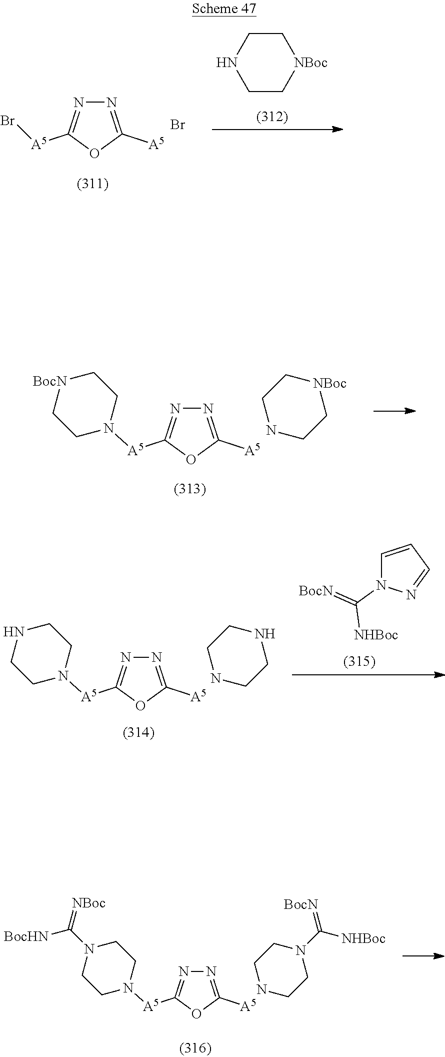

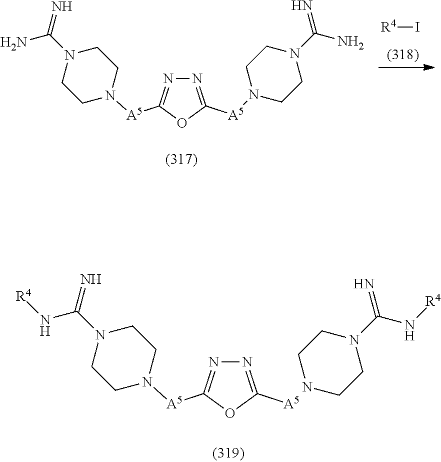

A compound of the formula (311) is reacted with a compound of the formula (312), a known compound or a compound prepared by known methods, in the presence of a palladium catalyst such as palladium (II) acetate, tetrakis(triphenylphosphine)palladium(0), dichlorobis (triphenylphosphine) palladium(II), palladium on carbon, bis(acetonitrile)dichloropalladium(II), and the like, in the presence of an organophosphine such as 2-dicyclohexylphosphino-2′,6′-dimethoxybiphenyl, 2-dicyclohexylphosphino-2′-(N,N-dimethylamino)biphenyl, 2-dicyclohexylphosphino-2′,4′,6′-triisopropylbiphenyl, 2-di-tert-butylphosphino-2′,4′,6′-triisopropylbiphenyl, (2-biphenyl)dicyclohexyl phosphine, (2-biphenyl)di-tert-butylphosphine, 2-dicyclohexylphosphino-2′,6′-diisopropoxybiphenyl, 2-di-tert-butylphosphino-3,4,5,6-tetramethyl-2′,4′,6′-triisopropyl-1,1′-biphenyl, Sodium 2′-dicyclohexylphosphino-2,6-dimethoxy-1,1′-biphenyl-3-sulfonate, 2-di-tert-butylphosphino-2′-methyl biphenyl, 2-dicyclohexyl phosphino-2′-methylbiphenyl, 2′-(di-tert-butyl phosphino)-N,N-dimethyl biphenyl-2-amine, 2′-(diphenylphosphino)-N,N′-dimethyl-(1,1′-biphenyl)-2-amine, and the like, in the presence of a base such as sodium carbonate, lithium carbonate, potassium carbonate, cesium carbonate, sodium hydroxide, lithium hydroxide, potassium hydroxide, triethylamine, N,N-diisopropylethylamine, pyridine, 2,6-dimethylpyridine, and the like, in a solvent such as tetrahydrofuran, 1,4-dioxane, acetonitrile, methylene chloride, chloroform, 1,2-dichloroethane, 1,2-dimethoxyethane, and the like, optionally with heating, optionally with microwave irradiation to provide a compound of the formula (313). A compound of the formula (313) is reacted with an acid such as trifluoroacetic acid, hydrochloric acid, sulfuric acid, and the like, in a solvent such as methylene chloride, 1,2-dichloroethane, tetrahydrofuran, 1,4-dioxane, methanol, ethanol, N,N-dimethylformamide, dimethylsulfoxide, N,N-dimethylacetamide, and the like, optionally in the presence of methylphenyl ether, optionally with heating, optionally with microwave irradiation to provide a compound of the formula (314). A compound of the formula (314) is reacted with a compound of the formula (315) in the presence of a base such as triethylamine, N,N-diisopropylethylamine, pyridine, 2,6-dimethylpyridine, and the like, in the presence of a solvent such as N,N-dimethylformamide, dimethylsulfoxide, N,N-dimethylacetamide, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (316). A compound of the formula (316) is reacted with an acid such as trifluoroacetic acid, hydrochloric acid, sulfuric acid, and the like, in a solvent such as methylene chloride, chloroform, 1,2-dichloroethane, tetrahydrofuran, 1,4-dioxane, methanol, ethanol, N,N-dimethylformamide, dimethylsulfoxide, N,N-dimethylacetamide, and the like, optionally in the presence of methylphenyl ether, optionally with heating, optionally with microwave irradiation to provide a compound of the formula (317). A compound of the formula (317) is reacted with a compound of the formula (318), a known compound or a compound prepared by known methods, in the presence of a base such as sodium carbonate, potassium carbonate, lithium carbonate, triethylamine, pyridine, and the like, in a solvent such as N,N-dimethylformamide, dimethylsulfoxide, N,N-dimethylacetamide, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (319).

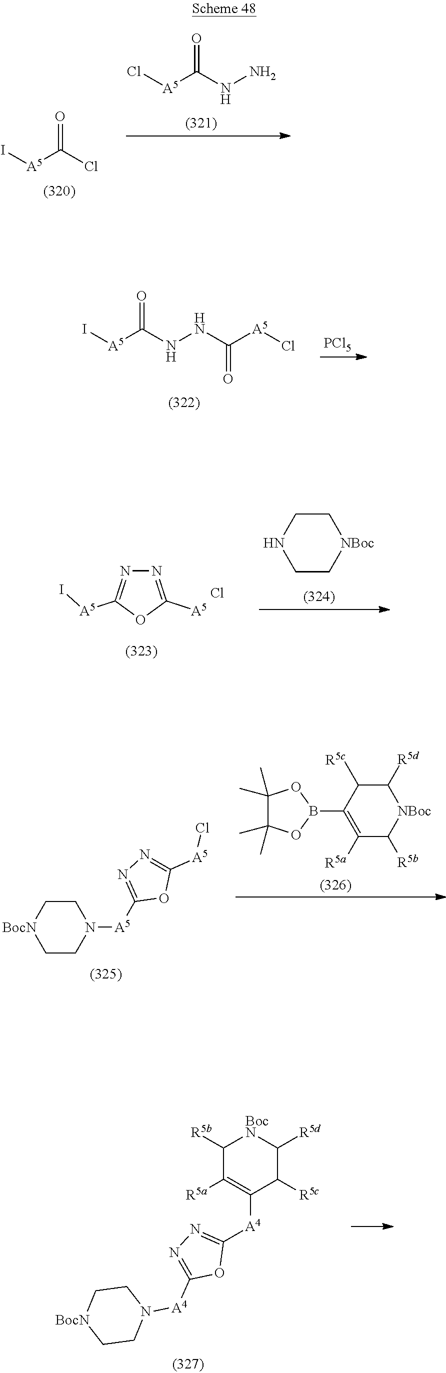

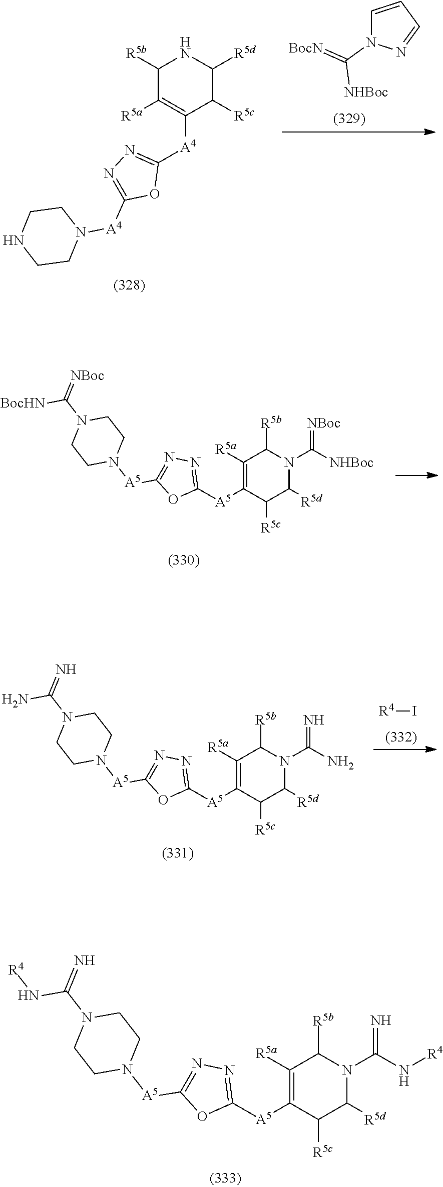

A compound of the formula (320), a known compound or a compound prepared by known methods, is reacted with a compound of the formula (321), a known compound or a compound prepared by known methods, in the presence of a base such as triethylamine, N,N-diisopropylethylamine, pyridine, 2,6-dimethylpyridine, and the like, in the presence of a solvent such as methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (322). A compound of the formula (322) is reacted with phosphorous pentachloride, in the presence of a solvent such as benzene, toluene, xylene, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (323). A compound of the formula (323) is reacted with a compound of the formula (324), a known compound or a compound prepared by known methods, in the presence of a palladium catalyst such as palladium (II) acetate, tetrakis(triphenylphosphine)palladium(0), dichlorobis (triphenylphosphine) palladium(II), palladium on carbon, bis(acetonitrile)dichloropalladium(II), and the like, in the presence of an organophosphine such as 2-dicyclohexylphosphino-2′,6′-dimethoxybiphenyl, 2-dicyclohexylphosphino-2′-(N,N-dimethylamino)biphenyl, 2-di cyclohexyl phosphino-2′,4′,6′-triisopropylbiphenyl, 2-di-tert-butylphosphino-2′,4′,6′-triisopropylbiphenyl, (2-biphenyl)dicyclohexyl phosphine, (2-biphenyl)di-tert-butylphosphine, 2-dicyclohexylphosphino-2′,6′-diisopropoxybiphenyl, 2-di-tert-butylphosphino-3,4,5,6-tetramethyl-2′,4′,6′-triisopropyl-1,1′-biphenyl, Sodium 2′-dicyclohexylphosphino-2,6-dimethoxy-1,1′-biphenyl-3-sulfonate, 2-di-tert-butylphosphino-2′-methyl biphenyl, 2-dicyclohexylphosphino-2′-methylbiphenyl, 2′-(di-tert-butylphosphino)-N,N-dimethyl biphenyl-2-amine, 2′-(diphenylphosphino)-N,N′-dimethyl-(1,1′-biphenyl)-2-amine, and the like, in the presence of a base such as sodium carbonate, lithium carbonate, potassium carbonate, cesium carbonate, sodium hydroxide, lithium hydroxide, potassium hydroxide, triethylamine, N,N-diisopropylethylamine, pyridine, 2,6-dimethylpyridine, and the like, in a solvent such as tetrahydrofuran, 1,4-dioxane, acetonitrile, methylene chloride, chloroform, 1,2-dichloroethane, 1,2-dimethoxyethane, and the like, optionally with heating, optionally with microwave irradiation to provide a compound of the formula (325). A compound of the formula (325) is reacted with a compound of the formula (326), a known compound or a compound prepared by known methods, in the presence of a palladium catalyst such as palladium (II) acetate, tetrakis(triphenylphosphine)palladium(0), dichlorobis (triphenylphosphine) palladium(II), palladium on carbon, bis(acetonitrile)dichloropalladium(II), and the like, in the presence of an organophosphine such as 2-dicyclohexylphosphino-2′,6′-dimethoxybiphenyl, 2-dicyclohexylphosphino-2′-(N,N-dimethylamino)biphenyl, 2-dicyclohexylphosphino-2′,4′,6′-triisopropylbiphenyl, 2-di-tert-butylphosphino-2′,4′,6′-triisopropylbiphenyl, (2-biphenyl)dicyclohexyl phosphine, (2-biphenyl)di-tert-butylphosphine, 2-dicyclohexylphosphino-2′,6′-diisopropoxybiphenyl, 2-di-tert-butylphosphino-3,4,5,6-tetramethyl-2′,4′,6′-triisopropyl-1,1′-biphenyl. Sodium 2′-dicyclohexylphosphino-2,6-dimethoxy-1,1′-biphenyl-3-sulfonate, 2-di-tert-butylphosphino-2′-methyl biphenyl, 2-dicyclohexylphosphino-2′-methylbiphenyl, 2′-(di-tert-butylphosphino)-N,N-dimethyl biphenyl-2-amine, 2′-(diphenylphosphino)-N,N′-dimethyl-(1,1′-biphenyl)-2-amine, and the like, in the presence of a base such as sodium carbonate, lithium carbonate, potassium carbonate, cesium carbonate, sodium hydroxide, lithium hydroxide, potassium hydroxide, triethylamine, N,N-diisopropylethylamine, pyridine, 2,6-dimethylpyridine, and the like, in a solvent such as tetrahydrofuran, 1,4-dioxane, acetonitrile, methylene chloride, chloroform, 1,2-dichloroethane, 1,2-dimethoxyethane, and the like, optionally with heating, optionally with microwave irradiation to provide a compound of the formula (327). A compound of the formula (327) is reacted with an acid such as trifluoroacetic acid, hydrochloric acid, sulfuric acid, and the like, in a solvent such as methylene chloride, 1,2-dichloroethane, tetrahydrofuran, 1,4-dioxane, methanol, ethanol, N,N-dimethylformamide, dimethylsulfoxide, N,N-dimethylacetamide, and the like, optionally in the presence of methylphenyl ether, optionally with heating, optionally with microwave irradiation to provide a compound of the formula (328). A compound of the formula (328) is reacted with a compound of the formula (329) in the presence of a base such as triethylamine, N,N-diisopropylethylamine, pyridine, 2,6-dimethylpyridine, and the like, in the presence of a solvent such as N,N-dimethylformamide, dimethylsulfoxide, N,N-dimethylacetamide, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (330). A compound of the formula (330) is reacted with an acid such as trifluoroacetic acid, hydrochloric acid, sulfuric acid, and the like, in a solvent such as methylene chloride, chloroform, 1,2-dichloroethane, tetrahydrofuran, 1,4-dioxane, methanol, ethanol. N,N-dimethylformamide, dimethylsulfoxide, N,N-dimethylacetamide, and the like, optionally in the presence of methylphenyl ether, optionally with heating, optionally with microwave irradiation to provide a compound of the formula (331). A compound of the formula (331) is reacted with a compound of the formula (332), a known compound or a compound prepared by known methods, in the presence of a base such as sodium carbonate, potassium carbonate, lithium carbonate, triethylamine, pyridine, and the like, in a solvent such as N,N-dimethylformamide, dimethylsulfoxide, N,N-dimethylacetamide, methylene chloride, chloroform, 1,2-dichloroethane, tetrahydrofuran, 1,4-dioxin, acetonitrile, 1,2-dimethoxyethane, and the like, optionally with heating, optionally with microwave irradiation to provide a compound of the formula (333).

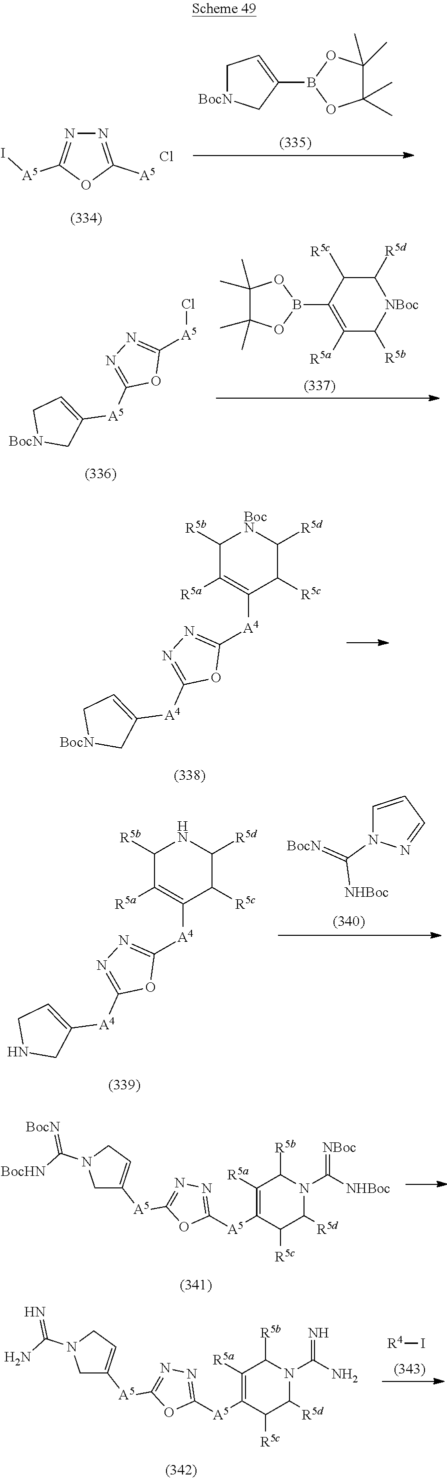

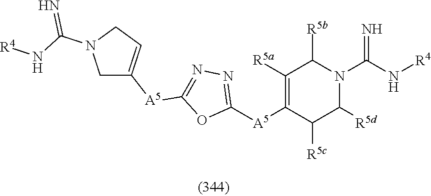

A compound of the formula (334) is reacted with a compound of the formula (335), a known compound or a compound prepared by known methods, in the presence of a palladium catalyst such as palladium (II) acetate, tetrakis(triphenylphosphine)palladium(0), dichlorobis (triphenylphosphine) palladium(II), palladium on carbon, bis(acetonitrile)dichloropalladium(II), and the like, in the presence of an organophosphine such as 2-dicyclohexylphosphino-2′,6′-dimethoxybiphenyl, 2-dicyclohexylphosphino-2′-(1′,N-dimethylamino)biphenyl, 2-dicyclohexylphosphino-2′,4′,6′-tri isopropylbiphenyl, 2-di-tert-butylphosphino-2′,4′,6′-triisopropylbiphenyl, (2-biphenyl)dicyclohexyl phosphine, (2-biphenyl)di-tert-butylphosphine, 2-dicyclohexylphosphino-2′,6′-diisopropoxybiphenyl, 2-di-tert-butylphosphino-3,4,5,6-tetramethyl-2′,4′,6′-triisopropyl-1,1′-biphenyl, Sodium 2′-dicyclohexylphosphino-2,6-dimethoxy-1,1′-biphenyl-3-sulfonate, 2-di-tert-butylphosphino-2′-methyl biphenyl, 2-dicyclohexyl phosphino-2′-methylbiphenyl, 2′-(di-tert-butyl phosphino)-N,N-dimethyl biphenyl-2-amine, 2′-(diphenylphosphino)-N,N′-dimethyl-(1,1′-biphenyl)-2-amine, and the like, in the presence of a base such as sodium carbonate, lithium carbonate, potassium carbonate, cesium carbonate, sodium hydroxide, lithium hydroxide, potassium hydroxide, triethylamine, N,N-diisopropylethylamine, pyridine, 2,6-dimethylpyridine, and the like, in a solvent such as tetrahydrofuran, 1,4-dioxane, acetonitrile, methylene chloride, chloroform, 1,2-dichloroethane, 1,2-dimethoxyethane, and the like, optionally with heating, optionally with microwave irradiation to provide a compound of the formula (336). A compound of the formula (336) is reacted with a compound of the formula (337), a known compound or a compound prepared by known methods, in the presence of a palladium catalyst such as palladium (II) acetate, tetrakis(triphenylphosphine)palladium(0), dichlorobis (triphenylphosphine) palladium(II), palladium on carbon, bis(acetonitrile)dichloropalladium(II), and the like, in the presence of an organophosphine such as 2-dicyclohexylphosphino-2′,6′-dimethoxybiphenyl, 2-dicyclohexylphosphino-2′-(N,N-dimethylamino)biphenyl, 2-dicyclohexylphosphino-2′,4′,6′-triisopropylbiphenyl, 2-di-tert-butylphosphino-2′,4′,6′-triisopropylbiphenyl, (2-biphenyl)dicyclohexyl phosphine, (2-biphenyl)di-tert-butylphosphine, 2-dicyclohexylphosphino-2′,6′-diisopropoxybiphenyl, 2-di-tert-butylphosphino-3,4,5,6-tetramethyl-2′,4′,6′-triisopropyl-1,1′-biphenyl, Sodium 2′-dicyclohexylphosphino-2,6-dimethoxy-1,1′-biphenyl-3-sulfonate, 2-di-tert-butylphosphino-2′-methyl biphenyl, 2-dicyclohexylphosphino-2′-methylbiphenyl, 2′-(di-tert-butylphosphino)-N,N-dimethyl biphenyl-2-amine, 2′-(diphenylphosphino)-N,N′-dimethyl-(1,1′-biphenyl)-2-amine, and the like, in the presence of a base such as sodium carbonate, lithium carbonate, potassium carbonate, cesium carbonate, sodium hydroxide, lithium hydroxide, potassium hydroxide, triethylamine, N,N-diisopropylethylamine, pyridine, 2,6-dimethylpyridine, and the like, in a solvent such as tetrahydrofuran, 1,4-dioxane, acetonitrile, methylene chloride, chloroform, 1,2-dichloroethane, 1,2-dimethoxyethane, and the like, optionally with heating, optionally with microwave irradiation to provide a compound of the formula (338). A compound of the formula (338) is reacted with an acid such as trifluoroacetic acid, hydrochloric acid, sulfuric acid, and the like, in a solvent such as methylene chloride, 1,2-dichloroethane, tetrahydrofuran, 1,4-dioxane, methanol, ethanol, N,N-dimethylformamide, dimethylsulfoxide, N,N-dimethylacetamide, and the like, optionally in the presence of methylphenyl ether, optionally with heating, optionally with microwave irradiation to provide a compound of the formula (339). A compound of the formula (339) is reacted with a compound of the formula (340) in the presence of a base such as triethylamine, N,N-diisopropylethylamine, pyridine, 2,6-dimethylpyridine, and the like, in the presence of a solvent such as N,N-dimethylformamide, dimethylsulfoxide, N,N-dimethylacetamide, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (341). A compound of the formula (341) is reacted with an acid such as trifluoroacetic acid, hydrochloric acid, sulfuric acid, and the like, in a solvent such as methylene chloride, chloroform, 1,2-dichloroethane, tetrahydrofuran, 1,4-dioxane, methanol, ethanol, N,N-dimethylformamide, dimethylsulfoxide, N,N-dimethylacetamide, and the like, optionally in the presence of methylphenyl ether, optionally with heating, optionally with microwave irradiation to provide a compound of the formula (342). A compound of the formula (342) is reacted with a compound of the formula (343), a known compound or a compound prepared by known methods, in the presence of a base such as sodium carbonate, potassium carbonate, lithium carbonate, triethylamine, pyridine, and the like, in a solvent such as N,N-dimethylformamide, dimethylsulfoxide, N,N-dimethylacetamide, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (344).

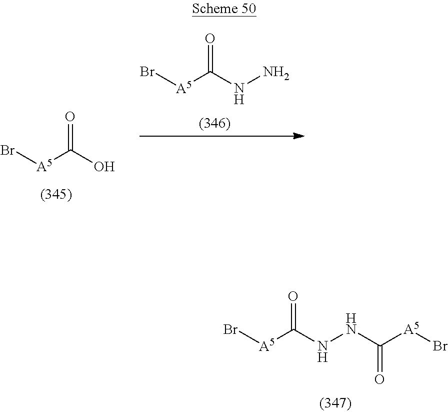

A compound of the formula (345), a known compound or a compound prepared by known methods is reacted with a compound of the formula (346), a known compound or a compound prepared by known methods, in the presence of a coupling agent such as 1-ethyl-3-(3-dimethyl aminopropyl)carbodiimide, [bis(dimethylamino)methylene]-1H-1,2,3-triazolo[4,5-b]pyridinium 3-oxid hexafluorophosphate, O-(benzotriazol-1-yl)-N,N,N,N′-tetramethyluronium hexafluorophosphate, N,N′-dicyclohexylcarbodiimide and the like, optionally in the presence of 1-hydroxy-7-azabenzotriazole, optionally in the presence of hydroxybenzotriazole, in the presence of a base such as triethylamine, N,N-diisopropylethylamine, pyridine, 2,6-dimethylpyridine, and the like, in the presence of a solvent such as hylene chloride, chloroform, 1,2-dichloroethane, tetrahydrofuran, 1,4-dioxane, N,N-dimethylformamide, dimethylsulfoxide, N,N-dimethylacetamide, and the like, optionally with heating, optionally with microwave irradiation to provide a compound of the formula (347).

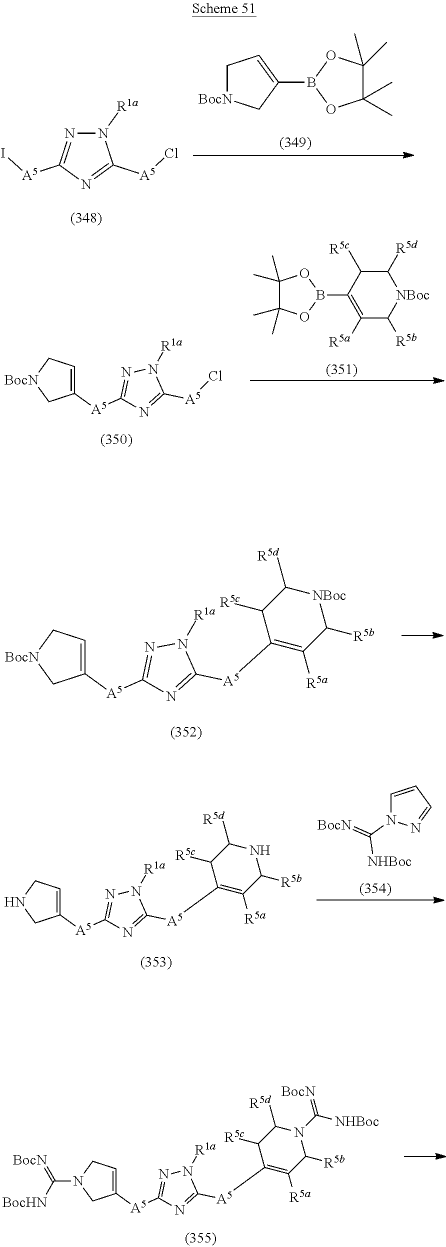

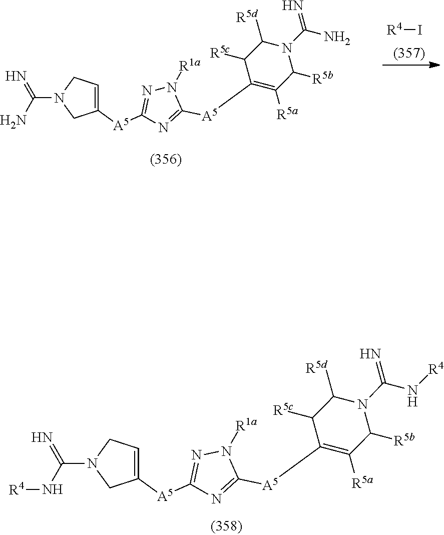

A compound of the formula (348) is reacted with a compound of the formula (349), a known compound or a compound prepared by known methods, in the presence of a palladium catalyst such as palladium (II) acetate, tetrakis(triphenylphosphine)palladium(0), dichlorobis (triphenylphosphine) palladium(II), palladium on carbon, bis(acetonitrile)dichloropalladium(II), and the like, in the presence of an organophosphine such as 2-dicyclohexylphosphino-2′,6′-dimethoxybiphenyl, 2-dicyclohexylphosphino-2′-(N,N-dimethylamino)biphenyl, 2-dicyclohexylphosphino-2′,4′,6′-triisopropylbiphenyl, 2-di-tert-butylphosphino-2′,4′,6′-triisopropylbiphenyl, (2-biphenyl)dicyclohexyl phosphine, (2-biphenyl)di-tert-butylphosphine, 2-dicyclohexylphosphino-2′,6′-diisopropoxybiphenyl, 2-di-tert-butylphosphino-3,4,5,6-tetramethyl-2′,4′,6′-triisopropyl-1,1′-biphenyl, Sodium 2′-dicyclohexylphosphino-2,6-dimethoxy-1,1′-biphenyl-3-sulfonate, 2-di-tert-butylphosphino-2′-methyl biphenyl, 2-dicyclohexylphosphino-2′-methylbiphenyl, 2′-(di-tert-butylphosphino)-N,N-dimethyl biphenyl-2-amine, 2′-(diphenylphosphino)-N,N′-dimethyl-(1,1′-biphenyl)-2-amine, and the like, in the presence of a base such as sodium carbonate, lithium carbonate, potassium carbonate, cesium carbonate, sodium hydroxide, lithium hydroxide, potassium hydroxide, triethylamine, N,N-diisopropylethylamine, pyridine, 2,6-dimethylpyridine, and the like, in a solvent such as tetrahydrofuran, 1,4-dioxane, acetonitrile, methylene chloride, chloroform, 1,2-dichloroethane, 1,2-dimethoxyethane, and the like, optionally with heating, optionally with microwave irradiation to provide a compound of the formula (350). A compound of the formula (350) is reacted with a compound of the formula (351), a known compound or a compound prepared by known methods, in the presence of a palladium catalyst such as palladium (II) acetate, tetrakis(triphenylphosphine)palladium(0), dichlorobis (triphenylphosphine) palladium(II), palladium on carbon, bis(acetonitrile)dichloropalladium(II), and the like, in the presence of an organophosphine such as 2-dicyclohexylphosphino-2′,6′-dimethoxybiphenyl, 2-dicyclohexylphosphino-2′-(N,N-dimethylamino)biphenyl, 2-dicyclohexylphosphino-2′,4′,6′-triisopropylbiphenyl, 2-di-tert-butylphosphino-2′,4′,6′-triisopropylbiphenyl, (2-biphenyl)dicyclohexyl phosphine, (2-biphenyl)di-tert-butylphosphine, 2-dicyclohexylphosphino-2′,6′-diisopropoxybiphenyl, 2-di-tert-butylphosphino-3,4,5,6-tetramethyl-2′,4′,6′-triisopropyl-1,1′-biphenyl, Sodium 2′-dicyclohexylphosphino-2,6-dimethoxy-1,1′-biphenyl-3-sulfonate, 2-di-tert-butylphosphino-2′-methyl biphenyl, 2-dicyclohexylphosphino-2′-methylbiphenyl, 2′-(di-tert-butylphosphino)-N,N-dimethyl biphenyl-2-amine, 2′-(diphenylphosphino)-N,N′-dimethyl-(1,1′-biphenyl)-2-amine, and the like, in the presence of a base such as sodium carbonate, lithium carbonate, potassium carbonate, cesium carbonate, sodium hydroxide, lithium hydroxide, potassium hydroxide, triethylamine, N,N-diisopropylethylamine, pyridine, 2,6-dimethylpyridine, and the like, in a solvent such as tetrahydrofuran, 1,4-dioxane, acetonitrile, methylene chloride, chloroform, 1,2-dichloroethane, 1,2-dimethoxyethane, and the like, optionally with heating, optionally with microwave irradiation to provide a compound of the formula (352). A compound of the formula (352) is reacted with an acid such as trifluoroacetic acid, hydrochloric acid, sulfuric acid, and the like, in a solvent such as methylene chloride, 1,2-dichloroethane, tetrahydrofuran, 1,4-dioxane, methanol, ethanol, N,N-dimethylformamide, dimethylsulfoxide, N,N-dimethylacetamide, and the like, optionally in the presence of methylphenyl ether, optionally with heating, optionally with microwave irradiation to provide a compound of the formula (353). A compound of the formula (353) is reacted with a compound of the formula (354) in the presence of a base such as triethylamine, N,N-diisopropylethylamine, pyridine, 2,6-dimethylpyridine, and the like, in the presence of a solvent such as N,N-dimethylformamide, dimethylsulfoxide, N,N-dimethylacetamide, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (355). A compound of the formula (355) is reacted with an acid such as trifluoroacetic acid, hydrochloric acid, sulfuric acid, and the like, in a solvent such as methylene chloride, chloroform, 1,2-dichloroethane, tetrahydrofuran, 1,4-dioxane, methanol, ethanol. N,N-dimethylformamide, dimethylsulfoxide, N,N-dimethylacetamide, and the like, optionally in the presence of methylphenyl ether, optionally with heating, optionally with microwave irradiation to provide a compound of the formula (356). A compound of the formula (356) is reacted with a compound of the formula (357), a known compound or a compound prepared by known methods, in the presence of a base such as sodium carbonate, potassium carbonate, lithium carbonate, triethylamine, pyridine, and the like, in a solvent such as N,N-dimethylformamide, dimethylsulfoxide, N,N-dimethylacetamide, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (358).

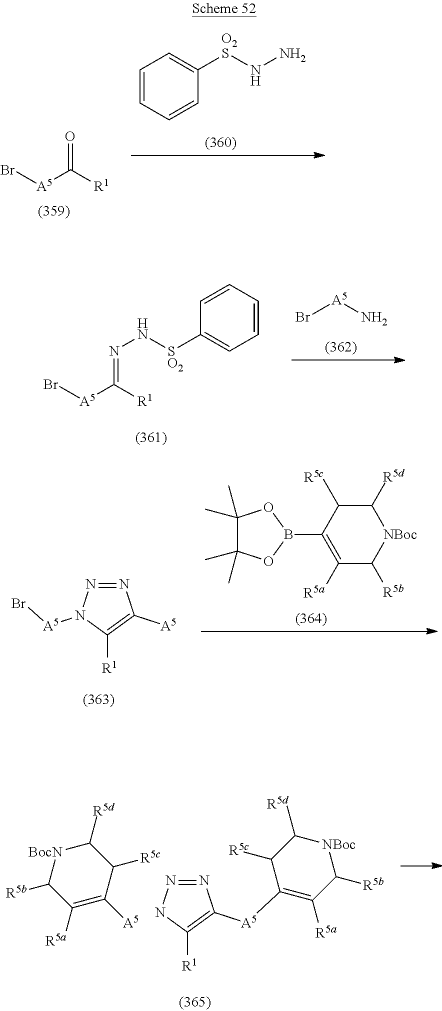

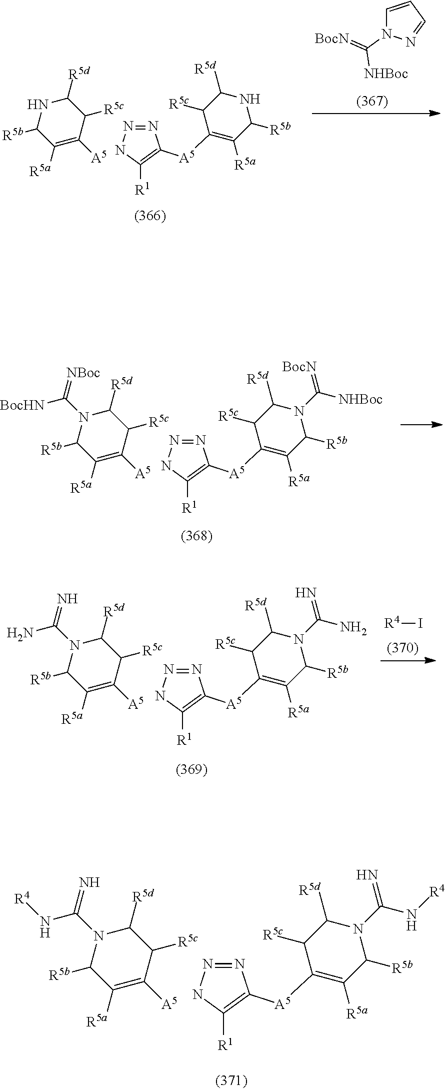

A compound of the formula (359), a known compound or a compound prepared by known methods, is reacted with a compound of the formula (360) in the presence of a solvent such as methanol, ethanol, isopropanol, tetrahydrofuran, 1-4-dioxane, 1,2-dimethoxyethane, methylene chloride, 1,2-dichloroethane, and the like, optionally with heating, optionally with microwave irradiation to provide a compound of the formula (361). A compound of the formula (361) is reacted with a compound of the formula (362), a known compound or a compound prepared by known methods, in the presence of copper acetate, in the presence of pivalic acid, in the presence of a solvent such as benzene, toluene, xylene, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (363). A compound of the formula (363) is reacted with a compound of the formula (364), a known compound or a compound prepared by known methods, in the presence of a palladium catalyst such as palladium (II) acetate, tetrakis(triphenylphosphine)palladium(0), dichlorobis (triphenylphosphine) palladium(II), palladium on carbon, bis(acetonitrile)dichloropalladium(II), and the like, in the presence of an organophosphine such as 2-dicyclohexylphosphino-2′,6′-dimethoxybiphenyl, 2-dicyclohexylphosphino-2′-(N,N-dimethylamino)biphenyl, 2-dicyclohexylphosphino-2′,4′,6′-triisopropylbiphenyl, 2-di-tert-butylphosphino-2′,4′,6′-triisopropylbiphenyl, (2-biphenyl)dicyclohexyl phosphine, (2-biphenyl)di-tert-butylphosphine, 2-dicyclohexylphosphino-2′,6′-diisopropoxybiphenyl, 2-di-tert-butylphosphino-3,4,5,6-tetramethyl-2′,4′,6′-triisopropyl-1,1′-biphenyl, Sodium 2′-dicyclohexylphosphino-2,6-dimethoxy-1,1′-biphenyl-3-sulfonate, 2-di-tert-butylphosphino-2′-methyl biphenyl, 2-dicyclohexylphosphino-2′-methylbiphenyl, 2′-(di-tert-butylphosphino)-N,N-dimethyl biphenyl-2-amine, 2′-(diphenylphosphino)-N,N′-dimethyl-(1,1′-biphenyl)-2-amine, and the like, in the presence of a base such as sodium carbonate, lithium carbonate, potassium carbonate, cesium carbonate, sodium hydroxide, lithium hydroxide, potassium hydroxide, triethylamine, N,N-diisopropylethylamine, pyridine, 2,6-dimethylpyridine, and the like, in a solvent such as tetrahydrofuran, 1,4-dioxane, acetonitrile, methylene chloride, chloroform, 1,2-dichloroethane, 1,2-dimethoxyethane, and the like, optionally with heating, optionally with microwave irradiation to provide a compound of the formula (365). A compound of the formula (365) is reacted with an acid such as trifluoroacetic acid, hydrochloric acid, sulfuric acid, and the like, in a solvent such as methylene chloride, 1,2-dichloroethane, tetrahydrofuran, 1,4-dioxane, methanol, ethanol, N,N-dimethylformamide, dimethylsulfoxide, N,N-dimethylacetamide, and the like, optionally in the presence of methylphenyl ether, optionally with heating, optionally with microwave irradiation to provide a compound of the formula (366). A compound of the formula (366) is reacted with a compound of the formula (367) in the presence of a base such as triethylamine, N,N-diisopropylethylamine, pyridine, 2,6-dimethylpyridine, and the like, in the presence of a solvent such as N,N-dimethylformamide, dimethylsulfoxide, N,N-dimethylacetamide, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (368). A compound of the formula (368) is reacted with an acid such as trifluoroacetic acid, hydrochloric acid, sulfuric acid, and the like, in a solvent such as methylene chloride, chloroform, 1,2-dichloroethane, tetrahydrofuran, 1,4-dioxane, methanol, ethanol. N,N-dimethylformamide, dimethylsulfoxide, N,N-dimethylacetamide, and the like, optionally in the presence of methylphenyl ether, optionally with heating, optionally with microwave irradiation to provide a compound of the formula (369). A compound of the formula (369) is reacted with a compound of the formula (370), a known compound or a compound prepared by known methods, in the presence of a base such as sodium carbonate, potassium carbonate, lithium carbonate, triethylamine, pyridine, and the like, in a solvent such as N,N-dimethylformamide, dimethylsulfoxide, N,N-dimethylacetamide, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (371).

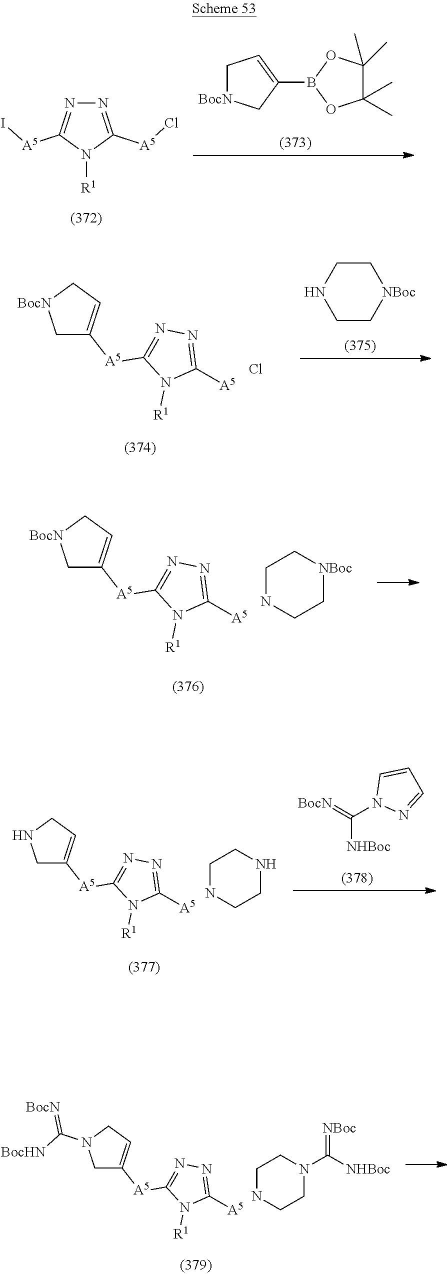

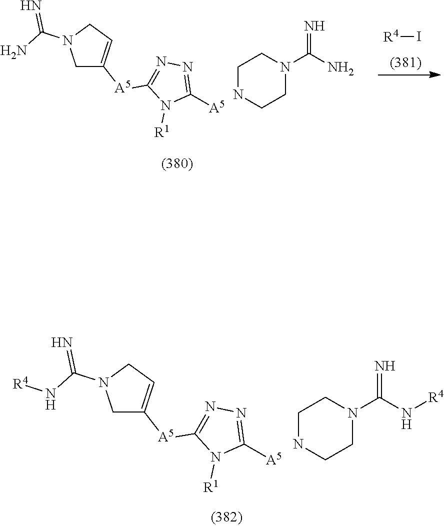

A compound of the formula (372) is reacted with a compound of the formula (373), a known compound or a compound prepared by known methods, in the presence of a palladium catalyst such as palladium (II) acetate, tetrakis(triphenylphosphine)palladium(II), dichlorobis (triphenylphosphine) palladium(II), palladium on carbon, bis(acetonitrile)dichloropalladium(II), and the like, in the presence of an organophosphine such as 2-dicyclohexylphosphino-2′,6′-dimethoxybiphenyl, 2-dicyclohexylphosphino-2′-(N,N-dimethylamino)biphenyl, 2-dicyclohexylphosphino-2′,4′,6′-triisopropylbiphenyl, 2-di-tert-butylphosphino-2′,4′,6′-triisopropylbiphenyl, (2-biphenyl)dicyclohexyl phosphine, (2-biphenyl)di-tert-butylphosphine, 2-dicyclohexylphosphino-2′,6′-diisopropoxybiphenyl, 2-di-tert-butylphosphino-3,4,5,6-tetramethyl-2′,4′,6′-tri isopropyl-1,1′-biphenyl, Sodium 2′-dicyclohexylphosphino-2,6-dimethoxy-1,1′-biphenyl-3-sulfonate, 2-di-tert-butylphosphino-2′-methyl biphenyl, 2-dicyclohexylphosphino-2′-methylbiphenyl, 2′-(di-tert-butylphosphino)-N,N-dimethyl biphenyl-2-amine, 2′-(diphenylphosphino)-N,N′-dimethyl-(1,1′-biphenyl)-2-amine, and the like, in the presence of a base such as sodium carbonate, lithium carbonate, potassium carbonate, cesium carbonate, sodium hydroxide, lithium hydroxide, potassium hydroxide, triethylamine, N,N-diisopropylethylamine, pyridine, 2,6-dimethylpyridine, and the like, in a solvent such as tetrahydrofuran, 1,4-dioxane, acetonitrile, methylene chloride, chloroform, 1,2-dichloroethane, 1,2-dimethoxyethane, and the like, optionally with heating, optionally with microwave irradiation to provide a compound of the formula (374). A compound of the formula (374) is reacted with a compound of the formula (375) in the presence of a base such as sodium t-butoxide, potassium t-butoxide, lithium t-butoxide, sodium methoxide, potassium methoxide, and the like in the presence of a palladium catalyst such as tris(dibenzylideneacetone)dipalladium, palladium (ii) acetate, tetrakis(triphenylphosphine)palladium(0), dichlorobis (triphenylphosphine) palladium(II), palladium on carbon, bis(acetonitrile)dichloropalladium(II), and the like, optionally in the presence of an amino-phosphorous compound such as 2,8,9-Triisobutyl-2,5,8,9-tetraaza-1-phosphabicyclo[3.3.3]undecane, 2,8,9-Trimethyl-2,5,8,9-tetraaza-1-phosphabicyclo[3.3.3]undecane, 2,8,9-Triisopropyl-2,5,8,9-tetraaza-1-phosphabicyclo[3.3.3]undecane and the like, in the presence of a solvent such as toluene, benzene, xylene, tetrahydrofuran, 1,4-dioxane, acetonitrile, N,N-dimethylformamide, dimethylsulfoxide, N,N-dimethylacetamide, and the like, optionally with heating, optionally with microwave irradiation to provide a compound of the formula (376). A compound of the formula (376) is reacted with an acid such as trifluoroacetic acid, hydrochloric acid, sulfuric acid, and the like, in a solvent such as methylene chloride, 1,2-dichloroethane, tetrahydrofuran, 1,4-dioxane, methanol, ethanol, N,N-dimethylformamide, dimethylsulfoxide, N,N-dimethylacetamide, and the like, optionally in the presence of methylphenyl ether, optionally with heating, optionally with microwave irradiation to provide a compound of the formula (377). A compound of the formula (377) is reacted with a compound of the formula (378) in the presence of a base such as triethylamine, N,N-diisopropylethylamine, pyridine, 2,6-dimethylpyridine, and the like, in the presence of a solvent such as N,N-dimethylformamide, dimethylsulfoxide, N,N-dimethylacetamide, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (379). A compound of the formula (379) is reacted with an acid such as trifluoroacetic acid, hydrochloric acid, sulfuric acid, and the like, in a solvent such as methylene chloride, chloroform, 1,2-dichloroethane, tetrahydrofuran, 1,4-dioxane, methanol, ethanol, N,N-dimethylformamide, dimethylsulfoxide, N,N-dimethylacetamide, and the like, optionally in the presence of methylphenyl ether, optionally with heating, optionally with microwave irradiation to provide a compound of the formula (380). A compound of the formula (380) is reacted with a compound of the formula (381), a known compound or a compound prepared by known methods, in the presence of a base such as sodium carbonate, potassium carbonate, lithium carbonate, triethylamine, pyridine, and the like, in a solvent such as N,N-dimethylformamide, dimethylsulfoxide, N,N-dimethylacetamide, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (382).

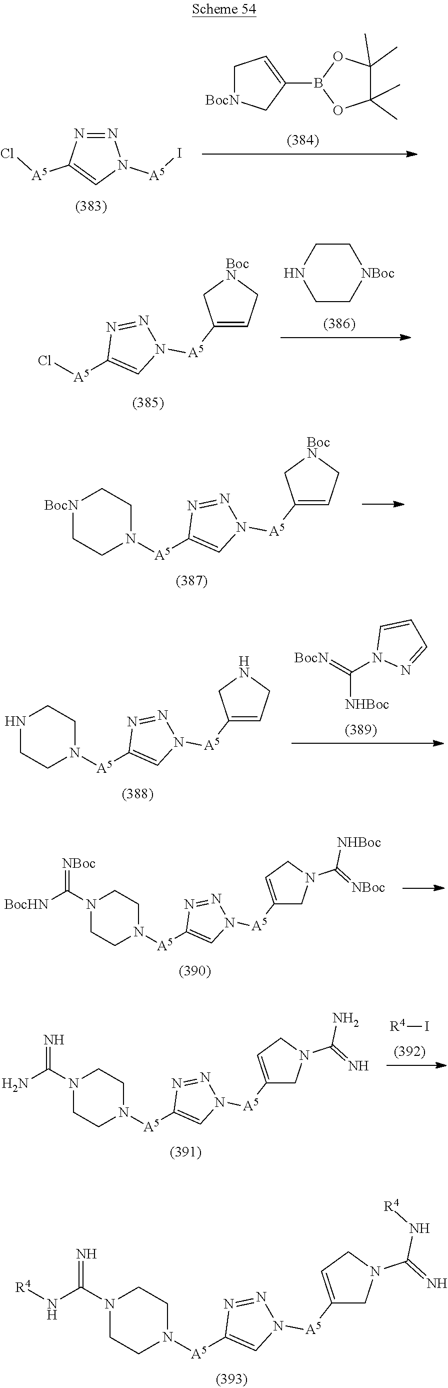

A compound of the formula (383) is reacted with a compound of the formula (384), a known compound or a compound prepared by known methods, in the presence of a palladium catalyst such as palladium (II) acetate, tetrakis(triphenylphosphine)palladium(0), dichlorobis (triphenylphosphine) palladium on carbon, bis(acetonitrile)dichloropalladium(II), and the like, in the presence of an organophosphine such as 2-dicyclohexylphosphino-2′,6′-dimethoxybiphenyl, 2-dicyclohexylphosphino-2′-(N,N-dimethylamino)biphenyl, 2-dicyclohexylphosphino-2′,4′,6′-triisopropylbiphenyl, 2-di-tert-butylphosphino-2′,4′,6′-triisopropylbiphenyl, (2-biphenyl dicyclohexylphosphine, (2-biphenyl)di-tert-butylphosphine, 2-dicyclohexylphosphino-2′,6′-diisopropoxybiphenyl, 2-di-tert-butylphosphino-3,4,5,6-tetramethyl-2′,4′,6′-triisopropyl-1,1′-biphenyl, Sodium 2′-dicyclohexylphosphino-2,6-dimethoxy-1,1-biphenyl-3-sulfonate, 2-di-tert-butylphosphino-2′-methyl biphenyl, 2-dicyclohexylphosphino-2′-methylbiphenyl, 2′-(di-tert-butylphosphino)-N,N-dimethyl biphenyl-2-amine, 2′-(diphenylphosphino)-N,N′-dimethyl-(1,1′-biphenyl)-2-amine, and the like, in the presence of a base such as sodium carbonate, lithium carbonate, potassium carbonate, cesium carbonate, sodium hydroxide, lithium hydroxide, potassium hydroxide, triethylamine, N,N-diisopropylethylamine, pyridine, 2,6-dimethylpyridine, and the like, in a solvent such as tetrahydrofuran, 1,4-dioxane, acetonitrile, methylene chloride, chloroform, 1,2-dichloroethane, 1,2-dimethoxyethane, and the like, optionally with heating, optionally with microwave irradiation to provide a compound of the formula (385). A compound of the formula (385) is reacted with a compound of the formula (386) in the presence of a base such as sodium t-butoxide, potassium t-butoxide, lithium t-butoxide, sodium methoxide, potassium methoxide, and the like in the presence of a palladium catalyst such as tris(dibenzylideneacetone)dipalladium, palladium (II) acetate, tetrakis(triphenylphosphine)palladium(0), dichlorobis (triphenylphosphine) palladium(II), palladium on carbon, bis(acetonitrile)dichloropalladium(II), and the like, optionally in the presence of an amino-phosphorous compound such as 2,8,9-Triisobutyl-2,5,8,9-tetraaza-1-phosphabicyclo[3.3.3]undecane, 2,8,9-Trimethyl-2,5,8,9-tetraaza-1-phosphabicyclo[3.3.3]undecane, 2,8,9-Triisopropyl-2,5,8,9-tetraza-1-phosphabicyclo[3.3.3]undecane and the like, in the presence of a solvent such as toluene, benzene, xylene, tetrahydrofuran, 1,4-dioxane, acetonitrile, N,N-dimethylformamide, dimethylsulfoxide, N,N-dimethylacetamide, and the like, optionally with heating, optionally with microwave irradiation to provide a compound of the formula (387). A compound of the formula (387) is reacted with an acid such as trifluoroacetic acid, hydrochloric acid, sulfuric acid, and the like, in a solvent such as methylene chloride, 1,2-dichloroethane, tetrahydrofuran, 1,4-dioxane, methanol, ethanol, N,N-dimethylformamide, dimethylsulfoxide, N,N-dimethylacetamide, and the like, optionally in the presence of methylphenyl ether, optionally with heating, optionally with microwave irradiation to provide a compound of the formula (388). A compound of the formula (388) is reacted with a compound of the formula (389) in the presence of a base such as triethylamine, N,N-diisopropylethylamine, pyridine, 2,6-dimethylpyridine, and the like, in the presence of a solvent such as N,N-dimethylformamide, dimethylsulfoxide, N,N-dimethylacetamide, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (390). A compound of the formula (390) is reacted with an acid such as trifluoroacetic acid, hydrochloric acid, sulfuric acid, and the like, in a solvent such as methylene chloride, chloroform, 1,2-dichloroethane, tetrahydrofuran, 1,4-dioxane, methanol, ethanol, N,N-dimethylformamide, dimethylsulfoxide, N,N-dimethylacetamide, and the like, optionally in the presence of methylphenyl ether, optionally with heating, optionally with microwave irradiation to provide a compound of the formula (391). A compound of the formula (391) is reacted with a compound of the formula (392), a known compound or a compound prepared by known methods, in the presence of a base such as sodium carbonate, potassium carbonate, lithium carbonate, triethylamine, pyridine, and the like, in a solvent such as N,N-dimethylformamide, dimethylsulfoxide, N,N-dimethylacetamide, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (393).

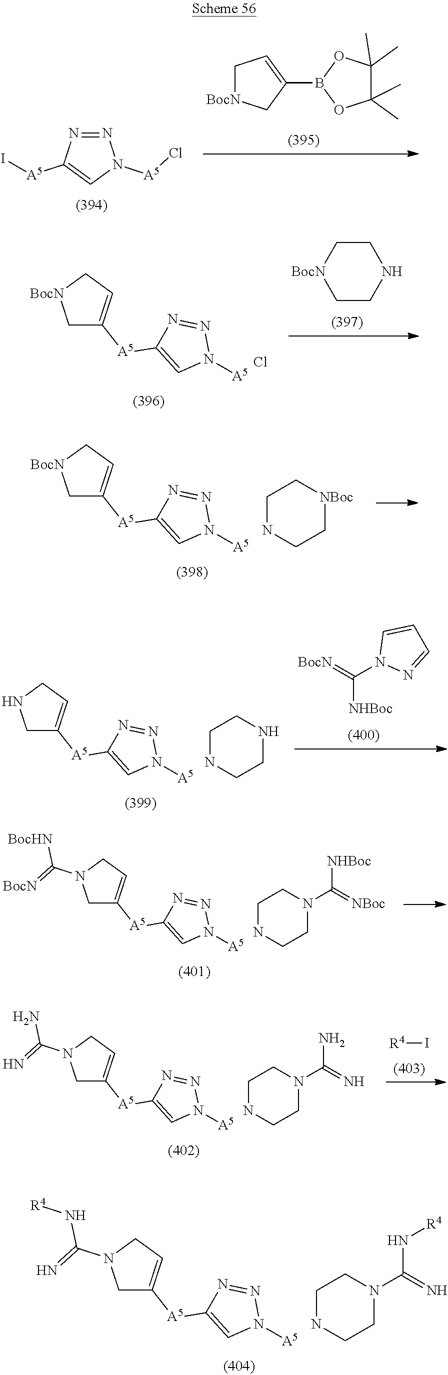

A compound of the formula (394) is reacted with a compound of the formula (395), a known compound or a compound prepared by known methods, in the presence of a palladium catalyst such as palladium (10 acetate, tetrakis(triphenylphosphine)palladium(0), dichlorobis (triphenylphosphine) palladium(II), palladium on carbon, bis(acetonitrile)dichloropalladium(II), and the like, in the presence of an organophosphine such as 2-dicyclohexylphosphino-2′,6′-di methoxybiphenyl, 2-dicyclohexylphosphino-2′-(N,N-dimethylamino)biphenyl, 2-dicyclohexylphosphino-2′,4′,6′-triiso propylbiphenyl, 2-di-tert-butylphosphino-2′,4′,6′-triisopropylbiphenyl, (2-biphenyl)dicyclohexyl phosphine, (2-biphenyl)di-tert-butylphosphine, 2-dicyclohexylphosphino-2′,6′-diisopropoxybiphenyl, 2-di-tert-butylphosphino-3,4,5,6-tetramethyl-2′,4′,6′-triisopropyl-1,1′-biphenyl, Sodium 2′-dicyclohexylphosphino-2,6-dimethoxy-1,1′-biphenyl-3-sulfonate, 2-di-tert-butylphosphino-2′-methyl biphenyl, 2-dicyclohexylphosphino-2′-methylbiphenyl, 2′-(di-tert-butylphosphino)-N,N-dimethyl biphenyl-2-amine, 2′-(diphenylphosphino)-N,N′-dimethyl-(1,1′-biphenyl)-2-amine, and the like, in the presence of a base such as sodium carbonate, lithium carbonate, potassium carbonate, cesium carbonate, sodium hydroxide, lithium hydroxide, potassium hydroxide, triethylamine, N,N-diisopropylethylamine, pyridine, 2,6-dimethylpyridine, and the like, in a solvent such as tetrahydrofuran, 1,4-dioxane, acetonitrile, methylene chloride, chloroform, 1,2-dichloroethane, 1,2-dimethoxyethane, and the like, optionally with heating, optionally with microwave irradiation to provide a compound of the formula (396). A compound of the formula (396) is reacted with a compound of the formula (397) in the presence of a base such as sodium t-butoxide, potassium t-butoxide, lithium t-butoxide, sodium methoxide, potassium methoxide, and the like in the presence of a palladium catalyst such as tris(dibenzylideneacetone)dipalladium, palladium (II) acetate, tetrakis(triphenylphosphine)palladium(0), dichlorobis (triphenylphosphine) palladium(II), palladium on carbon, bis(acetonitrile)dichloropalladium(II), and the like, optionally in the presence of an amino-phosphorous compound such as 2,8,9-Triisobutyl-2,5,8,9-tetraaza-1-phosphabicyclo[3.3.3]undecane, 2,8,9-Trimethyl-2,5,8,9-tetraaza-1-phosphabicyclo[3.3.3]undecane, 2,8,9-Triisopropyl-2,5,8,9-tetraza-1-phosphabicyclo[3.3.3]undecane and the like, in the presence of a solvent such as toluene, benzene, xylene, tetrahydrofuran, 1,4-dioxane, acetonitrile, N,N-dimethylformamide, dimethylsulfoxide, N,N-dimethylacetamide, and the like, optionally with heating, optionally with microwave irradiation to provide a compound of the formula (398). A compound of the formula (398) is reacted with an acid such as trifluoroacetic acid, hydrochloric acid, sulfuric acid, and the like, in a solvent such as methylene chloride, 1,2-dichloroethane, tetrahydrofuran, 1,4-dioxane, methanol, ethanol, N,N-dimethylformamide, dimethylsulfoxide, N,N-dimethylacetamide, and the like, optionally in the presence of methylphenyl ether, optionally with heating, optionally with microwave irradiation to provide a compound of the formula (399). A compound of the formula (399) is reacted with a compound of the formula (400) in the presence of a base such as triethylamine, N,N-diisopropylethylamine, pyridine, 2,6-dimethylpyridine, and the like, in the presence of a solvent such as N,N-dimethylformamide, dimethylsulfoxide, N,N-dimethylacetamide, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (401). A compound of the formula (401) is reacted with an acid such as trifluoroacetic acid, hydrochloric acid, sulfuric acid, and the like, in a solvent such as methylene chloride, chloroform, 1,2-dichloroethane, tetrahydrofuran, 1,4-dioxane, methanol, ethanol, N,N-dimethylformamide, dimethylsulfoxide, N,N-dimethylacetamide, and the like, optionally in the presence of methylphenyl ether, optionally with heating, optionally with microwave irradiation to provide a compound of the formula (402). A compound of the formula (402) is reacted with a compound of the formula (403), a known compound or a compound prepared by known methods, in the presence of a base such as sodium carbonate, potassium carbonate, lithium carbonate, triethylamine, pyridine, and the like, in a solvent such as N,N-dimethylformamide, dimethylsulfoxide, N,N-dimethylacetamide, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (404).

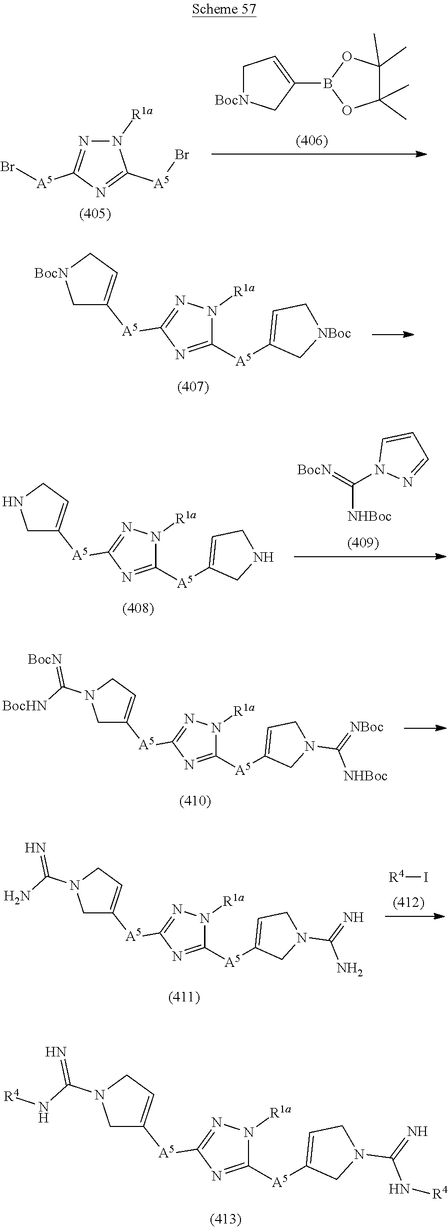

A compound of the formula (405) is reacted with a compound of the formula (406), a known compound or a compound prepared by known methods, in the presence of a palladium catalyst such as palladium (II) acetate, tetrakis(triphenylphosphine)palladium(0), dichlorobis (triphenylphosphine) palladium(II), palladium on carbon, bis(acetonitrile)dichloropalladium(II), and the like, in the presence of an organophosphine such as 2-dicyclohexylphosphino-2′,6′-dimethoxybiphenyl, 2-dicyclohexylphosphino-2′-(N,N-dimethylamino)biphenyl, 2-dicyclohexylphosphino-2′,4′,6′-triisopropylbiphenyl, 2-di-tert-butylphosphino-2′,4′,6′-triisopropylbiphenyl, (2-biphenyl)dicyclohexyl phosphine, (2-biphenyl)di-tert-butylphosphine, 2-dicyclohexylphosphino-2′,6′-diisopropoxybiphenyl, 2-di-tert-butylphosphino-3,4,5,6-tetramethyl-2′,4′,6′-triisopropyl-1,1′-biphenyl. Sodium 2′-dicyclohexylphosphino-2,6-dimethoxy-1,1′-biphenyl-3-sulfonate, 2-di-tert-butylphosphino-2′-methyl biphenyl, 2-dicyclohexylphosphino-2′-methylbiphenyl, 2′-(di-tert-butylphosphino)-N,N-dimethyl biphenyl-2-amine, 2′-(diphenylphosphino)-N,N′-dimethyl-(1,1′-biphenyl)-2-amine, and the like, in the presence of a base such as sodium carbonate, lithium carbonate, potassium carbonate, cesium carbonate, sodium hydroxide, lithium hydroxide, potassium hydroxide, triethylamine, N,N-diisopropylethylamine, pyridine, 2,6-dimethylpyridine, and the like, in a solvent such as tetrahydrofuran, 1,4-dioxane, acetonitrile, methylene chloride, chloroform, 1,2-dichloroethane, 1,2-dimethoxyethane, and the like, optionally with heating, optionally with microwave irradiation to provide a compound of the formula (407). A compound of the formula (407) is reacted with an acid such as trifluoroacetic acid, hydrochloric acid, sulfuric acid, and the like, in a solvent such as methylene chloride, 1,2-dichloroethane, tetrahydrofuran, 1,4-dioxane, methanol, ethanol, N,N-dimethylformamide, dimethylsulfoxide, N,N-dimethylacetamide, and the like, optionally in the presence of methylphenyl ether, optionally with heating, optionally with microwave irradiation to provide a compound of the formula (408). A compound of the formula (408) is reacted with a compound of the formula (409) in the presence of a base such as triethylamine, N,N-diisopropylethylamine, pyridine, 2,6-dimethylpyridine, and the like, in the presence of a solvent such as N,N-dimethylformamide, dimethylsulfoxide, N,N-dimethylacetamide, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (410). A compound of the formula (410) is reacted with an acid such as trifluoroacetic acid, hydrochloric acid, sulfuric acid, and the like, in a solvent such as methylene chloride, chloroform, 1,2-dichloroethane, tetrahydrofuran, 1,4-dioxane, methanol, ethanol, N,N-dimethylformamide, dimethylsulfoxide, N,N-dimethylacetamide, and the like, optionally in the presence of methylphenyl ether, optionally with heating, optionally with microwave irradiation to provide a compound of the formula (411). A compound of the formula (411) is reacted with a compound of the formula (412), a known compound or a compound prepared by known methods, in the presence of a base such as sodium carbonate, potassium carbonate, lithium carbonate, triethylamine, pyridine, and the like, in a solvent such as N,N-dimethylformamide, dimethylsulfoxide, N,N-dimethylacetamide, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (413).

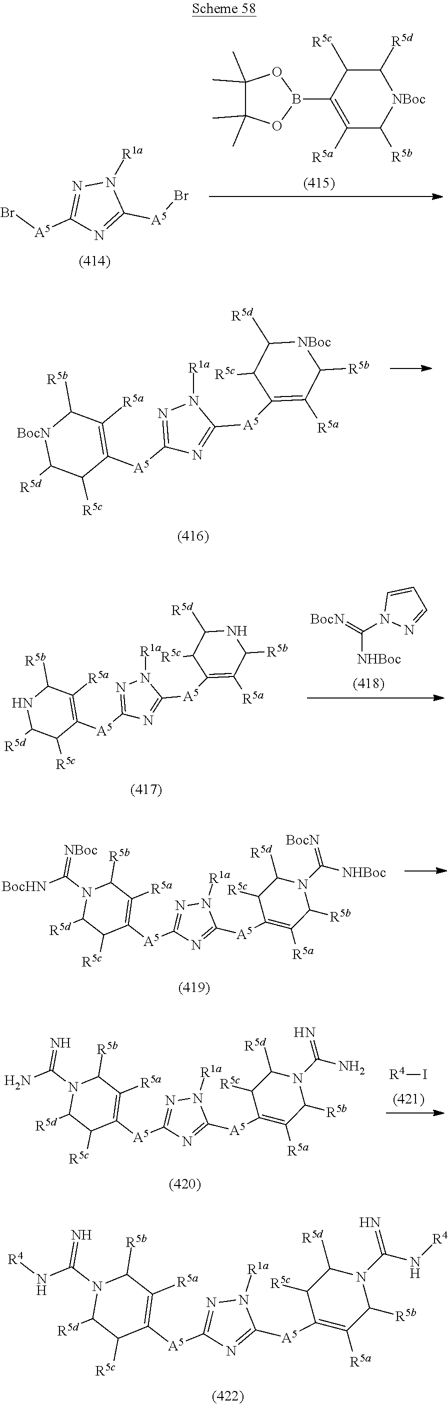

A compound of the formula (414) is reacted with a compound of the formula (415), a known compound or a compound prepared by known methods, in the presence of a palladium catalyst such as palladium (II) acetate, tetrakis(triphenylphosphine)palladium(0), dichlorobis (triphenylphosphine) palladium(II), palladium on carbon, bis(acetonitrile)dichloropalladium(II), and the like, in the presence of an organophosphine such as 2-dicyclohexylphosphino-2′,6-dimethoxybiphenyl, 2-dicyclohexylphosphino-2′-(N,N-dimethylamino)biphenyl, 2-dicyclohexylphosphino-2′,4′,6′-triisopropylbiphenyl, 2-di-tert-butylphosphino-2′,4′,6′-triisopropylbiphenyl, (2-biphenyl)dicyclohexyl phosphine, (2-biphenyl)di-tert-butylphosphine, 2-dicyclohexylphosphino-2′,6′-diisopropoxybiphenyl, 2-di-tert-butylphosphino-3,4,5,6-tetramethyl-2′,4′,6′-triisopropyl-1,1′-biphenyl, Sodium 2′-dicyclohexylphosphino-2,6-dimethoxy-1,1′-biphenyl-3-sulfonate, 2-di-tert-butylphosphino-2′-methyl biphenyl, 2-dicyclohexylphosphino-2-methylbiphenyl, 2′-(di-tert-butylphosphino)-N,N-dimethyl biphenyl-2-amine, 2′-(diphenylphosphino)-N, N-dimethyl-(1,1′-biphenyl)-2-amine, and the like, in the presence of a base such as sodium carbonate, lithium carbonate, potassium carbonate, cesium carbonate, sodium hydroxide, lithium hydroxide, potassium hydroxide, triethylamine, N,N-diisopropylethylamine, pyridine, 2,6-dimethylpyridine, and the like, in a solvent such as tetrahydrofuran, 1,4-dioxane, acetonitrile, methylene chloride, chloroform, 1,2-dichloroethane, 1,2-dimethoxyethane, and the like, optionally with heating, optionally with microwave irradiation to provide a compound of the formula (416). A compound of the formula (41.6) is reacted with an acid such as trifluoroacetic acid, hydrochloric acid, sulfuric acid, and the like, in a solvent such as methylene chloride, 1,2-dichloroethane, tetrahydrofuran, 1,4-dioxane, methanol, ethanol, N,N-dimethylformamide, dimethylsulfoxide, N,N-dimethylacetamide, and the like, optionally in the presence of methylphenyl ether, optionally with heating, optionally with microwave irradiation to provide a compound of the formula (417). A compound of the formula (417) is reacted with a compound of the formula (418) in the presence of a base such as triethylamine, N,N-diisopropylethylamine, pyridine, 2,6-dimethylpyridine, and the like, in the presence of a solvent such as N,N-dimethylformamide, dimethylsulfoxide, N,N-dimethylacetamide, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (419). A compound of the formula (419) is reacted with an acid such as trifluoroacetic acid, hydrochloric acid, sulfuric acid, and the like, in a solvent such as methylene chloride, chloroform, 1,2-dichloroethane, tetrahydrofuran, 1,4-dioxane, methanol, ethanol. N,N-dimethylformamide, dimethylsulfoxide, N,N-dimethylacetamide, and the like, optionally in the presence of methylphenyl ether, optionally with heating, optionally with microwave irradiation to provide a compound of the formula (420). A compound of the formula (420) is reacted with a compound of the formula (421), a known compound or a compound prepared by known methods, in the presence of a base such as sodium carbonate, potassium carbonate, lithium carbonate, triethylamine, pyridine, and the like, in a solvent such as N,N-dimethylformamide, dimethylsulfoxide, N,N-dimethylacetamide, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (422).

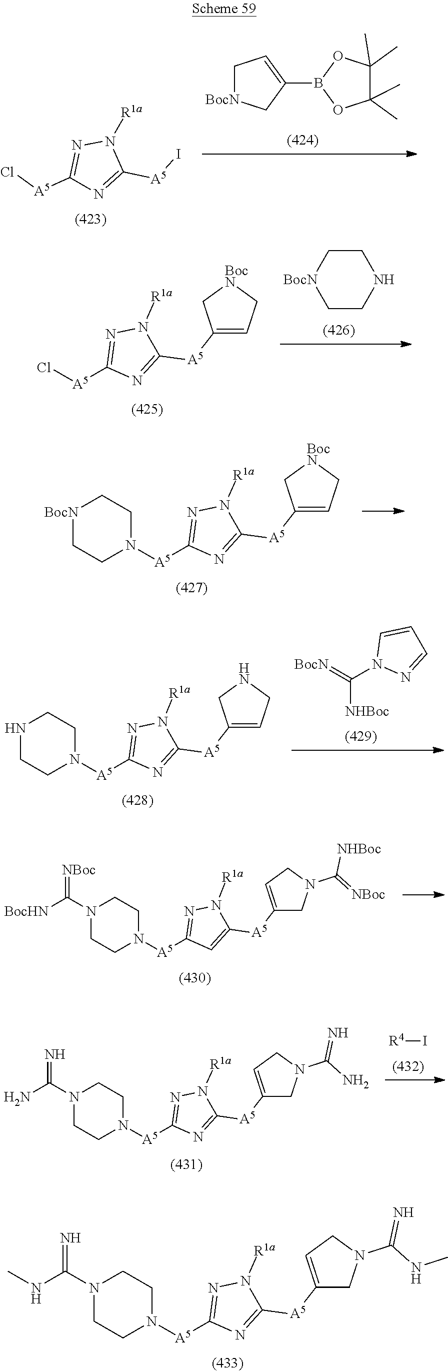

A compound of the formula (423) is reacted with a compound of the formula (424), a known compound or a compound prepared by known methods, in the presence of a palladium catalyst such as palladium (II) acetate, tetrakis(triphenylphosphine)palladium(0), dichlorobis (triphenylphosphine) palladium(II), palladium on carbon, bis(acetonitrile)dichloropalladium(II), and the like, in the presence of an organophosphine such as 2-dicyclohexylphosphino-2′,6′-dimethoxybiphenyl, 2-dicyclohexylphosphino-2′-(N,N-dimethylamino)biphenyl, 2-dicyclohexylphosphino-2′,4′,6′-triisopropylbiphenyl, 2-di-tert-butylphosphino-2′,4′,6′-triisopropylbiphenyl, (2-biphenyl)dicyclohexyl phosphine, (2-biphenyl)di-tert-butylphosphine, 2-dicyclohexylphosphino-2′,6′-diisopropoxybiphenyl, 2-di-tert-butylphosphino-3,4,5,6-tetramethyl-2′,4′,6′-triisopropyl-1,1′-biphenyl, Sodium 2′-dicyclohexylphosphino-2,6-dimethoxy-1,1′-biphenyl-3-sulfonate, 2-di-tert-butylphosphino-2′-methyl biphenyl, 2-dicyclohexylphosphino-2′-methylbiphenyl, 2′-(di-tert-butylphosphino)-N,N-dimethyl biphenyl-2-amine, 2′-(diphenylphosphino)-N,N′-dimethyl-(1,1′-biphenyl)-2-amine, and the like, in the presence of a base such as sodium carbonate, lithium carbonate, potassium carbonate, cesium carbonate, sodium hydroxide, lithium hydroxide, potassium hydroxide, triethylamine, N,N-diisopropylethylamine, pyridine, 2,6-dimethylpyridine, and the like, in a solvent such as tetrahydrofuran, 1,4-dioxane, acetonitrile, methylene chloride, chloroform, 1,2-dichloroethane, 1,2-dimethoxyethane, and the like, optionally with heating, optionally with microwave irradiation to provide a compound of the formula (425). A compound of the formula (425) is reacted with a compound of the formula (426) in the presence of a base such as sodium t-butoxide, potassium t-butoxide, lithium t-butoxide, sodium methoxide, potassium methoxide, and the like in the presence of a palladium catalyst such as tris(dibenzylideneacetone)dipalladium, palladium (II) acetate, tetrakis(triphenylphosphine)palladium(0), dichlorobis (triphenylphosphine) palladium(II), palladium on carbon, bis(acetonitrile)dichloropalladium(II), and the like, optionally in the presence of an amino-phosphorous compound such as 2,8,9-Triisobutyl-2,5,8,9-tetraaza-1-phosphabicyclo[3.3.3]undecane, 2,8,9-Trimethyl-2,5,8,9-tetraaza-1-phosphabicyclo[3.3.3]undecane, 2,8,9-Triisopropyl-2,5,8,9-tetraza-1-phosphabicyclo[3.3.3]undecane and the like, in the presence of a solvent such as toluene, benzene, xylene, tetrahydrofuran, 1,4-dioxane, acetonitrile, N,N-dimethylformamide, dimethylsulfoxide, N,N-dimethylacetamide, and the like, optionally with heating, optionally with microwave irradiation to provide a compound of the formula (427). A compound of the formula (427) is reacted with an acid such as trifluoroacetic acid, hydrochloric acid, sulfuric acid, and the like, in a solvent such as methylene chloride, 1,2-dichloroethane, tetrahydrofuran, 1,4-dioxane, methanol, ethanol, N,N-dimethylformamide, dimethylsulfoxide, N,N-dimethylacetamide, and the like, optionally in the presence of methylphenyl ether, optionally with heating, optionally with microwave irradiation to provide a compound of the formula (428). A compound of the formula (428) is reacted with a compound of the formula (429) in the presence of a base such as triethylamine, N,N-diisopropylethylamine, pyridine, 2,6-dimethylpyridine, and the like, in the presence of a solvent such as N,N-dimethylformamide, dimethylsulfoxide, N,N-dimethylacetamide, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (430). A compound of the formula (430) is reacted with an acid such as trifluoroacetic acid, hydrochloric acid, sulfuric acid, and the like, in a solvent such as methylene chloride, chloroform, 1,2-dichloroethane, tetrahydrofuran, 1,4-dioxane, methanol, ethanol, N,N-dimethylformamide, dimethylsulfoxide, N,N-dimethylacetamide, and the like, optionally in the presence of methylphenyl ether, optionally with heating, optionally with microwave irradiation to provide a compound of the formula (431). A compound of the formula (431) is reacted with a compound of the formula (432), a known compound or a compound prepared by known methods, in the presence of a base such as sodium carbonate, potassium carbonate, lithium carbonate, triethylamine, pyridine, and the like, in a solvent such as N,N-dimethylformamide, dimethylsulfoxide, N,N-dimethylacetamide, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (433).

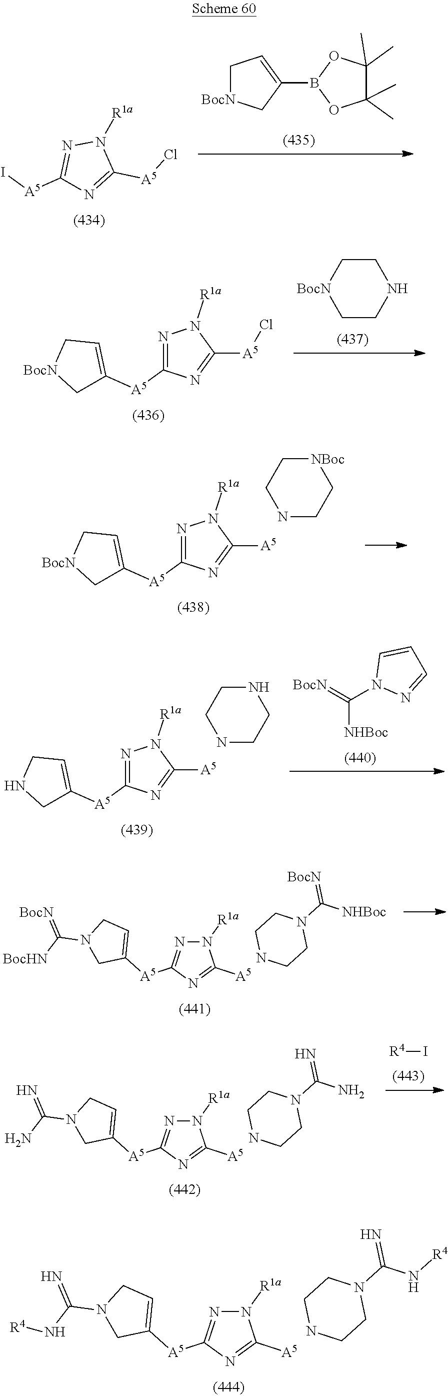

A compound of the formula (434) is reacted with a compound of the formula (435), a known compound or a compound prepared by known methods, in the presence of a palladium catalyst such as palladium (II) acetate, tetrakis(triphenylphosphine)palladium(0), dichlorobis (triphenylphosphine) palladium(II), palladium on carbon, bis(acetonitrile)dichloropalladium(II), and the like, in the presence of an organophosphine such as 2-dicyclohexylphosphino-2′,6′-dimethoxybiphenyl, 2-dicyclohexylphosphino-2′-(N,N-dimethylamino)biphenyl, 2-dicyclohexylphosphino-2′,4′,6′-triisopropylbiphenyl, 2-di-tert-butylphosphino-2′,4′,6′-triisopropylbiphenyl, (2-biphenyl)dicyclohexyl phosphine, (2-biphenyl)di-tert-butylphosphine, 2-dicyclohexylphosphino-2′,6′-diisopropoxybiphenyl, 2-di-tert-butylphosphino-3,4,5,6-tetramethyl-2′,4′,6′-triisopropyl-1,1′-biphenyl, Sodium 2′-dicyclohexylphosphino-2,6-dimethoxy-1,1′-biphenyl-3-sulfonate, 2-di-tert-butylphosphino-2′-methyl biphenyl, 2-dicyclohexylphosphino-2′-methylbiphenyl, 2′-(di-tert-butylphosphino)-N,N-dimethyl biphenyl-2-amine, 2′-(diphenylphosphino)-N,N′-dimethyl-(1,1′-biphenyl)-2-amine, and the like, in the presence of a base such as sodium carbonate, lithium carbonate, potassium carbonate, cesium carbonate, sodium hydroxide, lithium hydroxide, potassium hydroxide, triethylamine, N,N-diisopropylethylamine, pyridine, 2,6-dimethylpyridine, and the like, in a solvent such as tetrahydrofuran, 1,4-dioxane, acetonitrile, methylene chloride, chloroform, 1,2-dichloroethane, 1,2-dimethoxyethane, and the like, optionally with heating, optionally with microwave irradiation to provide a compound of the formula (436). A compound of the formula (436) is reacted with a compound of the formula (437) in the presence of a base such as sodium t-butoxide, potassium t-butoxide, lithium t-butoxide, sodium methoxide, potassium methoxide, and the like in the presence of a palladium catalyst such as tris(dibenzylideneacetone)dipalladium, palladium (II) acetate, tetrakis(triphenylphosphine)palladium(0), dichlorobis (triphenylphosphine) palladium(II), palladium on carbon, bis(acetonitrile)dichloropalladium(II), and the like, optionally in the presence of an amino-phosphorous compound such as 2,8,9-Triisobutyl-2,5,8,9-tetraaza-1-phosphabicyclo[3.3.3]undecane, 2,8,9-Trimethyl-2,5,8,9-tetraaza-1-phosphabicyclo[3.3.3]undecane, 2,8,9-Triisopropyl-2,5,8,9-tetraza-1-phosphabicyclo[3.3.3]undecane and the like, in the presence of a solvent such as toluene, benzene, xylene, tetrahydrofuran, 1,4-dioxane, acetonitrile, N,N-dimethylformamide, dimethylsulfoxide, N,N-dimethylacetamide, and the like, optionally with heating, optionally with microwave irradiation to provide a compound of the formula (438). A compound of the formula (438) is reacted with an acid such as trifluoroacetic acid, hydrochloric acid, sulfuric acid, and the like, in a solvent such as methylene chloride, 1,2-dichloroethane, tetrahydrofuran, 1,4-dioxane, methanol, ethanol, N,N-dimethylformamide, dimethylsulfoxide, N,N-dimethylacetamide, and the like, optionally in the presence of methylphenyl ether, optionally with heating, optionally with microwave irradiation to provide a compound of the formula (439). A compound of the formula (439) is reacted with a compound of the formula (440) in the presence of a base such as triethylamine, N,N-diisopropylethylamine, pyridine, 2,6-dimethylpyridine, and the like, in the presence of a solvent such as N,N-dimethylformamide, dimethylsulfoxide, N,N-dimethylacetamide, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (441). A compound of the formula (441) is reacted with an acid such as trifluoroacetic acid, hydrochloric acid, sulfuric acid, and the like, in a solvent such as methylene chloride, chloroform, 1,2-dichloroethane, tetrahydrofuran, 1,4-dioxane, methanol, ethanol, N,N-dimethylformamide, dimethylsulfoxide, N,N-dimethylacetamide, and the like, optionally in the presence of methylphenyl ether, optionally with heating, optionally with microwave irradiation to provide a compound of the formula (442). A compound of the formula (442) is reacted with a compound of the formula (443), a known compound or a compound prepared by known methods, in the presence of a base such as sodium carbonate, potassium carbonate, lithium carbonate, triethylamine, pyridine, and the like, in a solvent such as N,N-dimethylformamide, dimethylsulfoxide, N,N-dimethylacetamide, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (444).

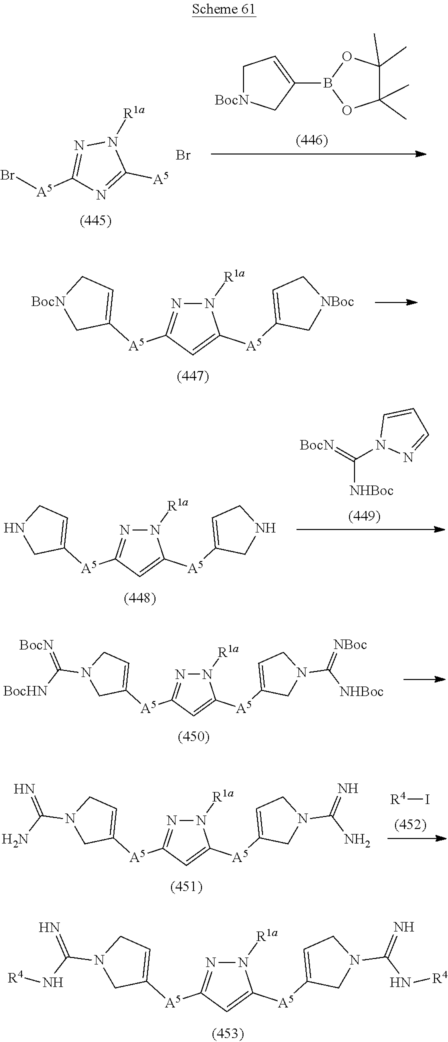

A compound of the formula (445) is reacted with a compound of the formula (446), a known compound or a compound prepared by known methods, in the presence of a palladium catalyst such as palladium (ii) acetate, tetrakis(triphenylphosphine)palladium(0), dichlorobis (triphenylphosphine) palladium(II), palladium on carbon, bis(acetonitrile)dichloropalladium(II), and the like, in the presence of an organophosphine such as 2-dicyclohexylphosphino-2′,6′-dimethoxybiphenyl, 2-dicyclohexylphosphino-2′-(N,N-dimethylamino)biphenyl, 2-dicyclohexylphosphino-2′,4′,6′-triisopropylbiphenyl, 2-di-tert-butylphosphino-2′,4′,6′-triisopropylbiphenyl, (2-biphenyl)dicyclohexyl phosphine, (2-biphenyl)di-tert-butylphosphine, 2-dicyclohexylphosphino-2′,6′-diisopropoxybiphenyl, 2-di-tert-butylphosphino-3,4,5,6-tetramethyl-2′,4′,6′-triisopropyl-1,1′-biphenyl, Sodium 2′-dicyclohexylphosphino-2,6-dimethoxy-1,1′-biphenyl-3-sulfonate, 2-di-tert-butylphosphino-2′-methyl biphenyl, 2-dicyclohexylphosphino-2′-methyl biphenyl, 2′-(di-tert-butylphosphino)-N,N-dimethyl biphenyl-2-amine, 2′-(diphenylphosphino)-N,N′-dimethyl-(1,1′-biphenyl)-2-amine, and the like, in the presence of a base such as sodium carbonate, lithium carbonate, potassium carbonate, cesium carbonate, sodium hydroxide, lithium hydroxide, potassium hydroxide, triethylamine, N,N-diisopropylethylamine, pyridine, 2,6-dimethylpyridine, and the like, in a solvent such as tetrahydrofuran, 1,4-dioxane, acetonitrile, methylene chloride, chloroform, 1,2-dichloroethane, 1,2-dimethoxyethane, and the like, optionally with heating, optionally with microwave irradiation to provide a compound of the formula (447). A compound of the formula (447) is reacted with an acid such as trifluoroacetic acid, hydrochloric acid, sulfuric acid, and the like, in a solvent such as methylene chloride, 1,2-dichloroethane, tetrahydrofuran, 1,4-dioxane, methanol, ethanol, N,N-dimethylformamide, dimethylsulfoxide, N,N-dimethylacetamide, and the like, optionally in the presence of methylphenyl ether, optionally with heating, optionally with microwave irradiation to provide a compound of the formula (448). A compound of the formula (448) is reacted with a compound of the formula (449) in the presence of a base such as triethylamine, N,N-diisopropylethylamine, pyridine, 2,6-dimethylpyridine, and the like, in the presence of a solvent such as N,N-dimethylformamide, dimethylsulfoxide, N,N-dimethylacetamide, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (450). A compound of the formula (450) is reacted with an acid such as trifluoroacetic acid, hydrochloric acid, sulfuric acid, and the like, in a solvent such as methyl ene chloride, chloroform, 1,2-dichloromethane, tetrahydrofuran, 1,4-dioxane, methanol, ethanol, N,N-dimethylformamide, dimethylsulfoxide, N,N-dimethylacetamide, and the like, optionally in the presence of methylphenyl ether, optionally with heating, optionally with microwave irradiation to provide a compound of the formula (451). A compound of the formula (451) is reacted with a compound of the formula (452), a known compound or a compound prepared by known methods, in the presence of a base such as sodium carbonate, potassium carbonate, lithium carbonate, triethylamine, pyridine, and the like, in a solvent such as N,N-dimethylformamide, dimethylsulfoxide, N,N-dimethylacetamide, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (453).

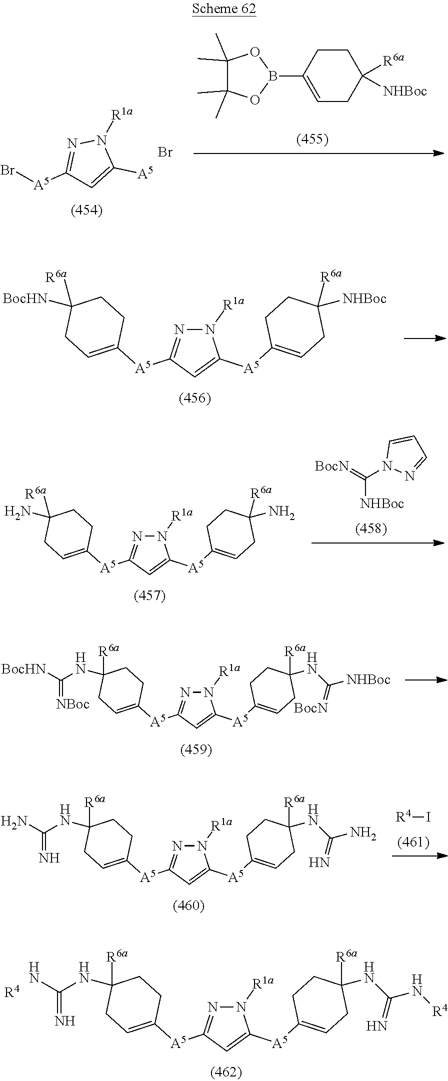

A compound of the formula (454) is reacted with a compound of the formula (455), a known compound or a compound prepared by known methods, in the presence of a palladium catalyst such as palladium (II) acetate, tetrakis(triphenylphosphine)palladium(0), dichlorobis (triphenylphosphine) palladium(II), palladium on carbon, bis(acetonitrile)dichloropalladium(II), and the like, in the presence of an organophosphine such as 2-dicyclohexylphosphino-2′,6′-dimethoxybiphenyl, 2-dicyclohexylphosphino-2′-(N,N-dimethylamino)biphenyl, 2-dicyclohexylphosphino-2′,4′,6′-triisopropylbiphenyl, 2-di-tert-butylphosphino-2′,4′,6′-triisopropylbiphenyl, (2-biphenyl)dicyclohexyl phosphine, (2-biphenyl)di-tert-butylphosphine, 2-dicyclohexylphosphino-2′,6′-diisopropoxybiphenyl, 2-di-tert-butylphosphino-3,4,5,6-tetramethyl-2′,4′,6′-triisopropyl-1,1′-biphenyl, Sodium 2′-dicyclohexylphosphino-2,6-dimethoxy-1,1′-biphenyl-3-sulfonate, 2-di-tert-butylphosphino-2′-methyl biphenyl, 2-dicyclohexylphosphino-2′-methylbiphenyl, 2′-(di-tert-butylphosphino)-N,N-dimethyl biphenyl-2-amine, 2′-(diphenylphosphino)-N,N′-dimethyl-(1,1′-biphenyl)-2-amine, and the like, in the presence of a base such as sodium carbonate, lithium carbonate, potassium carbonate, cesium carbonate, sodium hydroxide, lithium hydroxide, potassium hydroxide, triethylamine, N,N-diisopropylethylamine, pyridine, 2,6-dimethylpyridine, and the like, in a solvent such as tetrahydrofuran, 1,4-dioxane, acetonitrile, methylene chloride, chloroform, 1,2-dichloroethane, 1,2-dimethoxyethane, and the like, optionally with heating, optionally with microwave irradiation to provide a compound of the formula (456). A compound of the formula (456) is reacted with an acid such as trifluoroacetic acid, hydrochloric acid, sulfuric acid, and the like, in a solvent such as methylene chloride, 1,2-dichloroethane, tetrahydrofuran, 1,4-dioxane, methanol, ethanol, N,N-dimethylformamide, dimethylsulfoxide, N,N-dimethylacetamide, and the like, optionally in the presence of methylphenyl ether, optionally with heating, optionally with microwave irradiation to provide a compound of the formula (457). A compound of the formula (457) is reacted with a compound of the formula (458) in the presence of a base such as triethylamine, N,N-diisopropylethylamine, pyridine, 2,6-dimethylpyridine, and the like, in the presence of a solvent such as N,N-dimethylformamide, dimethylsulfoxide, N,N-dimethylacetamide, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (459). A compound of the formula (459) is reacted with an acid such as trifluoroacetic acid, hydrochloric acid, sulfuric acid, and the like, in a solvent such as methylene chloride, chloroform, 1,2-dichloroethane, tetrahydrofuran, 1,4-dioxane, methanol, ethanol, N,N-dimethylformamide, dimethylsulfoxide, N,N-dimethylacetamide, and the like, optionally in the presence of methylphenyl ether, optionally with heating, optionally with microwave irradiation to provide a compound of the formula (460). A compound of the formula (460) is reacted with a compound of the formula (461), a known compound or a compound prepared by known methods, in the presence of a base such as sodium carbonate, potassium carbonate, lithium carbonate, triethylamine, pyridine, and the like, in a solvent such as N,N-dimethylformamide, dimethylsulfoxide, N,N-dimethylacetamide, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (462).

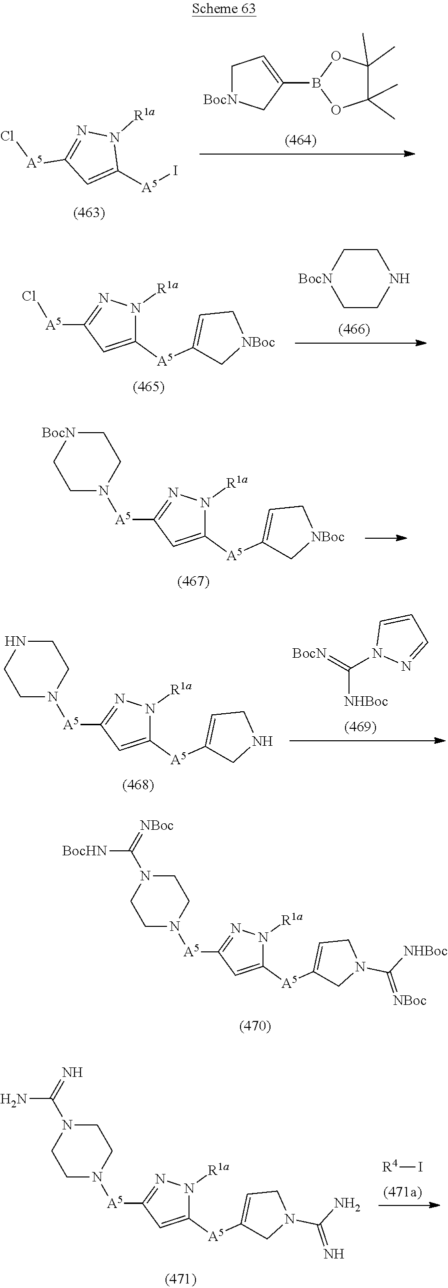

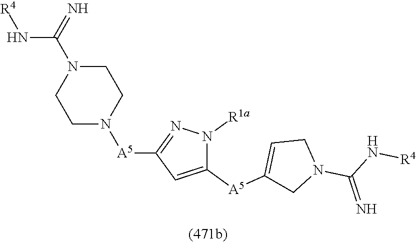

A compound of the formula (463) is reacted with a compound of the formula (464), a known compound or a compound prepared by known methods, in the presence of a palladium catalyst such as palladium (II) acetate, tetrakis(triphenylphosphine)palladium(0), dichlorobis (triphenylphosphine) palladium(II), palladium on carbon, bis(acetonitrile)dichloropalladium(II), and the like, in the presence of an organophosphine such as 2-dicyclohexylphosphino-2′,6′-dimethoxybiphenyl, 2-dicyclohexylphosphino-2′-(N,N-dimethylamino)biphenyl, 2-dicyclohexylphosphino-2′,4′,6′-triisopropylbiphenyl, 2-di-tert-butylphosphino-2′,4′,6′-triisopropylbiphenyl, (2-biphenyl)dicyclohexyl phosphine, (2-biphenyl)di-tert-butylphosphine, 2-dicyclohexylphosphino-2′,6′-diisopropoxybiphenyl, 2-di-tert-butylphosphino-3,4,5,6-tetramethyl-2′,4′,6′-triisopropyl-1,1′-biphenyl. Sodium 2′-dicyclo hexylphosphino-2,6-dimethoxy-1,1′-biphenyl-3-sulfonate, 2-di-tert-butylphosphino-2′-methyl biphenyl, 2-dicyclohexylphosphino-2′-methylbiphenyl, 2′-(di-tert-butyl phosphino)-N,N-dimethyl biphenyl-2-amine, 2′-(diphenylphosphino)-N,N′-dimethyl-(1,1′-biphenyl)-2-amine, and the like, in the presence of a base such as sodium carbonate, lithium carbonate, potassium carbonate, cesium carbonate, sodium hydroxide, lithium hydroxide, potassium hydroxide, triethylamine, N,N-diisopropylethylamine, pyridine, 2,6-dimethylpyridine, and the like, in a solvent such as tetrahydrofuran, 1,4-dioxane, acetonitrile, methylene chloride, chloroform, 1,2-dichloroethane, 1,2-dimethoxyethane, and the like, optionally with heating, optionally with microwave irradiation to provide a compound of the formula (465). A compound of the formula (465) is reacted with a compound of the formula (466) in the presence of a base such as sodium t-butoxide, potassium t-butoxide, lithium t-butoxide, sodium methoxide, potassium methoxide, and the like in the presence of a palladium catalyst such as tris(dibenzylideneacetone)dipalladium, palladium (ii) acetate, tetrakis(triphenylphosphine)palladium(0), dichlorobis (triphenylphosphine) palladium(II), palladium on carbon, bis(acetonitrile)dichloropalladium(II), and the like, optionally in the presence of an amino-phosphorous compound such as 2,8,9-Triisobutyl-2,5,8,9-tetraaza-1-phosphabicyclo[3.3.3]undecane, 2,8,9-Trimethyl-2,5,8,9-tetraaza-1-phosphabicyclo[3.3.3]undecane, 2,8,9-Triisopropyl-2,5,8,9-tetraza-1-phosphabicyclo[3.3.3]undecane and the like, in the presence of a solvent such as toluene, benzene, xylene, tetrahydrofuran, 1,4-dioxane, acetonitrile, N,N-dimethylformamide, dimethylsulfoxide, N,N-dimethylacetamide, and the like, optionally with heating, optionally with microwave irradiation to provide a compound of the formula (467). A compound of the formula (467) is reacted with an acid such as trifluoroacetic acid, hydrochloric acid, sulfuric acid, and the like, in a solvent such as methylene chloride, 1,2-dichloroethane, tetrahydrofuran, 1,4-dioxane, methanol, ethanol, N,N-dimethylformamide, dimethylsulfoxide, N,N-dimethylacetamide, and the like, optionally in the presence of methylphenyl ether, optionally with heating, optionally with microwave irradiation to provide a compound of the formula (468). A compound of the formula (468) is reacted with a compound of the formula (469) in the presence of a base such as triethylamine, N,N-diisopropylethylamine, pyridine, 2,6-dimethylpyridine, and the like, in the presence of a solvent such as N,N-dimethylformamide, dimethylsulfoxide, N,N-dimethylacetamide, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (470). A compound of the formula (470) is reacted with an acid such as trifluoroacetic acid, hydrochloric acid, sulfuric acid, and the like, in a solvent such as methylene chloride, chloroform, 1,2-dichloroethane, tetrahydrofuran, 1,4-dioxane, methanol, ethanol, N,N-dimethylformamide, dimethylsulfoxide, N,N-dimethylacetamide, and the like, optionally in the presence of methylphenyl ether, optionally with heating, optionally with microwave irradiation to provide a compound of the formula (471). A compound of the formula (471) is reacted with a compound of the formula (471a), a known compound or a compound prepared by known methods, in the presence of a base such as sodium carbonate, potassium carbonate, lithium carbonate, triethylamine, pyridine, and the like, in a solvent such as N,N-dimethylformamide, dimethylsulfoxide, N,N-dimethylacetamide, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (471b).

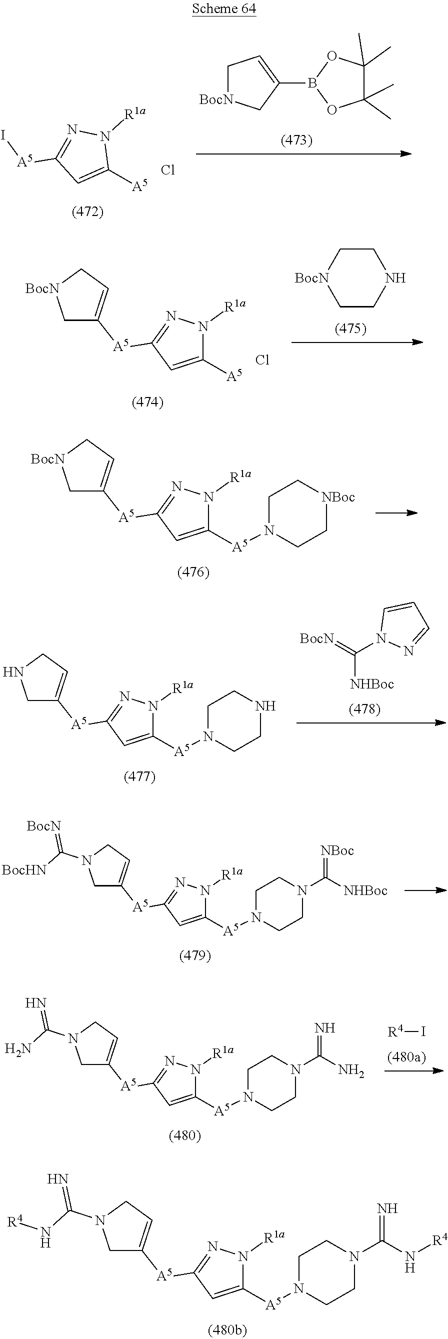

A compound of the formula (472) is reacted with a compound of the formula (473), a known compound or a compound prepared by known methods, in the presence of a palladium catalyst such as palladium (ii) acetate, tetrakis(triphenylphosphine)palladium(0), dichlorobis (triphenylphosphine) palladium(II), palladium on carbon, bis(acetonitrile)dichloropalladium(II), and the like, in the presence of an organophosphine such as 2-dicyclohexylphosphino-2′,6′-dimethoxybiphenyl, 2-dicyclohexylphosphino-2′-(N,N-dimethylamino)biphenyl, 2-dicyclohexylphosphino-2′,4′,6′-triisopropylbiphenyl, 2-di-tert-butylphosphino-2′,4′,6′-triisopropylbiphenyl, (2-biphenyl)dicyclohexyl phosphine, (2-biphenyl)di-tert-butylphosphine, 2-dicyclohexylphosphino-2′,6′-diisopropoxybiphenyl, 2-di-tert-butylphosphino-3,4,5,6-tetramethyl-2′,4′,6′-triisopropyl-1,1′-biphenyl. Sodium 2′-dicyclohexylphosphino-2,6-dimethoxy-1,1′-biphenyl-3-sulfonate, 2-di-tert-butylphosphino-2′-methyl biphenyl, 2-dicyclohexylphosphino-2′-methylbiphenyl, 2′-(di-tert-butylphosphino)-N,N-dimethyl biphenyl-2-amine, 2′-(diphenylphosphino)-N,N′-dimethyl-(1,1′-biphenyl)-2-amine, and the like, in the presence of a base such as sodium carbonate, lithium carbonate, potassium carbonate, cesium carbonate, sodium hydroxide, lithium hydroxide, potassium hydroxide, triethylamine, N,N-diisopropylethylamine, pyridine, 2,6-dimethylpyridine, and the like, in a solvent such as tetrahydrofuran, 1,4-dioxane, acetonitrile, methylene chloride, chloroform, 1,2-dichloroethane, 1,2-dimethoxyethane, and the like, optionally with heating, optionally with microwave irradiation to provide a compound of the formula (474). A compound of the formula (474) is reacted with a compound of the formula (475) in the presence of a base such as sodium t-butoxide, potassium t-butoxide, lithium t-butoxide, sodium methoxide, potassium methoxide, and the like in the presence of a palladium catalyst such as tris(dibenzylideneacetone)dipalladium, palladium (II) acetate, tetrakis(triphenylphosphine)palladium(0), dichlorobis (triphenylphosphine) palladium(II), palladium on carbon, bis(acetonitrile)dichloropalladium(II), and the like, optionally in the presence of an amino-phosphorous compound such as 2,8,9-Triisobutyl-2,5,8,9-tetraaza-1-phosphabicyclo[3.3.3]undecane, 2,8,9-Trimethyl-2,5,8,9-tetraaza-1-phosphabicyclo[3.3.3]undecane, 2,8,9-Triisopropyl-2,5,8,9-tetraza-1-phosphabicyclo[3.3.3]undecane and the like, in the presence of a solvent such as toluene, benzene, xylene, tetrahydrofuran, 1,4-dioxane, acetonitrile, N,N-dimethylformamide, dimethylsulfoxide, N,N-dimethylacetamide, and the like, optionally with heating, optionally with microwave irradiation to provide a compound of the formula (476). A compound of the formula (476) is reacted with an acid such as trifluoroacetic acid, hydrochloric acid, sulfuric acid, and the like, in a solvent such as methylene chloride, 1,2-dichloroethane, tetrahydrofuran, 1,4-dioxane, methanol, ethanol, N,N-dimethylformamide, dimethylsulfoxide, N,N-dimethylacetamide, and the like, optionally in the presence of methylphenyl ether, optionally with heating, optionally with microwave irradiation to provide a compound of the formula (477). A compound of the formula (477) is reacted with a compound of the formula (478) in the presence of a base such as triethylamine, N,N-diisopropylethylamine, pyridine, 2,6-dimethylpyridine, and the like, in the presence of a solvent such as N,N-dimethylformamide, dimethylsulfoxide, N,N-dimethylacetamide, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (479). A compound of the formula (479) is reacted with an acid such as trifluoroacetic acid, hydrochloric acid, sulfuric acid, and the like, in a solvent such as methylene chloride, chloroform, 1,2-dichloroethane, tetrahydrofuran, 1,4-dioxane, methanol, ethanol, N,N-dimethylformamide, dimethylsulfoxide, N,N-dimethylacetamide, and the like, optionally in the presence of methylphenyl ether, optionally with heating, optionally with microwave irradiation to provide a compound of the formula (480). A compound of the formula (480) is reacted with a compound of the formula (480a), a known compound or a compound prepared by known methods, in the presence of a base such as sodium carbonate, potassium carbonate, lithium carbonate, triethylamine, pyridine, and the like, in a solvent such as N,N-dimethylformamide, dimethylsulfoxide, N,N-dimethylacetamide, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (480b).

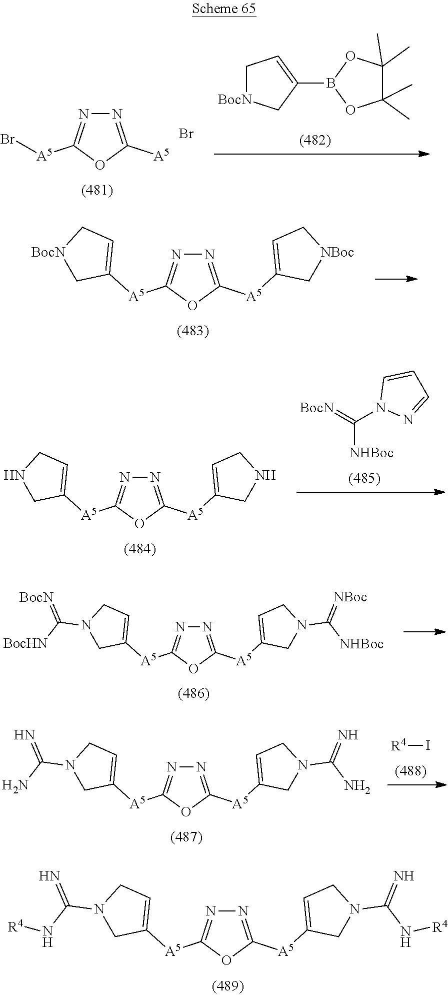

A compound of the formula (481) is reacted with a compound of the formula (482), a known compound or a compound prepared by known methods, in the presence of a palladium catalyst such as palladium (II) acetate, tetrakis(triphenylphosphine)palladium(0), dichlorobis (triphenylphosphine) palladium(II), palladium on carbon, bis(acetonitrile)dichloropalladium(II), and the like, in the presence of an organophosphine such as 2-dicyclohexylphosphino-2′,6′-dimethoxybiphenyl, 2-dicyclohexylphosphino-2′-(N,N-dimethylamino)biphenyl, 2-dicyclohexylphosphino-2′,4′,6′-triisopropylbiphenyl, 2-di-tert-butylphosphino-2′,4′,6′-triisopropylbiphenyl, (2-biphenyl)dicyclohexyl phosphine, (2-biphenyl)di-tert-butylphosphine, 2-dicyclohexylphosphino-2′,6′-diisopropoxybiphenyl, 2-di-tert-butylphosphino-3,4,5,6-tetramethyl-2′,4′,6′-triisopropyl-1,1′-biphenyl, Sodium 2′-dicyclohexylphosphino-2,6-dimethoxy-1,1′-biphenyl-3-sulfonate, 2-di-tert-butylphosphino-2′-methyl biphenyl, 2-dicyclohexylphosphino-2′-methylbiphenyl, 2′-(di-tert-butyl phosphino)-N,N-dimethyl biphenyl-2-amine, 2′-(diphenylphosphino)-N,N′-dimethyl-(1,1′-biphenyl)-2-amine, and the like, in the presence of a base such as sodium carbonate, lithium carbonate, potassium carbonate, cesium carbonate, sodium hydroxide, lithium hydroxide, potassium hydroxide, triethylamine, N,N-diisopropylethylamine, pyridine, 2,6-dimethylpyridine, and the like, in a solvent such as tetrahydrofuran, 1,4-dioxane, acetonitrile, methylene chloride, chloroform, 1,2-dichloroethane, 1,2-dimethoxyethane, and the like, optionally with heating, optionally with microwave irradiation to provide a compound of the formula (483). A compound of the formula (483) is reacted with an acid such as trifluoroacetic acid, hydrochloric acid, sulfuric acid, and the like, in a solvent such as methylene chloride, 1,2-dichloroethane, tetrahydrofuran, 1,4-dioxane, methanol, ethanol, N,N-dimethylformamide, dimethylsulfoxide, N,N-dimethylacetamide, and the like, optionally in the presence of methylphenyl ether, optionally with heating, optionally with microwave irradiation to provide a compound of the formula (484). A compound of the formula (484) is reacted with a compound of the formula (485) in the presence of a base such as triethylamine, N,N-diisopropylethylamine, pyridine, 2,6-dimethylpyridine, and the like, in the presence of a solvent such as N,N-dimethylformamide, dimethylsulfoxide, N,N-dimethylacetamide, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (486). A compound of the formula (486) is reacted with an acid such as trifluoroacetic acid, hydrochloric acid, sulfuric acid, and the like, in a solvent such as methylene chloride, chloroform, 1,2-dichloroethane, tetrahydrofuran, 1,4-dioxane, methanol, ethanol, N,N-dimethylformamide, dimethylsulfoxide, N,N-dimethylacetamide, and the like, optionally in the presence of methylphenyl ether, optionally with heating, optionally with microwave irradiation to provide a compound of the formula (487). A compound of the formula (487) is reacted with a compound of the formula (488), a known compound or a compound prepared by known methods, in the presence of a base such as sodium carbonate, potassium carbonate, lithium carbonate, triethylamine, pyridine, and the like, in a solvent such as N,N-dimethylformamide, dimethylsulfoxide, N,N-dimethylacetamide, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (489).

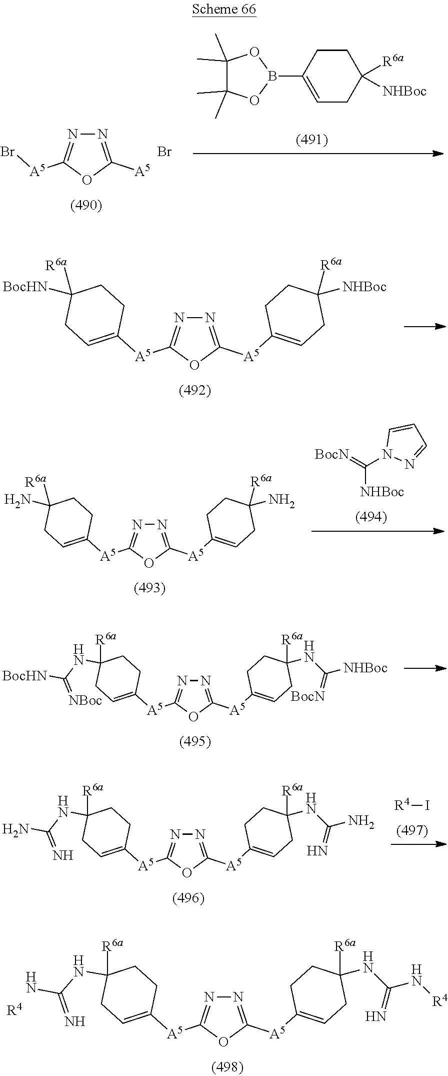

A compound of the formula (490) is reacted with a compound of the formula (491), a known compound or a compound prepared by known methods, in the presence of a palladium catalyst such as palladium (ii) acetate, tetrakis(triphenylphosphine)palladium(0), dichlorobis (triphenylphosphine) palladium(II), palladium on carbon, bis(acetonitrile)dichloropalladium(II), and the like, in the presence of an organophosphine such as 2-dicyclohexylphosphino-2′,6′-dimethoxybiphenyl, 2-dicyclohexylphosphino-2′-(N,N-dimethylamino)biphenyl, 2-dicyclohexylphosphino-2′,4′,6′-triisopropylbiphenyl, 2-di-tert-butylphosphino-2′,4′,6′-triisopropylbiphenyl, (2-biphenyl)dicyclohexyl phosphine, (2-biphenyl)di-tert-butylphosphine, 2-dicyclohexylphosphino-2′,6′-diisopropoxybiphenyl, 2-di-tert-butylphosphino-3,4,5,6-tetramethyl-2′,4′,6′-triisopropyl-1,1′-biphenyl. Sodium 2′-dicyclohexylphosphino-2,6-dimethoxy-1,1′-biphenyl-3-sulfonate, 2-di-tert-butylphosphino-T-methyl biphenyl, 2-dicyclohexylphosphino-2′-methylbiphenyl, 2′-(di-tert-butylphosphino)-N,N-dimethyl biphenyl-2-amine, 2′-(diphenylphosphino)-N,N′-dimethyl-(1,1′-biphenyl)-2-amine, and the like, in the presence of a base such as sodium carbonate, lithium carbonate, potassium carbonate, cesium carbonate, sodium hydroxide, lithium hydroxide, potassium hydroxide, triethylamine, N,N-diisopropylethylamine, pyridine, 2,6-dimethylpyridine, and the like, in a solvent such as tetrahydrofuran, 1,4-dioxane, acetonitrile, methylene chloride, chloroform, 1,2-dichloroethane, 1,2-dimethoxyethane, and the like, optionally with heating, optionally with microwave irradiation to provide a compound of the formula (492). A compound of the formula (492) is reacted with an acid such as trifluoroacetic acid, hydrochloric acid, sulfuric acid, and the like, in a solvent such as methylene chloride, 1,2-dichloroethane, tetrahydrofuran, 1,4-dioxane, methanol, ethanol, N,N-dimethylformamide, dimethylsulfoxide, N,N-dimethylacetamide, and the like, optionally in the presence of methylphenyl ether, optionally with heating, optionally with microwave irradiation to provide a compound of the formula (493). A compound of the formula (493) is reacted with a compound of the formula (494) in the presence of a base such as triethylamine, N,N-diisopropylethylamine, pyridine, 2,6-dimethylpyridine, and the like, in the presence of a solvent such as N,N-dimethylformamide, dimethylsulfoxide, N,N-dimethylacetamide, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (495). A compound of the formula (495) is reacted with an acid such as trifluoroacetic acid, hydrochloric acid, sulfuric acid, and the like, in a solvent such as methylene chloride, chloroform, 1,2-dichloroethane, tetrahydrofuran, 1,4-dioxane, methanol, ethanol, N,N-dimethylformamide, dimethylsulfoxide, N,N-dimethylacetamide, and the like, optionally in the presence of methylphenyl ether, optionally with heating, optionally with microwave irradiation to provide a compound of the formula (496). A compound of the formula (496) is reacted with a compound of the formula (497), a known compound or a compound prepared by known methods, in the presence of a base such as sodium carbonate, potassium carbonate, lithium carbonate, triethylamine, pyridine, and the like, in a solvent such as N,N-dimethylformamide, dimethylsulfoxide, N,N-dimethylacetamide, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (498).

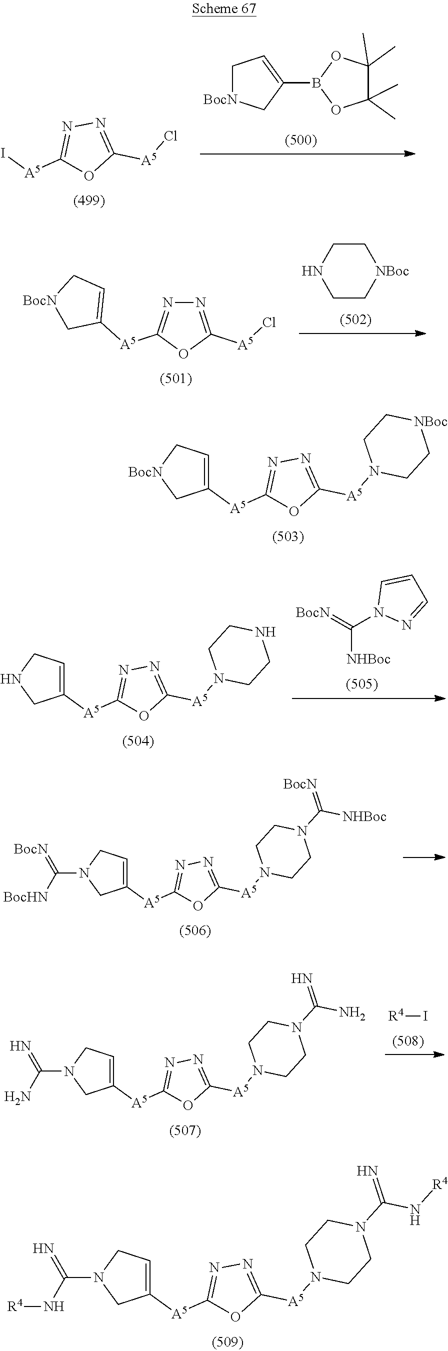

A compound of the formula (499) is reacted with a compound of the formula (500), a known compound or a compound prepared by known methods, in the presence of a palladium catalyst such as palladium (II) acetate-tetrakis(triphenylphosphine)palladium(0), dichlorobis (triphenylphosphine) palladium(II), palladium on carbon, his(acetonitrile)dichloropalladium(II), and the like, in the presence of an organophosphine such as 2-dicyclohexylphosphino-2′,6′-dimethoxybiphenyl, 2-dicyclohexylphosphino-2′-(N,N-dimethylamino)biphenyl, 2-dicyclohexylphosphino-2′,4′,6′-triisopropylbiphenyl, 2-di-tert-butylphosphino-2′,4′,6′-triisopropylbiphenyl, (2-biphenyl)dicyclohexyl phosphine, (2-biphenyl)di-tert-butylphosphine, 2-dicyclohexylphosphino-2′,6′-diisopropoxybiphenyl, 2-di-tert-butylphosphino-3,4,5,6-tetramethyl-2′,4′,6′-triisopropyl-1,1′-biphenyl, Sodium 2′-dicyclo hexylphosphino-2,6-dimethoxy-1,1′-biphenyl-3-sulfonate, 2-di-tert-butylphosphino-2′-methyl biphenyl, 2-dicyclohexylphosphino-2-methylbiphenyl, 2′-(di-tert-butylphosphino)-N,N-dimethyl biphenyl-2-amine, 2′-(diphenylphosphino)-N,N′-dimethyl-(1,1′-biphenyl)-2-amine, and the like, in the presence of a base such as sodium carbonate, lithium carbonate, potassium carbonate, cesium carbonate, sodium hydroxide, lithium hydroxide, potassium hydroxide, triethylamine, N,N-diisopropylethylamine, pyridine, 2,6-dimethylpyridine, and the like, in a solvent such as tetrahydrofuran, 1,4-dioxane, acetonitrile, methylene chloride, chloroform, 1,2-dichloroethane, 1,2-dimethoxyethane, and the like, optionally with heating, optionally with microwave irradiation to provide a compound of the formula (501). A compound of the formula (501) is reacted with a compound of the formula (502) in the presence of a base such as sodium t-butoxide, potassium t-butoxide, lithium t-butoxide, sodium methoxide, potassium methoxide, and the like in the presence of a palladium catalyst such as tris(dibenzylideneacetone)dipalladium, palladium (II) acetate, tetrakis(triphenylphosphine)palladium(0), dichlorobis (triphenylphosphine) palladium(II), palladium on carbon, bis(acetonitrile)dichloropalladium(II), and the like, optionally in the presence of an amino-phosphorous compound such as 2,8,9-Triisobutyl-2,5,8,9-tetraaza-1-phosphabicyclo[3.3.3]undecane, 2,8,9-Trimethyl-2,5,8,9-tetraaza-1-phosphabicyclo[3.3.3]undecane, 2,8,9-Triisopropyl-2,5,8,9-tetraza-1-phosphabicyclo[3.3.3]undecane and the like, in the presence of a solvent such as toluene, benzene, xylene, tetrahydrofuran, 1,4-dioxane, acetonitrile, N,N-dimethylformamide, dimethylsulfoxide, N,N-dimethylacetamide, and the like, optionally with heating, optionally with microwave irradiation to provide a compound of the formula (503). A compound of the formula (503) is reacted with an acid such as trifluoroacetic acid, hydrochloric acid, sulfuric acid, and the like, in a solvent such as methylene chloride, 1,2-dichloroethane, tetrahydrofuran, 1,4-dioxane, methanol, ethanol, N,N-dimethylformamide, dimethylsulfoxide, N,N-dimethylacetamide, and the like, optionally in the presence of methylphenyl ether, optionally with heating, optionally with microwave irradiation to provide a compound of the formula (504). A compound of the formula (504) is reacted with a compound of the formula (505) in the presence of a base such as triethylamine, N,N-diisopropylethylamine, pyridine, 2,6-dimethylpyridine, and the like, in the presence of a solvent such as N,N-dimethylformamide, dimethylsulfoxide, N,N-dimethylacetamide, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (506). A compound of the formula (506) is reacted with an acid such as trifluoroacetic acid, hydrochloric acid, sulfuric acid, and the like, in a solvent such as methylene chloride, chloroform, 1,2-dichloroethane, tetrahydrofuran, 1,4-dioxane, methanol, ethanol, N,N-dimethylformamide, dimethylsulfoxide, N,N-dimethylacetamide, and the like, optionally in the presence of methylphenyl ether, optionally with heating, optionally with microwave irradiation to provide a compound of the formula (507). A compound of the formula (507) is reacted with a compound of the formula (508), a known compound or a compound prepared by known methods, in the presence of a base such as sodium carbonate, potassium carbonate, lithium carbonate, triethylamine, pyridine, and the like, in a solvent such as N,N-dimethylformamide, dimethylsulfoxide, N,N-dimethylacetamide, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (509).

The Examples provided below provide representative methods for preparing exemplary compounds of the present invention. The skilled practitioner will know how to substitute the appropriate reagents, starting materials and purification methods known to those skilled in the art, in order to prepare the compounds of the present invention.

The following LC/MS procedure was used for the analysis of the examples described herein. LC/MS data were determined with a Waters Alliance 2695 HPLC/MS (Waters Symmetry C18, 4.6×75 mm, 3.5 μm) with a 2996 diode array detector from 210-400 nm; the solvent system is 5-95% acetonitrile in water (with 0.1% trifluoroacetic acid) over nine minutes using a linear gradient, and retention times are in minutes. Mass spectrometry was performed on a Waters ZQ using electrospray in positive mode.

Preparative reverse phase HPLC was performed on a Phenomenex LUNA column (19×100 mm, C18, 5 μm) with a 10 minute mobile phase gradient of 10% acetonitrile/water to 90% acetonitrile/water with 0.1% trifluoroacetic acid as buffer using 214 and 254 nm as detection wavelengths. Injection and fraction collection were performed with a Gilson 215 liquid handling apparatus using Trilution LC software.

¹H-NMR's were taken on a Varian 300 MHz NMR using tetramethylsilane (TMS) as internal standard (δ=0.00) with peaks reported downfield from TMS.

The examples provide methods for preparing representative compounds of formulas (I) through (XXXXVII). The skilled practitioner will know how to substitute the appropriate reagents, starting materials and purification methods known to those skilled in the art, in order to prepare additional compounds of the present invention.

General experimental procedures: The general experimental procedures described herein can be used by one skilled in the art to prepare the compounds of the disclosure and the intermediates necessary for preparation of the compounds of the disclosure.

### General Experimental 1: Synthesis of 1,2,3-Triazoles

Step 1: Synthesis of arylsulfonylarylhydrazone: A glass container was charged with 1.0 equivalents of an aryllsulfonylhydrazide, 1.0 equivalents of an acetophenone derivative and sufficient methanol to make a 0.1 molar solution and stirred for 18 hours at 23° C. The solid product is collected by filtration, washed with methanol, dried, and used without further purification.

Step 2: 1,2,3-triazole ring formation: A glass container was charged with 1.0 equivalents of an arylsulfonylarylhydrazone, 1.0 equivalents of an aniline derivative, 1.0 equivalents pivalic acid and 0.5 equivalents copper acetate in sufficient toluene to make a 0.2 molar solution. The slurry was heated to 95° C. for 15 hours. The reaction was cooled and methylene chloride was added and the resulting solution was loaded onto a silica gel column and purified using a gradient of methylene chloride/hexane (50 methylene chloride to methylene chloride 100%).

### General Synthesis 2: Synthesis of 1-Alkyl-1,2,4-Triazole

A glass container was charged with 1.0 equivalents an arylcarboxylic acid, sufficient dry N,N-dimethylformamide to make a 0.6 molar solution and 3.00 equivalents N, N-diisopropylethyl amine. After five minutes of stirring 1.2 equivalents of (1-[bis(dimethylamino)methylene]-1H-1,2,3-triazolo[4,5-b]pyridinium 3-oxide hexafluorophosphate (HATU) added and the reaction was stirred for 40 minutes. Then an aryl amidine HCl (1.0 equivalents) was added and reaction stirred at 23° C. for 18 hours. The reaction was then diluted with 30 mL ethyl acetate and washed with three 20 mL portions of water, dried and concentrated under vacuum. The residual material was dissolved with sufficient glacial acetic acid to make a 1.0 molar solution and 1.3 equivalents of an allylhydrazine added. The reaction was heated at 80° C. for 18 hours. The reaction was then cooled and diluted with two volumes of water per volume of acetic acid. The solid precipitate was collected, rinsed with water, dried and used without further purification.

### General Experimental 3: Synthesis of 4-Alkyl-1,2,4-Triazole

Acid Chloride Synthesis: A glass container was charged with 1.0 equivalents of an arylcarboxylic acid and sufficient chloroform to make a 0.5 molar solution. Then 0.1 equivalents N,N-dimethylformamide was added followed by slow addition of 1.5 equivalents of oxalyl chloride. After stirring for from 3 to 24 hours, the reaction was concentrated under vacuum and used without further purification.

Bis-arylcarbonylhydrazide synthesis: A glass container was charged with 0.5 equivalents of hydrazine mono hydrate, 1.3 equivalents of N, N-diisopropylethyl amine and sufficient chloroform to make a 0.5 molar solution was cooled in an ice water bath. A solution of 1.0 equivalents of an arylcarboxylic acid chloride in sufficient chloroform to make a 1.0 molar solution was added over 15 minutes to 1 hour and stirred for 18 hours at 23° C. The chloroform was removed under vacuum and the residue was stirred with 150 mL of water for 18 hours after brief sonication. The solid material was isolated by decantation/filtration, rinsed with acetonitrile, and dried. The material was used without further purification.

Bis-imidoyl chloride synthesis: A glass container was charged with 1.0 equivalents of a bis(arylcarbonyl) hydrazide and slurried with sufficient toluene to make a 0.2 molar solution and brief sonication. Then 3.0 equivalents of PCl₅ was added in three portions. The reaction was heated to 100° C. for 18 hours. The reaction was cooled and concentrated under vacuum to yield a solid and the residual PCl₅ was quenched by addition of wet ice and water (about 8 grams of ice per gram PCl₅). After two hours, the water was removed by decantation, 100 mL of acetonitrile was added, and then removed under vacuum. The residual material was used without further purification.

4-Alkyl, 1,2,4-triazole formation synthesis: A bisimidoyl chloride (1.0 equiv) was slurried with 7 mL methanol per gram of bisimidoyl chloride and 3.3 equivalents of an amine was added either as a neat material or as a solution in ethanol or methanol over ten minutes. The reaction was slowly warmed to 40° C. for 30 minutes and then heated at 65° C. for 18 hours. The reaction mixture was cooled to room temperature and half of the solvent was removed. To the reaction mixture was added water and the mixture was sonicated for 5 minutes and allowed to stir at room temperature for 1 hour. The reaction was filtered and solid was rinsed with water and air dried for 1 hour to isolate the solid. The product was dried at 50° C. for 18 hours and used for the next reaction.

Unsymmetrical biscarbonylaryl hydrazide formation synthesis: A glass container was charged with, 1.0 equivalents of monobenzoylyhydrazide and dissolved with sufficient N,N-dimethylformamide to yield a 0.2 molar solution and 1.16 equivalents of N, N-diisopropylethyl amine added. The solution was cooled in an ice water bath and the reaction was treated with 1.0 equivalents of an acid chloride. The reaction was allowed to stir for 18 hours at 23° C. The reaction was then diluted with 1.5 volumes of water per volume of N,N-dimethylformamide, stirred for 30 minutes, filtered, and the collected solid was washed with water and ether, and dried under high vacuum. The residual material is used without further purification.

### General Experimental 4: Boc Protection of Alcohol

A glass container was charged with 1.00 equivalents of an alcohol and sufficient 1,4-dioxane was added to make a 0.3 molar solution. Then 3 mole percent 4-dimethylaminopyridine was added, followed by 1.2 equivalents of di-tert-butyl dicarbonate and the reaction was warmed at 55° C. for 18 hours. The reaction then was cooled to 23° C., 10 mL of methanol per gram of the alcohol was added, and reaction concentrated under vacuum. The residual material was used without further purification.

General Experimental 5: Beta-Diketone Synthesis:

A glass container was charged with 1.0 equivalents of a bromoaryl alkyl ketone and sufficient dry tetrahydrofuran to yield 0.2 to 0.5 molar solutions. Then 1.25 equivalents of sodium hydride was added in portions and the reaction warmed to 45° C. and an alkyl bromobenzoate added dropwise over two minutes and reaction temp was heated to 60° C. The reaction was allowed to cool after LCMS indicated reaction complete. The reaction was quenched by addition of 2 mL water added over two minutes. Then 1.5 equivalents of 2.0 N HCl was added over five minutes and solids formed. After one hour the solids were collected, air dried and used without further purification.

General Experimental 6: Buchwald Hartwig Coupling:

A glass container was charged with 1.0 equivalents of an arylbromide, 4.0 equivalents of mono-Boc-protected diamine, 0.04 equivalents per arylbromide of Palladium acetate, 0.10 equivalents per arylbromide of 2-dicyclohexylphosphino-2′,6′-dimethoxybiphenyl, and sufficient 1,4-dioxane to form a solution of 0.1 to 0.3 molar concentrations in 1,4-dioxane. Then 3.0 equivalents of cesium carbonate per arylbromide was added, and the reaction deoxygenated for several minutes. The reaction was heated to 95° C. for 3 to 18 hours. When the reaction is complete as indicated by LC/MS, the reaction was allowed to cool to 23° C., and diluted with two volumes of ethyl acetate per volume of 1,4-dioxane and three volumes of water. After 30 minutes, the solid precipitate is collected by filtration, and ethyl acetate phase dried over MgSO₄, filtered, and concentrated under vacuum. Both the solid precipitate and the concentrated ethyl acetate extracts were combined and deprotected using the procedure described in general experimental 8.

### General Experimental 7: Guanylation

A glass container was charged with an amine salt and 4 mL of N,N-dimethylformamide per gram. Then 3 to 4 equivalents of triethylamine per equivalent of the amine is added over 2 to 10 minutes. After 5 minutes of stirring, 1.1 to 1.6 equivalents per amine of tert-butyl (1H-pyrazol-1-yl)methanediylidenedicarbamate was added as a solid and the reaction is stirred for 18 hours at up to 45° C. When LCMS indicated reaction is complete, the triethylamine was removed under vacuum. The reaction was diluted with ethyl acetate, washed with water three times, dried with MgSO₄, filtered, and the solution was concentrated under vacuum. The residual material is purified by normal phase chromatography using methylene chloride ethyl acetate. Alternatively, the residual material is purified by normal phase chromatography using methylene chloride/methanol.

In some embodiments, N,N-diisopropylethyl amine is used in place of the triethylamine.

In some embodiments, methanol can be used in place of methylene chloride

In some embodiments, methanol can be used in place of N,N-dimethylformamide

In some embodiments, tetrahydrofuran can be used in place of methylene chloride.

In some embodiments, N,N-dimethylformamide is added in combination with N, N-diisopropylethyl amine.

In some embodiments the residual material is purified by chromatography with hexanes/ethyl acetate.

### General Experimental 8: Deprotection of Bis(Boc Protected Amine)

A glass container was charged with 1.0 equivalents of a bis(boc protected amine) and slurried with 2 to 5 mL of methylene chloride per gram. Then 2 to 5 equivalents of anisole is added and 16 to 50 equivalents of trifluoroacetic acid per equivalent of bis(boc protected amine) is added. The reaction was stirred until complete by LCMS. Then the reaction was concentrated under vacuum and triturated with 5 to 20 mL of ether per gram of product, dried under vacuum, and then used without further purification.

In some embodiments, the anisole can be omitted.

In some embodiments, the anisole is omitted and the trifluoroacetic acid is replaced with 4.0 N HCl in 1,4-dioxane.

In some embodiments, when the reaction is complete by LC/MS the reaction is diluted with four volumes of ether per volume of 1,4-dioxane, the reaction is stirred briefly, and the solvents are decanted. The remaining solid is dried under vacuum.

### General Experimental 9: Deprotection of Bis(Diboc Protected Guanidine)

A glass container was charged with 1.0 equivalents of a bis(diboc protected guanidine) and slurried with 2 to 5 mL methylene chloride per gram. Then 2 to 5 equivalents of anisole is added and 20 to 50 equivalents of trifluoroacetic acid added per equivalents of protected bis(diboc protected guanidine) is added. The reaction was stirred until complete by LCMS and then concentrated under vacuum. The residual material is triturated with 5 to 20 mL of ether per grain of product, dried under vacuum, and the purified by semi-prep reverse phase HPLC.

In some embodiments, the anisole is omitted.

In some embodiments, the anisole is omitted and 4.0 N HCl in 1,4-dioxane is used in place of the trifluoroacetic acid.

In some embodiments, when the reaction is complete by LC/MS the reaction is diluted with four volumes of ether per volume of 1,4-dioxane, the reaction is stirred briefly, and the solvents are decanted. The remaining solid is dried under vacuum.

### General Experimental 10: Synthesis of 1,3,4-Isooxazole

Symmetrical biscarbonylaryl hydrazide formation: A glass container was charged with 1.0 equivalents of an arylcarboxylic acid and sufficient chloroform to make a 0.5 molar solution. Then 0.1 equivalents N,N-dimethylformamide was added followed by slow addition of 1.5 equivalents of oxalyl chloride. After stirring for from 3 to 24 hours, the reaction was concentrated under vacuum and the resulting arylcarboxylic acid chloride was used without further purification.

Bis-arylcarbonylhydrazide synthesis: A glass container was charged with 0.5 equivalents of hydrazine mono hydrate, 1.3 equivalents of N, N-diisopropylethyl amine and sufficient chloroform to make a 0.5 molar solution, then was cooled in an ice water bath. A solution of 1.0 equivalents of an arylcarboxylic acid chloride in sufficient chloroform to make a 1.0 molar solution was added over 15 minutes to 1 hour and the reaction was stirred for 18 hours at 23° C. The chloroform was removed under vacuum, 150 mL of water was added, the mixture was briefly sonicated, and the reaction was stirred at 23° C. for 18 hours. The solid precipitate was collected by filtration, washed with acetonitrile, and dried under vacuum. The resulting bis-arylcarbonylhydrazide was used without further purification.

1,3,4-Oxadiazole synthesis method 1: A glass container was charged with a bis-arylcarbonylhydrazide and phosphonisoxychloride (10 mL per gram) was added. The suspension was heated to reflux for 20 hours. Then reaction was cooled to 0° C., and solid ice is added to the reaction (6 grams of ice per mL of phosphorusoxychloride). After 30 minutes the reaction is neutralized with solid sodium carbonate, filtered and the resulting material is washed with water. The residual material is suspended in dimethyl sulfoxide, the suspension is filtered, and the remaining material is washed with dimethyl sulfoxide and water, and dried at 60° C. under high vacuum. The resulting 1,3,4-oxadiazole is used without further purification.

1,3,4-Oxadiazole synthesis method 2: A glass container was charged with a biscarbonylaryl hydrazide hydrate and 3.0 equivalents of phosphoruspentachloride in sufficient toluene to make a 0.5 molar solution. The reaction was then heated to 95° C. for 18 hours. The reaction was then cooled, concentrated under vacuum, quenched with ice (10 grams of ice per gram of phosphoruspentachloride), and stirred for 30 minutes. The resulting suspension was filtered, and the collected solid was washed with water and ether. The product was suspended in 10 mL of acetonitrile per gram of product and then the acetonitrile was removed under vacuum.

Unsymmetrical biscarbonylaryl hydrazide synthesis: A glass container was charged with 1.0 equivalents of monobenzoylydrazide and dissolved with sufficient N,N-dimethylformamide to yield a 0.2 molar solution and 0.1.16 equivalents of N, N-diisopropylethyl amine added. The solution was cooled in an ice water bath and 1.0 equivalents of an acid chloride was added. The reaction was allowed to stir for 18 hours at 23° C. The reaction was then diluted with 1.5 volumes of water per volume of N,N-dimethylformamide, stirred for 30 minutes, and filtered. The solid was washed with water and ether, dried under vacuum and the residual material is used without further purification.

### General Experimental 11: Pyrazole Synthesis

A glass container was charged with 1.0 equivalents of a beta diketone in sufficient ethanol to yield a 1.0 molar solution. Then 0.5 equivalents of acetic acid is added followed by addition of a substituted hydrazine (1.0 equivalents) and 1.0 equivalents of N, N-diisopropylethyl amine is added to the reaction. The reaction is heated to 80° C. and stirred for 18 hours, then cooled to 23° C., and concentrated under vacuum. The residual material is partitioned between ethyl acetate and water, the layers are separated, and the organic phase is dried over MgSO₄, filtered, and concentrated under vacuum. The residual material was used without further purification.

### General Experimental 12: Hydrogenation of Double Bond

A glass container was charged with 1.0 equivalents of bis(vinylpiperidine)amine-bis-trifluoroacetic acid salt and dissolved in 20 mL of methanol per gram of diamine salt. Then the reaction was deoxygenated with a nitrogen sweep. Then 10% Pt on carbon (30% weight of diamine salt) was added, the reaction placed under a hydrogen atmosphere balloon and stirred for 3 to 16 hours. The reaction was then filtered to remove the Pt catalyst, and the resulting solution was concentrated under vacuum to yield the desired product.

In some embodiments, the 10% Pt on carbon is replaced with 10% Pd on carbon.

In some embodiments, the reaction is conducted at 45 PSI of hydrogen instead of 1 atmosphere of hydrogen.

### General Experimental 13: 1,2,3-Triazole 3+2 Huigen Reaction

A glass container was charged with an aryltrimethylsilylacetylene (1.02 equiv), 0.1 equivalents of copper sulfate, and 0.20 equivalents of sodium ascorbate. Sufficient tetrahydrofuran was added to make a 0.25 molar solution and equal volumes (compared to the tetrahydrofuran) of methanol and water are added, followed by KF (3.8 equiv). An aryl azide solution (0.5 M in tetrahydrofuran) was added over five minutes and the reaction stirred for 18 hours at 23° C. The reaction was then warmed to 35° C., stirred for an additional 18 hours, and cooled. The reaction was then filtered, and the solid material was used without further purification.

### General Experimental 14: Suzuki Reaction

A glass container was charged with an aryl halide (1.0 equivalents), palladium acetate (0.04 equivalents per aryl halide), 2-dicyclohexylphosphino-2′,6′-dimethoxybiphenyl (0.10 equivalents per aryl), a pinacol boronate (1.20 equivalents per halide) and mixed with sufficient 1,4-dioxane to make 0.2 molar concentration. Then a 2.0 M solution of potassium carbonate (3.75 equivalents per reactive halide) was added and the reaction deoxygenated with nitrogen stream for 5 minutes and heated to 95° C. for 18 hours. The reaction was allowed to cool to 23° C. and is diluted with 2 mL ethyl acetate per mL 1,4-dioxane and 2 mL of water per 1 mL of potassium carbonate solution, stirred for one hour, filtered, and the solid material is purified by silica gel chromatography with an ethyl acetate/methylene chloride gradient.

In some embodiments, after the reaction has stirred at 95° C. for 18 hours, the reaction is cooled to 23° C. and diluted with 2 mL ethyl acetate per mL of 1,4-dioxane and 2 mL of water per 1 mL of potassium carbonate solution. The layers are separated, and the organic phase was dried with sodium sulfate, filtered, and concentrated under vacuum. The residual material is purified by silica gel chromatography with an ethyl acetate/methylene chloride gradient.

In some embodiments, the silica gel chromatography is omitted, and the residual material is used without further purification.

In some embodiments, sodium carbonate is used in place of potassium carbonate and bis(triphenylphosphine)palladium chloride is used in place of palladium acetate, and the 2-dicyclohexylphosphino-2′,6′-dimethoxybiphenyl.

In some embodiments, the sufficient 1,4-dioxane to make 0.2 molar concentration was replaced with sufficient 1,4-dioxane to make 0.15 molar.

In some embodiments, after the reaction is cooled to 23° C., it is diluted with 2 mL dichloromethane per mL of 1,4-dioxane and 2 mL of water per 1 mL of potassium carbonate solution. The layers are separated, and the aqueous layer is a extract with an equal amount of dichloromethane. The organic layers are combined, dried with sodium sulfate, filtered, and concentrated under vacuum. The residual material is triturated with ethyl acetate and used without further purification.

The following compounds were prepared using the methods and techniques described herein. One skilled in the art would know and understand how to select appropriate reagents to re are the compounds described herein.

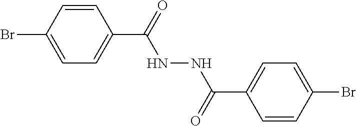 [SMILES: O=C(NNC(=O)c1ccc(Br)cc1)c1ccc(Br)cc1]

Synthesis of 4-Bromo-N′-(4-bromobenzoyl)benzohydrazide: A flask was charged with 50 mL of chloroform and cooled in an ice water bath and 1.14 g (22.3 mmol) of hydrazine monohydrate (7.65 g, 59.2 mmol) of N,N-diisopropylethylamine was added. A solution of 10 g (45.5 mmol) of 4-bromobenzoyl chloride in 50 mL of chloroform was added over one hour and stirred for 18 hours at 23° C. Removed chloroform under vacuum and stir with 150 mL of water for 18 hours after brief sonication. Solids isolated by decantation and filtration. Filter cake/flask was rinsed with 30 mL acetonitrile. Solids from filter added to solids from flask and two 120 mL portions of acetonitrile added and removed under vacuum to dry solid. Yield was 7.1 grams (78%).

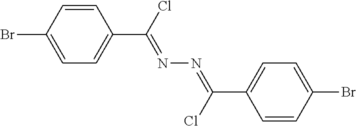 [SMILES: Cl/C(=N\N=C(/Cl)c1ccc(Br)cc1)c1ccc(Br)cc1]

Synthesis of (Z)-4-Bromo-N—((Z)-(4-bromophenyl)chloromethylene)benzo hydrazonoyl chloride (Intermediate A): A 250 mL flask was charged with 15.4 g (38.1 mmol) of 4-bromo-N′-(4-bromobenzoyl)benzohydrazide. The solid was slurried with 100 toluene and briefly sonicated. 24.3 g (116.6 mmol) of PCl₅ was added in three roughly equal portions. The reaction was heated to 100° C. for 18 hours. The reaction was cooled and concentrated under vacuum to yield a solid. The reaction was quenched by addition of wet ice and water (about 50 grams). After a two-hour age water removed by decantation and two 100 mL portions of acetonitrile added and removed under vacuum to remove water. Yield 14.1 grams of solid (83%).

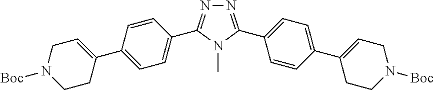 [SMILES: Cn1c(-c2ccc(C3=CCN(C(=O)OC(C)(C)C)CC3)cc2)nnc1-c1ccc(C2=CCN(C(=O)OC(C)(C)C)CC2)cc1]

Synthesis of tert-Butyl 4,4′-(4,4′-(4-methyl-4H-1,2,4-triazole-3,5-diyl)bis(4,1-phenylene))bis(5,6-dihydropyridine-1(2H)-carboxylate): A mixture of 3,5-bis(4-bromo phenyl)-4-methyl-4H-1,2,4-triazole (50 mg, 0.13 mmol), tert-butyl 4-(4,4,5,5-tetramethyl-1,3,2-dioxaborolan-2-yl)-1,2,3,6-tetrahydropyridine-1-carboxylate (94 mg, 0.30 mmol), Dicyclohexyl(2′,6′-dimethoxybiphenyl-2-yl) phosphine (8 mg, 20 μmot), Palladium acetate (2.0 mg, 8 μmot), potassium carbonate (108 mg, 0.78 mmol), water (0.4 mL) and 1,4-dioxane (1 mL) was purged with nitrogen, stirred and heated to 90° C. for 20 h. The mixture was diluted with dichloromethane (20 mL) and water (20 mL) and the aqueous layer was extracted with dichloromethane (20 mL). The combined dichloromethane layers were dried (Na₂SO₄) and evaporated. The crude product mixture was chromatographed on silica gel with a gradient of 50% dichloromethane and 50% ethyl acetate to 100% ethyl acetate to leave a beige solid (53 mg, 68%). 1H-NMR (CDCl3) δ 7.73 (d, 4H, J=8.5 Hz), 7.54 (d, 4H, J=8.5 Hz), 6.17 (bra, 2H), 4.12 (m, 4H), 3.74 (s, 3H), 3.68 (m, 4H), 2.58 (m, 4H), 1.51 (s, 18H). LCMS method A Rt=5.70 mins, purity >95%, (M+H)+=599.

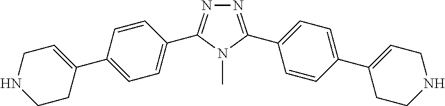 [SMILES: Cn1c(-c2ccc(C3=CCNCC3)cc2)nnc1-c1ccc(C2=CCNCC2)cc1]

Synthesis of 4,4′-(4,4′-(4-Methyl-4H-1,2,4-triazole-3,5-diyl)bis(4,1-phenylene))bis(1,2,3,6-tetrahydropyridine: A solution of tert-butyl 4-{4-[5-(4-{1-[(tert-butoxy)carbonyl]-1,2,3,6-tetrahydropyridin-4-yl}phenyl)-4-methyl-4H-1,2,4-triazol-3-yl]phenyl}-1,2,3,6-tetrahydropyridine-1-carboxylate (28 mg, 48 mop in trifluoroacetic acid (1.5 mL) and dichloromethane (1.5 mL) was stirred for 5 h then evaporated to leave the product as a crystalline solid (46 mg, 100%).

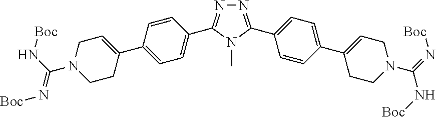 [SMILES: Cn1c(-c2ccc(C3=CCN(/C(=N\C(=O)OC(C)(C)C)NC(=O)OC(C)(C)C)CC3)cc2)nnc1-c1ccc(C2=CCN(/C(=N/C(=O)OC(C)(C)C)NC(=O)OC(C)(C)C)CC2)cc1]

tert-Butyl (4,4′-(4,4′-(4-methyl-4H-1,2,4-triazole-3,5-diyl)bis(4,1-phenylene)) bis(5,6-dihydropyridine-4,1(2H)-diyl))bis(methane-1-yl-1,1-diylidene)tetracarbamate prepared from 4,4′-(4,4′-(4-methyl-4H-1,2,4-triazole-3,5-diyl)bis(4,1-phenylene))bis(1,2,3,6-tetrahydropyridine.

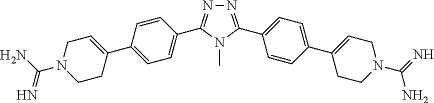 [SMILES: Cn1c(-c2ccc(C3=CCN(C(=N)N)CC3)cc2)nnc1-c1ccc(C2=CCN(C(=N)N)CC2)cc1]

The invention will be illustrated in more detail with reference to the following Examples, but it should be understood that the present invention is not deemed to be limited thereto, it will be apparent to one skilled in the art that various changes and modifications can be made therein without departing from the spirit and scope thereof.

### Example 1

Synthesis of 4,4-(4,4-(4-Methyl-4H-1,2,4-triazole-3,5-diyl)bis(4,1-phenylene))bis(5,6-dihydropyridine-1(2H)-carboximidamide): A solution of 4-(4-{4-methyl-5-[4-(1,2,3,6-tetrahydropyridin-4-yl)phenyl]-4H-1,2,4-triazol-3-yl-}phenyl)-1,2,3,6-tetrahydropyridine (81 mg, 0.11 mmol), N—((N′,N″-bis-tert-butyloxycarbonyl) amidino)pyrrazole (102 mg, 0.33 mmol) and N,N-(diisopropyl)ethylamine (115 mg, 0.88 mmol, 158 μl) in methanol (1.7 mL) was stirred for 4 days and evaporated to dryness. The crude product was dissolved in trifluoroacetic acid (2 mL) and dichloromethane (2 mL) and stirred for 4 hours and the solvents were evaporated. The crude product was purified by preparative reverse phase HPLC and the product fractions were combined and lyophilized to leave a white powder. (17 mg, 19%). ¹H NMR (300 MHz, DMSO-d₆) δ=7.78-7.64 (m, 7H), 7.46 (s, 7H), 6.35 (s, 2H), 4.10 (br d, J=3.3 Hz, 3H), 3.75-3.56 (m, 4H), 3.49-3.40 (m, 4H), 2.54-2.49 (m, 2H), 2.39 (br d, J=2.0 Hz, 2H). LC/MS method A: R_{f}=2.64 mins., (M+H)⁺=482, purity >95%.

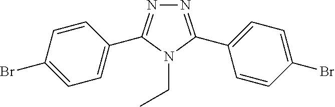 [SMILES: CCn1c(-c2ccc(Br)cc2)nnc1-c1ccc(Br)cc1]

Synthesis of 3,5-Bis(4-bromophenyl)-4-ethyl-4H-1,2,4-triazole: A flask was charged with 1020 mg (2.35 mmol) of ((Z)-4-bromo-N—((Z)-(4-bromophenyl)chloromethylene)benzohydrazonoyl chloride intermediate A), 540 mg (6.56 mmol) of ethylamine hydrochloride and 5 mL of methanol. Then 1060 mg (8.23 mmol) of diisopropylethylamine was added and reaction stirred for 30 minutes and heated at 65° C. for 18 hours. The reaction cooled and diluted with 5 mL methanol and 3 mL water added. After two hours solid collected and air dried for 18 hours to yield 730 mg of a solid (76%).

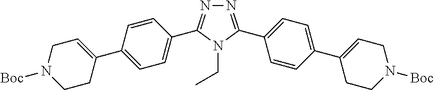 [SMILES: CCn1c(-c2ccc(C3=CCN(C(=O)OC(C)(C)C)CC3)cc2)nnc1-c1ccc(C2=CCN(C(=O)OC(C)(C)C)CC2)cc1]

Synthesis of tert-Butyl 4,4′-(4,4′-(4-ethyl-4H-1,2,4-triazole-3,5-diyl)bis(4,1-phenylene)) bis(5,6-dihydropyridine-1(2H)-carboxylate): A flask was charged with 310 mg (0.762 mmol) of 3,5-bis(4-bromophenyl)-4-ethyl-4H-1,2,4-triazole, 14 mg (0.061 mmol) of palladium acetate, 63 mg (0.152 mmol) of 2-dicyclohexylphosphino-2′,6′-dimethoxy biphenyl, 565 mg (1.83 mmol) of tert-butyl 4-(4,4,5,5-tetramethyl-1,3,2-dioxaborolan-2-yl)-5,6-dihydropyridine-1(2H)-carboxylate, 5 mL of dioxane and 1.3 mL of 2.0M K₂CO₃. Deoxygenate with nitrogen sparge for three minutes and heat to 95° C. for 18 hours. Reaction was cooled, diluted with 5 mL ethyl acetate and 5 mL water. After one hour solid was collected and air dried for one hour and reacted directly. Yield was 430 mg (92%).

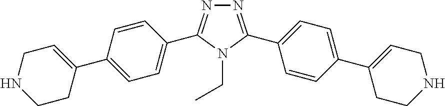 [SMILES: CCn1c(-c2ccc(C3=CCNCC3)cc2)nnc1-c1ccc(C2=CCNCC2)cc1]

Synthesis of 4,4′-(4,4′-(4-Ethyl-4H-1,2,4-triazole-3,5-diyl)bis(4,1-phenylene)) bis(1,2,3,6-tetrahydropyridine): A flask was charged with 430 mg (0.704 mmol) of tert-butyl 4,4′-(4,4′-(4-ethyl-4H-1,2,4-triazole-3,5-diyl)bis(4,1-phenylene))bis(5,6-dihydropyridine-1(2H)-carboxylate) was dissolved with a mixture of 3 mL dichloromethane and 8 mL trifluoro acetic acid (0.104 mmol) and stirred for 72 hours. The reaction was concentrated under vacuum and triturated with 20 mL of 4/1 ether/ethyl acetate. Yield was 520 mg of solid (82%).

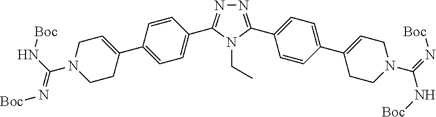 [SMILES: CCn1c(-c2ccc(C3=CCN(/C(=N\C(=O)OC(C)(C)C)NC(=O)OC(C)(C)C)CC3)cc2)nnc1-c1ccc(C2=CCN(/C(=N/C(=O)OC(C)(C)C)NC(=O)OC(C)(C)C)CC2)cc1]

Synthesis of tert-Butyl (4,4-(4,4-(4-ethyl-4H-1,2,4-triazole-3,5-diyl)bis(4,1-phenylene))bis(5,6-dihydropyridine-4,1(2H)-diyl))bis(methane-1-yl-1,1-diylidene) tetra carbamate prepared from 4,4′-(4,4′-(4-ethyl-4H-1,2,4-triazole-3,5-diyl)bis(4,1-phenylene)) bis(1,2,3,6-tetrahydropyridine): A flask was charged with 420 mg of 4,4′-(4,4′-(4-ethyl-4H-1,2,4-triazole-3,5-diyl)bis(4,1-phenylene))bis(1,2,3,6-tetrahydropyridine).bis trifluoroacetic acid salt was slurried with 4 mL N,N-dimethylformamide and 394 mg triethylamine added. After five minutes of stirring 504 mg (1.63 mmol) of tert-butyl (0.1H-pyrazol-1-yl)methane diylidenedicarbamate was added as a solid. The reaction was diluted with 40 mL ethyl acetate and washed with three 30 mL portions of water, dried and concentrated under vacuum. Yield was 220 mg (68%) after (20% methanol/dichloromethane)/dichloromethane silica column.

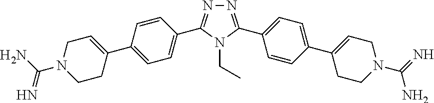 [SMILES: CCn1c(-c2ccc(C3=CCN(C(=N)N)CC3)cc2)nnc1-c1ccc(C2=CCN(C(=N)N)CC2)cc1]

### Example 2

Synthesis of 4,4′-(4,4′-(4-Ethyl-4H-1,2,4-triazole-3,5-diyl)bis(4,1-phenylene)) bis(5,6-dihydropyridine-1(2H)-carboximidamide): A flask was charged with 220 mg (0.246 mmol) of tert-Butyl (4,4′-(4,4′-(4-ethyl-4H-1,2,4-triazole-3,5-diyl)bis(4,1-phenylene))bis(5,6-dihydropyridine-4,1(2H)-diyl))bis(methane-1-yl-1,1-diylidene)tetracarbamate was dissolved with a mixture of 2 mL dichloromethane and 4 mL (51.9 mmol) of trifluoroacetic acid for 18 hours. The reaction was concentrated under vacuum, dissolved with N,N-dimethyl formamide and purified by reverse HPLC. Yield was 44 mg of product (25% yield). ¹H NMR (300 MHz, DMSO-d₆) δ=7.77-7.66 (m, 3H), 7.70 (d, J=4.1 Hz, 4H), 7.47 (s, 7H), 6.40-6.35 (m, 2H), 4.20-3.90 (m, 2H), 3.79-3.47 (m, 8H), 2.64 (br d, J=7.9 Hz, 2H), 0.95 (t, J=7.2 Hz, 3H). LC/MS method A: R_{f}=2.46 mins., (M+H)⁺=496,610, purity >95%.

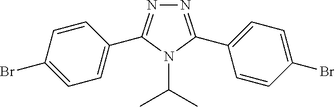 [SMILES: CC(C)n1c(-c2ccc(Br)cc2)nnc1-c1ccc(Br)cc1]

Synthesis of 3,5-Bis(4-bromophenyl)-4-isopropyl-4H-1,2,4-triazole: A flask was charged with 1020 mg (2.38 mmol) of (1,N)-4-Bromo-N-((4-bromophenyl)chloro methylene)benzohydrazonoyl chloride, 5 mL of methanol and 1050 mg (18.0 mmol) of isopropylamine. After 30 minutes of stirring at room temp the reaction was heated to 65° C. for 18 hours. The methanol was removed under vacuum and reaction heated at 95° C. for 24 hours and then 125° C. for 18 hours. Then 5 mL N-methyl-2-pyrrolidone added and reaction heated to 145° C. for 18 hours, 165° C. for six hours and 185° C. for 18 hours. After cooling, the reaction was diluted with 10 mL of water, aged for several hour and solid was collected and air dried in hood for several hours. Yield was 1120 mg (quantitative) of still wet solid which was used without further purification.

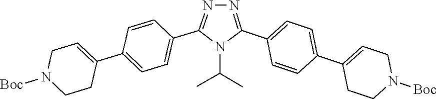 [SMILES: CC(C)n1c(-c2ccc(C3=CCN(C(=O)OC(C)(C)C)CC3)cc2)nnc1-c1ccc(C2=CCN(C(=O)OC(C)(C)C)CC2)cc1]

Synthesis of tert-Butyl 4,4′-(4,4′-(4-isopropyl-4H-1,2,4-triazole-3,5-diyl)bis (4,1-phenylene))bis(5,6-dihydropyridine-1(2H)-carboxylate): A flask was charged with 503 mg (1.19 mmol) of tert-butyl (4,4′-(4,4′-(4-isopropyl-4H-1,2,4-triazole-3,5-diyl)bis (4,1-phenylene))bis(5,6-dihydropyridine-4,1(2H)-diyl))bis(methane-1-yl-1,1-diylidene)\tetra carbamate, 22 mg (0.096 mmol) of palladium acetate, 98 mg (0.152 mmol) of 2-dicyclohexyl phosphino-2′,6′-dimethoxybiphenyl, 845 mg (274 mmol) of tert-butyl 4-(4,4,5,5-tetramethyl-1,3,2-dioxaborolan-2-yl)-5,6-dihydropyridine-1(2H)-carboxylate, 8 mL of dioxane and 4.2 mL of 2.0 M K₂CO₃. Deoxygenate with nitrogen sparge and heat to 95° C. for 18 hours. Reaction was cooled, diluted with 6 mL ethyl acetate and 10-mL water. Solid collected, rinsed with 2 mL, ethyl acetate and air dried in hood for 18 hours. Yield was 480 mg (64%).

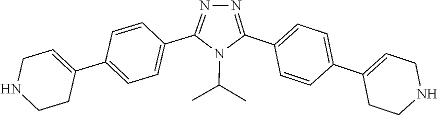 [SMILES: CC(C)n1c(-c2ccc(C3=CCNCC3)cc2)nnc1-c1ccc(C2=CCNCC2)cc1]

Synthesis of 4,4′-(4,4′-(4-Isopropyl-4H-1,2,4-triazole-3,5-diyl)bis(4,1-phenylene)) bis(1,2,3,6-tetrahydropyridine): A flask was charged with 480 mg (0.767 mmol) of tert-butyl 4,4′-(4,4′-(4-isopropyl-4H-1,2,4-triazole-3,5-diyl)bis(4,1-phenylene))bis(5,6-dihydropyridine-1(2H)-carboxylate and dissolved with a mixture of 4 mL dichloromethane and 500 mg (4.6 mmol) of anisole and 6 mL (77.9 mmol) of trifluoroacetic acid was added dropwise over several minutes and stirred for three hours. The reaction was concentrated under vacuum and triturated with two 20 mL portions of ether. Yield was quantitative.

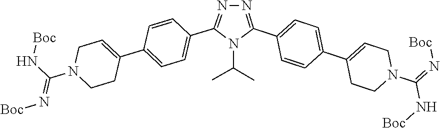 [SMILES: CC(C)n1c(-c2ccc(C3=CCN(/C(=N\C(=O)OC(C)(C)C)NC(=O)OC(C)(C)C)CC3)cc2)nnc1-c1ccc(C2=CCN(/C(=N/C(=O)OC(C)(C)C)NC(=O)OC(C)(C)C)CC2)cc1]

Synthesis of tert-Butyl (4,4′-(4,4′-(4-isopropyl-4H-1,2,4-triazole-3,5-diyl)bis (4,1-phenylene))bis(5,6-dihydropyridine-4,1(2H)-diyl))bis(methane-1-yl-1,1-diylidene) tetracarbamate: A flask was charged with 650 mg (0.70 mmol) of tert-Butyl (4,4′-(4,4′-(4-isopropyl-4H-1,2,4-triazole-3,5-diyl)bis(4,1-phenylene))bis(5,6-dihydropyridine-4,1(2H)-diyl))bis(methane-1-yl-1,1-diylidene)tetracarbamate bis trifluoroacetic acid salt was slurried with 4 mL N,N-dimethylformamide and 424 mg (4.20 mmol) of triethylamine added. After five minutes of stirring 521 mg (1.68 mmol) of tert-butyl (1H-pyrazol-1-yl)methane diylidenedicarbamate was added as a solid. After stirring for 18 hours at 23° C., the reaction was diluted with 30 mL ethyl acetate, washed with three×20 mL of water, dried and concentrated under vacuum. Yield was quantitative.

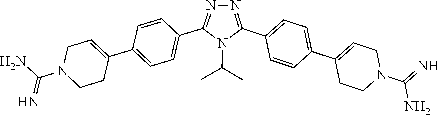 [SMILES: CC(C)n1c(-c2ccc(C3=CCN(C(=N)N)CC3)cc2)nnc1-c1ccc(C2=CCN(C(=N)N)CC2)cc1]

### Example 3

Synthesis of 4,4′-(4,4′-(4-isopropyl-4H-1,2,4-triazole-3,5-diyl)bis(4,1-phenylene))bis(5,6-dihydropyridine-1 (2H)-carboximidamide): A round bottom flask was charged with 270 mg of tert-butyl (4,4-(4,4′-(4-isopropyl-4H-1,2,4-triazole-3,5-diyl)bis(4,1-phenylene))bis (5,6-dihydropyridine-4,1(2H)-diyl))bis(methane-1-yl-1,1-diylidene)tetracarbamate, 4 mL dichloromethane, 400 mg (3.7 mmol) of anisole and 4 mL (51.9 mmol) of trifluoroacetic acid added over several minutes. After 3 hours the reaction was concentrated under vacuum, triturated with two 20 mL portions of ether, dissolved with N,N-dimethylformamide and purified by reverse phase prep HPLC. Yield was 260 mg (50%). ¹H NMR (300 MHz, DMSO-d₆) δ=7.69-7.58 (m, 8H), 7.50 (s, 6H), 6.37 (br t, J=3.2 Hz, 2H), 4.44-4.06 (m, 6H), 3.65 (t, J=5.5 Hz, 4H), 2.65 (br t, J==5.1 Hz, 2H), 1.21 (d, J=6.9 Hz, 6H). LC/MS method A: R_{f}=2.58 mins., (M+H)⁺=255,510, purity >95%.

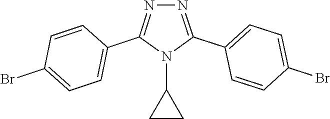 [SMILES: Brc1ccc(-c2nnc(-c3ccc(Br)cc3)n2C2CC2)cc1]

Synthesis of 3,5-bis(4-bromophenyl)-4-cyclopropyl-4H-1,2,4-triazole: A flask was charged with (Z)-4-bromo-N—((Z)-(4-bromophenyl)chloromethylene)benzohydrazonoyl chloride (1.0 grams, 2.3 mmol) was slurried with 5 mL of methanol and the cyclopropylamine (0.4 g, 7.5 mmol), 2.0 M in methanol was added over five minutes followed by 0.20 grams (3.45 mmol) of glacial acetic acid was added over five minutes. The reaction was heated at 70° C. for 18 hours. The reaction was allowed to cool and most of methanol removed under vacuum. Then 40 mL of water added over several minutes. One hour later the solid was collected, air dried for 15 minutes and rinsed with ten mL of water, ten mL of ether and air dried in the hood for several hours. Yield was 0.776 grams (80%).

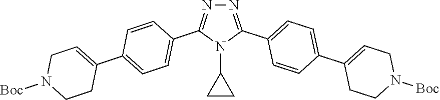 [SMILES: CC(C)(C)OC(=O)N1CC=C(c2ccc(-c3nnc(-c4ccc(C5=CCN(C(=O)OC(C)(C)C)CC5)cc4)n3C3CC3)cc2)CC1]

Synthesis of tert-Butyl 4,4′-(4,4′-(4-cyclopropyl-4H-1,2,4-triazole-3,5-diyl)bis(4,1-phenylene))bis(5,6-dihydropyridine-1(2H)-carboxylate): A flask was charged with 300 mg (0.71 mmol) of 3,5-bis(4-bromophenyl)-4-cyclopropyl-4H-1,2,4-triazole, 12.8 mg (0.05 mmol) of palladium acetate, 58 mg (0.14 mmol) of 2-dicyclohexylphosphino-2′,6′-dimethoxybiphenyl, 531 mg (1.7.1 mmol) of tert-butyl 4-(4,4,5,5-tetramethyl-1,3,2-dioxaborolan-2-yl)-5,6-dihydropyridine-1(2H)-carboxylate, 2.0 of 1,4-dioxane and 1.0 mL, of 2.0 molar potassium carbonate aqueous solution. The flask was swept with nitrogen for five minutes and heated at 95° C. for 18 hours. Then the reaction was complete by LCMS and the reaction was allowed to cool and diluted with 4 mL water and 10 mL of ethyl acetate added over ten minutes. Solids formed quickly and reaction stirred for two hours. Solid collected on a filter and rinsed with 30 mL ethyl acetate and air-dried for 18 hours in the hood to yield 222 mg (49%).

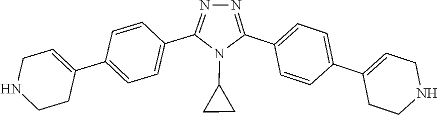 [SMILES: C1=C(c2ccc(-c3nnc(-c4ccc(C5=CCNCC5)cc4)n3C3CC3)cc2)CCNC1]

Synthesis of 4,4′-(4,4′-(4-Cyclopropyl-4H-1,2,4-triazole-3,5-diyl)bis(4,1-phenylene)) bis(1,2,3,6-tetrahydropyridine) Di-tert-butyl 4,4′-((4-cyclopropyl-4H-1,2,4-triazole-3,5-diyl)bis(4,1-phenylene))bis(5,6-dihydropyridine-1(2H)-carboxylate) 200 mg (0.32 mmol) was slurried with 0.5 mL of anisole and 1.5 mL dichloromethane. Then 1.0 mL (40 mmol) of trifluoroacetic acid added over five minutes with stirring. The reaction was concentrated under vacuum and triturated with two 5 mL portions of ether and ether decanted away to yield a solid in quantitative yield.

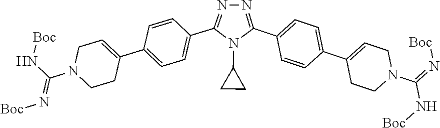 [SMILES: CC(C)(C)OC(=O)/N=C(/NC(=O)OC(C)(C)C)N1CC=C(c2ccc(-c3nnc(-c4ccc(C5=CCN(/C(=N/C(=O)OC(C)(C)C)NC(=O)OC(C)(C)C)CC5)cc4)n3C3CC3)cc2)CC1]

Synthesis of tert-Butyl (4,4-(4,4-(4-cyclopropyl-4H-1,2,4-triazole-3,5-diyl)bis(4,1-phenylene))bis(5,6-dihydropyridine-4,1(2H)-diyl))bis(methane-1-yl-1,1-diylidene) tetracarbamate: A flask was charged with 100 mg (0.23 mmol) of 4,4′-((4-cyclopropyl-4H-1,2,4-triazole-3,5-diyl)bis(4,1-phenylene))bis(1,2,3,6-tetrahydropyridine) bis trifluoroacetic acid salt and 1.0 mL N,N-dimethylformamide. Then 143 mg (1.41 mmol) of triethylamine added. Then 183.1 mg (0.54 mmol) of tert-butyl (1H-pyrazol-1-yl)methanediylidenedicarbamate was added. The reaction slurry was warmed at 40° C. for 18 hours. The reaction was diluted with 20 mL ethyl acetate and then washed with four portions of 5 mL of water. Organic phase was dried with sodium sulfate and concentrated under vacuum. The product was purified by normal phase chromatography, loaded as dichloromethane solution, eluted with (30% acetonitrile/dichloromethane)/dichloromethane with step gradient from 0 to 100% to elute impurities. Then the expected product eluted off with (20% methanol/dichloromethane)/ dichloromethane step gradient from 0 to 100%. Yield was 36.3 mg of product (17%).

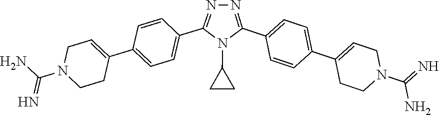 [SMILES: N=C(N)N1CC=C(c2ccc(-c3nnc(-c4ccc(C5=CCN(C(=N)N)CC5)cc4)n3C3CC3)cc2)CC1]

### Example 4

Synthesis of 4,4′-(4,4′-(4-Cyclopropyl-4H-1,2,4-triazole-3,5-diyl)bis(4,1-phenylene)) bis(5,6-dihydropyridine-1(2H)-carboximidamide): A solution of di-tert-butyl ((4,4′-((4-cyclopropyl-4H-1,2,4-triazole-3,5-diyl)bis(4,1-phenylene))bis(5,6-dihydropyridine-4,1(2H)-diyl))bis (((tert-butoxycarbonyl)amino)methanylylidene)) dicarbamate (36 mg, 0.04 mmol) was dissolved with a mixture of 0.5 mL of anisole and 4 mL of dichloromethane. Then 2 mL of trifluoroacetic acid added over two minutes with stirring and stirred for 18 hours. The next day LCMS indicted reaction complete and reaction concentrated under vacuum. Oil was triturated with two 0.10 mL portions of ether and ether decanted away to yield a solid product. Yield was 9.4 mg (46%). ¹H NMR (300 MHz, DMSO-d₆) δ=7.86-8.00 (m, 4H) 7.69 (s, 4H) 7.44 (br. s., 6H) 6.34-6.46 (m, 2H) 4.06-4.18 (m, 4H) 3.94-4.03 (m, 1H) 2.54-2.82 (m, 4H) 0.78-0.93 (m, 2H) 0.18-0.37 (m, 2H); LC/MS method A: R_{f}=3.05 mins., (M+H)⁺=508, purity >95%.

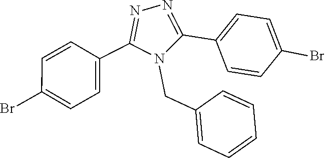 [SMILES: Brc1ccc(-c2nnc(-c3ccc(Br)cc3)n2Cc2ccccc2)cc1]

Synthesis of 4-Benzyl-3,5-bis(4-bromophenyl)-4H-1,2,4-triazole: A flask was charged with the crude (Z)-4-bromo-N—((Z)-(4-bromophenyl)chloromethylene)benzo hydrazonoyl chloride (1.0 grams, 2.12 mmol) was slurried with 5 mL of methanol and the benzylamine (0.75 g, 7.01 mmol) was added over five minutes followed by 0.19 grams (3.19 mmol) of glacial acetic acid was added over five minutes. The reaction was heated at 70° C. for 18 hours. The reaction was allowed to cool and most of methanol removed under vacuum. Then 40 mL of water added over several minutes. One hour later the solid was collected, air dried for 15 minutes and rinsed with ten mL of water, ten mL of ether and air dried in the hood. Yield was 1.01 grams (94%).

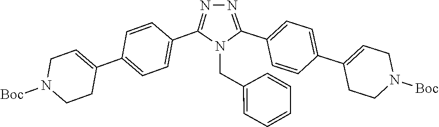 [SMILES: CC(C)(C)OC(=O)N1CC=C(c2ccc(-c3nnc(-c4ccc(C5=CCN(C(=O)OC(C)(C)C)CC5)cc4)n3Cc3ccccc3)cc2)CC1]

Synthesis of tert-Butyl 4,4′-(4,4′-(4-benzyl-4H-1,2,4-triazole-3,5-diyl)bis(4,1-phenylene)) bis(5,6-dihydropyridine-1(2H)-carboxylate): A flask was charged with 300 mg (0.59 mmol) of 4-benzyl-3,5-bis(4-bromophenyl)-4H-1,2,4-triazole, 10.6 mg (0.04 mmol) of palladium acetate, 48.7 mg (0.11 mmol) of 2-dicyclohexylphosphino-2′,6′-dimethoxybiphenyl, 440 mg (1.42 mmol) of tert-butyl 4-(4,4,5,5-tetramethyl-1,3,2-dioxaborolan-2-yl)-5,6-dihydropyridine-1(2H)-carboxylate, 2.0 mL of 1,4-dioxane and 1.0 mL of 2.0 molar potassium carbonate aqueous solution. The flask was swept with nitrogen for five minutes and heated at 95° C. for 18 hours. Then the reaction was complete by LCMS and the reaction was allowed to cool and diluted with 4 mL water and 10 mL of ethyl acetate added over ten minutes. Solids formed quickly and reaction stirred for two hours. Solid collected on a filter and rinsed with 30 mL ethyl acetate and air-dried in the hood to yield 211 mg (50%).

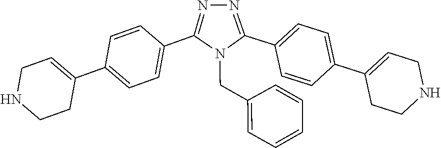 [SMILES: C1=C(c2ccc(-c3nnc(-c4ccc(C5=CCNCC5)cc4)n3Cc3ccccc3)cc2)CCNC1]

Synthesis of 4,4′-(4,4′-(4-Benzyl-4H-1,2,4-triazole-3,5-diyl)bis(4,1-phenylene))bis(1,2,3,6-tetrahydropyridine: Di-tert-butyl 4,4′-((4-benzyl-4H-1,2,4-triazole-3,5-diyl)bis(4,1-phenylene))bis(5,6-dihydropyridine-1(2H)-carboxylate) 200 mg (0.28 mmol) was slurried with 0.5 mL of anisole and 1.5 mL dichloromethane. Then 1.0 mL (40 mmol) of trifluoroacetic acid added over five minutes with stirring. The reaction was concentrated under vacuum and triturated with two 5 mL portions of ether and ether decanted away to yield a solid in quantitative yield.

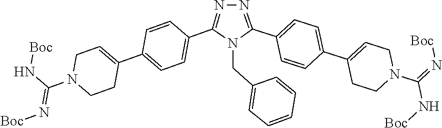 [SMILES: CC(C)(C)OC(=O)/N=C(/NC(=O)OC(C)(C)C)N1CC=C(c2ccc(-c3nnc(-c4ccc(C5=CCN(/C(=N/C(=O)OC(C)(C)C)NC(=O)OC(C)(C)C)CC5)cc4)n3Cc3ccccc3)cc2)CC1]

Synthesis of tert-Butyl (4,4′-(4,4′-(4-benzyl-4H-1,2,4-triazole-3,5-diyl)bis(4,1-phenylene)) bis(5,6-dihydropyridine-4,1(2H)-diyl))bis(methane-1-yl-1,1-diylidene)tetracarbamate: A flask was charged with 100 mg (0.19 mmol) of 4,4′4-(4-benzyl-4H-1,2,4-triazole-3,5-diyl)bis(4,1-phenylene))bis(1,2,3,6-tetrahydropyridine) bis trifluoroacetic acid salt and 1.0 mL N,N-dimethylformamide. Then 119 mg (1.17 mmol) of triethylamine added. Then 152 mg (0.49 mmol) of fret-butyl (1H-pyrazol-1-yl)methanediylidenedicarbamate was added. The reaction slurry was warmed at 40° C. for 18 hours. The reaction was diluted with 20 mL ethyl acetate and then washed with four portions of 5 mL of water. Organic phase was dried with sodium sulfate and concentrated under vacuum. The product was purified by normal phase chromatography, loaded as dichloromethane solution, eluted with (30% acetonitrile/dichloromethane)/dichloromethane with step gradient from 0 to 100% to elute impurities. Then the expected product eluted off with (20% MeOH/dichloromethane)/dichloromethane step gradient from 0 to 100%. Yield was 26.6 mg of product (14%).

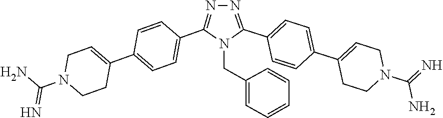 [SMILES: N=C(N)N1CC=C(c2ccc(-c3nnc(-c4ccc(C5=CCN(C(=N)N)CC5)cc4)n3Cc3ccccc3)cc2)CC1]

### Example 5

Synthesis of 4,4′-(4,4′-(4-Benzyl-4H-1,2,4-triazole-3,5-diyl)bis(4,1-phenylene))bis(5,6-dihydropyridine-1(2H)-carboximidamide): A solution of di-tert-butyl ((4,4′-((4-benzyl-4H-1,2,4-triazole-3,5-diyl)bis(4,1-phenylene))bis(5,6-dihydropyridine-4,1(2H)-diyl))bis (((tert-butoxycarbonyl)amino)methanylylidene)) dicarbamate (29 mg, 0.030 mmol) was dissolved with a mixture of 0.5 mL of anisole and 1.5 mL of dichloromethane. Then 2 mL of trifluoroacetic acid added over two minutes with stirring and stirred for 18 hours. The next day LCMS indicted reaction complete and reaction concentrated under vacuum. Oil was triturated with two 5 mL portions of ether and ether decanted away to yield a solid product. Yield was 13.2 mg (78%). ¹H NMR (300 MHz, DMSO-d₆) − ppm 7.61 (d, J=5.27 Hz, 9H) 7.41 (s, 8H) 7.13-7.31 (m, 2H) 6.72-6.90 (m, 2H) 6.26-6.43 (m, 2H) 5.33-5.49 (m, 2H) 4.02-4.19 (m, 4H) 2.55-2.65 (m, 4H); LC/MS method A: R_{f}=3.32 mins., (M+H)⁺=558, purity >95%.

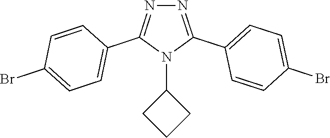 [SMILES: Brc1ccc(-c2nnc(-c3ccc(Br)cc3)n2C2CCC2)cc1]

Synthesis of 3,5-Bis(4-bromophenyl)-4-cyclobutyl-4H-1,2,4-triazole: A flask was charged with 1260 mg (2.92 mmol) of (Z)-4-bromo-N—((Z)-(4-bromophenyl)chloromethylene)benzohydrazonoyl chloride and 4 mL of methanol and 4 N,N-dimethyl formamide. Then 477 mg (6.72 mmol) of cyclobutylamine was added dropwise over one minute. Then 3 minutes later 1170 mg (8.76 mmol) of diisopropylethylamine was added at once and reaction stirred for ten minutes and heated at 65° C. for 72 hours. The reaction cooled and methanol removed under vacuum and 5 mL (83.3 mmol) of glacial acetic acid added and reaction heated at 95° C. for 18 hours. The reaction was cooled and diluted with 40 mL of water and stirred for one hour. The solid was collected, rinsed with 10 mL of water, 10 mL of ether and air dried for one hour. Yield was 1000 rig of product (79%).

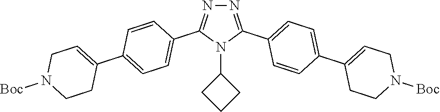 [SMILES: CC(C)(C)OC(=O)N1CC=C(c2ccc(-c3nnc(-c4ccc(C5=CCN(C(=O)OC(C)(C)C)CC5)cc4)n3C3CCC3)cc2)CC1]

Synthesis of tert-Butyl 4,4′-(4,4′-(4-cyclobutyl-4H-1,2,4-triazole-3,5-diyl)bis(4,1-phenylene))bis(5,6-dihydropyridine-1(2H)-carboxylate): A flask was charged with 570 mg (1.32 mmol) of 3,5-Bis(4-bromophenyl)-4-cyclobutyl-4H-1,2,4-triazole, 24 mg (0.106 mmol) of palladium acetate, 108 mg (0.264 mmol) of 2-dicyclohexylphosphino-4′,6′-dimethoxybiphenyl, 936 mg (3.03 mmol) of tert-butyl 4-(4,4,5,5-tetramethyl-1,3,2-dioxaborolan-2-yl)-5,6-dihydropyridine-1(2H)-carboxylate, 8 mL of 1,4-dioxane and 5.0 mL of 2.0 molar potassium carbonate aqueous solution. The flask was swept with nitrogen for five minutes and heated at 95° C. for 18 hours. The reaction was complete by LCMS and the reaction was allowed to cool and diluted with 5 mL water and 10 mL of ethyl acetate. Solids formed quickly and reaction stirred for 90 minutes. Solid collected on a filter and rinsed with 5 mL ethyl acetate and 10 mL ether and air-dried for 18 hours in the hood to yield 698 mg (83%) of product.

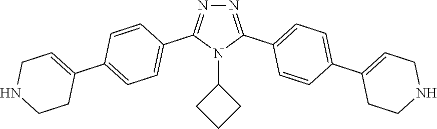 [SMILES: C1=C(c2ccc(-c3nnc(-c4ccc(C5=CCNCC5)cc4)n3C3CCC3)cc2)CCNC1]

Synthesis of 4,4′-(4,4′-(4-Cyclobutyl-4H-1,2,4-triazole-3,5-diyl)bis(4,1-phenylene)) bis(1,2,3,6-tetrahydropyridine): A flask was charged with tert-butyl 4,4′-(4,4′-(4-cyclobutyl-4H-1,2,4-triazole-3,5-diyl)bis(4,1-phenylene))bis(5,6-dihydropyridine-1(2H)-carboxylate) was slurried with 700 mg (6.48 mmol) of anisole and 5 mL dichloromethane. Then 7 mL (90.9 mmol) of trifluoroacetic acid added over five minutes with stirring. Three hours later the reaction was concentrated under vacuum and triturated with two 25 mL portions of ether and ether decanted away to yield a solid in quantitative yield.

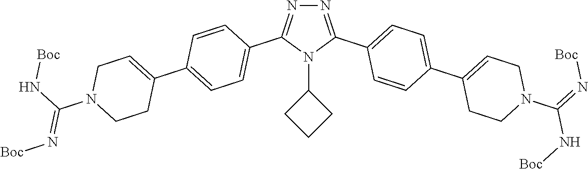 [SMILES: CC(C)(C)OC(=O)/N=C(/NC(=O)OC(C)(C)C)N1CC=C(c2ccc(-c3nnc(-c4ccc(C5=CCN(/C(=N/C(=O)OC(C)(C)C)NC(=O)OC(C)(C)C)CC5)cc4)n3C3CCC3)cc2)CC1]

Synthesis of tert-Butyl (4,4′-(4,4′-(4-cyclobutyl-4H-1,2,4-triazole-3,5-diyl)bis(4,1-phenylene))bis(5,6-dihydropyridine-4,1(2H)-diyl))bis(methane-1-yl-1,1-diylidene) tetracarbamate: A flask was charged with 710 mg (0.95 mmol) of 4,4′-(4,4′-(4-cyclobutyl-4H-1,2,4-triazole-3,5-diyl)bis(4,1-phenylene))bis(1,2,3,6-tetrahydropyridine) bis trifluoroacetic, 4 mL N,N-dimethylformamide and 1 mL dichloromethane. Then 672 mg (6.65 mmol) of triethylamine added over two minutes. Then 736 mg (2.38 mmol) of tert-butyl (1H-pyrazol-1-yl)methanediylidenedicarbamate was added at once. The reaction slurry was warmed at 45° C. for six hours. The reaction allowed to cool and an additional 100 mg (0.32 mmol) of tert-butyl (1H-pyrazol-1-yl)methanediylidenedicarbamate was added. After 24 hours, the reaction was diluted with 25 mL ethyl acetate and then washed with three 20 mL of water. Organic phase was dried with sodium sulfate and concentrated under vacuum to yield 950 mg of an oil in a quantitative yield.

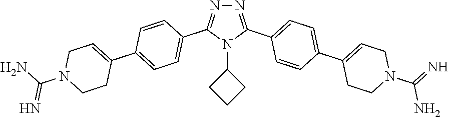 [SMILES: N=C(N)N1CC=C(c2ccc(-c3nnc(-c4ccc(C5=CCN(C(=N)N)CC5)cc4)n3C3CCC3)cc2)CC1]

### Example 6

Synthesis of 4,4′-(4,4′-(4-Cyclobutyl-4H-1,2,4-triazole-3,5-diyl)bis(4,1-phenylene))bis(5,6-dihydropyridine-1(2H)-carboximidamide): A flask was charged with 950 mg (0.95 mmol) of tert-butyl (4,4′-(4,4′-(4-cyclobutyl-4H-1,2,4-triazole-3,5-diyl)bis(4,1-phenylene)) bis(5,6-dihydropyridine-4,1(2H)-diyl))bis(methane-1-yl-1,1-diylidene)tetracarbamate and 4 mL dichloromethane. Then 6 mL (77.9 mmol) of trifluoroacetic acid was added over five minutes with stirring. Two hours later LCMS indicted reaction complete and reaction concentrated under vacuum. Oil was triturated with two 50 mL portions of ether and ether decanted away to yield a solid. Solid was dissolved in N,N-dimethylformamide and was purified by prep HPLC. Yield was 181 mg (25%). ¹H NMR (300 MHz, DMSO-d₆) δ=7.73-7.62 (m, 7H), 7.51-7.36 (m, 7H), 6.39-6.35 (m, 2H), 5.25-5.15 (m, 1H), 4.14-4.08 (m, 2H), 3.68-3.61 (m, 2H), 3.53-3.45 (m, 2H), 3.32-3.20 (m, 2H), 2.72-2.61 (m, 4H), 1.91-1.81 (m, 2H), 1.68-1.43 (m, 2H), 1.43-1.35 (m, 2H). LC/MS method A: R_{f}=3.43 mins., (M+H)⁺=523, purity >95%.

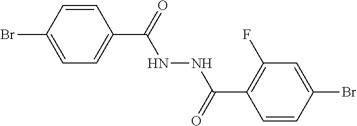 [SMILES: O=C(NNC(=O)c1ccc(Br)cc1F)c1ccc(Br)cc1]

Synthesis of 4-Bromo-N′-(4-bromobenzoyl)-2-fluorobenzohydrazide: A 250 mL round bottom flask was charged with 1,840 mg (8.5 mmol) of 4-bromobenzohydrazide, 1540 mg of N. N-diisopropylethylamine (12.0 mmol) and 15 mL of chloroform and reaction slurry was cooled under ice water bath. To the reaction slurry added 2020 mg (8.54 mmol) of 4-bromo-3-fluorobenzoyl chloride in small portions over one hour. The cooling bath was removed and reaction was stirred 18 hours at 23° C. The reaction mixture was concentrated under vacuum. The solid was sonicated with 30.0 mL of water for five minutes and stirred for one hour. The water was decanted and briefly triturated with 10 mL of methanol. The solvent was decanted and evaporated under reduced pressure. To solid compound added 100 mL of acetonitrile and removed under vacuum. Process repeated twice and dried under vacuum to obtain product which was used in the next step without further purification.

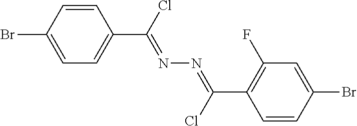 [SMILES: Fc1cc(Br)ccc1/C(Cl)=N/N=C(\Cl)c1ccc(Br)cc1]

Synthesis of (Z)-4-Bromo-N-[(1Z)-(4-bromophenyl)(chloro)methylidene]-2-fluorobenzene-1-carbohydrazonoyl chloride: A 250 mL flask was charged with 4 g (9.8 mmol) of 4-bromo-N-(4-bromobenzoyl)-3-fluorobenzohydrazide. The solid was slurried with 50 mL toluene and briefly sonicated. 6.12 g (208 mmol) of PCl₅ was added in three roughly equal portions. The reaction was heated to 100° C. for 18 hours. The reaction was cooled and concentrated under vacuum to yield a solid. The reaction was quenched by addition of wet ice and water (about 40 grams). After a two-hour age water removed by decantation and two 50 mL portions of acetonitrile added and removed under vacuum to remove water. Yield 3.3 grams of solid (74%).

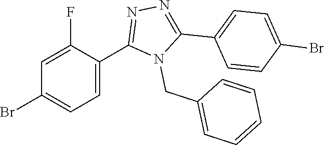 [SMILES: Fc1cc(Br)ccc1-c1nnc(-c2ccc(Br)cc2)n1Cc1ccccc1]

Synthesis of 4-Benzyl-3-(4-bromo-2-fluorophenyl)-5-(4-bromophenyl)-4H-1,2,4-triazole: A flask was charged with (Z)-4-bromo-N-[(1Z)-(4-bromophenyl)(chloro)methylidene]-2-fluorobenzene-1-carbohydrazonoyl chloride (500 mg, 1.14 mmol) was slurried with 5 mL of methanol and the benzylamine (0.4 g, 3.79 mmol), was added over five minutes followed by 103 mg (1.72 mmol) of glacial acetic acid was added over five minutes. The reaction was heated at 70° C. for 18 hours. The reaction was allowed to cool and most of methanol removed under vacuum. Then 40 mL of water added over several minutes. One hour later the solid was collected, air dried for 15 minutes and rinsed with ten mL of water, ten mL of ether and air dried in the hood for several hours. Yield was 0.4 grams (74%).

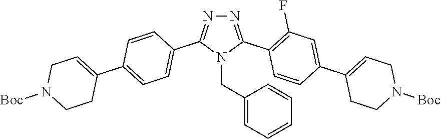 [SMILES: CC(C)(C)OC(=O)N1CC=C(c2ccc(-c3nnc(-c4ccc(C5=CCN(C(=O)OC(C)(C)C)CC5)cc4F)n3Cc3ccccc3)cc2)CC1]

Synthesis of tert-Butyl 4-(4-(4-benzyl-5-(4-(1-(tert-butoxycarbonyl)-1,2,3,6-tetrahydro pyridin-4-yl)-2-fluorophenyl)-4H-1,2,4-triazol-3-yl)phenyl)-5,6-dihydropyridine-1(2H)-carboxylate): A flask was charged with 400 mg (0.82 mmol) of 4-benzyl-3-(4-bromo-2-fluorophenyl)-5-(4-bromophenyl)-4H-1,2,4-triazole 14.7 mg (0.06 mmol) of palladium acetate, 67 mg (0.16 mmol) of 2-dicyclohexylphosphino-2′,6′-dimethoxybiphenyl, 609 mg (1.97 mmol) of tert-butyl 4-(4,4,5,5-tetramethyl-1,3,2-dioxaborolan-2-yl)-5,6-dihydropyridine-1(2H)-carboxylate, 2.0 mL of 1,4-dioxane and 1.0 mL of 2.0 molar potassium carbonate aqueous solution. The flask was swept with nitrogen for five minutes and heated at 95° C. for 18 hours. Then the reaction was complete by LCMS and the reaction was allowed to cool and diluted with 4 mL water and 10 mL of ethyl acetate added over ten minutes. Solids formed quickly and reaction stirred for two hours. Solid collected on a filter and rinsed with 30 mL, ethyl acetate and air-dried in the hood to yield 244 mg (43%).

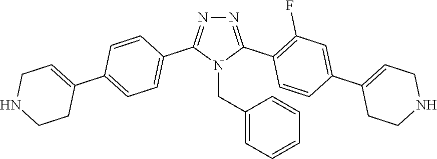 [SMILES: Fc1cc(C2=CCNCC2)ccc1-c1nnc(-c2ccc(C3=CCNCC3)cc2)n1Cc1ccccc1]

Synthesis of 4-(4-(4-Benzyl-5-(2-fluoro-4-(1,2,3,6-tetrahydropyridin-4-yl)phenyl)-4H-1,2,4-triazol-3-yl)phenyl)-1,2,3,6-tetrahydropyridine: tert-butyl 4-(4-(4-benzyl-5-(4-(1-(tert-butoxycarbonyl)-1,2,3,6-tetrahydropyridin-4-yl)-2-fluorophenyl)-4H-1,2,4-triazol-3-yl)phenyl)-5,6-dihydropyridine-1(2H)-carboxylate 244 mg (0.35 mmol) was slurried with 0.5 mL of anisole and 1.5 mL dichloromethane. Then 1.0 mL (13 mmol) of trifluoroacetic acid added over five minutes with stirring. The reaction was concentrated under vacuum and triturated with two 5 mL portions of ether and ether decanted away to yield a solid in quantitative yield.

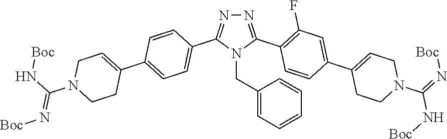 [SMILES: CC(C)(C)OC(=O)/N=C(/NC(=O)OC(C)(C)C)N1CC=C(c2ccc(-c3nnc(-c4ccc(C5=CCN(/C(=N/C(=O)OC(C)(C)C)NC(=O)OC(C)(C)C)CC5)cc4)n3Cc3ccccc3)c(F)c2)CC1]

Synthesis of tert-Butyl N-[(E)-(4-{4-[4-benzyl-5-(4-{1-[(Z)-{[(tert-butoxy)carbonyl]amino}-({[(tert-butoxy)carbonyl]imino})methyl]-1,2,3,6-tetrahydropyridin-4-yl}phenyl)-4H-1,2,4-triazol-3-yl]-3-fluorophenyl}-1,2,3,6-tetrahydropyridin-1-yl)({[(tert-butoxy)carbonyl] imino})methyl]carbamate: A flask was charged with 100 mg (0.21 mmol) of 4-(4-(4-benzyl-5-(2-fluoro-4-(1,2,3,6-tetrahydropyridin-4-yl)phenyl)-4H-1,2,4-triazol-3-yl)phenyl)-1,2,3,6-tetrahydropyridine bis trifluoroacetic acid salt and 1.0 mL N,N-dimethylformamide. Then 123 mg (1.22 mmol) of triethylamine added. Then 189 mg (0.61 mmol) of tert-butyl (1H-pyrazol-1-yl)methanediylidenedicarbamate was added. The reaction slurry was warmed at 40° C. for 18 hours. The reaction was diluted with 20 mL ethyl acetate and then washed with four portions of 5 mL, of water. Organic phase was dried with sodium sulfate and concentrated under vacuum. The product was purified by normal phase chromatography, loaded as dichloromethane solution, eluted with (30% acetonitrile/dichloromethane)/dichloromethane with step gradient from 0 to 100% to elute impurities. Then the expected product eluted off with (20% MeOH/dichloromethane)/dichloromethane step gradient from 0 to 100%. Yield was 81.5 mg of product (41%).

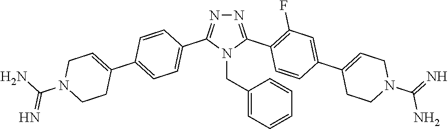 [SMILES: N=C(N)N1CC=C(c2ccc(-c3nnc(-c4ccc(C5=CCN(C(=N)N)CC5)cc4F)n3Cc3ccccc3)cc2)CC1]

### Example 7

Synthesis of 4-(4-(4-Benzyl-5-(4-(1-carbamimidoyl-1,2,3,6-tetrahydropyridin-4-yl)-2-fluorophenyl)-4H-1,2,4-triazol-3-yl)phenyl)-5,6-dihydropyridine-1(2H)-carboximidamide: A solution of di-tert-butyl ((4,4′-((4-benzyl-4H-1,2,4-triazole-3,5-diyl)(3-fluoro-4,1-phenylene)(4-phenylene))bis(5,6-dihydropyridine-4,1(2H)-diyl))bis(((tert-butoxycarbonyl)amino) methanylylidene))dicarbamate (81 mg, 0.08 mmol) was dissolved with a mixture of 0.5 mL of anisole and 4 mL of dichloromethane. Then 2 mL, of trifluoroacetic acid added over two minutes with stirring and stirred for 18 hours. The next day LCMS indicted reaction complete and reaction concentrated under vacuum. Oil was triturated with two 10 mL portions of ether and ether decanted away to yield a solid product. Yield was 65 mg (95%). ¹H NMR (300 MHz, DMSO-d₆) ppm 7.39-7.66 (m, 12H) 6.50 (br. s., 2H) 4.07-4.24 (m, 4H) 3.31-3.45 (m, 5H) 2.66 (br. s., 4H) 0.55-0.75 (m, 2H) 0.17-0.37 (m, 2H); LC/MS method A: R_{f}=3.10 mins., (M+H)⁺=544, purity >95%.

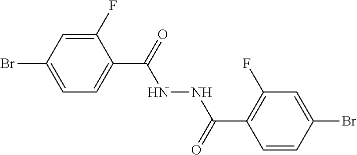 [SMILES: O=C(NNC(=O)c1ccc(Br)cc1F)c1ccc(Br)cc1F]

Synthesis of 4-Bromo-N-(4-bromo-2-fluorobenzoyl)-2-fluorobenzohydrazide: A flask was charged with 50 mL of chloroform and cooled in an ice water bath and 1.140 mg (45.7 mmol) of hydrazine monohydrate (7670 mg, 59.4 mmol) of diisopropylethylamine was added. A solution of 10800 mg (45.7 mmol) of 4-bromo-2-fluorobenzoyl chloride in 50 mL of chloroform was added over one hour and stirred for 18 hours at 23° C. Removed chloroform under vacuum and stir with 150 mL of water for 18 hours after brief sonication. Solids isolated by decantation/filtration. Filter cake/flask rinsed with 30 mL acetonitrile. Solids from filter added to solids from flask and two 120 mL, portions of acetonitrile added and removed under vacuum to dry solid. Yield was 9.53 grams (96%).

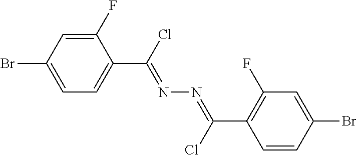 [SMILES: Fc1cc(Br)ccc1/C(Cl)=N/N=C(\Cl)c1ccc(Br)cc1F]

Synthesis of (Z)-4-bromo-N-[(1Z)-(4-bromo-2-fluorophenyl)(chloro) methylidene]-2-fluorobenzene-1-carbohydrazonoyl chloride: A 250 mL flask was charged with 9500 mg (22.0 mmol) of (4-bromo-N′-(4-bromo-2-fluorobenzoyl)-2-fluorobenzohydrazide. The solid was slurried with 100 mL toluene and briefly sonicated. 13700 mg (66.0 mmol) of PCl₅ was added in three roughly equal portions. The reaction was heated to 100° C. for 18 hours. The reaction was cooled and concentrated under vacuum to yield a solid. The reaction was quenched by addition of wet ice and water (about 50 grams). After a two-hour age water removed by decantation and two 100 mL portions of acetonitrile added and removed under vacuum to remove water. Yield 8.8 grams of solid (85%).

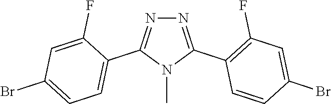 [SMILES: Cn1c(-c2ccc(Br)cc2F)nnc1-c1ccc(Br)cc1F]

Synthesis of 3,5-Bis(4-bromo-2-fluorophenyl)-4-methyl-4H-1,2,4-triazole: A flask was charged with the crude (Z)-4-bromo-N-[(1Z)-(4-bromo-2-fluorophenyl)(chloro)methylidene]-2-fluorobenzene-1-carbohydrazonoyl chloride (11.2 grams, 30 mmol) was slurried with 15 mL of methanol and the methylamine (37.5 mL, 75 mol), 2.0 M in methanol was added over ten minutes. Then 12.4 grams (96.0 mmol) of N,N-diisopropylethylamine was added over ten minutes. After 30 minutes the reaction was slowly warmed to 50° C. for two hours and reaction cooled and 15.0 grams (250 mmol) of glacial acetic acid was added over ten minutes. The reaction was heated at 80° C. for three hours. The reaction was allowed to cool and most of methanol removed under vacuum. Then 40 mL of water added over several minutes. One hour later the solid was collected, air dried for 15 minutes and rinsed with ten mL of ether and air dried in the hood for several hours. Yield was 9.42 grams (73%).

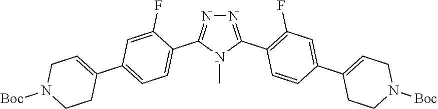 [SMILES: Cn1c(-c2ccc(C3=CCN(C(=O)OC(C)(C)C)CC3)cc2F)nnc1-c1ccc(C2=CCN(C(=O)OC(C)(C)C)CC2)cc1F]

Synthesis of tert-Butyl 4,4′-(4,4′-(4-methyl 4H-1,2,4-triazole-3,5-diyl)bis(3-fluoro-4,1-phenylene))bis(5,6-dihydropyridine-1(2H)-carboxylate: A flask was charged with 10700 mg (25.0 mmol) of 3,5-Bis(4-bromo-2-fluorophenyl)-4-methyl-4H-1,2,4-triazole, 450 mg (2.0 mmol) of palladium acetate, 2060 mg (5.0 mmol) of 2-dicyclohexylphosphino-2′,6′-dimethoxybiphenyl, 18,500 mg (51.9 mmol) of tert-butyl 4-(4,4,5,5-tetramethyl-1,3,2-dioxaborolan-2-yl)-5,6-dihydropyridine-1(2H)-carboxylate, 150 mL of dioxane and 88 mL of 2.0 molar potassium carbonate aqueous solution. The flask was swept with nitrogen for five minutes and heated at 95° C. for 18 hours. Then the reaction was complete by LCMS and the reaction was allowed to cool and diluted with 40 mL water and 100 mL of ethyl acetate added over ten minutes. Solids formed quickly and reaction stirred for two hours. Solid collected on a filter and rinsed with 30 mL ethyl acetate and air-dried for 18 hours in the hood to yield 11.2 grams (70%).

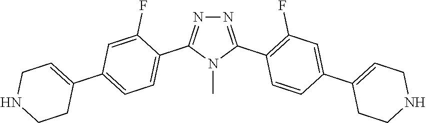 [SMILES: Cn1c(-c2ccc(C3=CCNCC3)cc2F)nnc1-c1ccc(C2=CCNCC2)cc1F]

Synthesis of 4,4′-(4,4′-(4-Methyl-4H-1,2,4-triazole-3,5-diyl)bis(3-fluoro-4,1-phenylene))bis(1,2,3,6-tetrahydropyridine): The tert-butyl 4,4′-(4,4′-(4-methyl-4H-1,2,4-triazole-3,5-diyl)bis(3-fluoro-4,1-phenylene))bis(5,6-dihydropyridine-1(2H)-carboxylate was slurried with 4500 mg (41.7 mmol) of anisole and 15 mL dichloromethane. Then 25 mL (32.7 mmol) of trifluoroacetic acid added over five minutes with stirring. 90 minutes later 1.0 mL (13.1 mmol) more trifluoroacetic acid added and one hour later the reaction was a solution. The reaction was concentrated under vacuum and triturated with two 50 mL portions of ether and ether decanted away to yield a solid in quantitative yield.

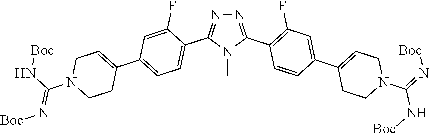 [SMILES: Cn1c(-c2ccc(C3=CCN(/C(=N\C(=O)OC(C)(C)C)NC(=O)OC(C)(C)C)CC3)cc2F)nnc1-c1ccc(C2=CCN(/C(=N/C(=O)OC(C)(C)C)NC(=O)OC(C)(C)C)CC2)cc1F]

Synthesis of tert-Butyl (4,4′-(4,4′-(4-methyl-4H-1,2,4-triazole-3,5-diyl)bis(3-fluoro-4,1-phenylene))bis(5,6-dihydropyridine-4,1(2H)-diyl))bis(methane-1-yl-1,1-diylidene)tetracarbamate: A flask was charged with 5950 mg (9.0 mmol) of 4,4′-(4,4′-(4-methyl-4H-1,2,4-triazole-3,5-diyl)bis(3-fluoro-4,1-phenylene))bis(1,2,3,6-tetrahydropyridine bis trifluoroacetic acid salt and 30 mL N,N-dimethylformamide. Then 6360 mg (63 mmol) of triethylamine added over five minutes. Then 6700 mg (39.4 mmol) of tert-butyl (1H-pyrazol-1-yl)methanediylidenedicarbamate was added over several minutes. The reaction slurry was warmed at 37° C. for five hours. The reaction was diluted with 150 mL ethyl acetate and then washed with four portions of 50 mL of water. Organic phase was dried with sodium sulfate and concentrated under vacuum. The product was purified by normal phase chromatography, loaded as dichloromethane solution, eluted with (30% acetonitrile/dichloromethane)/dichloromethane with step gradient from 0 to 100% to elute impurities. Then the expected product eluted off with (20% MeOH/dichloromethane)/dichloromethane step gradient from 0 to 100%. Yield was 5200 mg of product (63%).

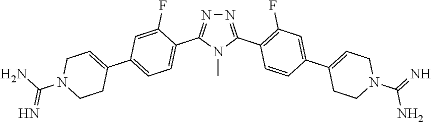 [SMILES: Cn1c(-c2ccc(C3=CCN(C(=N)N)CC3)cc2F)nnc1-c1ccc(C2=CCN(C(=N)N)CC2)cc1F]

### Example 8

Synthesis of 4,4′-(4,4′-(4-Methyl-4H-1,2,4-triazole-3,5-diyl)bis(3-fluoro-4,1-phenylene)) bis(5,6-dihydropyridine-1(2H)-carboximidamide): A flask was charged with 210 mg (0.215 mmol) of tert-butyl (4,4′-(4,4′-(4-methyl-4H-1,2,4-triazole-3,5-diyl)bis(3-fluoro-4,1-phenylene))bis(5,6-dihydropyridine-4,1(2H)-diyl))bis(methane-1-yl-1,1-diylidene)tetracarbamate was dissolved with a mixture of 500 mg (4.63 mmol) of anisole and 4 mL of dichloromethane. Then 6 mL (77.9 mmol) of trifluoroacetic acid added over two minutes with stirring and stirred for 18 hours. The next day LCMS indicted reaction complete and reaction concentrated under vacuum. Oil was triturated with two 30 mL portions of ether and ether decanted away to yield a solid. Solid was dissolved in 6 mL N,N-dimethylformamide and was purified by prep HPLC. Yield was 102 mg (64%). ¹H NMR (300 MHz, CD₃OD) δ=7.83 (d, J=8.6 Hz, 1H), 7.65-7.41 (m, 3H), 4.88-4.65 (m, 3H), 4.11 (s, 2H), 4.00 (q, J=2.8 Hz, 2H), 3.59-3.32 (m, 3H), 3.06-2.98 (m, 2H), 1.26 (s, 1H). LC/MS method A: R_{f}=2.59 mins., (M+H)⁺=518, purity >95%.

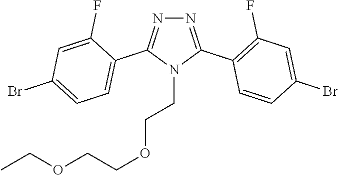 [SMILES: CCOCCOCCn1c(-c2ccc(Br)cc2F)nnc1-c1ccc(Br)cc1F]

Synthesis of 3,5-Bis(4-bromo-2-fluorophenyl)-4-(2-(2-ethoxyethoxy)ethyl)-4H-1,2,4-triazole: A vial was charged with 550 mg (1.17 mmol) of (Z)-4-bromo-N-[(1Z)-(4-bromo-2-fluorophenyl)(chloro)methylidene]-2-fluorobenzene-1-carbohydrazonoyl chloride and slurried with 5 mL of methanol. Then 187 mg (1.41 mmol) of 2-(2-ethoxyethoxy)ethanamine added over 1 minute. Five minutes later 377 mg (2.93 mmol) of N,N-diisiopropylethylamine was added over one minute. Five minutes later the reaction was heated to 65° C. and stirred for 18 hours. Then, the reaction was allowed to cool and 7 mL of water added. One hour later solids collected and rinsed with several mL of ether. The solid was dissolved with dichloromethane and chromatographed on silica gel and eluted with a step gradient from 0 to 100% ethyl acetate/dichloromethane. Yield was 160 mg (27%).

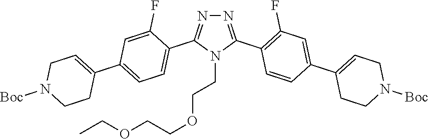 [SMILES: CCOCCOCCn1c(-c2ccc(C3=CCN(C(=O)OC(C)(C)C)CC3)cc2F)nnc1-c1ccc(C2=CCN(C(=O)OC(C)(C)C)CC2)cc1F]

Synthesis of tert-Butyl 4,4′-(4,4′-(4-(2-(2-ethoxyethoxy)ethyl)-4H-1,2,4-triazole-3,5-diyl)bis(3-fluoro-4,1-phenylene))bis(5,6-dihydropyridine-1(2H)-carboxylate): A flask was charged with 160 mg (0.30 mmol of 3,5-bis(4-bromo-2-fluorophenyl)-4-(2-(2-ethoxyethoxy)ethyl)-4H-1,2,4-triazole and 5.4 mg (0.024 mmol) of palladium acetate, 25 mg (0.06 mmol) of 2-dicyclohexylphosphino-2′,6′-dimethoxybiphenyl, 234 mg (0.75 mmol) of tert-butyl 4-(4,4,5,5-tetramethyl-1,3,2-dioxaborolan-2-yl)-5,6-dihydropyridine-1(2H)-carboxylate, 2 mL of 1,4-dioxane and 1.1 mL of 2.0 molar potassium carbonate aqueous solution. The flask was swept with nitrogen for five minutes and heated at 95° C. for 18 hours. The reaction was complete by LCMS and the reaction was allowed to cool and diluted with 20 mL water and 25 mL of ethyl acetate added over five minutes. Solids formed quickly and reaction stirred for two hours. Solid collected on a filter and rinsed with 30 mL ethyl acetate and air-dried for 18 hours in the hood to yield 239 mg of crude in quantitative yield.

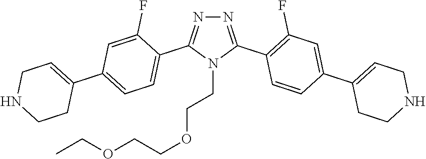 [SMILES: CCOCCOCCn1c(-c2ccc(C3=CCNCC3)cc2F)nnc1-c1ccc(C2=CCNCC2)cc1F]

Synthesis of 4,4′-(4,4′-(4-(2-(2-Ethoxyethoxy)ethyl)-4H-1,2,4-triazole-3,5-diyl)bis(3-fluoro-4,1-phenylene))bis(1,2,3,6-tetrahydropyridine: A flask was charged with 239 mg (0.30 mmol) of tert-butyl 4,4′-(4,4′-(4-(2-(2-ethoxyethoxy)ethyl)-4H-1,2,4-triazole-3,5-diyl)bis(3-fluoro-4,1-phenylene))bis(5,6-dihydropyridine-1(2H)-carboxylate) was dissolved with 500 mg (4.6 mmol) of anisole and 4 mL dichloromethane. Then 6 mL (78 mmol) of trifluoroacetic acid added over five minutes with stirring. One hour later the reaction was concentrated under vacuum and triturated with two 30 mL, portions of ether and ether decanted away to yield a solid in quantitative yield.

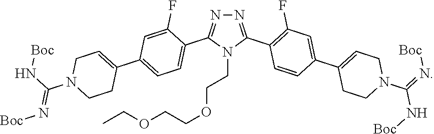 [SMILES: CCOCCOCCn1c(-c2ccc(C3=CCN(/C(=N\C(=O)OC(C)(C)C)NC(=O)OC(C)(C)C)CC3)cc2F)nnc1-c1ccc(C2=CCN(/C(=N/C(=O)OC(C)(C)C)NC(=O)OC(C)(C)C)CC2)cc1F]

Synthesis of tert-Butyl (4,4′-(4,4′-(4-(2-(2-ethoxyethoxy)ethyl)-4H-1,2,4-triazole-3,5-diyl)bis(3-fluoro-4,1-phenylene))bis(5,6-dihydropyridine-4,1(2H)-diyl))bis (methane-1-yl-1,1-diylidene)tetracarbamate: A flask was charged with 229 mg (0.3 mmol) of 4,4′-(4,4′-(4-(2-(2-ethoxyethoxy)ethyl)-4H-1,2,4-triazole-3,5-diyl)bis(3-fluoro-4,1-phenylene))bis(1,2,3,6-tetrahydropyridine bis trifluoroacetic acid salt and 4 mL N,N-dimethylformamide. Then 212 mg (2.1 mmol) of triethylamine added over one minute. Then 223 mg (0.75 mmol) of tert-butyl (1H-pyrazol-1-yl)methanediylidenedicarbamate was added at once. The reaction was stirred for five hours and 100 mg of tert-butyl (1H-pyrazol-1-yl) methanediylidenedicarbamate added. After 18 hours 140 mg (1.39 mmol) of triethylamine and 0.100 mg (0.32 mmol) of tert-butyl (1H-pyrazol-1-yl)methanediylidene dicarbamate were added at once and reaction stirred for 18 hours at room temp. After stirring for 18 hours, the reaction was diluted with 30 mL ethyl acetate and washed with three 20 mL, portions of water, dried and concentrated under vacuum. The concentrate was dissolved with dichloromethane and chromatographed on silica and eluted with an ethyl acetate/dichloromethane step gradient from 0 to 100%. Similar fractions combined and concentrated under vacuum. Yield was 187 mg (61% yield).

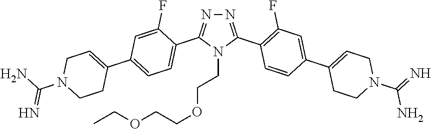 [SMILES: CCOCCOCCn1c(-c2ccc(C3=CCN(C(=N)N)CC3)cc2F)nnc1-c1ccc(C2=CCN(C(=N)N)CC2)cc1F]

### Example 9

Synthesis of 4,4′-(4,4′-(4-(2-(2-Ethoxyethoxy)ethyl)-4H-1,2,4-triazole-3,5-diyl)bis(3-fluoro-4,1-phenylene))bis(5,6-dihydropyridine-1(2H)-carboximidamide): A flask was charged with 0.180 mg (0.21 mmol) 4,4′-(4,4′-(4-(2-(2-Ethoxyethoxy)ethyl)-4H-1,2,4-triazole-3,5-diyl)bis(3-fluoro-4,1-phenylene))bis(5,6-dihydropyridine-1(2H)-carboximidamide) from tert-butyl (4,4′-(4,4′-(4-(2-(2-ethoxyethoxy)ethyl)-4H-1,2,4-triazole-3,5-diyl)bis(3-fluoro-4,1-phenylene))bis (5,6-dihydropyridine-4,1(2H)-diyl))bis(methane-1-yl-1,1-di ylidene)tetracarbamate was dissolved with a mixture of 500 mg (4.6 mmol) of anisole and 3 mL of dichloromethane. Then 6 mL (77.9 mmol) of trifluoroacetic acid added over five minutes with stirring. Two hours later LCMS indicted reaction complete and reaction concentrated under vacuum. Oil was triturated with two 30 mL portions of ether and ether decanted away to yield a solid. Solid was dried yield was 111 mg (59%). ¹H NMR (300 MHz, CD₃OD) δ=7.73 (d, J=1.1 Hz, 2H), 7.57 (s, 1H), 7.55 (s, 1H), 7.51 (s, 2H), 6.41 (s, 2H), 4.21-4.18 (m, 2H), 3.76-3.72 (m, 2H), 3.32-3.31 (m, 2H), 3.28-3.26 (m, 2H), 2.76-2.74 (m, 2H), 1.10 (br s, 3H). LC/MS method A: R_{f}=2.77 mins., (M+H)⁺=621, 311, purity >95%.

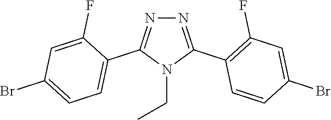 [SMILES: CCn1c(-c2ccc(Br)cc2F)nnc1-c1ccc(Br)cc1F]

Synthesis of 3,5-Bis(4-bromo-2-fluorophenyl)-4-ethyl-4H-1,2,4-triazole: A flask was charged with the crude (Z)-4-bromo-N-[(1Z)-(4-bromo-2-fluorophenyl)(chloro)methylidene]-2-fluorobenzene-1-carbohydrazonoyl chloride (500 mg, 1.13 mmol) was slurried with 15 mL of methanol and the ethylamine hydrochloride (324 mg, 3.95 mol) was added at once. Then 656 mg (5.09 mmol) of diisopropylethylamine was added over two minutes. After 30 minutes the reaction was slowly warmed to 65° C. and 18 hours and then the reaction was cooled and methanol removed under vacuum and six mL N,N-dimethylformamide added and reaction heated at 90° C. for 245 hours and then 100° C. for 24 hours. After cooling, 20 mL of water added over several minutes. One hour later the solid was collected, air dried for 15 minutes and rinsed with ten mL of ether and air dried in the hood for several hours. Second crop of solid was isolated from mother liquor, worked-up as first crop and combined. Yield was 250 mg (50%).

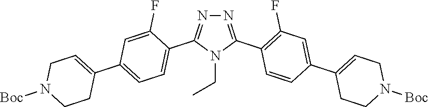 [SMILES: CCn1c(-c2ccc(C3=CCN(C(=O)OC(C)(C)C)CC3)cc2F)nnc1-c1ccc(C2=CCN(C(=O)OC(C)(C)C)CC2)cc1F]

Synthesis of tert-Butyl 4,4′-(4,4′-(4-ethyl-4H-1,2,4-triazole-3,5-diyl)bis(3-fluoro-4,1-phenylene))bis(5,6-dihydropyridine-1(2H)-carboxylate): A flask was charged with 200 mg (0.564 mmol) of 3,5-bis(4-bromo-2-fluorophenyl)-4-ethyl-4H-1,2,4-triazole, 10 mg (0.045 mmol) of palladium acetate, 46 mg (0.113 mmol) of 2-dicyclohexylphosphino-2′,6′-dimethoxybiphenyl, 437 mg (1.41 mmol) of tert-butyl 4-(4,4,5,5-tetramethyl-1,3,2-dioxaborolan-2-yl)-5,6-dihydropyridine-1(2H)-carboxylate, 4 mL of dioxane and 2.3 mL of 2.0 molar potassium carbonate aqueous solution. The flask was swept with nitrogen for five minutes and heated at 95° C. for 18 hours. The reaction was complete by LCMS and the reaction was allowed to cool and diluted with 3 mL water and 5 mL of ethyl acetate added over two minutes. No solids formed. Then 15 mL ethyl acetate and 10 mL water added. Organic phase was dried and concentrated under vacuum to yield 300 mg (82%).

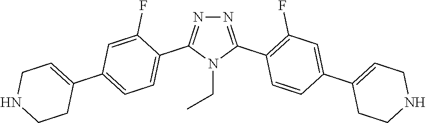 [SMILES: CCn1c(-c2ccc(C3=CCNCC3)cc2F)nnc1-c1ccc(C2=CCNCC2)cc1F]

Synthesis of 4,4′-(4,4′-(4-Ethyl-4H-1,2,4-triazole-3,5-di)1)bis(3-fluoro-4,1-phenylene))bis(1,2,3,6-tetrahydropyridine): A flask was charged with 300 mg (0.464 mmol) of tert-butyl 4,4′-(4,4′-(4-ethyl-4H-1,2,4-triazole-3,5-diyl)bis(3-fluoro-4,1-phenylene))bis (5,6-dihydropyridine-1(2H)-carboxylate was slurried with 500 mg (4.63 mmol) of anisole and 4 mL dichloromethane. Then 10 mL (130 mmol) of trifluoroacetic acid added over five minutes with stirring. One hour later the reaction was concentrated under vacuum and triturated with two 30 ml, portions of ether and ether decanted away to yield a solid in quantitative yield.

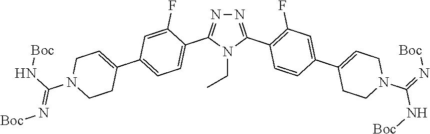 [SMILES: CCn1c(-c2ccc(C3=CCN(/C(=N\C(=O)OC(C)(C)C)NC(=O)OC(C)(C)C)CC3)cc2F)nnc1-c1ccc(C2=CCN(/C(=N/C(=O)OC(C)(C)C)NC(=O)OC(C)(C)C)CC2)cc1F]

Synthesis of tert-Butyl (4,4′-(4,4′-(4-ethyl-4H-1,2,4-triazole-3,5-diyl)bis(3-fluoro-4,1-phenylene))bis(5,6-dihydropyridine-4,1(2H)-diyl))bis(methane-1-yl-1,1-diylidene) tetracarbamate: A flask was charged with 313 mg (0.464 mmol) of 4,4′-(4,4′-(4-ethyl-4H-1,2,4-triazole-3,5-diyl)bis(3-fluoro-4,1-phenylene))bis(1,2,3,6-tetrahydropyridine bis trifluoroacetic acid salt and 3 mL N,N-dimethylformamide. Then 396 mg (3.92 mmol) of triethylamine added over two minutes. Then 521 mg (1.68 mmol) of tert-butyl. (1H-pyrazol-1-yl)methanediylidenedicarbamate was added at once and stirred for 18 hours at 23° C. The reaction was diluted with 30 mL ethyl acetate and then washed with three portions of 20 mL of water. Organic phase was dried with sodium sulfate and concentrated under vacuum. The product was purified by normal phase chromatography, loaded as dichloromethane solution, eluted with (30% acetonitrile/dichloromethane)/dichloromethane with step gradient from 0 to 100% to elute impurities. Then the expected product eluted off with (20% methanol/dichloromethane)/dichloromethane step gradient from 0 to 100%. Yield was 300 mg of product (69%).

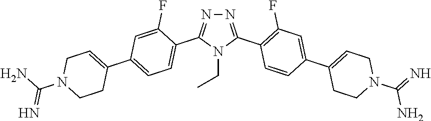 [SMILES: CCn1c(-c2ccc(C3=CCN(C(=N)N)CC3)cc2F)nnc1-c1ccc(C2=CCN(C(=N)N)CC2)cc1F]

### Example 10

Synthesis of 4,4′-(4,4′-(4-Ethyl-4H-1,2,4-triazole-3,5-diyl)bis(3-fluoro-4,1-phenylene)) bis(5,6-dihydropyridine-1(2H)-carboximidamide): A flask was charged with 200 mg (0.215 mmol) of tert-butyl (4,4′-(4,4′-(4-ethyl-4H-1,2,4-triazole-3,5-diyl)bis(3-fluoro-4,1-phenylene)) bis(5,6-dihydropyridine-4,1(2H)-diyl))bis(methane-1-yl-1,1-diylidene)tetracarbamate was dissolved with a mixture of 500 mg (4.63 mmol) of anisole and 3 mL of dichloromethane. Then 6 mL (77.9 mmol) of trifluoroacetic acid added over two minutes with stirring and reaction stirred for 18 hours. After four hours the reaction was concentrated under vacuum. Oil was triturated with two 20 mL portions of ether and ether decanted away to yield an oil. Solid was dissolved in 20% acetonitrile/water and was purified by prep HPLC. Yield was 99 mg (60%). ¹H NMR (300 MHz, CD₃OD) δ=7.66 (t, 0.7.6 Hz, 3H), 7.55 (d, J=8.5 Hz, 3H), 6.40 (s, 2H), 4.19 (br d, J=3.2 Hz, 4H), 3.74 (t, J=5.7 Hz, 4H), 2.15 (br. s, 4H), 1.29 (br s, 3H). LC/MS method A: R_{f}=2.67 mins., (M+H)⁺=532, purity >95%.

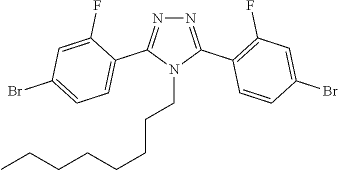 [SMILES: CCCCCCCCn1c(-c2ccc(Br)cc2F)nnc1-c1ccc(Br)cc1F]

Synthesis of 3,5-bis(4-Bromo-2-fluorophenyl)-4-octyl-4H-1,2,4-triazole: The crude (Z)-4-bromo-N-[(1Z)-(4-bromo-2-fluorophenyl)(chloro)methylidene]-2-fluorobenzene-1-carbohydrazonoyl chloride (590 mg, 1.25 mmol) was slurried with 5 mL of methanol and octylamine hydrochloride (2.38 mg, 1.50 mol) was added at once. Then 240 mg (1.88 mmol) of N,N-diisopropylethylamine was added over one minute. After 30 minutes the reaction was slowly warmed to 65° C. for 30 minutes and reaction cooled and 350 mg (2.71 mmol) of N,N-diisopropylethylamine added at once. The reaction was heated at 65° C. for 72 hours. The reaction was cooled and most of methanol removed under vacuum. Then the concentrate was partitioned between 20 mL ethyl acetate and 20 mL of water. The organic phase was washed with 30 mL 10% sodium hydrogen sulfate and 20 mL of water and concentrated under vacuum. The concentrate was chromatographed on silica and eluted with an ethyl acetate/dichloromethane step gradient from 0 to 0.100% Yield=220 mg (33%).

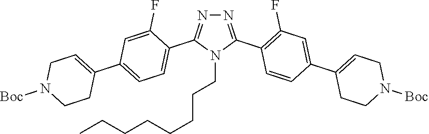 [SMILES: CCCCCCCCn1c(-c2ccc(C3=CCN(C(=O)OC(C)(C)C)CC3)cc2F)nnc1-c1ccc(C2=CCN(C(=O)OC(C)(C)C)CC2)cc1F]

Synthesis of tert-Butyl 4,4′-(4,4′-(4-octyl-4H-1,2,4-triazole-3,5-diyl)bis(3-fluoro-4,1-phenylene))bis(5,6-dihydropyridine-1(2H)-carboxylate): A flask was charged with 220 mg (0.417 mmol) of 3,5-bis(4-bromo-2-fluorophenyl)-4-octyl-4H-1,2,4-triazole, 7.5 mg (0.033 mmol) of palladium acetate, 34 mg (0.83 mmol) of S-Phos, 382 mg (1.04 mmol) of tert-butyl 4-(4,4,5,5-tetramethyl-1,3,2-dioxaborolan-2-yl)-5,6-dihydropyridine-1(2H)-carboxylate, 4 mL of dioxane and 1.6 mL of 2.0 molar potassium carbonate aqueous solution. The flask was swept with nitrogen for five minutes and heated at 95° C. for 18 hours. The reaction was complete by LCMS and the reaction was allowed to cool and diluted with 20 mL water and 30 mL of ethyl acetate. The organic phase was dried and concentrated under vacuum. The concentrate was chromatographed on silica with an ethyl acetate/dichloromethane step gradient from 0 to 100%. Similar fractions combined and concentrated under vacuum to yield air-dried for 18 hours in the hood to yield 230 mg (75%).

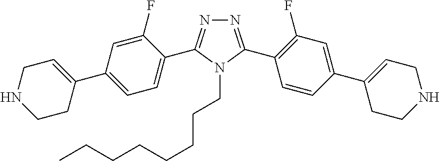 [SMILES: CCCCCCCCn1c(-c2ccc(C3=CCNCC3)cc2F)nnc1-c1ccc(C2=CCNCC2)cc1F]

Synthesis of 4,4′-(4,4′-(4-Octyl-4H-1,2,4-triazole-3,5-diyl)bis(3-fluoro-4,1-phenylene)) bis(1,2,3,6-tetrahydropyridine): A flask was charged with 230 mg (0.31 mmol) of tert-butyl 4,4′-(4,4′-(4-octyl-4H-1,2,4-triazole-3,5-diyl)bis(3-fluoro-4,1-phenylene)) bis(5,6-dihydropyridine-1(2H)-carboxylate was slurried with 500 mg (4.6 mmol) of anisole and 3 mL dichloromethane. Then 6 mL (77.9 mmol) of trifluoroacetic acid added over two minutes with stirring. One hour later the reaction was concentrated under vacuum and triturated with two 20 mL portions of ether and ether decanted away to yield a solid in quantitative yield.

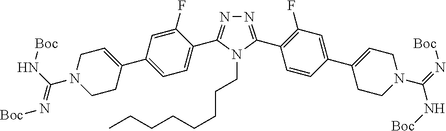 [SMILES: CCCCCCCCn1c(-c2ccc(C3=CCN(/C(=N\C(=O)OC(C)(C)C)NC(=O)OC(C)(C)C)CC3)cc2F)nnc1-c1ccc(C2=CCN(/C(=N/C(=O)OC(C)(C)C)NC(=O)OC(C)(C)C)CC2)cc1F]

Synthesis of tert-Butyl (4,4′-(4,4′-(4-octyl-4H-1,2,4-triazole-3,5-diyl)bis(3-fluoro-4,1-phenylene))bis(5,6-dihydropyridine-4,1(2H)-diyl))bis(methane-1-yl-1,1-diylidene) tetracarbamate: A flask was charged with 290 mg (0.30 mmol) of 4,4′-(4,4′-(4-octyl-4H-1,2,4-triazole-3,5-diyl)bis(3-fluoro-4,1-phenylene))bis(1,2,3,6-tetrahydropyridine bis trifluoroacetic acid salt and 3 mL N,N-dimethylformamide. Then 212 mg (2.1 mmol) of triethylamine added over one minute. Then 232 mg (0.75 mmol) of tert-butyl (1H-pyrazol-1-yl)methanediylidenedicarbamate was added at once. The reaction was stirred at 23° C. for 18 hours. The reaction was diluted with 30 mL ethyl acetate and then washed with three portions of 20 mL of water. Organic phase was dried with sodium sulfate and concentrated under vacuum. The concentrate was dissolved with dichloromethane and purified by silica chromatography, eluted with ethyl acetate/dichloromethane with step gradient from 0 to 100%. Similar fractions were combined and concentrated under vacuum to yield 170 mg (57% yield) of product.

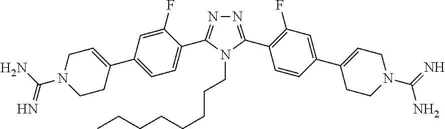 [SMILES: CCCCCCCCn1c(-c2ccc(C3=CCN(C(=N)N)CC3)cc2F)nnc1-c1ccc(C2=CCN(C(=N)N)CC2)cc1F]

### Example 11

Synthesis of 4,4′-(4,4′-(4-Octyl-4H-1,2,4-triazole-3,5-diyl)bis(3-fluoro-4,1-phenylene))bis(5,6-dihydropyridine-1(2H)-carboximidamide): A flask was charged with 170 mg (0.16 mmol) of tert-butyl. (4,4′-(4,4′-(4-octyl-4H-1,2,4-triazole-3,5-diyl)bis(3-fluoro-4,1-phenylene)) bis(5,6-dihydropyridine-4,1(2H)-diyl))bis(methane-1-yl-1,1-diylidene)tetracarbamate was dissolved with 3 mL of dichloromethane. Then 5 mL (64.9 mmol) of trifluoroacetic acid was added over three minutes with stirring. Three hours later the reaction was concentrated under vacuum The concentrate was triturated with two 20 mL portions of ether and ether decanted away to yield 80 mg (58% yield) of product. ¹H NMR (300 MHz, methanol-d₄) δ=7.69-7.64 (m, 2H), 7.58-7.54 (m, 2H), 7.51 (d, J=1.6 Hz, 2H), 6.43-6.40 (m, 2H), 4.21-4.17 (m, 2H), 4.01 (t, J=7.1 Hz, 2H), 3.74 (t, J=5.6 Hz, 4H), 3.32-3.31 (m, 4H), 2.75 (br dd, J=2.3, 3.7 Hz, 8H), 1.38-1.32 (m, 2H), 1.17 (br t, J=7.1 Hz, 2H), 1.09-0.91 (m, 2H), 0.85-0.79 (m, 3H). LC/MS method A: R_{f}=3.37 mins., (M+H)⁺=617,309, purity >95%.

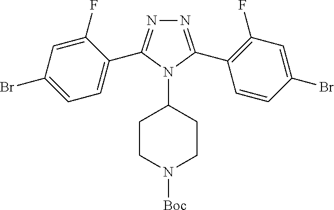 [SMILES: CC(C)(C)OC(=O)N1CCC(n2c(-c3ccc(Br)cc3F)nnc2-c2ccc(Br)cc2F)CC1]

Synthesis of tert-Butyl 4-(3,5-bis(4-bromo-2-fluorophenyl)-4H-1,2,4-triazol-4-yl)piperidine-1-carboxylate): A flask was charged with crude (Z)-4-bromo-N-[(1Z)-(4-bromo-2-fluorophenyl)(chloro)methylidene]-2-fluorobenzene-1-carbohydrazonoyl chloride (1.42 grams, 3.0 mmol) was slurried with 15 mL of methanol and the tert-butyl 4-aminopiperidine-1-carboxylate (1330 mg, 6.63 mol) was added at once. Then 1550 mg (12.0 mmol) of diisopropylethylamine was added over two minutes and reaction warmed to 60° C. for 18 hours. The reaction was heated to 75° C. for an additional 18 hours. The reaction was allowed to cool and then 50 mL of water added over several minutes. The solid was removed by filtration and aqueous phase extracted with 50 mL, ethyl acetate. Organic phase was dried and concentrated under vacuum. The concentrate was dissolved with 8 mL of N,N-dimethylformamide and 2 mL N,N-diisopropylethylamine and heated to 95° C. for 0.18 hours and then 0.105° C. for 18 hours. Then reaction was then heated to 120° C. for an additional 18 hours and allowed to cool and 1 mL glacial acetic acid added, and heated at 110° C. for 18 hours, heated at 120° C. for 18 hours and at 140° C. for two hours and allowed to cool to 23° C. The reaction was diluted with 60 mL ethyl acetate and washed with 20 mL water, 40 mL of 10% sodium hydrogen sulfate and two 20 mL portions of saturated brine, dried and concentrated under vacuum. The concentrate was chromatographed on silica and eluted with ethyl acetate/dichloromethane step gradient from 0 to 100%. Similar fractions combined and concentrated under vacuum to yield 240 mg (13%) of product.

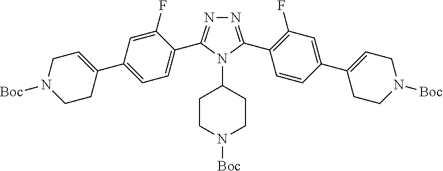 [SMILES: CC(C)(C)OC(=O)N1CC=C(c2ccc(-c3nnc(-c4ccc(C5=CCN(C(=O)OC(C)(C)C)CC5)cc4F)n3C3CCN(C(=O)OC(C)(C)C)CC3)c(F)c2)CC1]

Synthesis of tert-Butyl 4,4′-(4,4′-(4-(1-(tert-butoxycarbonyl)piperidin-4-yl)-4H-1,2,4-triazole-3,5-diyl)bis(3-fluoro-4,1-phenylene))bis(5,6-dihydropyridine-1(2H)-carboxylate): A flask was charged with 240 mg (0.40 mmol) of tert-butyl 4-(3,5-bis(4-bromo-2-fluorophenyl)-4H-1,2,4-triazol-4-yl)piperidine-1-carboxylate), 7.2 mg (0.032 mmol) of palladium acetate, 33 mg (0.08 mmol) of 2-dicyclohexylphosphino-2′,6′-dimethoxybiphenyl, 309 mg (1.0 mmol) of tert-butyl 4-(4,4,5,5-tetramethyl-1,3,2-dioxaborolan-2-yl)-5,6-dihydropyridine-1(2H)-carboxylate, 4 mL of 1,4-dioxane and 1.4 of 2.0 molar potassium carbonate aqueous solution. The flask was swept with nitrogen for five minutes and heated at 95° C. for 18 hours. The reaction was complete by LCMS and the reaction was allowed to cool and diluted with 20 mL water and 30 mL of ethyl acetate added over five minutes. The organic phase was dried and concentrated under vacuum to yield the product in quantitative yield.

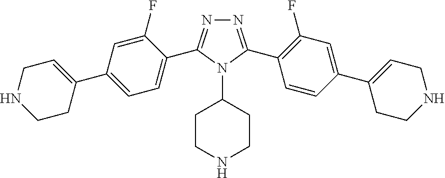 [SMILES: Fc1cc(C2=CCNCC2)ccc1-c1nnc(-c2ccc(C3=CCNCC3)cc2F)n1C1CCNCC1]

Synthesis of 4,4′-(4,4′-(4-(Piperidin-4-yl)-4H-1,2,4-triazole-3,5-diyl)bis(3-fluoro-4,1-phenylene))bis(1,2,3,6-tetrahydropyridine): A flask was charged with 320 mg (0.40 mmol) of tert-butyl 4,4-(4,4-(4-(1-(tert-butoxycarbonyl)piperidin-4-yl)-4H-1,2,4-triazole-3,5-diyl)bis(3-fluoro-4,1-phenylene))bis(5,6-dihydropyridine-1(2H)-carboxylate). Then 3 mL dichloromethane and 500 mg (4.63 mmol) of anisole added. Then 6 mL (77.9 mmol) of trifluoracetic acid added dropwise over five minutes with stirring. Two hours later the reaction was concentrated under vacuum and triturated with two 50 mL portions of ether and ether decanted away to yield a solid in quantitative yield which was used without further purification.

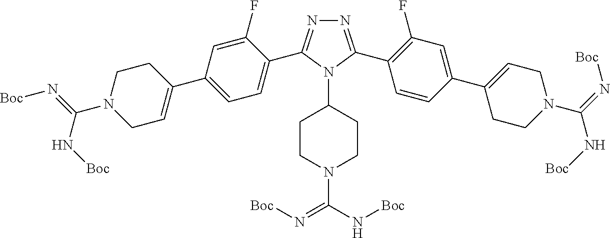 [SMILES: CC(C)(C)OC(=O)/N=C(/NC(=O)OC(C)(C)C)N1CC=C(c2ccc(-c3nnc(-c4ccc(C5=CCN(/C(=N/C(=O)OC(C)(C)C)NC(=O)OC(C)(C)C)CC5)cc4F)n3C3CCN(/C(=N\C(=O)OC(C)(C)C)NC(=O)OC(C)(C)C)CC3)c(F)c2)CC1]

Synthesis of tert-Butyl N—[(Z)-(4-{4-[5-(4-{1-[(E)-{[(tert-butoxy)-carbonyl]amino}({[(tert-butoxy)carbonyl]imino})methyl]-1,2,3,6-tetrahydropyridin-4-yl}-2-fluoro phenyl)-4-(1-methylpiperidin-4-yl)-4H-1,2,4-triazol-3-yl]-3-fluorophenyl}-1,2,3,6-tetrahydro pyridin-1-yl)({[(tert-butoxy)carbonyl]imino})methyl]carbamate: A flask was charged with 245 mg (0.35 mmol) of 4,4′-(4,4′-(4-methyl-4H-1,2,4-triazole-3,5-diyl)bis(3-fluoro-4,1-phenylene))bis(1,2,3,6-tetrahydropyridine bis trifluoroacetic acid salt and 4 mL N,N-dimethylformamide. Then 283 mg (2.80 mmol) of triethylamine added over two minutes. Then 390 mg (1.26 mmol) of tert-butyl (1H-pyrazol-1-yl)methanediylidenedicarbamate was added at once. The reaction slurry was stirred at 23° C. for 18 hours. The reaction was diluted with 30 mL of ethyl acetate and then washed with three portions of 20 mL of water. Organic phase was dried with sodium sulfate and concentrated under vacuum. The product was purified by normal phase chromatography, loaded as dichloromethane solution, eluted with ethyl acetate/dichloromethane step gradient from 0 to 100%. Yield=220 mg of product (49%).

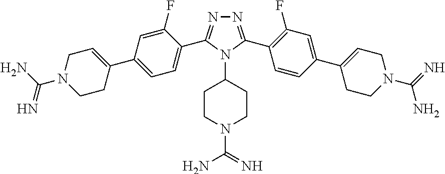 [SMILES: N=C(N)N1CC=C(c2ccc(-c3nnc(-c4ccc(C5=CCN(C(=N)N)CC5)cc4F)n3C3CCN(C(=N)N)CC3)c(F)c2)CC1]

### Example 12

Synthesis of 4,4′-(4,4′-(4-(1-Carbamimidoylpiperidin-4-yl)-4H-1,2,4-triazole-3,5-diyl)bis (3-fluoro-4,1-phenylene))bis(5,6-dihydropyridine-1(2H)-carboximidamide): A flask was charged with 210 mg (0.17 mmol) of tert-butyl N—[(Z)-(4 {4-[5-(4-{1-[(E)-{[(tert-butoxy)-carbonyl]amino}({[(tert-butoxy)carbonyl]imino})methyl]-1,2,3,6-tetrahydropyridin-4-yl}-2-fluoro phenyl)-4-(1-methylpiperidin-4-yl)-4H-1,2,4-triazol-3-yl]-3-fluorophenyl}-1,2,3,6-tetrahydropyridin-1-yl)({[(tert-butoxy)carbonyl]imino})methyl] carbamate was dissolved with a mixture of 500 mg (4.6 mmol) of anisole and 3 mL of dichloromethane. Then 5 mL (65 mmol) of trifluoroacetic acid added over five minutes with stirring. Two hours later the reaction was concentrated under vacuum. Oil was triturated with two 30 mL portions of ether and ether decanted away to yield a solid. Solid was dissolved in N,N-dimethylformamide and was purified by prep HPLC. Yield was 48 mg (14%). ¹H NMR (300 MHz, methanol-d₄) δ=7.68-7.65 (m, 1H), 7.63 (s, 1H), 7.57-7.53 (m, 3H), 7.50 (d, J=1.6 Hz, 1H), 6.42-6.39 (m, 2H), 4.21-4.18 (m, 3H), 3.83 (br s, 2H), 3.74 (t, J=5.6 Hz, 4H), 3.01 (br s, 2H), 2.75 (br t, J=5.2 Hz, 4H), 2.10 (br d, J=9.9 Hz, 2H), 1.83-1.65 (m, 4H). LC/MS method A: R_{f}=2.28 mins., (M+H)⁺=630, 315, purity >95°

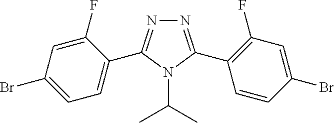 [SMILES: CC(C)n1c(-c2ccc(Br)cc2F)nnc1-c1ccc(Br)cc1F]

3,5-Bis(4-Bromo-2-fluorophenyl)-4-isopropyl-4H-1,2,4-triazole: A flask was charged with the crude (Z)-4-bromo-N-[(1Z)-(4-bromo-2-fluorophenyl)(chloro)methylidene]-2-fluorobenzene-1-carbohydrazonoyl chloride (890 mg, 1.89 mmol) and was slurried with 4 mL of methanol and the isopropylamine (368 mg, 6.23 mol) was added over five minutes. Then 175 mg (1.36 mmol) of diisopropylethylamine was added over two minutes. After 30 minutes the reaction was slowly warmed to 60° C. for 18 hours and reaction cooled and 3.0 grams (50 mmol) of glacial acetic acid was added over five minutes. The reaction was heated at 65° C. for 18 hours. The reaction was allowed to cool and most of methanol removed under vacuum. Then 40 mL of dichloromethane added and washed with three 20 mL portions of water. The organic phase was dried and concentrated under vacuum to yield 720 mg of solid product (83%).

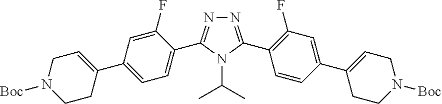 [SMILES: CC(C)n1c(-c2ccc(C3=CCN(C(=O)OC(C)(C)C)CC3)cc2F)nnc1-c1ccc(C2=CCN(C(=O)OC(C)(C)C)CC2)cc1F]

Synthesis of tert-Butyl 4,4′-(4,4′-(4-isopropyl-4H-1,2,4-triazole-3,5-diyl)bis(3-fluoro-4,1-phenylene))bis(5,6-dihydropyridine-1(2H)-carboxylate): A flask was charged with 700 mg (1.53 mmol) of 3,5-Bis(4-bromo-2-fluorophenyl)-4-isopropyl-4H-1,2,4-triazole, 28 mg (0.122 mmol) of palladium acetate, 125 mg (0.306 mmol) of 2-dicyclohexylphosphino-2′,6′-dimethoxybiphenyl, 1200 mg (3.82 mmol) of tert-butyl 4 (4,4,5,5-tetramethyl-1,3,2-dioxaborolan-2-yl)-5,6-dihydropyridine-1(2H)-carboxylate, 12 mL of dioxane and 5.8 mL of 2.0 molar potassium carbonate aqueous solution. The flask was swept with nitrogen for five minutes and heated at 95° C. for 18 hours. The reaction was complete by LCMS and the reaction was allowed to cool and diluted with 10 mL water and 25 mL of ethyl acetate. The organic phase was dried and concentrated under vacuum to yield 800 mg (79%) of product.

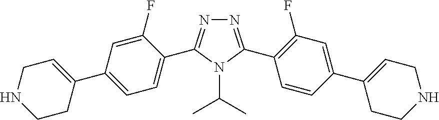 [SMILES: CC(C)n1c(-c2ccc(C3=CCNCC3)cc2F)nnc1-c1ccc(C2=CCNCC2)cc1F]

Synthesis of 4,4′-(4,4′-(4-isopropyl-4H-1,2,4-triazole-3,5-diyl)bis(3-fluoro-4,1-phenylene))bis(1,2,3,6-tetrahydropyridine: A flask was charged with 800 mg (1.21 mmol) of tert-butyl 4,4′-(4,4′-(4-isopropyl-4H-1,2,4-triazole-3,5-diyl)bis(3-fluoro-4,1-phenylene)) bis(5,6-dihydropyridine-1(2H)-carboxylate was slurried with 500 mg (4.63 mmol) of anisole and 4 mL dichloromethane. Then 6 mL (77.9 mmol) of trifluoroacetic acid was added over two minutes with stirring. Three hours later the reaction was concentrated under vacuum and triturated with two 50 mL portions of ether and ether decanted away to yield a solid in quantitative yield.

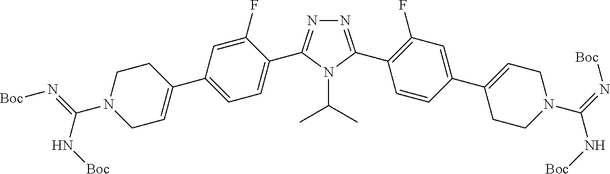 [SMILES: CC(C)n1c(-c2ccc(C3=CCN(/C(=N\C(=O)OC(C)(C)C)NC(=O)OC(C)(C)C)CC3)cc2F)nnc1-c1ccc(C2=CCN(/C(=N/C(=O)OC(C)(C)C)NC(=O)OC(C)(C)C)CC2)cc1F]

Synthesis of tert-Butyl (4,4′-(4,4′-(4-isopropyl-4H-1,2,4-triazole-3,5-diyl)bis(3-fluoro-4,1-phenylene))bis(5,6-dihydropyridine-4,1(2H)-diyl))bis(methane-1-yl-1,1-diylidene) tetracarbamate: A flask was charged with 690 mg (1.0 mmol) of 4,4′-(4,4′-(4-isopropyl-4H-1,2,4-triazole-3,5-diyl)bis(3-fluoro-4,1-phenylene))bis(1,2,3,6-tetrahydro pyridine bis trifluoroacetic acid salt and 5 mL N,N-dimethylformamide. Then 707 mg (7.0 mmol) of triethylamine added over three minutes. Then 775 mg (2.50 mmol) of tert-butyl (1H-pyrazol-1-yl)methanediylidenedicarbamate was added at once. The reaction was stirred at 23° C. for 18 hours. The reaction was diluted with 30 mL, ethyl acetate and then washed with three portions of 30 mL of water. Organic phase was dried with sodium sulfate and concentrated under vacuum. The product was purified by normal phase chromatography, loaded as dichloromethane solution, eluted with ethyl acetate/dichloromethane with a step gradient from 0 to 100% to elute product. Yield was 210 mg of product (22%).

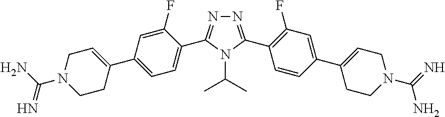 [SMILES: CC(C)n1c(-c2ccc(C3=CCN(C(=N)N)CC3)cc2F)nnc1-c1ccc(C2=CCN(C(=N)N)CC2)cc1F]

### Example 13

Synthesis of 4,4-(4,4-(4-Isopropyl-4H-1,2,4-triazole-3,5-diyl)bis(3-fluoro-4,1-phenylene))bis(5,6-dihydropyridine-1(2H)-carboximidamide): A flask was charged with 210 mg (0.22 mmol) of tert-Butyl (4,4′-(4,4′-(4-isopropyl-4H-1,2,4-triazole-3,5-diyl)bis(3-fluoro-4,1-phenylene))bis(5,6-dihydropyridine-4,1(2H)-diyl))bis(methane-1-yl-1,1-diylidene)tetracarbamate was dissolved with a mixture of 500 mg (4.6 mmol) of anisole and 4 mL of dichloromethane. Then 6 mL (7.8 mmol) of trifluoroacetic acid added over two minutes with stirring. After stirring for 18 hours at 23° C. LCMS indicated reaction complete and reaction concentrated under vacuum. Oil was triturated with two 50 mL portions of ether and ether decanted away to yield a solid. Solid was dissolved in 6 mL N,N-dimethylformamide and was purified by prep HPLC. Yield was 156 mg (92%). ¹H NMR (300 MHz, CD₃OD) δ=7.68-7.65 (m, 1H), 7.63 (s, 1H), 7.57-7.53 (m, 3H), 7.50 (d, J=1.6 Hz, 1H), 6.42-6.39 (m, 2H), 4.21-4.18 (m, 3H), 3.83 (br s, 2H), 3.74 (t, J=5.6 Hz, 4H), 3.01 (br br s, 2H), 2.75 (br t, J=5.2 Hz, 4H), 2.10 (br d, J=9.9 Hz, 2H), 1.83-1.65 (m, 4H). LC/MS method A: R_{f}=2.75 mins., (M+H)⁺=547, purity >95%.

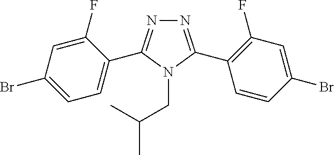 [SMILES: CC(C)Cn1c(-c2ccc(Br)cc2F)nnc1-c1ccc(Br)cc1F]

Synthesis of 3,5-bis(4-Bromo-2-fluorophenyl)-4-isobutyl-4H-1,2,4-triazole: A flask was charged with the crude (Z)-4-bromo-N-[(1Z)-(4-bromo-2-fluorophenyl)(chloro)methylidene]-2-fluorobenzene-1-carbohydrazonoyl chloride (1070 mg, 2.27 mmol) was slurried with 4 mL of methanol and the isobutylamine (208 mg, 2.84 mol) was added over two minutes. Then 732 mg (5.68 mmol) of diisopropylethylamine was added over two minutes. After ten minutes the reaction was slowly warmed to 65° C. for three days and 70° C. for 18 hours. The reaction cooled and 5 mL water added over one minute. Solids quickly formed and collected after one hour washed with 5 mL water and several mL of ether. Product soluble in ether phase and solid combined and concentrated under vacuum to yield 710 mg of solid product. (66%)

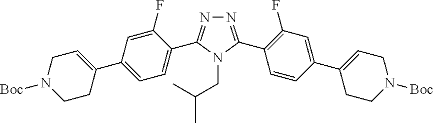 [SMILES: CC(C)Cn1c(-c2ccc(C3=CCN(C(=O)OC(C)(C)C)CC3)cc2F)nnc1-c1ccc(C2=CCN(C(=O)OC(C)(C)C)CC2)cc1F]

Synthesis of tert-Butyl 4,4′-(4,4′-(4-isobutyl-4H-1,2,4-triazole-3,5-diyl)bis(3-fluoro-4,1-phenylene))bis(5,6-dihydropyridine-1(2H)-carboxylate): A flask was charged with 700 mg (1.49 mmol) of 3,5-Bis(4-bromo-2-fluorophenyl)-4-isobutyl-4H-1,2,4-triazole, 27 mg (0.12 mmol) of palladium acetate, 122 mg (0.30 mmol) of 2-dicyclohexylphosphino-2′,6′-dimethoxybiphenyl, 1150 mg (3.72 mmol) of tert-butyl 4-(4,4,5,5-tetramethyl-1,3,2-dioxaborolan-2-yl)-5,6-dihydropyridine-1(2H)-carboxylate, 11 mL of 1,4-dioxane and 5.5 mL of 2.0 molar potassium carbonate aqueous solution. The flask was swept with nitrogen for five minutes and heated at 95° C. for 18 hours. The reaction was complete by LCMS and the reaction was allowed to cool and diluted with 15 mL water and 10 mL, of ethyl acetate added over two minutes. Solids formed slowly and reaction stirred for one hour. Solid collected on a filter and rinsed with 5 mL of water. Ethyl acetate/dioxane phase also contained product and combined with solid and concentrated under vacuum and weighed 700 mg after concentrating under vacuum. (68%).

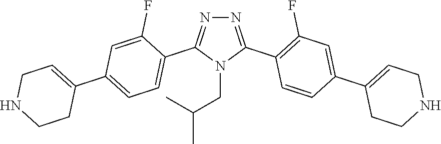 [SMILES: CC(C)Cn1c(-c2ccc(C3=CCNCC3)cc2F)nnc1-c1ccc(C2=CCNCC2)cc1F]

Synthesis of 4,4′-(4,4′-(4-isobutyl-4H-1,2,4-triazole-3,5-diyl)bis(3-fluoro-4,1-phenylene)) bis(1,2,3,6-tetrahydropyridine): A flask was charged with 700 mg (1.94 mmol) of the tert-butyl 4,4-(4,4-(4-isobutyl-4H-1,2,4-triazole-3,5-diyl)bis(3-fluoro-4,1-phenylene))bis(5,6-dihydropyridine-1(2H)-carboxylate was slurried with 500 mg (4.63 mmol) of anisole and 4 mL dichloromethane. Then 6 mL (77.9 mmol) of trifluoroacetic acid added over two minutes with stirring. Two hours later the reaction was concentrated under vacuum and triturated with two 30 mL portions of ether and ether decanted away to yield a solid in quantitative yield.

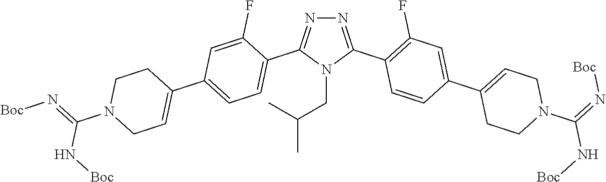 [SMILES: CC(C)Cn1c(-c2ccc(C3=CCN(/C(=N\C(=O)OC(C)(C)C)NC(=O)OC(C)(C)C)CC3)cc2F)nnc1-c1ccc(C2=CCN(/C(=N/C(=O)OC(C)(C)C)NC(=O)OC(C)(C)C)CC2)cc1F]

Synthesis of tert-Butyl (4,4′-(4,4′-(4-isobutyl-4H-1,2,4-triazole-3,5-diyl)bis(3-fluoro-4,1-phenylene))bis(5,6-dihydropyridine-4,1(2H)-diyl))bis(methane-1-yl-1,1-diylidene) tetracarbamate: A flask was charged with 630 mg (0.9 mmol) of 4,4′-(4,4′-(4-isobutyl-4H-1,2,4-triazole-3,5-diyl)bis(3-fluoro-4,1-phenylene))bis(1,2,3,6-tetrahydropyridine bis trifluoroacetic acid salt and 5 mL N,N-dimethylformamide. Then 636 mg (6.03 mmol) of triethylamine added over two minutes. Then 700 mg (2.25 mmol) of tert-butyl (1H-pyrazol-1-yl)methanediylidenedicarbamate was added over several minutes. The reaction was stirred for 18 hours at room temperature. Then the reaction was diluted with 30 mL of ethyl acetate and then washed with two portions of 30 mL of water. Organic phase was dried with sodium sulfate and concentrated under vacuum. The product was purified by normal phase chromatography, loaded as dichloromethane solution, eluted with ethyl acetate/dichloromethane with step gradient from 0 to 100% to elute product. Similar fractions combined and concentrated under vacuum. Yield was 210 mg of product (22%).

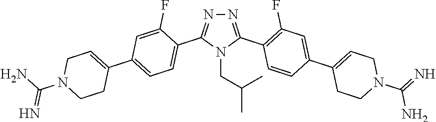 [SMILES: CC(C)Cn1c(-c2ccc(C3=CCN(C(=N)N)CC3)cc2F)nnc1-c1ccc(C2=CCN(C(=N)N)CC2)cc1F]

### Example 14

Synthesis of 4,4′-(4,4′-(4-isobutyl-4H-1,2,4-triazole-3,5-diyl)bis(3-fluoro-4,1-phenylene)) bis(5,6-dihydropyridine-1(2H)-carboximidamide)): A flask was charged with 210 mg (0.215 mmol) of tert-butyl (4,4′-(4,4′-(4-isobutyl-4H-1,2,4-triazole-3,5-diyl)bis(3-fluoro-4,1-phenylene))bis(5,6-dihydropyridine-4,1(2H)-diyl))bis(methane-1-yl-1,1-diylidene)tetracarbamate which was dissolved with a mixture of 500 mg (4.63 mmol) of anisole and 4 mL, of dichloromethane. Then 6 mL, (77.9 mmol) of trifluoroacetic acid added over two minutes with stirring and stirred for 18 hours. The next day LCMS indicted reaction was complete, and reaction concentrated under vacuum. Oil was triturated with two 30 mL portions of ether and ether decanted away to yield a solid. Solid was dissolved in 6 mL N,N-dimethylformamide and was purified by prep HPLC. Yield was 156 mg (94%). ¹H NMR (300 MHz, CD₃OD) δ=7.78-7.61 (m, 2H), 7.59-7.50 (m, 2H), 7.36 (s, 2H), 6.44-6.38 (m, 2H), 4.22-4.16 (m, 2H), 3.85 (d, J=7.7 Hz, 2H), 3.74 (t, J=5.6 Hz, 4H), 3.32-3.31 (m, 4H), 2.79-2.70 (m, 2H), 1.52 (m, 2H), 0.55 (d, J=6.7 Hz, 6H). LC/MS method A: R_{f}=2.87 mins., (M+H)⁺=561, purity >95%.

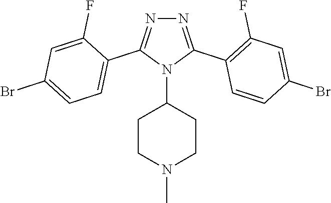 [SMILES: CN1CCC(n2c(-c3ccc(Br)cc3F)nnc2-c2ccc(Br)cc2F)CC1]

Synthesis of 4-(3,5-bis(4-Bromo-2-fluorophenyl)-4H-1,2,4-triazol-4-yl)-1-methylpiperidine: A flask was charged with the crude (Z)-4-bromo-N-[(1Z)-(4-bromo-2-fluorophenyl)(chloro)methylidene]-2-fluorobenzene-1-carbohydrazonoyl chloride (980 mg, 2.10 mmol) which was slurried with 4 mL of methanol and the 1-methylpiperidin-4-amine (288 mg, 2.52 mol was added over two minutes. Then 812 mg (6.30 mmol) of diisopropylethylamine was added over two minutes. After ten minutes the reaction was slowly warmed to 60° C. and stirred for 18 hours. The reaction was cooled and 2.0 grams (33.3 mmol) of glacial acetic acid was added over two minutes. The reaction was heated at 65° C. for 18 hours. The reaction was allowed to cool and most of methanol removed under vacuum. Then 40 mL of ethyl acetate and 20 mL of 1.0 N sodium hydroxide solution was added, followed by 1.0 gram of solid sodium hydroxide. Ethyl acetate layer dried and concentrated under vacuum. Insoluble solids from aqueous layer were collected, dissolved with 30 mL dichloromethane. The dichloromethane extract was dried, combined with solid from ethyl acetate extract and concentrated under vacuum to yield was 640 mg of product 60%

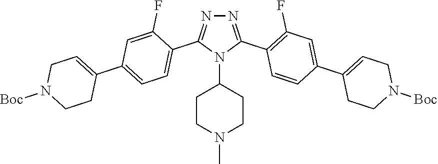 [SMILES: CN1CCC(n2c(-c3ccc(C4=CCN(C(=O)OC(C)(C)C)CC4)cc3F)nnc2-c2ccc(C3=CCN(C(=O)OC(C)(C)C)CC3)cc2F)CC1]

Synthesis of tert-Butyl 4,4′-(4,4′-(4-(1-methylpiperidin-4-yl)-4H-1,2,4-triazole-3,5-diyl)bis(3-fluoro-4,1-phenylene))bis(5,6-dihydropyridine-1(2H)-carboxylate): A flask was charged with 600 mg (1.18 mmol) of -(3,5-bis(4-bromo-2-fluorophenyl)-4H-1,2,4-triazol-4-yl)-1-methylpiperidine, 21 mg (0.094 mmol) of palladium acetate, 98 mg (0.236 mmol) of 2-dicyclohexylphosphino-2′,6′-dimethoxybiphenyl, 870 mg (2.81 mmol) of tert-butyl 4-(4,4,5,5-tetramethyl-1,3,2-dioxaborolan-2-yl)-5,6-dihydropyridine-1(2H)-carboxylate, 9 mL of 1,4-dioxane and 4.4 mL of 2.0 molar potassium carbonate aqueous solution. The flask was swept with nitrogen for five minutes and heated at 95° C. for 18 hours. The reaction was complete by LCMS and the reaction was allowed to cool and diluted with 10 mL water and 10 mL of ethyl acetate added over two minutes. Solids formed quickly and reaction stirred for two hours. Solid collected on a filter and rinsed with 3 mL ethyl acetate and air-dried for 18 hours in the hood to yield 100 mg. The ethyl acetate layer was dried and concentrated under vacuum and combined with filtered solid to yield 550 mg of product (65%).

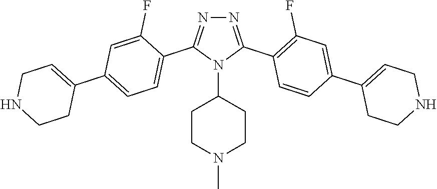 [SMILES: CN1CCC(n2c(-c3ccc(C4=CCNCC4)cc3F)nnc2-c2ccc(C3=CCNCC3)cc2F)CC1]

Synthesis of 4,4′-(4,4′-(4-(1-Methylpiperidin-4-yl)-4H-1,2,4-triazole-3,5-diyl)bis(3-fluoro-4,1-phenylene))bis(1,2,3,6-tetrahydropyridine): A flask was charged with 550 mg (1.07 mmol) of the tert-butyl 4,4′-(4,4′-(4-(1-methylpiperidin-4-yl)-4H-1,2,4-triazole-3,5-diyl)bis(3-fluoro-4,1-phenylene))bis(5,6-dihydropyridine-1(2H)-carboxylate was dissolved with 500 mg (4.63 mmol) of anisole and 5 mL dichloromethane. Then 6 mL (77.9 mmol) of trifluoroacetic acid added over two minutes with stirring and two hours later the reaction was concentrated under vacuum and triturated with two 30 mL portions of ether and ether decanted away to yield a solid in quantitative yield.

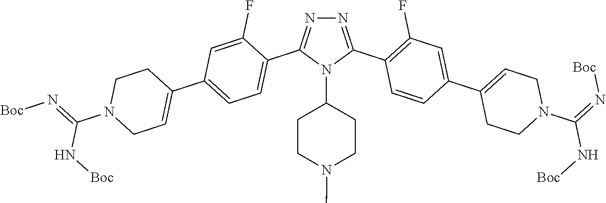 [SMILES: CN1CCC(n2c(-c3ccc(C4=CCN(/C(=N\C(=O)OC(C)(C)C)NC(=O)OC(C)(C)C)CC4)cc3F)nnc2-c2ccc(C3=CCN(/C(=N/C(=O)OC(C)(C)C)NC(=O)OC(C)(C)C)CC3)cc2F)CC1]

Synthesis of tert-Butyl N—[(Z)-(4-{4-[5-(4-{1-[(E)-{[(tert-butoxy)carbonyl]amino)-({[(tert-butoxy)carbonyl]imino})methyl]-1,2,3,6-tetrahydropyridin-4-yl}-2-fluoro phenyl)-4-(1-methylpiperidin-4-yl)-4H-1,2,4-triazol-3-yl]-3-fluorophenyl}-1,2,3,6-tetrahydro pyridin-1-yl}({[(tert-butoxy)carbonyl]imino})methyl]carbamate: A flask was charged with 668 mg (0.90 mmol) of 4,4′-(4,4′-(4-(1-methylpiperidin-4-yl)-4H-1,2,4-triazole-3,5-diyl)bis(3-fluoro-4,1-phenylene))bis(1,2,3,6-tetrahydropyridine) bis trifluoroacetic acid salt and 6 mL N,N-dimethylformamide. Then 681 mg (6.75 mmol) of triethylamine added over two minutes. Then 670 mg (2.16 mmol) of tert-butyl (0.1H-pyrazol-1-yl)methanediylidenedicarbamate was added at once. The reaction was stirred for 18 hours at 23° C. The reaction was diluted with 30 mL ethyl acetate and then washed with two portions of 20 mL of water. Organic phase was dried with sodium sulfate and concentrated under vacuum. Yield was 950 mg.

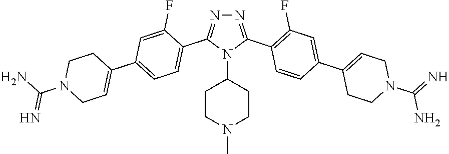 [SMILES: CN1CCC(n2c(-c3ccc(C4=CCN(C(=N)N)CC4)cc3F)nnc2-c2ccc(C3=CCN(C(=N)N)CC3)cc2F)CC1]

### Example 15

Synthesis of 4,4′-(4,4′-(4-(1-Methylpiperidin-4-yl)-4H-1,2,4-triazole-3,5-diyl)bis(3-fluoro-4,1-phenylene))bis(5,6-dihydropyridine-1(2H)-carboximidamide): A flask was charged with 950 mg (0.90 mmol) of tert-butyl N—[(Z)-(4 {4-[5-(4-{1-[(E)-{[(tert-butoxy)-carbonyl]amino}({[(tert-butoxy)carbonyl]imino})-methyl]-1,2,3,6-tetrahydropyridin-4-yl}-2-fluoro phenyl)-4-(1-methylpiperidin-4-yl)-4H-1,2,4-triazol-3-yl]-3-fluorophenyl}-1,2,3,6-tetrahydropyridin-1-yl)({[(tert-butoxy)carbonyl]imino})methyl] carbamate was dissolved with a mixture of 300 mg (2.78 mmol) of anisole and 4 mL of dichloromethane. Then 7 mL (90.9 mmol) of trifluoroacetic acid added over two minutes with stirring and stirred for 18 hours. The LCMS indicted reaction complete and reaction concentrated under vacuum. Solid was dissolved in N,N-dimethylformamide and was purified by prep HPLC. Yield was 332 mg (40%). ¹H NMR. (300 MHz, DMSO-d₆) δ=7.67-7.52 (m, 12H), 6.51 (br t, J=3.2 Hz, 2H), 4.15-4.10 (m, 4H), 3.70-3.64 (m, 6H), 3.29 (br d, J=11.2 Hz, 2H), 2.97 (br d, J=11.6 Hz, 2H), 2.64 (br t, J=4.6 Hz, 4H), 2.58-2.55 (m, 2H), 2.06 (br d, J=12.2 Hz, 2H), 1.83 (br d J=13.1 Hz, 2H). LC/MS method A: R_{f}=2.27 mins., (M+H)⁺=602, 301, purity >95%.

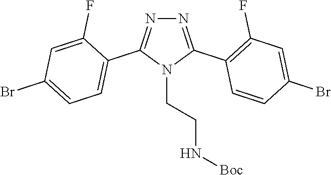 [SMILES: CC(C)(C)OC(=O)NCCn1c(-c2ccc(Br)cc2F)nnc1-c1ccc(Br)cc1F]

Synthesis of tert-Butyl 2-(3,5-bis(4-bromo-2-fluorophenyl)-4H-1,2,4-triazol-4-yl)ethyl carbamate: A flask was charged with (Z)-4-bromo-N-[(1Z)-(4-bromo-2-fluorophenyl)(chloro)methylidene]-2-fluorobenzene-1-carbohydrazonoyl chloride (940 mg, 2.00 mmol) was slurried with 4 mL of methanol and the tert-butyl 2-aminoethylcarbamate (335 mg, 2.1 mol) was added at once. Then 700 mg (5.40 mmol) of diisopropylethylamine was added. After 30 minutes the reaction was slowly warmed to 65° C. for three days. The methanol was removed under vacuum and 7 mL N,N-dimethylformamide added. The reaction was heated at 80° C. for 18 hours. The reaction was cooled and 60 mL ethyl acetate and 20 mL water added. The organic phase was dried and concentrated under vacuum. Yield was 900 mg (62%).

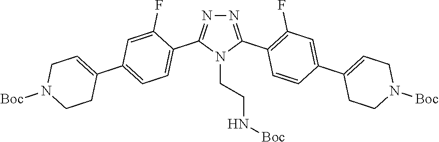 [SMILES: CC(C)(C)OC(=O)NCCn1c(-c2ccc(C3=CCN(C(=O)OC(C)(C)C)CC3)cc2F)nnc1-c1ccc(C2=CCN(C(=O)OC(C)(C)C)CC2)cc1F]

Synthesis of tert-Butyl 4,4′-(4,4′-(4-(2-(tert-butoxycarbonylamino)ethyl)-4H-1,2,4-triazole-3,5-diyl)bis(3-fluoro-4,1-phenylene))bis(5,6-dihydropyridine-1(2H)-carboxylate: A flask was charged with 900 mg (1.230 mmol) of tert-butyl 2-(3,5-bis(4-bromo-2-fluorophenyl)-4H-1,2,4-triazol-4-yl)ethylcarbamate and tert-butyl 4-(4,4,5,5-tetramethyl-1,3,2-dioxaborolan-2-yl)-5,6-dihydropyridine-1(2H)-carboxylate, 28 mg (0.126 mmol) of palladium acetate, 130 mg (0.316 mmol) of 2-dicyclohexylphosphino-2′,6′-dimethoxybiphenyl, 1180 mg (3.79 mmol) of tert-butyl 4-(4,4,5,5-tetramethyl-1,3,2-dioxaborolan-2-yl)-5,6-dihydropyridine-1(2H)-carboxylate, 10 mL of 1,4-dioxane and 6 mL of 2.0 molar potassium carbonate aqueous solution. The flask was swept with nitrogen for five minutes and heated at 95° C. for 18 hours. The reaction was complete by LCMS and the reaction was allowed to cool and diluted with 30 mL of ethyl acetate and washed with two 20 mL portions of water, dried and concentrated under vacuum. The concentrate was dissolved with 8 mL acetic acid. The reaction was split in two roughly equal portions. One half of acetic acid solution was diluted with 6 mL (77.9 mmol) of trifluoroacetic acid and stirred for 18 hours at 23° C. and then heated at 50° C. for three hours and was discarded because of complexity. The other half was heated at 80° C. for 18 hours and then concentrated under vacuum and used without further purification. Yield was 50% (470 mg).

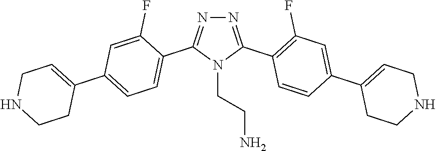 [SMILES: NCCn1c(-c2ccc(C3=CCNCC3)cc2F)nnc1-c1ccc(C2=CCNCC2)cc1F]

Synthesis of 2-(3,5-bis(2-Fluoro-4-(1,2,3,6-tetrahydropyridin-4-yl)phenyl)-4H-1,2,4-triazol-4-yl)ethanamine: The tert-butyl 4,4′-(4,4′-(4-(2-(tert-butoxycarbonylamino) ethyl)-4H-1,2,4-triazole-3,5-diyl)bis(3-fluoro-4,1-phenylene))bis(5,6-dihydropyridine-1(2H)-carboxylate) (470 mg, 0.616 mmol) was dissolved with 400 mg (3.71 mmol) of anisole and 4 mL dichloromethane. Then 6 mL (77.9 mmol) of trifluoroacetic acid added over five minutes with stirring. Two hours later the reaction was concentrated under vacuum and triturated with two 30 mL portions of ether and ether decanted away to yield a solid in quantitative yield.

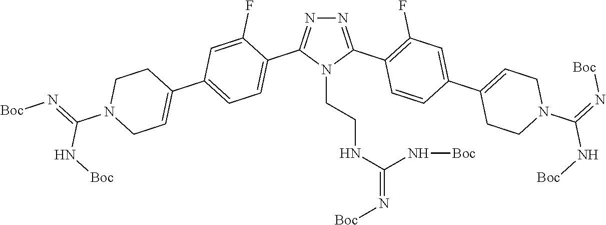 [SMILES: CC(C)(C)OC(=O)/N=C(/NC(=O)OC(C)(C)C)N1CC=C(c2ccc(-c3nnc(-c4ccc(C5=CCN(/C(=N/C(=O)OC(C)(C)C)NC(=O)OC(C)(C)C)CC5)cc4F)n3CCN/C(=N\C(=O)OC(C)(C)C)NC(=O)OC(C)(C)C)c(F)c2)CC1]

Synthesis of tert-Butyl N—[(Z)-{[2-(3-{4-[1-({[(tert-butoxy)carbonyl]-amino}({[tert-butoxy)carbonyl]imino})methyl)-1,2,3,6-tetrahydropyridin-4-yl]-2-fluoro phenyl}-5-{4-[1-({[(tert-butoxy)carbonyl]amino}[(4,4-dimethylpent-1-en-2-yl)iminomethyl)-1,2,3,6-tetrahydropyridin-4-yl]-2-fluorophenyl}-4H-1,2,4-triazol-4-yl)ethyl]amino}({[(tert-butoxy)carbonyl]imino})methyl]carbamate: A flask was charged with 400 mg (0.5 mmol) of from 2-(3,5-bis(2-fluoro-4-(1,2,3,6-tetrahydropyridin-4-yl)phenyl)-4H-1,2,4-triazol-4-yl) ethanamine tris trifluoroacetic acid salt and 4 mL N,N-dimethylformamide. Then 530 mg (5.25 mmol) of triethylamine was added over two minutes. Then 541 mg (1.75 mmol) of tert-butyl (1H-pyrazol-1-yl)methanediylidene dicarbamate was added at once. The reaction stirred for 18 hours at 23° C. The reaction was diluted 40 mL ethyl acetate and then washed with three portions of 20 mL of water. Organic phase was dried with sodium sulfate and concentrated under vacuum. The product was purified by normal phase chromatography, loaded as dichloromethane solution, eluted with ethyl acetate/dichloromethane with step gradient from 0 to 100% to elute product. Yield was 240 mg of product (40%).

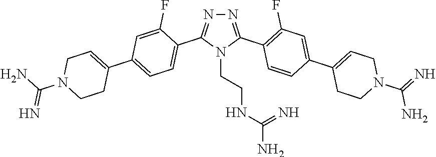 [SMILES: N=C(N)NCCn1c(-c2ccc(C3=CCN(C(=N)N)CC3)cc2F)nnc1-c1ccc(C2=CCN(C(=N)N)CC2)cc1F]

### Example 16

Synthesis of 4,4′-(4,4′-(4-(2-guanidinoethyl)-4H-1,2,4-triazole-3,5-diyl)bis(3-fluoro-4,1-phenylene))bis(5,6-dihydropyridine-1(2H)-carboximidamide): A flask was charged with 240 mg (0.20 mmol) of tert-butyl 4,4′-(4,4′-(4-(2-(3-(tert-butoxycarbonyl) guanidino) ethyl)-4H-1,2,4-triazole-3,5-diyl)bis(3-fluoro-4,1-phenylene))bis(5,6-dihydropyridine-4,1(2H)-diyl) bis(methane-1-yl-1,1-diylidene)tetracarbamate was dissolved with a mixture of 300 mg (2.78 mmol) of anisole and 4 mL of dichloromethane. Then 6 mL (77.9 mmol) of trifluoroacetic acid was added over two minutes with stirring and stirred for 18 hours. The next day LCMS indicted reaction was complete and reaction concentrated under vacuum to yield an oil which was dissolved in N,N-dimethylformamide and was purified by prep HPLC. Yield was 103 mg (55%). ¹H NMR (300 MHz, DMSO-d₆) δ=7.68-7.65 (m, 2H), 7.62 (d, J=5.2 Hz, 2H), 7.57 (d, J=1.6 Hz, 2H), 7.54-7.45 (m, 9H), 6.47 (s, 2H), 4.12 (s, 4H), 4.00 (s, 2H), 3.64 (br t, J=5.6 Hz, 4H), 2.97 (br d, J=5.5 Hz, 2H), 2.64 (br s, 4H). LC/MS method A: R_{f}=2.33 mins., (M+H)⁺=590, 296, purity >95%.

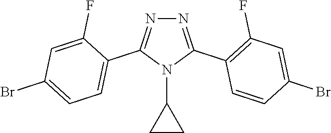 [SMILES: Fc1cc(Br)ccc1-c1nnc(-c2ccc(Br)cc2F)n1C1CC1]

Synthesis of 3,5-bis(4-Bromo-2-fluorophenyl)-4-cyclopropyl-4H-1,2,4-triazole: A flask was charged with the crude (Z)-4-bromo-N-[(1Z)-(4-bromo-2-fluorophenyl)(chloro)methylidene]-2-fluorobenzene-1-carbohydrazonoyl chloride (1.0 grams, 2.12 mmol) was slurried with 5 mL of methanol and the cyclopropylamine (0.4 g, 7.01 mmol), 2.0 M in methanol was added over five minutes followed by 0.19 grams (3.19 mmol) of glacial acetic acid was added over five minutes. The reaction was heated at 70° C. for 18 hours. The reaction was allowed to cool and most of methanol removed under vacuum. Then 40 mL of water added over several minutes. One hour later the solid was collected, air dried for 15 minutes and rinsed with ten mL of water, ten mL of ether and air dried in the hood for several hours. Yield was 0.86 grams (88%)

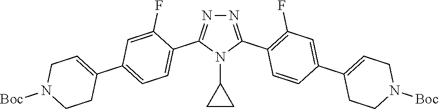 [SMILES: CC(C)(C)OC(=O)N1CC=C(c2ccc(-c3nnc(-c4ccc(C5=CCN(C(=O)OC(C)(C)C)CC5)cc4F)n3C3CC3)c(F)c2)CC1]

Synthesis of tert-Butyl 4,4′-(4,4′-(4-cyclopropyl-4H-1,2,4-triazole-3,5-diyl)bis(3-fluoro-4,1-phenylene))bis(5,6-dihydropyridine-1(2H)-carboxylate): A flask was charged with 300 mg (0.65 mmol) of 3,5-bis(4-bromo-2-fluorophenyl)-4-cyclopropyl-4H-1,2,4-triazole, 11.8 mg (0.05 mmol) of palladium acetate, 54 mg (0.13 mmol) of 2-dicyclohexylphosphino-2′,6′-dimethoxybiphenyl, 489 mg (1.58 mmol) of tert-butyl 4-(4,4,5,5-tetramethyl-1,3,2-dioxaborolan-2-yl)-5,6-dihydropyridine-1(2H)-carboxylate, 2.0 mL of 1,4-dioxane and 1.0 mL of 2.0 molar potassium carbonate aqueous solution. The flask was swept with nitrogen for five minutes and heated at 95° C. for 18 hours. Then the reaction was complete by LCMS and the reaction was allowed to cool and diluted with 4 mL water and 10 mL of ethyl acetate added over ten minutes. Solids formed quickly and reaction stirred for two hours. Solid collected on a filter and rinsed with 30 mL ethyl acetate and air-dried for 18 hours in the hood to yield 198 mg (42%).

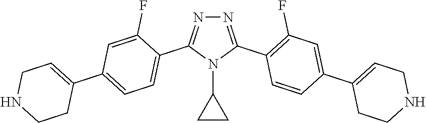 [SMILES: Fc1cc(C2=CCNCC2)ccc1-c1nnc(-c2ccc(C3=CCNCC3)cc2F)n1C1CC1]

Synthesis of 4,4′-(4,4′-(4-Cyclopropyl-4H-1,2,4-triazole-3,5-diyl)bis(3-fluoro-4,1-phenylene))bis(1,2,3,6-tetrahydropyridine): di-tert-butyl 4,4′-((4-cyclopropyl-4H-1,2,4-triazole-3,5-diyl)bis(3-fluoro-4,1-phenylene))bis(5,6-dihydropyridine-1(2H)-carboxylate) 200 mg (0.30 mmol) was slurried with 0.5 mL of anisole and 1.5 mL dichloromethane. Then 1.0 mL (40 mmol) of trifluoroacetic acid added over five minutes with stirring. The reaction was concentrated under vacuum and triturated with two 5 mL portions of ether and ether decanted away to yield a solid in quantitative yield.

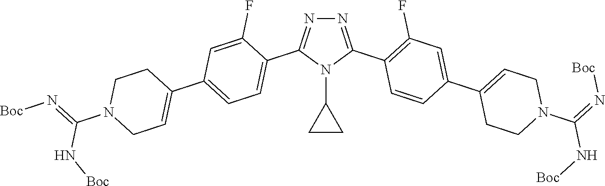 [SMILES: CC(C)(C)OC(=O)/N=C(/NC(=O)OC(C)(C)C)N1CC=C(c2ccc(-c3nnc(-c4ccc(C5=CCN(/C(=N/C(=O)OC(C)(C)C)NC(=O)OC(C)(C)C)CC5)cc4F)n3C3CC3)c(F)c2)CC1]

Synthesis of tert-Butyl (4,4′-(4,4′-(4-cyclopropyl-4H-1,2,4-triazole-3,5-diyl)bis(3-fluoro-4,1-phenylene))bis(5,6-dihydropyridine-4,1(2H)-diyl))bis(methane-1-yl-1,1-diylidene) tetracarbamate: A flask was charged with 82.6 mg (0.17 mmol) of 4,4′-((4-cyclopropyl-4H-1,2,4-triazole-3,5-diyl)bis(3-fluoro-4,1-phenylene))bis(1,2,3,6-tetrahydropyridine) bis trifluoroacetic acid salt and 1.0 mL N,N-dimethylformamide. Then 109 mg (1.07 mmol) of triethylamine added. Then 11.1 mg (0.35 mmol) of tert-butyl (1H-pyrazol-1-yl)methanediylidenedicarbamate was added. The reaction slurry was warmed at 40° C. for 18 hours. The reaction was diluted with 20 mL ethyl acetate and then washed with four portions of 5 mL of water. Organic phase was dried with sodium sulfate and concentrated under vacuum. The product was purified by normal phase chromatography, loaded as dichloromethane solution, eluted with (30% acetonitrile/dichloromethane)/dichloromethane with step gradient from 0 to 100% to elute impurities. Then the expected product eluted off with (20% MeOH/dichloromethane)/dichloromethane step gradient from 0 to 0.100%. Yield was 65.8 mg of product (98%).

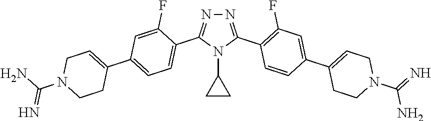 [SMILES: N=C(N)N1CC=C(c2ccc(-c3nnc(-c4ccc(C5=CCN(C(=N)N)CC5)cc4F)n3C3CC3)c(F)c2)CC1]

### Example 17

Synthesis of 4,4′-(4,4′-(4-Cyclopropyl-4H-1,2,4-triazole-3,5-diyl)bis(3-fluoro-4,1-phenylene))bis(5,6-dihydropyridine-1(2H)-carboximidamide): A solution of di-tert-butyl ((4,4′-((4-cyclopropyl-4H-1,2,4-triazole-3,5-diyl)bis(3-fluoro-4,1-phenylene))bis(5,6-dihydropyridine-4,1(2H)-diyl))bis(((tert-butoxycarbonyl)amino)methanylylidene)) dicarbamate (25.6 mg, 0.17 mmol) was dissolved with a mixture of 0.5 mL of anisole and 4 mL of dichloromethane. Then 2 mL of trifluoroacetic acid added over two minutes with stirring and stirred for 18 hours. The next day LCMS indicted reaction complete and reaction concentrated under vacuum. Oil was triturated with two 10 mL portions of ether and ether decanted away to yield a solid product. Yield was 44 mg (100%). ¹H NMR (300 MHz, DMSO-d₆) ppm 7.39-7.66 (m, 12H) 6.50 (br. s., 2H) 4.07-4.24 (m, 4H) 3.31-3.45 (m, 5H) 2.66 (br. s., 4H) 0.55-0.75 (m, 2H) 0.17-0.37 (m, 2H); LC/MS method A: R_{f}=3.10 mins., (M+H)+=544, purity >95%.

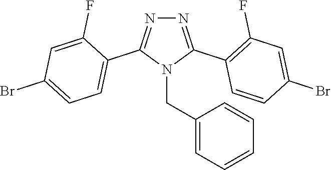 [SMILES: Fc1cc(Br)ccc1-c1nnc(-c2ccc(Br)cc2F)n1Cc1ccccc1]

Synthesis of 4-Benzyl-3,5-bis(4-bromo-2-fluorophenyl)-4H-1,2,4-triazole: A flask was charged with the crude (Z)-4-bromo-N-[(1Z)-(4-bromo-2-fluorophenyl)(chloro)methylidene]-2-fluorobenzene-1-carbohydrazonoyl chloride (1.0 grams, 2.12 mmol) was slurried with 5 mL, of methanol and the benzylamine (0.75 g, 7.01 mmol), was added over five minutes followed by 0.19 grams (3.19 mmol) of glacial acetic acid was added over five minutes. The reaction was heated at 70° C. for 18 hours. The reaction was allowed to cool and most of methanol removed under vacuum. Then 40 mL of water added over several minutes. One hour later the solid was collected, air dried for 15 minutes and rinsed with ten mL of water, ten mL of ether and air dried in the hood for several hours. Yield was 1.01 grams (94%).

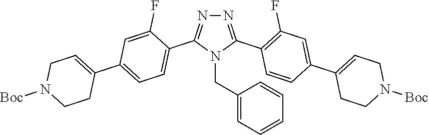 [SMILES: CC(C)(C)OC(=O)N1CC=C(c2ccc(-c3nnc(-c4ccc(C5=CCN(C(=O)OC(C)(C)C)CC5)cc4F)n3Cc3ccccc3)c(F)c2)CC1]

Synthesis of tert-Butyl 4,4′′-(4,4′-(4-benzyl-4H-1,2,4-triazole-3,5-diyl)bis(3-fluoro-4,1-phenylene))bis(5,6-dihydropyridine-1(2H)-carboxylate): A flask was charged with 300 mg (0.65 mmol) of 4-benzyl-3,5-bis(4-bromo-2-fluorophenyl)-4H-1,2,4-triazole, 10.68 mg (0.05 mmol) of palladium acetate, 48.7 mg (0.11 mmol) of 2-dicyclohexylphosphino-2′,6′-dimethoxybiphenyl, 440 mg (1.42 mmol) of tert-butyl 4-(4,4,5,5-tetramethyl-1,3,2-dioxaborolan-2-yl)-5,6-dihydropyridine-1(2H)-carboxylate, 2.0 mL of 1,4-dioxane and 1.0 mL of 2.0 molar potassium carbonate aqueous solution. The flask was swept with nitrogen for five minutes and heated at 95° C. for 18 hours. Then the reaction was complete by LCMS and the reaction was allowed to cool and diluted with 4 mL water and 10 mL of ethyl acetate added over ten minutes. Solids formed quickly and reaction stirred for two hours. Solid collected on a filter and rinsed with 30 mL ethyl acetate and air-dried for 18 hours in the hood to yield 211 mg (50%).

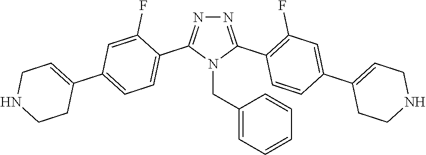 [SMILES: Fc1cc(C2=CCNCC2)ccc1-c1nnc(-c2ccc(C3=CCNCC3)cc2F)n1Cc1ccccc1]

Synthesis of 4,4′-(4,4′-(4-Benzyl-4H-1,2,4-triazole-3,5-diyl)bis(3-fluoro-4,1-phenylene)) bis(1,2,3,6-tetrahydropyridine): Synthesis of 4,4′-((4-benzyl-4H-1,2,4-triazole-3,5-diyl)bis(3-fluoro-4,1-phenylene))bis(1,2,3,6-tetrahydropyridine) bis trifluoroacetic acid salt: di-tert-butyl 4,4′-((4-benzyl-4H-1,2,4-triazole-3,5-diyl)bis(3-fluoro-4,1-phenylene))bis(5,6-dihydropyridine-1(2H)-carboxylate) 200 mg (0.28 mmol) was slurried with 0.5 mL of anisole and 1.5 mL dichloromethane. Then 1.0 mL (40 mmol) of trifluoroacetic acid added over five minutes with stirring. The reaction was concentrated under vacuum and triturated with two 5 mL portions of ether and ether decanted away to yield a solid in quantitative yield.

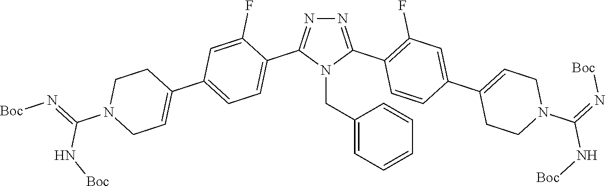 [SMILES: CC(C)(C)OC(=O)/N=C(/NC(=O)OC(C)(C)C)N1CC=C(c2ccc(-c3nnc(-c4ccc(C5=CCN(/C(=N/C(=O)OC(C)(C)C)NC(=O)OC(C)(C)C)CC5)cc4F)n3Cc3ccccc3)c(F)c2)CC1]

Synthesis of tert-Butyl (4,4′-(4,4′-(4-benzyl-4H-1,2,4-triazole-3,5-diyl)bis(3-fluoro-4,1-phenylene))bis(5,6-dihydropyridine-4,1(2H)-diyl))bis(methane-1-yl-1,1-diylidene) tetracarbamate: A flask was charged with 80 mg (0.15 mmol) of 4,4′-((4-benzyl-4H-1,2,4-triazole-3,5-diyl)bis(3-fluoro-4,1-phenylene))bis(1,2,3,6-tetrahydropyridine) bis trifluoroacetic acid salt and 1.0 mL N,N-dimethylformamide. Then 95 mg (0.94 mmol) of triethylamine added. Then 146 mg (0.47 mmol) of tert-butyl (1H-pyrazol-1-yl)methanediylidenedicarbamate was added. The reaction slurry was warmed at 40° C. for 18 hours. The reaction was diluted with 20 mL ethyl acetate and then washed with four portions of 5 mL of water. Organic phase was dried with sodium sulfate and concentrated under vacuum. The product was purified by normal phase chromatography, loaded as dichloromethane solution, eluted with (30% acetonitrile/dichloromethane)/dichloromethane with step gradient from 0 to 100% to elute impurities. Then the expected product eluted off with (20% MeOH/dichloromethane)/dichloromethane step gradient from 0 to 100%. Yield was 87 mg of product (55%).

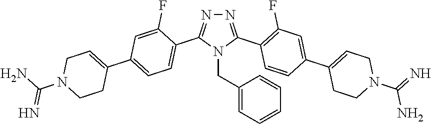 [SMILES: N=C(N)N1CC=C(c2ccc(-c3nnc(-c4ccc(C5=CCN(C(=N)N)CC5)cc4F)n3Cc3ccccc3)c(F)c2)CC1]

### Example 18

Synthesis of 4,4′-(4,4′-(4-Benzyl-4H-1,2,4-triazole-3,5-diyl)bis(3-fluoro-4,1-phenylene)) bis(5,6-dihydropyridine-1(2H)-carboximidamide: A solution of di-tert-butyl ((4,4′-((4-benzyl-4H-1,2,4-triazole-3,5-diyl)bis(3-fluoro-4,1-phenylene))bis(5,6-dihydropyridine-4,1(2H)-diyl))bis(((tert-butoxycarbonyl)amino)methanylylidene)) dicarbamate (87.6 mg, 0.087 mmol) was dissolved with a mixture of 0.5 mL of anisole and 4 mL of dichloromethane. Then 2 mL of trifluoroacetic acid added over two minutes with stirring and stirred for 18 hours. The next day LCMS indicted reaction complete and reaction concentrated under vacuum. Oil was triturated with two 10 mL portions of ether and ether decanted away to yield a solid product. ¹H NMR (300 MHz, DMSO-d₆) δ ppm 7.38-7.66 (m, 13H) 7.01-7.17 (m, 4H) 6.58-6.72 (m, 2H) 5.02-5.24 (m, 2H) 3.93-4.20 (m, 4H) 3.2 (s, 4H), 2.50-2.65 (m, 4H); LC/MS method A: R_{f}=3.36 mins., (M+H)⁺=594, purity >95%.

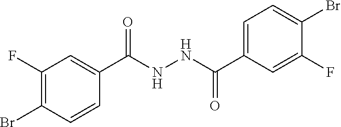 [SMILES: O=C(NNC(=O)c1ccc(Br)c(F)c1)c1ccc(Br)c(F)c1]

4-Bromo-N′-(4-bromo-3-fluorobenzoyl)-3-fluorobenzohydrazide was prepared from hydrazine and 4-bromo-3-fluorobenzoyl chloride.

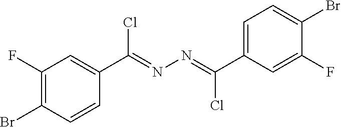 [SMILES: Fc1cc(/C(Cl)=N/N=C(\Cl)c2ccc(Br)c(F)c2)ccc1Br]

(1,N)-4-Bromo-N′-((4-bromo-3-fluorophenyl)chloromethylene)-3-fluorobenzo hydrazonoyl chloride was prepared from 4-bromo-N-(4-bromo-3-fluorobenzoyl)-3-fluorobenzo hydrazide.

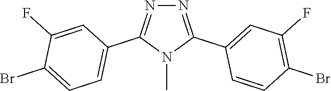 [SMILES: Cn1c(-c2ccc(Br)c(F)c2)nnc1-c1ccc(Br)c(F)c1]

3,5-Bis(4-Bromo-3-fluorophenyl)-4-methyl-4H-1,2,4-triazole was prepared from (1,N)-4-bromo-N′-((4-bromo-3-fluorophenyl)chloromethylene)-3-fluorobenzo hydrazonoyl chloride and methylamine.

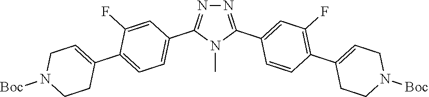 [SMILES: Cn1c(-c2ccc(C3=CCN(C(=O)OC(C)(C)C)CC3)c(F)c2)nnc1-c1ccc(C2=CCN(C(=O)OC(C)(C)C)CC2)c(F)c1]

tert-Butyl 4,4-(4,4-(4-methyl-4H-1,2,4-triazole-3,5-diyl)bis(2-fluoro-4,1-phenylene))bis(5,6-dihydropyridine-1(2H)-carboxylate was prepared from 3,5-bis(4-bromo-3-fluorophenyl)-4-methyl-4H-1,2,4-triazole.

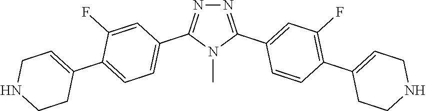 [SMILES: Cn1c(-c2ccc(C3=CCNCC3)c(F)c2)nnc1-c1ccc(C2=CCNCC2)c(F)c1]

4,4′-(4,4′-(4-Methyl-4H-1,2,4-triazole-3,5-diyl)bis(2-fluoro-4,1-phenylene))bis (1,2,3,6-tetrahydropyridine) was prepared from tert-butyl 4,4′-(4,4′-(4-methyl-4H-1,2,4-triazole-3,5-diyl)bis(2-fluoro-4,1-phenylene))bis(5,6-dihydropyridine-1(2H)-carboxylate.

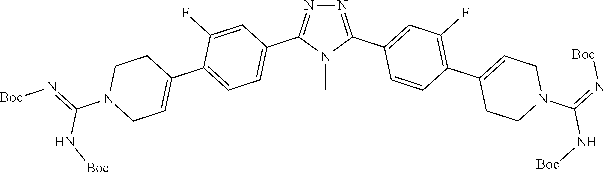 [SMILES: Cn1c(-c2ccc(C3=CCN(/C(=N\C(=O)OC(C)(C)C)NC(=O)OC(C)(C)C)CC3)c(F)c2)nnc1-c1ccc(C2=CCN(/C(=N/C(=O)OC(C)(C)C)NC(=O)OC(C)(C)C)CC2)c(F)c1]

tert-Butyl (4,4′-(4,4-(4-methyl-4H-1,2,4-triazole-3,5-diyl)bis(2-fluoro-4,1-phenylene))bis(5,6-dihydropyridine-4,1(2H)-diyl))bis(methane-1-yl-1,1-diylidene) tetracarbamate was prepared from 4,4′-(4,4′-(4-methyl-4H-1,2,4-triazole-3,5-diyl)bis(2-fluoro-4,1-phenylene))bis(1,2,3,6-tetrahydropyridine).

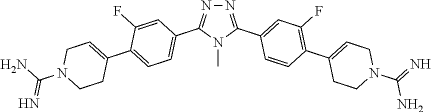 [SMILES: Cn1c(-c2ccc(C3=CCN(C(=N)N)CC3)c(F)c2)nnc1-c1ccc(C2=CCN(C(=N)N)CC2)c(F)c1]

### Example 19

4,4′-(4,4-(4-Methyl-4H-1,2,4-triazole-3,5-diyl)bis(2-fluoro-4,1-phenylene)) bis(5,6-dihydropyridine-1(2H)-carboximidamide) was prepared from tert-butyl (4,4′-(4,4′-(4-methyl-4H-1,2,4-triazole-3,5-diyl)bis(2-fluoro-4,1-phenylene))bis(5,6-dihydropyridine-4,1-(2H)-diyl))bis(methane-1-yl-1,1-diylidene)tetracarbamate. ¹H NMR (300 MHz, METHANOL-d₄) δ=7.66-7.61 (m, 4H), 7.58 (d, J=1.3 Hz, 1H), 7.36-7.32 (m, 1H), 6.20-6.17 (m, 2H), 4.20-4.16 (m, 3H), 3.80 (br s, 2H), 3.72 (t, J=5.6 Hz, 2H), 3.22 (t, J=1.9 Hz, 2H), 2.77-2.70 (m, 4H). LC/MS method A: R_{f}=2.89 mins., (M+H)⁺=518, purity >95%.

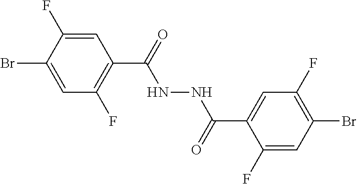 [SMILES: O=C(NNC(=O)c1cc(F)c(Br)cc1F)c1cc(F)c(Br)cc1F]

Synthesis of 4-Bromo-N′-(4-bromo-2,5-difluorobenzoyl)-2,5-difluorobenzohydrazide: A flask was charged with 50 mL of chloroform and cooled in an ice water bath and 98.0 mg (3.9 mmol) of hydrazine monohydrate (0.6 g, 1.92 mmol) of N,N-diisopropylethylamine was added. A solution of 1.0 g (3.91 mmol) of 4-bromo-2-fluorobenzoyl chloride in 50 mL of chloroform was added over one hour and stirred for 18 hours at 23° C. Removed chloroform under vacuum and stir with 150 mL of water for 18 hours after brief sonication. Solids isolated by decantation/filtration. Filter cake/flask rinsed with 30 mL acetonitrile. Solids from filter added to solids from flask and two 120 mL portions of acetonitrile added and removed under vacuum to dry solid. Yield was 1.7 grams (92%).

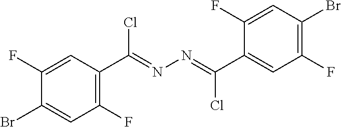 [SMILES: Fc1cc(/C(Cl)=N/N=C(\Cl)c2cc(F)c(Br)cc2F)c(F)cc1Br]

Synthesis of (Z)-4-bromo-N-[(1Z)-(4-bromo-2,5-difluorophenyl)(chloro) methylidene]-2,5-difluorobenzene-1-carbohydrazonoyl chloride: A 250 mL flask was charged with 0.1.7 g (3.63 mmol) of 4-bromo-N-(4-bromo-2,5-difluorobenzoyl)-2,5-difluorobenzohydrazide. The solid was slurried with 100 mL toluene and briefly sonicated. 2.27 g (10.9 mmol) of PCl₅ was added in three roughly equal portions. The reaction was heated to 100° C. for 18 hours. The reaction was cooled and concentrated under vacuum to yield a solid. The reaction was quenched by addition of wet ice and water (about 50 grams). After a two-hour age water removed by decantation and two 100 mL portions of acetonitrile added and removed under vacuum to remove water. Yield 0.96 grams (52%).

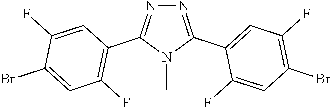 [SMILES: Cn1c(-c2cc(F)c(Br)cc2F)nnc1-c1cc(F)c(Br)cc1F]

Synthesis of 3,5-Bis(4-bromo-2,5-difluorophenyl)-4-methyl-4H-1,2,4-triazole chloride: A flask was charged with (Z)-4-bromo-N-[(1Z)-(4-bromo-2,5-difluorophenyl) (chloro)methyl idene]-2,5-difluorobenzene-1-carbohydrazonoyl chloride (962 grams, 2.04 mmol) was slurried with 5 mL of methanol and the methylamine (0.962 g, 6.7 mmol), was added over five minutes followed by 0.183 grams (3.06 mmol) of glacial acetic acid was added over five minutes. The reaction was heated at 70° C. for 18 hours. The reaction was allowed to cool and most of methanol removed under vacuum. Then 40 mL of water added over several minutes. One hour later the solid was collected, air dried for 15 minutes and rinsed with ten mL of water, ten mL of ether and air dried in the hood for several hours. Yield was 1.10 grams (96%).

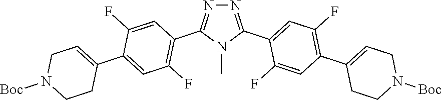 [SMILES: Cn1c(-c2cc(F)c(C3=CCN(C(=O)OC(C)(C)C)CC3)cc2F)nnc1-c1cc(F)c(C2=CCN(C(=O)OC(C)(C)C)CC2)cc1F]

Synthesis of tert-Butyl 4,4′-(4,4′-(4-methyl-4H-1,2,4-triazole-3,5-diyl)bis(2,5-difluoro-4,1-phenylene))bis(5,6-dihydropyridine-1(2H)-carboxylate)): A flask was charged with 1101 mg (2.36 mmol) of 3,5-bis(4-bromo-2,5-difluorophenyl)-4-methyl-4H-1,2,4-triazole, 42.5 mg (5.68 mmol) of palladium acetate, 194.3 mg (0.47 mmol) of 2-dicyclohexylphosphino-2′,6′-dimethoxybiphenyl, 1756 mg (5.68 mmol) of tert-butyl 4-(4,4,5,5-tetramethyl-1,3,2-dioxaborolan-2-yl)-5,6-dihydropyridine-1(2H)-carboxylate, 2.0 mL of 1,4-dioxane and 1.0 mL of 2.0 molar potassium carbonate aqueous solution. The flask was swept with nitrogen for five minutes and heated at 95° C. for 18 hours. Then the reaction was complete by LCMS and the reaction was allowed to cool and diluted with 4 mL water and 10 mL of ethyl acetate added over ten minutes. Solids formed quickly and reaction stirred for two hours. Solid collected on a filter and rinsed with 30 mL ethyl acetate and air-dried for 18 hours in the hood to yield 431 mg (27%).

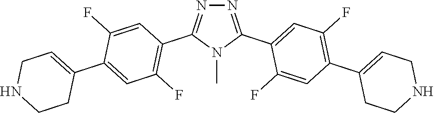 [SMILES: Cn1c(-c2cc(F)c(C3=CCNCC3)cc2F)nnc1-c1cc(F)c(C2=CCNCC2)cc1F]

Synthesis of 4,4-(4,4-(4-Methyl-4H-1,2,4-triazole-3,5-diyl)bis(2,5-difluoro-4,1-phenylene))bis(1,2,3,6-tetrahydropyridine): di-tert-butyl 4,4′-((4-methyl-4H-1,2,4-triazole-3,5-diyl)bis(2,5-difluoro-4,1-phenylene))bis(5,6-dihydropyridine-1(2H)-carboxylate) 400 mg (0.45 mmol) was slurried with 0.5 mL of anisole and 1.5 mL dichloromethane. Then 1.0 mL (40 mmol) of trifluoroacetic acid added over five minutes with stirring. The reaction was concentrated under vacuum and triturated with two 5 mL portions of ether and ether decanted away to yield a solid in quantitative yield.

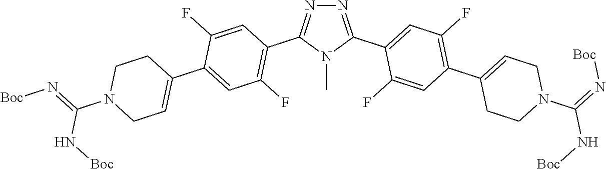 [SMILES: Cn1c(-c2cc(F)c(C3=CCN(/C(=N\C(=O)OC(C)(C)C)NC(=O)OC(C)(C)C)CC3)cc2F)nnc1-c1cc(F)c(C2=CCN(/C(=N/C(=O)OC(C)(C)C)NC(=O)OC(C)(C)C)CC2)cc1F]

Synthesis of tert-Butyl (4,4′-(4,4′-(4-methyl-4H-1,2,4-triazole-3,5-diyl)bis(2,5-difluoro-4,1-phenylene))bis(5,6-dihydropyridine-4,1(2H)-diyl))bis(methane-1-yl-1,1-diylidene)tetra carbamate): A flask was charged with 200 mg (0.42 mmol) of 4,4′-((4-methyl-4H-1,2,4-triazole-3,5-diyl)bis(2,5-difluoro-4,1-phenylene))bis(1,2,3,6-tetrahydropyridine) bis trifluoroacetic acid salt and 1.0 mL N,N-dimethylformamide. Then 258 mg (2.55 mmol) of triethylamine added. Then 396 mg (1.2 mmol) of tert-butyl. (1H-pyrazol-1-yl)methanediylidenedicarbamate was added. The reaction slurry was warmed at 40° C.; for 18 hours. The reaction was diluted with 20 mL ethyl acetate and then washed with four portions of 5 mL of water. Organic phase was dried with sodium sulfate and concentrated under vacuum. The product was purified by normal phase chromatography, loaded as dichloromethane solution, eluted with (30% acetonitrile/dichloromethane)/dichloromethane with step gradient from 0 to 100% to elute impurities. Then the expected product eluted off with (20% MeOH/dichloromethane)/dichloromethane step gradient from 0 to 100%. Yield was 106 mg (26%).

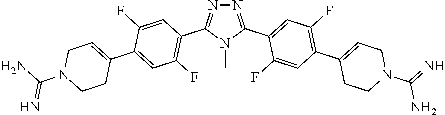 [SMILES: Cn1c(-c2cc(F)c(C3=CCN(C(=N)N)CC3)cc2F)nnc1-c1cc(F)c(C2=CCN(C(=N)N)CC2)cc1F]

### Example 20

Synthesis of 4,4′-(4,4′-(4-Methyl-4H-1,2,4-triazole-3,5-diyl)bis(2,5-difluoro-4,1-phenylene))bis(5,6-dihydropyridine-1(2H)-carboximidamide); A solution of di-tert-butyl ((4,4′-((4-methyl-4H-1,2,4-triazole-3,5-diyl)bis(2,5-difluoro-4,1-phenylene))bis(5,6-dihydropyridine-4,1(2H)-diyl))bis(((tert-butoxycarbonyl)amino)methanylylidene)) dicarbamate (106 mg, 0.11 mmol) was dissolved with a mixture of 0.5 mL of anisole and 4 mL of dichloromethane. Then 2 mL of trifluoroacetic acid added over two minutes with stirring and stirred for 18 hours. The next day LCMS indicted reaction complete and reaction concentrated under vacuum. Oil was triturated with two 10 mL portions of ether and ether decanted away to yield a solid product. Yield was 60 mg (99%). ¹H NMR (300 MHz, DMSO-d₆) δ ppm 7.71-7.53 (m, 4H), 7.44 (s, 6H), 6.37-6.17 (m, 2H), 4.24-3.94 (m, 3H), 3.64 (m, 4H); 3.53-3.46 (m, 4H), 2.73-2.55 (m, 4H) LC/MS method A: R_{f}3.23 mins., (M+H)⁺==554, purity >95%.

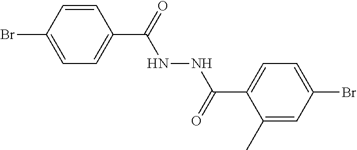 [SMILES: Cc1cc(Br)ccc1C(=O)NNC(=O)c1ccc(Br)cc1]

Synthesis of 4-Bromo-N′-(4-bromobenzoyl)-2-methylbenzohydrazide: A flask was charged with 20 mL of dichloromethane and 1460 mg (6.80 mmol) of 4-bromobenzo hydrazide and cooled in an ice water bath and 1030 mg (10.2 mmol) of triethylamine was added. Then a solution of 1620 mg (6.92 mmol) of 4-bromo-2-methylbenzoyl chloride in 4 mL of dichloromethane was added over ten minutes and stirred for 18 hours at room temp. Removed dichloromethane under vacuum and stirred with 20 mL of water and briefly sonicated. Solids isolated by decantation/filtration. Filter cake/flask rinsed with 10 mL water and 20 mL ether. Yield was 1.50 grams (53%).

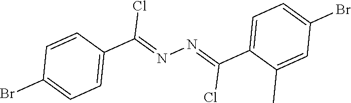 [SMILES: Cc1cc(Br)ccc1/C(Cl)=N/N=C(\Cl)c1ccc(Br)cc1]

Synthesis of (Z)-4-bromo-N-[(1Z)-(4-bromophenyl)(chloro)methylidene]-2-methyl benzene-1-carbohydrazonoyl chloride: A flask was charged with 1500 mg (3.64 mmol) of bromo-N′-(4-bromobenzoyl)-2-methylbenzohydrazide. The solid was slurried with 10 mL toluene and brief sonicated. Then 820 mg (8.74 mmol) of PCl₅ was added. The reaction was heated to 100° C. for 18 hours. The reaction was cooled and concentrated under vacuum to yield a solid. The reaction was quenched by addition of six mL of ethanol and 2 mL water. After a 30 minute age solvents removed by filtration and solid air dried for several hours. Yield 1700 mg of solid in quantitative yield.

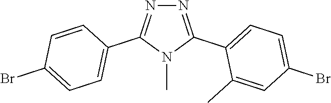 [SMILES: Cc1cc(Br)ccc1-c1nnc(-c2ccc(Br)cc2)n1C]

Synthesis of 3-(4-Bromo-2-methylphenyl)-5-(4-bromophenyl)-4-methyl-4H-1,2,4-triazole: A pressure flask was charged with (Z)-4-bromo-N-[(1Z)-(4-bromophenyl)(chloro)methylidene]-2-methylbenzene-1-carbohydrazonoyl chloride (1700 mg, 3.80 mmol) was slurried with 4 mL of dioxane and the methylamine (6.6 mL, 13.2 mmol), 2.0 M in methanol was added over two minutes. The reaction was heated to 100° C. for 18 hours and reaction cooled and solids slowly formed. Then 2 mL of water added over several minutes. Two hours later the solid was collected, air dried for 90 minutes. Yield was 1.1 grams (71%).

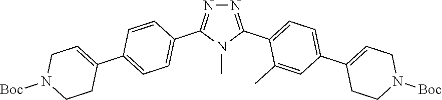 [SMILES: Cc1cc(C2=CCN(C(=O)OC(C)(C)C)CC2)ccc1-c1nnc(-c2ccc(C3=CCN(C(=O)OC(C)(C)C)CC3)cc2)n1C]

Synthesis of tert-Butyl 4-(4-(5-(4-(1-(tert-butoxycarbonyl)-1,2,3,6-tetrahydropyridin-4-yl)-2-methylphenyl)-4-methyl-4H-1,2,4-triazol-3-yl)phenyl)-5,6-dihydro pyridine-1(2H)-carboxylate: A flask was charged with 450 mg (1.11 mmol) of 3-(4-bromo-2-methylphenyl)-5-(4-bromophenyl)-4-methyl-4H-1,2,4-triazole and 4-(4,4,5,5-tetramethyl-[1,3,2]dioxaborolan-2-yl)-3,6-dihydro-2H-pyridine-1-carboxylic acid tert-butyl ester, 20 mg (0.089 mmol) of palladium acetate, 90 mg (0.22 mmol) of 2-dicyclohexyl phosphino-2′,6′-dimethoxybiphenyl, 854 mg (2.76 mmol) of tert-butyl 4 (4,4,5,5-tetra methyl-1,3,2-dioxaborolan-2-yl)-5,6-dihydropyridine-1(2H)-carboxylate, 9 mL of 1,4-dioxane and 4.4 mL of 2.0 molar potassium carbonate aqueous solution. The flask was swept with nitrogen for five minutes and heated at 95° C. for 18 hours. Then the reaction was complete by LCMS and the reaction was allowed to cool and diluted with 10 mL water and 20 mL of ethyl acetate added over two minutes. Solids formed quickly and reaction stirred for two hours. Solid was removed by filtration and ethyl acetate phase was dried and concentrated under vacuum in the hood to yield a solid which was dissolved with dichloromethane and chromatographed on silica. The column was eluted with (20% isopropanol/dichloromethane)/dichloromethane with a step gradient from 0 to 100%. Similar fractions combined and concentrated under vacuum to yield 210 mg (31. %).

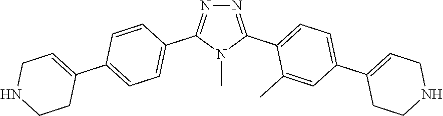 [SMILES: Cc1cc(C2=CCNCC2)ccc1-c1nnc(-c2ccc(C3=CCNCC3)cc2)n1C]

Synthesis of 4-(3-Methyl-4-(4-methyl-5-(4-(1,2,3,6-tetrahydropyridin-4-yl) phenyl)-4H-1,2,4-triazol-3-yl)phenyl)-1,2,3,6-tetrahydropyridine: A flask was charged with 210 mg (0.343 mmol) of tert-butyl 4-(4-(5-(4-(1-(tert-butoxycarbonyl)-1,2,3,6-tetrahydro pyridin-4-yl)-2-methylphenyl)-4-methyl-4H-1,2,4-triazol-3-yl)phenyl)-5,6-dihydropyridine-1(2H)-carboxylate was slurried with 3 mL dichloromethane and 4 mL (51.9 mmol) of trifluoroacetic acid added over two minutes with stirring. Two hours later the reaction was concentrated under vacuum and triturated with two 30 mL portions of ether and ether decanted away to yield a solid in quantitative yield.

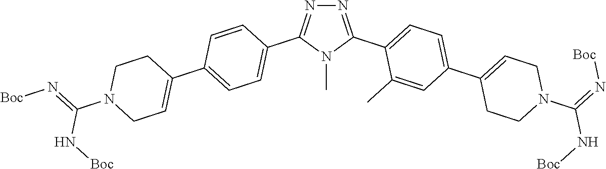 [SMILES: Cc1cc(C2=CCN(/C(=N\C(=O)OC(C)(C)C)NC(=O)OC(C)(C)C)CC2)ccc1-c1nnc(-c2ccc(C3=CCN(/C(=N/C(=O)OC(C)(C)C)NC(=O)OC(C)(C)C)CC3)cc2)n1C]

Synthesis of tert-Butyl N—[(Z)-(4-{4-[5-(4-{1-[(Z)-{[(tert-butoxy)-carbonyl]amino}({[(tert-butoxy)carbonyl]imino})methyl]-1,2,3,6-tetrahydropyridin-4-yl}-2-methyl phenyl)-4-methyl-4H-1,2,4-triazol-3-yl]phenyl}-1,2,3,6-tetrahydropyridin-1-yl)({[(tert-butoxy)carbonyl] imino})methyl]carbamate: A flask was charged with 192 mg (0.3 mmol) of 4-(3-methyl-4-(4-methyl-5-(4-(1,2,3,6-tetrahydropyridin-4-yl)phenyl)-4H-1,2,4-triazol-3-yl)phenyl)-1,2,3,6-tetrahydropyridine bis trifluoroacetic acid salt and 4 mL dichloromethane. Then 350 mg (3.47 mmol) of triethylamine added over two minutes. Then 252 mg (0.81 mmol) of tert-butyl (1H-pyrazol-1-yl)methanediylidenedicarbamate was added. The reaction slurry was stirred for 18 hours at 23° C. The reaction was concentrated under vacuum and partitioned with 30 mL ethyl acetate and 20 mL of water. Organic phase as dried and concentrated under vacuum, dissolved with dichloromethane, purified by normal phase chromatography, loaded as dichloromethane solution, eluted with (20% isopropanol/dichloromethane)/dichloromethane with step gradient from 0 to 70% to elute product. Yield was 50 mg of product (19%).

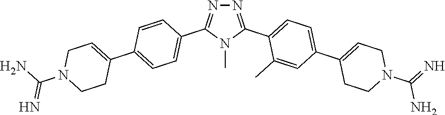 [SMILES: Cc1cc(C2=CCN(C(=N)N)CC2)ccc1-c1nnc(-c2ccc(C3=CCN(C(=N)N)CC3)cc2)n1C]

### Example 21

Synthesis of 4-(4-(5-(4-(1-Carbamimidoyl-1,2,3,6-tetrahydropyridin-4-yl)-2-methylphenyl)-4-methyl-4H-1,2,4-triazol-3-yl)phenyl)-5,6-dihydropyridine-1(2H)-carboximidamide: A flask was charged with 50 mg (0.032 mmol) of tert-butyl N—[(Z)-(4-{4-[5-(4-{1-[(Z)-{[(tert-butoxy)-carbonyl]amino}({[(tert-butoxy)carbonyl]imino})methyl]-1,2,3,6-tetrahydropyridin-4-yl}-2-methylphenyl)-4-methyl-4H-1,2,4-triazol-3-yl]phenyl}-1,2,3,6-tetrahydropyridin-1-yl)({[(tert butoxy)carbonyl]imino})methyl]carbamate was dissolved with 4 mL of dichloromethane. Then 3 mL (38.9 mmol) of trifluoroacetic acid was added over two minutes with stirring and stirred for 18 hours. The next day LCMS indicted reaction complete and reaction concentrated under vacuum. Oil was triturated with 10 mL of ether and ether decanted away to yield a solid. Solid was dissolved in N,N-dimethylformamide and was purified by prep HPLC. Yield was 16 mg (26%). ¹H NMR (300 MHz, METHANOL-d₄) δ=7.95 7.66 (m, 4H), 7.6.1-7.29 (m, 3H), 6.31 (br dd, J=4.2, 7.7 Hz, 2H), 4.33-3.98 (m, 3H), 3.96-3.67 (m, 2H), 3.66-3.45 (m, 2H), 3.28-3.14 (m, 2H), 3.07 (br d, J=1.9 Hz, 2H), 2.92-2.62 (m, 2H), 2.61-2.46 (m, 2H), 2.35-2.23 (m, 3H). LC/MS method A: R_{f}=2.65 mins., (M+H)⁺=496, purity >95%.

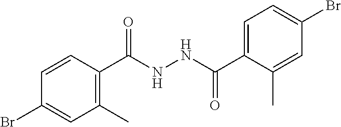 [SMILES: Cc1cc(Br)ccc1C(=O)NNC(=O)c1ccc(Br)cc1C]

Synthesis of 4-Bromo-N′-(4-bromo-2-methylbenzoyl)-2-methyl benzohydrazide: A flask was charged with 30 mL of dichloromethane and cooled in an ice water bath and 3540 mg 15.1 mmol) of 4-bromo-2-methylbenzoyl chloride was added. Then a mixture of 360 mg (7.2 mmol) of hydrazine monohydrate (2400 mg, 23.7 mmol) of triethylamine in 15 mL of dichloromethane was added over five minutes and stirred for 18 hours at 23° C. Then 15 mL of water added. After one hour of stirring solids isolated by filtration. Filter cake was rinsed with 10 mL ether. Yield was 1.42 grams (44%).

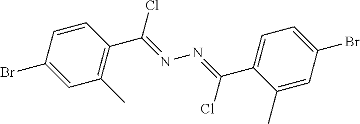 [SMILES: Cc1cc(Br)ccc1/C(Cl)=N/N=C(\Cl)c1ccc(Br)cc1C]

Synthesis of (Z)-4-Bromo-N-[(1Z)-(4-bromo-2-methylphenyl)(chloro)methylidene]-2-methylbenzene-1-carbohydrazonoyl chloride: A 250 mL flask was charged with 1420 mg (3.33 mmol) of (4-bromo-N′-(4-bromo-2-methylbenzoyl)-2-methylbenzohydrazide. The solid was slurried with 10 mL toluene and brief sonicated. 1560 mg (66.0 mmol) of PCl₅ was added in three roughly equal portions. The reaction was heated to 108° C. for 18 hours. The reaction was cooled and concentrated under vacuum to yield a solid. The reaction was quenched by addition of 10 mL ethanol and 2 mL water. After a two-hour solid collected rinsed with 10 mL of ether added and air dried for one hour. Yield 880 mg of solid (57%).

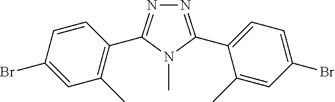 [SMILES: Cc1cc(Br)ccc1-c1nnc(-c2ccc(Br)cc2C)n1C]

Synthesis of 3,5-bis(4-Bromo-2-methylphenyl)-4-methyl-4H-1,2,4-triazole: A pressure flask was charged with (Z)-4-bromo-N-[(1Z)-(4-bromo-2-methylphenyl)(chloro) methylidene]-2-methylbenzene-1-carbohydrazonoyl chloride (880 mg, 1.90 mmol) was slurried with 3 mL of 1,4-dioxane and the methylamine (2.8 mL, 5.6 mol), 2.0 M in methanol was added over two minutes. After 30 minutes the reaction was slowly warmed to 90° C. for 18 hours. The reaction was allowed to cool and most of methanol removed under vacuum. Then 2 mL of ethyl acetate added. One hour later the solid was collected, air dried for 15 minutes and weighted 200 mg of impurities. Ethyl acetate mother liquor was diluted with 20 mL of ethyl acetate and washed with 15 mL water. Organic phase was dried and concentrated under vacuum for several hours. Yield was 680 mg. (84%).

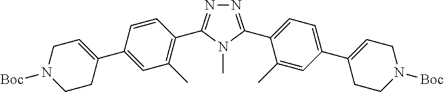 [SMILES: Cc1cc(C2=CCN(C(=O)OC(C)(C)C)CC2)ccc1-c1nnc(-c2ccc(C3=CCN(C(=O)OC(C)(C)C)CC3)cc2C)n1C]

Synthesis of tert-Butyl 4,4′-(4,4′-(4-methyl-4H-1,2,4-triazole-3,5-diyl)bis(3-methyl-4,1-phenylene))bis(5,6-dihydropyridine-1(2H)-carboxylate): A flask was charged with 300 mg (0.713 mmol) of 3,5-bis(4-bromo-2-methylphenyl)-4-methyl-4H-1,2,4-triazole, 13 mg (0.057 mmol) of palladium acetate, 59 mg (0.143 mmol) of 2-dicyclohexylphosphino-2′,6′-dimethoxybiphenyl, 572 mg (1.85 mmol) of tert-butyl 4-(4,4,5,5-tetramethyl-1,3,2-dioxaborolan-2-yl)-5,6-dihydropyridine-1(2H)-carboxylate, 4 mL of 1,4-dioxane and 2.7 mL of 2.0 molar potassium carbonate aqueous solution. The flask was swept with nitrogen for ten minutes and heated at 95° C. for 18 hours. The reaction was complete by LCMS and the reaction was allowed to cool and diluted with 10 mL water and 20 mL of ethyl acetate added over two minutes. Organic phase was dried and concentrated under vacuum and purified by silica chromatography. The column was eluted with (15% isopropanol/dichloromethane)/dichloromethane from 0 to 95% with a step gradient. Similar fractions combined and concentrated under vacuum. Yield was 280 mg (63%).

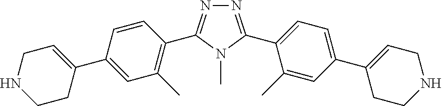 [SMILES: Cc1cc(C2=CCNCC2)ccc1-c1nnc(-c2ccc(C3=CCNCC3)cc2C)n1C]

Synthesis of 4,4′-(4,4′-(4-Methyl-4H-1,2,4-triazole-3,5-diyl)bis(3-methyl-4,1-phenylene)) bis(1,2,3,6-tetrahydropyridine): A flask was charged with 280 mg (0.447 mmol) of the from tert-butyl 4,4′-(4,4′-(4-methyl-4H-1,2,4-triazole-3,5-diyl)bis(3-methyl-4,1-phenylene))bis(5,6-dihydropyridine-1(2H)-carboxylate) which dissolved with 4 mL dichloromethane. Then 4 mL (51.9 mmol) of trifluoroacetic acid added over two minutes with stirring. Two hours later the reaction was concentrated under vacuum and triturated with 20 mL of ether and ether decanted away to yield a solid in quantitative yield.

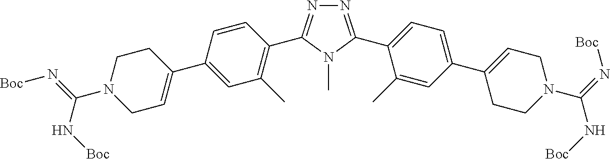 [SMILES: Cc1cc(C2=CCN(/C(=N\C(=O)OC(C)(C)C)NC(=O)OC(C)(C)C)CC2)ccc1-c1nnc(-c2ccc(C3=CCN(/C(=N/C(=O)OC(C)(C)C)NC(=O)OC(C)(C)C)CC3)cc2C)n1C]

Synthesis of tert-Butyl (4,4′-(4,4-(4-methyl-4H-1,2,4-triazole-3,5-diyl)bis(3-methyl-4,1-phenylene))bis(5,6-dihydropyridine-4,1(2H)-diyl))bis(methane-1-yl-1,1-diylidene)tetracarbamate: A flask was charged with 280 mg (0.43 mmol) of 4,4′-(4,4′-(4-methyl-4H-1,2,4-triazole-3,5-diyl)bis(3-methyl-4,1-phenylene))bis(1,2,3,6-tetrahydro pyridine)bis trifluoroacetic acid salt and 4 mL dichloromethane. Then 490 mg (4.83 mmol) of triethylamine added over two minutes followed by addition of 2 mL of N′,N-dimethyl formamide. Then 465 mg (1.50 mmol) of tert-butyl (1H-pyrazol-1-yl)methanediylidene dicarbamate was added. The reaction stirred at room temp over weekend. The reaction was concentrated under vacuum and diluted with 40 mL ethyl acetate and then washed with 20 mL of water. Organic phase was dried with sodium sulfate and concentrated under vacuum and purified by silica chromatography. The column was eluted with (15% isopropanol/dichloromethane)/dichloromethane from 0 to 95% with a step gradient. Similar fractions combined and concentrated under vacuum. Yield was 290 mg (74%).

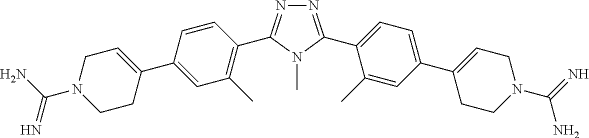 [SMILES: Cc1cc(C2=CCN(C(=N)N)CC2)ccc1-c1nnc(-c2ccc(C3=CCN(C(=N)N)CC3)cc2C)n1C]

### Example 22

Synthesis of 4,4′-(4,4′-(4-Methyl-4H-1,2,4-triazole-3,5-diyl)bis(3-methyl-4,1-phenylene))bis(5,6-dihydropyridine-1(2H)-carboximidamide): A flask was charged with 290 mg (0.320 mmol) of tert-butyl (4,4′-(4,4′-(4-methyl-4H-1,2,4-triazole-3,5-diyl)bis(3-methyl-4,1-phenylene))bis(5,6-dihydropyridine-4,1(2H)-diyl))bis(methane-1-yl-1,1-diylidene)tetracarbamate which was dissolved with 4 mL of dichloromethane. Then 4 mL (52 mmol) of trifluoroacetic acid added over two minutes with stirring and stirred for 18 hours. The next day LCMS indicted reaction complete and reaction concentrated under vacuum. Oil was triturated with two 20 mL portions of ether and ether decanted away to yield a solid. Solid was dissolved in 20% acetonitrile/water and was purified by prep HPLC. Yield was 154 mg (65%). ¹H NMR (300 MHz, CD₃OD) δ=7.59-7.44 (m, 6H), 6.44-6.18 (m, 2H), 4.28-4.08 (m, 31≥), 3.73 (t, J=5.7 Hz, 2H), 3.44-3.33 (m, 4H), 3.28-3.01 (m, 4H), 2.89-2.59 (m, 2H), 2.33 (s, 6H). LC/MS method A: R_{f}=2.71 mins., (M+H)⁺=510, purity >95%.

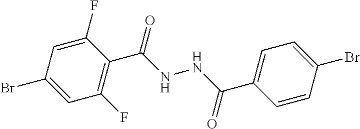 [SMILES: O=C(NNC(=O)c1c(F)cc(Br)cc1F)c1ccc(Br)cc1]

Synthesis of 4-Bromo-N-(4-bromobenzoyl)-2,6-difluorobenzohydrazide: A flask was charged with 10 mL, of N,N-dimethylformamide and charged with 1080 mg (5.02 mmol) of 4-bromobenzohydrazide and 0.1020 mg (10.1 mmol) of triethylamine and reaction cooled in an ice water bath. Then a solution of -4-bromo-2,6-difluorobenzoyl chloride in 3 mL of chloroform was added dropwise over five minutes. The reaction was allowed to stir or 18 hours at 23° C. Then 20 mL, of water was added and stirred for two hours and solids isolated by filtration. Solid dried by azeotrope with 220 mL toluene. Yield was 1800 mg (83%).

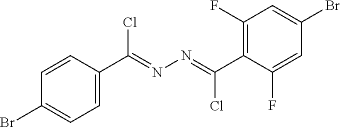 [SMILES: Fc1cc(Br)cc(F)c1/C(Cl)=N/N=C(\Cl)c1ccc(Br)cc1]

Synthesis of (Z)-4-Bromo-N-[(1Z)-(4-bromophenyl)(chloro)methylidene]-2,6-difluoro benzene-1-carbohydrazonoyl chloride: A flask was charged with 1800 mg (4.15 mmol) of 4-bromo-N′-(4-bromobenzoyl)-2,6-difluorobenzohydrazide. The solid was slurried with 15 mL of toluene and brief sonicated. 2070 mg (9.95 mmol) of PCl₅ was added. The reaction was heated to 105° C. for 18 hours. The reaction was cooled and concentrated under vacuum to yield a solid. The reaction was quenched by addition of 10 ethanol and 4 mL water. After 30 minute age solvents were removed by filtration and solid washed with 2 mL methanol and 10 mL of ether. Yield 630 mg of solid (32%).

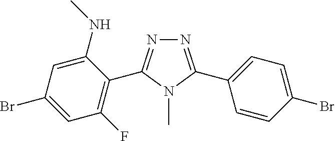 [SMILES: CNc1cc(Br)cc(F)c1-c1nnc(-c2ccc(Br)cc2)n1C]

Synthesis of 5-Bromo-2-(5-(4-bromophenyl)-4-methyl-4H-1,2,4-triazol-3-yl)-3-fluoro-N-methylaniline: A pressure flask was charged with the crude (Z)-4-bromo-N-[(1Z)-(4-bromophenyl)(chloro)methylidene]-2,6-difluorobenzene-1-carbohydrazonoyl chloride (919 mg, 0.1.93 mmol) was slurried with 3 mL of 1,4-dioxane and the methylamine (4 mL, 8 mmol), 2.0 M in methanol was added over two minutes. The reaction was heated at 105° C. for 18 hours. The reaction was allowed to cool and 2 mL of water added. One hour later the solid was collected, rinsed with 0.10 mL water and several mL of ether and air dried. Yield was 503 mg (43%).

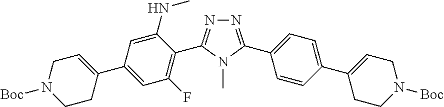 [SMILES: CNc1cc(C2=CCN(C(=O)OC(C)(C)C)CC2)cc(F)c1-c1nnc(-c2ccc(C3=CCN(C(=O)OC(C)(C)C)CC3)cc2)n1C]

Synthesis of tert-Butyl 4-(4-(5-(4-(1-(tert-butoxycarbonyl)-1,2,3,6-tetrahydro pyridin-4-yl)-2-fluoro-6-(methylamino)phenyl)-4-methyl-4H-1,2,4-triazol-3-yl)phenyl)-5,6-dihydro pyridine-1(2H)-carboxylate: A flask was charged with 502 mg (1.14 mmol) of 5-bromo-2-(5-(4-bromophenyl)-4-methyl-4H-1,2,4-triazol-3-yl)-3-fluoro-N-methylaniline, 25 mg (0.11 mmol) of palladium acetate, 117 mg (0.11 mmol) of 2-dicyclohexylphosphino-2′,6′-dimethoxybiphenyl, 986 mg (3.19 mmol) of tert-butyl 4-(4,4,5,5-tetramethyl-1,3,2-dioxaborolan-2-yl)-5,6-dihydropyridine-1(2H)-carboxylate, 9 mL of dioxane and 4.2 mL of 2.0 molar potassium carbonate aqueous solution. The flask was swept with nitrogen for five minutes and heated at 95° C. for 18 hours. Then the reaction was complete by LCMS and the reaction was allowed to cool and diluted with 10 water and 30 mL of ethyl acetate added over two minutes. Organic phase dried and concentrated under vacuum and chromatographed on silica with (20% isopropanol/dichloromethane)/dichloromethane 0 to 70% step gradient used to elute product. Yield 777 mg in a quantitative yield.

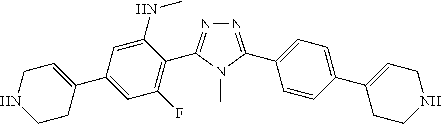 [SMILES: CNc1cc(C2=CCNCC2)cc(F)c1-c1nnc(-c2ccc(C3=CCNCC3)cc2)n1C]

Synthesis of 3-Fluoro-N-methyl-2-(4-methyl-5-(4-(1,2,3,6-tetrahydropyridin-4-yl)phenyl)-4H-1,2,4-triazol-3-yl)-5-(1,2,3,6-tetrahydropyridin-4-yl)aniline: The tert-butyl 4-(4-(5-(4-(1-(tert-butoxycarbonyl)-1,2,3,6-tetrahydropyridin-4-yl)-2-fluoro-6-(methyl amino)phenyl)-4-methyl-4H-1,2,4-triazol-3-yl)phenyl)-5,6-dihydropyridine (310 mg, 0.481 mmol) was dissolved with 5 mL dichloromethane. Then 8 mL (104 mmol) of trifluoroacetic acid added over five minutes with stirring. Three hours later the reaction was concentrated under vacuum and triturated with two 20 mL portions of ether and ether decanted away to yield a solid in quantitative yield.

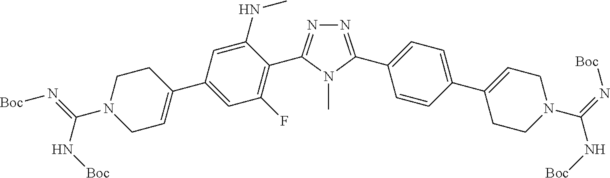 [SMILES: CNc1cc(C2=CCN(/C(=N/C(=O)OC(C)(C)C)NC(=O)OC(C)(C)C)CC2)cc(F)c1-c1nnc(-c2ccc(C3=CCN(/C(=N\C(=O)OC(C)(C)C)NC(=O)OC(C)(C)C)CC3)cc2)n1C]

Synthesis of tert-Butyl N-[(E)-(4-{4-[5-(4-{1-[(E)-{[(tert-butoxy)-carbonyl]amino}({[(tert-butoxy)carbonyl]imino})methyl]-1,2,3,6-tetrahydropyridin-4-yl}phenyl)-4-methyl-4H-1,2,4-triazol-3-yl]-3-fluoro-5-(methylamino)phenyl}-1,2,3,6-tetrahydropyridin-1-yl)({[(tert-butoxy) carbonyl]imino})methyl]carbamate: A flask was charged with 310 mg (0.46 mmol) of 3-fluoro-N-methyl-2-(4-methyl-5-(4-(1,2,3,6-tetrahydropyridin-4-yl)phenyl)-4H-1,2,4-triazol-3-yl)-5-(1,2,3,6-tetrahydropyridin-4-yl) aniline bis trifluoroacetic acid salt and 5 mL dichloromethane. Then 980 mg (9.70 mmol) of triethylamine added over two minutes. Then 442 mg (1.43 mmol) of tert-butyl (1H-pyrazol-1-yl)methanediylidenedicarbamate was added. The reaction was stirred for 18 hours at 23° C. Then the reaction was concentrated under vacuum and concentrate was diluted with 50 mL ethyl acetate and then washed with 30 mL of water. Organic phase was dried with sodium sulfate and concentrated under vacuum. The product was purified by normal phase chromatography, loaded as dichloromethane solution, eluted with (20% isopropanol/dichloromethane)/dichloromethane with step gradient from 0 to 100% to elute product. Yield was 170 mg of product (40%).

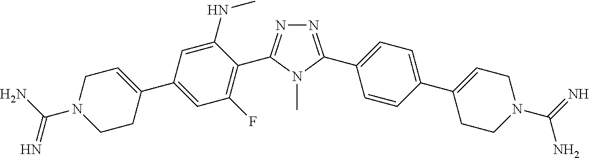 [SMILES: CNc1cc(C2=CCN(C(=N)N)CC2)cc(F)c1-c1nnc(-c2ccc(C3=CCN(C(=N)N)CC3)cc2)n1C]

### Example 23

Synthesis of 4-(4-(5-(4-(1-Carbamimidoyl-1,2,3,6-tetrahydropyridin-4-yl)-2-fluoro-6-(methyl amino)phenyl)-4-methyl-4H-1,2,4-triazol-3-yl)phenyl)-5,6-dihydropyridine-1(2H)-carboximidamide: A flask was charged with 170 mg (0.184 mmol) of tert-butyl N-[(E)-(4-{4-[5-(4-{1-[(E)-{[(tert-butoxy)carbonyl]amino}({[(tert-butoxy)carbonyl]imino})methyl]-1,2,3,6-tetra hydropyridin-4-yl}phenyl)-4-methyl-4H-1,2,4-triazol-3-yl]-3-fluoro-5-(methylamino)phenyl}-1,2,3,6-tetrahydropyridin-1-yl)({[(tert-butoxy)carbonyl]imino})methyl]carbamate which was dissolved with 4 mL of dichloromethane. Then 4 (51.9 mmol) of trifluoroacetic acid was added over two minutes with stirring and stirred for two hours. LCMS indicted reaction complete and reaction concentrated under vacuum. Oil was triturated with two 30 mL portions of ether and ether decanted away to yield a solid. Solid was dissolved in 20% aqueous N,N-dimethylformamide and was purified by prep HPLC. Yield was 32 mg (23%). ¹H NMR (300 MHz, METHANOL-d₄) δ=7.88-7.68 (m, 2H), 7.32 (s, 2H), 6.71-6.59 (m, 2H), 6.47-6.14 (m, 2H), 4.23-4.13 (m, 3H), 3.98 (s, 3H), 3.87-3.64 (m, 2H), 3.63-3.46 (m, 2H), 3.27-3.03 (m, 2H), 2.91-2.52 (m, 2H), 2.37 (m, 2H). LC/MS method A: R_{f}=2.85 mins., (M+H)⁺=529, purity >95%.

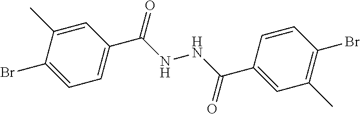 [SMILES: Cc1cc(C(=O)NNC(=O)c2ccc(Br)c(C)c2)ccc1Br]

Synthesis of 4-Bromo-N′-(4-bromo-3-methylbenzoyl)-3-methyl benzohydrazide: A flask was charged with 2630 mg (11.3 mmol) of 4-bromo-3-methyl benzoyl chloride and 35 mL chloroform. The solution was cooled in an ice/water bath and a mixture of 5 mL chloroform, hydrazine hydrate (271 mg, 5.42 mmol), N,N-diisopropylethyl amine (1900 mg, 14.7 mmol) was added dropwise over five minutes. The reaction was allowed to slowly warm to 23° C. and stirred for 18 hours. Then the chloroform was removed under vacuum and the concentrate was sonicated for five minutes with 20 mL of water. After a two-hour age the solid was collected and slurried with 70 mL acetonitrile and solid collected and air dried for 18 hours to yield 2.4 grams (99% yield).

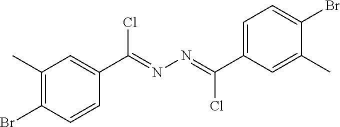

Synthesis of (Z)-4-Bromo-N-[(1Z)-(4-bromo-3-methylphenyl)(chloro) methylidene]-3-methylbenzene-1-carbohydrazonoyl chloride: A flask was charged with 2400 mg (5.66 mmol) of 4-Bromo-N′-(4-bromo-3-methylbenzoyl)-3-methyl benzohydrazide. The solid was slurried with 20 mL of toluene and briefly sonicated. Then 5000 mg (24.0 mmol) of PCl₅ was added at once. The reaction was heated to 100° C. for 18 hours. The reaction was cooled and concentrated under vacuum to yield a solid. The reaction was quenched by addition of wet ice (about 25 grams ice) and 10 mL of water After a two-hour age water removed by decantation and solid rinsed with 10 mL methanol and air dried in the hood for an hour. Yield 3.2 grams of solid (88%).

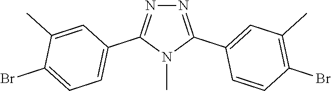 [SMILES: Cc1cc(-c2nnc(-c3ccc(Br)c(C)c3)n2C)ccc1Br]

Synthesis of 3,5-Bis(4-bromo-3-methylphenyl)-4-methyl-4H-1,2,4-triazole: A flask was charged with the crude (Z)-4-bromo-N-[(1Z)-(4-bromo-3-methylphenyl)(chloro)methylidene]-3-methylbenzene-1-carbohydrazonoyl chloride (3.2 grams, 7.0 mmol) was slurried with 20 mL of methanol and 1740 mg (25.7 mmol) of methylamine hydrochloride was added at once. Then 4060 mg (31.5 mmol) of diisopropylethylamine was added over ten minutes. After 30 minutes the reaction was slowly warmed to 65° C. and heated for 18 hours with a septum open to air via syringe needle. The reaction was cooled, and 10 mL of water added over ten minutes. The reaction was heated at 80° C. for three hours. The reaction was allowed to cool to 23° C., then water (10 mL) was added. The mixture was allowed to stir for one hour and solid collected and air dried in hood for one hour to yield 410 mg (13%).

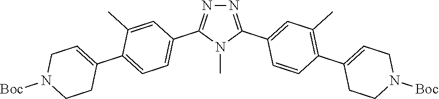 [SMILES: Cc1cc(-c2nnc(-c3ccc(C4=CCN(C(=O)OC(C)(C)C)CC4)c(C)c3)n2C)ccc1C1=CCN(C(=O)OC(C)(C)C)CC1]

Synthesis of tert-Butyl 4,4′-(4,4′-(4-methyl-4H-1,2,4-triazole-3,5-diyl)bis(2-methyl-4,1-phenylene))bis(5,6-dihydropyridine-1(2H)-carboxylate): A flask was charged with 400 mg (0.95 mmol) of 3,5-bis(4-bromo-3-methylphenyl)-4-methyl-4H-1,2,4-triazole, 17 mg (0.076 mmol) of palladium acetate, 78 mg (0.19 mmol) of 2-dicyclohexylphosphino-2′,6′-dimethoxybiphenyl, 736 mg (2.38 mmol) of tert-butyl 4-(4,4,5,5-tetramethyl-1,3,2-dioxaborolan-2-yl)-5,6-dihydropyridine-1(2H)-carboxylate, 6 mL of 1,4-dioxane and 3.7 mL of 2.0 molar potassium carbonate aqueous solution. The flask was swept with nitrogen for five minutes and heated at 95° C. for 18 hours. The reaction was complete by LCMS and the reaction was allowed to cool and diluted with 5 mL water and 4 mL of ethyl acetate added over two minutes. Solids formed quickly and reaction stirred for 90 minutes. Solid collected on a filter and rinsed with 3 mL ethyl acetate and air-dried for two hours in the hood to yield 250 mg (42%).

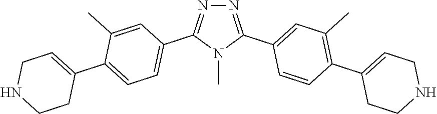 [SMILES: Cc1cc(-c2nnc(-c3ccc(C4=CCNCC4)c(C)c3)n2C)ccc1C1=CCNCC1]

Synthesis of 4,4′-(4,4′-(4-Methyl-4H-1,2,4-triazole-3,5-diyl)bis(2-methyl-4,1-phenylene))bis(1,2,3,6-tetrahydropyridine): A flask was charged with 250 mg (0.40 mmol) of tert-butyl4,4′-(4,4′-(4-methyl-4H-1,2,4-triazole-3,5-diyl)bis(2-methyl-4,1-phenylene))bis(5,6-dihydro pyridine-1(2H)-carboxylate) was slurried with 500 mg (4.63 mmol) of anisole and 3 mL dichloromethane. Then 5 mL (64.5 mmol) of trifluoroacetic acid added over three minutes with stirring. After stirring for 18 hours at 23° C., the reaction was concentrated under vacuum and triturated with two 30 mL portions of ether and ether decanted away to yield a solid in 100% yield.

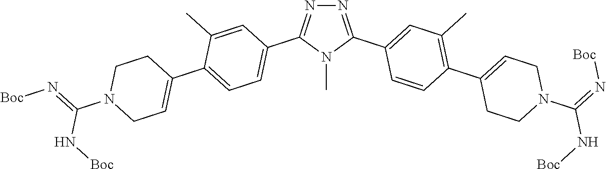 [SMILES: Cc1cc(-c2nnc(-c3ccc(C4=CCN(/C(=N/C(=O)OC(C)(C)C)NC(=O)OC(C)(C)C)CC4)c(C)c3)n2C)ccc1C1=CCN(/C(=N\C(=O)OC(C)(C)C)NC(=O)OC(C)(C)C)CC1]

Synthesis of tert-Butyl (4,4′-(4,4′-(4-methyl-4H-1,2,4-triazole-3,5-diyl)bis(2-methyl-4,1-phenylene))bis(5,6-dihydropyridine-4,1(2H)-diyl))bis(methane-1-yl-1,1-diylidene) tetracarbamate: A flask was charged with 260 mg (0.40 mmol) of 4,4′-(4,4′-(4-methyl-4H-1,2,4-triazole-3,5-diyl)bis(2-methyl-4,1-phenylene))bis(1,2,3,6-tetrahydro pyridine) bis trifluoroacetic acid salt and 3 mL N,N-dimethylformamide. Then 424 mg (4.2 mmol) of triethylamine added over five minutes. Then 477 mg (1.54 mmol) of tert-butyl (1H-pyrazol-1-yl)methanediylidenedicarbamate was added at once. After stirring for 18 hours at 23° C., the reaction was diluted with 30 mL ethyl acetate and then washed with three portions of 20 mL of water. Organic phase was dried with sodium sulfate and concentrated under vacuum to yield 410 mg of crude production in quantitative yield.

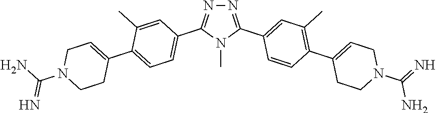 [SMILES: Cc1cc(-c2nnc(-c3ccc(C4=CCN(C(=N)N)CC4)c(C)c3)n2C)ccc1C1=CCN(C(=N)N)CC1]

### Example 24

Synthesis of 4,4′-(4,4′-(4-Methyl-4H-1,2,4-triazole-3,5-diyl)bis(2-methyl-4,1-phenylene)) bis(5,6-dihydropyridine-1(2H)-carboximidamide): A flask was charged with 410 mg (0.400 mmol) of from tert-butyl (4,4-(4,4-(4-methyl-4H-1,2,4-triazole-3,5-diyl)bis(2-methyl-4,1-phenylene))bis(5,6-dihydropyridine-4,1(2H)-diyl))bis(methane-1-yl-1,1-diylidene)tetracarbamate was dissolved with a mixture of 800 mg (7.41 mmol) of anisole and 4 mL, of dichloromethane. Then 7 mL (90.9 mmol) of trifluoroacetic acid was added over five minutes with stirring and stirred for 18 hours. The next day LCMS indicted reaction complete and reaction concentrated under vacuum. Oil was dissolved with N,N-dimethylformamide and was purified by prep HPLC. Yield was 143 mg (48%). ¹H NMR (300 MHz, CD₃OD) δ=7.78-7.50 (m, 3H), 7.39 (br d, J=8.0 Hz, 3H), 5.76 (s, 2H), 4.14 (s, 3H), 3.80 (m, 2H), 3.73 (m, 2H), 2.57 (m, 4H), 2.44 (m, 2H), 2.15 (d, J=3.0 Hz, 6H). LC/MS method A: R_{f}=2.53 mins., (M+H)⁺=510, purity >95%.

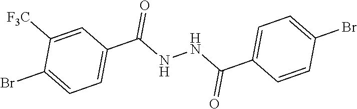 [SMILES: O=C(NNC(=O)c1ccc(Br)c(C(F)(F)F)c1)c1ccc(Br)cc1]

Synthesis of 4-Bromo-N′-(4-bromobenzoyl)-3-(trifluoromethyl) benzohydrazide: A flask was charged with 2100 mg (9.86 mmol) of 4-bromobenzohydrazide and slurried with 20 mL of chloroform and 1720 mg (13.3 mmol) of N,N-diisopropylethyl amine and cooled in an ice water bath. Then a solution of 4-bromo-3-(trifluoromethyl) benzoyl chloride. (2960 mg, 10.3 mmol) in 20 mL chloroform was added dropwise over 30 minutes. The reaction was allowed to stir for 18 hours at 23° C. The reaction was concentrated under vacuum and briefly sonicated with 25 mL of water and aged for one hour. Water decanted away and 40 mL acetonitrile added and removed under vacuum to dry solid. Yield was 4.400 nag (97%).

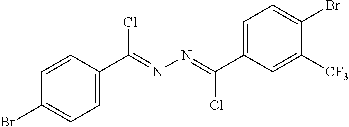 [SMILES: FC(F)(F)c1cc(/C(Cl)=N/N=C(\Cl)c2ccc(Br)cc2)ccc1Br]

Synthesis of (Z)-4-bromo-N-[(1Z)-(4-bromophenyl)(chloro)methylidene]-3-(trifluoromethyl)benzene-1-carbohydrazonoyl chloride: A flask was charged with 44430 mg (9.50 mmol of 4-bromo-N′-(4-bromobenzoyl)-3-(trifluoromethyl) and slurried with 30 mL toluene. Then 5930 mg (28.6 mmol) of phosphorus pentachloride was added and the reaction was heated to 105° C. for 18 hours. The reaction was allowed to cool and concentrated under vacuum. The concentrate was quenched by addition of 25 grams of ice. After 90 minutes the solid was collected and rinsed with 15 mL of methanol. Then 40 mL acetonitrile added and removed under vacuum. Yield was 4490 mg (95%).

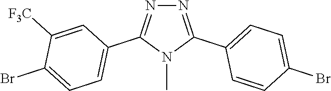 [SMILES: Cn1c(-c2ccc(Br)cc2)nnc1-c1ccc(Br)c(C(F)(F)F)c1]

Synthesis of 3-(4-Bromo-3-(trifluoromethyl)phenyl)-5-(4-bromophenyl)-4-methyl-4H-1,2,4-triazole: A flask was charged with 4500 mg (9.0 mmol) of (Z)-4-bromo-N-[(1Z)-(4-bromophenyl)(chloro)methylidene]-3-(trifluoromethyl)benzene-1-carbohydrazonoyl chloride and slurried with 20 mL of methanol. Then 1820 mg (27.9 mmol) of methylamine hydrochloride was added, followed by addition of 4060 mg (31.5 mmL) of N,N-diisopropylethylamine added over five minutes. Then the reaction was heated to 65° C. for 18 hours with a needle in septum to allow excess methylamine to escape. The reaction was cooled and diluted with 30 mL of water. After 30 minutes solid collected, rinsed with five mL water and air dried for one hour. Then 50 mL acetonitrile added and removed under vacuum to yield 2100 mg of product (51% yield).

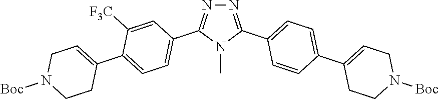 [SMILES: Cn1c(-c2ccc(C3=CCN(C(=O)OC(C)(C)C)CC3)cc2)nnc1-c1ccc(C2=CCN(C(=O)OC(C)(C)C)CC2)c(C(F)(F)F)c1]

Synthesis of tert-Butyl 4-(4-(5-(4-(1-(tert-butoxycarbonyl)-1,2,3,6-tetrahydro pyridin-4-yl)-3-(trifluoromethyl)phenyl)-4-methyl-4H-1,2,4-triazol-3-yl)phenyl)-5,6-dihydro pyridine-1(2H)-carboxylate: A flask was charged with 207 mg (0.45 mmol) of 3-(4-bromo-3-(trifluoromethyl)phenyl)-5-(4-bromophenyl)-4-methyl-4H-1,2,4-triazole, 335 mg (1.08 mmol) of tert-butyl 4-(4,4,5,5-tetramethyl-1,3,2-dioxaborolan-2-yl)-5,6-dihydropyridine-1(2H)-carboxylate, 8 mg (0.036 mmol of palladium acetate, and 37 mg (0.09 mmol) of 2-dicyclohexylphosphino-2′,6′-dimethoxybiphenyl, 5 mL of 1,4-dioxane and 1.7 mL of 2.0 molar potassium carbonate in water. After heating for 18 hours at 95° C. The reaction was allowed to cool and diluted with 15 mL water and 50 mL ethyl acetate. Organic phase was dried with sodium sulfate and concentrated under vacuum. Yield was 320 mg (quantitative yield) and directly reacted.

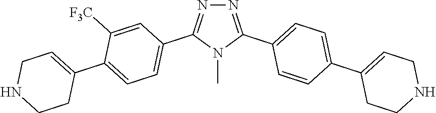 [SMILES: Cn1c(-c2ccc(C3=CCNCC3)cc2)nnc1-c1ccc(C2=CCNCC2)c(C(F)(F)F)c1]

Synthesis of 4-(4-(4-Methyl-5-(4-(1,2,3,6-tetrahydropyridin-4-yl)-3-(trifluoromethyl) phenyl)-4H-1,2,4-triazol-3-yl)phenyl)-1,2,3,6-tetrahydropyridine: A flask was charged with 320 mg (0.45 mmol) of tert-butyl 4-(4-(5-(4-(1-(tert-butoxycarbonyl)-1,2,3,6-tetrahydropyridin-4-yl)-3-(trifluoromethyl)phenyl)-4-methyl-4H-1,2,4-triazol-3-yl)phenyl)-5,6-dihydropyridine-1(2H)-carboxylate. Then 5 mL dichloromethane and 0.5 mL (4.63 mmol) of anisole was added to dissolve material and 12 mL (157 mmol) of trifluoroacetic acid was added over several minutes. After several hours dichloromethane removed under vacuum and 6 mL (79 mmol) of trifluoroacetic acid was added over several minutes. One hour later the reaction was concentrated under vacuum and triturated with two 30 mL portions of ether. Yield was quantitative.

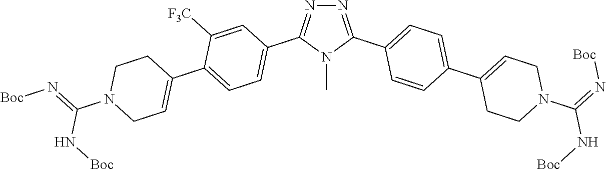 [SMILES: Cn1c(-c2ccc(C3=CCN(/C(=N\C(=O)OC(C)(C)C)NC(=O)OC(C)(C)C)CC3)cc2)nnc1-c1ccc(C2=CCN(/C(=N/C(=O)OC(C)(C)C)NC(=O)OC(C)(C)C)CC2)c(C(F)(F)F)c1]

Synthesis of tert-Butyl N—[(Z)-(4-{4-[5-(4-{1-[(Z)-{[(tert-butoxy)carbonyl]amino}-({[(tert-butoxy)carbonyl]imino})methyl]-1,2,3,6-tetrahydropyridin-4-yl}-3-(trifluoro methyl)phenyl)-4-methyl-4H-1,2,4-triazol-3-yl]phenyl}-1,2,3,6-tetrahydropyridin-1-yl)({[(tert-butoxy)carbonyl] imino})methyl]carbamate: A flask as charged with 700 mg of (1.500 mmol) of 4-(4-(4-methyl-5-(4-(1,2,3,6-tetrahydropyridin-4-yl)-3-(trifluoromethyl)phenyl)-4H-1,2,4-triazol-3-yl)phenyl)-1,2,3,6-tetrahydropyridine bis trifluoroacetic acid salt as a solution in 7 mL N,N-dimethylformamide. Then 909 mg (9.0 mmol) of triethylamine was added over several minutes. Five minutes later 100 mg (3.56 mmol) of tert-butyl (1H-pyrazol-1-yl)methanediylidenedicarbamate was added at once. After stirring for 18 hours an additional 300 mg (0.968 mmol) of tert-butyl(1H-pyrazol-1-yl)methanediylidenedicarbamate was added at once. After 0.18 hours, the reaction was diluted with 50 mL ethyl acetate and then washed with three portions of 30 mL of water. Organic phase was dried with sodium sulfate and concentrated under vacuum. Yield was 1750 mg of crude material in a quantitative yield.

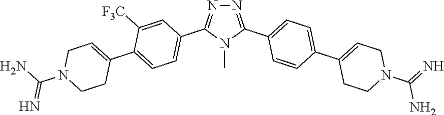 [SMILES: Cn1c(-c2ccc(C3=CCN(C(=N)N)CC3)cc2)nnc1-c1ccc(C2=CCN(C(=N)N)CC2)c(C(F)(F)F)c1]

### Example 25

Synthesis of 4-(4-(5-(4-(1-Carbamimidoyl-1,2,3,6-tetrahydropyridin-4-yl)-3-(trifluoromethyl)phenyl)-4-methyl-4H-1,2,4-triazol-3-yl)phenyl)-5,6-dihydropyridine-1(2H)-carboximidamide: A flask was charged with 1750 mg (1.50 mmol) and dissolved by addition of 5 mL of dichloromethane, 800 mg (7.4 mmol) of anisole and 7 mL (91 mmol) of trifluoroacetic acid. After two hours, the reaction was concentrated under vacuum, dissolved with N,N-dimethylformamide and purified by reverse phase prep HPLC. Similar fractions were combined and freeze dried. Yield was 310 mg (27%). ¹H NMR (300 MHz, CD₃OD) δ=8.17 (d, J=1.7 Hz, 1H), 8.05 (dd, J=1.7, 8.1 Hz, 1H), 7.83-7.61 (m, 3H), 7.48-7.21 (m, 2H), 6.38-6.27 (m, 1H), 5.79 (br t, J=3.0 Hz, 1H), 4.26-4.03 (m, 3H), 3.80 (s, 2H), 3.85-3.65 (m, 2H), 3.54-3.33 (m, 2H), 2.77 (br dd, J=2.5, 4.2 Hz, 2H), 2.64-2.56 (m, 2H). LC/MS method A: R_{f}=2.76 mins., (M+H)⁺=550, purity >95%.

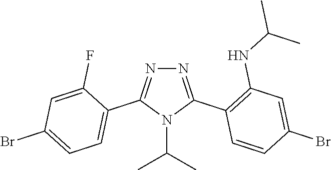 [SMILES: CC(C)Nc1cc(Br)ccc1-c1nnc(-c2ccc(Br)cc2F)n1C(C)C]

5-Bromo-2-(5-(4-bromo-2-fluorophenyl)-4-isopropyl-4H-1,2,4-triazol-3-yl)-N-isopropylaniline A screw topped vial was charged with (Z)-4-bromo-N-[(1Z)-(4-bromo-2-fluorophenyl)(chloro)methylidene]-2-fluorobenzene-1-carbohydrazonoyl chloride (770 mg, 1.63 mmol) was slurried with 3 mL of methanol and the isopropylamine (192 mg, 3.26 mol), was added at once. Five minutes later 420 mg (3.26 mmol) of diisopropylethylamine was added over one minute. After five minutes the reaction was warmed to 55° C. and stirred for 18 hours. The reaction was cooled and most of methanol removed under vacuum. Then 4 mL of N,N-dimethylformamide was added and reaction heated to 111. ° C. for 18 hours, cooled, diluted with 30 mL ethyl acetate, washed with three 20 mL portions of water, dried and concentrated under vacuum. Yield was 170 mg (21%).

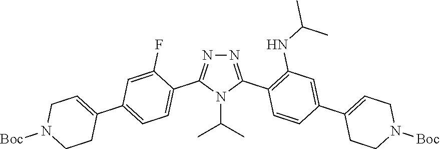 [SMILES: CC(C)Nc1cc(C2=CCN(C(=O)OC(C)(C)C)CC2)ccc1-c1nnc(-c2ccc(C3=CCN(C(=O)OC(C)(C)C)CC3)cc2F)n1C(C)C]

Synthesis of tert-Butyl 4-(4-(5-(4-(1-(tert-butoxycarbonyl)-1,2,3,6-tetrahydropyridin-4-yl)-2-(isopropylamino)phenyl)-4-isopropyl-4H-1,2,4-triazol-3-yl)-3-fluorophenyl)-5,6-dihydro pyridine-1(2H)-carboxylate A flask was charged with 170 mg (0.342 mmol) of 5-bromo-2-(5-(4-bromo-2-fluorophenyl)-4-isopropyl-4H-1,2,4-triazol-3-yl)-N-isopropylaniline and tert-butyl 4-(4,4,5,5-tetramethyl-1,3,2-dioxaborolan-2-yl)-5,6-dihydropyridine-1(2H)-carboxylate. 6.1 mg (0.027 mmol) of palladium acetate, 28 mg (0.068 mmol) of 2-dicyclohexylphosphino-2′,6′-dimethoxybiphenyl, 265 mg (0.855 mmol) of tert-butyl 4-(4,4,5,5-tetramethyl-1,3,2-dioxaborolan-2-yl)-5,6-dihydropyridine-1(2H)-carboxylate, 4 mL of 1,4-dioxane and 1.3 mL of 2.0 molar potassium carbonate aqueous solution. The flask was swept with nitrogen for five minutes and heated at 95° C. for 18 hours. The reaction was complete by LCMS and the reaction was allowed to cool and diluted with 20 mL water and 30 mL of ethyl acetate added over two minutes. The organic phase was dried and concentrated under vacuum to yield 300 mg, quantitative yield of crude product (70%).

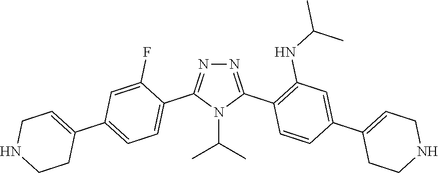 [SMILES: CC(C)Nc1cc(C2=CCNCC2)ccc1-c1nnc(-c2ccc(C3=CCNCC3)cc2F)n1C(C)C]

Synthesis of 2-(5-(2-Fluoro-4-(1,2,3,6-tetrahydropyridin-4-yl)phenyl)-4-isopropyl-4H-1,2,4-triazol-3-yl)-N-isopropyl-5-(1,2,3,6-tetrahydropyridin-4-yl)aniline: A flask was charged with 240 mg (0.342 mmol) of tert-butyl 4-(4-(5-(4-(1-(tert-butoxy carbonyl)-1,2,3,6-tetrahydropyridin-4-yl)-2-(isopropylamino)phenyl)-4-isopropyl-4H-1,2,4-triazol-3-yl)-3-fluoro phenyl)-5,6-dihydropyridine-1(2H)-carboxylate was dissolved with 5 mL dichloromethane. Then 7 mL (90.0 mmol) of trifluoroacetic acid added over five minutes with stirring. Then two hours later the reaction was concentrated under vacuum and triturated with two 20 mL portions of ether and ether decanted away to yield a solid in quantitative yield.

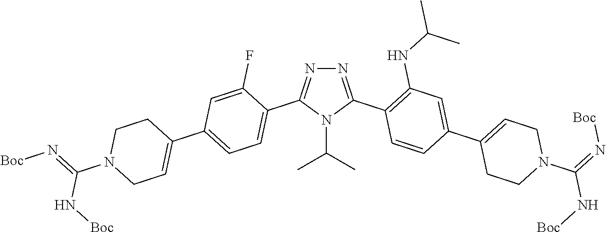 [SMILES: CC(C)Nc1cc(C2=CCN(/C(=N\C(=O)OC(C)(C)C)NC(=O)OC(C)(C)C)CC2)ccc1-c1nnc(-c2ccc(C3=CCN(/C(=N/C(=O)OC(C)(C)C)NC(=O)OC(C)(C)C)CC3)cc2F)n1C(C)C]

Synthesis of tert-Butyl N-({4-[4-(5-{4-[1-({[(tert-butoxy)carbonyl]-amino}({[(tert-butoxy) carbonyl]imino})methyl)-1,2,3,6-tetrahydropyridin-4-yl]-2-[(propan-2-yl)amino]phenyl}-4-(propan-2-yl)-4H-1,2,4-triazol-3-yl)-3-fluorophenyl]-1,2,3,6-tetrahydropyridin-1-yl}({[(tert-butoxy) carbonyl]imino})methyl)carbamate: A flask was charged with 250 mg (0.342 mmol) of 2-(5-(2-fluoro-4-(1,2,3,6-tetrahydropyridin-4-yl)phenyl)-4-isopropyl-4H-1,2,4-triazol-3-yl)-N-isopropyl-5-(1,2,3,6-tetrahydropyridin-4-yl)aniline bis trifluoroacetic acid salt and 4 mL N,N-dimethylformamide. Then 230 mg (2.27 mmol) of triethylamine was added at once. Then 223 mg (0.72 mmol) of tert-butyl (1H-pyrazol-1-yl)methanediylidenedicarbamate was added. The reaction was stirred for 18 hours at 23° C. The reaction was diluted with 30 mL ethyl acetate and then washed with three portions of 20 mL of water. Organic phase was dried with sodium sulfate and concentrated under vacuum to yield the crude product in quantitative yield (370 mg).

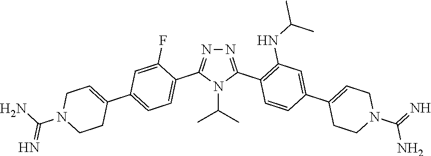 [SMILES: CC(C)Nc1cc(C2=CCN(C(=N)N)CC2)ccc1-c1nnc(-c2ccc(C3=CCN(C(=N)N)CC3)cc2F)n1C(C)C]

### Example 26

Synthesis of 4-(4-(5-(4-(1-Carbamimidoyl-1,2,3,6-tetrahydropyridin-4-yl)-2-(isopropylamino)phenyl)-4-isopropyl-4H-1,2,4-triazol-3-yl)-3-fluorophenyl)-5,6-dihydro pyridine-1(2H)-carboximidamide: A flask was charged with 370 mg (0.342 mmol) of tert-butyl N-({4-[4-(5-{4-[1-({[(tert-butoxy)carbonyl]amino}({[(tert-butoxy)carbonyl]imino})methyl)-1,2,3,6-tetrahydropyridin-4-yl]-2-[(propan-2-yl)amino]phenyl}-4-(propan-2-yl)-4H-1,2,4-triazol-3-yl)-3-fluorophenyl]-1,2,3,6-tetrahydropyridin-1-yl}({[(tert-butoxy)carbonyl]imino})methyl)carbamate was dissolved with a mixture of 300 mg (2.78 mmol) of anisole and 3 mL of dichloromethane. Then 5 mL (64.9 mmol) of trifluoroacetic acid added over two minutes with stirring and stirred for 18 hours. The next day LCMS indicted reaction complete and reaction concentrated under vacuum. Oil was triturated with two 30 mL portions of ether and ether decanted away to yield a solid. Solid was dissolved in 7 mL N,N-dimethylformamide and was purified by prep HPLC. Yield was 42 mg (13%). ¹H NMR (300 MHz, METHANOL-d₄) δ=7.79-7.62 (m, 1H), 7.6.1-7.47 (m, 2H), 7.37 (br s, 2H), 6.53-6.27 (m, 2H), 4.88-4.76 (m, 2H), 4.29-4.08 (m, 2H), 3.74 (t, J==5.7 Hz, 4H), 3.59-3.32 (m, 4H), 2.75 (br dd, J==4.1, 6.6 Hz, 12H). LC/MS method A: R_{f}=3.07 mins., (M+H)⁺=586, purity >95%.

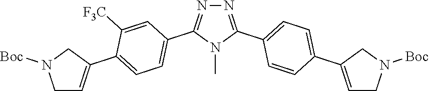 [SMILES: Cn1c(-c2ccc(C3=CCN(C(=O)OC(C)(C)C)C3)cc2)nnc1-c1ccc(C2=CCN(C(=O)OC(C)(C)C)C2)c(C(F)(F)F)c1]

Synthesis of tert-Butyl 3-(4-(5-(4-(1-(tert-butoxycarbonyl)-2,5-dihydro-1H-pyrrol-3-yl)-3-(trifluoromethyl)phenyl)-4-methyl-4H-1,2,4-triazol-3-yl)phenyl)-2,5-dihydro-1H-pyrrole-1-carboxylate: A flask was charged with 432 mg (0.95 mmol) of 3-(4-bromo-3-(trifluoromethyl)phenyl)-5-(4-bromophenyl)-4-methyl-4H-1,2,4-triazole, 14 mg (0.064 mmol) of palladium acetate, 66 mg (0.16 mmol) of 2-dicyclohexylphosphino-2′,6′-dimethoxybiphenyl, 590 mg (2.00 mmol) of tert-butyl 3-(4,4,5,5-tetramethyl-1,3,2-dioxaborolan-2-yl)-2,5-dihydro-1H-pyrrole-1-carboxylate, 5 mL of 1,4-dioxane and 2.8 mL of 2.0 molar potassium carbonate aqueous solution. The flask was swept with nitrogen for five minutes and heated at 95° C. for 18 hours. The reaction and the reaction was allowed to cool and diluted with 15 mL water and 15 mL of ethyl acetate added over two minutes. No solids formed and organic phase was dried and concentrated under vacuum to yield 610 mg (quantitative yield).

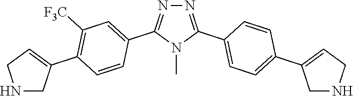 [SMILES: Cn1c(-c2ccc(C3=CCNC3)cc2)nnc1-c1ccc(C2=CCNC2)c(C(F)(F)F)c1]

Synthesis of 3-(4-(2,5-Dihydro-1H-pyrrol-3-yl)-3-(trifluoromethyl)phenyl)-5-(4-(2,5-dihydro-1H-pyrrol-3-yl)phenyl)-4-methyl-4H-1,2,4-triazole: A flask was charged with 610 mg (0.90 mmol) of tert-butyl 3-(4-(5-(4-(1-(tert-butoxycarbonyl)-2,5-dihydro-1H-pyrrol-3-yl)-3-(trifluoromethyl)phenyl)-4-methyl-4H-1,2,4-triazol-3-yl)phenyl)-2,5-dihydro-1H-pyrrole-1-carboxylate which was dissolved with 800 mg (7.41 mmol) of anisole and 8 mL dichloromethane. Then 12 mL (156 mmol) of trifluoroacetic acid added over five minutes with stirring. Three hours later, the reaction was concentrated under vacuum and triturated with two 30 mL portions of ether and ether decanted away to yield a solid in quantitative yield.

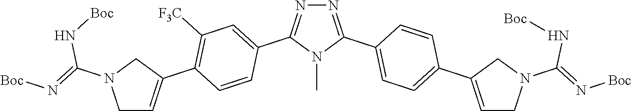 [SMILES: Cn1c(-c2ccc(C3=CCN(/C(=N/C(=O)OC(C)(C)C)NC(=O)OC(C)(C)C)C3)cc2)nnc1-c1ccc(C2=CCN(/C(=N/C(=O)OC(C)(C)C)NC(=O)OC(C)(C)C)C2)c(C(F)(F)F)c1]

tert-Butyl N-[(3-{4-[5-(4-{1-[(E)-{[(tert-butoxy)carbonyl]-amino}({[(tert-butoxy)carbonyl]imino})-methyl]-2,5-dihydro-1H-pyrrol-3-yl}-3-(trifluoromethyl)phenyl)-4-methyl-4H-1,2,4-triazol-3-yl]phenyl}-2,5-dihydro-1H-pyrrol-1-yl)({[(tert-butoxy)carbonyl] imino})methyl] carbamate: A flask was charged with 470 mg (0.70 mmol) of 3-(4-(2,5-dihydro-1H-pyrrol-3-yl)-3-(trifluoromethyl)phenyl)-5-(4-(2,5-dihydro-1H-pyrrol-3-yl)phenyl)-4-methyl-4H-1,2,4-triazole.bis trifluoroacetic acid salt and 7 mL N,N-dimethylformamide. Then 850 mg (8.40 mmol) of triethylamine added over two minutes. Then 950 mg (3.06 mmol) of tert-butyl (1H-pyrazol-1-yl)methanediylidenedicarbamate was added at once. After stirring for 18 hours the reaction was diluted with 30 mL ethyl acetate and then washed with three portions of 20 mL of water. Organic phase was dried with sodium sulfate and concentrated under vacuum. Yield was 700 mg of product in quantitative yield.

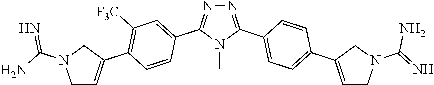 [SMILES: Cn1c(-c2ccc(C3=CCN(C(=N)N)C3)cc2)nnc1-c1ccc(C2=CCN(C(=N)N)C2)c(C(F)(F)F)c1]

### Example 27

3-(4-(5-(4-(1-Carbamimidoyl-2,5-dihydro-1H-pyrrol-3-yl)-3-(trifluoromethyl)phenyl)-4-methyl4H-1,2,4-triazol-3-yl)phenyl)-2,5-dihydro-1H-pyrrole-1-carboximidamide: A flask was charged with 510 mg (0.663 mmol) of tert-butyl N-[(3 {4-[5-(4-{1-[(E)-{[(tert-butoxy)carbonyl]amino}-({[(tert-butoxy)carbonyl]imino})methyl]-2,5-dihydro-1H-pyrrol-3-yl}-3-(trifluoromethyl)phenyl)-4-methyl-4H-1,2,4-triazol-3-yl]phenyl}-2,5-dihydro-1H-pyrrol-1-yl)({([(tert-butoxy)carbonyl]imino})methyl]-carbamate which was dissolved with a mixture of 500 mg (4.63 mmol) of anisole and 4 mL of dichloromethane. Then 6 mL (77.9 mmol) of trifluoroacetic acid added over two minutes with stirring and stirred for 18 hours. The LCMS indicted reaction complete and reaction concentrated under vacuum. Oil was triturated with two 30 mL portions of ether and ether decanted away to yield a solid. Solid was dissolved in N,N-dimethylformamide and was purified by prep HPLC. Yield was 83 mg (23%). ¹H NMR (300 MHz, DMSO-d₆) δ=8.23-8.07 (m, 2H), 7.92-7.72 (m, 2H), 7.66 (d, J=8.1 Hz, 2H), 7.40 (br d, J=5.7 Hz, 8H), 6.68 (s, 1H), 6.12 (s, 1H), 4.63 (s, 2H), 4.52 (br s, 2H), 4.40 (br s, 3H), 3.75 (s, 21≥), 3.67-3.55 (m, 2H). LC/MS method A: R_{f}=2.75 mins., (M+H)⁺==522,251, purity >95%.

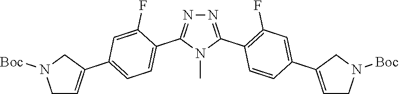 [SMILES: Cn1c(-c2ccc(C3=CCN(C(=O)OC(C)(C)C)C3)cc2F)nnc1-c1ccc(C2=CCN(C(=O)OC(C)(C)C)C2)cc1F]

Synthesis of tert-Butyl 3,3′-(4,4′-(4-methyl-4H-1,2,4-triazole-3,5-diyl)bis(3-fluoro-4,1-phenylene))bis(2,5-dihydro-1H-pyrrole-1-carboxylate): A flask was charged with 350 mg (0.812 mmol) of 3,5-Bis(4-bromo-2-fluorophenyl)-4-methyl-4H-1,2,4-triazole, 15 mg (0.065 mmol) of palladium acetate, 67 mg (0.162 mmol) of 2-dicyclohexylphosphino-2′,6′-dimethoxybiphenyl, 575 mg (2.04 mmol) of tert-butyl 3-(4,4,5,5-tetramethyl-1,3,2-dioxaborolan-2-yl)-2,5-dihydro-1H-pyrrole-1-carboxylate, 3.5 mL of 1,4-dioxane and 3 mL of 2.0 molar potassium carbonate aqueous solution. The flask was swept with nitrogen for five minutes and heated at 95° C. for 18 hours. The reaction was allowed to cool and diluted with 20 mL water and 15 mL of ethyl acetate added over two minutes. Limited solids formed and organic phase was dried and concentrated under vacuum and combined with solids to yield 550 mg (quantitative yield).

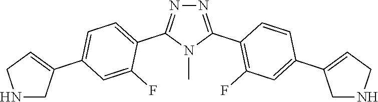 [SMILES: Cn1c(-c2ccc(C3=CCNC3)cc2F)nnc1-c1ccc(C2=CCNC2)cc1F]

Synthesis of 3,5-bis(4-(2,5-Dihydro-1H-pyrrol-3-yl)-2-fluorophenyl)-4-methyl-4H-1,2,4-triazole: A flask was charged with 550 mg (0.80 mmol) of tert-butyl 3,3′-(4,4′-(4-methyl-4H-1,2,4-triazole-3,5-diyl)bis(3-fluoro-4,1-phenylene))bis(2,5-dihydro-1H-pyrrole-1-carboxylate) which was dissolved with 500 mg (4.62 mmol) of anisole and 9 mL dichloromethane. Then 12 mL (156 mmol) of trifluoroacetic acid added over five minutes with stirring. Three hours later; the reaction was concentrated under vacuum and triturated with two 30 mL portions of ether and ether decanted away to yield a solid in quantitative yield.

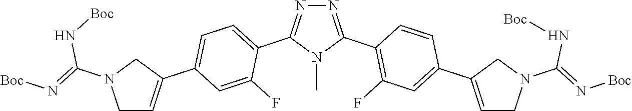 [SMILES: Cn1c(-c2ccc(C3=CCN(/C(=N/C(=O)OC(C)(C)C)NC(=O)OC(C)(C)C)C3)cc2F)nnc1-c1ccc(C2=CCN(/C(=N/C(=O)OC(C)(C)C)NC(=O)OC(C)(C)C)C2)cc1F]

Synthesis of tert-Butyl (3,3′-(4,4′-(4-methyl-4H-1,2,4-triazole-3,5-diyl)bis(3-fluoro-4,1-phenylene))bis(1H-pyrrole-3,1(2H,5H)-diyl))bis(methane-1-yl-1,1-diylidene) tetracarbamate: A flask was charged with 260 mg (0.40 mmol) of 3,5-bis(4-(2,5-dihydro-1H-pyrrol-3-yl)-2-fluorophenyl)-4-methyl-4H-1,2,4-triazole bis trifluoroacetic acid salt and 4 mL N,N-dimethylformamide. Then 404 mg (4.00 mmol) of triethylamine added over two minutes. Then 322 mg (0.1.04 mmol) of tert-butyl (0.1H-pyrazol-1-yl)methanediylidene dicarbamate was added at once. After stirring for 18 hours the reaction was diluted with 50 mL ethyl acetate and then 20 mL of water. Aqueous phase extracted with 30 mL dichloromethane. Combined organic phases dried and concentrated under vacuum. Yield was 400 mg of product in quantitative yield.

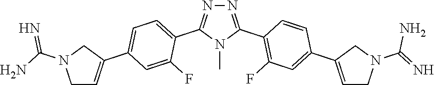 [SMILES: Cn1c(-c2ccc(C3=CCN(C(=N)N)C3)cc2F)nnc1-c1ccc(C2=CCN(C(=N)N)C2)cc1F]

### Example 28

Synthesis of 3,3′-(4,4′-(4-Methyl-4H-1,2,4-triazole-3,5-diyl)bis(3-fluoro-4,1-phenylene)) bis(2,5-dihydro-1H-pyrrole-1-carboximidamide): A flask was charged with 400 mg (0.40 mmol) of tert-butyl (3,3′-(4,4′-(4-methyl-4H-1,2,4-triazole-3,5-diyl)bis(3-fluoro-4,1-phenylene))bis(1H-pyrrole-3,1(2H,5H)-diyl))bis(methane-1-yl-1,1-diylidene)tetracarbamate which was dissolved with 6 mL of dichloromethane. Then 6 mL (77.9 mmol) of trifluoroacetic acid added over two minutes with stirring and stirred 18 hours. The LCMS indicted reaction complete and reaction concentrated under vacuum. Oil was triturated with two 30 mL portions of ether and ether decanted away to yield a solid. Solid was dissolved in N,N-dimethylformamide and was purified by prep HPLC. Yield was 230 mg (80%). ¹H NMR (300 MHz, DMSO-d₆) δ ppm 7.75-7.85 (m, 2H) 7.53-7.65 (m, 2H) 7.40-7.50 (m, 6H) 6.74-6.86 (m, 2H) 4.56-4.7.1 (m, 4H) 4.36-4.47 (m, 4H) 3.48 (m, 4H); LC/MS method A: R_{f}=3.03 mins., (M+H)⁺490, purity >95%.

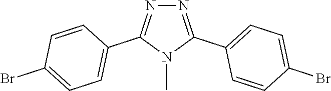 [SMILES: Cn1c(-c2ccc(Br)cc2)nnc1-c1ccc(Br)cc1]

Synthesis of 3,5-bis(4-Bromophenyl)-4-methyl-4H-1,2,4-triazole: A flask was charged with (Z)-4-bromo-N—((Z)-(4-bromophenyl)chloromethylene)benzohydrazonoyl chloride (4.0 grams, 9.2 mmol) was slurried with 5 mL of methanol and the methylamine (0.94 g, 30.4 mmol) was added over five minutes followed by 0.82 grams (13.8 mmol) of glacial acetic acid was added over five minutes. The reaction was heated at 70° C. for 18 hours. The reaction was allowed to cool and most of methanol removed under vacuum. Then 40 mL of water added over several minutes. One hour later the solid was collected, air dried for 15 minutes and rinsed with ten mL of water, ten mL of ether and air dried in the hood for several hours. Yield was 3.4 grams (68%).

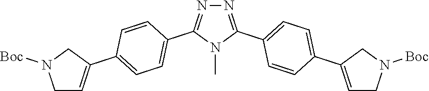 [SMILES: Cn1c(-c2ccc(C3=CCN(C(=O)OC(C)(C)C)C3)cc2)nnc1-c1ccc(C2=CCN(C(=O)OC(C)(C)C)C2)cc1]

Synthesis of tert-Butyl 3,3′-(4,4′-(4-methyl-4H-1,2,4-triazole-3,5-diyl)bis(4,1-phenylene))bis(2,5-dihydro-1H-pyrrole-1-carboxylate): A flask was charged with 250 mg (0.63 mmol) of 3,5-bis(4-bromophenyl)-4-methyl-4H-1,2,4-triazole, 11.4 mg (0.05 mmol) of palladium acetate, 52 mg (0.12 mmol) of 2-dicyclohexylphosphino-2′,6′-dimethoxybiphenyl, 450 mg (1.52 mmol) of tert-butyl 4-(4,4,5,5-tetramethyl-1,3,2-dioxaborolan-2-yl)-5,6-dihydropyridine-1(2H)-carboxylate, 2.0 mL of 1,4-dioxane and 1.0 mL of 2.0 molar potassium carbonate aqueous solution. The flask was swept with nitrogen for five minutes and heated at 95° C. for 18 hours. Then the reaction was complete by LCMS and the reaction was allowed to cool and diluted with 4 mL water and 10 mL of ethyl acetate added over ten minutes. Solids formed quickly and reaction stirred for two hours. Solid collected on a filter and rinsed with 30 mL ethyl acetate and air-dried for 18 hours in the hood to yield 281 mg (66%).

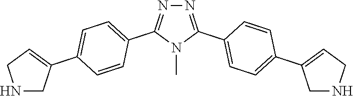 [SMILES: Cn1c(-c2ccc(C3=CCNC3)cc2)nnc1-c1ccc(C2=CCNC2)cc1]

Synthesis of 3,5-bis(4-(2,5-Dihydro-1H-pyrrol-3-yl)phenyl)-4-methyl-4H-1,2,4-triazole: di-tert-butyl 3,3′-((4-methyl-4H-1,2,4-triazole-3,5-diyl)bis(4,1-phenylene))bis(2,5-dihydro-1H-pyrrole-1-carboxylate) 281 mg (0.29 mmol) was slurried with 0.5 mL of anisole and 1.5 mL dichloromethane. Then 3.0 mL (40 mmol) of trifluoroacetic acid added over five minutes with stirring. The reaction was concentrated under vacuum and triturated with two 5 mL portions of ether and ether decanted away to yield a solid in quantitative yield.

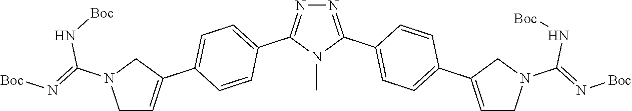 [SMILES: Cn1c(-c2ccc(C3=CCN(/C(=N/C(=O)OC(C)(C)C)NC(=O)OC(C)(C)C)C3)cc2)nnc1-c1ccc(C2=CCN(/C(=N/C(=O)OC(C)(C)C)NC(=O)OC(C)(C)C)C2)cc1]

Synthesis of tert-Butyl (3,3′-(4,4′-(4-methyl-4H-1,2,4-triazole-3,5-diyl)bis(4,1-phenylene)) bis(1H-pyrrole-3,1(2H,5H)-diyl))bis(methane-1-yl-1,1-diylidene)tetracarbamate: A flask was charged with 100 mg (0.27 mmol) of 3,5-bis(4-(2,5-dihydro-1H-pyrrol-3-yl)phenyl)-4-methyl-4H-1,2,4-triazole bis trifluoroacetic acid salt and 1.0 mL N,N-dimethylformamide. Then 164 mg (1.62 mmol) of triethylamine added.

Then 252 mg (0.81 mmol) of tert-butyl (1H-pyrazol-1-yl)methanediylidenedicarbamate was added. The reaction slurry was warmed at 40° C. for 18 hours. The reaction was diluted with 20 mL ethyl acetate and then washed with four portions of 5 mL of water. Organic phase was dried with sodium sulfate and concentrated under vacuum. The procut was purified by normal phase chromatography, loaded as dichloromethane solution, eluted with (30% acetonitrile/dichloromethane)/dichloromethane with step gradient from 0 to 100% to elute impurities. Then the expected product eluted off with (20% MeOH/dichloromethane)/dichloromethane step gradient from 0 to 100%. Yield was 79 mg of product (34%).

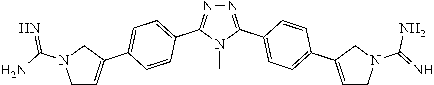 [SMILES: Cn1c(-c2ccc(C3=CCN(C(=N)N)C3)cc2)nnc1-c1ccc(C2=CCN(C(=N)N)C2)cc1]

### Example 29

Synthesis of 3,3′-(4,4′-(4-Methyl-4H-1,2,4-triazole-3,5-diyl)bis(4,1-phenylene))bis(2,5-dihydro-1H-pyrrole-1-carboximidamide: A solution of tert-butyl ((Z)-(3-(4-(5-(4-(1-((E)-N,N′-bis(tert-butoxycarbonyl)carbamimidoyl)-2,5-dihydro-1H-pyrrol-3-yl)phenyl)-4-methyl-4H-1,2,4-triazol-3-yl)phenyl)-2,5-dihydro-1H-pyrrol-1-yl)((tert-butoxycarbonyl)amino)methylene) carbamate (79.4 mg, 0.09 mmol) was dissolved with a mixture of 0.5 mL of anisole and 4 mL of dichloromethane. Then 4 mL (52 mmol) of trifluoroacetic acid added over two minutes with stirring and stirred for 18 hours. The next day LCMS indicted reaction complete and reaction concentrated under vacuum. Oil was triturated with two 10 mL portions of ether and ether decanted away to yield a solid product. Yield was 40 mg (95%). ¹H NMR (300 MHz, DMSO-d₆) δ=8.07-7.82 (m, 4H), 7.77-7.54 (m, 4H), 7.48-7.29 (m, 8H), 6.67 (s, 2H), 4.63 (br d, J=4.5 Hz, 2H), 3.93 (br d, J=5.4 Hz, 2H), 3.87-3.75 (m, 2H), 3.72-3.25 (m, 2H). LC/MS method A: R_{f}=3.23 mins., (M+H)⁺=455, purity >95%.

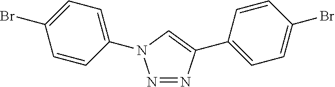 [SMILES: Brc1ccc(-c2cn(-c3ccc(Br)cc3)nn2)cc1]

Synthesis of 1,4-Bis(4-bromophenyl)-1H-1,2,3-triazole A flask was charged with 252 mg (1.00 mol) of ((4-bromophenyl)ethynyl)trimethylsilane, 16 mg (0.10 mmol) of copper 11 sulfate, 40 mg (0.20 mmol) of sodium ascorbate and 221 mg (3.8 mmol) of potassium fluoride. Then 5 mL of water, 5 mL of methanol and 5 mL tetrahydrofuran added. Then 2 mL of 0.5 M solution of 1-azido-4-bromo-benzene in tetrahydrofuran was added and reaction stirred for four hours at 23° C. and heated at 35° C. for 18 hours. The reaction was cooled and solids collected and rinsed with 2 mL of methanol. After dry in for several hours yield was 302 m 80%

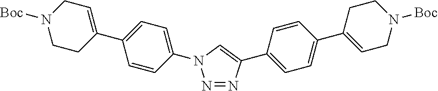 [SMILES: CC(C)(C)OC(=O)N1CC=C(c2ccc(-c3cn(-c4ccc(C5=CCN(C(=O)OC(C)(C)C)CC5)cc4)nn3)cc2)CC1]

Synthesis of tert-Butyl 4-{4-[1-(4{-1-[(tert-butoxy)carbonyl]-1,2,3,6-tetrahydropyridin-4-yl}phenyl)-1H-1,2,3-triazol-4-yl]phenyl}-1,2,3,6-tetrahydropyridine-1-carboxylate: A flask was charged with 360 mg (0.955 mmol) of 1,4-bis(4bromophenyl)-1H-1,2,3-triazole, 11 mg (0.048 mmol) of palladium acetate, 53 mg (0.132 mmol) of 2-dicyclohexylphosphino-2′,6′-dimethoxybiphenyl, 628 mg (2.05 mmol) of tert-butyl 4-(4,4,5,5-tetramethyl-1,3,2-dioxaborolan-2-yl)-5,6-dihydropyridine-1(2H)-carboxylate, 8 mL of 1,4-dioxane and 3.5 mL of 2.0 molar potassium carbonate aqueous solution. The flask was swept with nitrogen for seven minutes and heated at 95° C. for 18 hours. The reaction was complete by LCMS and the reaction was allowed to cool and diluted with 10 mL water and 20 mL of ethyl acetate added over two minutes. Solids formed quickly and reaction stirred for one hours. Solid triturated with a mixture of 1,4-dioxane 6 mL and methanol 3 mL and then 20 mL of 20% methanol/dioxane. Triterations combined, filtered and concentrated under vacuum to yield 361 mg (65%) of product.

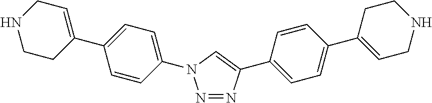 [SMILES: C1=C(c2ccc(-c3cn(-c4ccc(C5=CCNCC5)cc4)nn3)cc2)CCNC1]

Synthesis of 4(4-{1-[4-(1,2,3,6-Tetrahydropyridin-4-yl)phenyl]-1H-1,2,3-triazol-4-yl}phenyl)1,2,3,6-tetrahydropyridine: A flask was charged with 361 mg (0.619 mmol) of the tert-butyl 4-{4-[1-(4-{1-[(tert-butoxy)carbonyl]-1,2,3,6-tetrahydropyridin-4-yl}phenyl)-1H-1,2,3-triazol-4-yl]phenyl}-1,2,3,6-tetrahydropyridine-1-carboxylate was slurried with 3 mL dichloromethane. Then 7 mL (91 mmol) of trifluoroacetic acid added over three minutes with stirring. Three hours later the reaction was concentrated under vacuum and triturated with 20 mL of ethyl acetate which was decanted away to yield a solid in quantitative yield.

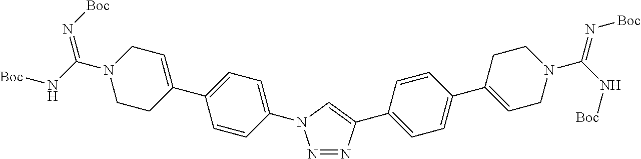 [SMILES: CC(C)(C)OC(=O)/N=C(/NC(=O)OC(C)(C)C)N1CC=C(c2ccc(-n3cc(-c4ccc(C5=CCN(/C(=N/C(=O)OC(C)(C)C)NC(=O)OC(C)(C)C)CC5)cc4)nn3)cc2)CC1]

Synthesis of tert-Butyl N-[(E)-(4-{4-[1-(4-{1-[(Z)-{[(tert-butoxy)carbonyl]amino}-({[(tert-butoxy)carbonyl]imino})methyl]-1,2,3,6-tetrahydropyridin-4-yl}phenyl)-1H-1,2,3-triazol-4-yl]phenyl}-1,2,3,6-tetrahydropyridin-1-yl)({[(tert-butoxy)carbonyl]imino})methyl]carbamate: A flask was charged with 306 mg (0.50 mmol) of 4-(4-{1-[4-(1,2,3,6-tetrahydropyridin-4-yl)phenyl]-1H-1,2,3-triazol-4-yl}phenyl)-1,2,3,6-tetrahydropyridine bis trifluoroacetic acid salt, 3 mL N,N-dimethylformamide and 3 mL dichloromethane. Then 560 mg (5.55 mmol) of triethylamine added over five minutes. Then 341 mg (1.11 mmol) of tert-butyl (1H-pyrazol-1-yl)methanediylidenedicarbamate was added. After stirring for 18 hours at room temperature, the reaction was concentrated under vacuum and diluted with 25 mL ethyl acetate and then washed with 10 mL of water. Organic phase was dried with sodium sulfate and concentrated under vacuum. Purified by normal phase chromatography, loaded as dichloromethane solution, eluted with (30% acetonitrile/dichloromethane)/dichloromethane with step gradient from 0 to 100% to elute product. Yield was 144 mg of product (33%)

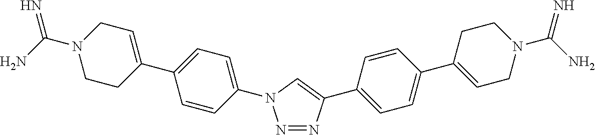 [SMILES: N=C(N)N1CC=C(c2ccc(-c3cn(-c4ccc(C5=CCN(C(=N)N)CC5)cc4)nn3)cc2)CC1]

### Example 30

Synthesis of 4(4-{1-[4-(1-Carbamimidoyl-1,2,3,6-tetrahydropyridin-4-yl) phenyl]-1H-1,2,3-triazol-4-yl}phenyl)-1,2,3,6-tetrahydropyridine-1-carboximidamide: A flask was charged with 144 mg (0.166 mmol) of tert-butyl N-[(E)-(4-{4-[1-(4-{1-[(Z)-{[(tert-butoxy)carbonyl]amino}({[(tert-butoxy)carbonyl]imino})methyl]-1,2,3,6-tetrahydropyridin-4-yl}phenyl)-1H-1,2,3-triazol-4-yl]phenyl}-1,2,3,6-tetrahydropyridin-1-yl)({[(tert-butoxy)carbonyl]imino}) methyl]carbamate which was dissolved with 4 mL of dichloromethane. Then 4 mL (52 mmol) of trifluoroacetic acid added over two minutes with stirring and stirred for 18 hours. The next day LCMS indicted reaction complete and reaction concentrated under vacuum. Oil was triturated with two 20 mL portions of ethyl acetate which was decanted away to yield a solid. Solid was dissolved dried under vacuum at 45° C. for 18 hours. Yield was 81 mg (70%). ¹H NMR (300 MHz, DMSO-d₆) δ=9.33 (s, 1H), 7.98-7.89 (m, 4H), 7.73 (d, J=8.8 Hz, 1H), 7.61 (d, J=8.5 Hz, 1H), 7.52-7.34 (m, 2H), 6.38-6.27 (m, 2H), 4.16-3.97 (m, 4H), 3.72-3.56 (m, 4H), 3.45-3.36 (m, 1H), 3.27-3.21 (m, 1H), 1H), 2.72-2.60 (m, 1H), 2.59-2.51 (m, 1H), 2.44-2.34 (m, 1H). LC/MS method A: R_{f}=3.29 mins., (M+H)⁺=469, purity >95%.

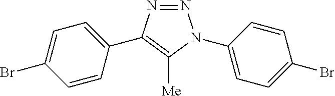 [SMILES: Cc1c(-c2ccc(Br)cc2)nnn1-c1ccc(Br)cc1]

1,4-Bis(4-bromophenyl)-5-methyl-1H-1,2,3-triazole was made from (Z)—N′-(1-(4-bromophenyl)propylidene)benzenesulfonohydrazide and 4-bromoaniline. (Z)—N′-(1-(4-Bromophenyl)propylidene)benzenesulfonohydrazide was prepared from 1-(4-bromophenyl)propan-1-one and benzenesulfonohydrazide.

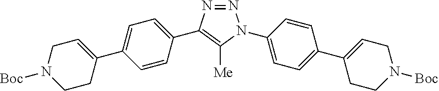 [SMILES: Cc1c(-c2ccc(C3=CCN(C(=O)OC(C)(C)C)CC3)cc2)nnn1-c1ccc(C2=CCN(C(=O)OC(C)(C)C)CC2)cc1]

tert-Butyl 4-{4-[1-(4-{1-[(tert-butoxy)carbonyl]-1,2,3,6-tetrahydro-pyridin-4-yl}phenyl)-5-methyl-1H-1,2,3-triazol-4-yl]phenyl}-1,2,3,6-tetrahydropyridine-1-carboxylate was prepared from 1,4-bis(4-bromophenyl)-5-methyl-1H-1,2,3-triazole and tert-butyl 4-(4,4,5,5-tetramethyl-1,3,2-dioxaborolan-2-yl)-5,6-dihydropyridine-1(2H)-carboxylate.

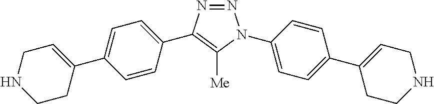 [SMILES: Cc1c(-c2ccc(C3=CCNCC3)cc2)nnn1-c1ccc(C2=CCNCC2)cc1]

4-(4-{5-Methyl-1-[4-(1,2,3,6-tetrahydropyridin-4-yl)phenyl]-1H-1,2,3-triazol-4-yl}phenyl)-1,2,3,6-tetrahydropyridine was prepared from tert-butyl 4-{4-[1-(4 {1-[(tert-butoxy)carbonyl]-1,2,3,6-tetrahydropyridin-4-yl}phenyl)-5-methyl-1H-1,2,3-triazol-4-yl]phenyl}-1,2,3,6-tetrahydropyridine-1-carboxylate.

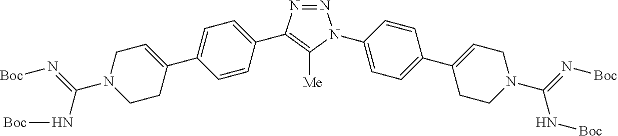 [SMILES: Cc1c(-c2ccc(C3=CCN(/C(=N/C(=O)OC(C)(C)C)NC(=O)OC(C)(C)C)CC3)cc2)nnn1-c1ccc(C2=CCN(/C(=N/C(=O)OC(C)(C)C)NC(=O)OC(C)(C)C)CC2)cc1]

tert-Butyl N-[(E)-(4-{4-[1-(4-{1-[(E)-{[(tert-butoxy)-carbonyl]amino}({[(tert-butoxy)carbonyl]imino})methyl]-1,2,3,6-tetrahydropyridin-4-yl}phenyl)-5-methyl-1H-1,2,3-triazol-4-yl]phenyl}-1,2,3,6-tetrahydropyridin-1-yl)({[(2-hydroxypropan-2-yl)oxy]carbonyl}imino)methyl]carbamate was prepared from 4-(4-{5-methyl-1-[4-(1,2,3,6-tetrahydro-pyridin-4-yl)phenyl]-1H-1,2,3-triazol-4-yl}phenyl)-1,2,3,6-tetrahydropyridine.

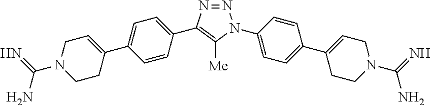 [SMILES: Cc1c(-c2ccc(C3=CCN(C(=N)N)CC3)cc2)nnn1-c1ccc(C2=CCN(C(=N)N)CC2)cc1]

### Example 31

4-(4-{1-[4-(1-Carbamimidoyl-1,2,3,6-tetrahydropyridin-4-yl)phenyl]-5-methyl-1H-1,2,3-triazol-4-yl}phenyl)-1,2,3,6-tetrahydropyridine-1-carboximidamide was prepared from tert-butyl N-[(E)-(4-{4-[1-(4-{1-[(E)-{[(tert-butoxy)carbonyl]amino}({[(tert-butoxy)-carbonyl] imino})methyl]-1,2,3,6-tetrahydropyridin-4-yl}phenyl)-5-methyl-1H-1,2,3-triazol-4-yl]phenyl}-1,2,3,6-tetrahydropyridin-1-yl)({[(2-hydroxypropan-2-yl)oxy]carbonyl}imino)methyl]carbamate. ¹H NMR. (300 MHz, METHANOL-d₄) δ=7.92-7.66 (m, 8H), 6.44-6.21 (m, 2H), 3.96-3.86 (m, 4H), 3.8.1-3.65 (m, 4H), 3.58-3.32 (m, 4H), 2.77 (s, 3H). LC/MS method A: R_{f}:=2.64 mins., (M+H)⁺=−482, purity >95%.

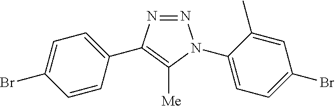 [SMILES: Cc1cc(Br)ccc1-n1nnc(-c2ccc(Br)cc2)c1C]

1-(4-Bromo-2-methylphenyl)-4-(4-bromophenyl)-5-methyl-1H-1,2,3-triazole was prepared from (Z)—N′-(1-(4-bromophenyl)propylidene)-benzenesulfonohydrazide and 4-bromo-2-methylaniline. (Z)—N-(1-(4-Boronophenyl)propylidene)benzenesulfonohydrazide was prepared from 1-(4-bromophenyl)propan-1-one and benzenesulfonohydrazide.

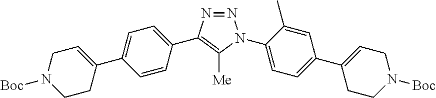 [SMILES: Cc1cc(C2=CCN(C(=O)OC(C)(C)C)CC2)ccc1-n1nnc(-c2ccc(C3=CCN(C(=O)OC(C)(C)C)CC3)cc2)c1C]

tert-Butyl 4-(4-(1-(4-(1-(tert-butoxycarbonyl)-1,2,3,6-tetrahydropyridin-4-yl)-2-methylphenyl)-5-methyl-1H-1,2,3-triazol-4-yl)phenyl)-5,6-dihydropyridine-1(2H)-carboxylate was prepared from 1-(4-bromo-2-methylphenyl)-4-(4-bromophenyl)-5-methyl-1H-1,2,3-triazole and tert-butyl 4-(4,4,5,5-tetramethyl-1,3,2-dioxaborolan-2-yl)-5,6-dihydropyridine-1(2H)-carboxylate.

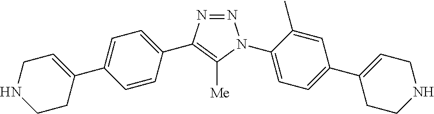 [SMILES: Cc1cc(C2=CCNCC2)ccc1-n1nnc(-c2ccc(C3=CCNCC3)cc2)c1C]

4-(4-(5-Methyl-1-(2-methyl-4-(1,2,3,6-tetrahydropyridin-4-yl)phenyl)-1H-1,2,3-triazol-4-yl)phenyl)-1,2,3,6-tetrahydropyridine was prepared from tert-butyl 4-(4-(1-(4-(1-(tert-butoxycarbonyl)-1,2,3,6-tetrahydropyridin-4-yl)-2-methylphenyl)-5-methyl-1H-1,2,3-triazol-4-yl)phenyl)-5,6-dihydropyridine-1. (2H)-carboxylate.

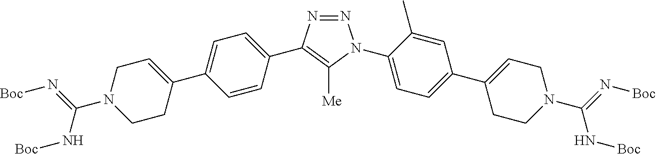 [SMILES: Cc1cc(C2=CCN(/C(=N/C(=O)OC(C)(C)C)NC(=O)OC(C)(C)C)CC2)ccc1-n1nnc(-c2ccc(C3=CCN(/C(=N/C(=O)OC(C)(C)C)NC(=O)OC(C)(C)C)CC3)cc2)c1C]

tert-Butyl N—[(Z)-(4-{4-[4-(4-{1-[(E)-{[(tert-butoxy)carbonyl]-amino}({[(tert-butoxy)carbonyl]imino})methyl]-1,2,3,6-tetrahydropyridin-4-yl}phenyl)-5-methyl-1H-1,2,3-triaz-ol-1-yl]-3-methylphenyl}-1,2,3,6-tetrahydropyridin-1-yl)({[(tert-butoxy)carbonyl]imino})methyl] carbamate was prepared from 4-(4-(5-methyl-1-(2-methyl-4-(1,2,3,6-tetrahydropyridin-4-yl)phenyl)-1H-1,2,3-triazol-4-yl)phenyl)-1,2,3,6-tetrahydropyridine.

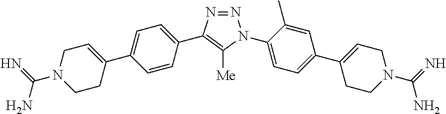 [SMILES: Cc1cc(C2=CCN(C(=N)N)CC2)ccc1-n1nnc(-c2ccc(C3=CCN(C(=N)N)CC3)cc2)c1C]

### Example 32

4-(4-(1-(4-(1-Carbamimidoyl-1,2,3,6-tetrahydropyridin-4-yl)-2-methyl phenyl)-5-methyl-1H-1,2,3-triazol-4-yl)phenyl)-5,6-dihydropyridine-1(2H)-carboximidamide was prepared from tert-butyl N—[(Z)-(4-{4-[4-(4-{1-[(E)-{[(tert-butoxy)carbonyl]amino}-({[(tert-butoxy) carbonyl]imino})methyl]-1,2,3,6-tetrahydropyridin-4-yl}phenyl)-5-methyl-1H-1,2,3-triazol-1-yl]-3-methylphenyl}-1,2,3,6-tetrahydropyridin-1-yl)({[(tert-butoxy)carbonyl]imino})methyl.]carbamate. ¹H NMR (300 MHz, DMSO-d₆) δ=7.83-7.78 (m, 1H), 7.65-7.51. (m, 3H), 7.48-7.36 (m, 5H), 6.37 (br t, J=3.4 Hz, 1H), 6.30 (br t, J=3.1 Hz, 1H), 4.13-4.08 (m, 4H), 3.67-3.61 (m, 4H), 2.68-2.61 (m, 4H), 2.30 (s, 3H), 2.03 (s, 3H). LC/MS method A: R_{f}=3.13 mins., (M+H)⁺=497, purity >95%.

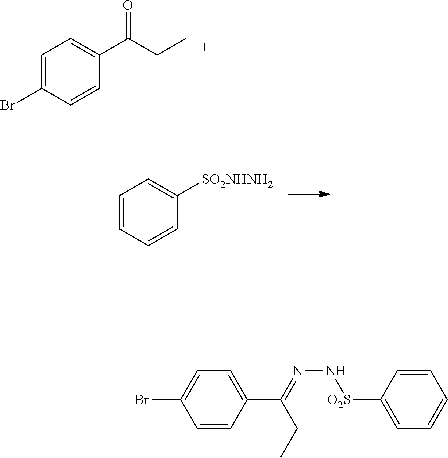

Synthesis of N′-[(1E)-1-(4-Bromophenyl)ethylidene]benzenesulfonohydrazide: A solution of phenylsulfonylhydrazide (2.1 g, 12 mmol) was dissolved in methanol (10 mL) and the solution was treated with 4-bromopropioophenone (2.4 g, 12 mmol). After stirring for 30 minutes a thick precipitate formed. Additional methanol (10 mL) was added and the mixture was filtered, washed with methanol, and dried under vacuum to leave 3.6 g (85%) of white solid which was a mixture of syn and anti isomers. ¹H-NMR (CDCl₃) δ8.03 (d, 1H, J=8.5 Hz), 7.92 (d, 0.5H; J=8.5 Hz), 7.82 (d, 0.5H, J=8.5 Hz), 7.45-7.65 (m, 7H), 2.60 and 2.15 (s, 3H). LCMS method A R_{f}=5.30 mins, purity >95%, (M+H)⁺=354.

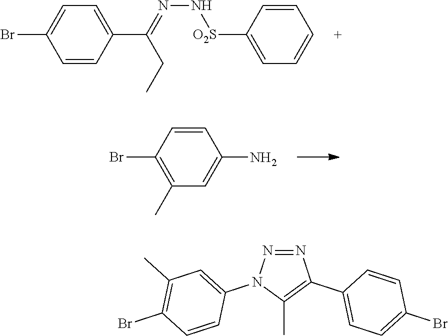

Synthesis of 1-(4-Bromo-3-methylphenyl)-4-(4-bromophenyl)-5-methyl-1H-1,2,3-triazole: A mixture of N′-[(1E)-1-(4-bromophenyl)ethylidene]benzenesulfonohydrazide (530 mg, 1.5 mmol), 4-bromo-3-methylaniline (561 mg, 3.0 mmol), copper(II) acetate (273 mg, 1.5 mmol) and pivalic acid (3.5 mg, 3.0 mmol) in toluene (15 mL) was stirred and heated to 100° C. for 18 hours open to the atmosphere. The mixture was cooled to 20° C., diluted with dichloromethane (15 mL) and evaporated onto silica gel. The crude product was purified by silica gel chromatography eluted with a gradient of 50% hexanes in dichloromethane to 100% dichloromethane to leave the product as a tan solid (78 mg, 12%). ¹H-NMR (CDCl₃) □ 7.75-7.50 (m, 6H), 7.16 (d, 1H, J=8.2 Hz), 2.34 (s, 3H), 2.08 (s, 3H). LCMS method A R_{f}=6.19 mins, purity >95%, (M+H)⁺=408.

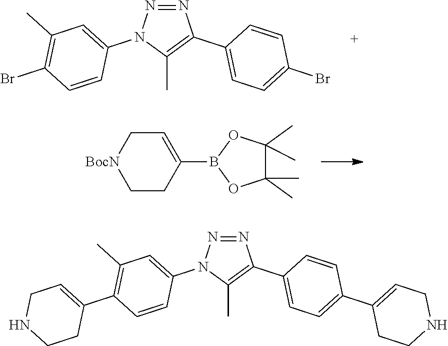

Synthesis of 4-(2-Methyl-4-(5-methyl-4-[4-(1,2,3,6-tetrahydropyridin-4-yl)phenyl]-1H-1,2,3-triazol-1-yl)phenyl)-1,2,3,6-tetrahydropyridine: A mixture of 1-(4-bromo-3-methylphenyl)-4-(4-bromophenyl)-5-methyl-1H-1,2,3-triazole (50 mg, 0.12 mmol), tert-butyl 4-(4,4,5,5-tetramethyl-1,3,2-dioxaborolan-2-yl)-1,2,3,6-tetrahydropyridine-1-carboxylate (93 mg, 0.30 mmol), dicyclohexyl(2′,6′-dimethoxybiphenyl-2-yl) phosphine (8 mg, 20 (mol), palladium acetate (2.0 mg, 7 □mol), potassium carbonate (110 mg, 0.80 mmol), water (0.4 mL) and 1,4-dioxane (1 mL) was purged with nitrogen, stirred and heated to 90° C. for 20 hours. The mixture was diluted with dichloromethane (20 mL) and water (20 mL) and the aqueous layer was extracted with dichloromethane (20 mL). The combined dichloromethane layers were dried (Na₂SO₄) and evaporated. The crude product mixture was purified by silica gel chromatography eluted with a gradient of 15% ethyl acetate in hexanes to 50% ethyl acetate in hexanes to leave the product as a gum (21 mg, 29%). This was dissolved in trifluoroacetic acid (1 mL) and dichloromethane (1 mL) and stirred for 2 hours. The mixture was evaporated and lyophilized to leave the product as a white solid (bis trifluoroacetic acid salt, 18 mg, 60%). ¹H-NMR (CD₃OD) δ 7.78 (d, 2H, J=8.2 Hz), 7.63 (d, 2H, J=8.2 Hz), 7.49 (s, 1H), 7.44 (d, 1H, J=8.5 Hz), 7.42 (d, 1H, J=8.5 Hz), 6.25 (m, 1H), 5.77 (m, 1H), 3.89 (m, 4H), 3.52 (m, 4H), 2.85 (m, 2H), 2.65 (m, 2H), 2.48 (s, 3H), 2.42 (s, 3H). LCMS method A R_{f}=2.67 mins, purity >95%, (M+H)⁺=412.

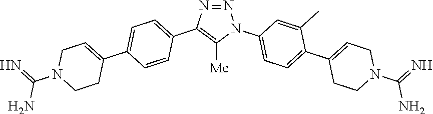 [SMILES: Cc1cc(-n2nnc(-c3ccc(C4=CCN(C(=N)N)CC4)cc3)c2C)ccc1C1=CCN(C(=N)N)CC1]

### Example 33

Synthesis of 4-(4-{4-[4-(1-Carbamimidoyl-1,2,3,6-tetrahydropyridin-4-yl)phenyl]-5-methyl-1H-1,2,3-triazol-1-yl}-2-methylphenyl)-1,2,3,6-tetrahydropyridne-1-carboximidamide: A solution of 4-(2-methyl-4-{5-methyl-4-[4-(1,2,3,6-tetrahydropyridin-4-yl) phenyl]-1H-1,2,3-triazol-1-yl}phenyl)-1,2,3,6-tetrahydropyridine (11 mg, 16 μmol) and N,N-diisopropyl ethylamine (20 mg, 0.16 mmol) in methanol (1 mL) was treated with N—((N′,N″-bis-tert-butyloxycarbonyl)amidino)pyrrazole (15 mg, 48 μmol) and stirred for 18 hours. N,N-dimethylformamide (1 mL) was added for solubilization and the mixture was purified by reversed phase HPLC. The product fractions were combined, treated with saturated sodium bicarbonate solution and extracted with dichloromethane (2×25 mL). The combined organic layers were dried (Na₂SO₄) and evaporated to a glassy solid which was dissolved in trifluoroacetic acid (1 mL) and dichloromethane (1 mL) and stirred for 2 hours. The solvents were evaporated and the product was lyophilized to a white solid (4.4 mg, 38%). ¹H-NMR (CD₃OD) δ 7.77 (d, 2H, J=8.3 Hz), 7.63 (d, 2H, J=8.3 Hz), 7.47 (s, 1H), 7.45 (d, 1H, J=8.5 Hz), 7.42 (d, 1H, J=8.5 Hz), 6.27 (m, 1H), 5.80 (m, 1H), 3.76 (m, 4H), 3.48 (m, 4H), 2.85 (m, 2H), 2.66 (m, 2H), 2.50 (s, 3H), 2.44 (s, 3H). LCMS method A R_{f}=2.52 mins, purity >95%, (M+H)⁺=496.

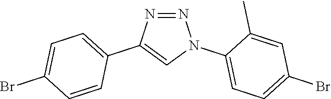 [SMILES: Cc1cc(Br)ccc1-n1cc(-c2ccc(Br)cc2)nn1]

1-(4-Bromo-2-methylphenyl)-4-(4-boronophenyl)-1H-1,2,3-triazole was from (Z)—N′-(1-(4-bromophenyl)ethylidene)-benzenesulfonoyl hydrazide and 4-bromo-2-methylaniline. (Z)—N′-(1-(4-bromophenyl)ethyl idene)-benzene sulfonohydrazide was prepared from 1-(4-bromophenyl)ethanone and benzenesulfonoyl hydrazide.

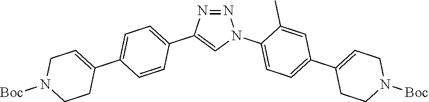 [SMILES: Cc1cc(C2=CCN(C(=O)OC(C)(C)C)CC2)ccc1-n1cc(-c2ccc(C3=CCN(C(=O)OC(C)(C)C)CC3)cc2)nn1]

tert-Butyl 4-(4-(1-(4-(1-(tert-butoxycarbonyl)-1,2,3,6-tetrahydropyridin-4-yl)-2-methylphenyl)-1H-1,2,3-triazol-4-yl)phenyl)-5,6-dihydropyridine-1 (2H)-carboxylate was prepared from 1-(4-bromo-2-methylphenyl)-4-(4-bromophenyl)-1H-1,2,3-triazole and tert-butyl 4-(4,4,5,5-tetramethyl-1,3,2-dioxaborolan-2-yl)-5,6-dihydropyridine-1(2H)-carboxylate.

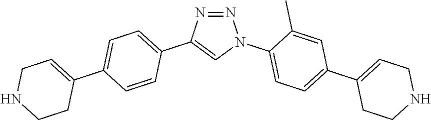 [SMILES: Cc1cc(C2=CCNCC2)ccc1-n1cc(-c2ccc(C3=CCNCC3)cc2)nn1]

4-(4-(1-(2-Methyl-4-(1,2,3,6-tetrahydropyridin-4-yl)phenyl)-1H-1,2,3-triazol-4-yl)phenyl)-1,2,3,6-tetrahydropyridine was prepared from tert-butyl 4-(4-(1-(4-(1-(tert-butoxycarbonyl)-1,2,3,6-tetrahydropyridin-4-yl)-2-methylphenyl)-1H-1,2,3-triazol-4-yl)phenyl)-5,6-dihydropyridine-1(2H)-carboxylate.

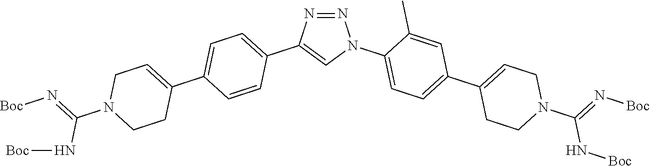 [SMILES: Cc1cc(C2=CCN(/C(=N/C(=O)OC(C)(C)C)NC(=O)OC(C)(C)C)CC2)ccc1-n1cc(-c2ccc(C3=CCN(/C(=N/C(=O)OC(C)(C)C)NC(=O)OC(C)(C)C)CC3)cc2)nn1]

tert-Butyl N—[(Z)-(4-{4-[4-(4-{1-[(E)-{[(tert-butoxy)carbonyl]amino}({[(tert-butoxy)carbonyl]imino})methyl]-1,2,3,6-tetrahydropyridin-4-yl}phenyl)-1H-1,2,3-triazol-1-yl]-3-methylphenyl}-1,2,3,6-tetrahydropyridin-1-yl)({[(tert-butoxy)carbonyl]imino})methyl] carbamate was prepared from 4-(4-(1-(2-methyl-4-(1,2,3,6-tetrahydropyridin-4-yl)phenyl)-1H-1,2,3-triazol-4-yl)phenyl)-1,2,3,6-tetrahydropyridine.

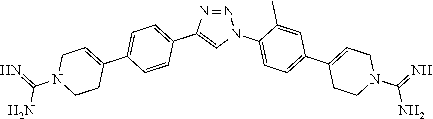 [SMILES: Cc1cc(C2=CCN(C(=N)N)CC2)ccc1-n1cc(-c2ccc(C3=CCN(C(=N)N)CC3)cc2)nn1]

### Example 34

4-(4-(1-(4-(1-Carbamimidoyl-1,2,3,6-tetrahydropyridin-4-yl)-2-methylphenyl)-1H-1,2,3-triazol-4-yl)phenyl)-5,6-dihydropyridine-1(2-H)-carboximidamide was prepared from tert-butyl N—[(Z)-(4-{4-[4-(4-{1-[(E)-{[(tert-butoxy)carbonyl]amino}({[(tert-butoxy) carbonyl]imino})methyl]-1,2,3,6-tetrahydropyridin-4-yl}phenyl)-11H-1,2,3-triazol-1-yl]-3-methyl phenyl}-1,2,3,6-tetrahydropyridin-1-yl)({[(tert-butoxy)carbonyl]imino})methyl]carbamate. ¹H NMR (300 MHz, CD₃OD) δ=8.61 (s, 1H), 8.16-7.79 (m, 2H), 7.71-7.33 (m, 5H), 6.44-6.13 (m, 2H), 4.34-4.01 (m, 4H), 3.90-3.62 (m, 4H), 2.88-2.76 (m, 4H), 2.29 (s, 3H). LC/MS method A: R_{f}=3.05 mins., (M+H)⁺=482, purity >95%.

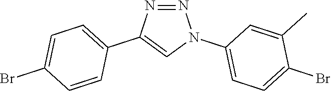 [SMILES: Cc1cc(-n2cc(-c3ccc(Br)cc3)nn2)ccc1Br]

1-(4-Bromo-3-methylphenyl)-4-(4-boronophenyl)-1H-1,2,3-triazole was prepared from Z)—N′-(1-(4-bromophenyl)ethylidene)benzene-sulfono hydrazide and 4-bromo-3-methylaniline. (Z)—N-(1-(4-Bromophenyl)ethylidene)-benzene sulfonohydrazide was made from 1-(4-bromophenyl)ethanone and benzenesulfono hydrazide.

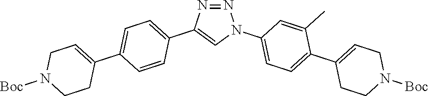 [SMILES: Cc1cc(-n2cc(-c3ccc(C4=CCN(C(=O)OC(C)(C)C)CC4)cc3)nn2)ccc1C1=CCN(C(=O)OC(C)(C)C)CC1]

4-(4-(1-(3-Methyl-4-(1,2,3,6-tetrahydropyridin-4-yl)phenyl)-1H-1,2,3-triazol-4-yl)phenyl)-1,2,3,6-tetrahydropyridine was prepared from 1-(4-bromo-3-methylphenyl)-4-(4-bromophenyl)-1H-1,2,3-triazole and tert-butyl 4-(4,4,5,5-tetramethyl-1,3,2-dioxaborolan-2-yl)-5,6-dihydropyridine-1(2H)-carboxylate.

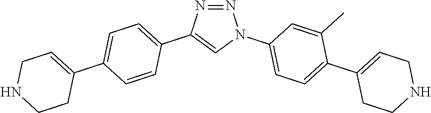 [SMILES: Cc1cc(-n2cc(-c3ccc(C4=CCNCC4)cc3)nn2)ccc1C1=CCNCC1]

4-(4-(1-(3-Methyl-4-(1,2,3,6-tetrahydropyridin-4-yl)phenyl)-1H-1,2,3-triazol-4-yl)phenyl)-1,2,3,6-tetrahydropyridine was prepared from 4-(4-(1-(3-methyl-4-(1,2,3,6-tetrahydropyridin-4-yl)phenyl)-1H-1,2,3-triazol-4-yl)phenyl)-1,2,3,6-tetrahydropyridine

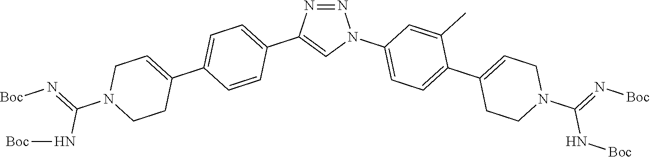 [SMILES: Cc1cc(-n2cc(-c3ccc(C4=CCN(/C(=N/C(=O)OC(C)(C)C)NC(=O)OC(C)(C)C)CC4)cc3)nn2)ccc1C1=CCN(/C(=N/C(=O)OC(C)(C)C)NC(=O)OC(C)(C)C)CC1]

tert-Butyl N—[(Z)-(4-{4-[4-(4-{1-[(E)-{[(tert-butoxy)carbonyl]amino}({[(tert-butoxy)carbonyl]imino})methyl]-1,2,3,6-tetrahydropyridin-4-yl}phenyl)-1H-1,2,3-triazol-1-yl]-2-methylphenyl}-1,2,3,6-tetrahydropyridin-1-yl)({[(tert-butoxy)carbonyl]imino})methyl] carbamate was prepared from 4-(4-(1-(3-methyl-4-(1,2,3,6-tetrahydropyridin-4-yl)phenyl)-1H-1,2,3-triazol-4-yl)phenyl)-2,3,6-tetrahydropyridine.

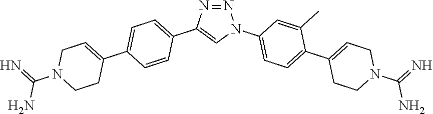 [SMILES: Cc1cc(-n2cc(-c3ccc(C4=CCN(C(=N)N)CC4)cc3)nn2)ccc1C1=CCN(C(=N)N)CC1]

### Example 35

4-(4-(1-(4-(1-Carbamimidoyl-1,2,3,6-tetrahydropyridin-4-yl)-3-methyl phenyl)-1H-1,2,3-triazol-4-yl)phenyl)-5,6-dihydropyridine-1(2H)-carboximidamide was prepared from tert-butyl N—[(Z)-(4-{4-[4-(4-{1-[(E)-{[(tert-butoxy)carbonyl]amino}({[(tert-butoxy)carbonyl] imino})methyl]-1,2,3,6-tetrahydropyridin-4-yl}phenyl)-1H-1,2,3-triazol-1-yl]-2-methylphenyl}-1,2,3,6-tetrahydropyridin-1-yl)({[(tert-butoxy)carbonyl]imino})methyl]carbamate. ¹H NMR (300 MHz, METHANOL-d₄) δ=8.92 (s, 1H), 7.93-7.65 (m, 2H), 7.57 (br d, J=8.4 Hz, 2H), 7.35 (br d, J=8.3 Hz, 3H), 6.24 (br s, 1H), 5.75 (br s, 1H), 4.14 (br dd, J=3.3, 6.5 Hz, 4H), 3.72 (br dd, J=4.6, 6.1 Hz, 4H), 2.74 (br s, 2H), 2.57 (br s, 2H), 2.44 (s, 3H). LC/MS method A: R_{f}=3.10 mins., (M+H)⁺=482, purity >95%.

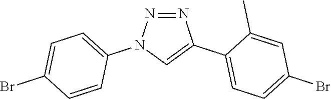 [SMILES: Cc1cc(Br)ccc1-c1cn(-c2ccc(Br)cc2)nn1]

4-(4-Bromo-2-methylphenyl)-1-(4-bromophenyl)-1H-1,2,3-triazole was prepared from (Z)—N′-(1-(4-bromo-2-methylphenyl)ethylidene)-benzenesulfonohydrazide and 4-bromoaniline. (Z)—N′-(1-(4-Bromophenyl)ethylidene)-benzenesulfonohydrazide was prepared from 1-(4-bromophenyl)ethanone and benzenesulfonohydrazide.

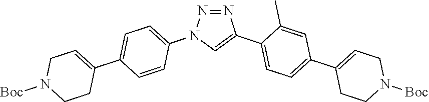 [SMILES: Cc1cc(C2=CCN(C(=O)OC(C)(C)C)CC2)ccc1-c1cn(-c2ccc(C3=CCN(C(=O)OC(C)(C)C)CC3)cc2)nn1]

tert-Butyl 4-(4-(4-(4-(1-(tert-butoxycarbonyl)-1,2,3,6-tetrahydropyridin-4-yl)-2-methylphenyl)-1H-1,2,3-triazol-1-yl)phenyl)-5,6-dihydropyridine-1(2H)-carboxylate was prepared from 4-(4-bromo-2-methylphenyl)-1-(4-bromophenyl)-1H-1,2,3-triazole and tert-butyl 4-(4,4,5,5-tetramethyl-1,3,2-dioxaborolan-2-yl)-5,6-dihydropyridine-1(2H)-carboxylate.

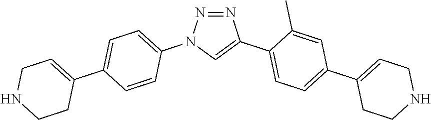 [SMILES: Cc1cc(C2=CCNCC2)ccc1-c1cn(-c2ccc(C3=CCNCC3)cc2)nn1]

4-(4-(4-(2-Methyl-4-(1,2,3,6-tetrahydropyridin-4-yl)phenyl)-1H-1,2,3-triazol-1-yl)phenyl)-1,2,3,6-tetrahydropyridine was prepared from tert-butyl 4-(4-(4-(4-(1-(tert-butoxycarbonyl)-1,2,3,6-tetrahydropyridin-4-yl)-2-methylphenyl)-1H-1,2,3-triazol-1-yl)phenyl)-5,6-dihydropyridine-1(2H)-carboxylate.

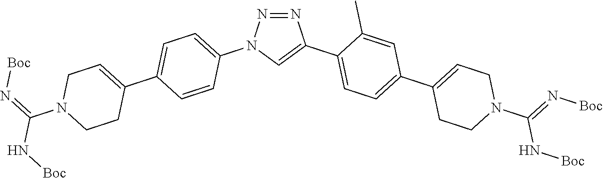 [SMILES: Cc1cc(C2=CCN(/C(=N/C(=O)OC(C)(C)C)NC(=O)OC(C)(C)C)CC2)ccc1-c1cn(-c2ccc(C3=CCN(/C(=N\C(=O)OC(C)(C)C)NC(=O)OC(C)(C)C)CC3)cc2)nn1]

tert-Butyl N-[(4-{4-[4-(4-{1-[(E)-{[(tert-butoxy)carbonyl]amino}({[(tert-butoxy)carbonyl]imino})methyl]-1,2,3,6-tetrahydropyridin-4-yl}-2-methylphenyl)-1H-1,2,3-triazol-1-yl]phenyl}-1,2,3,6-tetrahydropyridin-1-yl)({[(tert-butoxy)carbonyl]imino})methyl] carbamate was prepared from 4-(4-(4-(2-methyl-4-(1,2,3,6-tetrahydropyridin-4-yl)phenyl)-1H-1,2,3-triazol-1-yl)phenyl)-1,2,3,6-tetrahydropyridine.

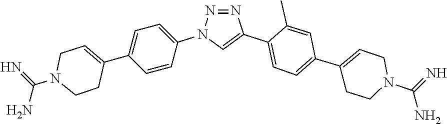 [SMILES: CCC/C(=C\CNC(=N)N)c1ccc(-c2cn(-c3ccc(C4=CCN(C(=N)N)CC4)cc3)nn2)c(C)c1]

### Example 36

4-(4-(4-(4-(1-Carbamimidoyl-1,2,3,6-tetrahydropyridin-4-yl)-2-methylphenyl)-1H-1,2,3-triazol-1-yl)phenyl)-5,6-dihydropyridine-1(2H)-carboximidamide was prepared from tert-butyl N-[(4-{4-[4-(4-{1-[(E)-{[(tert-butoxy)carbonyl]amino}({[(tert-butoxy) carbonyl]imino})methyl]-1,2,3,6-tetrahydropyridin-4-yl}-2-methylphenyl)-1H-1,2,3-triazol-1-yl] phenyl}-1,2,3,6-tetrahydropyridin-1-yl)({[(tert-butoxy)carbonyl]imino})methyl] carbamate. ¹H NMR (300 MHz, METHANOL-d₄) δ=8.65 (s, 1H), 7.87 (d, J=8.7 Hz, 2H), 7.75-7.55 (m, 3H), 7.44-7.27 (m, 2H), 6.17 (br d, J=19.2 Hz, 2H), 4.07 (dd, J=3.3, 6.5 Hz, 4H), 3.64 (q, J=5.7 Hz, 4H), 2.66 (br d, J=7.4 Hz, 4H), 2.46 (s, 3H). LC/MS method A: R_{f}=3.09 mins., (M+H)⁺==286,482, purity >95%.

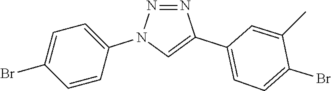 [SMILES: Cc1cc(-c2cn(-c3ccc(Br)cc3)nn2)ccc1Br]

4-(4-Bromo-3-methylphenyl)-1-(4-bromophenyl)-1H-1,2,3-triazole was prepared from (Z)—N′-(1-(4-bromo-3-methylphenyl)ethylidene)-benzenesulfonohydrazide and 4-bromoaniline. (Z)—N′-(1-(4-Bromophenyl)ethylidene)-benzenesulfonohydrazide was made from 1-(4-bromophenyl)ethanone and benzenesulfonohydrazide.

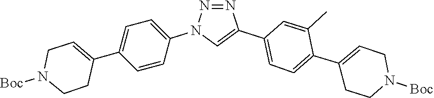 [SMILES: Cc1cc(-c2cn(-c3ccc(C4=CCN(C(=O)OC(C)(C)C)CC4)cc3)nn2)ccc1C1=CCN(C(=O)OC(C)(C)C)CC1]

tert-Butyl 4-(4-(4-(4-(1-(tert-butoxycarbonyl)-1,2,3,6-tetrahydropyridin-4-yl)-3-methylphenyl)-1H-1,2,3-triazol-1-yl)phenyl)-5,6-dihydropyridine-1(2H)-carboxylate was prepared from 4-(4-bromo-3-methylphenyl)-1-(4-bromophenyl)-1H-1,2,3-triazole and tert-butyl 4-(4,4,5,5-tetramethyl-1,3,2-dioxaborolan-2-yl)-5,6-dihydropyridine-1(2H)-carboxylate.

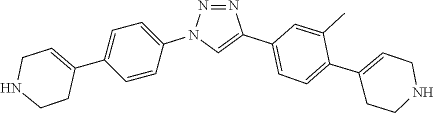 [SMILES: CCC/C(=C\CN)c1ccc(-c2cn(-c3ccc(C4=CCNCC4)cc3)nn2)cc1C]

4-(4-(4-(3-Methyl-4-(1,2,3,6-tetrahydropyridin-4-yl)phenyl)-1H-1,2,3-triazol-1-yl)phenyl)-1,2,3,6-tetrahydropyridine was prepared from tert-butyl 4-(4-(4-(4-(1-(tert-butoxycarbonyl)-1,2,3,6-tetrahydropyridin-4-yl)-3-methylphenyl)-1H-1,2,3-triazol-1-yl) phenyl)-5,6-dihydropyridine-1(2H)-carboxylate.

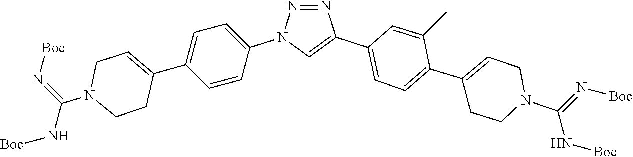 [SMILES: CCC/C(=C\CN/C(=N/C(=O)OC(C)(C)C)NC(=O)OC(C)(C)C)c1ccc(-c2cn(-c3ccc(C4=CCN(/C(=N\C(=O)OC(C)(C)C)NC(=O)OC(C)(C)C)CC4)cc3)nn2)cc1C]

tert-Butyl N-[4(4-{4-[4-(4-{1-[(E)-{[(tert-butoxy)carbonyl]amino}({[(tert-butoxy)carbonyl]imino})methyl]-1,2,3,6-tetrahydropyridin-4-yl}-3-methylphenyl)-1H-1,2,3-triazol-1-yl]phenyl}-1,2,3,6-tetrahydropyridin-1-yl)({[(tert-butoxy)carbonyl]imino}) methyl]carbamate was prepared from 4-(4-(4-(3-methyl-4-(1,2,3,6-tetrahydropyridin-4-yl)phenyl)-1H-1,2,3-triazol-1-yl)phenyl)-1,2,3,6-tetrahydropyridine.

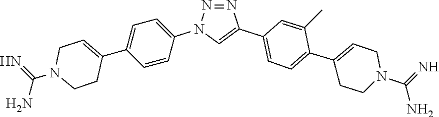 [SMILES: CCC/C(=C\CNC(=N)N)c1ccc(-c2cn(-c3ccc(C4=CCN(C(=N)N)CC4)cc3)nn2)cc1C]

### Example 37

4-(4-(4-(4-(1-Carbamimidoyl-1,2,3,6-tetrahydropyridin-4-yl)-3-methyl phenyl)-1H-1,2,3-triazol-1-yl)phenyl)-5,6-dihydropyridine-1(2H)-carboximidamide was prepared from tert-butyl N-[(4-{4-[4-(4-{1-[(E)-{[(tert-butoxy)carbonyl]amino}({[(tert-butoxy)carbonyl] imino})methyl]-1,2,3,6-tetrahydropyridin-4-yl}-3-methylphenyl)-1H-1,2,3-triazol-1-yl]phenyl}-1,2,3,6-tetrahydropyridin-1-yl)({[(tert-butoxy)carbonyl]imino})methyl]carbamate. ¹H NMR. (300 MHz, CD₃OD) δ=8.95 (s, 1H), 8.08-7.86 (m, 3H), 7.86-7.59 (m, 3H), 7.23 (d, J=7.9 Hz, 2H), 6.33-6.21 (m, 1H), 5.85-5.65 (m, 1H), 4.31 (s, 1H), 4.25-3.97 (m, 4H), 3.81-3.49 (m, 4H), 2.65-2.47 (m, 4H), 2.35 (s, 1H). LC/MS method A: R_{f}=3.10 mins., (M+H)⁺=482, purity >95%.

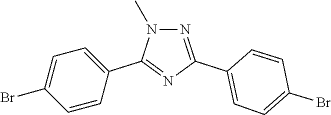 [SMILES: Cn1nc(-c2ccc(Br)cc2)nc1-c1ccc(Br)cc1]

Synthesis of 3,5-Bis(4-bromophenyl)-1-methyl-1H-1,2,4-triazole A flask was charged with 1090 mg (5.43 mmol) of 4-bromobenzoic acid, 8 mL of dry N, N-dimethylformamide and 2100 mg (16.3 mmol) of N,N-diisopropylethylaminde. After five minutes of stirring, 2470 mg (6.51 mmol) of (1-[Bis(dimethylamino)methylene]-1H-1,2,3-triazolo[4,5-b]pyridinium 3-oxide hexafluorophosphate added at once and stirred for 40 minutes. Then 0.1280 mg (5.43 mmol) of 4-bromobenzimidamide HCl was added at once and reaction stirred for 18 hours. The reaction was then diluted with 30 mL ethyl acetate and washed with three 20 mL portions of water, dried and concentrated under vacuum. The reaction was dissolved with 4 mL of glacial acetic acid and 315 mg (7.5 mmol) of methylhydrazine was added. The reaction was heated at 80° C. for 18 hours. The reaction cooled and diluted with 10 mL of water. Solid collected, rinsed with 5 mL of water and air dried in hood to yield 1.63 grams (76%).

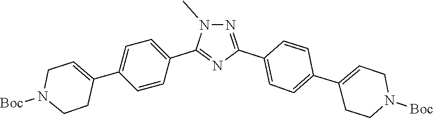 [SMILES: Cn1nc(-c2ccc(C3=CCN(C(=O)OC(C)(C)C)CC3)cc2)nc1-c1ccc(C2=CCN(C(=O)OC(C)(C)C)CC2)cc1]

Synthesis of tert-Butyl 4,4′-(4,4′-(1-methyl-1H-1,2,4-triazole-3,5-diyl)bis(4,1-phenylene))bis(5,6-dihydropyridine-1(2H)-carboxylate): A flask was charged with 235 mg (0.598 mmol) of 3,5-bis(4-bromophenyl)-1-methyl-1H-1,2,4-triazole, 11 mg (0.048 mmol) of palladium acetate, 44 mg (0.108 mmol) of 2-dicyclohexylphosphino-2′,6′-dimethoxy biphenyl, 462 mg (1.53 mmol) of tert-butyl 4-(4,4,5,5-tetramethyl-1,3,2-dioxaborolan-2-yl)-5,6-dihydropyridine-1(2H)-carboxylate, 6 mL of 1,4-dioxane and 2.25 mL of 2.0 molar potassium carbonate aqueous solution. The flask was swept with nitrogen for eight minutes and heated at 95° C. for 18 hours. The reaction was complete by LCMS and the reaction was allowed to cool and diluted with 30 mL water and 40 mL of ethyl acetate added over five minutes. Organic phase was dried and concentrated under vacuum. Concentrate was dissolved with dichloromethane and chromatographed on silica using a step gradient using (15% isopropanol/dichloromethane)/dichloromethane from 0 to 100%. Similar fractions combined and concentrated under vacuum to yield 360 mg in quantitative yield.

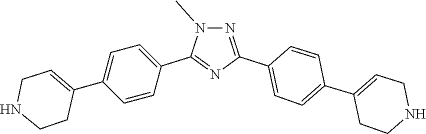 [SMILES: Cn1nc(-c2ccc(C3=CCNCC3)cc2)nc1-c1ccc(C2=CCNCC2)cc1]

Synthesis of 4,4′-(4,4′-(1-Methyl-1H-1,2,4-triazole-3,5-diyl)bis(4,1-phenylene))bis(1,2,3,6-tetrahydropyridine): A flask was charged with 360 mg (0.598 mmol) of the tert-butyl 4,4-(4,4-(1-methyl-1H-1,2,4-triazole-3,5-diyl)bis(4,1-phenylene))bis(5,6-dihydropyridine-1(2H)-carboxylate) which was slurried with 3 mL of dichloromethane. Then 5 mL (65 mmol) of trifluoroacetic acid added over two minutes with stirring. Three hours later hour the reaction was concentrated under vacuum and triturated with two 30 mL portions of ether and ether decanted away to yield a solid in quantitative yield.

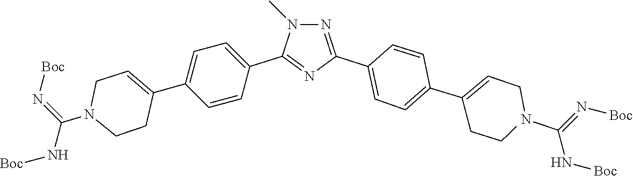 [SMILES: CCC/C(=C\CN/C(=N/C(=O)OC(C)(C)C)NC(=O)OC(C)(C)C)c1ccc(-c2nc(-c3ccc(C4=CCN(/C(=N\C(=O)OC(C)(C)C)NC(=O)OC(C)(C)C)CC4)cc3)n(C)n2)cc1]

Synthesis of tert-Butyl 4′-(4,4′-(1-methyl-1H-1,2,4-triazole-3,5-diyl)bis(4,1-phenylene)) bis(5,6-dihydropyridine-4,1(2H)-diyl))bis(methane-1-yl-1,1-diylidene) tetra carbamate) A flask was charged with 334 mg (0.50 mmol) of 4,4′-(4,4′-(1-methyl-1H-1,2,4-triazole-3,5-diyl)bis(4,1-phenylene))bis(1,2,3,6-tetrahydropyridine) bis trifluoroacetic acid salt, 3 mL N,N-dimethylformamide and 4 mL dichloromethane. Then 560 mg (5.54 mmol) of triethylamine added over two minutes. Then 420 mg (1.35 mmol) of tert-butyl (1H-pyrazol-1-yl)methanediylidenedicarbamate was added over several minutes. The reaction slurry was stirred at 23° C. for 18 hours. Then 150 mg (0.483 mmol) of tert-butyl (1H-pyrazol-1-yl) methanediylidenedicarbamate was added. After 24 hours, the reaction was diluted with 50 mL, ethyl acetate and then washed with three portions of 40 mL of water. Organic phase was dried with sodium sulfate and concentrated under vacuum. Purified by normal phase chromatography, loaded as dichloromethane solution, eluted with (15% isopropanol/dichloromethane)/dichloromethane with step gradient from 0 to 100% to elute product. Yield was 370 mg of product (84%).

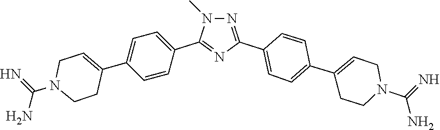 [SMILES: CCC/C(=C\CNC(=N)N)c1ccc(-c2nc(-c3ccc(C4=CCN(C(=N)N)CC4)cc3)n(C)n2)cc1]

### Example 38

Synthesis of 4,4′-(4,4′-(1-Methyl-1H-1,2,4-triazole-3,5-diyl)bis(4,1-phenylene)) bis(5,6-dihydropyridine-1(2H)-carboximidamide): A flask was charged with 370 mg (0.420 mmol) of tert-butyl (4,4′-(4,4′-(1-methyl-1H-1,2,4-triazole-3,5-diyl)bis(4,1-phenylene))bis(5,6-dihydropyridine-4,1(2H)-diyl))bis(methane-1-yl-1,1-diylidene)tetracarbamate) which was dissolved with 3 mL of dichloromethane. Then 5 mL (65 mmol) of trifluoroacetic acid added over two minutes with stirring and stirred for 18 hours. The LCMS indicted reaction complete and reaction concentrated under vacuum. Oil was triturated with two 20 mL portions of ethyl acetate and solvent decanted away to yield a solid. Solid was dissolved in N, N-dimethylformamide and purified by prep HPLC. Yield was 80 mg (27%). ¹H NMR (300 MHz, CD₃OD) δ=8.07 (d, J=8.7 Hz, 2H), 7.94-7.76 (m, 2H), 7.75-7.64 (m, 2H), 7.63-7.42 (m, 2H), 6.39-6.08 (m, 2H), (in, 1H), 4.45-4.10 (m, 2H), 4.04 (s, 3H), 3.81-3.65 (m, 4H), 2.92-2.61 (m, 4H). LC/MS method A: R_{f}=3.10 mins., (M+H)⁺=482, purity >95%.

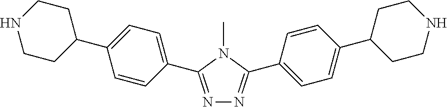 [SMILES: Cn1c(-c2ccc(C3CCNCC3)cc2)nnc1-c1ccc(C2CCNCC2)cc1]

Synthesis of 4-(4-{4-Methyl-5-[4-(piperidin-4-yl)phenyl]-4H-1,2,4-triazol-3-yl}phenyl)piperidine: A solution of 4-(4-{4-methyl-5-[4-(1,2,3,6-tetrahydropyridin-4-yl) phenyl]-4H-1,2,4-triazol-3-yl}phenyl)-1,2,3,6-tetrahydropyridine (26 mg, (38 mop in methanol (3 mL) was hydrogenated over 10% palladium on carbon (10 mg) at 45 psi initial hydrogen pressure for 24 hours. The catalyst was filtered and the solvent was evaporated to leave a clear thick gum (28 mg, 100%). 1H-NMR (CD₃OD) δ 7.77 (d, 4H, J=8.5 Hz), 7.54 (d, 4H, J=8.5 Hz), 3.75 (s, 3H), 3.47-3.60 (m, 4H), 3.00-3.25 (m, 7H), 1.90-2.10 (m, 7H). LCMS method A R_{f}=2.14 mins, purity >95%, (M+H)⁺=402.

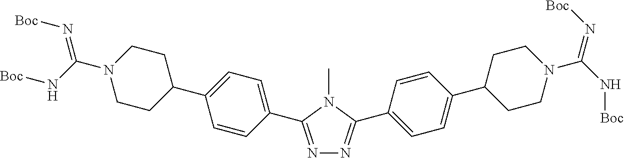 [SMILES: Cn1c(-c2ccc(C3CCN(/C(=N\C(=O)OC(C)(C)C)NC(=O)OC(C)(C)C)CC3)cc2)nnc1-c1ccc(C2CCN(/C(=N/C(=O)OC(C)(C)C)NC(=O)OC(C)(C)C)CC2)cc1]

tert-Butyl. (4,4′-(4,4′-(4-methyl-4H-1,2,4-triazole-3,5-diyl)bis(4,1-phenylene)) bis(piperidine-4,1-diyl))bis(methanetriyl)tetracarbamate was prepared from 4,4′-(4,4′-(4-methyl-4H-1,2,4-triazole-3,5-diyl)bis(4,1-phenylene))dipiperidine.

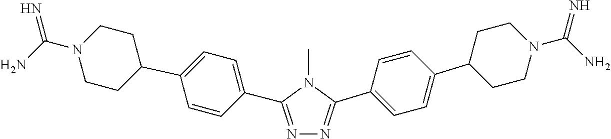 [SMILES: Cn1c(-c2ccc(C3CCN(C(=N)N)CC3)cc2)nnc1-c1ccc(C2CCN(C(=N)N)CC2)cc1]

### Example 39

Synthesis of 4,4′-(4,4′-(4-Methyl-4H-1,2,4-triazole-3,5-diyl)bis(4,1-phenylene))dipiperidine-1-carboximidamide: A solution of 4-(4-{4-methyl-5-[4-(piperidin-4-yl) phenyl]-4H-1,2,4-triazol-3-yl}phenyl)piperidine (23 mg, 31 μmol), N—((N′,N″-bis-tert-butyloxy carbonyl)amidino)pyrrazole (29 mg, 93 μmol) and N,N-diisopropyl ethylamine (32 mg, 248 μmol, 44 μl) in methanol (0.5 mL) was stirred for 24 hours and evaporated to dryness. The crude product was dissolved in dichloromethane (1 mL) and trifluoroacetic acid (1 mL) and stirred for 3 h. The solvents were evaporated and the crude product was purified by preparative reverse phase and the product fractions were combined and lyophilized to leave a white powder (12 mg, 47%). ¹H NMR. (300 MHz, CD₃OD-d₄) δ=7.75 (d, J=8.2 Hz, 4H), 7.54 (d, J=8.2 Hz, 4H), 4.20-4.00 (m, 2H), 3.76 (s, 3H), 3.24 (dd, J=6.2, 7.0 Hz, 2H), 2.08-1.97 (m, 2H), 1.82 (br dd, J=3.3, 12.4 Hz, 2H). LC/MS method A: R_{f}=2.52 mins., (M+H)⁺==486, purity >95%.

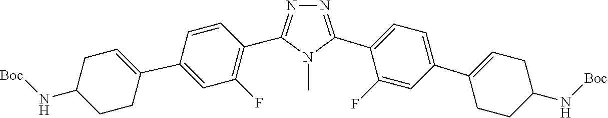 [SMILES: Cn1c(-c2ccc(C3=CCC(NC(=O)OC(C)(C)C)CC3)cc2F)nnc1-c1ccc(C2=CCC(NC(=O)OC(C)(C)C)CC2)cc1F]

Synthesis of tert-Butyl 4,4′-(4,4-(4-methyl-4H-1,2,4-triazole-3,5-diyl)bis(3-fluoro-4,1-phenylene))bis(cyclohex-3-ene-4,1-diyl)dicarbamate: A flask was charged with 370 mg (0.9 mmol) of 3,5-Bis(4-bromo-2-fluorophenyl)-4-methyl-4H-1,2,4-triazole, 16 mg (0.072 mmol) of palladium acetate, 74 mg (0.18 mmol) of 2-dicyclohexylphosphino-2′,6′-dimethoxybiphenyl, 758 mg (2.34 mmol) of tert-butyl 4-(4,4,5,5-tetramethyl-1,3,2-dioxaborolan-2-yl)cyclohex-3-enylcarbamate, 7 mL of 1,4-dioxane and 3.4 mL of 2.0 molar potassium carbonate aqueous solution. The flask was swept with nitrogen for five minutes and heated at 95° C. for 18 hours. The reaction was complete by LCMS and the reaction was allowed to cool and diluted with 5 mL water and 10 mL of ethyl acetate added over two minutes. Solids formed quickly and reaction stirred for one hour. Solid collected on a filter and rinsed with water and ethyl acetate and air dried for 90 minutes. Yield was 300 mg (49%).

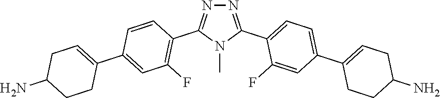 [SMILES: Cn1c(-c2ccc(C3=CCC(N)CC3)cc2F)nnc1-c1ccc(C2=CCC(N)CC2)cc1F]

Synthesis of 4,4′-(4,4′-(4-Methyl-4H-1,2,4-triazole-3,5-diyl)bis(3-fluoro-4,1-phenylene)) dicyclohex-3-enamine: A flask was charged with 300 mg (0.488 mmol) of tert-butyl 4,4′-(4,4′-(4-methyl-4H-1,2,4-triazole-3,5-diyl)bis(3-fluoro-4,1-phenylene))bis (cyclohex-3-ene-4,1-diyl)dicarbamate was slurried with 400 mg (0.70 mmol) of anisole and 7 mL dichloromethane. Then 8 mL (104 mmol) of trifluoroacetic acid was added over five minutes with stirring. After two hours the reaction was concentrated under vacuum and triturated with 15 mL of ether and ether decanted away to yield a solid in quantitative yield.

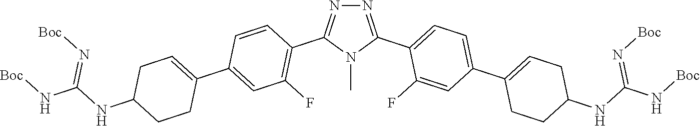 [SMILES: Cn1c(-c2ccc(C3=CCC(N/C(=N/C(=O)OC(C)(C)C)NC(=O)OC(C)(C)C)CC3)cc2F)nnc1-c1ccc(C2=CCC(N/C(=N/C(=O)OC(C)(C)C)NC(=O)OC(C)(C)C)CC2)cc1F]

Synthesis of tert-Butyl (4,4′-(4,4′-(4-methyl-4H-1,2,4-triazole-3,5-diyl)bis(3-fluoro-4,1-phenylene))bis(cyclohex-3-ene-4,1-diyl))bis(azanediyl)bis((tert-butoxycarbonyl amino)-methan-1-yl-1-ylidene)dicarbamate: A flask was charged with 560 mg (0.75 mmol) of 4,4′-(4,4′-(4-methyl-4H-1,2,4-triazole-3,5-diyl)bis(3-fluoro-4,1-phenylene))dicyclohex-3-enamine bis trifluoroacetic acid salt and 3 mL N,N-dimethylformamide. Then 530 mg (5.05 mmol) of triethylamine was added over two minutes. Then 581 mg (1.88 mmol) of tert-butyl (1H-pyrazol-1-yl)methanediylidenedicarbamate was added. The reaction was stirred for 18 hours at room temperature. The reaction was diluted with 40 mL ethyl acetate and then washed with three portions of 30 mL of water. Organic phase was dried with sodium sulfate and concentrated under vacuum. Purified by normal phase chromatography, loaded as dichloromethane solution, eluted with ethyl acetate/dichloromethane with step gradient from 0 to 75% to elute product. Yield was 370 mg of product (52%).

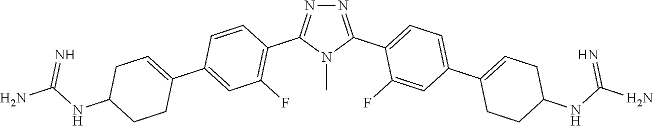 [SMILES: Cn1c(-c2ccc(C3=CCC(NC(=N)N)CC3)cc2F)nnc1-c1ccc(C2=CCC(NC(=N)N)CC2)cc1F]

### Example 40

Synthesis of 1-[4-(4-{5-[4-(4-Carbamimidamidocyclohex-1-en-1-yl)-2-fluorophenyl]-4-methyl-4H-1,2,4-triazol-3-yl}-3-fluorophenyl)cyclohex-3-en-1-yl]guanidine: A flask was charged with 370 mg (0.381 mmol) of tert-butyl (4,4′-(4,4′-(4-methyl-4H-1,2,4-triazole-3,5-diyl)bis(3-fluoro-4,1-phenylene))bis(cyclohex-3-ene-4,1-diyl))bis(azanediyl)bis((tert-butoxycarbonyl amino)-methan-1-yl-1-ylidene)dicarbamate which was dissolved with a mixture of 250 mg (2.31 mmol) of anisole and 3 mL of dichloromethane. Then 7 mL (90.9 mmol) of trifluoroacetic acid added over two minutes with stirring and stirred for 18 hours. The LCMS indicted reaction complete and reaction concentrated under vacuum. Oil was triturated with two 30 mL portions of ether and ether decanted away to yield a solid. Solid was dissolved in N,N-dimethylformamide and was purified by prep HPLC. Yield was 128 mg (43%). ¹H NMR (300 MHz, DMSO-d₆) δ=7.71-7.47 (m, 6H), 7.27-6.93 (br s, 6H), 6.38 (br s, 2H), 3.74 (s, 3H), 3.49-3.43 (m, 4H), 2.63-2.50 (m, 4H), 2.26-2.12 (m, 2H), 1.98 (br d, J=8.5 Hz, 2H), 1.71 (br dd, J=7.5, 14.8 Hz, 2H). LC/MS method A: R_{f}=3.23 mins., (M+H)⁺=661,546, purity >90%.

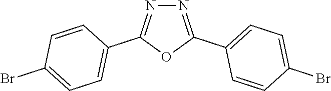 [SMILES: Brc1ccc(-c2nnc(-c3ccc(Br)cc3)o2)cc1]

2,5-Bis(4-bromophenyl)-1,3,4-oxadiazole was prepared from (4-Bromo-benzoic acid N′-(4-bromo-benzoyl)-hydrazide and phosphorus oxychloride.

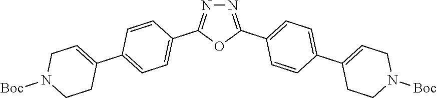 [SMILES: CC(C)(C)OC(=O)N1CC=C(c2ccc(-c3nnc(-c4ccc(C5=CCN(C(=O)OC(C)(C)C)CC5)cc4)o3)cc2)CC1]

(tert-Butyl 4,4′-(4,4′-(1,3,4-oxadiazole-2,5-diyl)bis(4,1-phenylene))bis(5,6-dihydropyridine-1(2H)-carboxylate) was prepared from 2,5-bis(4-bromophenyl)-1,3,4-oxadiazole and tert-butyl 4-(4,4,5,5-tetramethyl-1,3,2-dioxaborolan-2-yl)-5,6-dihydro pyridine-1(2H)-carboxylate.

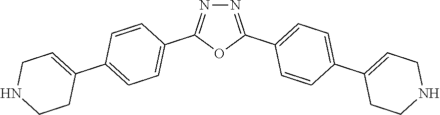 [SMILES: C1=C(c2ccc(-c3nnc(-c4ccc(C5=CCNCC5)cc4)o3)cc2)CCNC1]

2,5-Bis(4-(1,2,3,6-tetrahydropyridin-4-yl)phenyl)-1,3,4-oxadiazole was prepared from tert-butyl 4,4-(4,4-(1,3,4-oxadiazole-2,5-diyl)bis(4,1-phenylene))bis(5,6-dihydropyridine-1(2H)-carboxylate).

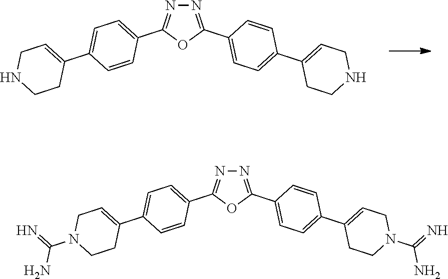 [SMILES: C1=C(c2ccc(-c3nnc(-c4ccc(C5=CCNCC5)cc4)o3)cc2)CCNC1 ; N=C(N)N1CC=C(c2ccc(-c3nnc(-c4ccc(C5=CCN(C(=N)N)CC5)cc4)o3)cc2)CC1]

### Example 41

Synthesis of 4-(4-{5-[4-(1-Carbamimidoyl-1,2,3,6-tetrahydropyridin-4-yl)phenyl]-1,3,4-oxadiazol-2-yl}phenyl)-1,2,3,6-tetrahydropyridine-1-carboximidamide: A solution of 4-(4-{5-[4-(1,2,3,6tetrahydropyridin-4-yl)phenyl]-1,3,4-oxadiazol-2-yl}phenyl)-1,2,3,6-tetrahydropyridine (30 mg, 49 μmol) and triethylamine (39 mg, 0.39 mmol, 54 μl) in methanol (0.5 mL) was treated with N—((N′,N″-bis-tert-butyloxycarbonyl) amidino)pyrrazole (45 mg, 0.15 mmol) and stirred 18 hours. N,N-Dimethylformamide (1 mL) was added for solubilization and the mixture was purified by reverse phase HPLC The product fractions were combined, treated with saturated sodium bicarbonate solution and extracted with dichloromethane (2×25 mL). The combined organic layers were dried (Na₂SO₄) and evaporated to a glassy solid which was dissolved in trifluoroacetic acid (1 mL) and dichloromethane (1 mL) and stirred for 2 hours. The solvents were evaporated and the product was lyophilized to a white solid (23 mg, 67%). ¹H-NMR (CD₃OD) δ 8.16 (d, 4H, J=8.5 Hz), 7.72 (d, 4H, J=8.5 Hz), 6.35 (m, 2H), 4.18 (m, 4H), 3.72 (m, 4H), 2.75 (m, 4H). LCMS method A R_{f}=3.33 mins, purity >95%, (M+H)⁺=469

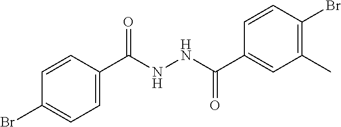 [SMILES: Cc1cc(C(=O)NNC(=O)c2ccc(Br)cc2)ccc1Br]

4-Bromo-N′-(4-bromobenzoyl)-3-methylbenzohydrazid was prepared from 4-bromo-3-methylbenzoyl chloride and 4-bromo-benzoic acid hydrazide.

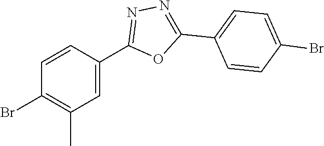 [SMILES: Cc1cc(-c2nnc(-c3ccc(Br)cc3)o2)ccc1Br]

2-(4-Bromo-3-methylphenyl)-5-(4-bromophenyl)-1,3,4-oxadiazole was prepared from 4-bromo-N′-(4-bromobenzoyl)-3-methylbenzohydrazide and phosphorus oxychloride.

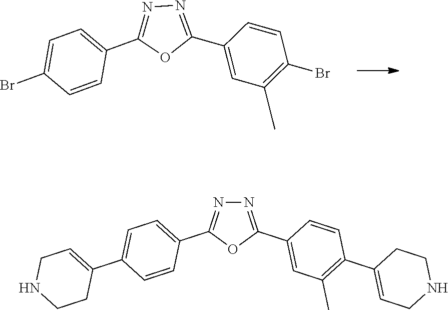 [SMILES: Cc1cc(-c2nnc(-c3ccc(Br)cc3)o2)ccc1Br ; Cc1cc(-c2nnc(-c3ccc(C4=CCNCC4)cc3)o2)ccc1C1=CCNCC1]

Synthesis of 4-(4-{5-[3-Methyl-4-(1,2,3,6-tetrahydropyridin-4-yl)phenyl]-1,3,4-oxadiazol-2-yl}phenyl)-1,2,3,6-tetrahydropyridine: A mixture 2-(4-bromo-3-methyl phenyl)-5-(4-bromophenyl)-1,3,4-oxadiazole (100 mg, 0.25 mmol), tert-butyl 4-(4,4,5,5-tetramethyl-1,3,2-dioxaborolan-2-yl)-1,2,3,6-tetrahydropyridine-1-carboxylate (232 mg, 0.75 mmol), dicyclohexyl(2′,6′-dimethoxybiphenyl-2-yl) phosphine (16 mg, 18 mop, palladium acetate (4.0 mg, 18 μmol), potassium carbonate (260 mg, 0.1.9 mmol), water (0.8 mL) and 1,4-dioxane (2 mL) was purged with nitrogen, stirred and heated to 90° C. for 20 hours. The mixture was diluted with dichloromethane (20 mL) and water (20 mL) and the aqueous layer was extracted with dichloromethane (20 mL). The combined dichloromethane layers were dried (Na₂SO₄) and evaporated. The crude product mixture was dissolved in trifluoroacetic acid (1 mL) and dichloromethane (1 mL) and stirred for 2 hours. The mixture was evaporated, purified by preparative reverse phase HPLC and the product fractions were lyophilized to leave the product as a white solid (bis trifluoroacetic acid salt, 111 mg, 60%). ¹H-NMR (CD₃OD) δ 8.18 (d, 2H, J=8.5 Hz), 8.04 (s, 1H), 7.99 (d, 1H, J=8.2 Hz), 7.74 (d, 2H, J=8.2 Hz), 7.38 (d, 1H, J=8.0 Hz), 6.37 (m, 1H), 5.75 (m, 1H), 3.89 (m, 4H), 3.51 (m, 4H), 2.85 (m, 2H), 2.65 (m, 2H), 2.44 (s, 3H). LCMS method A R_{f}=2.66 mins, purity >95%, (M+H)⁺=399.

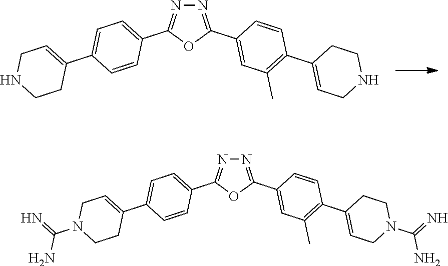 [SMILES: Cc1cc(-c2nnc(-c3ccc(C4=CCNCC4)cc3)o2)ccc1C1=CCNCC1 ; Cc1cc(-c2nnc(-c3ccc(C4=CCN(C(=N)N)CC4)cc3)o2)ccc1C1=CCN(C(=N)N)CC1]

### Example 42

4-(4-(5-(4-(1-Carbamimidoyl-1,2,3,6-tetrahydropyridin-4-yl)-3-methyl phenyl)-1,3,4-oxadiazol-2-yl)phenyl)-5,6-dihydropyridine-1(2H)-carboximidamide A solution of 4-(4-{5-[3-methyl-4-(1,2,3,6-tetrahydropyridin-4-yl)phenyl]-1,3,4-oxadiazol-2-yl}phenyl)-1,2,3,6-tetrahydropyridine (100 mg, 0.13 mmol), N—((N′,N″-bis-tert-butyloxycarbonyl)amidino)pyrrazole (121 mg, 0.39 mmol) and N,N-diisopropyl ethylamine (168 mg, 1.3 mmol, 233 μl) in methanol (2 mL) was stirred for 18 hours. The product was purified by reverse phase HPLC. The product fractions were combined, treated with saturated sodium bicarbonate solution, extracted with dichloromethane (2×50 mL), dried and evaporated to leave product as a glassy solid. The product was dissolved in 1,4-dioxane (2 mL) and 4N HCl/1,4-dioxane (2 mL). After stirring for 18 hours the solvents were evaporated and the product was lyophilized to leave a white solid (57 mg, 25%). ¹H-NMR (CD₃OD) δ 8.16 (d, 2H, J=8.8 Hz), 8.02 (s, 1H), 7.97 (d, 1H, J=8.2 Hz), 7.72 (d, 2H, J=8.8 Hz), 7.37 (d, 1H, J=8.2 Hz), 6.37 (m, 1H), 5.76 (m, 1H), 4.18 (m, 4H), 3.75 (m, 4H), 2.75 (m, 2H), 2.58 (m, 2H), 2.44 (s, 3H). LCMS method A R_{f}=3.08 mins, purity >95%, (M+H)⁺=483.

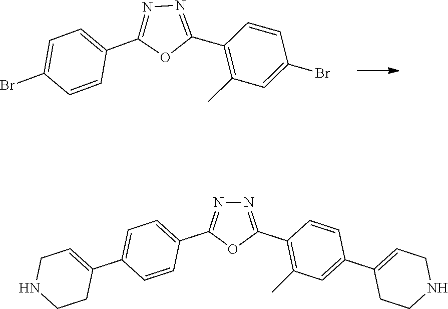 [SMILES: Cc1cc(Br)ccc1-c1nnc(-c2ccc(Br)cc2)o1 ; Cc1cc(C2=CCNCC2)ccc1-c1nnc(-c2ccc(C3=CCNCC3)cc2)o1]

4-Bromo-N′-(4-bromobenzoyl)-2-methylbenzohydrazide was prepared from 4-bromo-2-methylbenzoyl chloride and 4-bromo-benzoic acid hydrazide.

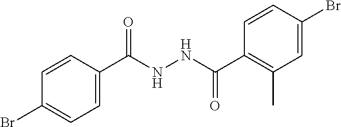 [SMILES: Cc1cc(Br)ccc1C(=O)NNC(=O)c1ccc(Br)cc1]

2-(4-Bromo-2-methylphenyl)-5-(4-bromophenyl)-1,3,4-oxadiazole was prepared from 4-bromo-N′-(4-bromobenzoyl)-2-methylbenzohydrazide and phosphorus oxychloride.

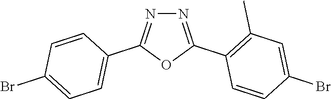 [SMILES: Cc1cc(Br)ccc1-c1nnc(-c2ccc(Br)cc2)o1]

Synthesis of 4-(3-Methyl-4-{5-[4-(1,2,3,6-tetrahydropyridin-4-yl)phenyl]-1,3,4-oxadiazol-2-yl}phenyl)-1,2,3,6-tetrahydropyridine: A mixture of 2-(4-bromo-2-methyl phenyl)-5-(4-bromophenyl)-1,3,4-oxadiazole (100 mg, 0.25 mmol), tert-butyl 4-(4,4,5,5-tetramethyl-1,3,2-dioxaborolan-2-yl)-1,2,3,6-tetrahydropyridine-1-carboxylate (232 mg, 0.75 mmol), dicyclohexyl(2′,6′-dimethoxybiphenyl-2-yl) phosphine (16 mg, 18 μmol), palladium acetate (4.0 mg, 18 μmol), potassium carbonate (260 mg, 1.9 mmol), water (0.8 mL) and 1,4-dioxane (2 mL) was purged with nitrogen, stirred and heated to 90° C. for 20 hours. The mixture was diluted with dichloromethane (20 mL) and water (20 mL), separated and the aqueous layer was extracted with dichloromethane (20 mL). The combined dichloromethane layers were dried (Na₂SO₄) and evaporated. The crude product mixture was dissolved in trifluoroacetic acid (1 mL) and dichloromethane (1 mL) and stirred for 2 h. The solvents were evaporated and the crude product was purified by preparative reverse phase HPLC and the product fractions were combined and lyophilized to leave a white solid (111 mg, 60%). ¹H-NMR (CD₃OD) δ 8.17 (d, 2H, 3=8.5 Hz), 8.08 (d, 1H, J=8.2 Hz), 7.74 (d, 2H, J=8.5 Hz), 7.57 (s, 1H), 7.55 (d, 1H, J=8.2 Hz), 6.35 (m, 2H), 3.90 (m, 4H), 3.52 (m, 4H), 2.86 (m, 4H), 2.78 (s, 3H). LCMS method A R_{f}=2.69 mins, purity >95%, (M+H)⁺=399.

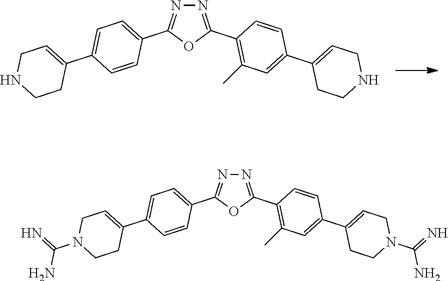 [SMILES: Cc1cc(C2=CCNCC2)ccc1-c1nnc(-c2ccc(C3=CCNCC3)cc2)o1 ; Cc1cc(C2=CCN(C(=N)N)CC2)ccc1-c1nnc(-c2ccc(C3=CCN(C(=N)N)CC3)cc2)o1]

### Example 43

4-(4-(5-(4-(1-Carbamimidoyl-1,2,3,6-tetrahydropyridin-4-yl)-2-methylphenyl)-1,3,4-oxadiazol-2-yl)phenyl)-5,6-dihydropyridine-1(2H)-carboximidamide A solution of 4-(3-methyl-4-{5-[4-(1,2,3,6-tetrahydropyridin-4-yl)phenyl]-1,3,4-oxadiazol-2-yl}phenyl)-1,2,3,6-tetrahydropyridine (105 mg, 0.14 mmol), N″-bis-tert-butyloxycarbonyl)amidino) pyrrazole (130 mg, 0.42 mmol) and N,N-diisopropylethylamine (181 mg, 1.4 mmol, 251 μl) in methanol (2 mL) was stirred for 18 hours. The product was purified by reverse phase HPLC. The product fractions were combined, treated with saturated sodium bicarbonate solution, extracted with methylene chloride (2×50 mL), dried and evaporated to leave product as a gum. The product was dissolved in 1,4-dioxane (1 mL) and 4N HCl/1,4-dioxane (1 mL). After stirring for 18 hours the solvents were evaporated and the product was lyophilized to leave a white solid (78 mg, 31%). ¹H-NMR (CD₃OD) δ 8.13 (d, 2H, J=8.5 Hz), 8.07 (d, 1H, J=8.2 Hz), 7.71 (d, 2H, J=8.5 Hz), 7.54 (s, 1H), 7.52 (d, 1H, J=8.2 Hz), 6.34 (m, 2H), 4.18 (m, 4H), 3.72 (m, 4H), 2.77 (s, 3H), 2.76 (m, 4H). LCMS method A R_{f}=3.12 mins, purity >95%, (M+H)⁺=483. ¹H NMR (300 MHz, METHANOL-d₄) δ=8.29-7.94 (m, 3H), 7.85-7.62 (m, 2H), 7.51 (br d, J=1.8 Hz, 2H), 6.63-6.22 (m, 2H), 4.18 (t, J=3.1 Hz, 4H), 3.90-3.65 (m, 4H), 2.79 (br s, 3H). LC/MS method A: R_{f}=3.12 mins., (M+H)⁺=483, purity >95%.

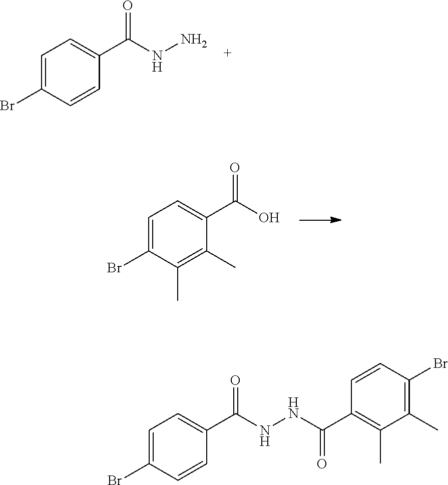 [SMILES: NNC(=O)c1ccc(Br)cc1 ; Cc1c(Br)ccc(C(=O)O)c1C ; Cc1c(Br)ccc(C(=O)NNC(=O)c2ccc(Br)cc2)c1C]

Synthesis of 4-bromo-N′-(4-Bromobenzoyl)-2,3-dimethylbenzohydrazide: A solution of 4-bromophenylhydrazide (0.50 g, 2.3 mmol), 4-bromo-2,3-dimethylbenzoic acid (0.53 g, 2.3 mmol), 1-hydroxy-7-azabenzotriazole (0.31 g, 2.3 mmol) and N, N-diisopropylethylamine (0.30 g, 2.3 mmol, 0.41 mL) in N,N-dimethylformamide (5 mL) was treated with 1-Ethyl-3-(3-dimethylaminopropyl)carbodiimide (0.44 g, 2.3 mmol) and stirred for 5 hours. The reaction mixture was diluted with water (10 mL), cooled in an ice bath and stirred for 30 minutes. The precipitate was filtered, washed with water and dried under vacuum to leave 0.84 g (86%) of light gray solid.

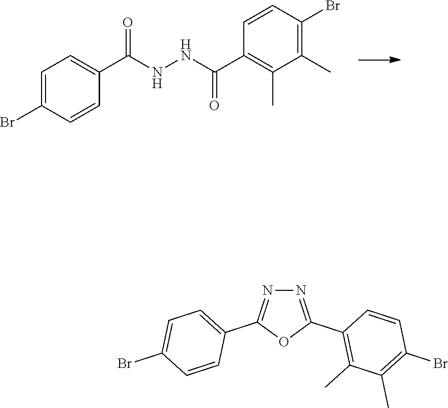 [SMILES: Cc1c(Br)ccc(C(=O)NNC(=O)c2ccc(Br)cc2)c1C ; Cc1c(Br)ccc(-c2nnc(-c3ccc(Br)cc3)o2)c1C]

Synthesis of 2-(4-Bromo-2,3-dimethylphenyl)-5-(4-bromophenyl)-1,3,4-oxadiazole: A solution of 4-bromo-N′-(4-bromobenzoyl)-2,3-dimethylbenzohydrazide (0.43 g, 1.0 mmol) in acetonitrile (15 mL) and phosphorous oxychloride (3 mL) was stirred at 80° C. for 5 hours. The reaction mixture was cooled in an ice bath, treated with ice (5 g) and stirred for 30 minutes. Additional water (10 mL) was added and the mixture was filtered, washed with water and the solid was dried under vacuum to leave 0.42 g (100%). ¹H-NMR (DMSO-d₆) δ 8.03 (d, 2H, J=8.5 Hz), 7.83 (d, 2H, J=8.5 Hz), 7.75 (d, 1H, J==8.8 Hz), 7.68 (d, 1H, J=8.8 Hz), 2.65 (s, 3H), 2.45 (s, 3H). LCMS method A R_{f}=6.84 mins, purity >95%, (M+H)³⁰=409.

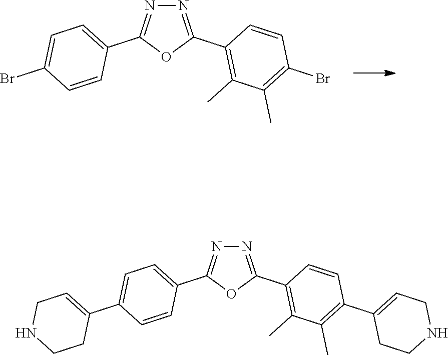 [SMILES: Cc1c(Br)ccc(-c2nnc(-c3ccc(Br)cc3)o2)c1C ; Cc1c(C2=CCNCC2)ccc(-c2nnc(-c3ccc(C4=CCNCC4)cc3)o2)c1C]

Synthesis of 4-(2,3-Dimethyl-4-{5-[4-(1,2,3,6-tetrahydropyridin-4-yl)phenyl]-1,3,4-oxadiazol-2-yl}phenyl)-1,2,3,6-tetrahydropyridine: A mixture of 2-(4-bromo-2,3-dimethylphenyl)-5-(4-bromophenyl)-1,3,4-oxadiazole (102 mg, 0.25 mmol), tert-butyl 4-(4,4,5,5-tetramethyl-1,3,2-dioxaborolan-2-yl)-1,2,3,6-tetrahydropyridine-1-carboxylate (232 mg, 0.75 mmol), dicyclohexyl(2′,6′-dimethoxybiphenyl-2-yl) phosphine (16 mg, 18 μmol), palladium acetate (4.0 mg, 18 μmol), potassium carbonate (260 mg, 1.9 mmol), water (0.8 mL) and 1,4-dioxane (2 mL) was purged with nitrogen, stirred and heated to 90° C. for 20 h. The mixture was diluted with dichloromethane (20 mL) and water (20 mL), separated and the aqueous layer was extracted with dichloromethane (20 mL). The combined dichloromethane layers were dried (Na₂SO₄) and evaporated. The crude product mixture was dissolved in trifluoroacetic acid (1 mL) and dichloromethane (1 mL) and stirred for 2 hours. The solvents were evaporated and the crude product was purified by preparative reverse phase HPLC and the product fractions were combined and lyophilized to leave a white solid (106 mg, 56%). ¹H-NMR. (CD₃OD) δ 8.16 (d, 2H, J=8.8 Hz), 7.81 (d, 1H, J=7.9 Hz), 7.74 (d, 2H, J=8.8 Hz), 7.20 (d, 1H, J=7.9 Hz), 6.36 (m, 1H), 5.71 (m, 1H), 3.90 (m, 4H), 3.52 (m, 4H), 2.88 (m, 2H), 2.65 (s, 3H), 2.61 (m, 2H), 2.38 (s, 3H). LCMS method A R_{f}=2.77 mins, purity >85%, (M+H)⁺=413.

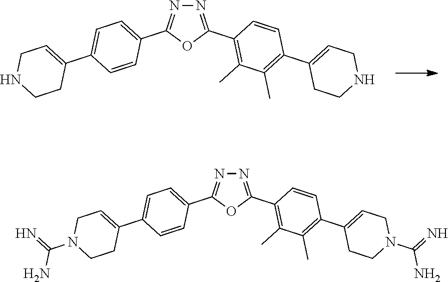 [SMILES: Cc1c(C2=CCNCC2)ccc(-c2nnc(-c3ccc(C4=CCNCC4)cc3)o2)c1C ; Cc1c(C2=CCN(C(=N)N)CC2)ccc(-c2nnc(-c3ccc(C4=CCN(C(=N)N)CC4)cc3)o2)c1C]

### Example 44

4-(4-(5-(4-(1-Carbamimidoyl-1,2,3,6-tetrahydropyridin-4-yl)-2,3-dimethylphenyl)-1,3,4-oxadiazol-2-yl)phenyl)-5,6-dihydropyridine-1(2H)-carboximidamide: A solution of 4-(2,3-dimethyl-4-{5-[4-(1,2,3,6-tetrahydropyridin-4-yl) phenyl]-1,3,4-oxadiazol-2-yl} phenyl)-1,2,3,6-tetrahydropyridine (100 mg, 0.13 mmol), N—((N′,N″-bis-tert-butyloxycarbonyl) amidino)pyrrazole (121 mg, 0.39 mmol) and N,N-(diisopropyl)ethylamine (168 mg, 1.3 mmol, 233 μl) in methanol (2 mL) was stirred for 18 hours. The product mixture was purified by reverse phase HPLC. The product fractions were combined, treated with saturated sodium bicarbonate solution, extracted with methylene chloride (2×50 mL), dried and evaporated to leave the product as a gum. The product was dissolved in 1,4-dioxane (2 mL) and 4N HCl/1,4-dioxane (2 mL). After stirring for 18 hours the solvents were evaporated and the product was lyophilized to leave a white solid (78 mg, 33%). ¹H-NMR (CD₃OD) δ 8.13 (d, 2H, J=8.5 Hz), 7.80 (d, 1H, J=7.9 Hz), 7.71 (d, 2H, J=8.5 Hz), 7.19 (d, 1H, J=7.9 Hz), 6.38 (m, 1H), 5.71 (m, 1H), 4.18 (m, 4H), 3.73 (m, 4H), 2.75 (m, 2H), 2.65 (s, 3H), 2.54 (m, 2H), 2.34 (s, 3H). LCMS method A R_{f}=3.17 mins, purity >90%, (M+H)⁺=497.

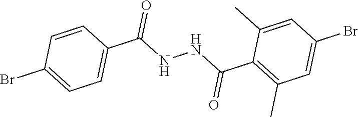 [SMILES: Cc1cc(Br)cc(C)c1C(=O)NNC(=O)c1ccc(Br)cc1]

Synthesis of 4-Bromo-N′-(4-bromobenzoyl)-2,6-dimethylbenzoh-ydrazide: A flask was charged with 856 mg (4.00 mmol) of 4-bromobenzohydrazide and slurried with 4 mL of N,N-dimethylformamide and 808 mg (8.00 mmol) of triethylamine and cooled in an ice water bath. Then a solution of 4-bromo-2,6-dimethylbenzoyl chloride (1040 mg, 4.21 mmol) in 3 mL of dichloromethane was added dropwise over five minutes. The reaction was allowed to stir for 18 hours at 23° C. The reaction was charged with 35 mL of water and stirred for one hour. Solid was collected on funnel and rinsed with five mL of methanol. After drying yield was 1380 mg (81%).

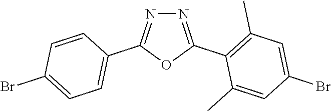 [SMILES: Cc1cc(Br)cc(C)c1-c1nnc(-c2ccc(Br)cc2)o1]

Synthesis of 2-(4-Bromo-2,6-dimethylphenyl)-5-(4-bromophenyl)-1,3,4-oxadiazole: A flask was charged with 1380 mg (3.25 mmol) of 4-bromo-N-(4-bromo benzoyl)-2-fluorobenzohydrazide hydrate. The solid was slurried with 10 mL toluene and brief sonicated. Then 1680 mg (7.81 mmol) of PCl₅ was added at once. The reaction was heated to 99° C. for 18 hours. The reaction was cooled and concentrated under vacuum to yield a solid. The reaction was quenched by addition of wet ice (about 10 grams). After a two-hour age water removed by decantation, solid triturated with 5 mL of water and rinsed with 5 mL ether and airdried for 18 hours Yield 1200 mg of solid (80%).

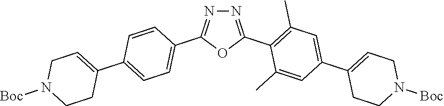 [SMILES: Cc1cc(C2=CCN(C(=O)OC(C)(C)C)CC2)cc(C)c1-c1nnc(-c2ccc(C3=CCN(C(=O)OC(C)(C)C)CC3)cc2)o1]

Synthesis of tert-Butyl 4-(4-(5-(4-(1-(tert-butoxycarbonyl)-1,2,3,6-tetrahydro pyridin-4-yl)-2,6-di methyl phenyl)-1,3,4-oxadiazol-2-yl)phenyl)-5,6-dihydropyridine-1(2H)-carboxylate: A flask was charged with 350 mg (0.864 mmol) of 2-(4-bromo-2,6-dimethyl phenyl)-5-(4-bromophenyl)-1,3,4-oxadiazole, 16 mg (0.07 mmol) of palladium acetate, 71 mg (0.17 mmol) of S-Phos, 641 mg (2.07 mmol) of tert-butyl 4-(4,4,5,5-tetramethyl-1,3,2-dioxaborolan-2-yl)-5,6-dihydropyridine-1(2H)-carboxylate. 6 mL of dioxane and 2.4 mL of 2.0 molar potassium carbonate aqueous solution. The flask was swept with nitrogen for five minutes and heated at 105° C. for 18 hours. The reaction was complete by LCMS and the reaction was allowed to cool and diluted with 20 mL water and 30 mL of ethyl acetate added over two minutes. Organic phase dried and concentrated under vacuum to yield 400 mg (76%).

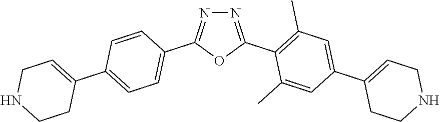 [SMILES: Cc1cc(C2=CCNCC2)cc(C)c1-c1nnc(-c2ccc(C3=CCNCC3)cc2)o1]

Synthesis of 2-(2,6-Dimethyl-4-(1,2,3,6-tetrahydropyridin-4-yl)phenyl)-5-(4-(1,2,3,6-tetrahydropyridin-4-yl)phenyl)-1,3,4-oxadiazole: A flask was charged with 400 mg (0.653 mmol) of the tert-butyl 4-(4-(5-(4-(1-(tert-butoxycarbonyl)-1,2,3,6-tetrahydropyridin-4-yl)-2,6-dimethylphenyl)-1,3,4-oxadiazol-2-yl)phenyl)-5,6-dihydropyridine-1(2H)-carboxylate which was dissolved with 5 mL dichloromethane. Then 6 mL (78 mmol) of trifluoroacetic acid added over five minutes with stirring. Two hours later the reaction was concentrated under vacuum and triturated with two 20 mL portions of ether and ether decanted away to yield a solid in quantitative yield.

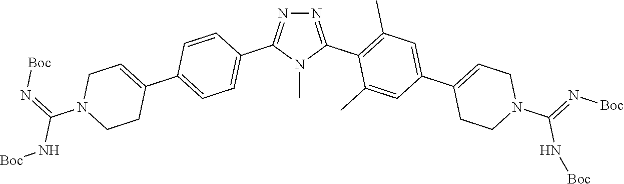 [SMILES: Cc1cc(C2=CCN(/C(=N/C(=O)OC(C)(C)C)NC(=O)OC(C)(C)C)CC2)cc(C)c1-c1nnc(-c2ccc(C3=CCN(/C(=N\C(=O)OC(C)(C)C)NC(=O)OC(C)(C)C)CC3)cc2)n1C]

Synthesis of tert-Butyl N-[(E)-(4-{4-[5-(4-{1-[(Z)-{[(tert-butoxy)carbonyl]amino}-({[(tert-butoxy)carbonyl]imino})methyl]-1,2,3,6-tetrahydropyridin-4-yl}-2,6-dimethylphenyl)-1,3,4-oxadiazol-2-yl]phenyl}-1,2,3,6-tetrahydropyridin-1-yl)({[(tert-butoxy)carbonyl]imino}) methyl]carbamate: A flask was charged with 384 mg (0.60 mmol) of 2-(2,6-dimethyl-4-(1,2,3,6-tetrahydropyridin-4-yl)phenyl)-5-(4-(1,2,3,6-tetrahydropyridin-4-yl)phenyl)-1,3,4-oxadiazole bis trifluoroacetic acid salt and 6 mL, dichloromethane Then 560 mg (5.54 mmol) of triethylamine added over two minutes. Then 484 mg (1.56 mmol) of tert-butyl (1H-pyrazol-1-yl)methanediylidenedicarbamate was added. The reaction was stirred at room temp for 18 hours. The reaction was diluted with 40 mL ethyl acetate and then washed with two portions of 20 mL of water. Organic phase was dried with sodium sulfate and concentrated under vacuum to yield 570 mg of product in quantitative yield.

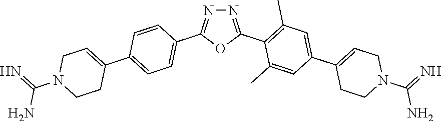 [SMILES: Cc1cc(C2=CCN(C(=N)N)CC2)cc(C)c1-c1nnc(-c2ccc(C3=CCN(C(=N)N)CC3)cc2)o1]

### Example 45

Synthesis of 4-(4-(5-(4-(1-Carbamimidoyl-1,2,3,6-tetrahydropyridin-4-yl)-2,6-dimethyl phenyl)-1,3,4-oxadiazol-2-yl)phenyl)-5,6-dihydropyridine-1(2H)-carboximidamide: A flask was charged with 570 mg (0.600 mmol) of tert-butyl N-[(E)-(4-{4-[5-(4-{1-[(Z)-{[(tert-butoxy)carbonyl]amino}-({[(tert-butoxy)carbonyl]imino})methyl]-1,2,3,6-tetrahydropyridin-4-yl}-2,6-dimethylphenyl)-1,3,4-oxadiazol-2-yl]phenyl}-1,2,3,6-tetrahydropyridin-1-yl)({[(tert-butoxy) carbonyl]imino})methyl]carbamate which was dissolved with 4 mL of dichloromethane. Then 4 mL (52 mmol) of trifluoroacetic acid added over two minutes with stirring. After two hours the LCMS indicted reaction complete and reaction concentrated under vacuum. The residual oil was triturated with 20 mL portions of ether and 20 mL ethyl acetate. Solid was dissolved in N,N-dimethylformamide and was purified by prep HPLC. Yield was 44 mg (10%). ¹H NMR (300 MHz, DMSO-d₆) δ=7.85-7.63 (m, 2H), 7.62-7.31 (m, 4H), 6.27 (br t, J=2.5 Hz, 2H), 4.16-4.00 (m, 2H), 3.86-3.51 (m, 2H), 3.30-3.01 (m, 2H), 2.92-2.67 (m, 2H), 2.66-2.54 (m, 2H), 2.41 (s, 6H). LC/MS method A: R_{f}=3.17 mins., (M+H)⁺=497, purity >95%.

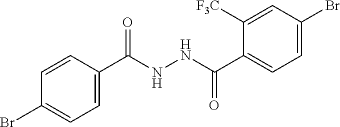 [SMILES: O=C(NNC(=O)c1ccc(Br)cc1C(F)(F)F)c1ccc(Br)cc1]

Synthesis of 4-Bromo-N′-(4-bromobenzoyl)-2-(trifluoromethyl)benzohydrazide: A flask was charged with 1500 mg (7.00 mmol) of 4-bromobenzohydrazide and dissolved with 25 mL of N,N-dimethylformamide and 1061 mg (10.50 mmol) of triethylamine and cooled in an ice water bath. Then a solution of 4-bromo-2-(trifluoromethyl)benzoyl chloride (2100 mg, 7.35 mmol) was added dropwise over 15 minutes. The reaction was allowed to stir for 18 hours at 23° C. The reaction was charged with 70 mL of water and stirred for 1.5 hours. Solid was collected on filter funnel and rinsed with 15 mL of water followed by 5 mL, of ether. After drying yield was 1300 mg (40%).

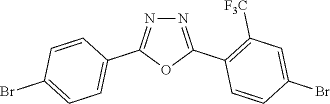 [SMILES: FC(F)(F)c1cc(Br)ccc1-c1nnc(-c2ccc(Br)cc2)o1]

Synthesis of 2-(4-Bromo-2-(trifluoromethyl)phenyl)-5-(4-bromophenyl)-1,3,4-oxadiazole: A flask was charged with 1300 mg (2.79 mmol) of 4-bromo-N′-(4-bromobenzoyl)-2-(trifluoromethyl)benzohydrazide hydrate. The solid was slurried with 10 mL toluene and briefly sonicated. Then 1400 mg (6.70 mmol) of PCl₅ was added at once. The reaction was heated to 95° C. for 18 hours. The reaction is a solution after heating for 18 hours. The reaction was cooled and concentrated under vacuum to yield a solid. The reaction was quenched by addition of wet ice (about 15 grams). After a two-hour age water removed by decantation, solid triturated with 5 mL of water and rinsed with 10 mL ether. Ether phase concentrated under vacuum and 30 mL ethyl acetate added and removed under vacuum to yield 710 mg of solid (55%).

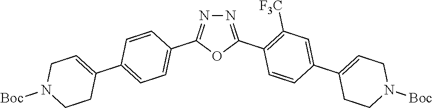 [SMILES: CC(C)(C)OC(=O)N1CC=C(c2ccc(-c3nnc(-c4ccc(C5=CCN(C(=O)OC(C)(C)C)CC5)cc4C(F)(F)F)o3)cc2)CC1]

Synthesis of tert-Butyl 4-(4-(5-(4-(1-(tert-butoxycarbonyl)-1,2,3,6-tetrahydro pyridin-4-yl)-2-(trifluoromethyl)phenyl)-1,3,4-oxadiazol-2-yl)phenyl)-5,6-dihydropyridine-1(2H)-carboxylate: A flask was charged with 400 mg (0.896 mmol) of 2-(4-bromo-2-(trifluoro methyl)phenyl)-5-(4-bromophenyl)-1,3,4-oxadiazole, 16 mg (0.07 mmol) of palladium acetate, 74 mg (0.18 mmol) of 2-dicyclohexylphosphino-2′,6′-dimethoxybiphenyl, 651 mg (2.10 mmol) of tert-butyl 4-(4,4,5,5-tetramethyl-1,3,2-dioxaborolan-2-yl)-5,6-dihydro pyridine-1(2H)-carboxylate, 7 mL of dioxane and 3.4 mL of 2.0 molar potassium carbonate aqueous solution. The flask was swept with nitrogen for five minutes and heated at 95° C. for 18 hours. The reaction was complete by LCMS and the reaction was allowed to cool and diluted with 10 mL water and 20 mL of ethyl acetate added over two minutes. Organic phase dried and concentrated under vacuum and concentrate dissolved with dichloromethane and chromatographed eluting with (20% isopropanol/dichloromethane)/dichloromethane to yield 400 mg (68%).

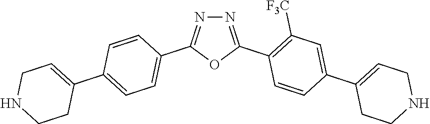 [SMILES: FC(F)(F)c1cc(C2=CCNCC2)ccc1-c1nnc(-c2ccc(C3=CCNCC3)cc2)o1]

Synthesis of 2-(4-(1,2,3,6-Tetrahydropyridin-4-yl)-2-(trifluoromethyl)phenyl)-5-(4-(1,2,3,6-tetrahydropyridin-4-yl)phenyl)-1,3,4-oxadiazole: A flask was charged with 400 mg (0.6.13 mmol) of the tert-butyl 4-(4-(5-(4-(1-(tert-butoxycarbonyl)-1,2,3,6-tetrahydro pyridin-4-yl)-2-(trifluoromethyl)phenyl)-1,3,4-oxadiazol-2-yl)phenyl)-5,6-dihydropyridine-1(2H)-carboxylate which was dissolved with mL of dichloromethane. Then 6 mL (78 mmol) of trifluoroacetic acid added over five minutes with stirring. Three hours later the reaction was concentrated under vacuum and triturated with two 20 mL portions of ether and ether decanted away to yield a solid in quantitative yield.

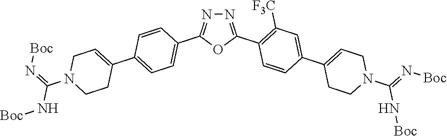 [SMILES: CC(C)(C)OC(=O)/N=C(/NC(=O)OC(C)(C)C)N1CC=C(c2ccc(-c3nnc(-c4ccc(C5=CCN(/C(=N/C(=O)OC(C)(C)C)NC(=O)OC(C)(C)C)CC5)cc4C(F)(F)F)o3)cc2)CC1]

Synthesis of tert-Butyl N-[(E)-(4-{4-[5-(4-{1-[(Z)-{[-(tert-butoxy)carbonyl] amino}({[(tert-butoxy)carbonyl]imino})methyl]-1,2,3,6-tetrahydropyridin-4-yl}-2-(trifluoromethyl) phenyl)-1,3,4-oxadiazol-2-yl]phenyl}-1,2,3,6-tetrahydropyridin-1-yl)({[(tert-butoxy)carbonyl] imino})methyl]carbamate: A flask was charged with 340 mg (0.50 mmol) of 2-(4-(1,2,3,6-tetrahydropyridin-4-yl)-2-(trifluoromethyl)phenyl)-5-(4-(1,2,3,6-tetrahydropyridin-4-yl) phenyl)-1,3,4-oxadiazole bis trifluoroacetic acid salt, 2 mL N,N-dimethylformamide and 5 mL dichloromethane. Then 490 mg (4.85 mmol) of triethylamine added over two minutes. Then 430 mg (1.39 mmol) of tert-butyl. (1H-pyrazol-1-yl)methanediylidenedicarbamate was added. The reaction was stirred for 24 hours at 23° C. Then 130 mg (0.419 mmol) of tert-butyl (1H-pyrazol-1-yl)methanediylidenedicarbamate and 140 mg (1.39 mmol) of triethylamine was added. After 24 hours the reaction was diluted with 40 mL ethyl acetate and then washed with 20 mL of water. Organic phase was dried with sodium sulfate and concentrated under vacuum. The precut was purified by normal phase chromatography, loaded as dichloromethane solution, eluted with (15% isopropanol/dichloromethane)/dichloromethane with step gradient from 0 to 50% to elute product. Yield was 313 mg of product (67%).

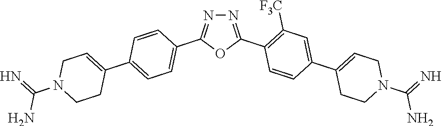 [SMILES: N=C(N)N1CC=C(c2ccc(-c3nnc(-c4ccc(C5=CCN(C(=N)N)CC5)cc4C(F)(F)F)o3)cc2)CC1]

### Example 46

Synthesis of 4-(4-(5-(4-(1-Carbamimidoyl-1,2,3,6-tetrahydropyridin-4-yl)-2-(trifluoromethyl)phenyl)-1,3,4-oxadiazol-2-yl)phenyl)-5,6-dihydropyridine-1(2H)-carboximidamide: A flask was charged with 313 mg (0.335 mmol) of tert-butyl N-[(E)-(4-{4-[5-(4,1-[(Z)-{[-(tert-butoxy)carbonyl]amino)({([(tert-butoxy)carbonyl]imino})methyl]-1,2,3,6-tetrahydro pyridin-4-yl}-2-(trifluoromethyl)phenyl)-1,3,4-oxadiazol-2-yl]phenyl}-1,2,3,6-tetrahydropyridin-1-yl)({[(tert-butoxy)carbonyl]imino})methyl]carbamate which was dissolved with 3 mL of dichloromethane. Then 5 mL (77.9 mmol) of trifluoro acetic acid added over two minutes with stirring and stirred for 24 hours. The LCMS indicted reaction was complete and reaction concentrated under vacuum. Concentrate was dissolved in 80% N,N-dimethylformamide/water and was purified by prep HPLC. Yield was 34 mg (13%) ¹H NMR (300 MHz, DMSO-d₆) δ=8.20 (d, J=8.1 Hz, 1H), 8.06-7.98 (m, 2H), 7.75 (d, J=7.6 Hz, 1H), 7.53-7.38 (m, 3H), 6.58 (br t, J=2.6 Hz, 1H), 6.40 (br s, 1H), 4.19-4.07 (m, 2H), 3.71-3.54 (m, 2H), 3.51-3.39 (m, 2H), 3.27-3.06 (m, 2H), 2.73-2.60 (m, 2H). LC/MS method A: R_{f}=3.50 mins., (M+H)⁺=379, purity >95%.

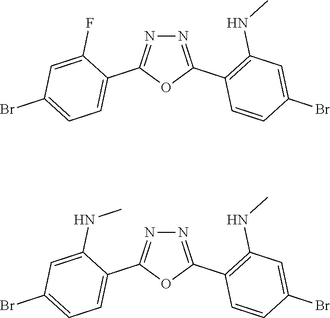 [SMILES: CNc1cc(Br)ccc1-c1nnc(-c2ccc(Br)cc2F)o1 ; CNc1cc(Br)ccc1-c1nnc(-c2ccc(Br)cc2NC)o1]

Synthesis of 6,6′-(1,3,4-Oxadiazole-2,5-diyl)bis(3-bromo-N-methylaniline) and 5-Bromo-2-(5-(4-bromo-2-fluorophenyl)-1,3,4-oxadiazol-2-yl)-N-methylaniline: A pressure flask was charged with 5-Bromo-2-(5-(4-bromo-2-fluorophenyl)-1,3,4-oxadiazole (1300 mg, 3.25 mmol) was slurried with 5 mL of 1,4-dioxane and the methylamine (388 mg, 12.5 mol), 33% in ethanol was added over two minutes. The reaction was heated to 90° C. for 18 hours. The reaction was allowed to cool and most of methanol removed under vacuum. Then 10 mL of water and 4 mL of ethyl acetate were added and sonicated for ten minutes. One hour later the solid was collected and discarded. The biphasic mother liquor was diluted with 80 mL ethyl acetate, dried and concentrated to yield was 1.10 grams of a mixture of products (88%).

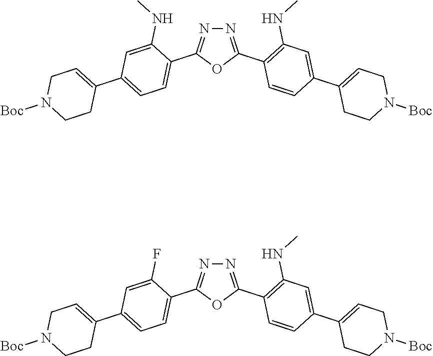 [SMILES: CNc1cc(C2=CCN(C(=O)OC(C)(C)C)CC2)ccc1-c1nnc(-c2ccc(C3=CCN(C(=O)OC(C)(C)C)CC3)cc2NC)o1 ; CNc1cc(C2=CCN(C(=O)OC(C)(C)C)CC2)ccc1-c1nnc(-c2ccc(C3=CCN(C(=O)OC(C)(C)C)CC3)cc2F)o1]

Synthesis of tert-Butyl 4,4′-(4,4′-(1,3,4-oxadiazole-2,5-diyl)bis(3-(methylamino)-4,1-phenylene))bis(5,6-dihydropyridine-1(2H)-carboxylate 6,6′-(1,3,4-oxadiazole-2,5-diyl)bis(3-bromo-N-methylaniline) and tert-Butyl 4-(4-(5-(4-(1-(tert-butoxycarbonyl)-1,2,3,6-tetrahydropyridin-4-yl)-2-(methylamino)phenyl)-1,3,4-oxadiazol-2-yl)-3-fluorophenyl)-5,6-dihydropyridine-1(2H)-carboxylate: A flask was charged with 1100 mg (2.58 mmol) of a mixture of 5-bromo-2-(5-(4-bromo-2-fluorophenyl)-1,3,4-oxadiazol-2-yl)-N-methylaniline and 6,6′-(1,3,4-oxadiazole-2,5-diyl)bis(3-bromo-N-methylaniline) and tert-butyl 4-(4,4,5,5-tetramethyl-1,3,2-dioxaborolan-2-yl)-5,6-dihydropyridine-1(2H)-carboxylate, 46 mg (0.206 mmol) of palladium acetate, 212 mg (0.516 mmol) of 2-dicyclohexylphosphino-2′,6′-dimethoxybiphenyl, 1830 mg (5.92 mmol) of tert-butyl 4-(4,4,5,5-tetramethyl-1,3,2-dioxaborolan-2-yl)-5,6-dihydropyridine-1(2H)-carboxylate, 22 mL of 1,4-dioxane and 10 mL of 2.0 molar potassium carbonate aqueous solution. The flask was swept with nitrogen for five minutes and heated at 90° C. for 18 hours. The reaction was allowed to cool and charged with, 38 mg (169 mmol) of palladium acetate, 2.10 mg (0.512 mmol) of 2-dicyclohexylphosphino-2′,6′-dimethoxybiphenyl, 850 mg (2.75 mmol) of tert-butyl 4 (4,4,5,5-tetramethyl-1,3,2-dioxaborolan-2-yl)-5,6-dihydropyridine-1(2H)-carboxylate, and 4 mL of 2.0 molar potassium carbonate aqueous solution. The flask was swept with nitrogen for five minutes and heated at 95° C. for 18 hours. Then the reaction was complete by LCMS and the reaction was allowed to cool and was diluted with 30 mL water and 15 mL, of ethyl acetate added over two minutes. Solids formed quickly and reaction stirred for two hours. Solid collected on a filter and air-dried in the hood to yield 350 mg (25%) of two products. The ethyl acetate mother liquor was dried and concentrated to yield an additional 890 mg of a mixture of products (53%).

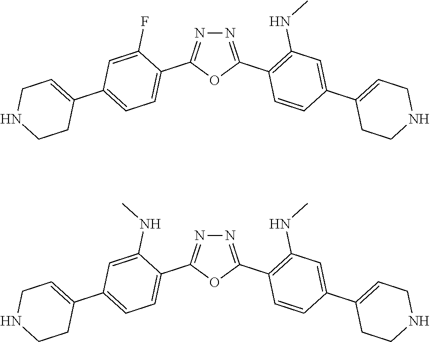 [SMILES: CNc1cc(C2=CCNCC2)ccc1-c1nnc(-c2ccc(C3=CCNCC3)cc2F)o1 ; CNc1cc(C2=CCNCC2)ccc1-c1nnc(-c2ccc(C3=CCNCC3)cc2NC)o1]

Synthesis of 2-(5-(2-Fluoro-4-(1,2,3,6-tetrahydropyridin-4-yl)phenyl)-1,3,4-oxadiazol-2-yl)-N-methyl-5-(1,2,3,6-tetrahydropyridin-4-yl)aniline and 6,6′-(1,3,4-Oxadiazole-2,5-diyl)bis(N-methyl-3-(1,2,3,6-tetrahydropyridin-4-yl)aniline: A flask was charged with 890 mg (1.37 mmol) of a mixture of the tert-butyl 4-(4-(5-(4-(1-(tert-butoxycarbonyl)-1,2,3,6-tetrahydropyridin-4-yl)-2-(methylamino)phenyl)-1,3,4-oxadiazol-2-yl)-3-fluorophenyl)-5,6-dihydropyridine-1(2H)-carboxylate and tert-butyl 4,4′-(4,4′-(1,3,4-oxadiazole-2,5-diyl)bis(3-(methylamino)-4,1-phenylene))bis(5,6-dihydro pyridine-1 (2H)-carboxylate-6,6′-(1,3,4-oxadiazole-2,5-diyl)bis(3-bromo-N-methylaniline) which was slurried with 4 mL dichloromethane. Then 6 mL (78 mmol) of trifluoroacetic acid added over five minutes with stirring. Three hours later the reaction was concentrated under vacuum and triturated with two 30 mL portions of ether and ether decanted away to yield a solid in quantitative yield of a mixture of two products.

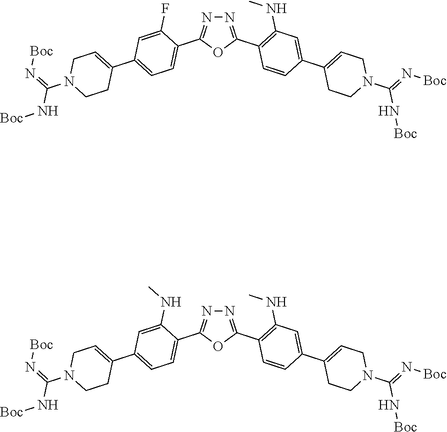 [SMILES: CNc1cc(C2=CCN(/C(=N/C(=O)OC(C)(C)C)NC(=O)OC(C)(C)C)CC2)ccc1-c1nnc(-c2ccc(C3=CCN(/C(=N\C(=O)OC(C)(C)C)NC(=O)OC(C)(C)C)CC3)cc2F)o1 ; CNc1cc(C2=CCN(/C(=N\C(=O)OC(C)(C)C)NC(=O)OC(C)(C)C)CC2)ccc1-c1nnc(-c2ccc(C3=CCN(/C(=N/C(=O)OC(C)(C)C)NC(=O)OC(C)(C)C)CC3)cc2NC)o1]

Synthesis of tert-Butyl N—[(Z)-(4-{4-[5-(4-{1-[(Z)-{[(tert-butoxy)carbonyl]amino}({[(tert-butoxy)carbonyl]imino})methyl]-1,2,3,6-tetrahydropyridin-4-yl}-2-fluorophenyl)-1,3,4-oxadiazol-2-yl]-3-(methylamino)phenyl}-1,2,3,6-tetrahydropyridin-1-yl)({[(tert-butoxy)carbonyl] imino})methyl]carbamate and tert-Butyl (4,4′-(4,4′-(1,3,4-oxadiazole-2,5-diyl)bis(3-(methyl amino)-4,1-phenylene))bis(5,6-dihydropyridine-4,1(2H)-diyl))bis(methanetriyl) tetracarbamate A flask was charged with 890 mg (1.37 mmol) of a mixture of 2-(5-(2-fluoro-4-(1,2,3,6-tetrahydropyridin-4-yl)phenyl)-1,3,4-oxadiazol-2-yl)-N-methyl-5-(1,2,3,6-tetrahydropyridin-4-yl)aniline tris trifluoroacetic acid salt and 6,6′-(1,3,4-oxadiazole-2,5-diyl)bis(N-methyl-3-(1,2,3,6-tetrahydropyridin-4-yl)aniline tris trifluoroacetic acid salt, 7 mL N,N-dimethylformamide and 5 mL dichloromethane. Then 1200 mg (11.9 mmol) of triethylamine added over five minutes. Then 1500 mg (4.84 mmol) of tert-butyl (1H-pyrazol-1-yl)methanediylidenedicarbamate was added. The reaction slurry was warmed at 38° C. for 18 hours. Then the reaction was cooled and the dichloromethane was removed under vacuum, 50 mL ethyl acetate and 30 mL water added. The mixture was filtered to remove insoluble material, organic phase washed with 40 mL 10% citric acid, washed with 40 mL water, dried with sodium sulfate and concentrated under vacuum. Concentrate dissolved with 10 ml, dichloromethane, 150 ul of 1-methylpiperazine was added and the reaction was stirred for 18 hours at 23 CC. Then the reaction was concentrated under vacuum, dissolved with 50 mL of ethyl acetate, washed with 40 mL 10% citric acid, 40 mL water, dried and concentrated under vacuum. The material was purified by normal phase chromatography, loaded as dichloromethane solution, eluted with (20% acetonitrile/dichloromethane)/dichloromethane with step gradient from 20 to 100% to elute products. Yield was 390 mg of products (31%).

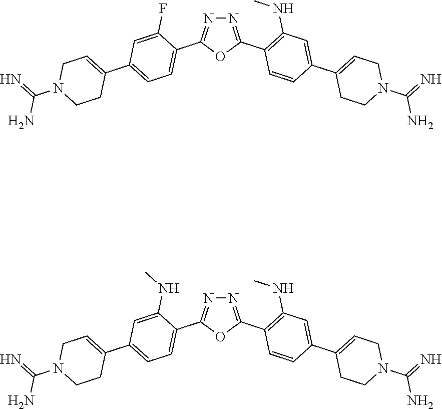 [SMILES: CNc1cc(C2=CCN(C(=N)N)CC2)ccc1-c1nnc(-c2ccc(C3=CCN(C(=N)N)CC3)cc2F)o1 ; CNc1cc(C2=CCN(C(=N)N)CC2)ccc1-c1nnc(-c2ccc(C3=CCN(C(=N)N)CC3)cc2NC)o1]

### Example 47

Synthesis of 4-(4-(5-(4-(1-Carbamimidoyl-1,2,3,6-tetrahydropyridin-4-yl)-2-(methylamino)phenyl)-1,3,4-oxadiazol-2-yl)-3-fluorophenyl)-5,6-dihydropyridine-1(2H)-carboximidamide (47a) and 4,4′-(4,4′-(1,3,4-Oxadiazole-2,5-diyl)bis(3-(methylamino)-4,1-phenylene))bis(5,6-dihydropyridine-1(2H)-carboximidamide) (47b): A flask was charged with 390 mg (0.410 mmol) of a mixture of tert-butyl N—[(Z)-(4-{4-[5-(4-{1-[(Z)-{[(tert-butoxy) carbonyl]amino}({[(tert-butoxy)carbonyl]imino})methyl]-1,2,3,6-tetrahydropyridin-4-yl}-2-fluoro phenyl)-1,3,4-oxadiazol-2-yl]-3-(methylamino)phenyl}-1,2,3,6-tetrahydropyridin-1-yl)({[(tert-butoxy)carbonyl]imino})methyl]carbamate and tert-butyl. (4,4′-(4,4′-(1,3,4-oxadiazole-2,5-diyl)bis(3-(methylamino)-4,1-phenylene))bis(5,6-dihydropyridine-4,1(2H)-diyl))bis(methanetriyl)-tetracarbamate which was dissolved with 4 mL of dichloromethane. Then 6 mL (77.9 mmol) of trifluoroacetic acid added over two minutes with stirring and the reaction was stirred for 18 hours. The LCMS indicated the reaction was complete and reaction concentrated under vacuum. Oil was triturated with two 30 mL portions of ether and ether decanted away to yield a solid. Solid was dissolved in N,N-dimethylformamide and was purified by prep HPLC over 18 minutes. Yield was 76 mg (24%) of 47a and 29 mg (9%) of 47b. 47a NMR (300 MHz, DMSO-d₆) δ=8.19 (t, J=8.0 Hz, 1H), 7.88 (d, J=8.3 Hz, 1H), 7.76-7.54 (m, 1H), 7.54-7.41 (m, 8H), 6.97-6.84 (m, 2H), 6.57 (br t, J=3.1 Hz, 1H), 6.40 (br t, J=3.0 Hz, 1H), 4.20-4.09 (m, 2H), 3.70-3.45 (m, 2H), 3.13-2.98 (m, 2H), 2.67 (br t, J=4.3 Hz, 2H), 2.50-2.42 (m, 2H), 2.37-2.09 (m, 2H). LC/MS method A: R_{f}=3.32 mins., (M+H)⁺=516, purity >95%, 47b ¹H NMR (300 MHz, DMSO-d₆) δ=7.93 (d, J==8.3 Hz, 2H), 7.77-7.56 (m, 2H), 7.48 (br t, J=2.5 Hz, 6H), 7.53-7.39 (m, 2H), 6.94-6.81 (m, 2H), 6.43-6.33 (m, 2H), 4.16-4.05 (m, 2H), 3.68-3.59 (m, 2H), 3.45-3.41 (m, 2H), 3.29-2.93 (m, 6H), 2.74-2.59 (m, 2H), 2.58-2.50 (m, 2H), 2.43-2.15 (m, 2H). LC/MS method A: Rt=3.55 mins., (M+H)+=527, purity >95%.

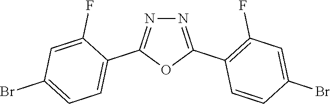 [SMILES: Fc1cc(Br)ccc1-c1nnc(-c2ccc(Br)cc2F)o1]

Synthesis of 2,5-Bis(4-bromo-2-fluorophenyl)-1,3,4-oxadiazole: A flask was charged with 5 mL of 1,2-dichloroethane and 1050 mg (5.05 mmol) of PCl₅ was added. The reaction was stirred at 23° C. for 18 hours. Then 300 mg (0.700 mmol) of (4-bromo-N′-(4-bromo-2-fluorobenzoyl)-2-fluorobenzohydrazide was added and reaction stirred at 23° C. for six hours. The reaction was concentrated under vacuum to yield a solid. The reaction was quenched by addition of 10 grains of wet ice. Five minutes later 5 of ethanol and five mL water added. After 30 minutes, solid collected on a filter. A mixture of 20 mL of acetonitrile and 20 mL ethyl acetate added to solid and removed under vacuum to yield 280 mg of solid (97%).

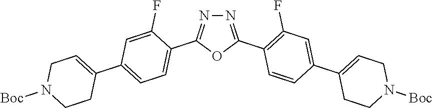 [SMILES: CC(C)(C)OC(=O)N1CC=C(c2ccc(-c3nnc(-c4ccc(C5=CCN(C(=O)OC(C)(C)C)CC5)cc4F)o3)c(F)c2)CC1]

Synthesis of tert-Butyl 4,4′-(4,4′-(1,3,4-oxadiazole-2,5-diyl)bis(3-fluoro-4,1-phenylene)) bis(5,6-dihydropyridine-1(2H)-carboxylate): A flask was charged with 280 mg (0.67 mmol) of 2,5-bis(4-bromo-2-fluorophenyl)-1,3,4-oxadiazole and tert-butyl 4-(4,4,5,5-tetramethyl-1,3,2-dioxaborolan-2-yl)-5,6-dihydropyridine-1(2H)-carboxylate, 12 mg (0.054 mmol) of palladium acetate, 55 mg (0.134 mmol) of 2-dicyclohexylphosphino-2′,6′-dimethoxybiphenyl, 500 mg (1.62 mmol) of tert-butyl 4-(4,4,5,5-tetramethyl-1,3,2-dioxaborolan-2-yl)-5,6-dihydropyridine-1(2H)-carboxylate, 5 of 1,4-dioxane and 2.5 mL, of 2.0 molar potassium carbonate aqueous solution. The flask was swept with nitrogen for five minutes and heated at 95° C. for 18 hours. The reaction was complete by LCMS and the reaction was allowed to cool and diluted with 15 mL water and 5 mL of ethyl acetate added over two minutes. Solids formed quickly and reaction stirred for 30 minutes. Solid collected on a filter and air-dried to yield 360 mg (89%).

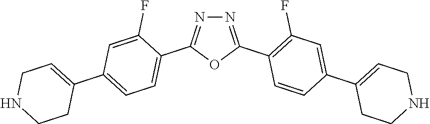 [SMILES: Fc1cc(C2=CCNCC2)ccc1-c1nnc(-c2ccc(C3=CCNCC3)cc2F)o1]

Synthesis of 2,5-Bis(2-fluoro-4-(1,2,3,6-tetrahydropyridin-4-yl)phenyl)-1,3,4-oxadiazole A flask was charged with 360 mg (0.58 mmol) of the tert-butyl 4,4′-(4,4′-(1,3,4-oxadiazole-2,5-diyl)bis(3-fluoro-4,1-phenylene))bis(5,6-dihydropyridine-1(2H)-carboxylate) which was dissolved with 15 mL dichloromethane. Then 4 mL (52 mmol) of trifluoroacetic acid added over two minutes with stirring. Two hours later the reaction was concentrated under vacuum and triturated with two 20 mL portions of ether and ether decanted away to yield a solid in quantitative yield

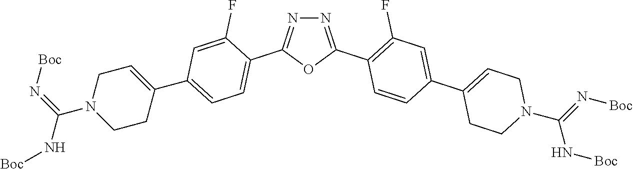 [SMILES: CC(C)(C)OC(=O)/N=C(/NC(=O)OC(C)(C)C)N1CC=C(c2ccc(-c3nnc(-c4ccc(C5=CCN(/C(=N/C(=O)OC(C)(C)C)NC(=O)OC(C)(C)C)CC5)cc4F)o3)c(F)c2)CC1]

Synthesis of tert-Butyl (4,4′-(4,4′-(1,3,4-oxadiazole-2,5-diyl)bis(3-fluoro-4,1-phenylene)) bis(5,6-dihydropyridine-4,1(2H)-diyl))bis(methanetriyl)tetracarbamate: A flask was charged with 375 mg (0.58 mmol) of 2,5-bis(2-fluoro-4-(1,2,3,6-tetrahydropyridin-4-yl)phenyl)-1,3,4-oxadiazole bis trifluoroacetic acid salt, 3 mL dichloromethane and 3 mL N,N-dimethylformamide. Then 352 mg (3.48 mmol) of triethylamine added over two minutes. Then 414 mg (0.1.33 mmol) of tert-butyl (1H-pyrazol-1-yl)methanediylidenedicarbamate was added. The reaction was stirred for 18 hours at 23° C. The reaction was concentrated under vacuum and place on a vacuum pump. Then dissolved with 2 mL dichloromethane and 200 ul of 1-methylpiperazine added. After two hours the reaction was concentrated under vacuum and diluted with 30 mL ethyl acetate and then washed with 20 mL off water, 20 mL of 10% citric and 20 mL water, dried and concentrated under vacuum. The product was purified by normal phase chromatography, loaded as dichloromethane solution, eluted with (20% acetonitrile dichloromethane)/dichloromethane with step gradient from 0 to 100% to elute product. Yield was 140 mg of product (27%).

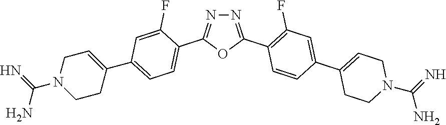 [SMILES: N=C(N)N1CC=C(c2ccc(-c3nnc(-c4ccc(C5=CCN(C(=N)N)CC5)cc4F)o3)c(F)c2)CC1]

### Example 48

Synthesis of 4,4′-(4,4′-(1,3,4-Oxadiazole-2,5-diyl)bis(3-fluoro-4,1-phenylene))bis(5,6-dihydropyridine-1(2H)-carboximidamide: A flask was charged with 140 mg (0.155 mmol) of tert-butyl (4,4′-(4,4′-(1,3,4-oxadiazole-2,5-diyl)bis(3-fluoro-4,1-phenylene))bis(5,6-dihydropyridine-4,1(2H)-diyl))bis(methanetriyl)tetracarbamate which was dissolved with 3 mL of dichloromethane. Then 3 mL (39 mmol) of trifluoroacetic acid added over two minutes with stirring and stirred for 18 hours. LCMS indicated the reaction complete and reaction was concentrated under vacuum. Oil was triturated with two 30 mL portions of ether and ether decanted away to yield a solid. After freeze drying yield was 59 mg (52%). ¹H NMR (300 MHz, DMSO-d₆) δ=8.13 (t. J=8.0 Hz, 2H), 7.71-7.53 (m, 4H), 7.49 (br t, J=2.9 Hz, 6H), 6.56 (br t, J=3.4 Hz, 2H), 4.18-4.07 (m, 4H), 3.69-3.59 (m, 2H), 3.31-3.25 (m, 2H), 2.72-2.54 (m, 2H), 2.48-2.42 (m, 2H). LC/MS method A: Rt=3.04 mins., (M+H)+=505, purity >95%.

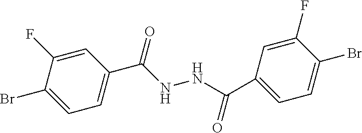 [SMILES: O=C(NNC(=O)c1ccc(Br)c(F)c1)c1ccc(Br)c(F)c1]

Synthesis of 4-Bromo-N′-(4-bromo-3-fluorobenzoyl)-3-fluorobenzohydrazide: A flask was charged with 25 mL of chloroform and cooled in an ice water bath and 388 mg (7.75 mmol) of hydrazine monohydrate, 2400 mg (18.6 mmol) of diisopropylethylamine was added. A solution of 3660 mg (15.5 mmol) of 4-bromo-3-fluorobenzoyl chloride in 20 mL of chloroform was added over one hour and stirred for 18 hours at 23° C. The chloroform was removed under vacuum and the residue was stirred with 70 mL of water for 18 hours after brief sonication. Solids isolated by decantation/filtration. Filter cake/flask rinsed with 30 mL ethyl acetate. Solids from filter added to solids from flask and 50 mL of ethyl acetate added and removed under vacuum to dry solid. Yield was 3200 mg (96%).

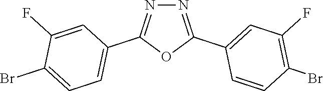 [SMILES: Fc1cc(-c2nnc(-c3ccc(Br)c(F)c3)o2)ccc1Br]

Synthesis of 2,5-Bis(4-bromo-3-fluorophenyl)-1,3,4-oxadiazole: A flask was charged with 15 mL of 1,2-dichloroethane, 570 mg (1.32 mmol) of (4-bromo-N-(4-bromo-3-fluorobenzoyl)-3-fluorobenzohydrazide and 825 mg (3.96 mmol) of PCIs was added. The reaction was stirred at room temperature for five hours. The reaction was concentrated under vacuum to yield a solid. The reaction was quenched by addition of 20 grams of wet ice. After two hours, the water was removed by decantation and 40 mL acetonitrile added and removed under vacuum to yield 360 mg of solid (67%).

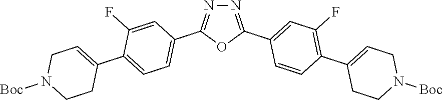 [SMILES: CC(C)(C)OC(=O)N1CC=C(c2ccc(-c3nnc(-c4ccc(C5=CCN(C(=O)OC(C)(C)C)CC5)c(F)c4)o3)cc2F)CC1]

Synthesis of tert-Butyl 4,4′-(4,4′-(1,3,4-oxadiazole-2,5-diyl)bis(2-fluoro-4,1-phenylene)) bis(5,6-dihydropyridine-1(2H)-carboxylate): A flask was charged with 312 mg (0.753 mmol) of 2,5-bis(4-bromo-3-fluorophenyl)-1,3,4-oxadiazole, 14 mg (0.06 mmol) of palladium acetate, 62 mg (0.15 mmol) of 2-dicyclohexylphosphino-2′,6′-dimethoxybiphenyl, 536 mg (1.73 mmol) of tert-butyl 4-(4,4,5,5-tetramethyl-1,3,2-dioxaborolan-2-yl)-5,6-dihydropyridine-1(2H)-carboxylate, 6 mL of dioxane and 2.7 mL of 2.0 molar potassium carbonate aqueous solution. The flask was swept with nitrogen for five minutes and heated at 95° C. for 40 hours. The reaction was allowed to cool and diluted with 20 mL water and 25 mL of ethyl acetate added over two minutes. Organic phase dried and concentrated under vacuum to yield 490 mg in quantitative yield.

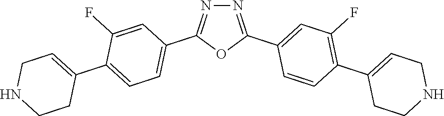 [SMILES: Fc1cc(-c2nnc(-c3ccc(C4=CCNCC4)c(F)c3)o2)ccc1C1=CCNCC1]

Synthesis of 2,5-Bis(3-fluoro-4-(1,2,3,6-tetrahydropyridin-4-yl)phenyl)-1,3,4-oxadiazole: A flask was charged with 490 mg (0.75 mmol) of the from tert-butyl 4,4′-(4,4′-(1,3,4-oxadiazole-2,5-diyl)bis(2-fluoro-4,1-phenylene))bis(5,6-dihydropyridine-1(2H)-carboxylate) which was dissolved with 4 mL dichloromethane. Then 4 mL (52 mmol) of trifluoroacetic acid added over two minutes with stirring. The next day the reaction was concentrated under vacuum and triturated with two 10 mL portions of 7/3 ether/ethyl acetate and solvent decanted away to yield a solid in quantitative yield.

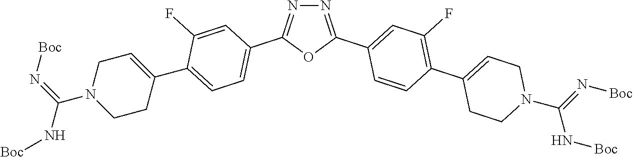 [SMILES: CC(C)(C)OC(=O)/N=C(/NC(=O)OC(C)(C)C)N1CC=C(c2ccc(-c3nnc(-c4ccc(C5=CCN(/C(=N/C(=O)OC(C)(C)C)NC(=O)OC(C)(C)C)CC5)c(F)c4)o3)cc2F)CC1]

Synthesis of tert-Butyl (4,4′-(4,4′-(1,3,4-oxadiazole-2,5-diyl)bis(2-fluoro-4,1-phenylene)) bis(5,6-dihydropyridine-4,1(2H)-diyl))bis(methanetriyl)tetracarbamate: A flask was charged with 312 mg (0.753 mmol) of 2,5-bis(3-fluoro-4-(1,2,3,6-tetrahydropyridin-4-yl)phenyl)-1,3,4-oxadiazole bis trifluoroacetic acid salt, 3 mL of dichloromethane and 2 mL N,N-dimethylformamide. Then 303 mg (100 mmol) of triethylamine added over two minutes. Then 372 mg (1.20 mmol) of tert-butyl (1H-pyrazol-1-yl)methanediylidenedi carbamate was added. The reaction was stirred at 23° C. for four hours. The reaction was concentrated under vacuum, diluted with 30 mL ethyl acetate and then washed with 20 mL of water, 20 mL of 10% citric acid and 20 mL of water. Organic phase was dried with sodium sulfate and concentrated under vacuum, dissolved with 2 mL dichloromethane and 150 ul of 1-methyl piperazine added. The next day the reaction was concentrated under vacuum and purified by normal phase chromatography, loaded as dichloromethane solution, eluted with (20% acetonitrile dichloromethane)/dichloromethane with step gradient from 0 to 100% to elute product. Yield was 150 mg of product (21%).

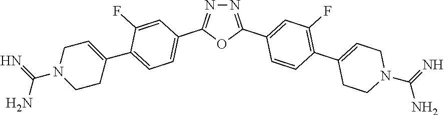 [SMILES: N=C(N)N1CC=C(c2ccc(-c3nnc(-c4ccc(C5=CCN(C(=N)N)CC5)c(F)c4)o3)cc2F)CC1]

### Example 49

Synthesis of 4,4′-(4,4′-(1,3,4-oxadiazole-2,5-diyl)bis(2-fluoro-4,1-phenylene)) bis(5,6-dihydropyridine-1(2H)-carboximidamide): A flask was charged with 150 mg (0.166 mmol) of tert-butyl (4,4′-(4,4′-(1,3,4-oxadiazole-2,5-diyl)bis(2-fluoro-4,1-phenylene))bis(5,6-dihydropyridine-4,1(2H)-diyl))bis(methanetriyl)tetracarbamate which was dissolved with 3 mL of dichloromethane. Then 4 mL (52 mmol) of trifluoroacetic acid added over two minutes with stirring and stirred for one hour and concentrated under vacuum. Oil was triturated with two 20 mL portions of ether and ether decanted away to yield a solid. Yield was 94 mg (77%). ¹H NMR (300 MHz, DMSO-d₆) δ=8.03-7.95 (m, 2H), 7.63 (t, J=8.1 Hz, 2H), 7.43 (s, 6H), 7.51-7.35 (m, 2H), 6.19 (s, 2H), 4.08 (br. d, J=3.4 Hz, 4H), 3.64-3.45 (m, 4H), 2.58 (br s, 4H). LC/MS method A: R_{f}=3.28 mins., (M+H)⁺=506, purity >95%.

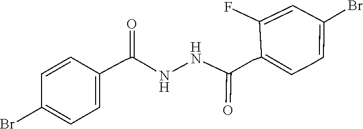 [SMILES: O=C(CNNC(=O)CC1=CCC=C(Br)C=C1F)Cc1ccc(Br)cc1]

Synthesis of 4-Bromo-N′-(4-bromobenzoyl)-2-fluorobenzohydrazide: A flask was charged with 1560 mg (7.24 mmol) of 4-bromobenzohydrazide and slurried with 25 mL of chloroform and 1310 mg (10.1 mmol) of N,N-diisopropylethylamine and cooled in an ice water bath. Then a solution of 4-bromo-2-fluorobenzoyl chloride (1800 mg, 7.60 mmol) in 20 mL chloroform was added dropwise over 40 minutes. The reaction was allowed to stir for 0.18 hours at 23° C. The reaction was concentrated under vacuum and briefly sonicated with 25 mL of water and aged for one hour. Water decanted away and two 70 mL acetonitrile added and removed under vacuum to dry solid. Yield was 2770 mg (95%).

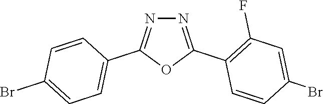 [SMILES: Fc1cc(Br)ccc1-c1nnc(-c2ccc(Br)cc2)o1]

Synthesis of 2-(4-Bromo-2-fluorophenyl)-5-(4-bromophenyl)-1,3,4-oxadiazole: A flask was charged with 2640 mg (6.50 mmol) of 4-bromo-N′44-bromo benzoyl)-2-fluorobenzohydrazide hydrate. The solid was slurried with 20 mL toluene and brief sonicated. Then 4060 mg (19.5 mmol) of PCl₅ was added at once. The reaction was heated to 99° C. for 18 hours. The reaction was cooled and concentrated under vacuum to yield a solid. The reaction was quenched by addition of wet ice (about 30 grams). After 20 minutes later 20 mL water added. After a two hours, the water removed by decantation, solid triturated with 10 mL methanol and the methanol was removed by decantation. Then 30 mL portions of acetonitrile added and removed under vacuum to remove water. Yield 2700 mg of solid (92%).

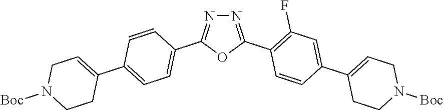 [SMILES: CC(C)(C)OC(=O)N1CC=C(c2ccc(-c3nnc(-c4ccc(C5=CCN(C(=O)OC(C)(C)C)CC5)cc4F)o3)cc2)CC1]

Synthesis of tert-Butyl 4-(4-(5-(4-(1-(tert-butoxycarbonyl)-1,2,3,6-tetrahydropyridin-4-yl)-2-fluorophenyl)-1,3,4-oxadiazol-2-yl)phenyl)-5,6-dihydropyridine-1(2H)-carboxylate: A flask was charged with 400 mg (0.97 mmol) of 2-(4-bromo-2-fluorophenyl)-5-(4-bromophenyl)-1,3,4-oxadiazole, 17 mg (0.078 mmol) of palladium acetate, 80 mg (0.194 mmol) of 2-Dicyclohexylphosphino-2′,6′-dimethoxybiphenyl, 723 mg (2.34 mmol) of tert-butyl 4-(4,4,5,5-tetramethyl-1,3,2-dioxaborolan-2-yl)-5,6-dihydro pyridine-1(2H)-carboxylate, 7 mL of dioxane and 3 mL of 2.0 molar potassium carbonate aqueous solution. The flask was swept with nitrogen for five minutes and heated at 95° C. for 18 hours. The reaction was complete by LCMS and the reaction was allowed to cool and diluted with 10 mL water and 25 mL of ethyl acetate added over two minutes. Solids formed quickly and reaction stirred for two hours. Solid collected on a filter and rinsed with 15 mL ethyl acetate. Yield was 450 mg (77%).

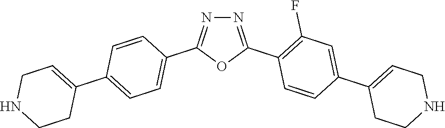 [SMILES: Fc1cc(C2=CCNCC2)ccc1-c1nnc(-c2ccc(C3=CCNCC3)cc2)o1]

Synthesis of 2-(2-Fluoro-4-(1,2,3,6-tetrahydropyridin-4-yl)phenyl)-5-(4-(1,2,3,6-tetrahydro pyridin-4-yl)phenyl)-1,3,4-oxadiaze A flask was charged with the 450 mg (0.74 mmol) tert-butyl 4-(4-(5-(4-(1-(tert-butoxycarbonyl)-1,2,3,6-tetrahydropyridin-4-yl)-2-fluorophenyl)-1,3,4-oxadiazol-2-yl)phenyl)-5,6-dihydropyridine-1(2H)-carboxylate and was dissolved with 6 mL dichloromethane. Then 10 mL (130 mmol) of trifluoroacetic acid added over three minutes with stirring. The reaction was stirred for 2 hours, then was concentrated under vacuum and triturated with two 50 mL portions of ether and ether decanted away to yield a solid in quantitative yield.

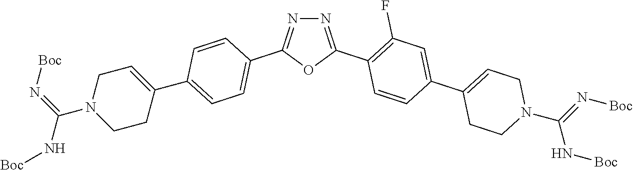 [SMILES: CC(C)(C)OC(=O)/N=C(/NC(=O)OC(C)(C)C)N1CC=C(c2ccc(-c3nnc(-c4ccc(C5=CCN(/C(=N/C(=O)OC(C)(C)C)NC(=O)OC(C)(C)C)CC5)cc4F)o3)cc2)CC1]

Synthesis of tert-Butyl N—[(Z)-(4-{4-[5-(4-{1-[(E)-{[(tert-butoxy)carbonyl] amino}({[(tert-butoxy)carbonyl]imino})methyl]-1,2,3,6-tetrahydropyridin-4-yl}-2-fluoro phenyl)-1,3,4-oxadiazol-2-yl]phenyl}-1,2,3,6-tetrahydropyridin-1-yl)({[(tert-butoxy) carbonyl]imino}) methyl]carbamate: A flask was charged with 450 mg (0.70 mmol) of 2-(2-fluoro-4-(1,2,3,6-tetrahydropyridin-4-yl)phenyl)-5-(4-(1,2,3,6-tetrahydropyridin-4-yl)phenyl)-1,3,4-oxadiazole bis trifluoroacetic acid salt and 5 mL N,N-dimethylformamide. Then 495 mg (4.90 mmol) of triethylamine added over two minutes. Then 52.1 mg (1.68 mmol) of tert-butyl (1H-pyrazol-1-yl)methanediylidenedicarbamate was added. The reaction was stirred at 23° C. for 18 hours. Then 300 mg (0.97 mmol) of tert-butyl (1H-pyrazol-1-yl)methanediylidenedicarbamate was added. After 24 hours, 50 mL, of ethyl acetate was added and washed with three portions of 40 mL of water. The organic phase was dried with sodium sulfate and concentrated under vacuum to yield 690 mg of product in a quantitative yield.

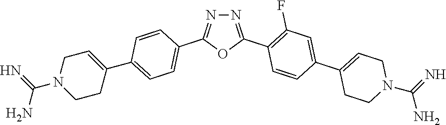 [SMILES: N=C(N)N1CC=C(c2ccc(-c3nnc(-c4ccc(C5=CCN(C(=N)N)CC5)cc4F)o3)cc2)CC1]

### Example 50

Synthesis of 4-(4-(5-(4-(1-Carbamimidoyl-1,2,3,6-tetrahydropyridin-4-yl)-2-fluorophenyl)-1,3,4-oxadiazol-2-yl)phenyl)-5,6-dihydropyridine-1(2H)-carboximidamide: A flask was charged with 690 mg (070 mmol) of tert-butyl N—[(Z)-(4-{4-[5-(4-{1-[(E)-{[(tert-butoxy) carbonyl]amino}({[(tert-butoxy)carbonyl]imino})methyl]-1,2,3,6-tetrahydropyridin-4-yl}-2-fluoro phenyl)-1,3,4-oxadiazol-2-yl]phenyl}-1,2,3,6-tetrahydro pyridin-1-yl)({[(tert-butoxy)carbonyl]imino})methyl]carbamate and was dissolved with a mixture of 400 mg (3.70 mmol) of anisole and 6 mL of dichloromethane. Then 7 mL (90.9 mmol) of trifluoroacetic acid added over four minutes with stirring and stirred for 18 hours. The LCMS indicted reaction complete and reaction concentrated under vacuum to yield a solid. Solid was dissolved in N,N-dimethylformamide and was purified by prep HPLC. Yield was 8 mg (1.6%). ¹H NMR (300 MHz, DMSO-d₆) δ=8.19-8.04 (m, 4H), 7.77-7.52 (m, 4H), 7.52-7.40 (m, 7H), 6.54 (dd, J=0.5, 2.7 Hz, 1H), 6.43 (br t, J=2.3 Hz, 1H), 4.17-4.08 (m, 2H), 3.68-3.60 (m, 2H), 3.27-2.95 (m, 2H), 2.87-2.60 (m, 2H), 2.60-2.54 (m, 2H). LC/MS method A: R_{f}=3.05 mins., (M+H)⁺=487,244, purity >90%.

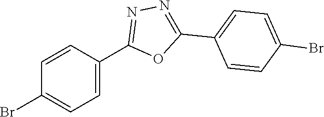 [SMILES: Brc1ccc(-c2nnc(-c3ccc(Br)cc3)o2)cc1]

2,5-Bis(4-bromophenyl)-1,3,4-oxadiazole was prepared from (1,N)-4-Bromo-N′-((4-bromophenyl)chloromethylene)benzohydrazonoyl chloride and phosphorus oxychloride.

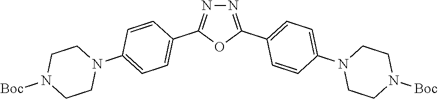 [SMILES: CC(C)(C)OC(=O)N1CCN(c2ccc(-c3nnc(-c4ccc(N5CCN(C(=O)OC(C)(C)C)CC5)cc4)o3)cc2)CC1]

Synthesis of tert-Butyl 4,4′-(4,4′-(1,3,4-oxadiazole-2,5-diyl)bis(4,1-phenylene))-dipiperazine-1-carboxylate: A flask was charged with 193 mg (0.508 mmol) of 2,5-bis(4-bromophenyl)-1,3,4-oxadiazole, 9 mg (0.041 mmol) of palladium acetate, 42 mg (0.102) of 2-dicyclohexylphosphino-2′,6′-dimethoxybiphenyl, 391 mg (2.10 mmol) of 1-Bocpiperazine, 3 mL of dioxane and 828 mg of cesium carbonate. The flask was swept with nitrogen for five minutes and heated at 95° C. for 18 hours. The reaction was complete by LCMS and the reaction was allowed to cool and diluted with 15 mL water and 10 mL of ethyl acetate was added over two minutes. Solids formed quickly and reaction stirred for 30 minutes. Solid collected on a filter and air-dried for 18 hours in the hood to yield 360 mg (100%) plus additional weight from ligand, solvent or inorganics.

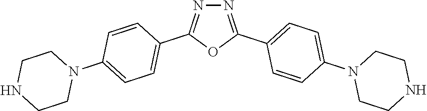 [SMILES: c1cc(N2CCNCC2)ccc1-c1nnc(-c2ccc(N3CCNCC3)cc2)o1]

Synthesis of 2,5-Bis(4-(piperazin-1-yl)phenyl)-1,3,4-oxadiazole: A flask was charged with 360 mg (0.508 mmol) of the tert-butyl 4,4′-(4,4′-(1,3,4-oxadiazole-2,5-diyl)bis(4,1-phenylene))dipiperazine-1-carboxylate which was slurried with 3 mL dichloromethane. Then 6 mL (98 mmol) of trifluoroacetic acid added over five minutes with stirring. Two hours later the reaction was concentrated under vacuum and triturated with two 20 mL portions of ether and ether decanted away to yield a solid in quantitative yield.

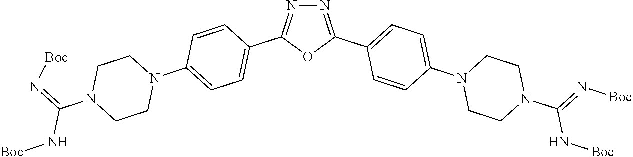 [SMILES: CC(C)(C)OC(=O)/N=C(/NC(=O)OC(C)(C)C)N1CCN(c2ccc(-c3nnc(-c4ccc(N5CCN(/C(=N/C(=O)OC(C)(C)C)NC(=O)OC(C)(C)C)CC5)cc4)o3)cc2)CC1]

Synthesis of tert-Butyl (4,4′-(4,4′-(1,3,4-oxadiazole-2,5-diyl)bis(4,1-phenylene))bis (piperazine-4,1-diyl))bis-(methanetriyl)tetracarbamate: A flask was charged with 310 mg (0.508 mmol) of 2,5-bis(4-(piperazin-1-yl)phenyl)-1,3,4-oxadiazole bis trifluoroacetic acid salt and 4 mL dichloromethane. Then 350 mg (3.46 mmol) of triethylamine was added over one minute. Then 326 mg (39.4 mmol) of tert-butyl (1H-pyrazol-1-yl)methanediylidenedicarbamate was added. The reaction slurry was stirred at 23° C. for 18 hours. The reaction was concentrated under vacuum, diluted with 15 mL ethyl acetate and then washed with 15 mL of water. Organic phase was dried with sodium sulfate and concentrated under vacuum. The product was purified by normal phase chromatography, loaded as dichloromethane solution, eluted with (15% isopropanol/dichloromethane)/dichloromethane with step gradient from 0 to 80% to elute product. Yield was 380 mg of product (86%).

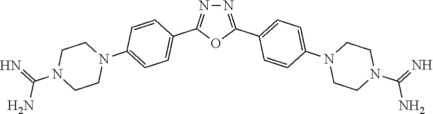 [SMILES: N=C(N)N1CCN(c2ccc(-c3nnc(-c4ccc(N5CCN(C(=N)N)CC5)cc4)o3)cc2)CC1]

### Example 51

Synthesis of 4,4′-(4,4′-(1,3,4-Oxadiazole-2,5-diyl)bis(4,1-phenylene)) dipiperazine-1-carboximidamide: A flask was charged with 380 mg (0.435 mmol) of tert-butyl (4,4′-(4,4′-(1,3,4-oxadiazole-2,5-diyl)bis(4,1-phenylene))bis(piperazine-4,1-diyl))bis-(methanetriyl)tetra carbamate which was dissolved with 3 mL of dichloromethane. Then 4 mL (52 mmol) of trifluoroacetic acid added over two minutes with stirring and stirred for 3 hours. Then the reaction was concentrated under vacuum. The concentrate was slurried with 20% acetonitrile/water, frozen and freeze dried. Yield was 252 mg (83%). ¹H NMR (300 MHz, DMSO-d₆) δ=8.85 (br d, J=9.0 Hz, 2H), 8.85 (br s, 2H), 7.67 (d, J=8.8 Hz, 4H), 7.67 (d, J=8.8 Hz, 4H), 7.19 (d, J=9.0 Hz, 2H), 3.70 (s, 2H), 3.59-3.41 (m, 6H), 2.46-2.11 (m, 2H). LC/MS method A: R_{f}=2.70 mins., (M+H)⁺=475, purity >95%.

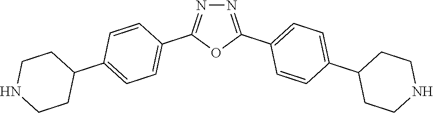 [SMILES: c1cc(C2CCNCC2)ccc1-c1nnc(-c2ccc(C3CCNCC3)cc2)o1]

Synthesis of 2,5-Bis(4-(piperidin-4-yl)phenyl)-1,3,4-oxadiazole: A of 4-(4-{5-[4-(1,2,3,6-tetrahydropyridin-4-yl)phenyl]-1,3,4-oxadiazol-2-yl}phenyl)-1,2,3,6-tetrahydropyridine (0.10 g, 0.16 mmol) was hydrogenated in methanol (3 mL) over 5% palladium on carbon (20 mg) at 45 psi initial hydrogen pressure for 18 hours. The catalyst was filtered and the solvents evaporated to leave the product as a crystalline solid (100 mg, 100%). 1H-NMR (CD3OD) δ 8.13 (d, 4H, J==8.2 Hz), 7.54 (d, 4H, J=8.2 Hz), 3.57 (m, 4H), 3.18 (td, 4H, J=122.9 Hz, J=2.9 Hz), 3.05 (tt, 2H, J=12.0 Hz, J=3.5 Hz), 2.15 (m, 4H), 1.95 (m, 4H). LCMS method A R_{f}=2.74 mins, purity >95%, (M+H)+=389.

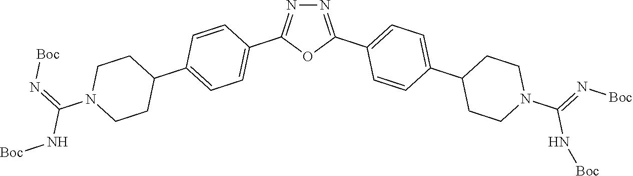 [SMILES: CC(C)(C)OC(=O)/N=C(/NC(=O)OC(C)(C)C)N1CCC(c2ccc(-c3nnc(-c4ccc(C5CCN(/C(=N/C(=O)OC(C)(C)C)NC(=O)OC(C)(C)C)CC5)cc4)o3)cc2)CC1]

tert-Butyl (4,4′-(4,4′-(1,3,4-oxadiazole-2,5-diyl)bis(4,1-phenylene))bis (piperidine-4,1-diyl))bis(methanetriyl)tetracarbamate was prepared from 2,5-bis(4-(piperidin-4-yl)phenyl)-1,3,4-oxadiazole.

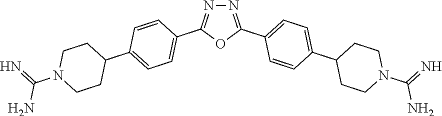 [SMILES: N=C(N)N1CCC(c2ccc(-c3nnc(-c4ccc(C5CCN(C(=N)N)CC5)cc4)o3)cc2)CC1]

### Example 52

Synthesis of 4,4′-(4,4′-(1,3,4-Oxadiazole-2,5-diyl)bis(4,1-phenylene)) dipiperidine-1-carboximidamide: A solution of 4-(4-{5-[4-(piperidin-4-yl)phenyl]-1,3,4-oxadiazol-2-yl}phenyl)piperidine (30 mg, 49 μmol), N—((N′,N″-bis-tert-butyloxy carbonyl)amidino)pyrrazole (45 mg, 0.15 mmol) and N,N-diisopropylethylamine (40 mg, 0.39 mmol, 54 μl) in methanol (0.5 mL) was stirred for 18 hours. The product was purified by preparative reverse phase HPLC and the product fractions were evaporated in vacuo. The crude product was dissolved in trifluoroacetic acid (1 mL) and dichloromethane (1 mL) and stirred for 2 hours then evaporated to dryness. The product was dissolved in water (3 mL) and acetonitrile (1 mL) and lyophilized to leave a beige powder (23 mg, 67%). 1H-NMR (CD3OD) δ 8.10 (d, 4H, J:=: 8.5 Hz), 7.53 (d, 4H, J=8.5 Hz), 4.03 (m, 4H), 3.22 (m, 6H), 3.02 (m, 2H), 2.01 (m, 4H), 1.80 (m, 4H). LCMS method A R_{f}=3.20 mins, purity >95%, (M+H)+=473.

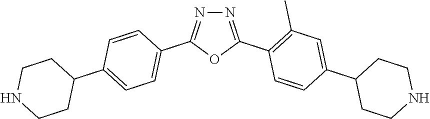 [SMILES: Cc1cc(C2CCNCC2)ccc1-c1nnc(-c2ccc(C3CCNCC3)cc2)o1]

Synthesis of 2-(2-Methyl-4-(piperidin-4-yl)phenyl)-5-(4-(piperidin-4-yl)phenyl)-1,3,4-oxadiazole: A solution of tert-butyl 4-{4-[5-(4-{1-[(tert-butoxy)carbonyl]piperidin-4-yl}-2-methylphenyl)-1,3,4-oxadiazol-2-yl]phenyl}piperidine-1-carboxylate (115 mg, 0.19 mmol) in trifluoroacetic acid (1 mL) and dichloromethane (1 mL) was stirred for 2 hours and evaporated. The product was lyophilized to leave a yellow foamy solid (132 mg, 93%).

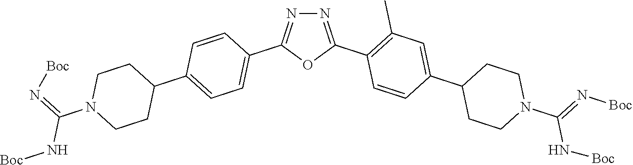 [SMILES: Cc1cc(C2CCN(/C(=N/C(=O)OC(C)(C)C)NC(=O)OC(C)(C)C)CC2)ccc1-c1nnc(-c2ccc(C3CCN(/C(=N\C(=O)OC(C)(C)C)NC(=O)OC(C)(C)C)CC3)cc2)o1]

tert-Butyl N-[(E)-(4-{4-[5-(4-{1-[(Z)-{[(tert-Butoxy)carbonyl]amino}({[(tert-butoxy)carbonyl]imino})methyl]piperidin-4-yl}phenyl)-1,3,4-oxadiazol-2-yl]-3-methyl phenyl}piperidin-1-yl)({[(tert-butoxy)carbonyl]imino})methyl]carbamate was prepared from 2-(2-methyl-4-(piperidin-4-yl)phenyl)-5-(4-(piperidin-4-yl)phenyl)-1,3,4-oxadiazole.

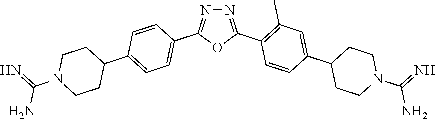 [SMILES: Cc1cc(C2CCN(C(=N)N)CC2)ccc1-c1nnc(-c2ccc(C3CCN(C(=N)N)CC3)cc2)o1]

### Example 53

4-(4-(5-(4-(1-Carbamimidoylpiperidin-4-yl)-2-methylphenyl)-1,3,4-oxadiazol-2-yl)phenyl)piperidine-1-carboximidamide: A solution of 4-(4-{5-[2-methyl-4-(piperidin-4-yl)phenyl]-1,3,4-oxadiazol-2-yl}phenyl)piperidine (125 mg, 0.17 mmol), N—((N′,N″-bis-tert-butyloxycarbonyl)amidino)pyrrazole (156 mg, 0.50 mmol) and N,N-(diisopropyl)ethylamine (219 mg, 1.7 mmol, 305 μl) in methanol (2 mL) was stirred for 18 hours. The solvent was evaporated and the crude product was purified by reverse phase HPLC. The product fractions were combined, treated with saturated sodium bicarbonate solution, extracted with dichloromethane (2×50 mL), dried and evaporated to leave product (150 mg, 100%) as a glassy solid. The product was dissolved in trifluoroacetic acid (2 mL) and dichloromethane (2 mL). After stirring for 2 hours the solvents were evaporated, the crude product was purified by preparative reverse phase HPLC and the product fractions were lyophilized to leave a beige solid (91 mg, 65%). ¹H NMR (300 MHz, METHANOL-d₄) δ=8.16-8.00 (m, 4H), 7.73-7.46 (m, 2H), 7.43-7.27 (m, 1H), 4.19-3.90 (m, 4H), 3.28-3.16 (m, 4H), 3.14-2.90 (m, 1H), 2.73 (s, 3H), 2.78-2.71 (m, 1H), 2.16-1.93 (m, 4H), 1.90-1.73 (m, 4H). LC/MS method A: R_{f}=3.09 mins., (M+H)⁺=487, purity >90%.

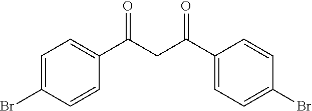 [SMILES: O=C(CC(=O)c1ccc(Br)cc1)c1ccc(Br)cc1]

Synthesis of 1,3-Bis(4-bromophenyl)propane-1,3-dione (intermediate E). A flask was charged with 9100 mg (46.1 mmol) of 1-(4-bromophenyl)ethanone and 100 mL dry tetrahydrofuran. Then 2700 mg (57.5 mmol) equivalents of 60% sodium hydride added in portions and reaction warmed to 45° C. and a solution ethyl 4-bromobenzoate (11100 mg, 48.5 mmol) in 5 ml, of tetrahydrofuran was added dropwise over two minutes and reaction temp was heated to 60° C. for four hours. The reaction was allowed to cool and the reaction was quenched by addition of 4 mL of methanol added over five minutes. Then 20 mL equivalents of 3.0 N HCl was added over 20 minutes and solids formed. After one hour, solids were collected, air dried to yield 11.9 grams. A second and third crop of crystals were collected from mother liquor and dried to yield a total of 17.4 grams

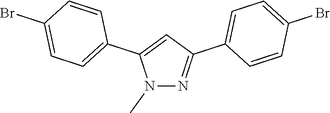 [SMILES: Cn1nc(-c2ccc(Br)cc2)cc1-c1ccc(Br)cc1]

Synthesis of 3,5-Bis(4-bromophenyl)-1-methyl-1H-pyrazole: A flask was charged with 2080 mg (5.44 mmol) of 1,3-bis(4-bromophenyl)propane—in 25 mL ethanol (denatured with alcohol). Then 128 mg (3.2 mmol) of acetic acid added followed by addition of methylhydrazine (300 mg, 6.53 mmol) The reaction is heated at 65° C. for 18 hours. After cooling, solid collected and air dried for 18 hours. Yield was 550 mg (51%).

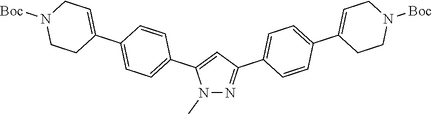 [SMILES: Cn1nc(-c2ccc(C3=CCN(C(=O)OC(C)(C)C)CC3)cc2)cc1-c1ccc(C2=CCN(C(=O)OC(C)(C)C)CC2)cc1]

Synthesis of tert-Butyl 4,4′-(4,4′-(1-methyl-1H-pyrazole-3,5-diyl)bis(4,1-phenylene))bis (5,6-dihydropyridine-1(2H)-carboxylate): A flask was charged with 356 mg (0.908 mmol) of 3,5-bis(4-bromophenyl)-1-methyl-1H-pyrazole and tert-butyl 4-(4,4,5,5-tetramethyl-1,3,2-dioxaborolan-2-yl)-5,6-dihydropyridine-1(2H)-carboxylate, 14 mg (0.064 mmol) of palladium acetate, 67 mg (0.163 mmol) of 2-dicyclohexylphosphino-2′,6′-dimethoxybiphenyl, 641 mg (2.08 mmol) of tert-butyl 4-(4,4,5,5-tetramethyl-1,3,2-dioxaborolan-2-yl)-5,6-dihydropyridine-1(2H)-carboxylate, 6 mL of dioxane and 3.2 mL of 2.0 molar potassium carbonate aqueous solution. The flask was swept with nitrogen for five minutes and heated at 95° C. for 18 hours. The reaction was complete by LCMS and the reaction was allowed to cool and diluted with 10 mL water and 25 mL of ethyl acetate added over two minutes. Both phases filtered thru celite and organic phase was separated and concentrated under vacuum. The concentrate was chromatographed on silica using ethyl acetate/dichloromethane step gradient from 0 to 100%. Yield was 440 mg of an oil (81%).

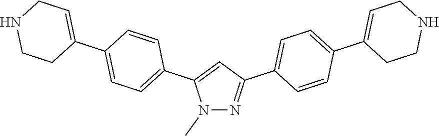 [SMILES: Cn1nc(-c2ccc(C3=CCNCC3)cc2)cc1-c1ccc(C2=CCNCC2)cc1]

Synthesis of 4,4′-(4,4′-(1-Methyl-1H-pyrazole-3,5-diyl)bis(4,1-phenylene)) bis(1,2,3,6-tetrahydropyridine: A flask was charged with 440 mg (0.737 mmol) of the tert-butyl 4,4′-(4,4′-(1-methyl-1H-pyrazole-3,5-diyl)bis(4,1-phenylene))bis(5,6-dihydropyridine-1(2H)-carboxylate) was slurried with 4 mL dichloromethane. Then 6 mL (96 mmol) of trifluoroacetic acid added over two minutes with stirring. After 24 hours the reaction was concentrated under vacuum and triturated with 20 mL portions of ether and solid collected in quantitative yield.

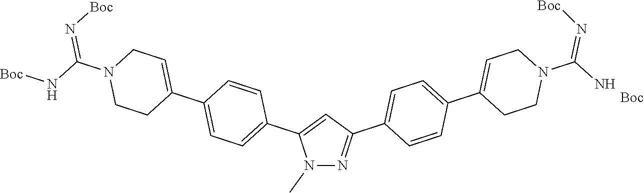 [SMILES: Cn1nc(-c2ccc(C3=CCN(/C(N)=N\C(=O)OC(C)(C)C)CC3)cc2)cc1-c1ccc(C2=CCN(/C(=N\C(=O)OC(C)(C)C)NC(=O)OC(C)(C)C)CC2)cc1 ; "Boc"]

Synthesis of tert-Butyl(4,4′-(4,4′-(1-methyl-1H-pyrazole-3,5-diyl)bis(4,1-phenylene)) bis(5,6-dihydropyridine-4,1(2H)-diyl))bis(methanetriyl)tetracarbamate: A flask was charged with 460 mg (0.737 mmol) of 4,4′-(4,4′-(1-methyl-1H-pyrazole-3,5-diyl)bis(4,1-phenylene))bis(1,2,3,6-tetrahydropyridine bis trifluoroacetic acid salt, 4 mL dichloromethane and 2 mL N,N-dimethylformamide. Then 490 mg (4.85 mmol) of triethylamine added over two minutes. Then 260 mg (0.84 mmol) of tert-butyl (1H-pyrazol-1-yl)methanediylidene dicarbamate was added. The reaction was stirred at 23° C. for 18 hours. The reaction was concentrated under vacuum and purified by normal phase chromatography, loaded as dichloromethane solution, eluted with ethyl acetate/dichloromethane step gradient from 0 to 100% to elute product. Yield was 460 mg of product (71%).

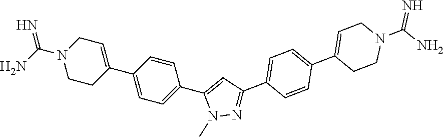 [SMILES: Cn1nc(-c2ccc(C3=CCN(C(=N)N)CC3)cc2)cc1-c1ccc(C2=CCN(C(=N)N)CC2)cc1]

### Example 54

Synthesis of 4,4′-(4,4′-(1-Methyl-1H-pyrazole-3,5-diyl)bis(4,1-phenylene)) bis(5,6-dihydropyridine-1(2H)-carboximidamide): A flask was charged with 230 mg (0.261 mmol) of tert-Butyl (4,4′-(4,4′-(1-methyl-1H-pyrazole-3,5-diyl)bis(4,1-phenylene))bis(5,6-dihydropyridine-4,1(2H)-diyl))bis(methanetriyl)tetracarbamate which was dissolved with 3 mL of dichloromethane. Then 3 mL (39 mmol) of trifluoroacetic acid added over two minutes with stirring and stirred for two hours. The reaction was concentrated under vacuum. Oil was triturated with two 20 mL portions of ether and ether decanted away to yield a solid. Solid was slurried with 20% acetonitrile/water, frozen and freeze dried to yield 120 mg (65%). ¹H NMR (300 MHz, DMSO-d₆) δ=7.80 (d, J=8.5 Hz, 1H), 7.66-7.34 (m, 6H), 6.90 (s, 2H), 6.27 (br d, J=19.5 Hz, 2H), 4.07 (br dd, J=3.4, 5.6 Hz, 2H), 3.88 (s, 3H), 3.75-3.49 (m, 2H), 3.49-3.39 (m, 2H), 3.27-2.99 (m, 2H), 2.69-2.49 (m, 2H), 2.43-2.23 (m, 2H). LC/MS method A: R_{f}=2.97 mins., (M+H)⁺=481, purity >95%.

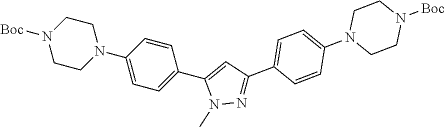 [SMILES: Cn1nc(-c2ccc(N3CCN(C(=O)OC(C)(C)C)CC3)cc2)cc1-c1ccc(N2CCN(C(=O)OC(C)(C)C)CC2)cc1]

Synthesis of tert-Butyl. (4,4′-(4,4′-(1-methyl-1H-pyrazole-3,5-diyl)bis(4,1-phenylene))bis (piperazine-4,1-diyl))bis(methanetriyl)tetracarbamate: A flask was charged with 186 mg (0.478 mmol) of 3,5-Bis(4-bromophenyl)-1-methyl-1H-pyrazole, 18 mg (0.071 mmol) of palladium acetate, 25 mg (0.062) of 2-Dicyclohexylphosphino-2′,6′-dimethoxy biphenyl, 172 mg (0.924 mmol) of 1-Bocpiperazine, 3 mL of dioxane and 603 mg (1.85 mmol) of cesium carbonate. The flask was swept with nitrogen for five minutes and heated at 95° C. for 18 hours. The reaction was complete by LCMS and the reaction was allowed to cool and diluted with 10 mL, water and 10 mL of ethyl acetate was added over two minutes. Solids formed quickly and reaction stirred for 30 minutes. Solid collected on a filter and air-dried for 30 minutes in the hood plus mass from organic layer to yield 320 mg (100%) plus weight of solvent and ligand.

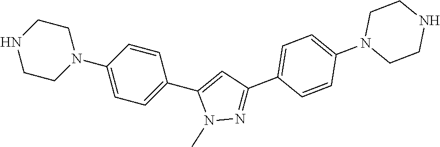 [SMILES: Cn1nc(-c2ccc(N3CCNCC3)cc2)cc1-c1ccc(N2CCNCC2)cc1]

Synthesis of 1,1′-(4,4′-(1-Methyl-1H-pyrazole-3,5-diyl)bis(4,1-phenylene)) dipiperazine: flask was charged with 320 mg (0.503 mmol) of the tert-butyl. (4,4′-(4,4′-(1-methyl-1H-pyrazole-3,5-diyl)bis(4,1-phenylene))bis(piperazine-4,1-diyl))bis(methanetriyl) tetracarbamate which was slurried with 5 mL of dichloromethane. Then 8 mL (104 mmol) of trifluoroacetic acid added over five minutes with stirring. After four hours the reaction was concentrated under vacuum and triturated with two 30 mL portions of ether and ether decanted away to yield 240 mg of a solid. (yield 75%).

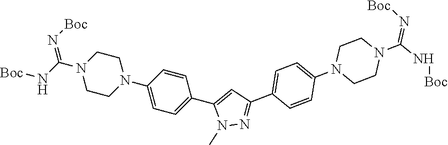 [SMILES: Cn1nc(-c2ccc(N3CCN(/C(=N\C(=O)OC(C)(C)C)NC(=O)OC(C)(C)C)CC3)cc2)cc1-c1ccc(N2CCN(/C(=N\C(=O)OC(C)(C)C)NC(=O)OC(C)(C)C)CC2)cc1]

Synthesis of tert-Butyl (4,4′-(4,4′-(1-methyl-1H-pyrazole-3,5-diyl)bis(4,1-phenylene))bis (piperazine-4,1-diyl))bis(methanetriyl)tetracarbamate: A flask was charged with 180 mg (0.280 mmol) of 1,1′-(4,4′-(1-methyl-1H-pyrazole-3,5-diyl)bis(4,1-phenylene)) dipiperazine bis trifluoroacetic acid salt and 4 mL dichloromethane. Then 260 mg (2.52 mmol) of triethylamine added over two minutes. Then 217 mg (0.810 mmol) of tert-butyl (1H-pyrazol-1-yl)methanediylidenedicarbamate was added. After four hours an additional 330 mg (1.06 mmol) of tert-butyl (1H-pyrazol-1-yl)methanediylidenedicarbamate was added and reaction was stirred for 18 hours at 23° C. An additional 150 mg (0.483 mmol) of tert-butyl (1H-pyrazol-1-yl)methanediylidenedicarbamate was added. Four hours later the reaction was concentrated under vacuum and reaction was diluted with 10 mL ethyl acetate and 10 mL of water and stirred for 30 minutes. The organic phase was filtered and dried with sodium sulfate and concentrated under vacuum. The product was purified by normal phase chromatography, loaded as dichloromethane solution, eluted with (25% isopropanol/dichloromethane with step gradient from 0 to 100% to elute product. Then chromatographed a second time with (12% isopropanol/dichloromethane with step gradient from 0 to 50% to elute product. Yield was 250 mg of product (99%).

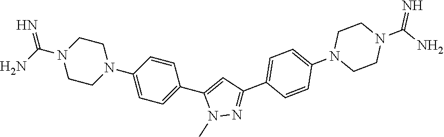 [SMILES: Cn1nc(-c2ccc(N3CCN(C(=N)N)CC3)cc2)cc1-c1ccc(N2CCN(C(=N)N)CC2)cc1]

### Example 55

Synthesis of 4,4′-(4,4′-(1-Methyl-1H-pyrazole-3,5-diyl)bis(4,1-phenylene)) dipiperazine-1-carboximidamide: A flask was charged with 250 mg (0.280 mmol) of tert-butyl(4,4′-(4,4′-(1-methyl-1H-pyrazole-3,5-diyl)bis(4,1-phenylene))bis(piperazine-4,1-diyl))bis (methanetriyl)tetracarbamate which was dissolved with 4 mL of dichloromethane. Then 6 mL (77.9 mmol) of trifluoroacetic acid added over two minutes with stirring and stirred for 18 hours. LCMS indicted reaction complete and the reaction was concentrated under vacuum. Oil was triturated with two 30 portions of ether and ether decanted away to yield a solid. Solid was dissolved in N,N-dimethylformamide and was purified by prep HPLC. Yield was 144 mg (72%). ¹H NMR (300 MHz, DMSO-d₆) δ=9.12 (br t, J=9.4 Hz, 4H), 8.85 (br dd, J==9.5, 19.3 Hz, 2H), 7.69 (br d, J=7.9 Hz, 2H), 7.45 (d, J=8.4 Hz, 2H), 7.12-7.06 (m, 2H), 7.01 (br d, J=4.8 Hz, 2H), 6.67 (s, 1H), 3.83-3.50 (m, 6H), 3.49-3.33 (m, 3H), 3.27-3.13 (m, 2H), 3.12-3.02 (m, 2H), 2.45-2.34 (m, 2H). LC/MS method A: R_{f}=2.72 mins., (M+H)⁺=487, purity >95%.

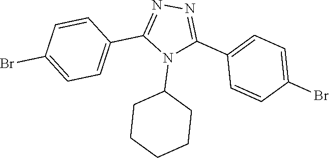 [SMILES: Brc1ccc(-c2nnc(-c3ccc(Br)cc3)n2C2CCCCC2)cc1]

Synthesis of 3,5-Bis(4-bromophenyl)-4-cyclohexyl-4H-1,2,4-triazole A flask was charged with (Z)-4-bromo-N-[(1Z)-(4-bromo-2-fluorophenyl)(chloro)methylidene]-2-fluorobenzene-1-carbohydrazonoyl chloride (1240 mg, 2.87 mmol) which was slurried with 5 mL of methanol and 5 mL N,N-dimethylformamide. The cyclohexylamine (653 mg, 6.60 mmol) was added over two minutes. Then 1100 mg (8.61 mmol) of diisopropylethylamine was added over 15 minutes. After 30 minutes the reaction was slowly warmed to 40° C. for two hours and 50° C. for 18 hours. The reaction was cooled and 5.0 grams (83 mmol) of glacial acetic acid was added over two minutes. The reaction was heated at 80° C. for 72 hours. The reaction was allowed to cool and most of methanol removed under vacuum. Then 20 mL of water added over several minutes. One hour later the solid was collected, rinsed with 10 mL water, air dried for 15 minutes and rinsed with ten mL of ether and air dried in the hood for several hours. Yield was 1.30 grams (98%).

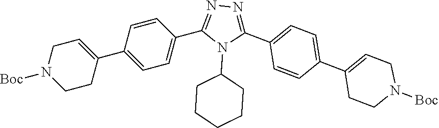 [SMILES: CC(C)(C)OC(=O)N1CC=C(c2ccc(-c3nnc(-c4ccc(C5=CCN(C(=O)OC(C)(C)C)CC5)cc4)n3C3CCCCC3)cc2)CC1]

Synthesis of tert-Butyl 4,4′-(4,4′-(4-cyclohexyl-4H-1,2,4-triazole-3,5-diyl)bis(4,1-phenylene))bis(5,6-dihydropyridine-1(2H)-carboxylate): A flask was charged with 830 mg (1.80 mmol) of 3,5-bis(4-bromophenyl)-4-cyclohexyl-4H-1,2,4-triazole and tert-butyl 4-(4,4,5,5-tetramethyl-1,3,2-dioxaborolan-2-yl)-5,6-dihydropyridine-1(2H)-carboxylate, 32 mg (0.144 mmol) of palladium acetate, 148 mg (0.36 mmol) of 2-dicyclohexylphosphino-2′,6′-dimethoxybiphenyl, 1350 mg (4.32 mmol) of tert-butyl 4-(4,4,5,5-tetramethyl-1,3,2-dioxaborolan-2-yl)-5,6-dihydropyridine-1(2H)-carboxylate, 11 mL of dioxane and 7.0 mL of 2.0 molar potassium carbonate aqueous solution. The flask was swept with nitrogen for five minutes and heated at 95° C. for 18 hours. The reaction was complete by LCMS and the reaction was allowed to cool and diluted with 15 mL water and 40 mL of ethyl acetate added over two minutes. Solids formed quickly and reaction stirred for two hours. Solid collected on a filter, rinsed with 10 mL of ether and air-dried for 18 hours in the hood to yield 700 mg (58%).

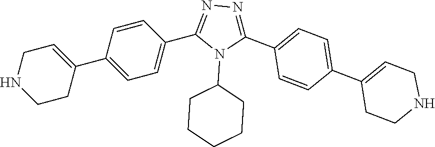 [SMILES: C1=C(c2ccc(-c3nnc(-c4ccc(C5=CCNCC5)cc4)n3C3CCCCC3)cc2)CCNC1]

Synthesis of 4,4′-(4,4′-(4-Cyclohexyl-4H-1,2,4-triazole-3,5-diyl)bis(4,1-phenylene))bis (1,2,3,6-tetrahydropyridine) A flask was charged with 700 mg (1.05 mmol) of the tert-butyl 4,4′-(4,4′-(4-cyclohexyl-4H-1,2,4-triazole-3,5-diyl)bis(4,1-phenylene))bis(5,6-dihydropyridine-1(2H)-carboxylate) was slurried with 1000 mg (9.26 mmol) of anisole and 8 mL dichloromethane. Then 10 mL (130 mmol) of trifluoroacetic acid added over five minutes with stirring. One hour later the reaction was concentrated under vacuum and triturated with two 20 mL portions of ether and ether decanted away to yield a solid in quantitative yield.

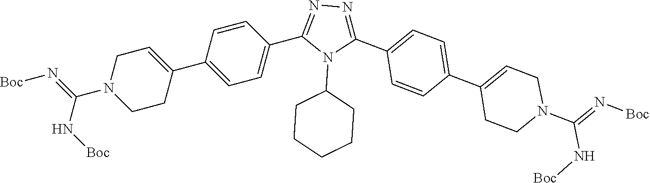 [SMILES: CC(C)(C)OC(=O)/N=C(\NC(=O)OC(C)(C)C)N1CC=C(c2ccc(-c3nnc(-c4ccc(C5=CCN(/C(=N/C(=O)OC(C)(C)C)NC(=O)OC(C)(C)C)CC5)cc4)n3C3CCCCC3)cc2)CC1]

Synthesis of tert-Butyl (4,4′-(4,4′-(4-cyclohexyl-4H-1,2,4-triazole-3,5-diyl)bis(4,1-phenylene))bis(5,6-dihydropyridine-4,1(2H)-diyl))bis(methanetriyl)tetra carbamate: A flask was charged with 570 mg (0.82 mmol) of 4,4′-(4,4′-(4-cyclohexyl-4H-1,2,4-triazole-3,5-diyl)bis(4,1-phenylene))bis(1,2,3,6-tetrahydropyridine) bis trifluoroacetic acid salt, 2 mL of dichloromethane and 8 mL N,N-dimethylformamide. Then 920 mg (9.1 mmol) of triethylamine added over five minutes. Then 970 mg (3.12 mmol) of tert-butyl (1H-pyrazol-1-yl)methanediylidenedicarbamate was added. The reaction slurry was warmed to 50° C. for 18 hours. The reaction was diluted with 30 mL ethyl acetate and then washed with three portions of 20 mL of water. Organic phase was dried with sodium sulfate and concentrated under vacuum to yield product in quantitative yield.

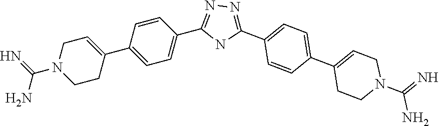 [SMILES: N=C(N)N1CC=C(c2ccc(-c3nnc(-c4ccc(C5=CCN(C(=N)N)CC5)cc4)[nH]3)cc2)CC1]

### Example 56

Synthesis of 4,4-(4,4-(4-Cyclohexyl-4H-1,2,4-triazole-3,5-diyl)bis(4,1-phenylene))bis (5,6-dihydropyridine-1(2H)-carboximidamide): A flask was charged with 820 mg (0.82 mmol) of tert-butyl (4,4-(4,4-(4-cyclohexyl-4H-1,2,4-triazole-3,5-diyl)bis(4,1-phenylene)) bis(5,6-dihydropyridine-4,1(2H)-diyl))bis(methanetriyl)tetra carbamate which was dissolved with a mixture of 1000 mg (9.26 mmol) of anisole and 8 mL of dichloromethane. Then 10 mL (130 mmol) of trifluoroacetic acid added over five minutes with stirring and stirred for 18 hours. LCMS indicted reaction was complete and reaction concentrated under vacuum. Oil was triturated with 10 mL of ether and ether decanted away to yield a solid. Solid was dissolved in N,N-dimethylformamide and was purified by prep HPLC. Yield was 124 mg (20%). ¹H NMR (300 MHz, DMSO-d₆) δ=7.71-7.65 (m, 2H), 7.61-7.44 (m, 6H), 6.40 (br s, 2H), 4.12 (br d, J=3.8 Hz, 1H), 3.92 (s, 2H), 3.76-3.41 (m, 10H), 2.66 (br s, 2H), 1.90 (br t, J=5.8 Hz, 2H), 1.63-1.54 (m, 2H), 1.45 (s, 2H), 1.04 (br d, J=13.9 Hz, 2H). LC/MS method A: R_{f}=4.30 mins., (M+H)⁺=551, purity >90%.

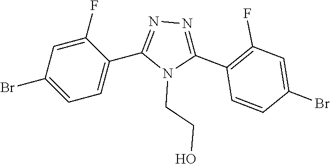 [SMILES: OCCn1c(-c2ccc(Br)cc2F)nnc1-c1ccc(Br)cc1F]

Synthesis of 2-(3,5-bis (4-bromo-2-fluorophenyl)-4H-1,2,4-triazol-4-yl)ethanol: A flask was charged with 1150 mg (2.50 mmol) of (Z)-4-bromo-N-[(1Z)-(4-bromo-2-fluorophenyl)(chloro)methylidene]-2-fluorobenzene-1-carbohydrazonoyl chloride and 15 mL of methanol added to slurry solid. Then ethanolamine (392 mg 6.34 mmol) was added over two minutes. Five minutes later, N,N-diisopropylethylamine (1020 mg, 7.02 mmol) was added over one minute. The reaction stirred for 18 hours at 23° C. Then 4 mL of glacial acetic acid added over five minutes. The reaction was heated at 80° C. for four hours. The reaction was allowed to cool and methanol mostly removed under vacuum and 20 mL of water was added over several minutes. Solids formed and after several hours, solids collected, rinsed with 10 water and ten mL ether. Yield was 680 mg (59%) of product.

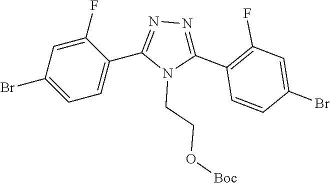 [SMILES: CC(C)(C)OC(=O)OCCn1c(-c2ccc(Br)cc2F)nnc1-c1ccc(Br)cc1F]

Synthesis of 2-(3,5-Bis(4-bromo-2-fluorophenyl)-4H-1,2,4-triazol-4-yl)ethyl tert-butyl carbonate: A flask was charged with 680 mg (1.48 mg) of 2-(3,5-bis(4-bromo-2-fluorophenyl)-4H-1,2,4-triazol-4-yl)ethanol and slurried with 5 mL of 1,4-dioxane. Then 5 mg (0.04 mmol) of 4-dimethylaminopyridine was added, followed by addition of 480 mg (2.20 mmol) was added and reaction heated to 55° C. for 18 hours. LCMS indicated the reaction was complete and reaction was diluted with 5 mL methanol and one hour later the reaction was concentrated under vacuum to yield product in quantitative yield.

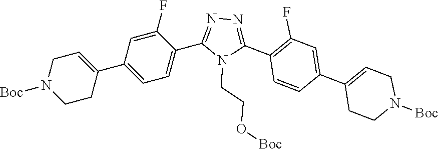 [SMILES: CC(C)(C)OC(=O)OCCn1c(-c2ccc(C3=CCN(C(=O)OC(C)(C)C)CC3)cc2F)nnc1-c1ccc(C2=CCN(C(=O)OC(C)(C)C)CC2)cc1F]

Synthesis of tert-Butyl 4,4′-(4,4′-(4-(2-(tert-butoxycarbonyloxy)ethyl)-4H-1,2,4-triazole-3,5-diyl)bis(3-fluoro-4,1-phenylene))bis(5,6-dihydropyridine-1(2H)-carboxylate): A flask was charged with 1570 mg (2.80 mmol) of 2-(3,5-bis(4-bromo-2-fluorophenyl)-4H-1,2,4-triazol-4-yl)ethyl tert-butyl carbonate, 50 mg (0.22 mmol) of palladium acetate, 230 mg (0.56 mmol) of 2-Dicyclohexylphosphino-2′,6′-dimethoxybiphenyl, 2100 mg (6.72 mmol) of tert-butyl 4-(4,4,5,5-tetramethyl-1,3,2-dioxaborolan-2-yl)-5,6-dihydropyridine-1(2H)-carboxylate, 15 mL of 1,4-dioxane and 10.5 mL of 2.0 M potassium carbonate. The flask was swept with nitrogen for five minutes and heated at 95° C. for 18 hours. The reaction was complete by LCMS and the reaction was allowed to cool and diluted with 10 mL water and 15 mL of ethyl acetate added over two minutes. No solids formed and 20 mL of ethyl acetate and 20 mL water was added. Organic phase separated, washed with 20 mL water, dried and concentrated under vacuum to yield 2.1 grams of tert-butyl 4,4′-(4,4′-(4-(2-hydroxyethyl)-4H-1,2,4-triazole-3,5-diyl)bis(3-fluoro-4,1-phenylene))bis(5,6-dihydropyridine-1(2H)-carboxylate) as an oil in quantitative yield.

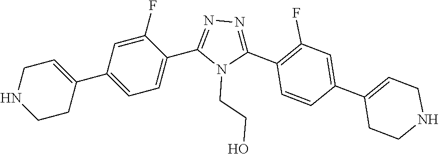 [SMILES: OCCn1c(-c2ccc(C3=CCNCC3)cc2F)nnc1-c1ccc(C2=CCNCC2)cc1F]

Synthesis of 2-(3,5-Bis(2-fluoro-4-(1,2,3,6-tetrahydropyridin-4-yl)phenyl)-4H-1,2,4-triazol-4-yl)ethanol was prepared A flask was charged with 2100 mg (2.8 mmol) of tert-butyl 4,4′-(4,4′-(4-(2-hydroxyethyl)-4H-1,2,4-triazole-3,5-diyl)bis(3-fluoro-4,1-phenylene))bis(5,6-dihydropyridine-1(2H)-carboxylate) was dissolved with 400 mg (3.70 mmol) of anisole and 5 mL dichloromethane. Then 8 mL (10.5 mmol) of trifluoroacetic acid added over five minutes with stirring. After three hours the reaction was complete. The reaction was concentrated under vacuum and triturated with two 25 mL portions of ether and ether decanted away to yield a solid in quantitative yield of 1800 mg of product.

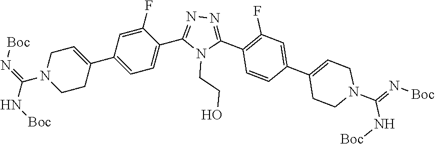 [SMILES: CC(C)(C)OC(=O)/N=C(/NC(=O)OC(C)(C)C)N1CC=C(c2ccc(-c3nnc(-c4ccc(C5=CCN(/C(=N/C(=O)OC(C)(C)C)NC(=O)OC(C)(C)C)CC5)cc4F)n3CCO)c(F)c2)CC1]

Synthesis of tert-Butyl (4,4′-(4,4′-(4-(2-hydroxyethyl)-4H-1,2,4-triazole-3,5-diyl)bis(3-fluoro-4,1-phenylene))bis(5,6-dihydropyridine-4,1(2H)-diyl))bis(methanetriyl) tetracarbamate: A flask was charged with 1800 mg (2.70 mmol) of 2-(3,5-bis(2-fluoro-4-(1,2,3,6-tetrahydropyridin-4-yl)phenyl)-4H-1,2,4-triazol-4-yl)ethanol bis trifluoroacetic acid salt and 10 mL N,N-dimethylformamide and 4 mL dichloromethane. Then 1910 mg (18.9 mmol) of triethylamine added over five minutes. Then 2000 mg (6.48 mmol) of tert-butyl (1H-pyrazol-1-yl)methanediylidenedicarbamate reagent added over several minutes. The reaction was stirred at 23° C. for two hours. Then 800 mg (2.58 mmol) of tert-butyl (1H-pyrazol-1-yl)methanediylidenedicarbamate reagent was added at once. After stirring for 18 hours at room temp the reaction was diluted with 40 mL ethyl acetate and then washed with three 20 mL potions of water. The organic phase was dried with sodium sulfate and concentrated under vacuum, purified by normal phase chromatography with column eluted with (20% MeOH/dichloromethane)/dichloromethane step gradient. Yield was 1120 mg of product as an oil. (44% yield).

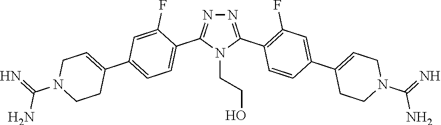 [SMILES: N=C(N)N1CC=C(c2ccc(-c3nnc(-c4ccc(C5=CCN(C(=N)N)CC5)cc4F)n3CCO)c(F)c2)CC1]

### Example 57

Synthesis of 4,4′-(4,4′-(4-(2-Hydroxyethyl)-4H-1,2,4-triazole-3,5-diyl)bis(3-fluoro-4,1-phenylene))bis(5,6-dihydropyridine-1(2H)-carboximidamide): A flask was charged with 1120 mg (1.18 mmol) of tert-butyl (4,4′-(4,4′-(4-(2-hydroxyethyl)-4H-1,2,4-triazole-3,5-diyl)bis(3-fluoro-4,1-phenylene))bis(5,6-dihydropyridine-4,1(2H)-diyl))bis(methane-1-yl-1,1-diylidene)tetracarbamate was slurried with 500 mg (4.63 mmol) of anisole and 6 mL of dichloromethane. Then 10 mL (13.1 mmol) of trifluoroacetic acid was added over five minutes with stirring. Two hours later reaction was complete and was concentrated under vacuum. Oil was triturated with two 30 mL portions of ether and ether decanted away to yield a solid. The solid was dissolved with 50 mL of N,N-dimethylformamide. The material was purified by prep HPLC on a Waters C-18 Sunfire column with a 20 minute method starting with 1% acetonitrile to 25% using a 40 mL, per minute flow rate. After freeze drying yield was 140 mg (31%). ¹H NMR (300 MHz, DMSO-d₆) δ=7.70-7.48 (m, 14H), 6.49-6.45 (m, 2H), 4.12 (br d, J=3.6 Hz, 4H), 3.91 (br t, J==5.5 Hz, 2H), 3.66-3.60 (m, 4H), 3.33-3.27 (m, 2H), 3.19-3.13 (m, 2H). LC/MS method A: R_{f}=2.69 mins., (M+H)⁺=549, purity >95%.

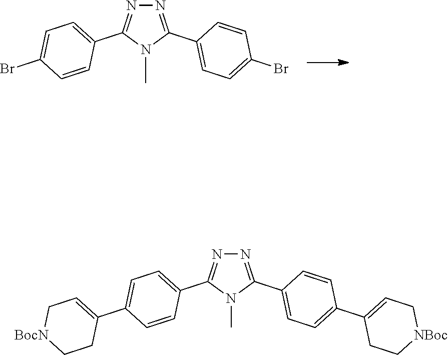 [SMILES: Cn1c(-c2ccc(Br)cc2)nnc1-c1ccc(Br)cc1 ; Cn1c(-c2ccc(C3=CCN(C(=O)OC(C)(C)C)CC3)cc2)nnc1-c1ccc(C2=CCN(C(=O)OC(C)(C)C)CC2)cc1]

Synthesis of tert-Butyl 4-{4-[5-(4-{1-[(tert-butoxy)carbonyl]-1,2,3,6-tetrahydropyridin-4-yl}phenyl)-4-methyl-4H-1,2,4-triazol-3-yl]phenyl}-1,2,3,6-tetrahydropyridine-1-carboxylate: A mixture of 3,5-bis(4-bromophenyl)-4-methyl-4H-1,2,4-triazole (50 mg, 0.13 mmol), tert-butyl 4-(4,4,5,5-tetramethyl-1,3,2-dioxaborolan-2-yl)-1,2,3,6-tetrahydropyridine-1-carboxylate (94 mg, 0.30 mmol), Dicyclohexyl(2′,6′-dimethoxy biphenyl-2-yl) phosphine (8 mg, 20 μmol), Palladium acetate (2.0 mg, 8 μmol), potassium carbonate (108 mg, 0.78 mmol), water (0.4 mL) and 1,4-dioxane (1 mL) was purged with nitrogen, stirred and heated to 90° C. for 20 hours. The mixture was diluted with dichloromethane (20 mL) and water (20 mL) and the aqueous layer was extracted with dichloromethane (20 mL). The combined dichloromethane layers were dried (Na₂SO₄) and evaporated. The crude product mixture was chromatographed on silica gel with a gradient of 50% dichloromethane and 50% ethyl acetate to 100% ethyl acetate to leave a beige solid (53 mg, 68%). ¹H-NMR (CDCl₃) δ 7.73 (d, 4H, J=8.5 Hz), 7.54 (d, 4H, J=8.5 Hz), 6.17 (bm, 2H), 4.12 (m, 4H), 3.74 (s, 3H), 3.68 (m, 4H), 2.58 (m, 4H), 1.51 (s, 18H). LCMS method A R_{f}=5.70 mins, purity >95%, (M+H)⁺=599.

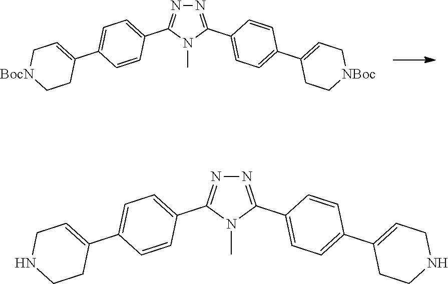 [SMILES: Cn1c(-c2ccc(C3=CCN(C(=O)OC(C)(C)C)CC3)cc2)nnc1-c1ccc(C2=CCN(C(=O)OC(C)(C)C)CC2)cc1 ; Cn1c(-c2ccc(C3=CCNCC3)cc2)nnc1-c1ccc(C2=CCNCC2)cc1]

Synthesis of 4-(4-{4-Methyl-5-[4-(1,2,3,6-tetrahydropyridin-4-yl)phenyl]-4H-1,2,4-triazol-3-yl}phenyl)-1,2,3,6-tetrahydropyridine: A solution of tert-butyl 4-{4-[5-(4-{1-[(tert-butoxy)carbonyl]-1,2,3,6-tetrahydropyridin-4-yl}phenyl)-4-methyl-4H-1,2,4-triazol-3-yl]phenyl}-1,2,3,6-tetrahydropyridine-1-carboxylate (28 mg, 48 μmol) in trifluoroacetic acid (1.5 mL) and dichloromethane (1.5 mL) was stirred for 5 hours then evaporated to leave the product as a crystalline solid (46 mg, 100%).

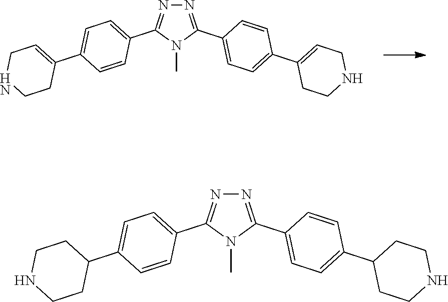 [SMILES: Cn1c(-c2ccc(C3=CCNCC3)cc2)nnc1-c1ccc(C2=CCNCC2)cc1 ; Cn1c(-c2ccc(C3CCNCC3)cc2)nnc1-c1ccc(C2CCNCC2)cc1]

Synthesis of 4-(4-{4-Methyl-5-[4-(piperidin-4-yl)phenyl]-4H-1,2,4-triazol-3-yl}phenyl)piperidine: A solution of 4-(4-{4-methyl-5-[4-(1,2,3,6-tetrahydropyridin-4-yl) phenyl]-4H-1,2,4-triazol-3-yl}phenyl)-1,2,3,6-tetrahydropyridine (26 mg, (38 μmol) in methanol (3 mL) was hydrogenated over 10% palladium on carbon (10 mg) at 45 psi initial hydrogen pressure for 24 hours. The catalyst was filtered and the solvent was evaporated to leave a clear thick gum (28 mg, 100%). ¹H-NMR (CD₃OD) δ 7.77 (d, 4H, J=8.5 Hz), 7.54 (d, 4H, J=8.5 Hz), 3.75 (s, 3H), 3.47-3.60 (m, 4H), 3.00-3.25 (m, 7H), 1.90-2.10 (m, 7H). LCMS method A R_{f}=2.14 mins, purity >95%, (M+H)⁺=402.

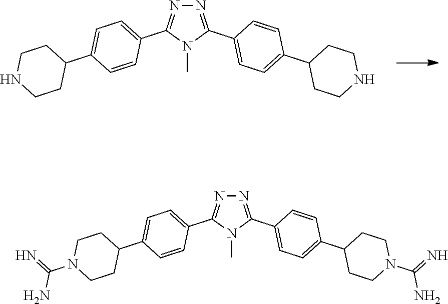 [SMILES: Cn1c(-c2ccc(C3CCNCC3)cc2)nnc1-c1ccc(C2CCNCC2)cc1 ; Cn1c(-c2ccc(C3CCN(C(=N)N)CC3)cc2)nnc1-c1ccc(C2CCN(C(=N)N)CC2)cc1]

Synthesis of 4-(4-{5-[4-(1-Carbamimidoylpiperidin-4-yl)phenyl]-4-methyl-4H-1,2,4-triazol-3-yl}phenyl)piperidine-1-carboximidamide: A solution of 4-(4-{4-methyl-5-[4-(piperidin-4-yl)phenyl]-4H-1,2,4-triazol-3-yl}phenyl)piperidine (23 mg, 31 μmol), N—((N′,N″-bis-tert-butyloxycarbonyl)amidino)pyrrazole (29 mg, 93 μmol) and N,N-diisopropylethylamine (32 mg, 248 μmol, 44 μl) in methanol (0.5 mL) was stirred for 24 hours and evaporated to dryness. The crude product was dissolved in dichloromethane (1 mL) and trifluoroacetic acid (1 mL) and stirred for 3 hours. The solvents were evaporated and the crude product was purified by preparative reverse phase HPLC and the product fractions were combined and lyophilized to leave a white powder (12 mg, 47%). ¹H-NMR (CD₃OD) δ 7.50 (d, 4H, J=8.2 Hz), 7.54 (d, 4H, J=8.2 Hz), 4.05 (m, 4H), 3.75 (s, 3H), 3.20-3.40 (m, 4H), 2.98-3.10 (m, 2H), 1.97-2.05 (m, 4H), 1.75-1.90 (m, 4H).

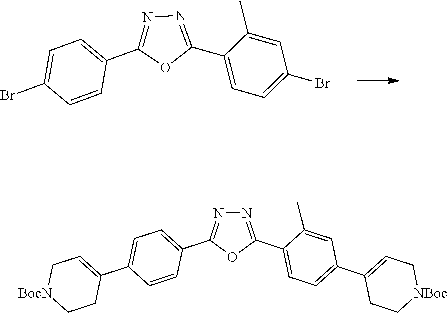 [SMILES: Cc1cc(Br)ccc1-c1nnc(-c2ccc(Br)cc2)o1 ; Cc1cc(C2=CCN(C(=O)OC(C)(C)C)CC2)ccc1-c1nnc(-c2ccc(C3=CCN(C(=O)OC(C)(C)C)CC3)cc2)o1]

Synthesis of tert-Butyl 4-{4-[5-(4-{1-[(tert-Butoxy)carbonyl]-1,2,3,6-tetrahydropyridin-4-yl}phenyl)-1,3,4-oxadiazol-2-yl]-3-methylphenyl}-1,2,3,6-tetrahydropyridine-1-carboxylate: A mixture of 2-(4-bromo-2-methyl-phenyl)-5-(4-bromo-phenyl)-[1,3,4]oxadiazole (0.60 g, 1.52 mmol), tert-butyl-4-(4,4,5,5-tetramethyl-1,3,2-dioxaborolan-2-yl)-1,2,3,6-tetrahydro pyridine-1-carboxylate (1.1 g, 3.5 mmol), palladium tetrakis(triphenylphosphine) (185 mg, 0.16 mmol), 2.0 M potassium bicarbonate (4.4 mL, 8.8 mmol) and 1,4-dioxane (1 mL) was purged with nitrogen, stirred and heated to 80° C. for 20 hours. The mixture was cooled to 20° C., diluted with water (20 mL) stirred for 15 minutes, then filtered, washed with water and dried under vacuum. Dissolved in minimal dichloromethane and chromatographed on silica gel with a gradient of ethyl acetate in dichloromethane from 10% to 20% to 30%. Left a light yellow solid (0.78 g, 83%). ¹H-NMR (CDCl₃) δ 8.10 (d, 4H, J=8.5 Hz), 7.54 (d, 4H, J=8.5 Hz), 6.20 (m, 2H), 4.13 (m, 4H), 3.66 (m, 4H), 2.58 (m, 4H), 1.51 (s, 18H). LCMS method A R_{f}=7.28 mins, purity >95%, (M+H)⁺=585.

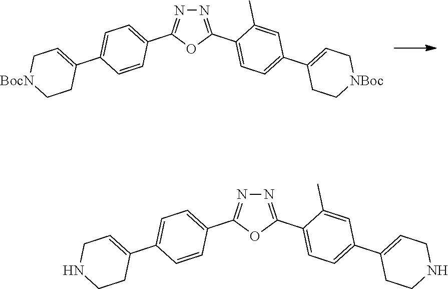 [SMILES: Cc1cc(C2=CCN(C(=O)OC(C)(C)C)CC2)ccc1-c1nnc(-c2ccc(C3=CCN(C(=O)OC(C)(C)C)CC3)cc2)o1 ; Cc1cc(C2=CCNCC2)ccc1-c1nnc(-c2ccc(C3=CCNCC3)cc2)o1]

Synthesis of 4-(3-Methyl-4-{5-[4-(1,2,3,6-tetrahydropyridin-4-yl)phenyl]-1,3,4-oxadiazol-2-yl}phenyl)-1,2,3,6-tetrahydropyridine: A solution of tert-butyl 4-{4-[5-(4-{1-[(tert-butoxy)carbonyl]-1,2,3,6-tetrahydropyridin-4-yl}phenyl)-1,3,4-oxadiazol-2-yl]-3-methylphenyl}-1,2,3,6-tetrahydropyridine-1-carboxylate (0.67 g, 1.12 mmol) in dichloromethane (5 mL) and trifluoroacetic acid (5 mL) was stirred for 2 hours then evaporated to dryness to leave a tan solid (0.67 g, 100%).

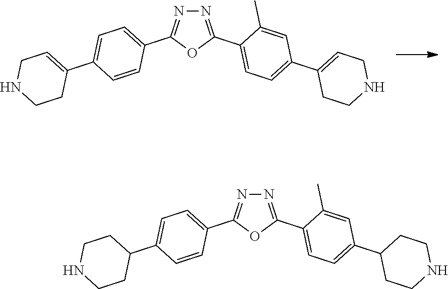 [SMILES: Cc1cc(C2=CCNCC2)ccc1-c1nnc(-c2ccc(C3=CCNCC3)cc2)o1 ; Cc1cc(C2CCNCC2)ccc1-c1nnc(-c2ccc(C3CCNCC3)cc2)o1]

Synthesis of 4-(3-Methyl-4-{5-[4-(piperidin-4-yl)phenyl]-1,3,4-oxadiazol-2-yl}phenyl)piperidine: A solution of 4-(3-methyl-4-{5-[4-(1,2,3,6-tetrahydropyridin-4-yl)phenyl]-1,3,4-oxadiazol-2-yl}phenyl)-1,2,3,6-tetrahydropyridine (0.10 g, 0.16 mmol) was hydrogenated in methanol (3 mL) over 5% palladium on carbon (20 mg) at 45 psi initial hydrogen pressure for 18 hours. The catalyst was filtered and the solvents evaporated to leave the product as a crystalline solid (100 mg, 100%). ¹H-NMR (CD₃OD) δ 8.13 (d, 4H, J=8.2 Hz), 7.54 (d, 4H, J=8.2 Hz), 3.57 (m, 4H), 3.18 (td, 4H, J=122.9 Hz, J=2.9 Hz), 3.05 (tt, 2H, J=12.0 Hz, J=3.5 Hz), 2.15 (m, 4H), 1.95 (m, 4H). LCMS method A R_{f}=2.74 mins, purity >95%, (M+H)⁺=389.

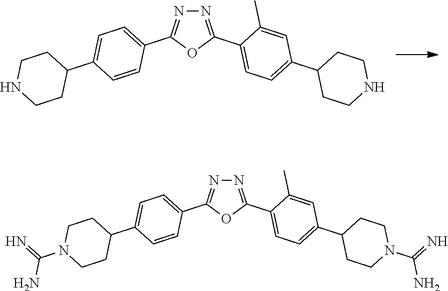 [SMILES: Cc1cc(C2CCNCC2)ccc1-c1nnc(-c2ccc(C3CCNCC3)cc2)o1 ; Cc1cc(C2CCN(C(=N)N)CC2)ccc1-c1nnc(-c2ccc(C3CCN(C(=N)N)CC3)cc2)o1]

### Example 58

Synthesis of 4-(4-{5-[4-(1-Carbamimidoylpiperidin-4-yl)phenyl]-1,3,4-oxadiazol-2-yl}-3-methylphenyl)piperidine-1-carboximidamide: A solution of 4-(3-methyl-4-{5-[4-(piperidin-4-yl)phenyl]-1,3,4-oxadiazol-2-yl}phenyl)piperidine (30 mg, 49 mmol), N—((N′,N″-bis-tert-butyloxycarbonyl)amidino)pyrrazole (45 mg, 0.15 mmol) and N,N-diisopropylethylamine (40 mg, 0.39 mmol, 54 μl) in methanol (0.5 mL) was stirred for 18 hours. The product was purified by preparative reverse phase HPLC and the product fractions were evaporated in vacuo. The crude product was dissolved in trifluoroacetic acid (1 mL) and dichloromethane (1 mL) and stirred for 2 hours then evaporated to dryness. The product was dissolved in water (3 mL) and acetonitrile (1 mL) and lyophilized to leave a beige powder (23 mg, 67%). ¹H-NMR (CD₃OD) δ 8.10 (d, 4H, J=8.5 Hz), 7.53 (d, 4H, J=8.5 Hz), 4.03 (m, 4H), 3.22 (m, 6H), 3.02 (m, 2H), 2.01 (m, 4H), 1.80 (m, 4H). LCMS method A R_{f}=3.20 mins, purity >95%, (M+H)⁺=473.

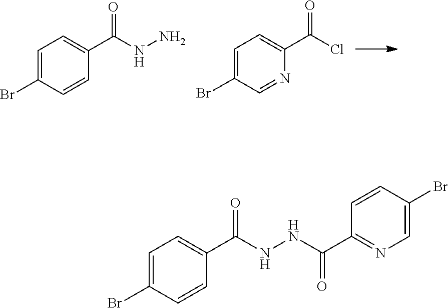 [SMILES: NNC(=O)c1ccc(Br)cc1 ; O=C(Cl)c1ccc(Br)cn1 ; O=C(NNC(=O)c1ccc(Br)cn1)c1ccc(Br)cc1]

Synthesis of 5-bromo-N′-(4-bromobenzoyl)pyridine-2-carbohydrazide: A solution of 4-bromophenylhydrazide (1.0 g, 4.7 mmol) and N,N-(diisopropyl)ethylamine (0.60 g, 4.7 mmol, 0.83 mL) in N,N-dimethylformamide (20 mL) was cooled in an ice bath and treated with 5-bromopyridin-2-yl acid chloride (1.2 g, 5.2 mmol). The mixture stirred and warmed to 20° C. over 2 hours then stirred for 18 hours. The reaction mixture was diluted with water (30 mL), stirred 30 minutes then filtered, washed with water and ether and dried under vacuum to leave 1.8 g (96%) of tan solid. ¹H-NMR (DMSO-d₆) δ 10.74 (bs, 1H), 10.65 (bs, 1H), 8.84 (s, 1H), 8.30 (d, 1H, J=8.2 Hz), 7.90 (d, 1H, J=8.2 Hz), 7.84 (d, 2H, J=7.8 Hz), 7.73 (d, 2H, J=7.8 Hz). LCMS method A R_{f}=4.18 mins, purity >95%, (M+H)⁺=400.

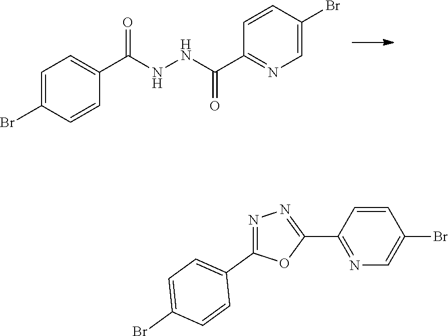 [SMILES: O=C(NNC(=O)c1ccc(Br)cn1)c1ccc(Br)cc1 ; Brc1ccc(-c2nnc(-c3ccc(Br)cn3)o2)cc1]

Synthesis of bis(4-Bromophenyl)-1,3,4-oxadiazole: A solution of 5-bromo-N′-(4-bromobenzoyl)pyridine-2-carbohydrazide (0.50 g, 1.20 mmol) phosphorous oxychloride (5 mL) was heated to reflux for 18 hours. The reaction mixture was cooled in ice and treated with ice (20 g). After 30 minutes the reaction mixture was poured into 1M sodium carbonate (125 mL), stirred for 30 minutes then filtered, washed with water and dried under vacuum. The product was purified by preparative reverse phase HPLC to leave 135 mg (30%) of light tan solid. ¹H-NMR (CDCl₃) δ 8.80 (bs, 1H), 8.14 (d, 1H, J=8.2 Hz), 8.00 (d, 2H, J=8.8 Hz), 7.98 (dd, 1H, J=8.2 Hz, J=1.8 Hz), 7.63 (d, 2H, J=8.8 Hz). LCMS method A R_{f}=5.55 mins, purity >95%, (M+H)⁺=382.

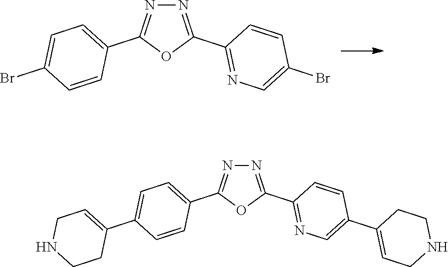 [SMILES: Brc1ccc(-c2nnc(-c3ccc(Br)cn3)o2)cc1 ; C1=C(c2ccc(-c3nnc(-c4ccc(C5=CCNCC5)cn4)o3)cc2)CCNC1]

Synthesis of 6-{5-[4-(1,2,3,6-Tetrahydropyridin-4-yl)phenyl]-1,3,4-oxadiazol-2-yl}-1′,2′,3′,6′-tetrahydro-3,4′-bipyridine: A mixture of bis(4-bromophenyl)-1,3,4-oxadiazole 50 mg, 0.13 mmol), tert-butyl 4-(4,4,5,5-tetramethyl-1,3,2-dioxaborolan-2-yl)-1,2,3,6-tetrahydropyridine-1-carboxylate (122 mg, 0.39 mmol), dicyclohexyl(2′,6′-dimethoxybiphenyl-2-yl) phosphine (3.7 mg, 9 μmol), palladium acetate (2.0 mg, 8 μmol), potassium carbonate (145 mg, 1.0 mmol), water (0.4 mL) and 1,4-dioxane (1 mL) was purged with nitrogen, stirred and heated to 90° C. for 20 hours. The mixture was diluted with dichloromethane (20 mL) and water (20 mL) and the aqueous layer was extracted with dichloromethane (20 mL). The combined dichloromethane layers were dried (Na₂SO₄) and evaporated. The crude product mixture was dissolved in trifluoroacetic acid (1 mL) and dichloromethane (1 mL) and stirred for 2 hours. The mixture was evaporated, purified by preparative reverse phase HPLC and the product fractions were lyophilized to leave the product as a white solid (tris trifluoroacetic acid salt, 64 mg, 58%). ¹H-NMR (CD₃OD) δ 8.91 (d, 1H, J=1.5 Hz), 8.33 (d, 1H, J=8.5 Hz), 8.22 (d, 2H, J=8.8 Hz), 8.17 (dd, 1H, J=8.2 Hz, J=2.0 Hz), 7.75 (d, 2H, J=8.8 Hz), 6.48 (m, 0.1H), 6.38 (m, 1H), 3.94 (m, 4H), 3.53 (m, 4H), 2.88 (m, 4H). LCMS method A R_{f}=2.39 mins, purity >95%, (M+H)⁺=386.

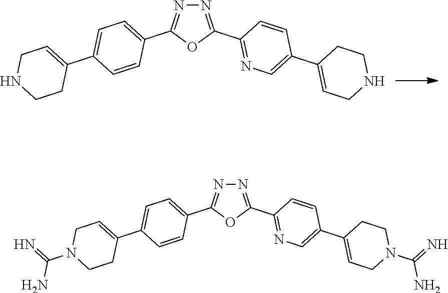 [SMILES: C1=C(c2ccc(-c3nnc(-c4ccc(C5=CCNCC5)cn4)o3)cc2)CCNC1 ; N=C(N)N1CC=C(c2ccc(-c3nnc(-c4ccc(C5=CCN(C(=N)N)CC5)cn4)o3)cc2)CC1]

### Example 59

Synthesis of 6-{5-[4-(1-Carbamimidoyl-1,2,3,6-tetrahydropyridin-4-yl)phenyl]-1,3,4-oxadiazol-2-yl}-1′,2′,3′,6′-tetrahydro-[3,4′-bipyridine]-1′-carboximidamide: A solution of 6-{5-[4-(1,2,3,6-tetrahydropyridin-4-yl)phenyl]-1,3,4-oxadiazol-2-yl}-1′,2′,3′,6′-tetrahydro-3,4′-bipyridine (60 mg, 71 Imo), N—((N′,N″-bis-tert-butyloxycarbonyl)amid no)pyrrazole (66 mg, 0.21 mmol) and N,N-diisopropylethylamine (92 mg, 0.71 mmol, 127 μl) in methanol (2 mL) was stirred for 18 hours. N,N-dimethylformamide (1 mL) was added to solubilize a precipitate and the product was purified by reverse phase HPLC. The product fractions were combined, treated with saturated sodium bicarbonate solution, extracted with dichloromethane (2×50 mL), dried and evaporated to leave product (58 mg, 94%) as a gum. The product was dissolved in 1,4-dioxane (2 mL) and 4N HCl in 1,4-dioxane (2 mL). After stirring for 20 h the solvents were evaporated and the product was lyophilized to leave a beige solid (51 mg, 94%). ¹H-NMR (CD₃OD) δ 8.95 (d, 1H, J=2.3 Hz), 8.26 (d, 1H, J=7.6 Hz), 8.10 (d, 2H, J=8.5 Hz), 7.74 (d, 2H, J=8.8 Hz), 7.45 (bs, 8H), 6.55 (m, 1H), 6.42 (m, 1H), 4.15 (m, 4H), 3.65 (m, 4H), 2.65 (m, 4H). LCMS method A R_{f}=2.90 mins, purity >95%, (M+H)⁺=470.

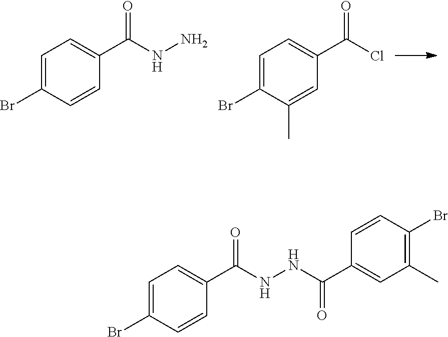 [SMILES: NNC(=O)c1ccc(Br)cc1 ; Cc1cc(C(=O)Cl)ccc1Br ; Cc1cc(C(=O)NNC(=O)c2ccc(Br)cc2)ccc1Br]

Synthesis of 4-Bromo-N′-(4-bromobenzoyl)-3-methylbenzohydrazide: A solution of 4-bromophenylhydrazide (1.0 g, 4.7 mmol) and N,N-(diisopropyl)ethylamine (0.60 g, 4.7 mmol, 0.83 mL) in N,N-dimethylformamide (20 mL) was cooled in an ice bath and treated with 4-bromo-3methylbenzoic acid chloride (1.2 g, 5.2 mmol). The mixture stirred and warmed to 20° C. over 2 hours then stirred 18 hours The reaction mixture was diluted with water (30 mL), stirred 30 minutes then filtered, washed with water and ether and dried under vacuum to leave 1.5 g (77%) of tan solid.

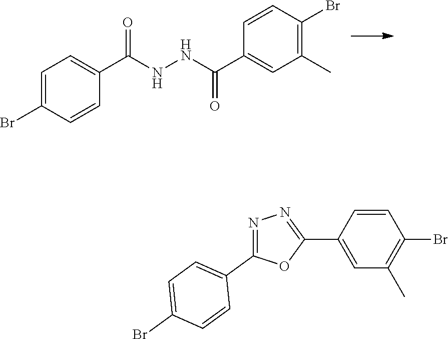 [SMILES: Cc1cc(C(=O)NNC(=O)c2ccc(Br)cc2)ccc1Br ; Cc1cc(-c2nnc(-c3ccc(Br)cc3)o2)ccc1Br]

Synthesis of 2-(4-Bromo-3-methylphenyl)-5-(4-bromophenyl)-1,3,4-oxadiazole: A solution of 4-bromo-N′-(4-bromobenzoyl)-3-methylbenzohydrazide (0.82 g, 2.0 mmol), phosphorous oxychloride (3 mL) and acetonitrile (15 mL) was heated to reflux for 18 hours. The reaction mixture was cooled in ice and treated with ice (5 g). After 30 minutes the mixture was treated with more water (10 mL) then filtered, washed with water and dried under vacuum to leave 0.69 g (88%) of light tan solid. ¹H-NMR (DMSO-d₆) δ 8.10 (dd, 1H, J=8.5 Hz. J=1.5 Hz), 8.06 (d, 2H, J=8.5 Hz), 7.81 (d, 2H, J=8.5 Hz), 7.80 (m, 2H), 2.45 (s, 3H). LCMS method A R_{f}=6.62 mins, purity >95%, (M+H)⁺=395.

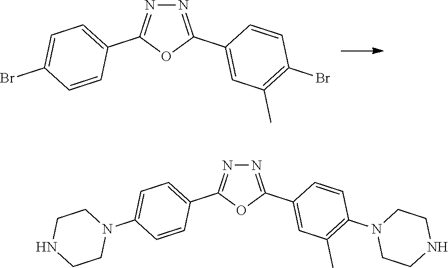 [SMILES: Cc1cc(-c2nnc(-c3ccc(Br)cc3)o2)ccc1Br ; Cc1cc(-c2nnc(-c3ccc(N4CCNCC4)cc3)o2)ccc1N1CCNCC1]

Synthesis of 1-(4-{5-[3-Methyl-4-(piperazin-1-yl)phenyl]-1,3,4-oxadiazol-2-yl}phenyl)piperazine: A mixture of 2-(4-bromo-3-methylphenyl)-5-(4-bromophenyl)-1,3,4-oxadiazole (50 mg, 0.13 mmol), N-(tert-butyloxcarbonyl)piperazine (71 mg, 0.38 mmol), dicyclohexyl(2′,6′-dimethoxybiphenyl-2-yl) phosphine (4.1 mg, 10 moll), bispalladium-tri(1,3-dibenzylidene) acetone (3 mg (3.3 μmol), sodium tert-butoxide (37 mg, 0.39 mmol) and tolune (1 mL) was purged with nitrogen, stirred and heated top 100° C. for 18 hours. The reaction mixture was cooled to 20° C., diluted with water (25 mL) and extracted with dichloromethane (2×25 mL). The combined extracts were dried (MgSO₄) and evaporated to leave a solid which was recrystallized from methanol to leave a light yellow solid (48 mg, 61%). The solid was dissolved in trifluoroacetic acid (1 mL) and dichloromethane (1 mL) and stood for 2 hours. The solvents were evaporated and the product was lyophilized to leave a tan solid (52 mg, 54%). ¹H-NMR (CD₃OD) δ 8.04 (d, 2H, J=9.1 Hz), 7.99 (m, 2H), 7.28 (d, 1H, J=8.2 Hz), 7.20 (d, 2H, J=9.2 Hz), 3.32 (m, 4H), 3.41 (m, 8H), 3.25 (m, 4H), 2.44 (s, 3H). LCMS method A R_{f}=2.58 mins, purity >95%, (M+H)⁺=405.

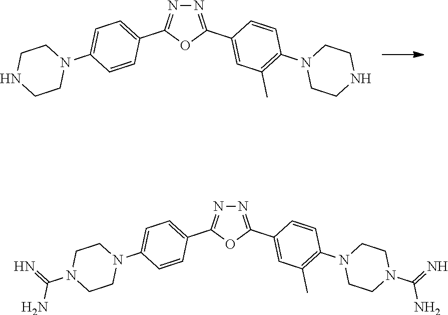 [SMILES: Cc1cc(-c2nnc(-c3ccc(N4CCNCC4)cc3)o2)ccc1N1CCNCC1 ; Cc1cc(-c2nnc(-c3ccc(N4CCN(C(=N)N)CC4)cc3)o2)ccc1N1CCN(C(=N)N)CC1]

### Example 60

Synthesis of 4-(4-{5-[4-(4-Carbamimidoylpiperazin-1-yl)-3-methylphenyl]-1,3,4-oxadiazol-2-yl}phenyl)piperazine-1-carboximidamide: A solution of 1-(4-{5-[3-methyl-4-(piperazin-1-yl)phenyl]-1,3,4-oxadiazol-2-yl}phenyl)piperazine (45 mg, 60 μmol), N—((N′,N″-bis-tert-butyloxycarbonyl)amidino)pyrrazole (56 mg, 0.18 mmol) and N,N-diisopropyl ethylamine (77 mg, 0.60 mmol, 108 μl) in methanol (2 mL) was stirred for 18 h. The product was purified by reverse phase HPLC. The product fractions were combined, treated with saturated sodium bicarbonate solution, extracted with dichloromethane (2×50 mL), dried and evaporated to leave product (49 mg, 92%) as a gum. The product was dissolved in trifluoroacetic acid (1 mL) and dichloromethane (I mL). After stirring for 18 hours the solvents were evaporated, the crude product was purified by preparative reverse phase HPLC and the product fractions were lyophilized to leave a beige solid (20 mg, 40%). ¹H-NMR (CD₃OD) δ 8.01 (d, 2H, J=9.2 Hz), 7.96 (d, 1H, J=2.4 Hz), 7.92 (dd, 1H, J=8.5 Hz, J=2.4 Hz), 7.25 (d, 1H, J=8.5 Hz), 7.13 (d, 2H, J=9.2 Hz), 3.68 (m, 8H), 3.55 (m, 4H), 3.10 (m, 4H), 2.44 (s, 3H). LCMS method A R_{f}=2.90 mins, purity >95%, (M+H)⁺=489.

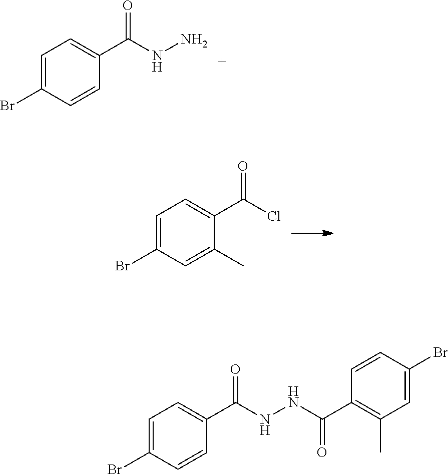

Synthesis of 4-Bromo-N′-(4-bromobenzoyl)-2-methylbenzohydrazide: A solution of 4-bromophenylhydrazide (1.0 g, 4.7 mmol) and N,N-diisopropylethylamine (0.60 g, 4.7 mmol, 0.83 mL) in N,N-dimethylformamide (20 mL) was cooled in an ice bath and treated with 4-bromo-2-methylbenzoic acid chloride (1.2 g, 5.2 mmol). The mixture stirred and warmed to 20° C. over 2 hours then stirred 18 hours. The reaction mixture was diluted with water (30 mL), stirred 30 minutes then filtered, washed with water and ether and dried under vacuum to leave 1.7 g (88%) of tan solid. ¹H-NMR (DMSO-d₆) δ 10.6 (bs, 1H), 10.32 (bs, 1H), 7.85 (d, 2H, J=8.5 Hz), 7.73 (d, 2H, J=8.5 Hz), 7.55 (d, 1H, J=1.8 Hz), 7.50 (dd, 1H, J=8.2 Hz, J=1.8 Hz), 7.36 (d, 1H, J=8.2 Hz), 2.41 (s, 3H). LCMS method A R_{f}=4.53 mins, purity >95%, (M+H)⁺=411.

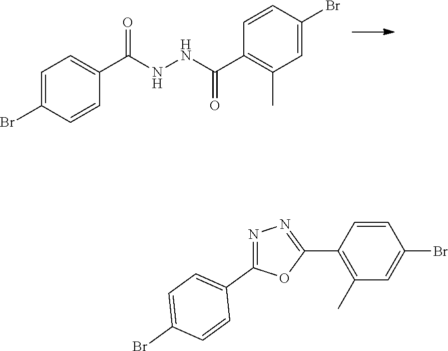 [SMILES: Cc1cc(Br)ccc1C(=O)NNC(=O)c1ccc(Br)cc1 ; Cc1cc(Br)ccc1-c1nnc(-c2ccc(Br)cc2)o1]

Synthesis of 2-(4-Bromo-2-methylphenyl)-5-(4-bromophenyl)-1,3,4-oxadiazole: A solution of 4-bromo-N′-(4-bromobenzoyl)-2-methylbenzohydrazide (0.82 g, 2.0 mmol), phosphorous oxychloride (3 mL) and acetonitrile (15 mL) was heated to reflux for 18 hours. The reaction mixture was cooled in ice and treated with ice (5 g). After 30 minutes the mixture was treated with more water (10 mL) then filtered, washed with water and dried under vacuum to leave 0.79 g (100%) of light tan solid. ¹H-NMR (DMSO-d₆) δ 8.04 (d, 2H, J=8.5 Hz), 8.01 (d, 1H, J=8.2 Hz), 7.84 (d, 2H, J=8.5 Hz), 7.74 (d, 1H, J=1.7 Hz), 7.64 (dd, 1H, J=8.5 Hz, J=1.7 Hz), 2.68 (s, 3H). LCMS method A R^{f}=6.63 mins, purity >95%, (M+H)⁺=395.

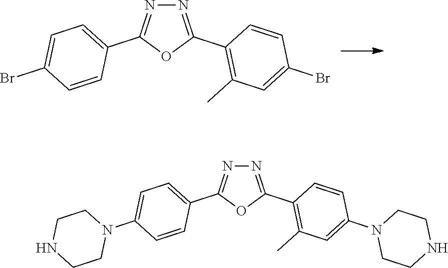 [SMILES: Cc1cc(Br)ccc1-c1nnc(-c2ccc(Br)cc2)o1 ; Cc1cc(N2CCNCC2)ccc1-c1nnc(-c2ccc(N3CCNCC3)cc2)o1]

Synthesis of 1-(4-{5-[2-Methyl-4-(piperazin-1-yl)phenyl]-1,3,4-oxadiazol-2-yl}phenyl)piperazine: A mixture of 2-(4-bromo-2-methylphenyl)-5-(4-bromophenyl)-1,3,4-oxadiazole (50 mg, 0.13 mmol), N-(tert-butyloxcarbonyl)piperazine (71 mg, 0.38 mmol), dicyclohexyl(2′,6′-dimethoxybiphenyl-2-yl) phosphine (4.1 mg, 0.10 μmol), bispalladium-tri(1,3-dibenzylidene) acetone (3 mg (3.3 mop, sodium tert-butoxide (37 mg, 0.39 mmol) and tolune (1 mL) was purged with nitrogen, stirred and heated top 100° C. for 18 hours. The reaction mixture was cooled to 20° C., diluted with water (25 mL) and extracted with dichloromethane (2×25 mL). The combined extracts were dried (MgSO₄) and evaporated to leave a solid which was purified by silica chromatography eluted with a gradient of ethyl acetate in hexanes (10%/25%/50%) to leave a light yellow solid (42 mg, 53%). The solid was dissolved in trifluoroacetic acid (1 mL) and dichloromethane (1 mL) and stood for 2 hours. The solvents were evaporated and the product was lyophilized to leave a tan solid (51 mg, 53%). ¹H-NMR (CD₃OD) δ 8.04 (d, 2H, J=9.2 Hz), 7.98 (s, 1H), 7.97 (d, 1H. J=8.3 Hz), 7.28 (d, 1H, J=8.3 Hz), 7.20 (d, 2H, J=9.2 Hz), 3.62 (m, 4H), 3.40 (m, 8H, 3.25 (m, 4H), 2.45 (s, 3H). LCMS method A R_{f}=2.58 mins, purity >95%, (M+H)⁺=405.

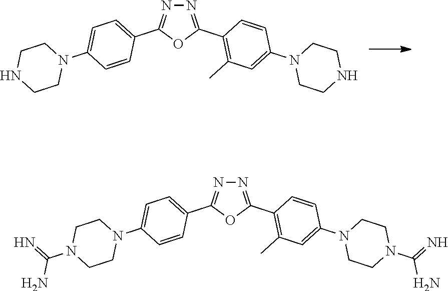 [SMILES: Cc1cc(N2CCNCC2)ccc1-c1nnc(-c2ccc(N3CCNCC3)cc2)o1 ; Cc1cc(N2CCN(C(=N)N)CC2)ccc1-c1nnc(-c2ccc(N3CCN(C(=N)N)CC3)cc2)o1]

### Example 61

Synthesis of 4-(4-{5-[4-(4-Carbamimidoylpiperazin-1-yl)-2-methylphenyl]-1,3,4-oxadiazol-2-yl}phenyl)piperazine-1-carboximidamide: A solution of 1-(4-{5-[2-methyl-4-(piperazin-1-yl)phenyl]-1,3,4-oxadiazol-2-yl}phenyl)piperazine (45 mg, 60 μmol), N—((N′,N″-bis-tert-butyloxycarbonyl)amidino)pyrrazole (56 mg, 0.18 mmol) and N,N-(diisopropyl)ethylamine (77 mg, 0.60 mmol, 108 μl) in methanol (2 mL) was stirred for 18 h. The product was purified by reverse phase HPLC. The product fractions were combined, treated with saturated sodium bicarbonate solution, extracted with methylene chloride (2×50 mL), dried and evaporated to leave product (49 mg, 92%) as a gum. The product was dissolved in trifluoroacetic acid (1 mL) and methylene chloride (1 mL). After stirring for 18 hours the solvents were evaporated, the crude product was purified by preparative reverse phase HPLC and the product fractions were lyophilized to leave a beige solid (27 mg, 54%). ¹H-NMR (CD₃OD) δ 7.99 (d, 2H, J=9.1 Hz), 7.94 (d, 1H, J=9.7 Hz), 7.13 (d, 2H, J=9.1 Hz, J=2.4 Hz), 6.95 (m, 2H), 3.68 (m, 8H), 3.52 (m, 8H), 2.69 (s, 3H). LCMS method A R_{f}=2.87 mins, purity >95%, (M+H)⁺=489.

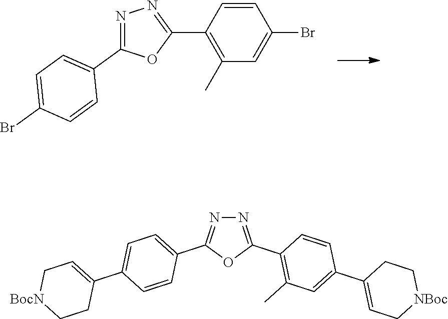 [SMILES: Cc1cc(Br)ccc1-c1nnc(-c2ccc(Br)cc2)o1 ; Cc1cc(C2=CCN(C(=O)OC(C)(C)C)CC2)ccc1-c1nnc(-c2ccc(C3=CCN(C(=O)OC(C)(C)C)CC3)cc2)o1]

Synthesis of tert-Butyl 4-{4-[5-(4-{1-[(tert-butoxy)carbonyl.]-1,2,3,6-tetrahydropyridin-4-yl}-2-methylphenyl)-1,3,4-oxadiazol-2-yl]phenyl}-1,2,3,6-tetrahydropyridine-1-carboxylate: A mixture of 2-(4-bromo-2-methylphenyl)-5-(4-bromophenyl)-1,3,4-oxadiazole (100 mg, 0.25 mmol), tert-butyl 4-(4,4,5,5-tetramethyl-1,3,2-dioxaborolan-2-yl)-1,2,3,6-tetrahydropyridine-1-carboxylate (232 mg, 0.75 mmol), dicyclohexyl(2′,6′-dimethoxybiphenyl-2-yl) phosphine (16 mg, 18 μmol), palladium acetate (4.0 mg, 18 mop, potassium carbonate (260 mg, 1.9 mmol), water (0.8 mL) and 1,4-dioxane (2 mL) was purged with nitrogen, stirred and heated to 90° C. for 20 hours. The mixture was diluted with dichloromethane (20 mL) and water. (20 mL), separated and the aqueous layer was extracted with dichloromethane (20 mL). The combined dichloromethane layers were dried (Na₂SO₄) and evaporated. The crude product mixture was purified by silica gel chromatography eluted with a gradient of ethyl acetate in hexanes (25%/50%/75%) to leave a white solid (153 mg, 100%). ¹H-NMR (CDCl₃) δ 8.10 (d, 2H, J=8.5 Hz), 8.02 (d, 1H, J=8.8 Hz), 7.54 (d, 2H, J=8.5 Hz), 7.37 (s, 1H), 7.36 (d, 1H, J=8.8 Hz), 6.20 (m, 2H), 4.15 (m, 4H), 3.65 (m, 4H), 2.77 (s, 3H), 2.58 (m, 4H), 1.50 (s, 18H). LCMS method A R_{f}=7.43 mins, purity >85%, (M+H)⁺=599.

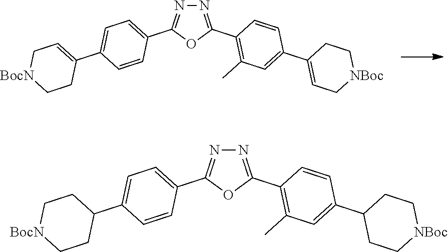 [SMILES: Cc1cc(C2=CCN(C(=O)OC(C)(C)C)CC2)ccc1-c1nnc(-c2ccc(C3=CCN(C(=O)OC(C)(C)C)CC3)cc2)o1 ; Cc1cc(C2CCN(C(=O)OC(C)(C)C)CC2)ccc1-c1nnc(-c2ccc(C3CCN(C(=O)OC(C)(C)C)CC3)cc2)o1]

Synthesis of tert-Butyl 4-{4-[5-(4-{1-[(tert-butoxy)carbonyl]piperidin-4-yl}-2-methylphenyl)-1,3,4-oxadiazol-2-yl]phenyl}piperidine-1-carboxylate: 4-(4-{5-[3-methyl-4-(1,2,3,6-tetrahydropyridin-4-yl)phenyl]-1,3,4-oxadiazol-2-yl}phenyl)-1,2,3,6-tetrahydro pyridine (150 mg, 0.25 mmol) was hydrogenated at 1 atmosphere hydrogen pressure over 5% palladium on carbon (30 mg) in methanol (3 mL) for 24 hours. The reaction mixture was filtered, washed with methanol and evaporated. The product was purified by chromatography on silica gel eluted with a gradient of ethyl acetate in hexanes (25%/50%175%) to leave a foamy solid (120 mg, 80%). ¹H-NMR (CDCl₃) δ 8.06 (d, 2H, J=8.2 Hz), 7.97 (d, 1H, J=8.8 Hz), 7.37 (d, 2H, J=8.2 Hz), 7.20 (s, 1H), 7.19 (d, 1H, J=8.8 Hz), 4.27 (m, 4H), 2.80 (m, 4H), 2.77 (s, 3H), 1.85 (m, 4H), 1.64 (m, 6H), 1.50 (s, 18H). LCMS method A R_{f}==7.41 mins, purity >95%, (M+H)⁺=603.

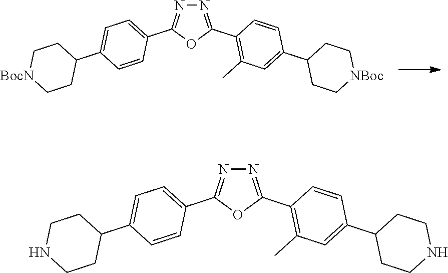 [SMILES: Cc1cc(C2CCN(C(=O)OC(C)(C)C)CC2)ccc1-c1nnc(-c2ccc(C3CCN(C(=O)OC(C)(C)C)CC3)cc2)o1 ; Cc1cc(C2CCNCC2)ccc1-c1nnc(-c2ccc(C3CCNCC3)cc2)o1]

Synthesis of 4-(4-{5-[2-Methyl-4-(piperidin-4-yl)phenyl]-1,3,4-oxadiazol-2-yl}phenyl)piperidine: A solution of tert-butyl 4-{4-[5-(4-{1-[(tert-butoxy)carbonyl]piperidin-4-yl}-2-methylphenyl)-1,3,4-oxadiazol-2-yl]phenyl}piperidine-1-carboxylate (115 mg, 0.19 mmol) in trifluoroacetic acid (1 mL) and dichloromethane (1 mL) was stirred for 2 hours and evaporated. The product was lyophilized to leave a yellow foamy solid (132 mg, 93%).

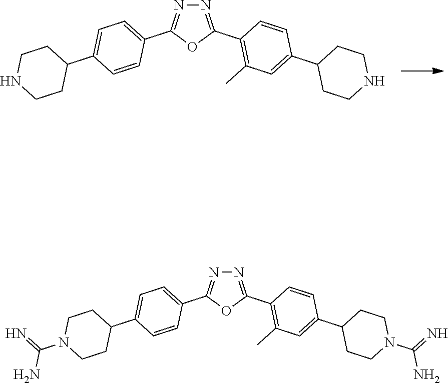 [SMILES: Cc1cc(C2CCNCC2)ccc1-c1nnc(-c2ccc(C3CCNCC3)cc2)o1 ; Cc1cc(C2CCN(C(=N)N)CC2)ccc1-c1nnc(-c2ccc(C3CCN(C(=N)N)CC3)cc2)o1]

### Example 62

Synthesis of 4-(4-{5-[4-(1-Carbamimidoylpiperidin-4-yl)-2-methyl phenyl]-1,3,4-oxadiazol-2-yl}phenyl)piperidine-1-carboximidamide: A solution of 4-(4-{5-[2-methyl-4-(piperidin-4-yl)phenyl]-1,3,4-oxadiazol-2-yl}phenyl)piperidine (125 mg, 0.17 mmol), N—((N′,N″-bis-tert-butyloxycarbonyl)amidino)pyrrazole (156 mg, 0.50 mmol) and N,N-diisopropylethylamine (219 mg, 1.7 mmol, 305 μl) in methanol (2 mL) was stirred for 18 hours. The solvent was evaporated and the crude product was purified by reverse phase HPLC. The product fractions were combined, treated with saturated sodium bicarbonate solution, extracted with methylene chloride (2×50 mL), dried and evaporated to leave product (150 mg, 100%) as a glassy solid. The product was dissolved in trifluoroacetic acid (2 mL) and methylene chloride (2 mL). After stirring for 2 hours the solvents were evaporated, the crude product was purified by preparative reverse phase HPLC and the product fractions were lyophilized to leave a beige solid (91 mg, 65%). ¹H-NMR (CD₃OD) δ 8.10 (d, 2H, J=8.5 Hz), 8.02 (d, 1H, J=7.9 Hz), 7.53 (d, 2H, J=8.5 Hz), 7.36 (s, 1H), 7.33 (d, 1H, J=7.9 Hz), 4.05 (m, 3H), 3.22 (m, 4H), 3.00 (m, 2H), 2.72 (s, 3H), 2.00 (m, 3H), 1.80 (m, 3H). LCMS method A R_{f}=3.09 mins, purity >95%, (M+H)⁺=487.

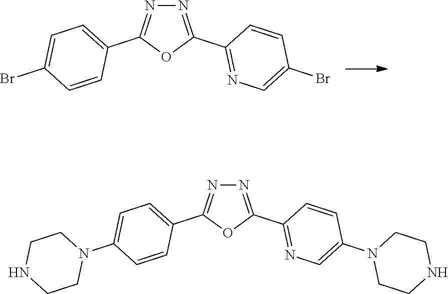 [SMILES: Brc1ccc(-c2nnc(-c3ccc(Br)cn3)o2)cc1 ; c1cc(N2CCNCC2)ccc1-c1nnc(-c2ccc(N3CCNCC3)cn2)o1]

Synthesis of 1-(4-{5-[5-(Piperazin-1-yl)pyridin-2-yl]-1,3,4-oxadiazol-2-yl}phenyl)piperazine: A mixture of 5-bromo-2-[5-(4-bromophenyl)-1,3,4-oxadiazol-2-yl]pyridine (50 mg, 0.13 mmol), N-(tert-butyloxcarbonyl)piperazine (73 mg, 0.39 mmol), dicyclohexyl(2′,6′-dimethoxybiphenyl-2-yl) phosphine (4.4 mg, 2.6 μmol), bispalladium-tri(1,3-dibenzylidene) acetone (1.2 mg (1.3 μmol), cesium carbonate (170 mg, 0.52 mmol) and tolune (1 mL) was purged with nitrogen, stirred and heated to 100° C. for 18 hours. The reaction mixture was cooled to 20° C., diluted with water (25 mL) and extracted with dichloromethane (2×25 mL). The combined extracts were dried (MgSO₄) and evaporated to leave a solid which was purified by silica chromatography eluted with a gradient of ethyl acetate in hexanes (25%50%1100%) to leave a light yellow solid (11 mg, 10%). The solid was dissolved in trifluoroacetic acid (1 mL) and dichloromethane (1 mL) and stood for 2 hours. The solvents were evaporated and the product was lyophilized to leave a tan solid (15 mg, 11%). ¹H-NMR (CD₃OD) δ 8.50 (bs, 1H), 8.16 (d, 1H, J=8.8 Hz), 8.08 (d, 2H, J=9.1 Hz), 7.62 (dd, 1H, J=9.1 Hz, J=2.5 Hz), 7.21 (d, 2H, J=9.1 Hz), 3.70 (m, 4H), 3.62 (m, 4H), 3.42 (m, 8H). LCMS method A R_{f}=2.26 mins, purity >95%, (M+H)⁺=392.

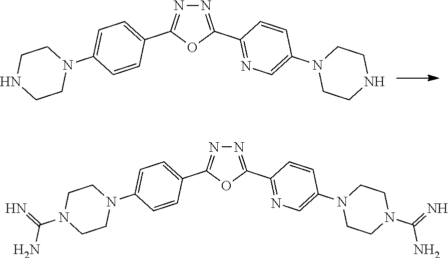 [SMILES: c1cc(N2CCNCC2)ccc1-c1nnc(-c2ccc(N3CCNCC3)cn2)o1 ; N=C(N)N1CCN(c2ccc(-c3nnc(-c4ccc(N5CCN(C(=N)N)CC5)cn4)o3)cc2)CC1]

### Example 63

Synthesis of 4-(4-{5-[5-(4-Carbamimidoylpiperazin-1-yl) pyridin-2-yl]-1,3,4-oxadiazol-2-yl}phenyl)piperazine-1-carboximidamide: A solution of 1-(4-{5-[5-(piperazin-1-yl)pyridin-2-yl]-1,3,4-oxadiazol-2-yl}phenyl)piperazine (14 mg, 17 μmol), N—((N′,N″-bis-tert-butyloxycarbonyl)amidino)pyrrazole (15 mg, 50 μmol) and N,N-diisopropylethylamine (22 mg, 0.17 mmol, 30 μl) in methanol (1 mL) was stirred for 18 hours. N,N-dimethylformamide (1 mL) was added for solubilization and the product was purified by reverse phase HPLC. The product fractions were combined, treated with saturated sodium bicarbonate solution, extracted with methylene chloride (2×50 mL), dried and evaporated to leave product (12 mg, 81%) as a gum. The product was dissolved in 1,4-dioxane (1 mL) and 4N HCl/1,4-dioxane (1 mL). After stirring for 18 hours the solvents were evaporated and the product was lyophilized to leave a beige solid (8.9 mg, 84%). ¹H-NMR (CD₃OD) δ 8.43 (s, 1H), 8.22 (m, 1H), 8.06 (d, 2H, J=9.1 Hz), 7.70 (d, 1H, J=9.4 Hz), 7.14 (d, 2H, J=9.1 Hz), 3.75 (m, 12H), 3.70 (bs, 6H), 3.55 (m, 4H). LCMS method A R_{f}=2.67 mins, purity >90%, (M+H)⁺=476.

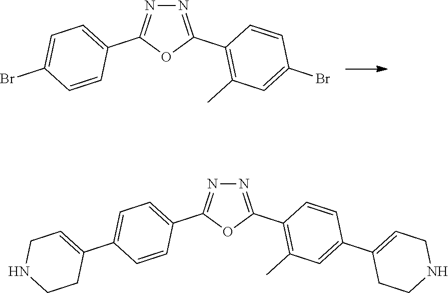 [SMILES: Cc1cc(Br)ccc1-c1nnc(-c2ccc(Br)cc2)o1 ; Cc1cc(C2=CCNCC2)ccc1-c1nnc(-c2ccc(C3=CCNCC3)cc2)o1]

Synthesis of 4-(3-Methyl-4-{5-[4-(1,2,3,6-tetrahydropyridin-4-yl)phenyl]-1,3,4-oxadiazol-2-yl}phenyl)-1,2,3,6-tetrahydropyridine: A mixture of 2-(4-bromo-2-methyl phenyl)-5-(4-bromophenyl)-1,3,4-oxadiazole (100 mg, 0.25 mmol), tert-butyl 4-(4,4,5,5-tetramethyl-1,3,2-dioxaborolan-2-yl)-1,2,3,6-tetrahydropyridine-1-carboxylate (232 mg, 0.75 mmol), dicyclohexyl(2′,6′-dimethoxybiphenyl-2-yl) phosphine (16 mg, 18 μmol), palladium acetate (4.0 mg, 18 μmol), potassium carbonate (260 mg, 1.9 mmol), water (0.8 mL) and 1,4-dioxane (2 mL) was purged with nitrogen, stirred and heated to 90° C. for 20 hours. The mixture was diluted with dichloromethane (20 mL) and water (20 mL), separated and the aqueous layer was extracted with dichloromethane (20 mL). The combined dichloromethane layers were dried (Na₂SO₄) and evaporated. The crude product mixture was dissolved in trifluoroacetic acid (1 mL) and dichloromethane (1 mL) and stirred for 2 h. The solvents were evaporated and the crude product was purified by preparative reverse phase HPLC and the product fractions were combined and lyophilized to leave a white solid (111 mg, 60%). ¹H-NMR (CD₃OD) δ 8.17 (d, 2H, J=8.5 Hz), 8.08 (d, 1H, J=8.2 Hz), 7.74 (d, 2H, J=8.5 Hz), 7.57 (s, 1H), 7.55 (d, 1H, J=8.2 Hz), 6.35 (m, 2H), 3.90 (m, 4H), 3.52 (m, 4H), 2.86 (m, 4H), 2.78 (s, 3H). LCMS method A R_{f}=2.69 mins, purity >95%, (M+H)⁺=399.

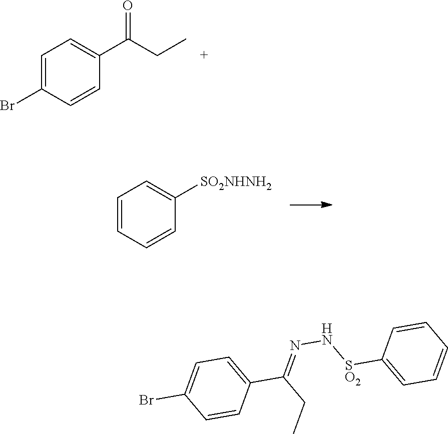

Synthesis of N′-[(1E)-1-(4-Bromophenyl)ethylidene]benzenesulfonohydrazide: Phenylsulfonylhydrazide (2.1 g, 12 mmol) was dissolved in methanol (10 mL) and the solution was treated with 4-bromopropioophenone (2.4 g, 12 mmol). After stirring for 30 minutes a thick precipitate had formed. Additional methanol (10 mL) was added and the mixture was filtered, washed with methanol and dried under vacuum to leave 3.6 g (85%) of white solid which was a mixture of syn and anti isomers. ¹H-NMR (CDCl₃) δ 8.03 (d, 1H, J=8.5 Hz), 7.92 (d, 0.5H, 3=8.5 Hz), 7.82 (d, 0.5H, J=8.5 Hz), 7.45-7.65 (m, 7H), 2.60 and 2.15 (s, 3H). LCMS method A R_{f}=5.30 mins, purity >95%, (M+H)⁺=354.

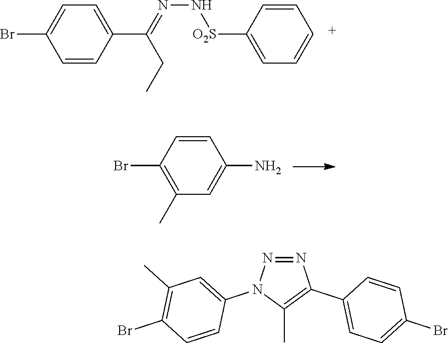

Synthesis of 1-(4-Bromo-3-methylphenyl)-4-(4-bromophenyl)-5-methyl-1H-1,2,3-triazole: A mixture of N′-[(1E)-1-(4-bromophenyl)ethylidene]benzenesulfonohydrazide (530 mg, 1.5 mmol), 4-bromo-3-methylaniline (561 mg, 3.0 mmol), copper(II) acetate (273 mg, 1.5 mmol) and pivalic acid (3.5 mg, 3.0 mmol) in toluene (15 mL) was stirred and heated to 100° C. for 18 hours open to the atmosphere. The mixture was cooled to 20° C., diluted with dichloromethane (15 mL) and evaporated onto silica gel. The crude product was purified by silica gel chromatography eluted with a gradient of 50% hexanes in dichloromethane to 100% dichloromethane to leave the product as a tan solid (78 mg, 12%). ¹H-NMR (CDCl₃) δ 7.75-7.50 (m, 6H), 7.16 (d, 1H, 3=8.2 Hz), 2.34 (s, 3H), 2.08 (s, 3H). LCMS method A R_{f}=6.19 mins, purity >95%, (M+H)⁺=408.

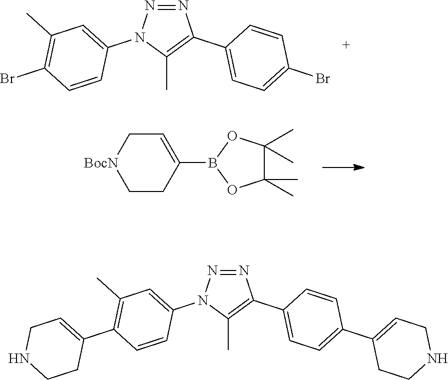

Synthesis of 4-(2-Methyl-4-(5-methyl-4-[4-(1,2,3,6-tetrahydropyridin-4-yl)phenyl]-1H-1,2,3-triazol-1-yl)phenyl)-1,2,3,6-tetrahydropyridine: A mixture of 1-(4-bromo-3-methylphenyl)-4-(4-bromophenyl)-5-methyl-1H-1,2,3-triazole (50 mg, 0.12 mmol), tert-butyl 4-(4,4,5,5-tetramethyl-1,3,2-dioxaborolan-2-yl)-1,2,3,6-tetrahydropyridine-1-carboxylate (93 mg, 0.30 mmol), dicyclohexyl (2′,6′-dimethoxybiphenyl-2-yl) phosphine (8 mg, 20 μmol), palladium acetate (2.0 mg, 7 μmol), potassium carbonate (110 mg, 0.80 mmol), water. (0.4 mL) and 1,4-dioxane (1 mL) was purged with nitrogen, stirred and heated to 90° C. for 20 hours. The mixture was diluted with dichloromethane (20 mL) and water (20 mL) and the aqueous layer was extracted with dichloromethane (20 mL). The combined dichloromethane layers were dried (Na₂SO₄) and evaporated. The crude product mixture was purified by silica gel chromatography eluted with a gradient of 15% ethyl acetate in hexanes to 50% ethyl acetate in hexanes to leave the product as a gum (21 mg, 29%). This was dissolved in trifluoroacetic acid (1 mL) and dichloromethane (1 mL) and stirred for 2 hours. The mixture was evaporated and lyophilized to leave the product as a white solid (bis trifluoroacetic acid salt, 18 mg, 60%). ¹H-NMR (CD₃OD) δ 7.78 (d, 2H, J=8.2 Hz), 7.63 (d, 2H, J=8.2 Hz), 7.49 (s, 1H), 7.44 (d, 1H, J=8.5 Hz), 7.42 (d, 1H. J=8.5 Hz), 6.25 (m, 1H, 5.77 (m, 1H), 3.89 (m, 4H), 3.52 (m, 4H), 2.85 (m, 2H), 2.65 (m, 2H), 2.48 (s, 3H), 2.42 (s, 3H). LCMS method A R_{f}=2.67 mins, purity >95%, (M+H)⁺=412.

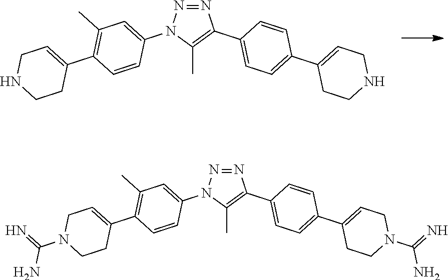 [SMILES: Cc1cc(-n2nnc(-c3ccc(C4=CCNCC4)cc3)c2C)ccc1C1=CCNCC1 ; Cc1cc(-n2nnc(-c3ccc(C4=CCN(C(=N)N)CC4)cc3)c2C)ccc1C1=CCN(C(=N)N)CC1]

### Example 65

Synthesis of 4-(4-{4-[4-(1-Carbamimidoyl-1,2,3,6-tetrahydropyridin-4-yl)phenyl]-5-methyl-1H-1,2,3-triazol-1-yl}-2-methylphenyl)-1,2,3,6-tetrahydropyridine-1-carboximidamide: A solution of 4-(2-methyl-4-(5-methyl-4-[4-(1,2,3,6-tetrahydropyridin-4-yl) phenyl]-1H-1,2,3-triazol-1-yl)phenyl)-1,2,3,6-tetrahydropyridine (11 mg, 16 μmol) and N,N-diisopropylethylamine (20 mg, 0.16 mmol) in methanol (1 mL) was treated with N—((N′, N″-bis-tert-butyloxycarbonyl)amidino)pyrrazole (15 mg, 48 μmol) and stirred for 18 hours. N,N-dimethyl formamide (1 mL) was added and the mixture was purified by reversed phase HPLC. The product fractions were combined, treated with saturated sodium bicarbonate solution and extracted with dichloromethane (2×25 mL). The combined organic layers were dried (Na₂SO₄) and evaporated to a glassy solid which was dissolved in trifluoroacetic acid (1 mL) and Dichloromethane (1 mL) and stirred for 2 hours. The solvents were evaporated and the product was lyophilized to a white solid (4.4 mg, 38%). ¹H-NMR (CD₃OD) δ 7.77 (d, 2H, J=8.3 Hz), 7.63 (d, 2H, J=8.3 Hz), 7.47 (s, 1H), 7.45 (d, 1H, J=8.5 Hz), 7.42 (d, 1H, J=8.5 Hz), 6.27 (m, 1H), 5.80 (m, 1H), 3.76 (m, 4H), 3.48 (m, 4H), 2.85 (m, 2H), 2.66 (m, 2H), 2.50 (s, 3H), 2.44 (s, 3H). LCMS method A R_{f}=2.52 mins, purity >95%, (M+H)⁺=496.

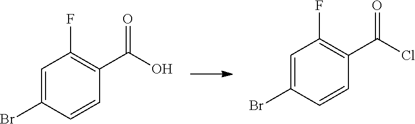 [SMILES: O=C(O)c1ccc(Br)cc1F ; O=C(Cl)c1ccc(Br)cc1F]

Synthesis of 4-bromo-2-fluoro-benzoyl chloride: To a suspension of 4-bromo-2-fluoro-benzoic acid (10.0 g, 45.7 mmol) and N,N-dimethylformamide (0.334 g, 0.352 mL, 4.57 mmol) in CHCl₃ (114 mL) in a 1-liter round bottom flask at 0° C. under N₂ was added oxalyl chloride (8.69 g, 5.87 mL, 68.5 mmol) dropwise via an addition funnel over 30 minutes. The cooling bath warmed to 23° C. and stirred for 18 hours. The mixture was concentrated. The residue was suspended in CHCl₃ and concentrated again, then dried under vacuum to yield 4-bromo-2-fluoro-benzoyl chloride, which was used without purification in the next reaction.

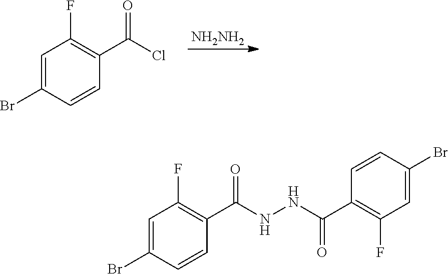

Synthesis of 4-bromo-2-fluoro-benzoic acid N′-(4-bromo-2-fluoro-benzoyl)-hydrazide: To a solution of hydrazine hydrate (0.732 g, 1.11 mL, 22.9 mmol) and N,N-diisopropylethylamine (7.68 g, 10.3 mL, 59.4 mmol) in CHCl₃ (50 mL) in a 1-liter round bottom flask at 0° C. under N₂ was added a solution of 4-bromo-2-fluoro-benzoyl chloride (45.7 mmol) in CHCl₃ (60 mL) dropwise via an addition funnel over 60 minutes. The cooling bath warmed to 23° C. and stirred for 18 hours. The mixture was concentrated. The residue was suspended in water (0.150 mL), then stirred at 23° C. for 18 hours. The water was decanted; additional water (150 mL) was added and decanted after stirring briefly. Ether (30 mL) and acetonitrile (120 mL) were added, and then the mixture was concentrated to dryness (2×), then dried under vacuum to yield 4-bromo-2-fluoro-benzoic acid N′-(4-bromo-2-fluoro-benzoyl)-hydrazide (8.93 g, 90% over two steps) as an

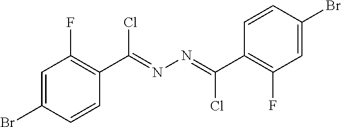 [SMILES: Fc1cc(Br)ccc1/C(Cl)=N/N=C(\Cl)c1ccc(Br)cc1F]

Synthesis of (Z)-4-bromo-N—[(Z)-(4-bromo-2-fluorophenyl)(chloro)methylidene]-2-fluorobenzene-1-carbohydrazonoyl chloride: To a suspension of 4-bromo-2-fluoro-benzoic acid N′-(4-bromo-2-fluoro-benzoyl)-hydrazide (0.350 g, 0.806 mmol) in anhydrous toluene (9 mL) in a pressure tube at 23° C. under N₂ was gradually added PCl₅ (1.13 g, 5.4.1 mmol). The mixture was heated at 95° C. for 18 hours. The reaction was cooled to 23° C. and concentrated, then suspended in toluene and concentrated again to yield (Z)-4-bromo-N—[(Z)-(4-bromo-2-fluorophenyl)(chloro) methylidene]-2-fluorobenzene-1-carbohydrazonoyl chloride (0.3731 g, 98%) as a yellow solid, which was used without purification in the next reaction.

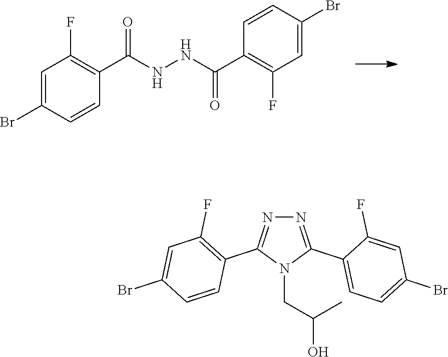 [SMILES: O=C(NNC(=O)c1ccc(Br)cc1F)c1ccc(Br)cc1F ; CC(O)Cn1c(-c2ccc(Br)cc2F)nnc1-c1ccc(Br)cc1F]

Synthesis of 1-[3,5-bis-(4-bromo-2-fluoro-phenyl)-[1,2,4]triazol-4-yl]-propan-2-ol: To a solution of (Z)-4-bromo-N—[(Z)-(4-bromo-2-fluorophenyl)(chloro)methylidene]-2-fluorobenzene-1-carbohydrazonoyl chloride (0.450 g, 0.96 mmol) in methanol (10 mL) was added 1-amino-propan-2-ol (0.128 g, 1.71 mmol) dropwise over one minute. After five minutes, N,N-diisopropylethylamine (0.477 g, 0.680 mL, 3.17 mmol) was added dropwise over one minute. After stirring for 30 minutes at 23° C., the reaction was heated at 65° C. for 18 hours. The mixture was cooled to 23° C., acetic acid (4 mL) was added, and the reaction was heated at 80° C. for two hours. The reaction was then cooled to 23° C., concentrated, then partitioned between ethyl acetate (30 mL) and water (20 mL) and separated. The organic layer was dried and concentrated to provide the title compound.

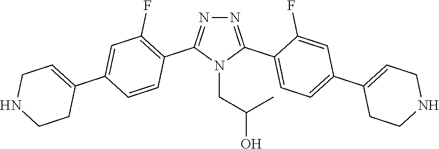 [SMILES: CC(O)Cn1c(-c2ccc(C3=CCNCC3)cc2F)nnc1-c1ccc(C2=CCNCC2)cc1F]

Synthesis of 1-{3,5-bis-[2-fluoro-4-(1,2,3,6-tetrahydro-pyridin-4-yl)-phenyl]-[1,2,4]triazol-4-yl}-propan-2-ol trifluoroacetate: A mixture of 1-[3,5-bis-(4-bromo-2-fluoro-phenyl)-[1,2,4]triazol-4-yl]-propan-2-ol (0.426 g, 0.90 mmol), tert-butyl 5,6-dihydro-4-(4,4,5,5-tetramethyl-1,3,2-dioxaborolan-2-yl)pyridine-1(2H)-carboxylate (0.640 g, 2.07 mmol), 2 M K₂CO₃ solution (3.2 mL, 6.30 mmol), 2-dicyclohexylphosphino-2′,6′-dimethoxybiphenyl (0.074 g, 0.180 mmol), and Pd(OAc)₂ (0.016 g, 0.072 mmol) in 1,4-dioxane (5 mL) was degassed by bubbling nitrogen through the mixture for five minutes. The reaction was heated at 95° C. for 18 hours. The mixture was cooled to 23° C., then partitioned between ethyl acetate (40 mL) and water (20 mL) and separated. The organic layer was dried and concentrated to yield a brown solid. This residue was dissolved in CH₂Cl₂ (4 mL) and trifluoroacetic acid (6 mL) was added dropwise over five minutes. After stirring at 23° C. for two hours, the reaction was concentrated. The residue was triturated with ether (20 mL), then used without further purification in the next reaction.

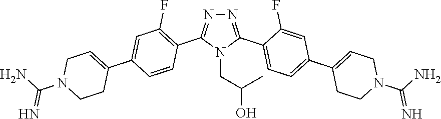 [SMILES: CC(O)Cn1c(-c2ccc(C3=CCN(C(=N)N)CC3)cc2F)nnc1-c1ccc(C2=CCN(C(=N)N)CC2)cc1F]

### Example 66

Synthesis of 4-(4-{5-[4-(1-carbamimidoyl-1,2,3,6-tetrahydropyridine-4-yl)-2-fluorophenyl]-4-(2-hydroxypropyl)-4H-1,2,4-triazol-3-yl}-3-fluorophenyl)-1,2,3,6-tetrahydropyridine-1-carboximidamide trifluoroacetate: A mixture of 1-{3,5-bis-[2-fluoro-4-(1,2,3,6-tetrahydro-pyridin-4-yl)-phenyl]-[1,2,4]triazol-4-yl}-propan-2-ol trifluoroacetate (0.430 g, 0.60 mmol) and triethylamine (0.424 g, 0.610 mL, 4.2 mmol) in N,N-dimethylformamide (3 mL) at 23° C. was stirred for 10 minutes before adding tert-butyl N—[(Z)-{[(tert-butoxy)carbonyl]imino}(1H-pyrazol-1-yl)methyl]carbamate (0.450 g, 1.44 mmol). The reaction was stirred at 23° C. for 18 hours. Additional tert-butyl N—[(Z)-{[(tert-butoxy)carbonyl]imino}(1H-pyrazol-1-yl)methyl]carbamate (0.200 g, 0.645 mmol) was added, and the reaction was stirred for four hours, then partitioned between ethyl acetate (40 mL) and water (30 mL) and separated. The organic layer was washed with additional water. (2×30 mL), then dried and concentrated. Silica gel chromatography (0 to 100% (20% isopropanol-80% CH₂Cl₂); 100 to 0% CH₂Cl₂), followed by concentration and drying under vacuum, yielded 0.500 g of a solid. This solid was dissolved in CH₂Cl₂ (4 mL) and trifluoroacetic acid (5 mL) was added. After stirring at 23° C. for 90 minutes, the reaction was concentrated. The residue was purified by reverse-phase HPLC and lyophilized to yield the title compound (0.112 g, 24%). ¹H NMR (300 MHz, DMSO-d₆) δ=7.71-7.62 (m, 2H), 7.58 (d, J=11.7 Hz, 2H), 7.55-7.50 (m, 2H), 7.45 (s, 8H), 6.47 (br s, 2H), 4.86 (br s, 1H), 4.10 (br s, 4H), 3.87-3.67 (m, 2H), 3.63 (br t, J=5.3 Hz, 4H), 3.31-3.25 (m, 2H), 2.63 (br s, 4H), 0.65 (d, J=5.9 Hz, 3H). LC/MS (M+H)⁺=562.

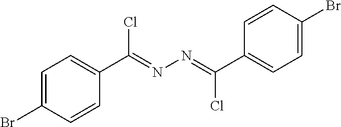 [SMILES: Cl/C(=N\N=C(/Cl)c1ccc(Br)cc1)c1ccc(Br)cc1]

Synthesis of (Z)-4-bromo-N—[(Z)-(4-bromophenyl)(chloro)methylidene]benzene-1-carbohydrazonoyl chloride: The title compound was prepared according to the procedure of (Z)-4-bromo-N—[(Z)-(4-bromo-2-fluorophenyl)(cloro)methylidene]-2-fluorobenzene-1-carbohydrazonoyl chloride except that 4-bromo-2-fluoro-benzoic acid N′-(4-bromo-2-fluoro-benzoyl)-hydrazide was replaced with 4-bromo-N-(4-bromobenzoyl)benzohydrazide.

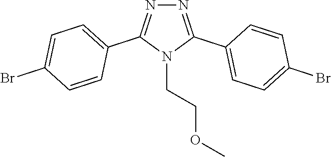 [SMILES: COCCn1c(-c2ccc(Br)cc2)nnc1-c1ccc(Br)cc1]

Synthesis of 3,5-bis-(4-bromo-phenyl)-4-(2-methoxy-ethyl)-4H-[1,2,4]triazole: To a slurry of (Z)-4-bromo-N—[(Z)-(4-bromophenyl)(chloro)methylidene]benzene-1-carbohydrazonoyl chloride (0.840 g, 1.94 mmol) in methanol (5 mL) was added 2-methoxy-ethylamine (0.320 g, 4.26 mmol) dropwise over one minute, followed by N,N-diisopropylethylamine (0.750 g, 1.10 mL, 5.62 mmol). After stirring for 15 minutes at 23° C., the reaction was heated at 50° C. for one hour. N,N-dimethylformamide (2 mL) was added, and the temperature was increased to 60° C. for an additional 60 minutes. The mixture was cooled, acetic acid (3 mL) was added dropwise over three minutes, and the reaction was heated at 85° C. for two hours. After cooling to 23° C., water (15 mL) was added, and the mixture was left standing at 23° C. for 18 hours. The precipitate was collected by filtration, washed with water (5 mL) and ether (15 mL), and air dried for 90 minutes to yield the title compound (0.590 g, 70%).

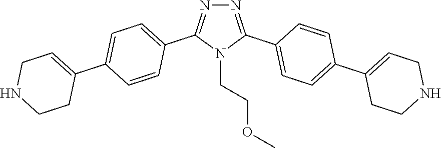 [SMILES: COCCn1c(-c2ccc(C3=CCNCC3)cc2)nnc1-c1ccc(C2=CCNCC2)cc1]

Synthesis of 4-{4-[4-(2-methoxyethyl)-5-[4-(1,2,3,6-tetrahydropyridin-4-yl)phenyl]-4H-1,2,4=triazol-3-yl]phenyl}-1,2,3,6-tetrahydropyridine trifluoroacetate: A mixture of 3,5-bis-(4-bromo-phenyl)-4-(2-methoxy-ethyl)-4H-[1,2,4]triazole (1.70 mmol), tert-butyl 5,6-dihydro-4-(4,4,5,5-tetramethyl-1,3,2-dioxaborolan-2-yl)pyridine-1(2H)-carboxylate (1.160 g, 3.74 mmol), 2 M K₂CO₃ solution (6.40 mL, 12.8 mmol), 2-dicyclohexylphosphino-2′,6′-dimethoxybiphenyl (0.137 g, 0.340 mmol), and Pd(OAc)₂ (0.031 g, 0.136 mmol) in 1,4-dioxane (8 mL) was degassed by bubbling nitrogen through the mixture. The reaction was heated at 95° C. for 18 hours. The mixture was cooled to 23° C., then diluted with ethyl acetate (5 mL) and water (15 mL). After brief sonication, the solid was collected by filtration, washed with water (5 mL) and ether (15 mL), then dissolved in CH₂Cl₂ (5 mL). Anisole (0.500 mL) was added, followed by dropwise addition of trifluoroacetic acid (10 mL), and the mixture was stirred at 23° C. for 2.5 hours. The reaction was concentrated; the residue was triturated with ether (2×30 mL), then dried under vacuum to yield the title compound.

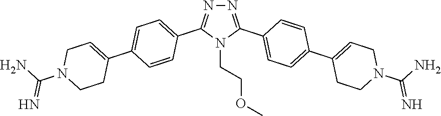 [SMILES: COCCn1c(-c2ccc(C3=CCN(C(=N)N)CC3)cc2)nnc1-c1ccc(C2=CCN(C(=N)N)CC2)cc1]

### Example 67

Synthesis of 4-(4-{5-[4-(1-carbamimidoyl-1,2,3,6-tetrahydropyridin-4-yl)phenyl]-4-(2-methoxyethyl)-4H-1,2,4-triazol-3-yl}phenyl)-1,2,3,6-tetrahydropyridine-1-carboximidamide trifluoroacetate: To a solution of 4-{4-[4-(2-methoxyethyl)-5[4-(1,2,3,6-tetrahydro pyridin-4-yl)phenyl]-4H-1,2,4-triazol-3-yl]phenyl}-1,2,3,6-tetrahydropyridine trifluoroacetate (0.90 mmol) in a mixture of N,N-dimethylformamide (4 mL) and CH₂Cl₂ (2 mL) was added triethylamine (0.640 g, 6.30 mmol) dropwise over 1 minute. After five minutes, tert-butyl N—[(Z)-{[(tert-butoxy)carbonyl]imino}(1H-pyrazol-1-yl)methyl]carbamate (0.640 g, 2.07 mmol) was added. The reaction was stirred at 23° C. for four days. The mixture was diluted with ethyl acetate (30 mL) and water (20 mL) and separated. The organic layer was washed with additional water (2×20 mL), then dried and concentrated. Silica gel chromatography (0 to 100% (20% MeOH-80% CH₂Cl₂); 100 to 0% CH₂Cl₂), followed by concentration and drying under vacuum, yielded an oil. The oil was dissolved in CH₂Cl₂ (5 mL) and anisole (0.700 mL) was added, followed by trifluoroacetic acid (8 mL). After stirring at 23° C. for 18 hours, the reaction was concentrated. The residue was triturated with ether (30 mL), and the residue was purified by reverse-phase HPLC (5 to 40% MeCN-water (both with 0.1% TFA)) and lyophilized to yield the title compound (0.082 g, 12%). ¹H NMR (300 MHz, DMSO-d₆) δ=7.71 (q, J=8.8 Hz, 8H), 7.48 (br s, 8H), 6.38 (br 5, 2H), 4.33 (br t, J=4.7 Hz, 2H), 4.11 (br s, 4H), 3.65 (br t, J==5.6 Hz, 7H), 3.17 (t, J=5.6 Hz, 2H), 2.87 (s, 3H), 2.65 (br s, 4H). LC/MS (M+H)⁺=526.

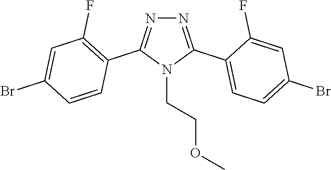 [SMILES: COCCn1c(-c2ccc(Br)cc2F)nnc1-c1ccc(Br)cc1F]

Synthesis of 3,5-bis-(4-bromo 2-fluoro-phenyl)-4-(2-methoxy-ethyl)-4H-[1,2,4]triazole: To a solution of (Z)-4-bromo-N—[(Z)-(4-bromo-2-fluorophenyl)(chloro)methylidene]-2-fluorobenzene-1-carbohydrazonoyl chloride (3.46 mmol) in anhydrous toluene (17 mL) at 0° C. under N₂ was added 2-methoxy-ethylamine (2.60 g, 2.98 mL, 34.6 mmol) dropwise. The reaction was stirred at 0° C. for two hours, then the cooling was permitted to warm gradually to 23° C. for 18 hours. Acetic acid (5 mL) was added dropwise, and the mixture was heated to 60° C. for 90 minutes before cooling to 23° C. and concentrating. The residue was partitioned between ethyl acetate (250 mL) and saturated NaHCO₃ solution (35 mL) and separated. The organic layer was washed with water. (2×25 mL), and brine (25 mL), then dried (Na₂SO₄), filtered, and concentrated. Silica gel chromatography (0 to 100% ethyl acetate; 100 to 0% hexanes), followed by concentration and drying under vacuum, yielded the title compound (1.3094 g, 80%) as a white solid.

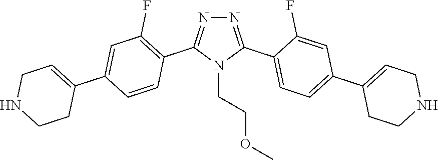 [SMILES: COCCn1c(-c2ccc(C3=CCNCC3)cc2F)nnc1-c1ccc(C2=CCNCC2)cc1F]

Synthesis of 4-(3-fluoro-4-{5-[2-fluoro-4-(1,2,3,6-tetrahydropyridin-4-yl)phenyl]-4-(2-methoxyethyl)-4H-1,2,4-triazol-3-yl}phenyl)-1,2,3,6-tetrahydropyridine trifluoroacetate: A mixture of 3,5-bis-(4-bromo-2-fluoro-phenyl)-4-(2-methoxy-ethyl)-4H-[1,2,4]triazole (1.00 mmol), tert-butyl 5,6-dihydro-4-(4,4,5,5-tetramethyl-1,3,2-dioxaborolan-2-yl)pyridine-1(2H)-carboxylate (0.682 g, 2.20 mmol), 2-dicyclohexylphosphino-2′,6′-dimethoxybiphenyl (0.082 g, 0.200 mmol), and Pd(OAc)₂ (0.018 g, 0.080 mmol) in 1,4-dioxane (5 mL) was degassed by bubbling nitrogen through the mixture for several minutes. After adding 2 M K₂CO₃ solution (3.8 mL, 7.50 mmol), the reaction was heated at 100° C. for 18 hours. The mixture was cooled to 23° C. and partitioned between ethyl acetate (30 mL) and water (15 mL) and separated. The organic layer was washed with water (25 mL), then dried and concentrated to yield an oil. This residue was dissolved in CH₂Cl₂ (4 mL) and 1,3-dimethoxybenzene (0.400 mL) was added, followed by dropwise addition of trifluoroacetic acid (8 mL) over two minutes. After stirring at 23° C. for 18 hours, the reaction was concentrated. The residue was triturated with ether (30 mL), then purified by reverse-phase HPLC to yield the title compound.

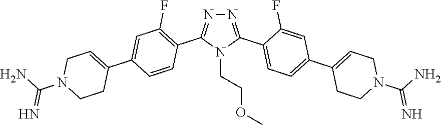 [SMILES: COCCn1c(-c2ccc(C3=CCN(C(=N)N)CC3)cc2F)nnc1-c1ccc(C2=CCN(C(=N)N)CC2)cc1F]

### Example 68

Synthesis of 4-(4-{5-[4-(1-carbamimidoyl-1,2,3,6-tetrahydropyridin-4-yl)-2-fluorophenyl]-4-(2-methoxyethyl)-4H-1,2,4-triazol-3-yl}-3-fluorophenyl)-1,2,3,6-tetrahydropyridine-1-carboximidamide trifluoroacetate: To a solution of 4-(3-fluoro-4-{5-[2-fluoro-4-(1,2,3,6-tetrahydropyridin-4-yl)phenyl]-4-(2-methoxyethyl)-4H-1,2,4-triazol-3-yl}phenyl)-1,2,3,6-tetrahydropyridine trifluoroacetate (0.80 mmol) and triethylamine (0.810 mL, 5.60 mmol) in a mixture of N,N-dimethylformamide (4 mL) and CH₂Cl₂ (2 mL) was added tert-butyl N—[(Z)-{[(tert-butoxy)carbonyl] imino}(1H-pyrazol-1-yl)methyl]carbamate (0.570 g, 1.84 mmol). The reaction was stirred at 23° C. for 72 hours. The mixture was diluted with ethyl acetate (30 mL) and water (20 mL) and separated. The organic layer was washed with additional water (2×20 mL), then dried and concentrated. Silica gel chromatography (0 to 100% (20% MeOH-80% CH₂Cl₂); 100 to 0% CH₂Cl₂), followed by concentration and drying under vacuum, yielded the desired intermediate, which was deprotected according to the procedure of 4-(4-{5-[4-(1-carbamimidoyl-1,2,3,6-tetrahydropyridin-4-yl)-2-fluorophenyl]-4-methyl-4H-1,2,4-triazol-3-yl}-2-fluorophenyl)-1,2,3,6-tetrahydropyridine-1-carboximidamide trifluoroacetate. The crude material was purified by reverse-phase HPLC and lyophilized to yield the title compound (0.020 g, 3%). ¹H NMR (300 MHz, DMSO-d₆) δ=7.71-7.63 (m, 2H), 7.60 (d, J=12.3 Hz, 2H), 7.56-7.43 (m, 10H), 6.48 (br s, 2H), 4.12 (br s, 4H), 4.03 (br t, J=4.1 Hz, 2H), 3.64 (br t, J=5.6 Hz, 4H), 3.1.1 (t, J=5.0 Hz, 2H), 2.83 (s, 3H), 2.64 (br s, 4H). LC/MS (M+H)⁺=562.

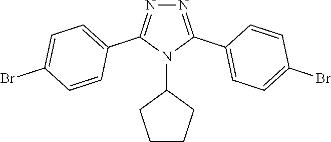 [SMILES: Brc1ccc(-c2nnc(-c3ccc(Br)cc3)n2C2CCCC2)cc1]

Synthesis of 3,5-bis-(4-bromo-phenyl)-4-cyclopentyl-4H-[1,2,4]triazole: To a solution of (Z)-4-bromo-N—[(Z)-(4-bromophenyl)(chloro)methylidene]benzene-1-carbohydrazonoyl chloride (0.810 g, 1.86 mmol) in methanol (5 mL) was added cyclopentylamine (0.348 g, 4.10 mmol) dropwise over one minute, followed by N,N-diisopropylethylaminde (0.720 g, 1.030 mL, 5.58 mmol) five minutes later. After stirring for 30 minutes at 23° C., the reaction was heated at 55° C. for 18 hours. The reaction was cooled to 23° C., acetic acid (3 mL) was added, and the mixture was heated at 85° C. for three hours, then 100° C. for one hour. The reaction was cooled, then diluted with water (25 mL) and kept at 23° C. for seven days. The precipitated solid was collected by filtration, washed with water (10 mL) and ether (15 mL), then air dried for 18 hours to yield the title compound (0.650 g, 78%).

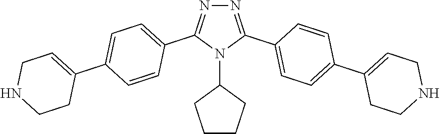 [SMILES: C1=C(c2ccc(-c3nnc(-c4ccc(C5=CCNCC5)cc4)n3C3CCCC3)cc2)CCNC1]

Synthesis of 4-(4-{4-cyclopentyl-5-[4-(1,2,3,6-tetrahydropyridin-4-yl)phenyl]-4H-1,2,4-triazol-3-yl}phenyl)-1,2,3,6-tetrahydropyridine trifluoroacetate: A mixture of 3,5-bis-(4-bromo-phenyl)-4-cyclopentyl-4H-[1,2,4]triazole 1.65 mmol), tert-butyl 5,6-dihydro-4-(4,4,5,5-tetramethyl-1,3,2-dioxaborolan-2-yl)pyridine-1(2H)-carboxylate (1.150 g, 3.71 mmol), 2 M K₂CO₃ solution (6.20 mL, 12.3 mmol), 2-dicyclohexylphosphino-2′,6′-dimethoxybiphenyl (0.135 g, 0.330 mmol), and Pd(OAc)₂ (0.030 g, 0.170 mmol) in 1,4-dioxane (9 mL) was degassed by bubbling nitrogen through the mixture for five minutes. The reaction was heated at 105° C. for 24 hours. The mixture was cooled to 23° C., then diluted with ethyl acetate (20 mL) and water (15 mL) and separated. The organic layer was dried and concentrated to yield an oil, which was triturated with ether (20 mL). The resulting solid was collected and air dried, then dissolved in CH₂Cl₂ (5 mL). The solution was treated with anisole (0.600 mL), then trifluoroacetic acid (2 mL) was added dropwise over three minutes. The mixture was stirred at 23° C. for 18 hours. The reaction was then concentrated, triturated with ether (25 mL), and purified by reverse-phase HPLC to yield the title compound.

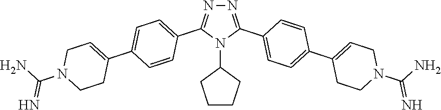 [SMILES: N=C(N)N1CC=C(c2ccc(-c3nnc(-c4ccc(C5=CCN(C(=N)N)CC5)cc4)n3C3CCCC3)cc2)CC1]

### Example 69

Synthesis of 4-(4-{5-[4-(1-carbamimidoyl-1,2,3,6-tetrahydropyridin-4-yl)phenyl]-4-cyclopentyl-4H-1,2,4-triazol-3-yl}phenyl)-1,2,3,6-tetrahydropyridine-1-carboximidamide trifluoroacetate: To a solution of 4-(4-{4-cyclopentyl-5-[4-(1,2,3,6-tetrahydropyridin-4-yl) phenyl]-4H-1,2,4-triazol-3-yl}phenyl)-1,2,3,6-tetrahydropyridine trifluoroacetate (0.88 mmol) and triethylamine (0.601 g, 0.860 mL, 5.95 mmol) in a mixture of N,N-dimethylformamide (5 mL) and CH₂Cl₂ (2 mL) was added tert-butyl N—[(Z)-{[(tert-butoxy)carbonyl]imino}(1H-pyrazol-1-yl)methyl]carbamate (0.560 g, 1.80 mmol). The reaction was stirred at 23° C. for 18 hours. The mixture was diluted with ethyl acetate (20 mL) and water (20 mL) and separated. The organic layer was washed with additional water (2×30 mL), then dried and concentrated. Silica gel chromatography (0 to 100% (20% MeOH-80% CH₂Cl₂); 100 to 0% CH₂Cl₂), followed by concentration and drying under vacuum, yielded the desired intermediate. This material was dissolved in CH₂CL (4 mL), and anisole (0.500 mL) and trifluoroacetic acid (6 mL) were then added. After two hours, the mixture was concentrated and purified by reverse-phase HPLC (5 to 45% MeCN-water (both with 0.1% trifluoroacetic acid) over 17 min.). The good fractions were combined and lyophilized to yield the desired compound (0.120 g, 18%). ¹H NMR (300 MHz, DMSO-d₆) δ=7.71-7.60 (m, J==8.4, 8.4, 8.4 Hz, 8H), 7.48 (s, 8H), 6.38 (br s, 2H), 4.60-4.43 (m, 1H), 4.12 (br s, 4H), 3.65 (br t, J=5.6 Hz, 4H), 2.65 (br s, 4H), 2.06-1.89 (m, 2H), 1.85-1.66 (m, 2H), 1.41-1.14 (m, 4H). LC/MS (M+H)⁺=536.

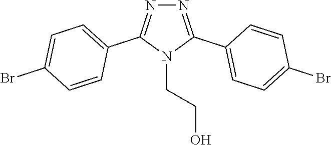 [SMILES: OCCn1c(-c2ccc(Br)cc2)nnc1-c1ccc(Br)cc1]

Synthesis of 2-[3,5-bis-(4-bromo-phenyl)-[1,2,4]triazol-4-yl]-ethanol: To a solution of (Z)-4-bromo-N—[(Z)-(4-bromophenyl)(chloro)methylidene]benzene-1-carbohydrazonoyl chloride (0.1.70 g, 3.91 mmol) in methanol (15 mL) was added 2-amino-ethanol (0.550 g, 9.00 mmol) dropwise over two minutes, followed five minutes later by N,N-diisopropylethylamine (1.510 g, 2.10 mL, 11.7 mmol) dropwise over one minute. After stirring for 0.10 minutes at 23° C., the reaction was heated at 49° C. for three hours, then stirred at 23° C. for 18 hours. Acetic acid (4 mL) was added, and the reaction was heated at 80° C. for four hours. After cooling to 23° C., the mixture was concentrated. The residue was partitioned between ethyl acetate (250 mL) and saturated NaHCO₃ solution (60 mL) and separated. The organic layer was washed with saturated NaHCO₃ solution (30 mL), water (30 mL), and brine, then dried (Na₂SO₄), filtered, and concentrated. Silica gel chromatography (0 to 100% (10% isopropanol-90% ethyl acetate); 100 to 0% hexanes), followed by concentration and drying under vacuum, yielded the title compound (1.101 g, 65%) as an off-white solid.

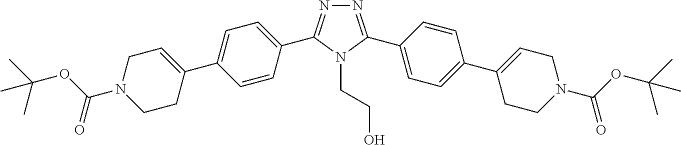 [SMILES: CC(C)(C)OC(=O)N1CC=C(c2ccc(-c3nnc(-c4ccc(C5=CCN(C(=O)OC(C)(C)C)CC5)cc4)n3CCO)cc2)CC1]

Synthesis of tert-butyl 4-{4-[5-(4-{1-[(tert-butoxy)carbonyl]-1,2,3,6-tetrahydropyridin-4-yl}phenyl)-4-(2-hydroxyethyl)-4H-1,2,4-triazol-3-yl]phenyl}-1,2,3,6-tetrahydropyridine-1-carboxylate: A mixture of 2-[3,5-bis-(4-bromo-phenyl)-[1,2,4]triazol-4-yl]-ethanol (0.100 g, 0.236 mmol), tert-butyl 5,6-dihydro-4-(4,4,5,5-tetramethyl-1,3,2-dioxaborolan-2-yl)pyridine-1(2H)-carboxylate (0.161 g, 0.520 mmol), 2 M Na₂CO₃ solution (0.591 mL, 1.18 mmol), and 1,1′-bis(diphenylphosphino)ferrocene]dichloropalladium(II), CH₂Cl₂ adduct (0.0193 g, 0.0236 mmol) in 1,4-dioxane (2.5 mL) was degassed by bubbling nitrogen through the mixture for several minutes. The reaction was heated at 80° C. for four hours. The mixture was partitioned between dichloromethane (90 mL) and saturated NaHCO₃ solution (20 mL) and separated. The aqueous layer was re-extracted with dichloromethane (2×20 mL). The organic layers were combined and washed with brine (15 mL), then dried (Na₂SO₄), filtered, and concentrated. Silica gel chromatography (0 to 100% (10% isopropanol-90% ethyl acetate); 100 to 0% hexanes), followed by concentration and drying under vacuum, yielded the title compound (0.1335 g, 90%) as a brown foam.

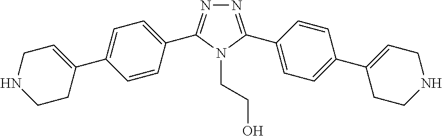 [SMILES: OCCn1c(-c2ccc(C3=CCNCC3)cc2)nnc1-c1ccc(C2=CCNCC2)cc1]

Synthesis of 2-{3,5-bis-[4-(1,2,3,6-tetrahydro-pyridin-4-yl)-phenyl]-[1,2,4]triazol-4-yl}-ethanol trifluoroacetate: To a solution of 4-{4-[5-(4-{1-[(tert-butoxy)carbonyl]-1,2,3,6-tetrahydropyridin-4-yl}phenyl)-4-(2-hydroxyethyl)-4H-1,2,4-triazol-3-yl]phenyl}-1,2,3,6-tetrahydropyridine-1-carboxylate (0.127 g, 0.202 mmol) in dichloromethane (2 mL) at 0° C. under N₂ was added trifluoroacetic acid (1.0 mL) dropwise. After five minutes, the ice bath was removed, and the reaction was stirred at 23° C. for two hours, then concentrated without heating. The residue was dissolved/suspended in water-acetonitrile, frozen at −78° C., then lyophilized to yield the title compound (0.1893 g, quantitative) as a brown solid. The material was used without purification in the next reaction.

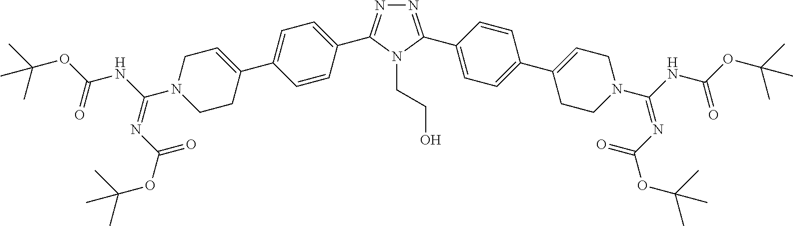 [SMILES: CC(C)(C)OC(=O)/N=C(/NC(=O)OC(C)(C)C)N1CC=C(c2ccc(-c3nnc(-c4ccc(C5=CCN(/C(=N\C(=O)OC(C)(C)C)NC(=O)OC(C)(C)C)CC5)cc4)n3CCO)cc2)CC1]

N—[(Z)-(4-{4-[5-(4-{1-[(Z)-{[(tert-butoxy)carbonyl]amino}({[(tert-butoxy)carbonyl]imino})methyl]-1,2,3,6-tetrahydropyridin-4-yl}phenyl)-4-(2-hydroxyethyl)-4H-1,2,4-triazol-3-yl]phenyl}-1,2,3,6-tetrahydropyridin-1-yl)({[(tert-butoxy)carbonyl]imino})methyl]carbamate: To a solution of 2-{3,5-bis-[4-(1,2,3,6-tetrahydro-pyridin-4-yl)-phenyl]-[1,2,4]triazol-4-yl}-ethanol trifluoroacetate (0.202 mmol) and N,N-diisopropylethylamine (0.183 g, 0.246 mL, 1.41 mmol) in N,N-dimethylformamide (2 mL) at 23° C. under N₂ was added tert-butyl N—[(Z)-{[(tert-butoxy)carbonyl]imino}(1H-pyrazol-1-yl)methyl]carbamate (0.188 g, 0.606 mmol). The reaction was stirred at 23° C. for 18 hours. 1-methylpiperazine (0.101 g, 0.112 mL, 1.01 mmol) was added, and the reaction was stirred for two hours. The mixture was partitioned between ethyl acetate (130 mL) and saturated NH₄Cl solution (20 mL) and separated. The organic layer was washed again with saturated NH₄Cl solution (15 mL), water (2×15 mL), saturated NaHCO₃ solution (15 mL), and brine (15 mL), then dried (Na₂SO₄), filtered, and concentrated. Silica gel chromatography (0 to 100% (15% isopropanol-85% ethyl acetate); 100 to 0% hexanes), followed by concentration and drying under vacuum, yielded a light yellow solid. The material was dissolved in EtOH (2 mL) at 23° C. under N₂, and 1 N NaOH solution (0.124 mL, 0.124 mmol) was added dropwise. Tetrahydrofuran (1 mL) was added and the reaction was stirred at 23° C. for 50 minutes, then heated to 40° C. for 90 minutes. After adding 6 N NaOH solution (0.062 mL), the reaction was heated to 40° C. for 45 minutes, then cooled to 23° C. Acetic acid (0.028 mL) was added dropwise, and the mixture was concentrated without heating. The mixture was partitioned between ethyl acetate (130 mL) and saturated NH₄Cl solution (20 mL) and separated. The organic layer was washed with saturated NaHCO₃ solution (30 mL), and brine (15 mL), then dried (Na₂SO₄), filtered, and concentrated. Silica gel chromatography (0 to 100% (15% isopropanol-85% ethyl acetate); 100 to 0% hexanes), followed by concentration and drying under vacuum, yielded the title compound (0.0711 g, 39%) as an off-white solid.

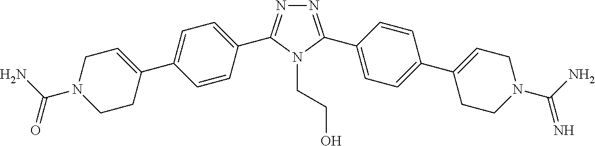 [SMILES: N=C(N)N1CC=C(c2ccc(-c3nnc(-c4ccc(C5=CCN(C(N)=O)CC5)cc4)n3CCO)cc2)CC1]

### Example 70

Synthesis of 4-(4-{5-[4-(1-carbamimidoyl-1,2,3,6-tetrahydropyridin-4-yl)phenyl]-4-(2-hydroxyethyl)-4H-1,2,4-triazol-3-yl}phenyl)-1,2,3,6-tetrahydropyridine-1-carboximidamide hydrochloride: To a solution of tert-butyl N—[(Z)-(4-{4-[5-(4-{1-[(Z)-{[(tert-butoxy) carbonyl]amino}({[(tert-butoxy)carbonyl]imino})methyl]-1,2,3,6-tetrahydropyridin-4-yl}phenyl)-4-(2-hydroxyethyl)-4H-1,2,4-triazol-3-yl]phenyl}-1,2,3,6-tetrahydropyridin-1-yl) ({[(tert-butoxy)carbonyl]imino})methyl]carbamate (0.0700 g, 0.0767 mmol) and anisole (0.0830 g, 0.0836 mL, 0.767 mmol) in dichloromethane (2.5 mL) at 0° C. under N₂ was added 4 N HCl-1,4-dioxane solution (0.500 mL, 2.00 mmol) dropwise. The ice bath was removed, and the mixture was sonicated occasionally to free the gummy precipitate from the walls of the vial. The reaction was stirred at 23° C. for three hours, then concentrated without heating. The residue was dissolved in HCl-EtOH solution (2 mL) and concentrated again, then suspended in ether. (2 mL) and stirred vigorously for ten minutes. The ether was removed via pipet, and the solid was dried under vacuum to yield the title compound (0.0500 g, quantitative) as a tan solid. ¹H NMR (300 MHz, DMSO-d₆) δ=7.81-7.75 (m, 4H), 7.72-7.66 (m, 4H), 7.58 (br s, 8H), 6.43-6.31 (m, 2H), 4.28-4.20 (m, 2H), 4.14-4.08 (m, J=3.4 Hz, 4H), 3.64 (br t, J=5.6 Hz, 4H), 3.25 (t, J=5.4 Hz, 2H), 2.67-2.59 (m, 4H). LC/MS (M+H)⁺=512.

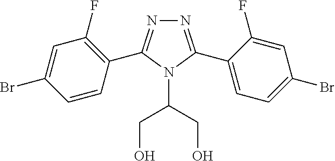 [SMILES: OCC(CO)n1c(-c2ccc(Br)cc2F)nnc1-c1ccc(Br)cc1F]

Synthesis of 2-[3,5-Bis-(4-bromo-2-fluoro-phenyl)-[1,2,4]triazol-4-yl]-propane-1,3-diol: To a slurry of (Z)-4-bromo-N—[(Z)-(4-bromo-2-fluorophenyl)(chloro)methylidene]-2-fluoro benzene-1-carbohydrazonoyl chloride (0.700 g, 1.49 mmol) in methanol (5 mL) at 0° C. under N₂ was added 2-amino-propane-1,3-diol (0.311 g, 3.42 mmol) dropwise, followed by dropwise addition of N,N-diisopropylethylamine (0.672 g, 0.906 mL, 5.20 mmol). After adding N,N-dimethylformamide (2 mL) for better solubility, the ice bath was removed, and the suspension was heated at 55° C. for 18 hours. The solution was cooled to 23° C., and acetic acid (2 mL) was added. The reaction was heated at 40° C. for eight hours, then stirred at 23° C. for 72 hours. The mixture was concentrated, then partitioned between ethyl acetate (250 mL) and saturated NaHCO₃ solution (60 mL) and separated. The organic layer was washed with saturated NaHCO₃ solution (25 mL), and brine (15 mL), then dried (Na₂SO₄), filtered, and concentrated. Silica gel chromatography (0 to 100% (10% isopropanol-90% ethyl acetate); 100 to 0% hexanes), followed by concentration and drying under vacuum, yielded the title compound which was used without further purification.

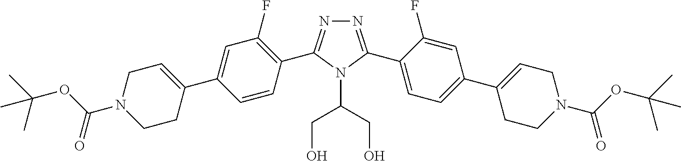 [SMILES: CC(C)(C)OC(=O)N1CC=C(c2ccc(-c3nnc(-c4ccc(C5=CCN(C(=O)OC(C)(C)C)CC5)cc4F)n3C(CO)CO)c(F)c2)CC1]

Synthesis of tert-butyl 4-{4-[5-(4-{1-[(tert-butoxy)carbonyl]-1,2,3,6-tetrahydropyridin-4-yl}-2-fluorophenyl)-4-(1,3-dihydroxypropan-2-yl)-4H-1,2,4-triazol-3-yl]-3-fluorophenyl}-1,2,3,6-tetrahydropyridine-1-carboxylate: A suspension of 2-[3,5-bis-(4-bromo-2-fluoro-phenyl)-[1,2,4]triazol-4-yl]-propane-1,3-diol (0.270 g, 0.552 mmol) in 1,4-dioxane (4 mL) was degassed by bubbling nitrogen through the mixture for several minutes. After adding tert-butyl 5,6-dihydro-4-(4,4,5,5-tetramethyl-1,3,2-dioxaborolan-2-yl)pyridine-1(2H)-carboxylate (0.376 g, 1.21 mmol), 2 M Na₂CO₃ solution (1.38 mL, 2.76 mmol), and 1,1′-bis(diphenylphosphino)ferrocene]dichloropalladium(II), CH₂Cl₂ adduct (0.0451 g, 0.0552 mmol), the reaction was degassed for two more minutes, then heated at 80° C. for two hours. The mixture was cooled to 23° C. and partitioned between 4:1 CHCl₃-isopropanol (80 mL) and saturated NaHCO₃ solution (20 mL) and separated. The aqueous layer was re-extracted with 4:1 CHCl₃-isopropanol (2×20 mL). The organic layers were combined and washed with brine (15 mL), then dried (Na₂SO₄), filtered, and concentrated. The residue was purified by reverse-phase chromatography (SiliaSep C₁₈ 40 g column; 5 to 100% MeCN-water (both with 0.1% trifluoroacetic acid) over 15 min.). The fractions containing the title compound were combined and partially concentrated (without heating) to remove most of the acetonitrile, then partitioned between 4:1 CHCl₃-isopropanol (80 mL) and saturated NaHCO₃ solution (35 mL) and separated. The aqueous layer was re-extracted with 4:1 CHCl₃-isopropanol (40 mL). The organic layers were combined and washed with brine (15 mL), then dried (Na₂SO₄), filtered, and concentrated. The compound was further purified by silica gel chromatography (0 to 100% (10% isopropanol-90% ethyl acetate); 100 to 0% hexanes), followed by concentration and drying under vacuum, to yield the title compound (0.0626 g, 6% over two steps) as a light brown solid.

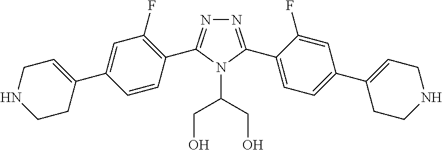 [SMILES: OCC(CO)n1c(-c2ccc(C3=CCNCC3)cc2F)nnc1-c1ccc(C2=CCNCC2)cc1F]

Synthesis of 2-{3,5-bis-[2-fluoro-4-(1,2,3,6-tetrahydro-pyridin-4-yl)-phenyl]-[1,2,4]triazol-4-yl}-propane-1,3-diol hydrochloride: To a suspension of tert-butyl 4-{4-[5-(4-{1-[(tert-butoxy)carbonyl]-1,2,3,6-tetrahydropyridin-4-yl}-2-fluorophenyl)-4-(1,3-dihydroxypropan-2-yl)-4H-1,2,4-triazol-3-yl]-3-fluorophenyl}-1,2,3,6-tetrahydropyridine-1-carboxylate (0.0600 g, 0.0865 mmol) and anisole (0.0830 g, 0.0836 mL, 0.767 mmol) in dichloromethane (2.5 mL) at 23° C. under N₂ was added 4 N HCl-1,4-dioxane solution (0.500 mL, 2.00 mmol) dropwise. After three hours, the reaction was concentrated. The residue was suspended in CH₂Cl₂ (2 mL) and concentrated again, then dried under vacuum to yield the title compound (0.0935 g, quantitative) as a tan solid.

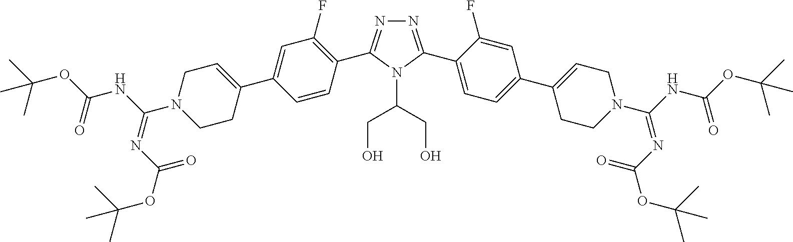 [SMILES: CC(C)(C)OC(=O)/N=C(/NC(=O)OC(C)(C)C)N1CC=C(c2ccc(-c3nnc(-c4ccc(C5=CCN(/C(=N\C(=O)OC(C)(C)C)NC(=O)OC(C)(C)C)CC5)cc4F)n3C(CO)CO)c(F)c2)CC1]

Synthesis of tert-butyl N—[(Z)-(4-{4-[5-(4-{1-[(Z)-{[(tert-butoxy)carbonyl]amino}({[(tert-butoxy)carbonyl]imino})methyl]-1,2,3,6-tetrahydropyridin-4-yl}-2-fluorophenyl)-4-(1,3-dihydroxypropan-2-yl)-4H-1,2,4-triazol-3-yl]-3-fluorophenyl}-1,2,3,6-tetrahydro pyridin-1-yl)({[(tert-butoxy)carbonyl]imino})methyl]carbamate: To a suspension of 2-{3,5-bis-[2-fluoro-4-(1,2,3,6-tetrahydro-pyridin-4-yl)-phenyl]-[1,2,4]triazol-4-yl}-propane-1,3-diol hydrochloride (0.0516 g, 0.0856 mmol) and N,N-diisopropylethylamine (0.0774 g, 0.104 mL, 0.599 mmol) in N,N-dimethylformamide (2 mL) at 23° C. under N₂ was added tert-butyl. N—[(Z)-{[(tert-butoxy)carbonyl]imino}(1H-pyrazol-1-yl)methyl]carbamate (0.0797 g, 0.257 mmol). The reaction was stirred at 23° C. for 18 hours. LC-MS analysis of the suspension showed unreacted amine. After adding additional N, N-dimethylformamide (2 mL), the mixture was heated to 40° C. for 18 hours. The reaction was cooled to 23° C. In order to scavenge unreacted guanylating reagent, 1-methylpiperazine (0.0429 g, 0.0475 mL, 0.428 mmol) was added, and the reaction was stirred for two hours. The mixture was partitioned between 4:1 CHCl₃ isopropanol. (80 mL) and saturated NH₄Cl solution (20 mL) and separated. The aqueous layer was re-extracted with 4:1 CHCl₃-isopropanol (2×20 mL). The organic layers were combined and washed with saturated NH₄Cl solution (15 mL), saturated NaHCO₃ solution (15 mL), and brine (15 mL), then dried (Na₂SO₄), filtered, and concentrated. Silica gel chromatography (0 to 100% (15% isopropanol-85% ethyl acetate); 100 to 0% hexanes), followed by concentration and drying under vacuum, yielded the title compound (0.0449 g, 54%) as an off-white solid.

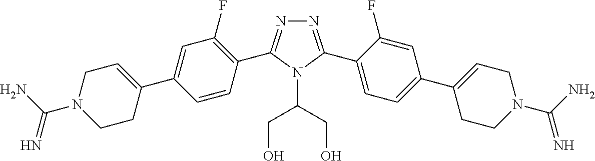 [SMILES: N=C(N)N1CC=C(c2ccc(-c3nnc(-c4ccc(C5=CCN(C(=N)N)CC5)cc4F)n3C(CO)CO)c(F)c2)CC1]

### Example 71

Synthesis of 4-(4-{5-[4-(1-carbamimidoyl-1,2,3,6-tetrahydropyridin-4-yl)-2-fluorophenyl]-4-(1,3-dihydroxypropan-2-yl}-4H-1,2,4-triazol-3-yl)-3-fluorophenyl)-1,2,3,6-tetrahydropyridine-1-carboximidamide hydrochloride: To a solution of tert-butyl N—[(Z)-(4-{4-[5-(4-{1-[(Z)-{[(tert-butoxy)carbonyl]amino}({[(tert-butoxy)carbonyl]imino})methyl]-1,2,3,6-tetrahydropyridin-4-yl}-2-fluorophenyl)-4-(1,3-dihydroxypropan-2-yl)-4H-1,2,4-triazol-3-yl]-3-fluorophenyl}-1,2,3,6-tetrahydropyridin-1-yl)({[(tert-butoxy)carbonyl]imino})methyl]carbamate (0.0449 g, 0.0459 mmol) and anisole (0.0496 g, 0.0500 mL, 0.459 mmol) in dichloromethane (2.5 mL) at 23° C. under N₂ was added 4 N HCl-dioxane solution (0.600 mL, 2.40 mmol) dropwise. After three hours, the reaction was concentrated. The residue was suspended in CH₂Cl₂ and concentrated again (2×). The residue was suspended in ethyl acetate (2.5 mL) and stirred vigorously for 15 minutes, then centrifuged for several minutes to spin the solid to the bottom of the vial. The ethyl acetate was withdrawn with a pipet, and the trituration was repeated. Drying under vacuum yielded the title compound (0.0445 g, 97%) as an off-white solid. ¹H NMR (300 MHz, DMSO-d₆) δ=7.65-7.47 (m, 14H), 6.51-6.41 (m, 2H), 4.15-4.06 (m, J=3.5 Hz, 4H), 4.00 (br tdd, J=7.1, 7.2, 7.3 Hz, 1H), 3.63 (br t, J=5.3 Hz, 4H), 3.35 (br d, J=7.2 Hz, 4H), 2.67-2.58 (m, 4H). ¹⁹F NMR (282 MHz, DMSO-d₆) δ=−112.05-−112.14 (m, 1F). LC/MS (M+H)⁺=578.

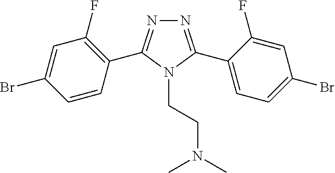 [SMILES: CN(C)CCn1c(-c2ccc(Br)cc2F)nnc1-c1ccc(Br)cc1F]

Synthesis of {2-[3,5-bis-(4-bromo-2-fluoro-phenyl)-[1,2,4]triazol-4-yl]-ethyl}-dimethyl-amine: To a suspension of (Z)-4-bromo-N—[(Z)-(4-bromo-2-fluorophenyl)(chloro) methylidene]-2-fluorobenzene-1-carbohydrazonoyl chloride (0.570 g, 1.21 mmol) in methanol (5 mL) at 0° C. under N₂ was added (2-aminoethyl)dimethylamine (0.128 g, 0.159 mL, 1.45 mmol), followed by N,N-diisopropylethylamine (0.469 g, 0.633 mL, 3.63 mmol). The ice bath was removed, and the suspension was heated at 60° C. for 18 hours. The reaction was cooled to 23° C. before adding acetic acid (2 mL), heating at 60° C. for seven hours, then stirring at 23° C. for 18 hours. The mixture was concentrated, then partitioned between ethyl acetate (250 mL) and saturated NaHCO₃ solution (50 mL) and separated. The organic layer was washed with saturated NaHCO₃ solution (25 mL), and brine (25 mL), then dried (Na₂SO₄), filtered, and concentrated. Silica gel chromatography (0 to 100% (15% isopropanol-85% ethyl acetate); 100 to 0% hexanes), followed by concentration and drying under vacuum, yielded the title compound (0.3242 g, 55%) as an off-white solid.

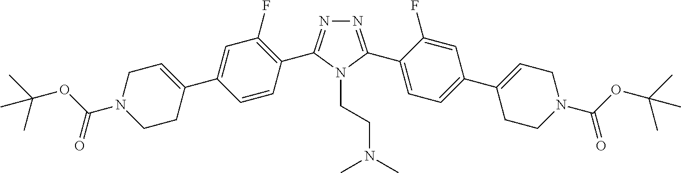 [SMILES: CN(C)CCn1c(-c2ccc(C3=CCN(C(=O)OC(C)(C)C)CC3)cc2F)nnc1-c1ccc(C2=CCN(C(=O)OC(C)(C)C)CC2)cc1F]

Synthesis of tert-butyl 4-{4-[5-(4-{1-[(tert-butoxy)carbonyl]-1,2,3,6-tetrahydropyridin-4-yl}-2-fluorophenyl)-4-[2-(dimethylamino)ethyl]-4H-1,2,4-triazol-3-yl]-3-fluorophenyl}-1,2,3,6-tetrahydropyridine-1-carboxylate: A solution of tert-butyl 5,6-dihydro-4-(4,4,5,5-tetramethyl-1,3,2-dioxaborolan-2-yl)pyridine-1(2H)-carboxylate (0.175 g, 0.566 mmol) in 1,4-dioxane (2.5 mL) was degassed by bubbling nitrogen through the mixture for several minutes. After adding 2 M Na₂CO₃ solution (0.643 mL, 1.29 mmol), {2-[3,5-bis-(4-bromo-2-fluoro-phenyl)-[1,2,4]triazol-4-yl]-ethyl}-dimethyl-amine (0.125 g, 0.257 mmol), and 1,1′-bis(diphenylphosphino)ferrocene]dichloro palladium(II), CH₂Cl₂ adduct (0.0210 g, 0.0257 mmol), the reaction was degassed for two more minutes, then heated at 80° C. for 2.5 hours. The mixture was cooled to 23° C. and partitioned between 4:1 CHCl₃-isopropanol (80 mL) and saturated NaHCO₃ solution (20 mL) and separated. The aqueous layer was re-extracted with 4:1 CHCl₃-isopropanol (2×20 mL). The organic layers were combined and washed with brine (15 mL), then dried (Na₂SO₄), filtered, and concentrated. Silica gel chromatography (0 to 100% (25% isopropanol-75% ethyl acetate); 100 to 0% hexanes), followed by concentration and drying under vacuum, yielded the title compound (0.1341 g, 76%) as a brown foam.

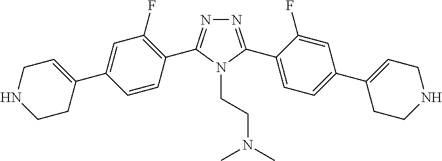 [SMILES: CN(C)CCn1c(-c2ccc(C3=CCNCC3)cc2F)nnc1-c1ccc(C2=CCNCC2)cc1F]

Synthesis of (2-{3,5-bis-[2-fluoro-4-(1,2,3,6-tetrahydro-pyridin-4-yl)-phenyl]-[1,2,4]triazol-4-yl}-ethyl)-dimethyl-amine hydrochloride: To a suspension of tert-butyl 4-{4-[5-(4-{1-[(tert-butoxy)carbonyl]-1,2,3,6-tetrahydropyridin-4-yl}-2-fluorophenyl)-4-[2-(dimethylamino)ethyl]-4H-1,2,4-triazol-3-yl]-3-fluorophenyl}-1,2,3,6-tetrahydropyridine-1-carboxylate (0.132 g, 0.191 mmol) and anisole (0.207 g, 0.208 mL, 1.91 mmol) in dichloromethane (2.5 mL) at 23° C. under N₂ was added 4 N HCl-1,4-dioxane solution (0.700 mL, 2.80 mmol) dropwise. A gummy precipitate began to form—the vial was occasionally sonicated to keep it suspended. After 2.5 hours, an additional 0.300 mL of 4 N HCl-1,4-dioxane was added, and the reaction was stirred for 30 minutes. The mixture was concentrated. The residue was suspended in CH₂Cl₂ (2 mL) and concentrated again (2×), then dried under vacuum to yield the title compound (0.1276 g, quantitative) as a tan solid

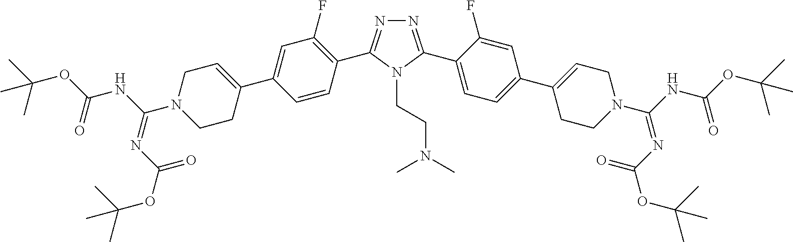 [SMILES: CN(C)CCn1c(-c2ccc(C3=CCN(/C(=N\C(=O)OC(C)(C)C)NC(=O)OC(C)(C)C)CC3)cc2F)nnc1-c1ccc(C2=CCN(/C(=N\C(=O)OC(C)(C)C)NC(=O)OC(C)(C)C)CC2)cc1F]

Synthesis of tert-butyl N—[(Z)-(4-{4-[5-(4-{1-[(Z)-{[(tert-butoxy)carbonyl]amino}({[(tert-butoxy)carbonyl]imino})methyl]-1,2,3,6-tetrahydropyridin-4-yl}-2-fluorophenyl)-4-[2-(dimethylamino)ethyl]-4H-1,2,4-triazol-3-yl]-3-fluorophenyl}-1,2,3,6-tetrahydropyridin-1-yl)({[(tert-butoxy)carbonyl]imino})methyl]carbamate: To a solution of (2-{3,5-bis-[2-fluoro-4-(1,2,3,6-tetrahydro-pyridin-4-yl)-phenyl]-[1,2,4]triazol-4-yl}-ethyl)-dimethyl-amine hydrochloride (0.191 mmol) and N,N-diisopropylethylamine (0.173 g, 0.233 mL, 1.34 mmol) in N,N-dimethylformamide (2 mL) at 23° C. under N₂ was added tert-butyl N—[(Z)-{[(tert-butoxy)carbonyl]imino}(1H-pyrazol-1-yl)methyl]carbamate (0.178 g, 0.573 mmol). The reaction was stirred at 23° C. for 72 hours. 1-methylpiperazine (0.0957 g, 0.106 mL, 0.955 mmol) was added, and the reaction was stirred for two hours. The mixture was partitioned between ethyl acetate (130 mL) and saturated NH₄Cl solution (20 mL) and separated. The organic layer was washed again with saturated NH₄Cl solution (15 mL), water (2×1.5 mL), saturated NaHCO₃ solution (15 mL), and brine (15 mL), then dried (Na₂SO₄), filtered, and concentrated. The residue was purified by reverse-phase chromatography (SiliaSep C₁₈ 40 g column; 5 to 100% MeCN-water (both with 0.1% trifluoroacetic acid) over 22 min.). The fractions containing the title compound were combined and partially concentrated (without heating) to remove most of the acetonitrile, then partitioned between 4:1 CHCl₃-isopropanol (80 mL) and saturated NaHCO₃ solution (35 mL) and separated. The aqueous layer was re-extracted with 4:1 CHCl₃-isopropanol (40 mL). The organic layers were combined and washed with brine (15 mL), then dried (Na₂SO₄), filtered, and concentrated. The compound was further purified by silica gel chromatography (0 to 100% (33% isopropanol-67% ethyl acetate); 100 to 0% hexanes), followed by concentration and drying under vacuum, to yield the title compound (0.1182 g, 63%) as an off-white solid.

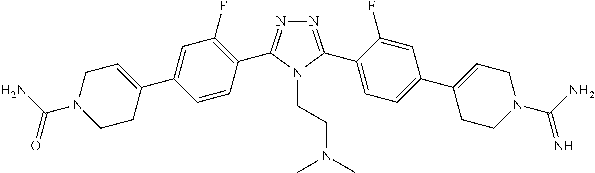 [SMILES: CN(C)CCn1c(-c2ccc(C3=CCN(C(=N)N)CC3)cc2F)nnc1-c1ccc(C2=CCN(C(N)=O)CC2)cc1F]

### Example 72

Synthesis of 4-(4-{5-[4-(1-carbamimidoyl-1,2,3,6-tetrahydropyridin-4-yl)-2-fluorophenyl]-4-[2-(dimethylamino)ethyl]-4H-1,2,4-triazol-3-yl}-3-fluorophenyl)-1,2,3,6-tetrahydropyridine-1-carboximidamide hydrochloride: To a solution of tert-butyl N—[(Z)-(4-{4-[5-(4-{1-[(Z)-{[(tert-butoxy)carbonyl]amino}({[(tert-butoxy)carbonyl]imino})methyl]-1,2,3,6-tetrahydropyridin-4-yl}-2-fluorophenyl)-4-[2-(dimethylamino)ethyl]-4H-1,2,4-triazol-3-yl]-3-fluorophenyl}-1,2,3,6-tetrahydropyridin-1-yl)({[(tert-butoxy)carbonyl]imino})methyl]carbamate (0.1.16 g, 0.1.19 mmol) and anisole (0.129 g, 0.130 mL, 1.19 mmol) in dichloromethane (3 mL) in a bath of cool tap water under N₂ was added 4 N HCl-1,4-dioxane solution (0.750 mL, 3.00 mmol) dropwise. The cooling bath was removed, and after three hours, the reaction was concentrated. The residue was suspended in CH₂Cl₂ and concentrated again (2×). The residue was suspended/dissolved in water −acetonitrile, frozen at −78° C., and lyophilized to yield the title compound (0.0690 g, 81%) as a flocculent, off-white solid. ¹H NMR (300 MHz, DMSO-d₆) δ=11.05 (br s, 1H), 7.76-7.69 (m, 2H), 7.68-7.51 (m, 12H), 6.55-6.43 (m, 2H), 4.45-4.21 (m, 2H), 4.19-4.07 (m, J=3.4 Hz, 4H), 3.65 (br t, J=5.5 Hz, 4H), 3.00-2.82 (m, 2H), 2.68-2.58 (m, 4H), 2.44 (br d, J=5.9 Hz, 6H). ¹⁹F NMR (282 MHz, DMSO-d₆) δ=−113.15-−113.29 (m, 1F). LC/MS (M+H)⁺=575.

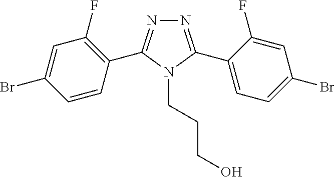 [SMILES: OCCCn1c(-c2ccc(Br)cc2F)nnc1-c1ccc(Br)cc1F]

dbw5-114-b:

Synthesis of 3-[3,5-bis-(4-bromo-2-fluoro-phenyl)-[1,2,4]triazol-4-yl]-propan-1-ol: To a suspension of (Z)-4-bromo-N—[(Z)-(4-bromo-2-fluorophenyl)(chloro)methylidene]-2-fluorobenzene-1-carbohydrazonoyl chloride (0.600 g, 1.27 mmol) in anhydrous toluene (6 mL) at 0° C. under N₂ was added 3-amino-propan-1-ol (0.383 g, 0.390 mL, 5.10 mmol) dropwise. The ice bath was removed, and the mixture was sonicated briefly, then stirred at 23° C. for 72 hours. The reaction was heated to 100° C. for three hours, then cooled to 23° C. Acetic acid (1 mL) was added; the mixture was heated to 60° C. for 90 minutes, then stirred at 23° C. for 18 hours. The mixture was partitioned between ethyl acetate (250 mL) and saturated NaHCO₃ solution (50 mL) and separated. The organic layer was washed with saturated NaHCO₃ solution (25 mL), and brine (25 mL), then dried (Na₂SO₄), filtered, and concentrated. Silica gel chromatography (0 to 100% (10% isopropanol-90% ethyl acetate); 0.100 to 0% hexanes), followed by concentration and drying under vacuum, yielded the title compound (0.3418 g, 57%) as an off-white solid.

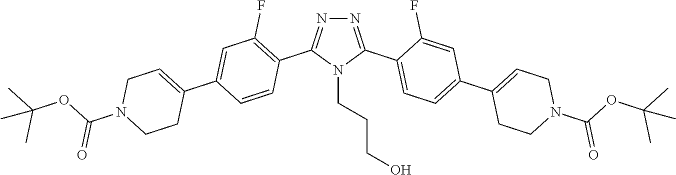 [SMILES: CC(C)(C)OC(=O)N1CC=C(c2ccc(-c3nnc(-c4ccc(C5=CCN(C(=O)OC(C)(C)C)CC5)cc4F)n3CCCO)c(F)c2)CC1]

Synthesis of tert-butyl 4-{4-[5-(4-{1-[(tert-butoxy)carbonyl]-1,2,3,6-tetrahydropyridin-4-yl}-2-fluorophenyl)-4-(3-hydroxypropyl)-4H-1,2,4-triazol-3-yl]-3-fluorophenyl}-1,2,3,6-tetrahydropyridine-1-carboxylate: A solution of tert-butyl 5,6-dihydro-4-(4,4,5,5-tetramethyl-1,3,2-dioxaborolan-2-yl)pyridine-1(2H)-carboxylate (0.180 g, 0.581 mmol) in N,N-dimethylfomamide (2.5 mL) was degassed by bubbling nitrogen through the mixture for several minutes. After adding 2 M Na₂CO₃ solution (0.661 mL, 1.32 mmol), 3-[3,5-bis-(4-bromo-2-fluoro-phenyl)-[1,2,4]triazol-4-yl]-propan-1-ol (0.125 g, 0.264 mmol), and 1,1′-bis(diphenylphosphino)ferrocene]dichloro palladium(II), CH₂Cl₂ adduct (0.0216 g, 0.0264 mmol), the reaction was degassed for two more minutes, then heated at 80° C. for two hours. The mixture was cooled to 23° C. and partitioned between 4:1 CHCl₃-isopropanol (80 mL) and saturated NaHCO₃ solution (20 mL) and separated. The aqueous layer was re-extracted with 4:1 CHCl₃-isopropanol (2×20 mL). The organic layers were combined and washed with brine (15 mL), then dried (Na₂SO₄), filtered, and concentrated. Silica gel chromatography (0 to 100% (10% isopropanol 90% ethyl acetate); 100 to 0% CH₂Cl₂), followed by concentration and drying under vacuum, yielded a brown foam. This material was further purified by reverse-phase chromatography (SiliaSep C₁₈ 40 g column; 5 to 100% MeCN-water (both with 0.1% trifluoroacetic acid) over 22 min.). The fractions containing the title compound were combined and partially concentrated (without heating) to remove the acetonitrile, then partitioned between 4:1 CHCl₃-isopropanol (80 mL) and saturated NaHCO₃ solution (35 mL) and separated. The aqueous layer was re-extracted with 4:1 CHCl₃-isopropanol (2×20 mL). The organic layers were combined and washed with brine (15 mL), then dried (Na₂SO₄), filtered, and concentrated to yield the title compound (0.11.93 g, 67%) as an off-white solid.

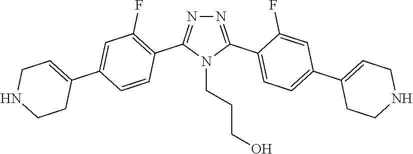 [SMILES: OCCCn1c(-c2ccc(C3=CCNCC3)cc2F)nnc1-c1ccc(C2=CCNCC2)cc1F]

Synthesis of 3-{3,5-bis-[2-fluoro-4-(1,2,3,6-tetrahydro-pyridin-4-yl)-phenyl]-[1,2,4]triazol-4-yl}-propan-1-ol hydrochloride: To a solution of tert-butyl 4-{4-[5-(4-{1-[(tert-butoxy) carbonyl]-1,2,3,6-tetrahydropyridin-4-yl}-2-fluorophenyl)-4-(3-hydroxypropyl)-4H-1,2,4-triazol-3-yl]-3-fluorophenyl}-1,2,3,6-tetrahydropyridine-1-carboxylate (0.117 g, 0.173 mmol) and anisole (0.187 g, 0.188 mL, 1.73 mmol) in dichloromethane (3 mL) at 23° C. under N₂ was added 4 N HCl-1,4-dioxane solution (1.00 mL, 4.00 mmol) dropwise. The vial was occasionally sonicated to keep it suspended. After four hours, the mixture was concentrated. The residue was suspended in CH₂Cl₂ (2 mL) and concentrated again (2×), then dissolved in MeCN-water and lyophilized to yield the title compound (0.0971 g, 96%) as a flocculent, off-white solid.

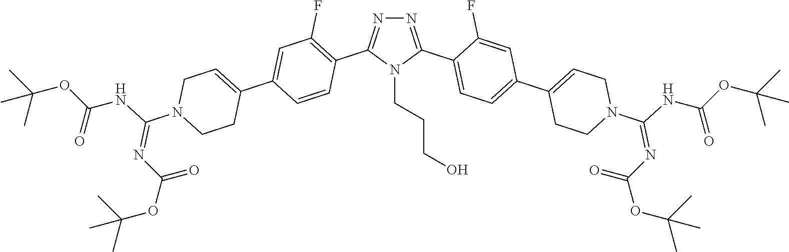

dbw5-129-b:

Synthesis of tert-butyl N—[(Z)-(4-{4-[5-(4-{1-[(Z)-{[(tert-butoxy) carbonyl]amino}({[(tert-butoxy)carbonyl]imino})methyl]-1,2,3,6-tetrahydropyridin-4-yl}-2-fluorophenyl)-4-(3-hydroxypropyl)-4H-1,2,4-triazol-3-yl]-3-fluorophenyl}-1,2,3,6-tetrahydropyridin-1-yl)({[(tert-butoxy)carbonyl]imino})methyl]carbamate: To a suspension of 3-{3,5-bis-[2-fluoro-4-(1,2,3,6-tetrahydro-pyridin-4-yl)-phenyl]-[1,2,4]triazol-4-yl}-propan-1-ol hydrochloride (0.0917 g, 0.156 mmol) and N,N-diisopropylethylamine (0.141 g, 0.191 mL, 1.09 mmol) in N,N-dimethylformamide (3 mL) at 23° C. under N₂ was added tert-butyl. N—[(Z)-{[(tert-butoxy)carbonyl]imino}(1H-pyrazol-1-yl)methyl]carbamate (0.145 g, 0.469 mmol). The reaction was stirred at 23° C. for 72 hours. The homogeneous mixture was partitioned between ethyl acetate (130 mL) and saturated NH₄Cl solution (35 mL) and separated. The organic layer was washed with water (2×15 mL), saturated NaHCO₃ solution (15 mL), and brine (15 mL), then dried (Na₂SO₄), filtered, and concentrated. Silica gel chromatography (0 to 100% (15% isopropanol 85% ethyl acetate); 100 to 0% hexanes), followed by concentration and drying under vacuum, yielded the title compound (0.1131 g, 75%) as a white solid.

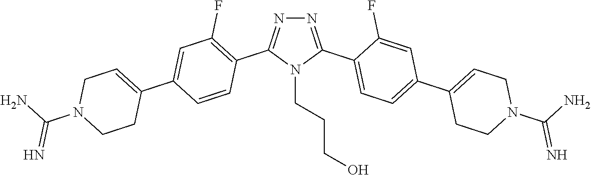 [SMILES: N=C(N)N1CC=C(c2ccc(-c3nnc(-c4ccc(C5=CCN(C(=N)N)CC5)cc4F)n3CCCO)c(F)c2)CC1]

### Example 73

Synthesis of 4-(4-{5-[4-(1-carbamimidoyl-1,2,3,6-tetrahydropyridin-4-yl)-2-fluorophenyl]-4-(3-hydroxypropyl)-4H-1,2,4-triazol-3-yl}-3-fluorophenyl)-1,2,3,6-tetrahydropyridine-1-carboximidamide hydrochloride: To a solution of tert-butyl N—[(Z)-(4-{4-[5-(4-{1-[(Z)-{[(tert-butoxy)carbonyl]amino}({[(tert-butoxy)carbonyl]imino})methyl]-1,2,3,6-tetrahydropyridin-4-yl}-2-fluorophenyl)-4-(3-hydroxypropyl)-4H-1,2,4-triazol-3-yl]-3-fluorophenyl}-1,2,3,6-tetrahydropyridin-1-yl)({[(tert-butoxy)carbonyl]imino})methyl]carbamate (0.111 g, 0.115 mmol) and anisole (0.125 g, 0.126 mL, 1.1.5 mmol) in dichloromethane (3 mL) at 23° C. under N₂ was added 4 N HCl-1,4-dioxane solution (1.00 mL, 4.00 mmol) dropwise. The vial was occasionally sonicated to keep it suspended. After 3.5 hours, the mixture was concentrated. The residue was dissolved in 4:1 MeCN-water and lyophilized to yield the title compound (0.0751 g, 97%) as a flocculent, off-white solid. ¹H NMR. (300 MHz, DMSO-d₆) δ=7.70-7.49 (m, 14H), 6.55-6.40 (m, 2H), 4.15-4.07 (m, J=2.9 Hz, 4H), 3.95-3.87 (m, 4H), 3.63 (br t, J==5.3 Hz, 4H), 3.02 (t, J=5.9 Hz, 2H), 2.68-2.57 (m, 4H), 1.42-1.27 (m, 2H). LC/MS (M+H)+=562.

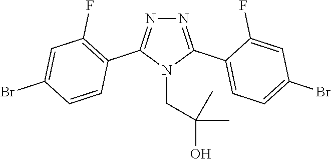 [SMILES: CC(C)(O)Cn1c(-c2ccc(Br)cc2F)nnc1-c1ccc(Br)cc1F]

Synthesis of 1-[3,5-bis-(4-bromo-2-fluoro-phenyl)-[1,2,4]triazol-4-yl]-2-methyl-propan-2-ol: To a solution of (Z)-4-bromo-N—[(Z)-(4-bromo-2-fluorophenyl)(chloro)methylidene]-2-fluorobenzene-1-carbohydrazonoyl chloride (0.500 g, 1.06 mmol) in anhydrous toluene (6 mL) at 0° C. under N₂ was added 1-amino-2-methyl-propan-2-ol (0.379 g, 0.396 mL, 4.25 mmol) dropwise. After 60 minutes, the reaction was permitted to warm to 23° C. for 18 hours. Acetic acid (1 mL) was added; the mixture was heated to 60° C. for two hours, then concentrated. The mixture was partitioned between ethyl acetate (130 mL) and saturated NaHCO₃ solution (30 mL) and separated. The organic layer was washed with saturated NaHCO₃ solution (15 mL), and brine (15 mL), then dried (Na₂SO₄), filtered, and concentrated. The residue was purified by reverse-phase chromatography (SiliaSep C₁₈ 40 g column; 5 to 100% MeCN-water (both with 0.1% trifluoroacetic acid) over 22 min.). The fractions containing the title compound were combined and partially concentrated (without heating) to remove most of the acetonitrile, then partitioned between 4:1 CHCl₃-isopropanol (80 mL) and saturated NaHCO₃ solution (35 mL) and separated. The aqueous layer was re-extracted with 4:1 CHCl₃-isopropanol. (2×20 mL). The organic layers were combined and washed with brine (15 mL), then dried (Na₂SO₄), filtered, and concentrated to yield the title compound (0.0798 g, 16%) as an off-white solid.

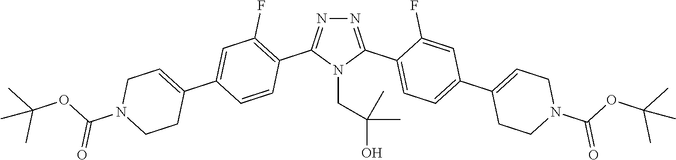 [SMILES: CC(C)(O)Cn1c(-c2ccc(C3=CCN(C(=O)OC(C)(C)C)CC3)cc2F)nnc1-c1ccc(C2=CCN(C(=O)OC(C)(C)C)CC2)cc1F]

Synthesis of 4-{4-[5-(4-{1-[(tert-butoxy)carbonyl]-1,2,3,6-tetrahydropyridin-4-yl}-2-fluorophenyl)-4-(2-hydroxy-2-methylpropyl)-4H-1,2,4-triazol-3-yl]-3-fluorophenyl}-1,2,3,6-tetrahydropyridine-1-carboxylate: A solution of tert-butyl 5,6-dihydro-4-(4,4,5,5-tetramethyl-1,3,2-dioxaborolan-2-yl)pyridine-1(2H)-carboxylate (0.108 g, 0.348 mmol) in 1,4-dioxane (2.5 mL) was degassed by bubbling nitrogen through the mixture for several minutes. After adding 2 M Na₂CO₃ solution (0.395 mL, 0.790 mmol), 1-[3,5-bis-(4-bromo-2-fluoro-phenyl)-[1,2,4]triazol-4-yl]-2-methyl-propan-2-ol (0.0770 g, 0.158 mmol), and 1,1′-bis(diphenylphosphino)ferrocene]dichloro palladium(II), CH₂Cl₂ adduct (0.0129 g, 0.0158 mmol), the reaction was degassed for two more minutes, then heated at 80° C. for 2.5 hours and stirred at 23° C. for 18 hours. The mixture was partitioned between CH₂Cl₂ (80 mL) and saturated NaHCO₃ solution (20 mL) and separated. The aqueous layer was re-extracted with CH₂Cl₂ (2×20 mL). The organic layers were combined and washed with brine (15 mL), then dried (Na₂SO₄), filtered, and concentrated. Silica gel chromatography (0 to 100% (15% isopropanol-85% ethyl acetate); 100 to 0% hexanes), followed by concentration and drying under vacuum, yielded the title compound (0.0939 g, 86%) as a tan solid.

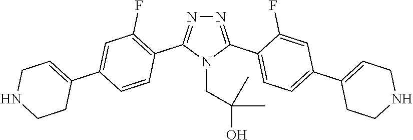 [SMILES: CC(C)(O)Cn1c(-c2ccc(C3=CCNCC3)cc2F)nnc1-c1ccc(C2=CCNCC2)cc1F]

Synthesis of 1-{3,5-bis-[2-fluoro-4-(1,2,3,6-tetrahydro-pyridin-4-yl)-phenyl]-[1,2,4]triazol-4-yl}-2-methyl-propan-2-ol hydrochloride: To a solution of tert-butyl 4-{4-[5-(4-{1-[(tert-butoxy)carbonyl]-1,2,3,6-tetrahydropyridin-4-yl}-2-fluorophenyl)-4-(2-hydroxy-2-methylpropyl)-4H-1,2,4-triazol-3-yl]-3-fluorophenyl}-1,2,3,6-tetrahydropyridine-1-carboxylate (0.0912 g, 0.132 mmol) and anisole (0.143 g, 0.144 mL, 1.32 mmol) in dichloromethane (3 mL) at 23° C. under. N₂ was added 4 N HCl-1,4-dioxane solution (1.00 mL, 4.00 mmol) dropwise. The vial was occasionally sonicated to keep it suspended. After 2.5 hours, the mixture was concentrated, then suspended in CH₂Cl₂ and concentrated again (2×). The residue was dissolved in 4:1 MeCN-water and lyophilized to yield the title compound (0.0812 g, quantitative) as a flocculent, off-white solid.

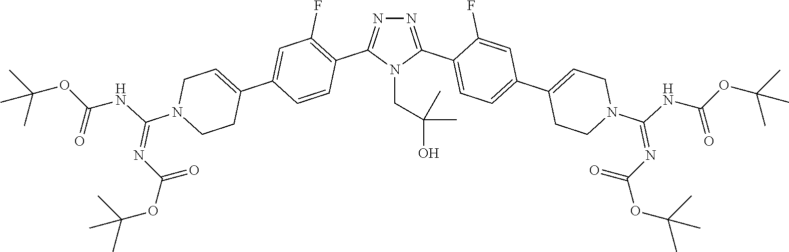

Synthesis of tert-butyl N—[(Z)-(4-{4-[5-(4-{1-[(Z)-{[(tert-butoxy)carbonyl]amino}({[(tert-butoxy)carbonyl]imino})methyl]-1,2,3,6-tetrahydropyridin-4-yl}-2-fluorophenyl)-4-(2-hydroxy-2-methylpropyl)-4H-1,2,4-triazol-3-yl]-3-fluorophenyl}-1,2,3,6-tetrahydro pyridin-1-yl)({[(tert-butoxy)carbonyl]imino})methyl]carbamate: To a suspension of 1-{3,5-bis-[2-fluoro-4-(1,2,3,6-tetrahydro-pyridin-4-yl)-phenyl]-[1,2,4]triazol-4-yl}-2-methyl-propan-2-ol hydrochloride (0.0756 g, 0.126 mmol) and N,N-diisopropylethylamine (0.114 g, 0.153 mL, 0.881 mmol) in N,N-dimethylformamide (2 mL) at 23° C. under N₂ was added tert-butyl N—[(Z)-{[(tert-butoxy)carbonyl] imino}(1H-pyrazol-1-yl)methyl]carbamate (0.117 g, 0.377 mmol). The reaction was stirred at 23° C. for 72 hours. The homogeneous mixture was partitioned between ethyl acetate (130 mL) and saturated NH₄Cl solution (15 mL) and separated. The organic layer was washed with water (2×15 mL), saturated NaHCO₃ solution (15 mL), and brine (15 mL), then dried (Na₂SO₄), filtered, and concentrated. Silica gel chromatography (0 to 100% (15% isopropanol-85% ethyl acetate); 100 to 0% hexanes), followed by concentration and drying under vacuum, yielded the title compound (0.0911 g, 72%) as a tan solid.

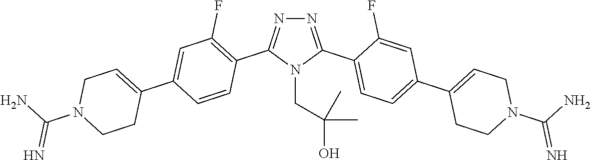 [SMILES: CC(C)(O)Cn1c(-c2ccc(C3=CCN(C(=N)N)CC3)cc2F)nnc1-c1ccc(C2=CCN(C(=N)N)CC2)cc1F]

### Example 74

Synthesis of 4-(4-{5-[4-(1-carbamimidoyl-1,2,3,6-tetrahydropyridin-4-yl)-2-fluorophenyl]-4-(2-hydroxy-2-methylpropyl)-4H-1,2,4-triazol-3-yl}-3-fluorophenyl)-1,2,3,6-tetrahydropyridine-1-carboximidamide hydrochloride: To a solution of tert-butyl N—[(Z)-(4-{4-[5-(4-{1-[(Z)-{[(tert-butoxy)carbonyl]amino}({[(tert-butoxy)carbonyl]imino})methyl]-1,2,3,6-tetrahydropyridin-4-yl}-2-fluorophenyl)-4-(2-hydroxy-2-methylpropyl)-4H-1,2,4-triazol-3-yl]-3-fluorophenyl}-1,2,3,6-tetrahydropyridin-1-yl)({[(tert-butoxy)carbonyl]imino})methyl]carbamate (0.0870 g, 0.0891 mmol) and anisole (0.0964 g, 0.0971 mL, 0.891 mmol) in dichloromethane (3 mL) at 23° C. under N₂ was added 4 N HCl-1,4-dioxane solution (1.00 mL, 4.00 mmol) dropwise. The vial was occasionally sonicated to keep it suspended. After three hours, the mixture was concentrated, then suspended in CH₂Cl₂ and concentrated again (2×). The residue was dissolved in 4:1 MeCN-water, frozen at −78° C., then lyophilized to yield the title compound (0.0593 g, 97%) as a flocculent, off-white solid. ¹H NMR (300 MHz, DMSO-d₆) δ=7.67-7.47 (m, 14H), 6.52-6.39 (m, 2H), 4.17-4.01 (m, 4H), 3.87 (br s, 2H), 3.62 (br. t, J=5.4 Hz, 4H), 2.69-2.54 (m, 4H), 0.60 (s, 6H). ¹⁹F NMR (282 MHz, DMSO-d₆) δ=−113.86-−114.02 (m, 1F). LC/MS (M+H)⁺=576.

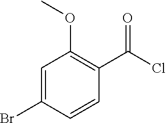

Synthesis of 4-bromo-2-methoxy-benzoyl chloride: To a suspension of 4-bromo-2-methoxy-benzoic acid (2.14 g, 9.26 mmol) and N,N-dimethylformamide (0.0677 g, 0.0714 mL, 0.926 mmol) in CHCl₃ (23 mL) at 0° C. under N₂ was added oxalyl chloride (1.76 g, 1.19 mL, 13.9 mmol) dropwise. The cooling bath warmed to 23° C. for 18 hours. The mixture was concentrated. The residue was dissolved in CHCl₃ and concentrated again, then dried under vacuum to yield the title compound, which was used without purification.

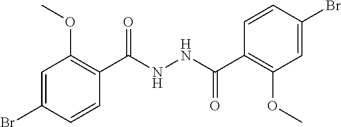 [SMILES: COc1cc(Br)ccc1C(=O)NNC(=O)c1ccc(Br)cc1OC]

Synthesis of 4-bromo-2-methoxy-benzoic acid N′-(4-bromo-2-methoxy-benzoyl)-hydrazide: To a solution of hydrazine hydrate (0.148 g, 0.224 mL, 4.63 mmol) and N,N-diisopropylethylamine (1.56 g, 2.10 mL, 12.0 mmol) in CHCl₃ (23 mL) at 0° C. under N₂ was added a solution of 4-bromo-2-methoxy-benzoyl chloride (9.26 mmol) in CHCl₃ (23 mL) dropwise via an addition funnel over 20 minutes. The cooling bath warmed to 23° C., and the reaction was stirred for 72 hours. The mixture was concentrated. The residue was diluted with water (30 mL), sonicated to break up the solid, then stirred at 23° C. for 18 hours. The water was decanted from the sticky solid, and ether (30 mL) was added. After brief sonication and stirring for 15 minutes, the solid was collected by filtration and washed with additional ether, then dried under vacuum to yield the title compound (1.9654 g, 93% over two steps) as a white solid.

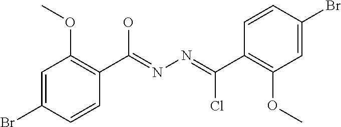 [SMILES: COc1cc(Br)ccc1/C(O)=N/N=C(\Cl)c1ccc(Br)cc1OC]

Synthesis of (Z)-4-bromo-N—[(Z)-(4-bromo-2-methoxyphenyl)(chloro)methylidene]-2-methoxybenzene-1-carbohydrazonoyl chloride: To a suspension of 4-bromo-2-methoxy-benzoic acid N′-(4-bromo-2-methoxy-benzoyl)-hydrazide (0.500 g, 1.09 mmol) in anhydrous toluene (6 mL) in a pressure tube at 23° C. under N₂ was gradually added PCl₅ (0.682 g, 3.27 mmol). The mixture was heated at 95° C. for 18 hours and then heated at 120° C. for four hours. The reaction was cooled to 23° C. and concentrated, suspended in toluene and concentrated again (2×), then dried under vacuum to yield the desired compound as a yellowish solid, which was used without purification.

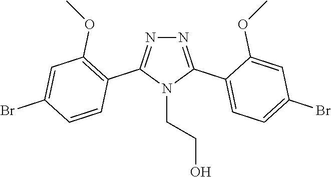 [SMILES: COc1cc(Br)ccc1-c1nnc(-c2ccc(Br)cc2OC)n1CCO]

Synthesis of 2-[3,5-bis-(4-bromo-2-methoxy-phenyl)-[1,2,4]triazol-4-yl]-ethanol: To a solution of (Z)-4-bromo-N—[(Z)-(4-bromo-2-methoxyphenyl)(chloro)methylidene]-2-methoxy benzene-1-carbohydrazonoyl chloride (1.09 mmol) in anhydrous toluene (6 mL) at 0° C. under N₂ was added 2-amino-ethanol (0.666 g, 0.656 mL, 10.9 mmol) dropwise. After 90 minutes, the reaction was permitted to warm to 23° C. and stirred for 72 hours. The reaction was cooled to 0° C., acetic acid (4 mL) was added, and the mixture was heated to 60° C. for two hours before cooling to 23° C. and concentrating. The residue was partitioned between 4:1 CHCl₃-isopropanol (150 mL) and saturated NaHCO₃ solution (50 mL) and separated. The aqueous layer was re-extracted with 4:1 CHCl₃-isopropanol (2×40 mL). The organic layers were combined and washed with saturated NaHCO₃ solution (25 mL), and brine (25 mL), then dried (Na₂SO₄), filtered, and concentrated. The residue was purified by reverse-phase chromatography (SiliaSep C_{18 b 80} g column; 5 to 100% MeCN-water (both with 0.1% trifluoroacetic acid) over 22 min.). The fractions containing the title compound were combined and partially concentrated (without heating) to remove most of the acetonitrile, then partitioned between 4:1 CHCl₃ isopropanol-isopropanol (80 mL) and saturated NaHCO₃ solution (30 mL) and separated. The aqueous layer was re-extracted with 4:1 CHCl₃-isopropanol (2×20 mL). The organic layers were combined and washed with brine (0.15 mL), then dried (Na₂SO₄), filtered, and concentrated to yield the title compound (0.0396 g, 5%) as a tan solid.

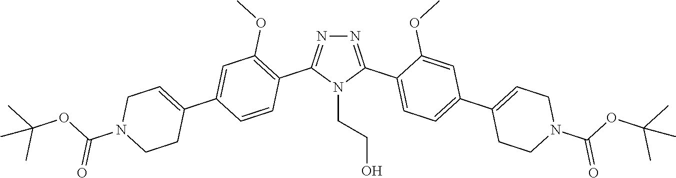 [SMILES: COc1cc(C2=CCN(C(=O)OC(C)(C)C)CC2)ccc1-c1nnc(-c2ccc(C3=CCN(C(=O)OC(C)(C)C)CC3)cc2OC)n1CCO]

Synthesis of tert-butyl 4-{4-[5-(4-{1-[(tert-butoxy)carbonyl]-1,2,3,6-tetrahydropyridin-4-yl}-2-methoxyphenyl)-4-(2-hydroxyethyl)-4H-1,2,4-triazol-3-yl]-3-methoxyphenyl}-1,2,3,6-tetrahydropyridine-1-carboxylate: A solution of 2-[3,5-bis-(4-bromo-2-methoxy-phenyl)-[1,2,4]triazol-4-yl]-ethanol (0.0396 g, 0.0820 mmol) in 1,4-dioxane (2.5 mL) was degassed by bubbling nitrogen through the mixture for 5 minutes. After adding tert-butyl 5,6-dihydro-4-(4,4,5,5-tetramethyl-1,3,2-dioxaborolan-2-yl)pyridine-1(2H)-carboxylate (0.0558 g, 0.180 mmol), 2 M Na₇CO₃ solution (0.205 mL, 0.410 mmol), and 1,1′-bis(diphenylphosphino)ferrocene]dichloropalladium(II), CH₂Cl₂ adduct (0.00669 g, 0.00820 mmol), the reaction was degassed for two more minutes, then heated at 80° C. for four hours. LC-MS showed primarily mono addition of the boronate ester-added additional (0.020 g) tert-butyl 5,6-dihydro-4-(4,4,5,5-tetramethyl-1,3,2-dioxaborolan-2-yl)pyridine-1(2H)-carboxylate, then heated the reaction at 80° C. for 18 hours. The mixture was cooled to 23° C. and partitioned between 4:1 CHCl₃-isopropanol (80 mL) and saturated NaHCO₃ solution (0.15 mL) and separated. The aqueous layer was re-extracted with 4:1 CHCl₃-isopropanol (2×20 mL). The organic layers were combined and washed with brine (15 mL), then dried (Na₂SO₄), filtered, and concentrated. Silica gel chromatography (0 to 100% (15% isopropanol-85% ethyl acetate); 100 to 0% hexanes), followed by concentration and drying under vacuum, yielded the title compound (0.0370 g, 66%) as a light brown foam.

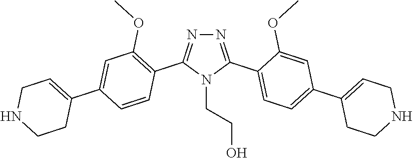 [SMILES: COc1cc(C2=CCNCC2)ccc1-c1nnc(-c2ccc(C3=CCNCC3)cc2OC)n1CCO]

Synthesis of 2-{3,5-bis-[2-methoxy-4-(1,2,3,6-tetrahydro-pyridin-4-yl)-phenyl]-[1,2,4]triazol-4-yl}-ethanol hydrochloride: To a solution of tert-butyl 4-{4-[5-(4-{1-[(tert-butoxy) carbonyl]-1,2,3,6-tetrahydropyridin-4-yl}-2-methoxyphenyl)-4-(2-hydroxyethyl)-4H-1,2,4-triazol-3-yl]-3-methoxyphenyl}-1,2,3,6-tetrahydropyridine-1-carboxylate (0.0370 g, 0.0538 mmol) and anisole (0.0582 g, 0.0586 mL, 0.538 mmol) in dichlorourethane (3 mL) at 23° C. under N₂ was added 4 N HCl-1,4-dioxane solution (1.00 mL, 4.00 mmol) dropwise. A precipitate began to form—the vial was occasionally sonicated to keep it suspended. After three hours, the mixture was concentrated, then suspended in CH₂Cl₂ and concentrated again. The residue was dissolved in 4:1 MeCN-water, frozen at −78° C., then lyophilized to yield the title compound (0.0329 g, quantitative) as a tan solid.

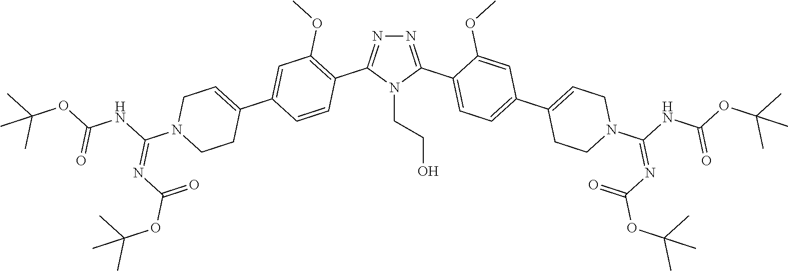 [SMILES: COc1cc(C2=CCN(/C(=N\C(=O)OC(C)(C)C)NC(=O)OC(C)(C)C)CC2)ccc1-c1nnc(-c2ccc(C3=CCN(/C(=N\C(=O)OC(C)(C)C)NC(=O)OC(C)(C)C)CC3)cc2OC)n1CCO]

Synthesis of N—[(Z)-(4-{4-[5-(4-{1-[(Z)-{[(tert-butoxy)carbonyl]amino}({[(tert-butoxy)carbonyl]imino})methyl]-1,2,3,6-tetrahydropyridin-4-yl}-2-methoxyphen yl)-4-(2-hydroxyethyl)-4H-1,2,4-triazol-3-yl]-3-methoxyphenyl}-1,2,3,6-tetrahydropyridin-1-yl)({[(tert-butoxy)carbonyl]imino})methyl]carbamate: To a solution of 2-{3,5-bis-[2-methoxy-4-(1,2,3,6-tetrahydro-pyridin-4-yl)-phenyl]-[1,2,4]triazol-4-yl}-ethanol hydrochloride (0.0329 g, 0.0551 mmol) and N,N-diisopropylethylamine (0.0499 g, 0.0672 mL, 0.386 mmol) in N,N dimethylformamide (2 mL) at 23° C. under N₂ was added tert-butyl N—[(Z)-{[(tert-butoxy)carbonyl]imino}(1H-pyrazol-1-yl)methyl]carbamate (0.0513 g, 0.165 mmol). The reaction was stirred at 23° C. for 18 hours. LC-MS analysis showed a significant amount of mono-guanylation. The mixture was heated at 40° C. for 0.18 hours. After cooling to 23° C., the mixture was partitioned between ethyl acetate (130 mL) and saturated NH₄Cl solution (15 mL) and separated. The organic layer was washed with water (15 mL), saturated NaHCO₃ solution (15 mL), and brine (15 mL), then dried (Na₂SO₄), filtered, and concentrated. Silica gel chromatography (0 to 100% (25% isopropanol-75% ethyl acetate); 100 to 0% hexanes), followed by concentration and drying under vacuum, yielded the title compound (0.0308 g, 58%) as a tan solid.

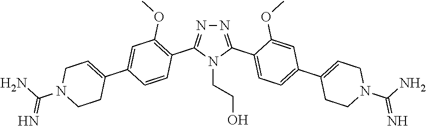 [SMILES: COc1cc(C2=CCN(C(=N)N)CC2)ccc1-c1nnc(-c2ccc(C3=CCN(C(=N)N)CC3)cc2OC)n1CCO]

### Example 75

Synthesis of 4-(4-{5-[4-(1-carbamimidoyl-1,2,3,6-tetrahydropyridin-4-yl)-2-methoxyphenyl]-4-(2-hydroxyethyl)-4H-1,2,4-triazol-3-yl}-3-methoxyphenyl)-1,2,3,6-tetrahydropyridine-1-carboximidamide trifluoroacetate: To a solution of tert-butyl N—[(Z)-(4-{4-[5-(4-{1-[(Z)-{[(tert-butoxy)carbonyl]amino}({[(tert-butoxy)carbonyl]imino})methyl]-1,2,3,6-tetrahydropyridin-4-yl}-2-methoxyphenyl)-4-(2-hydroxyethyl)-4H-1,2,4-triazol-3-yl]-3-methoxyphenyl}-1,2,3,6-tetrahydropyridin-1-yl)({[(tert-butoxy)carbonyl]imino})methyl]carbamate (0.0308 g, 0.0317 mmol) and anisole (0.0343 g, 0.0345 mL, 0.317 mmol) in dichloromethane (2 mL) at 23° C. under N₂ was added 4 N HCl-1,4-dioxane solution (1.00 mL, 4.00 mmol) dropwise. A precipitate formed the vial was sonicated to keep it suspended. After three hours, the mixture was concentrated, then suspended in CH₂Cl₂ and concentrated again. The residue was purified by reverse-phase chromatography (SiliaSep C₁₈ 12 g column; 5 to 100% MeCN-water (both with 0.1% trifluoroacetic acid) over 17 min.). The fractions containing the title compound were combined and partially concentrated (without heating) to remove most of the acetonitrile, then lyophilized to yield the title compound (0.0042 g, 17%) as a flocculent, white solid. ¹H NMR (300 MHz, DMSO-d₆) δ ppm 2.59-2.70 (m, 4H) 3.06 (br t, J=6.15 Hz, 2H) 3.63 (br t, J=5.27 Hz, 4H) 3.74 (br t, J=5.86 Hz, 2H) 3.86 (s, 6H) 4.10 (m, J=2.30 Hz, 4H) 6.32-6.44 (m, 2H) 7.14-7.27 (m, 4H) 7.35-7.52 (m, 10H). LC/MS (M+H)⁺572.

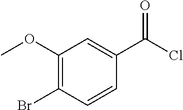

Synthesis of 4-bromo-3-methoxy-benzoyl chloride: To a suspension of 4-bromo-3-methoxy-benzoic acid (2.14 g, 9.26 mmol) and N,N-dimethylformamide (0.0677 g, 0.0714 mL, 0.926 mmol) in CHCl₃ (23 mL) at 0° C. under N₂ was added oxalyl chloride (1.76 g, 1.19 mL, 13.9 mmol) dropwise. The cooling bath warmed to 23° C. for 18 hours. The mixture was concentrated. The residue was dissolved in CHCl₃ and concentrated again (2×), then dried under vacuum to yield the title compound as a yellowish solid, which was used without purification.

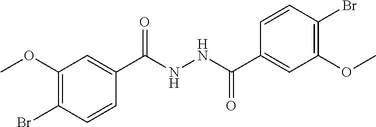

Synthesis of 4-bromo-3-methoxy-benzoic acid N′-(4-bromo-3-methoxy-benzoyl)-hydrazide: To a solution of hydrazine hydrate (0.148 g, 0.224 mL, 4.63 mmol) and N,N-diisopropylethylamine (1.56 g, 2.10 mL, 12.0 mmol) in CHCl₃ (10 mL) at 0° C. under N₂ was added a solution of 4-bromo-3-methoxy-benzoyl chloride (9.26 mmol) in CHCl₃ (23 mL) dropwise via an addition funnel over 20 minutes. The cooling bath warmed to 23° C., and the reaction was stirred for 72 hours. The mixture was concentrated, then suspended in CHCl₃ and concentrated again. The residue was suspended in ether (30 mL) and stirred vigorously for 30 minutes, sonicating occasionally. The solid was collected by filtration and washed with additional ether, then dried under vacuum at 45° C. for 18 hours to yield a grayish solid. The solid was suspended in water (30 mL) and stirred vigorously for 30 minutes, sonicating occasionally. The solid was again collected by filtration, washed with water, then ether, then dried under vacuum to yield the title product (1.9137 g, 90% over two steps) as a slightly gray solid.

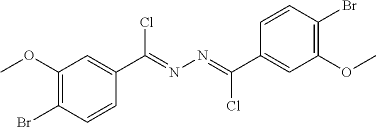

Synthesis of (Z)-4-bromo-N—[(Z)-(4-bromo-3-methoxyphenyl)(chloro)methyl idene]-3-methoxybenzene-1-carbohydrazonoyl chloride: To a suspension of 4-bromo-3-methoxy-benzoic acid N′-(4-bromo-3-methoxy-benzoyl)-hydrazide (0.500 g, 1.09 mmol) in anhydrous toluene (5 mL) at 23° C. under N₂ was gradually added PCl₅ (0.682 g, 3.27 mmol). The mixture was heated at 100° C. for 18 hours and then 120° C. for four hours. The reaction was cooled to 23° C. and concentrated, suspended in CH₂Cl₂ and concentrated again, then dried under vacuum to yield the title compound as a yellow solid, which was used without purification.

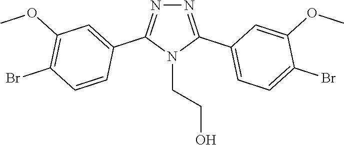

Synthesis of 2-[3,5-bis-(4-bromo-3-methoxy-phenyl)-[1,2,4]triazol-4-yl]-ethanol (1.09 mmol) in anhydrous toluene (6 mL) at 0° C. under N₂ was added 2-amino-ethanol (0.666 g, 0.656 mL, 10.9 mmol) dropwise. After 90 minutes, the reaction was permitted to warm to 23° C. and stirred for 72 hours. Acetic acid (2 mL) was added, and the mixture was heated to 60° C. for one hour before cooling to 23° C. and concentrating. The residue was partitioned between ethyl acetate (140 mL) and saturated NaHCO₃ solution (40 mL) and separated. The organic layer was washed with saturated NaHCO₃ solution (15 mL), and brine (15 mL), then dried (Na₂SO₄), filtered, and concentrated. Silica gel chromatography (0 to 100% (15% isopropanol-85% ethyl acetate); 100 to 0% hexanes), followed by concentration and drying under vacuum, yielded the title compound (0.3738 g, 71%) as a white foam.

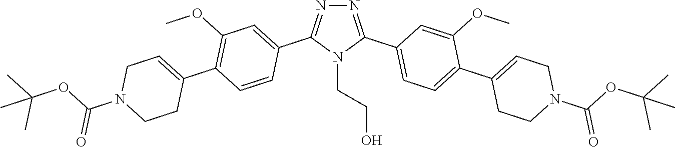 [SMILES: COc1cc(-c2nnc(-c3ccc(C4=CCN(C(=O)OC(C)(C)C)CC4)c(OC)c3)n2CCO)ccc1C1=CCN(C(=O)OC(C)(C)C)CC1]

Synthesis of tert-butyl 4-{4-[5-(4-{1-[(tert-butoxy)carbonyl]-1,2,3,6-tetrahydropyridin-4-yl}-3-methoxyphenyl)-4-(2-hydroxyethyl)-4H-1,2,4-triazol-3-yl]-2-methoxyphenyl}-1,2,3,6-tetrahydropyridine-1-carboxylate: A solution of tert-butyl 5,6-dihydro-4-(4,4,5,5-tetramethyl-1,3,2-dioxaborolan-2-yl)pyridine-1(2H)-carboxylate (0.141 g, 0.455 mmol) in 1,4-dioxane (2.5 mL) in a scintillation vial was degassed by bubbling nitrogen through the mixture for several minutes. After adding 2 M Na₂CO₃ solution (0.517 mL, 1.03 mmol), 2-[3,5-bis-(4-bromo-3-methoxy-phenyl)-[1,2,4]triazol-4-yl]-ethanol (0.100 g, 0.207 mmol), and 1,1′-bis(diphenylphosphino)ferrocene] dichloropalladium(II), CH₂Cl₂ adduct (0.0169 g, 0.0207 mmol), the reaction was degassed for two more minutes, heated at 80° C. for three hours, then stirred at 23° C. for 18 hours. The mixture was partitioned between CH₂Cl₂ (80 mL) and saturated NaHCO₃ solution (20 mL) and separated. The aqueous layer was re-extracted with CH₂Cl₂ (2×20 mL). The organic layers were combined and washed with brine (15 mL), then dried (Na₂SO₄), filtered, and concentrated. Silica gel chromatography (0 to 100% (15% isopropanol-85% ethyl acetate); 100 to 0% hexanes), followed by concentration and drying under vacuum, yielded a dark oil. This residue was further purified by reverse-phase chromatography (SiliaSep C₁₈ 40 g column; 5 to 100% MeCN-water (both with 0.1% trifluoroacetic acid) over 22 min.). The fractions containing the title compound were combined and partially concentrated (without heating) to remove most of the acetonitrile, then partitioned between 4:1 CHCl₃-isopropanol (80 mL) and saturated NaHCO₃ solution (35 mL) and separated. The aqueous layer was re-extracted with 4:1 CHCl₃-isopropanol (2×20 mL). The organic layers were combined and washed with brine (15 mL), then dried (Na₂SO₄), filtered, concentrated, and dried under vacuum to yield the title compound (0.0666 g, 47%) as an off-white foam.

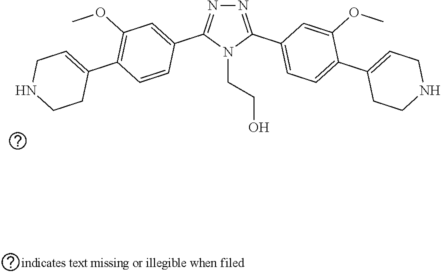 [SMILES: COc1cc(-c2nnc(-c3ccc(C4=CCNCC4)c(OC)c3)n2CCO)ccc1C1=CCNCC1]

Synthesis of 2-{3,5-bis-[3-methoxy-4-(1,2,3,6-tetrahydro-pyridin-4-yl)-phenyl]-[1,2,4]triazol-4-yl}-ethanol hydrochloride: To a solution of tert-butyl 4-{4-[5-(4-{1-[(tert-butoxy) carbonyl]-1,2,3,6-tetrahydropyridin-4-yl}-3-methoxyphenyl)-4-(2-hydroxyethyl)-4H-1,2,4-triazol-3-yl]-2-methoxyphenyl}-1,2,3,6-tetrahydropyridine-1-carboxylate (0.0650 g, 0.0945 mmol) and anisole (0.102 g, 0.130 mL, 0.0945 mmol) in dichloromethane (2 mL) at 23° C. under N₂ was added 4 N HCl . . . 1,4-dioxane solution (1.00 mL, 4.00 mmol) dropwise. A precipitate began to form—the vial was occasionally sonicated to keep it suspended. After three hours, the mixture was concentrated, then suspended in CH₂Cl₂ and concentrated again. The residue was dissolved in 4:1 MeCN-water, frozen at −78° C., then lyophilized to yield the title compound (0.0526 g, 93%) as an off-white solid.

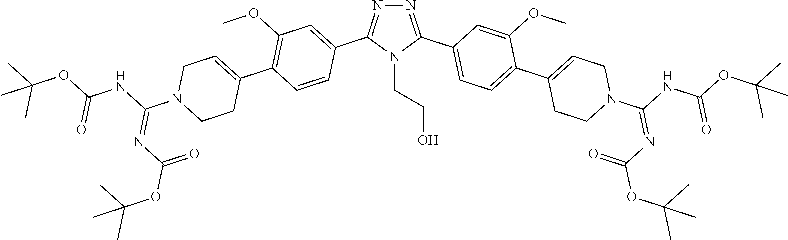 [SMILES: COc1cc(-c2nnc(-c3ccc(C4=CCN(/C(=N\C(=O)OC(C)(C)C)NC(=O)OC(C)(C)C)CC4)c(OC)c3)n2CCO)ccc1C1=CCN(/C(=N\C(=O)OC(C)(C)C)NC(=O)OC(C)(C)C)CC1]

Synthesis of tert-butyl N—[(Z)-(4-{4-[5-(4-{1-[(Z)-{[(tert-butoxy)carbonyl]amino}({[(tert-butoxy)carbonyl]imino})methyl]-1,2,3,6-tetrahydropyridin-4-yl}-3-methoxyphen yl)-4-(2-hydroxyethyl)-4H-1,2,4-triazol-3-yl]-2-methoxyphenyl}-1,2,3,6-tetrahydropyridin-1-yl)({[(tert-butoxy)carbonyl]imino})methyl]carbamate: To a suspension of 2-{3,5-bis-[3-methoxy-4-(1,2,3,6-tetrahydro-pyridin-4-yl)-phenyl]-[1,2,4]triazol-4-yl}-ethanol hydrochloride (0.0526 g, 0.0881 mmol) and N,N-diisopropylethylamine (0.0797 g, 0.107 mL, 0.0617 mmol) in N,N dimethylformamide (2 mL) at 23° C. under N₂ was added tert-butyl N—[(Z)-{[(tert-butoxy)carbonyl]imino}(1H-pyrazol-1-yl)methyl]carbamate (0.0820 g, 0.264 mmol). The reaction was stirred at 23° C. for 18 hours. The mixture was heated at 40° C. for four hours, then at 45° C. for 18 hours. An additional portion (0.0820 g, 0.264 mmol) of tert-butyl N—[(Z)-{[(tert-butoxy)carbonyl]imino}(1H-pyrazol-1-yl)methyl]carbamate was added, and heating was continued for 72 hours. After cooling to 23° C., the mixture was partitioned between ethyl acetate (130 mL) and saturated NH₄Cl solution (15 mL) and separated. The organic layer was washed with water (15 mL), saturated NaHCO₃ solution (15 mL), and brine (15 mL), then dried (Na₂SO₄), filtered, and concentrated. Silica gel chromatography (0 to 100% (25% isopropanol-75% ethyl acetate); 100 to 0% hexanes), followed by concentration and drying under vacuum, yielded the title compound (0.0294 g, 33%) as an off-white solid.

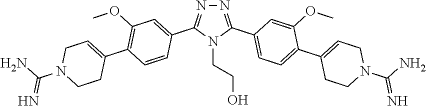 [SMILES: COc1cc(-c2nnc(-c3ccc(C4=CCN(C(=N)N)CC4)c(OC)c3)n2CCO)ccc1C1=CCN(C(=N)N)CC1]

### Example 76

Synthesis of 4-(4-{5-[4-(1-carbamimidoyl-1,2,3,6-tetrahydropyridin-4-yl)-3-methoxyphenyl]-4-(2-hydroxyethyl)-4H-1,2,4-triazol-3-yl}-2-methoxyphenyl)-1,2,3,6-tetrahydropyridine-1-carboximidamide trifluoroacetate: To a solution of tert-butyl N—[(Z)-(4-{4-[5-(4-{1-[(Z)-{[(tert-butoxy)carbonyl]amino}({[(tert-butoxy)carbonyl]imino})methyl]-1,2,3,6-tetrahydropyridin-4-yl}-3-methoxyphenyl)-4-(2-hydroxyethyl)-4H-1,2,4-triazol-3-yl]-2-methoxyphenyl}-1,2,3,6-tetrahydropyridin-1-yl)({[(tert-butoxy)carbonyl]imino})methyl]carbamate (0.0286 g, 0.0294 mmol) and anisole (0.0318 g, 0.0320 mL, 0.294 mmol) in dichloromethane (2 mL) at 23° C. under N₂ was added 4 N HCl-dioxane solution (1.00 mL, 4.00 mmol) dropwise. A precipitate began to form—the vial was occasionally sonicated to keep it suspended. After four hours, the mixture was concentrated, suspended in CH₂Cl₂ and concentrated again, then lyophilized. The residue was purified by reverse-phase chromatography (SiliaSep C_{18 b 40} g column; 5 to 45% MeCN-water (both with 0.1% trifluoroacetic acid) over 23 min.). The fractions containing the title compound were combined and lyophilized to yield the title compound (0.0100 g, 34%) as a flocculent, white solid. ¹H NMR (300 MHz, DMSO-d₆) δ ppm 7.42 (s, 8H) 7.25-7.37 (m, 6H) 5.87-5.99 (m, 2H) 4.23 (m, J=5.00, 5.00, 1.80 Hz, 2H) 4.04 (m, J=1.80 Hz, 4H) 3.82 (s, 6H) 3.56 (br t, J=5.27 Hz, 4H) 3.26-3.30 (m, 2H) 2.5.1-2.60 (m, 4H). ¹⁹F NMR (282 MHz, DMSO-d₆) δ ppm −73.71 (s, 1F). LC/MS (M H)⁺=572.

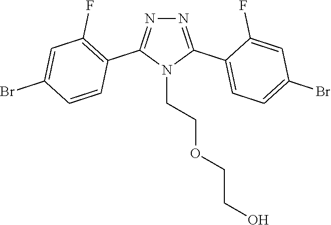 [SMILES: OCCOCCn1c(-c2ccc(Br)cc2F)nnc1-c1ccc(Br)cc1F]

Synthesis of 2-{2-[3,5-bis-(4-bromo-2-fluoro-phenyl)-[1,2,4]triazol-4-yl]-ethoxy}-ethanol: To a solution of (Z)-4-bromo-N—[(Z)-(4-bromo-2-fluorophenyl)(chloro)methylidene]-2-fluorobenzene-1-carbohydrazonoyl chloride (1.15 mmol) in anhydrous toluene (5 mL) at 0° C. under N₂ was added 2-(2-amino-ethoxy)-ethanol (1.21 g, 1.15 mL, 11.5 mmol) dropwise. After 90 minutes, the reaction was permitted to warm to 23° C. and stirred for 18 hours. Acetic acid (2 mL) was added, and the mixture was heated to 60° C. for 1.5 hours before cooling to 23° C. and concentrating. The residue was partitioned between ethyl acetate (250 mL) and saturated NaHCO₃ solution (35 mL) and separated. The organic layer was washed with water (2×25 mL), and brine (25 mL), then dried (Na₂SO₄), filtered, and concentrated. Silica gel chromatography (0 to 100% (25% isopropanol-75% ethyl acetate); 100 to 0% hexanes), followed by concentration and drying under vacuum, yielded the title compound (0.4290 g, 74%) as a white solid.

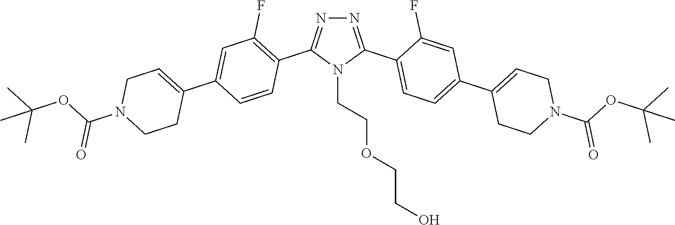 [SMILES: CC(C)(C)OC(=O)N1CC=C(c2ccc(-c3nnc(-c4ccc(C5=CCN(C(=O)OC(C)(C)C)CC5)cc4F)n3CCOCCO)c(F)c2)CC1]

Synthesis of tert-butyl 4-{4-[5-(4-{1-[(tert-butoxy)carbonyl]-1,2,3,6-tetrahydropyridin-4-yl}-2-fluorophenyl)-4-[2-(2-hydroxyethoxy)ethyl]-4H-1,2,4-triazol-3-yl]-3-fluorophenyl}-1,2,3,6-tetrahydropyridine-1-carboxylate: A solution of tert-butyl 5,6-dihydro-4-(4,4,5,5-tetramethyl-1,3,2-dioxaborolan-2-yl)pyridine-1(2H)-carboxylate (0.135 g, 0.437 mmol) in 1,4-dioxane (2.5 mL) was degassed by bubbling nitrogen through the mixture for 5 minutes. After adding 2 M Na₂CO₃ solution (0.497 mL, 0.994 mmol), 2-{2-[3,5-bis-(4-bromo-2-fluoro-phenyl)-[1,2,4]triazol-4-yl]-ethoxy}-ethanol (0.100 g, 0.199 mmol), and 1,1′-bis(diphenylphosphino)ferrocene]dichloropalladium(II), CH₂Cl₂ adduct (0.0162 g, 0.0199 mmol), the reaction was degassed for two more minutes, heated at 80° C. for three hours, then stirred at 23° C. for 18 hours. The mixture was partitioned between CH₂Cl₂ (80 mL) and saturated NaHCO₃ solution (20 mL) and separated. The aqueous layer was re-extracted with CH₂Cl₂ (2×20 mL). The organic layers were combined and washed with brine (15 mL), then dried (Na₂SO₄), filtered, and concentrated. Silica gel chromatography (0 to 100% (25% isopropanol-75% ethyl acetate); 100 to 0% hexanes), followed by concentration and drying under vacuum, yielded the title compound (0.1121 g, 80%) as a tan foam.

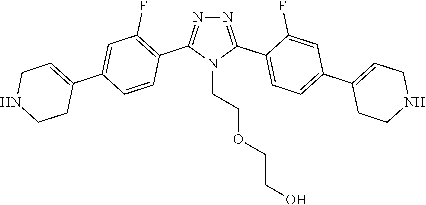 [SMILES: OCCOCCn1c(-c2ccc(C3=CCNCC3)cc2F)nnc1-c1ccc(C2=CCNCC2)cc1F]

Synthesis of 2-(2-{3,5-bis-[2-fluoro-4-(1,2,3,6-tetrahydro-pyridin-4-yl)-phenyl]-[1,2,4]triazol-4-yl}-ethoxy)-ethanol hydrochloride: To a solution of tert-butyl 4-{4-[5-(4-{1-[(tert-butoxy)carbonyl]-1,2,3,6-tetrahydropyridin-4-yl}-2-fluorophenyl)-4-[2-(2-hydroxyethoxy)ethyl]-4H-1,2,4-triazol-3-yl]-3-fluorophenyl}-1,2,3,6-tetrahydropyridine-1-carboxylate (0.110 g, 0.155 mmol) and anisole (0.168 g, 0.169 mL, 0.1.55 mmol) in dichloromethane (2.5 mL) in a scintillation vial at 23° C. under N₂ was added 4 N HCl-1,4-dioxane solution (1.00 mL, 4.00 mmol) dropwise. A precipitate began to form—the vial was occasionally sonicated to keep it suspended. After three hours, the mixture was concentrated, then suspended in CH₂Cl₂ and concentrated again. The residue was dissolved in 4:1 MeCN-water, frozen at −78° C., then lyophilized to yield the title compound 0.0885 g, 93% as a flocculent, light brown solid.

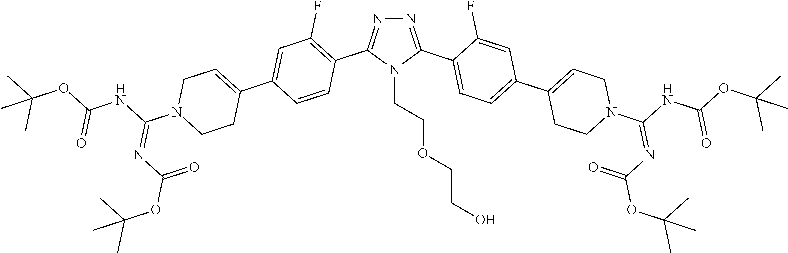 [SMILES: CC(C)(C)OC(=O)/N=C(/NC(=O)OC(C)(C)C)N1CC=C(c2ccc(-c3nnc(-c4ccc(C5=CCN(/C(=N\C(=O)OC(C)(C)C)NC(=O)OC(C)(C)C)CC5)cc4F)n3CCOCCO)c(F)c2)CC1]

Synthesis of tert-butyl N—[(Z)-(4-{4-[5-(4-{1-[(Z)-{[(tert-butoxy)carbonyl]amino}({[(tert-butoxy)carbonyl]imino})methyl]-1,2,3,6-tetrahydropyridin-4-yl}-2-fluorophenyl)-4-[2-(2-hydroxyethoxy)ethyl]-4H-1,2,4-triazol-3-yl]-3-fluorophenyl}-1,2,3,6-tetrahydropyridin-1-yl)({[(tert-butoxy)carbonyl]imino})methyl]carbamate: To a solution of 2-(2-(3,5-bis-[2-fluoro-4-(1,2,3,6-tetrahydro-pyridin-4-yl)-phenyl]-[1,2,4]triazol-4-yl)-ethoxy)-ethanol hydrochloride (0.0847 g, 0.137 mmol) and N,N-diisopropylethylamine (0.124 g, 0.167 mL, 0.961 mmol) in N,N dimethylformamide (2 mL) in a scintillation vial at 23° C. under N₂ was added tert-butyl N—[(Z)-{[(tert-butoxy)carbonyl]imino}(1H-pyrazol-1-yl)methyl]carbamate (0.128 g, 0.412 mmol). The reaction was stirred at 23° C. for 72 hours. The mixture was partitioned between ethyl acetate (130 mL) and saturated NH₄Cl solution (15 mL) and separated. The organic layer was washed with water (15 saturated NaHCO₃ solution (15 mL), and brine (15 mL), then dried (Na₂SO₄), filtered, and concentrated. The aqueous layers was re-extracted with 4:1 CHCl₃-isopropanol (3×25 mL). The organic layers were combined and washed with brine (15 mL), then dried (Na₂SO₄), filtered, combined with the ethyl acetate-extracted material, and concentrated. Silica gel chromatography (0 to 100% (25% isopropanol-75% ethyl acetate); 100 to 0% hexanes), followed by concentration and drying under vacuum, yielded the title compound (0.0686 g, 51%) as an off-white solid.

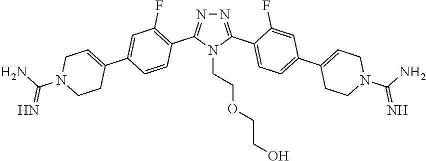 [SMILES: N=C(N)N1CC=C(c2ccc(-c3nnc(-c4ccc(C5=CCN(C(=N)N)CC5)cc4F)n3CCOCCO)c(F)c2)CC1]

### Example 77

Synthesis of 4-(4-{5-[4-(1-carbamimidoyl-1,2,3,6-tetrahydropyridin-4-yl)-2-fluorophenyl]-4-[2-(2-hydroxyethoxy)ethyl]-4H-1,2,4-triazol-3-yl}-3-fluorophen yl)-1,2,3,6-tetrahydropyridine-1-carboximidamide trifluoroacetate: To a solution of tert-butyl N—[(Z)-(4-{4-[5-(4-{1-[(Z)-{[(tert-butoxy)carbonyl]amino}({[(tert-butoxy)carbonyl]imino})methyl]-1,2,3,6-tetrahydropyridin-4-yl}-2-fluorophenyl)-4-[2-(2-hydroxyethoxy)ethyl]-4H-1,2,4-triazol-3-yl]-3-fluorophenyl}-1,2,3,6-tetrahydropyridin-1-yl)({[(tert-butoxy)carbonyl]imino})methyl]carbamate (0.0660 g, 0.0665 mmol) and anisole (0.0719 g, 0.0724 mL, 0.665 mmol) in dichloromethane (3 mL) in a scintillation vial at 23° C. under N₂ was added 4 N HCl-1,4-dioxane solution (1.00 mL, 4.00 mmol) dropwise. A precipitate began to form—the vial was occasionally sonicated to keep it suspended. After two hours, the mixture was concentrated, suspended in CH₂Cl₂ and concentrated again, then dissolved in 4:1 MeCN-water, and lyophilized. The residue was purified by reverse-phase chromatography (SiliaSep C_{18 b 40} g column; 5 to 70% MeCN-water (both with 0.1% trifluoroacetic acid) over 23 min.). The fractions containing the title compound were combined and partially concentrated (without heating) to remove most of the acetonitrile, then lyophilized to yield the title compound (0.0332 g, 61%) as a flocculent, white solid. ¹H NMR (300 MHz, DMSO-d₆) δ ppm 7.62-7.72 (m, 2H) 7.41-7.62 (m, 12H) 6.46 (br s, 2H) 4.10 (m, J=1.20 Hz, 4H) 4.01 (br t, J=4.69 Hz, 2H) 3.63 (br t, J=5.27 Hz, 4H) 3.14-3.22 (m, 4H) 2.95-3.02 (m, 2H) 2.57-2.69 (m, 4H). ¹⁹F NMR (282 MHz, DMSO-d₆) δ ppm −73.75 (s, 1F) −113.89-−113.75 (m, 1F). LC/MS (M+H)⁺=592.

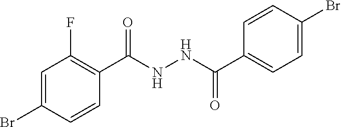 [SMILES: O=C(NNC(=O)c1ccc(Br)cc1F)c1ccc(Br)cc1]

Synthesis of 4-bromo-benzoic acid N′-(4-bromo-2-fluoro-benzoyl)-hydrazide: To a suspension of 4-bromo-benzoic acid hydrazide (1.81 g, 8.42 mmol) and N,N-diisopropylethylamine (1.42 g, 1.91 mL, 10.9 mmol) in CHCl₃ (21 mL) at 0° C. under N₂ was added a solution of 4-bromo-2-fluoro-benzoyl chloride (2.00 g, 8.42 mmol) in CHCl₃ (21 mL) dropwise via an addition funnel over 45 minutes. The cooling bath warmed to 23° C., and the reaction was stirred for 72 hours. The mixture (a white, viscous suspension) was concentrated. The residue was diluted with water (30 mL), sonicated to disperse the solid, then stirred for two hours with occasional sonication. The water was decanted from the gummy solid, and MeOH (10 mL) was added. After brief sonication and stirring, additional MeOH (20 mL) was added. The suspension was stirred for 20 minutes before the solid was collected by filtration and washed with additional MeOH, then ether, then dried under vacuum to yield the title compound (3.3879 g, 97%) as a white solid.

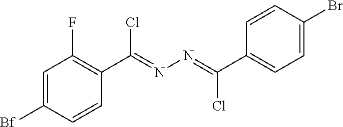

Synthesis of (Z)-4-bromo-N—[(Z)-(4-bromo-2-fluorophenyl)(chloro)methylidene]benzene-1-carbohydrazonoyl chloride: To a suspension of 4-bromo-benzoic acid N′-(4-bromo-2-fluoro-benzoyl)-hydrazide (0.300 g, 0.721 mmol) in anhydrous toluene (3.6 mL) in a pressure tube at 23° C. under N₂ was gradually added PCl₅ (0.450 g, 2.16 mmol). The mixture was heated at 95° C. for 18 hours. The reaction was cooled to 23° C. and concentrated, suspended in toluene and concentrated again, then dried under vacuum to yield the title compound as a yellowish solid, which was used without purification.

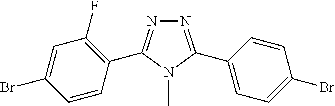 [SMILES: Cn1c(-c2ccc(Br)cc2)nnc1-c1ccc(Br)cc1F]

Synthesis of 3-(4-bromo-2-fluoro-phenyl)-5-(4-bromo-phenyl)-4-methyl-4H-[1,2,4]triazole: To a solution of (Z)-4-bromo-N—[(Z)-(4-bromo-2-fluorophenyl)(chloro)methylidene]benzene-1-carbohydrazonoyl chloride (0.721 mmol) in anhydrous toluene (5 mL) in a large scintillation vial at 0° C. under N₂ was added methylamine solution (33% in EtOH, 0.898 mL, 7.21 mmol) dropwise. After 60 minutes, the reaction was permitted to warm gradually to 23° C. and stirred for 18 hours. Acetic acid (1 mL) was added, and the mixture was heated to 60° C. for one hour before cooling to 23° C. and concentrating. The residue was partitioned between ethyl acetate (130 mL) and saturated NaHCO₃ solution (30 mL) and separated. The organic layer was washed with saturated NaHCO₃ solution (15 mL), water (15 mL), and brine (15 mL), then dried (Na₂SO₄), filtered, and concentrated. Silica gel chromatography (0 to 100% ethyl acetate; 100 to 0% hexanes), followed by concentration and drying under vacuum, yielded the title compound (0.2207 g, 75%) as a white solid.

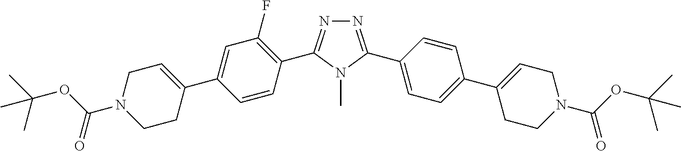 [SMILES: Cn1c(-c2ccc(C3=CCN(C(=O)OC(C)(C)C)CC3)cc2)nnc1-c1ccc(C2=CCN(C(=O)OC(C)(C)C)CC2)cc1F]

Synthesis of tert-butyl 4-{4-[5-(4-{1-[(tert-butoxy)carbonyl]-1,2,3,6-tetrahydropyridin-4-yl}-2-fluorophenyl)-4-methyl-4H-1,2,4-triazol-3-yl]phenyl}-1,2,3,6-tetrahydropyridine-1-carboxylate: A suspension of 3-(4-bromo-2-fluoro-phenyl)-5-(4-bromo-phenyl)-4-methyl-4H-[1,2,4]triazole (0.100 g, 0.243 mmol) in 1,4-dioxane (2.5 mL) was degassed by bubbling nitrogen through the mixture for 5 minutes. After adding 2 M Na₂CO₃ solution (0.608 mL, 1.22 mmol), tert-butyl 5,6-dihydro-4-(4,4,5,5-tetramethyl-1,3,2-dioxaborolan-2-yl)pyridine-1(2H)-carboxylate (0.158 g, 0.511 mmol), and 1,1′-bis(diphenylphosphino)ferrocene]dichloropalladium(II), CH₂Cl₂ adduct (0.0199 g, 0.0243 mmol), the reaction was degassed for two more minutes, then heated at 80° C. for 18 hours. The mixture was cooled to 23° C. and partitioned between CH₂Cl₂ (80 mL) and saturated NaHCO₃ solution (15 mL) and separated. The aqueous layer was re-extracted with CH₂Cl₂ (2×20 mL). The organic layers were combined and washed with brine (15 mL), then dried (Na₂SO₄), filtered, and concentrated. Silica gel chromatography (0 to 100% (15% isopropanol 85% ethyl acetate); 100 to 0% hexanes), followed by concentration and drying under vacuum, yielded the title compound (0.143 g, 59%) as a tan solid.

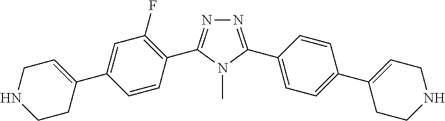 [SMILES: Cn1c(-c2ccc(C3=CCNCC3)cc2)nnc1-c1ccc(C2=CCNCC2)cc1F]

Synthesis of 4-(4-{5-[2-fluoro-4-(1,2,3,6-tetrahydropyridin-4-yl)phenyl]-4-methyl-4H-1,2,4-triazol-3-yl}phenyl)-1,2,3,6-tetrahydropyridine trifluoroacetate: To a solution of tert-butyl 4-{4-[5-(4-{1-[(tert-butoxy)carbonyl]-1,2,3,6=tetrahydropyridin-4-yl}-2-fluorophenyl)-4-methyl-4H-1,2,4-triazol-3-yl]phenyl}-1,2,3,6-tetrahydropyridine-1-carboxylate (0.0860 g, 0.140 mmol) and anisole (0.151 g, 0.152 mL, 1.40 mmol) in dichloromethane (2 mL) in a scintillation vial at 23° C. under N₂ was added trifluoroacetic acid (0.5 mL, 6.53 mmol) dropwise. After two hours, the mixture was concentrated, then suspended in CH₂Cl₂ and concentrated again. The residue was dissolved in 4:1 MeCN water, frozen at −78° C., then lyophilized to yield the title compound (0.1052 g, quantitative) as a flocculent, off-white solid.

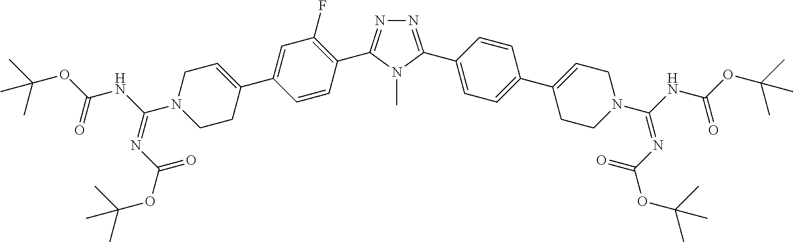 [SMILES: Cn1c(-c2ccc(C3=CCN(/C(=N\C(=O)OC(C)(C)C)NC(=O)OC(C)(C)C)CC3)cc2)nnc1-c1ccc(C2=CCN(/C(=N\C(=O)OC(C)(C)C)NC(=O)OC(C)(C)C)CC2)cc1F]

Synthesis of tert-butyl N—[(Z)-(4-{4-[5-(4-(1-[(Z)-{[(tert-butoxy)carbonyl]amino}({[(tert-butoxy)carbonyl]imino})methyl]-1,2,3,6-tetrahydropyridin-4-yl}-2-fluorophenyl)-4-methyl-4H-1,2,4-triazol-3-yl]phenyl}-1,2,3,6-tetrahydropyridin-1-yl)({[(tert-butoxy)carbonyl]imino})methyl]carbamate: To a solution of 4-(4-{5-[2-fluoro-4-(1,2,3,6-tetrahydropyridin-4-yl)phenyl]-4-methyl-4H-1,2,4-triazol-3-yl}phenyl)-1,2,3,6-tetrahydropyridine trifluoroacetate (0.140 mmol) and N,N-diisopropylethylamine (0.127 g, 0.171 mL, 0.980 mmol) in N,N dimethylformamide (2 mL) in a scintillation vial at 23° C. under N₂ was added tert-butyl N—[(Z)-{[(tert-butoxy)carbonyl]imino}(1H-pyrazol-1-yl)methyl]carbamate (0.130 g, 0.420 mmol). The reaction was stirred at 23° C. for 18 hours. The mixture was partitioned between ethyl acetate (130 mL) and saturated NH₄Cl solution (15 mL) and separated. The organic layer was washed with water (2×15 mL), saturated NaHCO₃ solution (15 mL), and brine (15 mL), then dried (Na₂SO₄), filtered, and concentrated. Silica gel chromatography (0 to 100% (10% isopropanol-90% ethyl acetate); 100 to 0% hexanes), followed by concentration and drying under vacuum, yielded the title compound (0.0809 g, 64%) as an off-white solid.

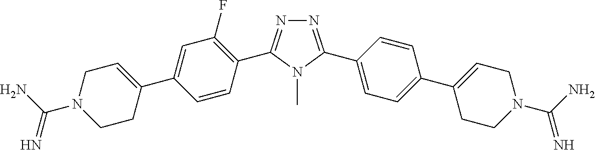 [SMILES: Cn1c(-c2ccc(C3=CCN(C(=N)N)CC3)cc2)nnc1-c1ccc(C2=CCN(C(=N)N)CC2)cc1F]

### Example 78

Synthesis of 4-(4-{5-[4-(1-carbamimidoyl-1,2,3,6-tetrahydropyridin-4-yl)-2-fluorophenyl]-4-methyl-4H-1,2,4-triazol-3-yl}phenyl)-1,2,3,6-tetrahydropyridine-1-carboximidamide trifluoroacetate: To a solution of tert-butyl N—[(Z)-(4-{4-[5-(4-{1-[(Z)-({[(tert-butoxy) carbonyl]amino}({[(tert-butoxy)carbonyl]imino})methyl]-1,2,3,6-tetrahydropyridin-4-yl}-2-fluorophenyl)-4-methyl-4H-1,2,4-triazol-3-yl]phenyl}-1,2,3,6-tetrahydropyridin-1-yl)({[(tert-butoxy)carbonyl]imino})methyl]carbamate (0.0770 g, 0.0856 mmol) and anisole (0.0925 g, 0.0932 mL, 0.856 mmol) in dichloromethane (2 mL) at 23° C. under N₂ was added trifluoroacetic acid (0.5 mL, 6.53 mmol) dropwise. After four hours, the mixture was concentrated, then suspended in CH₂Cl₂ and concentrated again, dissolved in 4:1 MeCN-water, frozen at −78° C., and lyophilized. The residue was purified by reverse-phase chromatography (SiliaSep C₁₈ 40 g column; 5 to 50% MeCN-water (both with 0.1% trifluoroacetic acid) over 23 min.). The fractions containing the title compound were combined, diluted with water, then lyophilized to yield the desired compound (0.0687 g, 80%) as a flocculent, white solid. ¹H NMR (300 MHz, DMSO-d₆) δ ppm 7.76-7.85 (m, 2H) 7.63-7.71 (m, 3H) 7.56-7.63 (m, 1H) 7.41-7.56 (m, 9H) 6.42-6.51 (m, 1H) 6.30-6.40 (m, 1H) 4.08-4.13 (m, 4H) 3.63 (br t, J=5.27 Hz, 4H) 3.58 (d, J=1.76 Hz, 3H) 2.60-2.68 (m, 4H). ¹⁹F NMR (282 MHz, DMSO-d₆) δ ppm −74.25 (s, 1F) −113.12-−11.2.98 (m, 1F). LC/MS (M+H)⁺=500.

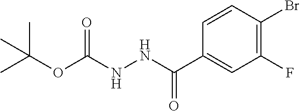 [SMILES: CC(C)(C)OC(=O)NNC(=O)c1ccc(Br)c(F)c1]

Synthesis of N′-(4-bromo-3-fluoro-benzoyl)-hydrazinecarboxylic acid tert-butyl ester: A mixture of 4-bromo-3-fluoro-benzoic acid (1.74 g, 7.94 mmol), N,N-diisopropylethylamine (3.91 g, 5.27 mL, 30.3 mmol), and 1-[bis(dimethyl amino)methylene]-1H-1,2,3-triazolo[4,5-b]pyridinium 3-oxid hexafluorophosphate (3.45 g, 9.08 mmol) in N,N dimethylformamide (18.9 mL) at 23° C. under N₂ was stirred for 20 minutes, before adding hydrazinecarboxylic acid tert-butyl ester (1.00 g, 7.57 mmol) and stirring at 23° C. for 18 hours and then heated at 50° C. for several hours. The mixture was concentrated to remove the N,N dimethylformamide, then partitioned between ethyl acetate (250 mL) and saturated NH₄Cl solution (25 mL) and separated. The organic layer was washed twice with water (2×25 mL), saturated NaHCO₃ solution (25 mL), then brine (25 mL), then dried (Na₂SO₄), filtered, and concentrated. Silica gel chromatography (0 to 40% ethyl acetate-hexanes), followed by concentration and drying under vacuum, yielded the title compound (1.3847 g, 55%) as a tan solid.

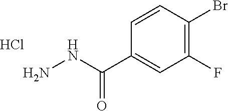 [SMILES: Cl ; NNC(=O)c1ccc(Br)c(F)c1]

Synthesis of 4-bromo-3-fluoro-benzoic acid hydrazide hydrochloride: To a solution of N′-(4-bromo-3-fluoro-benzoyl)-hydrazinecarboxylic acid tert-butyl ester (1.38 g, 4.14 mmol) in dichloromethane (21 mL) at 23° C. under N₂ was added 4 N HCl-1,4-dioxane solution (10.4 mL, 41.4 mmol) dropwise. The reaction was stirred for 18 hours. The mixture was concentrated, suspended in CH₂Cl₂ and concentrated again, then dried under vacuum to yield the title compound (1.1017 g, 99%) as a tan solid.

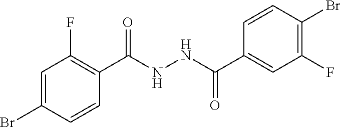 [SMILES: O=C(NNC(=O)c1ccc(Br)cc1F)c1ccc(Br)c(F)c1]

Synthesis of 4-bromo-3-fluoro-benzoic acid N′-(4-bromo-2-fluoro-benzoyl)-hydrazide: To a suspension of 4-bromo-3-fluoro-benzoic acid hydrazide hydrochloride (1.10 g, 4.08 mmol) and N,N-diisopropylethylamine (1.32 g, 1.78 mL, 10.2 mmol) in CHCl₃ (20 mL) at 0° C. under. N₂ was added a solution of 4-bromo-2-fluoro-benzoyl chloride (0.969 g, 4.08 mmol) in CHCl₃ (20 mL) dropwise via an addition funnel over 30 minutes. The reaction was warmed to 23° C. and stirred for 72 hours. The mixture was concentrated. The residue was diluted with water (30 mL), sonicated to disperse the gummy solid, then stirred for 60 minutes with occasional sonication. The water was decanted from the gummy solid, and MeOH (20 mL) was added. After brief sonication and stirring for 15 minutes, the solid was collected by filtration and washed with a small amount of additional MeOH, then ether, then dried under vacuum to yield the title compound (1.479 g, 84%) as an off-white solid.

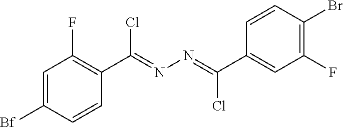

Synthesis of (Z)-4-bromo-N—[(Z)-(4-bromo-2-fluorophenyl)(chloro)methylidene]-3-fluorobenzene-1-carbohydrazonoyl chloride: To a suspension of 4-bromo-3-fluoro-benzoic acid N′-(4-bromo-2-fluoro-benzoyl)-hydrazide (0.300 g, 0.691 mmol) in anhydrous toluene (3.5 mL) in a pressure tube at 23° C. under N₂ was gradually added PCl₅ (0.432 g, 2.07 mmol). The mixture was heated at 95° C. for 18 hours. The reaction was cooled to 23° C. and concentrated, suspended in toluene and concentrated again, then dried under vacuum to yield the title compound (0.3185 g, 98%) as a light yellow solid, which was used without purification.

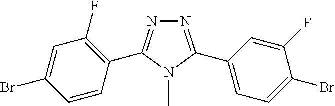 [SMILES: Cn1c(-c2ccc(Br)c(F)c2)nnc1-c1ccc(Br)cc1F]

Synthesis of 3-(4-bromo-2-fluoro-phenyl)-5-(4-bromo-3-fluoro-phenyl)-4-methyl-4H-[1,2,4]triazole: To a solution of (Z)-4-bromo-N—[(Z)-(4-bromo-2-fluorophenyl)(chloro)methylidene]-3-fluorobenzene-1-carbohydrazonoyl chloride (0.691 mmol) in anhydrous toluene (4 mL) at 0° C. under N₂ was added methylamine solution (33% in EtOH; 0.860 mL, 6.91 mmol) dropwise. After 60 minutes, the reaction was permitted to warm gradually to 23° C. and stirred for 18 hours. Acetic acid (1 mL) was added, and the mixture was heated to 60° C. for one hour before cooling to 23° C. and concentrating. The residue was partitioned between ethyl acetate (130 mL) and saturated NaHCO₃ solution (30 mL) and separated. The organic layer was washed with saturated Na₂CO₃ solution (15 mL), water (15 mL), and brine (15 mL), then dried (Na₂SO₄), filtered, and concentrated. Silica gel chromatography (0 to 100% (10% isopropanol-90% ethyl acetate); 100 to 0% hexanes), followed by concentration and drying under vacuum, yielded the title compound (0.2195 g, 74%) as a white solid.

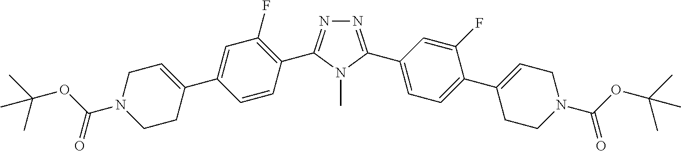 [SMILES: Cn1c(-c2ccc(C3=CCN(C(=O)OC(C)(C)C)CC3)c(F)c2)nnc1-c1ccc(C2=CCN(C(=O)OC(C)(C)C)CC2)cc1F]

Synthesis of tert-butyl 4-{4-[5-(4-{1-[(tert-butoxy)carbonyl]-1,2,3,6-tetrahydro pyridin-4-yl}-2-fluorophenyl)-4-methyl-4H-1,2,4-triazol-3-yl]-2-fluorophenyl}-1,2,3,6-tetrahydropyridine-1-carboxylate: A suspension of 3-(4-bromo-2-fluoro-phenyl)-5-(4-bromo-3-fluoro-phenyl)-4-methyl-4H-[1,2,4]triazole (0.100 g, 0.233 mmol) in 1,4-dioxane (2.5 mL) was degassed by bubbling nitrogen through the mixture for several minutes. After adding 2 M Na₂CO₃ solution (0.583 mL, 1.17 mmol), tert-butyl 5,6-dihydro-4-(4,4,5,5-tetramethyl-1,3,2-dioxaborolan-2-yl)pyridine-1(2H)-carboxylate (0.151 g, 0.489 mmol), and 1,1′-bis(diphenylphosphino)ferrocene]dichloropalladium(II), CH₂Cl₂ adduct (0.0190 g, 0.0233 mmol), the reaction was degassed for two more minutes, then heated at 80° C. for 18 hours. The mixture was cooled to 23° C. and partitioned between CH₂Cl₂ (80 mL) and saturated NaHCO₃ solution (15 mL) and separated. The aqueous layer was re-extracted with CH₂Cl₂ (2×20 mL). The organic layers were combined and washed with brine (15 mL), then dried (Na₂SO₄), filtered, and concentrated. Silica gel chromatography (0 to 100% (10% isopropanol-90% ethyl acetate); 100 to 0% hexanes), followed by concentration and drying under vacuum, yielded the desired compound (0.1221 g, 83%) as a tan solid.

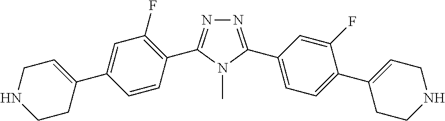 [SMILES: Cn1c(-c2ccc(C3=CCNCC3)c(F)c2)nnc1-c1ccc(C2=CCNCC2)cc1F]

Synthesis of 4-(2-fluoro-4-{5-[2-fluoro-4-(1,2,3,6-tetrahydropyridin-4-yl)phenyl]-4-methyl-4H-1,2,4-triazol-3-yl}phenyl)-1,2,3,6-tetrahydropyridine trifluoroacetate: To a solution of tert-butyl 4-{4-[5-(4-{1-[(tert-butoxy)carbonyl]-1,2,3,6-tetrahydropyridin-4-yl}-2-fluorophenyl)-4-methyl-4H-1,2,4-triazol-3-yl]-2-fluorophenyl}-1,2,3,6-tetrahydropyridine-1-carboxylat e(0.119 g, 0.188 mmol) and anisole (0.203 g, 0.204 mL, 1.88 mmol) in dichloromethane (2 mL) at 23° C. under N₂ was added trifluoroacetic acid (0.5 mL, 6.53 mmol) dropwise. After three hours, the mixture was concentrated, then dissolved in MeCN (2 mL) and concentrated again. The residue was dissolved in 4:1 MeCN-water, frozen at −78° C., then lyophilized to yield the title compound (0.1404 g, quantitative) as a flocculent, tan solid.

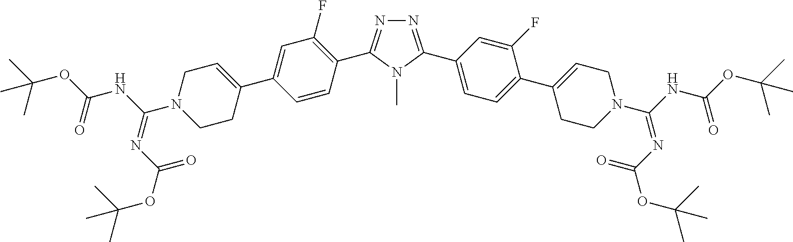 [SMILES: Cn1c(-c2ccc(C3=CCN(/C(=N\C(=O)OC(C)(C)C)NC(=O)OC(C)(C)C)CC3)c(F)c2)nnc1-c1ccc(C2=CCN(/C(=N\C(=O)OC(C)(C)C)NC(=O)OC(C)(C)C)CC2)cc1F]

Synthesis of tert-butyl N—[(Z)-(4-{4-[5-(4-{1-[(Z)-{[(tert-butoxy)carbonyl]amino}({[(tert-butoxy)carbonyl]imino})methyl]-1,2,3,6-tetrahydropyridin-4-yl}-2-fluorophenyl)-4-methyl-4H-1,2,4-triazol-3-yl]-2-fluorophenyl}-1,2,3,6-tetrahydropyridin-1-yl)({[(tert-butoxy)carbonyl]imino})methyl]carbamate: To a solution of 4-(2-fluoro-4-{5-[2-fluoro-4-(1,2,3,6-tetrahydropyridin-4-yl) phenyl]-4-methyl-4H-1,2,4-triazol-3-yl}phenyl)-1,2,3,6-tetrahydropyridine trifluoroacetate (0.188 mmol) and N,N-diisopropylethylamine (0.170 g, 0.229 mL, 1.32 mmol) in N,N dimethylformamide (2 mL) in a scintillation vial at 23° C. under N₂ was added tert-butyl N—[(Z)-{[(tert-butoxy)carbonyl]imino}(1H-pyrazol-1-yl)methyl]carbamate (0.175 g, 0.564 mmol). The reaction was stirred at 23° C. for 18 hours. The mixture was partitioned between ethyl acetate (130 mL) and saturated NH₄Cl solution (15 mL) and separated. The organic layer was washed with 5% KHSO₄ solution (15 mL), water (15 mL), saturated NaHCO₃ solution (15 mL), and brine (15 mL), then dried (Na₂SO₄), filtered, and concentrated. Silica gel chromatography (0 to 100% (10% isopropanol 90% ethyl acetate); 100 to 0% hexanes), followed by concentration and drying under vacuum, yielded the title compound (0.1108 g, 64%) as an off-white solid.

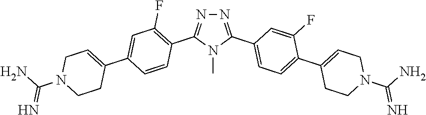 [SMILES: Cn1c(-c2ccc(C3=CCN(C(=N)N)CC3)c(F)c2)nnc1-c1ccc(C2=CCN(C(=N)N)CC2)cc1F]

### Example 79

Synthesis of 4-(4-{5-[4-(1-carbamimidoyl-1,2,3,6-tetrahydropyridin-4-yl)-2-fluorophenyl]-4-methyl-4H-1,2,4-triazol-3-yl}-2-fluorophenyl)-1,2,3,6-tetrahydro pyridine-1-carboximidamide trifluoroacetate: To a solution of tert-butyl N—[(Z)-(4-{4-[5-(4-{1-[(Z)-{[(tert-butoxy)carbonyl]amino}({[(tert-butoxy)carbonyl]imino})methyl]-1,2,3,6-tetrahydropyridin-4-yl}-2-fluorophenyl)-4-methyl-4H-1,2,4-triazol-3-yl]-2-fluorophenyl}-1,2,3,6=tetrahydropyridin-1-yl)({[(tert-butoxy)carbonyl]imino})methyl]carbamate (0.108 g, 0.118 mmol) and anisole (0.0636 g, 0.0641 mL, 0.588 mmol) in dichloromethane (2 mL) in a scintillation vial at 23° C. under. N₂ was added trifluoroacetic acid (0.5 mL, 6.53 mmol) dropwise. After four hours, the mixture was concentrated, then dissolved in MeCN (2 mL) and concentrated again, dissolved in 4:1 MeCN water, frozen at −78° C., and lyophilized to yield the title compound (0.0887 g, 100%) as an off-white solid. ¹H NMR (300 MHz, DMSO-d₆) δ ppm 7.41-7.71 (m, 15H) 6.38-6.52 (m, 1H) 6.08-6.22 (m, 1H) 4.04-4.15 (m, 4H) 3.58-3.63 (m, 7H) 2.6.1 (m, J=1.80 Hz, 4H). ¹⁹F NMR. (282 MHz, DMSO-d₆) δ ppm −73.93 (s, 1F)-113.05-−112.89 (m, 1F)-114.33-−114.14 (m, 1F). LC/MS (M+H)⁺=518.

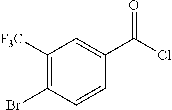 [SMILES: O=C(Cl)c1ccc(Br)c(C(F)(F)F)c1]

Synthesis of 4-bromo-3-trifluoromethyl-benzoyl chloride: To a suspension of 4-Bromo-3-trifluoromethyl-benzoic acid (2.500 g, 9.32 mmol) and N,N dimethylformamide (0.020 mL) in CHCl₃ (25 mL) at 23° C. under N₂ was added oxalyl chloride (1.780 g, 1.20 mL, 14.0 mmol) dropwise over 20 minutes. After three hours, the reaction was concentrated. The residue was used without purification.

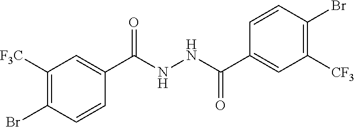 [SMILES: O=C(NNC(=O)c1ccc(Br)c(C(F)(F)F)c1)c1ccc(Br)c(C(F)(F)F)c1]

Synthesis of 4-bromo-3-trifluoromethyl-benzoic acid N-(4-bromo-3-trifluoromethyl-benzoyl)-hydrazide: To a solution of hydrazine hydrate (0.224 g, 4.47 mmol) and N,N-diisopropylethylamine (1.560 g, 2.30 mL, 12.1 mmol) in CHCl₃ (20 mL) at 0° C. under N₂ was added a solution of 4-bromo-3-trifluoromethyl-benzoyl chloride (9.32 mmol) in CHCl₃ (10 mL) dropwise over 10 minutes. The cooling bath warmed to 23° C. and the reaction was stirred for 72 hours. The mixture was concentrated. The residue was suspended in water (20 mL), then stirred at 23° C. for three hours. The water was decanted, and the residue was triturated with MeOH (10 mL), which was also decanted. Drying under vacuum yielded the title compound, which was used without further purification.

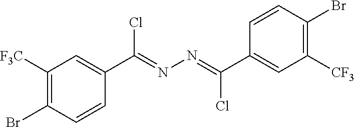 [SMILES: FC(F)(F)c1cc(/C(Cl)=N/N=C(\Cl)c2ccc(Br)c(C(F)(F)F)c2)ccc1Br]

Synthesis of (Z)-4-bromo-N—[(Z)-[4-bromo-3-(trifluoromethyl)phenyl](chloro) methylidene]-3-(trifluoromethyl)benzene-1-carbohydrazonoyl chloride: To a suspension of 4-bromo-3-trifluoromethyl-benzoic acid N′-(4-bromo-3-trifluoromethyl-benzoyl)-hydrazide (1.860 g, 3.50 mmol) in toluene (15 mL) at 23° C. under N₂ was added PCl₅ (2.200 g, 10.5 mmol). The mixture was heated at 60° C. for one hour, then 100° C. for 18 hours. The reaction was cooled to 23° C. and concentrated, then quenched by adding ice and water. The water was decanted; the residue was rinsed with MeOH (5 mL), which was also decanted. Acetonitrile (30 mL) was added, and the residue was concentrated and dried under vacuum to yield the title compound (2.500 g, quantitative), which was used without purification.

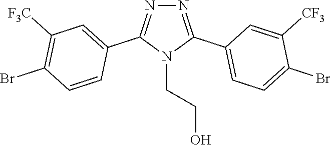 [SMILES: OCCn1c(-c2ccc(Br)c(C(F)(F)F)c2)nnc1-c1ccc(Br)c(C(F)(F)F)c1]

Synthesis of 2-[3,5-bis-(4-bromo-3-trifluoromethyl-phenyl)-[1,2,4]triazol-4-yl]-ethanol: To a solution of (Z)-4-bromo-N—[(Z)-[4-bromo-3-(trifluoromethyl)phenyl](chloro) methylidene]-3-(trifluoromethyl)benzene-1-carbohydrazonoyl chloride (1.400 g, 2.45 mmol) in MeOH (8 mL) was added ethanolamine (0.344 g, 5.63 mmol) dropwise over two minutes, followed five minutes later by INN-diisopropylethylamine (0.948 g, 1.35 mL, 8.35 mmol) dropwise over two minutes. After stirring for 18 hours at 23° C., acetic acid (4 mL) was added, and the reaction was heated at 100° C. for three hours. The reaction was cooled to 23° C. and concentrated. Water. (20 mL) was added, and the resulting solid was removed by filtration 30 minutes later. This solid was washed with ethyl acetate (25 mL), which was dried, concentrated, and dried under vacuum to yield the title product (0.990 g, 72%) as a brown solid.

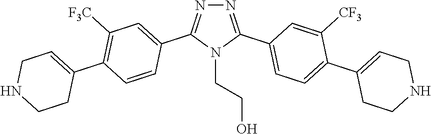 [SMILES: OCCn1c(-c2ccc(C3=CCNCC3)c(C(F)(F)F)c2)nnc1-c1ccc(C2=CCNCC2)c(C(F)(F)F)c1]

Synthesis of 2-{3,5-bis-[4-(1,2,3,6-tetrahydro-pyridin-4-yl)-3-trifluoromethyl-phenyl]-[1,2,4]triazol-4-yl}-ethanol trifluoroacetate: The title compound was prepared according to the procedure of 4-(2-Methyl-4-{5-methyl-4-[4-(1,2,3,6-tetrahydropyridin-4-yl)phenyl]-1H-1,2,3-triazol-1-yl}phenyl)-1,2,3,6-tetrahydropyridine except that 1-(4-bromo-3-methylphenyl)-4-(4-bromophenyl)-5-methyl-1H-1,2,3-triazole was replaced with 2-[3,5-bis-(4-bromo-3-trifluoromethyl-phenyl)-[1,2,4]triazol-4-yl]-ethanol.

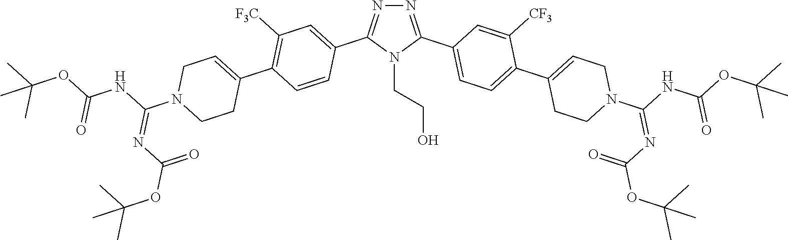 [SMILES: CC(C)(C)OC(=O)/N=C(/NC(=O)OC(C)(C)C)N1CC=C(c2ccc(-c3nnc(-c4ccc(C5=CCN(/C(=N\C(=O)OC(C)(C)C)NC(=O)OC(C)(C)C)CC5)c(C(F)(F)F)c4)n3CCO)cc2C(F)(F)F)CC1]

Synthesis of N—[(Z)-(4-{4-[5-(4-{1-[(Z)-{[(tert-butoxy)carbonyl]amino}({[(tert-butoxy)carbonyl]imino})methyl]-1,2,3,6-tetrahydropyridin-4-yl}-3-(trifluoromethyl)phenyl)-4-(2-hydroxyethyl)-4H-1,2,4-triazol-3-yl]-2-(trifluoromethyl)phenyl}-1,2,3,6-tetrahydropyridin-1-yl)({[(tert-butoxy)carbonyl]imino})methyl]carbamate: The title compound was prepared according to the procedure of tert-butyl N—[(Z)-(4-{4-[5-(4-{1-[(Z)-{[(tert-butoxy)carbonyl]amino}({[(tert-butoxy)carbonyl]imino})methyl]-1,2,3,6-tetrahydropyridin-4-yl}-2-fluorophenyl)-4-methyl-4H-1,2,4-triazol-3-yl]-2-fluorophenyl}-1,2,3,6-tetrahydropyridin-1-yl)({[(tert-butoxy)carbonyl]imino})methyl]carbamate except that 4-(2-fluoro-4-{5-[2-fluoro-4-(1,2,3,6-tetrahydropyridin-4-yl) phenyl]-4-methyl-4H-1,2,4-triazol-3-yl}phenyl)-1,2,3,6-tetrahydropyridine trifluoroacetate was replaced with 2-{3,5-bis-[4-(1,2,3,6-tetrahydro-pyridin-4-yl)-3-trifluoromethyl-phenyl]-[1,2,4]triazol-4-yl}-ethanol trifluoroacetate.

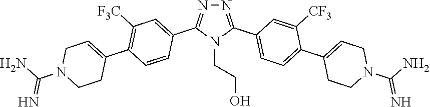 [SMILES: N=C(N)N1CC=C(c2ccc(-c3nnc(-c4ccc(C5=CCN(C(=N)N)CC5)c(C(F)(F)F)c4)n3CCO)cc2C(F)(F)F)CC1]

### Example 80

Synthesis of 4-(4-{5-[4-(1-carbamimidoyl-1,2,3,6-tetrahydropyridin-4-yl)-3-(trifluoromethyl)phenyl]-4-(2-hydroxyethyl)-4H-1,2,4-triazol-3-yl}-2-(trifluoromethyl)phenyl)-1,2,3,6-tetrahydropyridine-1-carboximidamide hydrochloride: To a solution of tert-butyl N—[(Z)-(4-{4-[5-(4-{1-[(Z)-{[(tert-butoxy)carbonyl]amino}({[(tert-butoxy)carbonyl]imino})methyl]-1,2,3,6-tetrahydropyridin-4-yl}-3-(trifluoromethyl)phenyl)-4-(2-hydroxyethyl)-4H-1,2,4-triazol-3-yl]-2-(trifluoromethyl)phenyl}-1,2,3,6-tetrahydropyridin-1-yl)({[(tert-butoxy)carbonyl]imino})methyl]carbamate (0.134 g, 0.128 mmol) and anisole (0.0691 g, 0.0696 mL, 0.639 mmol) in dichloromethane (2 mL) at 23° C. under N₂ was added 4 N HCl-1,4-dioxane solution (0.639 mL, 2.56 mmol) dropwise. After adding additional CH₂Cl₂ (2 mL), the reaction was stirred at 23° C. for 18 hours. Additional HCl-1,4-dioxane solution (0.320 mL) was added dropwise, and the reaction was stirred for three hours before concentrating (without heating). The residue was suspended in MeCN and concentrated again, then dried under vacuum to yield the title compound (0.1012 g, quantitative) as an off-white solid dbw6-024-a: ¹H NMR (300 MHz, DMSO-d₆) δ ppm 8.13 (d, J=1.17 Hz, 2H) 8.00-8.09 (m, 2H) 7.52-7.67 (m, 10H) 5.68-5.79 (m, 2H) 4.21 (m, J=4.10 Hz, 2H) 4.05 (m, J=1.80 Hz, 4H) 3.61-3.65 (m, 4H) 3.27 (t, J=5.27 Hz, 2H) 2.43-2.46 (m, 4H). ¹⁹F NMR (282 MHz, DMSO-d₆) δ ppm −56.92 (s, 1F). LC/MS (M+H)⁺=648.

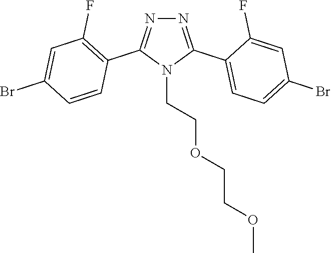 [SMILES: COCCOCCn1c(-c2ccc(Br)cc2F)nnc1-c1ccc(Br)cc1F]

Synthesis of 3,5-bis-(4-bromo-2-fluoro-phenyl)-4-[2-(2-methoxy-ethoxy)-ethyl]-4H-[1,2,4]triazole: The title compound was prepared according to the procedure of 3,5-bis-(4-bromo-phenyl)-4-(2-methoxy-ethyl)-4H-[1,2,4]triazole except that (Z)-4-bromo-N—[(Z)-(4-bromophenyl) (chloro)methylidene]benzene-1-carbohydrazonoyl chloride was replaced with (Z)-4-bromo-N—[(Z)-(4-bromo-2-fluorophenyl)(chloro)methylidene]-2-fluorobenzene-1-carbohydrazonoyl chloride.

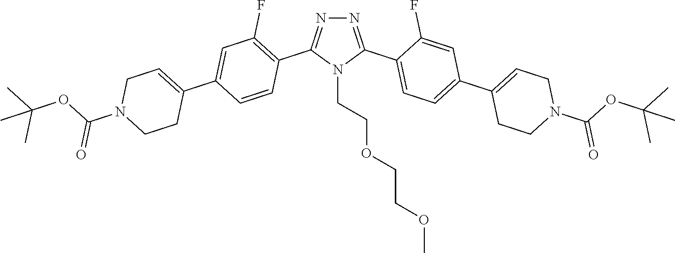 [SMILES: COCCOCCn1c(-c2ccc(C3=CCN(C(=O)OC(C)(C)C)CC3)cc2F)nnc1-c1ccc(C2=CCN(C(=O)OC(C)(C)C)CC2)cc1F]

Synthesis of tert-butyl 4-{4-[5-(4-{1-[(tert-butoxy)carbonyl]-1,2,3,6-tetrahydropyridin-4-yl}-2-fluorophenyl)-4-[2-(2-methoxyethoxy)ethyl]-4H-1,2,4-triazol-3-yl]-3-fluorophenyl}-1,2,3,6-tetrahydropyridine-1-carboxylate: A suspension of 3,5-bis-(4-bromo-2-fluoro-phenyl)-4-[2-(2-methoxy-ethoxy)-ethyl]-4H-[1,2,4]triazole (0.125 g, 0.242 mmol) in 1,4-dioxane (2.5 mL) was degassed by bubbling nitrogen through the mixture for 5 minutes. After adding 2 M Na₂CO₃ solution (0.604 mL, 1.21 mmol), tert-butyl 5,6-dihydro-4-(4,4,5,5-tetramethyl-1,3,2-dioxaborolan-2-yl)pyridine-1(2H)-carboxylate (0.157 g, 0.508 mmol), and 1,1′-bis(diphenylphosphino)ferrocene] dichloropalladium(II), CH₂Cl₂ adduct (0.0197 g, 0.0242 mmol), the reaction was degassed for two more minutes, then heated at 80° C. for 18 hours. The mixture was cooled to 23° C. and partitioned between CH₂Cl₂ (80 mL) and saturated NaHCO₃ solution (15 mL) and separated. The aqueous layer was re-extracted with CH₂Cl₂ (2×20 mL). The organic layers were combined and washed with brine (0.15 mL), then dried (Na₂SO₄), filtered, and concentrated. Silica gel chromatography (0 to 100% (10% isopropanol 90% ethyl acetate); 100 to 0% hexanes), followed by concentration and drying under vacuum-yielded the title compound (0.1273 g, 73%).

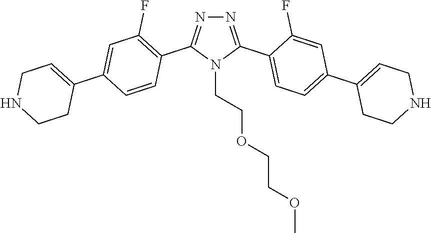 [SMILES: COCCOCCn1c(-c2ccc(C3=CCNCC3)cc2F)nnc1-c1ccc(C2=CCNCC2)cc1F]

Synthesis of 4-(3-fluoro-4-{5-[2-fluoro-4-(1,2,3,6-tetrahydropyridin-4-yl)phenyl]-4-[2-(2-methoxyethoxy)ethyl]-4H-1,2,4-triazol-3-yl}phenyl)-1,2,3,6-tetrahydropyridine trifluoroacetate: To a solution of tert-butyl 4-{4-[5-(4-{1-[(tert-butoxy)carbonyl]-1,2,3,6-tetrahydropyridin-4-yl}-2-fluorophenyl)-4-[2-(2-methoxyethoxy)ethyl]-4H-1,2,4-triazol-3-yl]-3-fluorophenyl}-1,2,3,6-tetrahydropyridine-1-carboxylate (0.124 g, 0.172 mmol) and anisole (0.0743 g, 0.0748 mL, 0.687 mmol) in dichloromethane (2 mL) at 23° C. under N₂ was added trifluoroacetic acid (0.5 mL, 6.53 mmol) dropwise. After two hours, the mixture was concentrated, then dissolved in MeCN (2 mL) and concentrated again, dissolved in 4:1 MeCN-water, frozen at −78° C., and lyophilized. The lyophilization was repeated to yield the title compound (0.1315 g, quantitative) as a flocculent, tan solid.

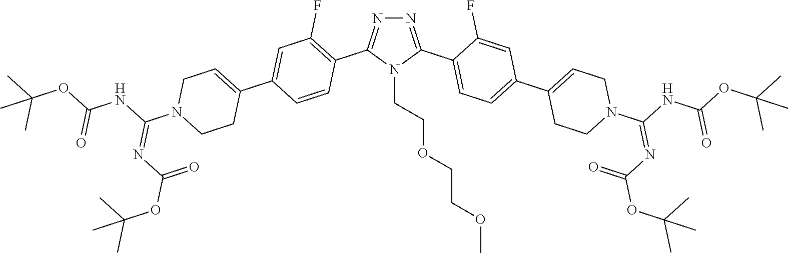 [SMILES: COCCOCCn1c(-c2ccc(C3=CCN(/C(=N\C(=O)OC(C)(C)C)NC(=O)OC(C)(C)C)CC3)cc2F)nnc1-c1ccc(C2=CCN(/C(=N\C(=O)OC(C)(C)C)NC(=O)OC(C)(C)C)CC2)cc1F]

Synthesis of tert-butyl N—[(Z)-(4-{4-[5-(4-{1-[(Z)-{[(tert-butoxy)carbonyl]amino}({[(tert-butoxy)carbonyl]imino})methyl]-1,2,3,6-tetrahydropyridin-4-yl}-2-fluorophenyl)-4-[2-(2-methoxyethoxy)ethyl]-4H-1,2,4-triazol-3-yl]-3-fluorophenyl}-1,2,3,6-tetrahydropyridin-1-yl)({[(tert-butoxy)carbonyl]imino})methyl]carbamate: To a solution of 4-(3-fluoro-4-{5-[2-fluoro-4-(1,2,3,6-tetrahydropyridin-4-yl)phenyl]-4-[2-(2-methoxyethoxy)ethyl]-4H-1,2,4-triazol-3-yl}phenyl)-1,2,3,6-tetrahydropyridine trifluoroacetate (0.124 g, 0.166 mmol) and N,N-diisopropylethylamine (0.150 g, 0.202 mL, 1.16 mmol) in N,N dimethylformamide (2 mL) at 23° C. under N₂ was added tert-butyl N—[(Z)-{[(tert-butoxy)carbonyl] imino}(1H-pyrazol-1-yl)methyl]carbonate (0.154 g, 0.497 mmol). The reaction was stirred at 23° C. for 72 hours. The mixture was partitioned between ethyl acetate (130 mL) and saturated NH₄Cl solution (20 mL) and separated (but did not separate well). The organic layer was washed again with saturated NH₄Cl solution (15 mL), saturated NaHCO₃ solution (15 mL), and brine (15 mL), then dried (Na₂SO₄), filtered, and concentrated. Silica gel chromatography (0 to 100% (15% isopropanol-85% ethyl acetate); 100 to 0% hexanes), followed by concentration and drying under vacuum, yielded the title compound (0.0612 g, 37%) as an off-white solid.

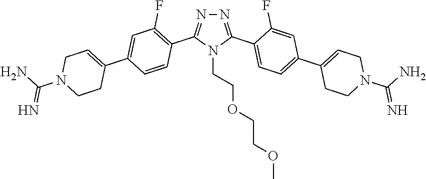 [SMILES: COCCOCCn1c(-c2ccc(C3=CCN(C(=N)N)CC3)cc2F)nnc1-c1ccc(C2=CCN(C(=N)N)CC2)cc1F]

### Example 81

Synthesis of 4-(4-{5-[4-(1-carbamimidoyl-1,2,3,6-tetrahydropyridin-4-yl)-2-fluorophenyl]-4-[2-(2-methoxyethoxy)ethyl]-4H-1,2,4-triazol-3-yl}-3-fluorophenyl)-1,2,3,6-tetrahydropyridine-1-carboximidamide: To a solution of tert-butyl N—[(Z)-(4-{4-[5-(4-{1-[(Z)-{[(tert-butoxy)carbonyl]amino}({[(tert-butoxy) carbonyl]imino})methyl]-1,2,3,6-tetrahydropyridin-4-yl}-2-fluorophenyl)-4-[2-(2-methoxyethoxy)ethyl]-4H-1,2,4-triazol-3-yl]-3-fluorophenyl}-1,2,3,6-tetrahydropyridin-1-yl)({[(tert-butoxy)carbonyl]imino})methyl]carbamate (0.0612 g, 0.0608 mmol) and anisole (0.0263 g, 0.0265 mL, 0.243 mmol) in dichloromethane (2 mL) at 23° C. under N₂ was added trifluoroacetic acid (0.5 mL, 6.53 mmol) dropwise. After four hours, the mixture was concentrated, then dissolved in MeCN (2 mL) and concentrated again (2×), dissolved in 4:1 MeCN-water, frozen at −78° C., and lyophilized to yield the title compound (0.0681 g, quantitative) as a flocculent, off-white solid. ¹H NMR (300 MHz, DMSO-d₆) δ ppm 7.62-7.71 (m, 2H) 7.58 (dd, J=12.02, 0.1.47 Hz, 2H) 7.43-7.53 (m, 10H) 6.39-6.53 (m, 2H) 4.06-4.15 (m, 4H) 4.01 (br t; J=4.40 Hz, 2H) 3.62 (br t, J=−5.57 Hz, 4H) 3.18 (t, J=5.27 Hz, 2H) 3.10 (s, 4H) 3.05 (s, 3H) 2.59-2.67 (m, 4H). ¹⁹F NMR. (282 MHz, DMSO-d₆) δ ppm −73.96 (s, 1F) −113.88-−113.75 (m, 1F). LC/MS (M+H)⁺=607.

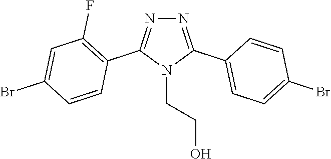 [SMILES: OCCn1c(-c2ccc(Br)cc2)nnc1-c1ccc(Br)cc1F]

Synthesis of 2-[3-(4-bromo-2-fluoro-phenyl)-5-(4-bromo-phenyl)-[1,2,4]triazol-4-yl]-ethanol: To a solution of (Z)-4-bromo-N—[(Z)-(4-bromo-2-fluorophenyl)(chloro)methylidene]benzene-1-carbohydrazonoyl chloride (0.424 g, 0.936 mmol) in anhydrous toluene (4.7 mL) at 0° C. under N₂ was added 2-amino-ethanol (0.572 g, 0.563 mL, 9.36 mmol) dropwise. The reaction was permitted to warm to 23° C. and stirred for 72 hours. Acetic acid (1 mL) was added, and the mixture was heated to 60° C. for 90 minutes then cooled to 23° C. and concentrating. The residue was partitioned between ethyl acetate (130 mL) and saturated NaHCO₃ solution (30 mL) and separated. The organic layer was washed with saturated NaHCO₃ solution (15 mL), water (15 mL), and brine (15 mL), then dried (Na₂SO₄), filtered, and concentrated. Silica gel chromatography (0 to 100% (10% isopropanol-90% ethyl acetate); 100 to 0% hexanes), followed by concentration and drying under vacuum, to yield the title compound (0.3057 g, 74%) as an off-white foam.

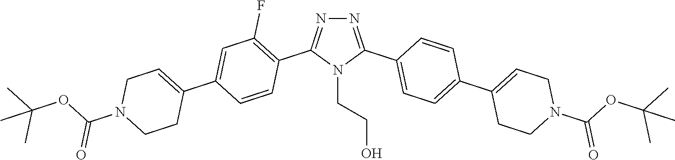 [SMILES: CC(C)(C)OC(=O)N1CC=C(c2ccc(-c3nnc(-c4ccc(C5=CCN(C(=O)OC(C)(C)C)CC5)cc4F)n3CCO)cc2)CC1]

Synthesis of tert-butyl 4-{4-[5-(4-{1-[(tert-butoxy)carbonyl]-1,2,3,6-tetrahydropyridin-4-yl}-2-fluorophenyl)-4-(2-hydroxyethyl)-4H-1,2,4-triazol-3-yl]phenyl}-1,2,3,6-tetrahydropyridine-1-carboxylate: A solution of tert-butyl 5,6-dihydro-4-(4,4,5,5-tetramethyl-1,3,2-dioxaborolan-2-yl)pyridine-1(2H)-carboxylate (0.184 g, 0.595 mmol) in 1,4-dioxane (2.5 mL) was degassed by bubbling nitrogen through the mixture for 5 minutes. After adding 2 M Na₂CO₃ solution (0.708 mL, 1.42 mmol), 2-[3-(4-bromo-2-fluoro-phenyl)-5-(4-bromo-phenyl)-[1,2,4]triazol-4-yl]-ethanol (0.125 g, 0.283 mmol), and 1,1′-bis(diphenylphosphino)ferrocene]dichloropalladium(II), CH₂Cl₂ adduct (0.0231 g, 0.0283 mmol), the reaction was degassed for two more minutes, heated at 80° C. for seven hours, then stirred at 23° C. for 72 hours. The mixture was partitioned between CH₂Cl₂ (80 mL) and saturated NaHCO₃ solution (20 mL) and separated. The aqueous layer was re-extracted with CH₂Cl₂ (2×20 mL). The organic layers were combined and washed with brine (15 mL), then dried (Na₂SO₄), filtered, and concentrated. Silica gel chromatography (0 to 100% (10% isopropanol-90% ethyl acetate); 100 to 0% hexanes), followed by concentration and drying under vacuum, yielded the title compound (0.1500 g, 82%) as an off-white foam.

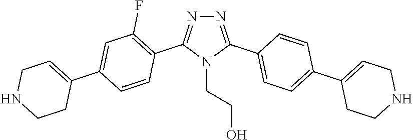 [SMILES: OCCn1c(-c2ccc(C3=CCNCC3)cc2)nnc1-c1ccc(C2=CCNCC2)cc1F]

Synthesis of 2-{3-[2-fluoro-4-(1,2,3,6-tetrahydro-pyridin-4-yl)-phenyl]-5-[4-(1,2,3,6-tetrahydro-pyridin-4-yl)-phenyl]-[1,2,4]triazol-4-yl}-ethanol hydrochloride: To a solution of tert-butyl 4-{4-[5-(4-{1-[(tert-butoxy)carbonyl]-1,2,3,6-tetrahydropyridin-4-yl}-2-fluorophenyl)-4-(2-hydroxyethyl)-4H-1,2,4-triazol-3-yl]phenyl}-1,2,3,6=tetrahydropyridine-1-carboxylate (0.146 g, 0.226 mmol) and anisole (0.122 g, 0.123 mL, 1.13 mmol) in dichloromethane (2.5 mL) at 23° C. under N₂ was added 4 N HCl-1,4-dioxane solution (2.00 mL, 8.00 mmol) dropwise. After two hours, the mixture was concentrated (without heating). The residue was suspended in MeCN (2 mL) and concentrated again (2×), then dried under vacuum to yield the title compound (0.1313 g, quantitative) as a light yellow solid.

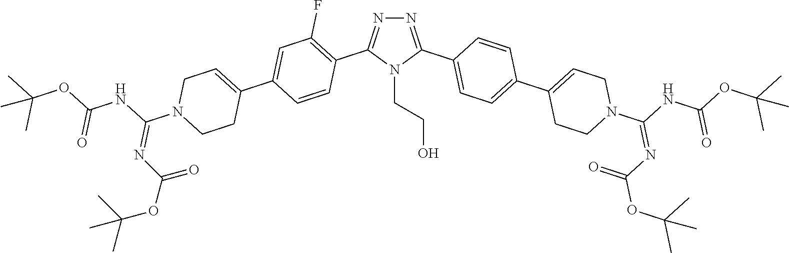 [SMILES: CC(C)(C)OC(=O)/N=C(/NC(=O)OC(C)(C)C)N1CC=C(c2ccc(-c3nnc(-c4ccc(C5=CCN(/C(=N\C(=O)OC(C)(C)C)NC(=O)OC(C)(C)C)CC5)cc4F)n3CCO)cc2)CC1]

Synthesis of tert-butyl N—[(Z)-(4-{4-[5-(4-{1-[(Z)-{[(tert-butoxy)carbonyl]amino}({[(tert-butoxy)carbonyl]imino})methyl]-1,2,3,6-tetrahydropyridin-4-yl}-2-fluorophenyl)-4-(2-hydroxyethyl)-4H-1,2,4-triazol-3-yl]phenyl}-1,2,3,6-tetrahydropyridin-1-yl)({[(tert-butoxy)carbonyl]imino})methyl]carbamate: To a solution of 2-{3-[2-fluoro-4-(1,2,3,6-tetrahydro-pyridin-4-yl)-phenyl]-5-[4-(1,2,3,6-tetrahydro-pyridin-4-yl)-phenyl]-[1,2,4]triazol-4-yl}-ethanol hydrochloride (0.226 mmol) and N,N-diisopropylethylamine (0.292 g, 0.394 mL, 2.26 mmol) in N,N dimethylformamide (2 mL) at 23° C. under N₂ was added tert-butyl N—[(Z)-{[(tert-butoxy)carbonyl]imino}(1H-pyrazol-1-yl) methyl]carbamate (0.210 g, 0.678 mmol). The reaction was stirred at 23° C. for 18 hours. The mixture was partitioned between ethyl acetate (130 mL) and saturated NH₄Cl solution (20 mL) and separated. The organic layer was washed again with saturated NH₄Cl solution (15 mL), saturated NaHCO₃ solution (15 mL), and brine (15 mL), then dried (Na₂SO₄), filtered, and concentrated. Silica gel chromatography (0 to 0.100% (10% isopropanol . . . 90% ethyl acetate); 100 to 0% hexanes), followed by concentration and drying under vacuum, yielded the title compound (0.1207 g, 57%) as an off-white solid.

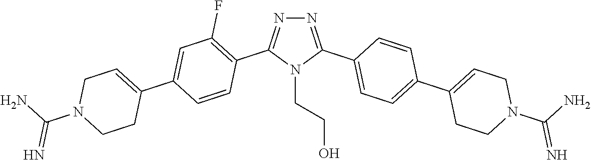 [SMILES: N=C(N)N1CC=C(c2ccc(-c3nnc(-c4ccc(C5=CCN(C(=N)N)CC5)cc4F)n3CCO)cc2)CC1]

### Example 82

Synthesis of 4-(4-{5-[4-(1-carbamimidoyl-1,2,3,6-tetrahydropyridin-4-yl)-2-fluorophenyl]-4-(2-hydroxyethyl)-4H-1,2,4-triazol-3-yl}phenyl)-1,2,3,6-tetrahydropyridine-1-carboximidamide hydrochloride: To a solution of tert-butyl N—[(Z)-(4-{4-[5-(4-{1-[(Z)-{[(tert-butoxy)carbonyl]amino}({[(tert-butoxy)carbonyl]imino})methyl]-1,2,3,6-tetrahydropyridin-4-yl}-2-fluorophenyl)-4-(2-hydroxyethyl)-4H-1,2,4-triazol-3-yl]phenyl}-1,2,3,6-tetrahydropyridin-1-yl)({[(tert-butoxy)carbonyl]imino})methyl]carbamate (0.117 g, 0.126 mmol) and anisole (0.0680 g, 0.0685 mL, 0.629 mmol) in dichloromethane (2.5 mL) at 23° C. under N₂ was added 4 N HCl-1,4-dioxane solution (2.00 mL, 8.00 mmol) dropwise. A precipitate began to form the vial was occasionally sonicated to keep it suspended. After three hours, the mixture was concentrated, suspended in MeCN (2 mL) and concentrated again (2×), then dried under vacuum to yield the title compound (0.0847 g, 94%) as an off-white solid. ¹H NMR (300 MHz, DMSO-d₆) δ ppm 7.76-7.83 (m, 2H) 7.50-7.70 (m, 14H) 6.42-6.52 (m, 1H) 6.31-6.41 (m, 1H) 4.05-4.15 (m, 6H) 3.21 (t, J=5.57 Hz, 2H) 2.63 (br s, 4H). ¹⁹F NMR (282 MHz, DMSO-d₆) δ ppm −113.31-113.20 (m, 1F). LC/MS (M+H)⁺=530.

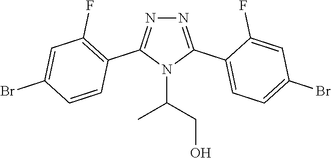 [SMILES: CC(CO)n1c(-c2ccc(Br)cc2F)nnc1-c1ccc(Br)cc1F]

dbw6-073-b:

Synthesis of 2-[3,5-bis-(4-bromo-2-fluoro-phenyl)-[1,2,4]triazol-4-yl]-propan-1-ol: To a solution of (Z)-4-bromo-N—[(Z)-(4-bromo-2-fluorophenyl)(chloro)methylidene]-2-fluorobenzene-1-carbohydrazonoyl chloride (0.789 mmol) in anhydrous toluene (5 mL) at 0° C. under N₂ was added a mixture (0.350 mL, each, 0.337 g, 4.49 mmol) of equal amounts of the D- and L-enantiomers of 2-amino-propan-1-ol dropwise, then rinsed the vial with anhydrous toluene (2×0.5 mL, also added to the reaction dropwise). The reaction was permitted to warm to 23° C. and stirred for 18 hours. Acetic acid (1 mL) was added, and the mixture was heated to 60° C. for three hours before cooling to 23° C. and concentrating. The residue was partitioned between ethyl acetate (130 mL) and saturated NaHCO₃ solution (35 mL) and separated. The organic layer was washed with water (15 mL), and brine (15 mL), then dried (Na₂SO₄), filtered, and concentrated. Silica gel chromatography (0 to 100% (10% isopropanol-90% ethyl acetate); 100 to 0% hexanes), followed by concentration and drying under vacuum, to yield the title compound (0.0857 g, 23%) as an off-white solid.

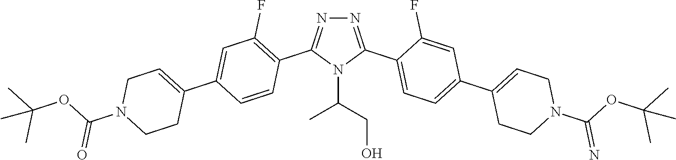 [SMILES: CC(CO)n1c(-c2ccc(C3=CCN(C(=N)OC(C)(C)C)CC3)cc2F)nnc1-c1ccc(C2=CCN(C(=O)OC(C)(C)C)CC2)cc1F]

Synthesis of tert-butyl 4-{4-[5-(4-{1-[(tert-butoxy)carbonyl]-1,2,3,6-tetrahydropyridin-4-yl}-2-fluorophenyl)-4-(1-hydroxypropan-2-yl)-4H-1,2,4-triazol-3-yl]-3-fluorophenyl}-1,2,3,6-tetrahydropyridine-1-carboxylate: A suspension of 2-[3,5-bis-(4-bromo-2-fluoro-phenyl)-[1,2,4]triazol-4-yl]-propan-1-ol (0.083 g, 0.175 mmol) in 1,4-dioxane (2.5 mL) in a scintillation vial was degassed by bubbling nitrogen through the mixture for several minutes. After adding 2 M Na₂CO₃ solution (0.439 mL, 0.877 mmol), tert-butyl 5,6-dihydro-4-(4,4,5,5-tetramethyl-1,3,2-dioxaborolan-2-yl)pyridine-1(2H)-carboxylate (0.114 g, 0.368 mmol), and 1,1′-bis(diphenylphosphino)ferrocene]dichloro palladium(II), CH₂Cl₂ adduct (0.0143 g, 0.0175 mmol), the reaction was degassed for two more minutes, heated at 80° C. for six hours, then stirred at 23° C. for 18 hours. The mixture was partitioned between CH₂Cl₂ (80 mL) and saturated NaHCO₃ solution (15 mL) and separated. The aqueous layer was re-extracted with CH₂Cl₂ (2×20 mL). The organic layers were combined and washed with brine (15 mL), then dried (Na₂SO₄), filtered, and concentrated. Silica gel chromatography (0 to 100% (10% isopropanol-90% ethyl acetate); 100 to 0% CH₂Cl₂), followed by concentration and drying under vacuum, yielded the title compound (0.0938 g, 79%) as an off-white solid.

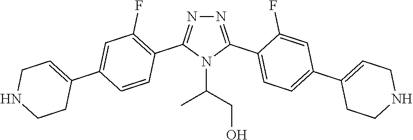 [SMILES: CC(CO)n1c(-c2ccc(C3=CCNCC3)cc2F)nnc1-c1ccc(C2=CCNCC2)cc1F]

Synthesis of 2-{3,5-bis-[2-fluoro-4-(1,2,3,6-tetrahydro-pyridin-4-yl)-phenyl]-[1,2,4]triazol-4-yl}-propan-1-ol hydrochloride: To a solution of tert-butyl 4-{4-[5-(4-{1-[(tert-butoxy)carbonyl]-1,2,3,6-tetrahydropyridin-4-yl}-2-fluorophenyl)-4-(1-hydroxypropan-2-yl)-4H-1,2,4-triazol-3-yl]-3-fluorophenyl}-1,2,3,6-tetrahydropyridine-1-carboxylate (0.0920 g, 0.136 mmol) and anisole (0.0734 g, 0.0739 mL, 0.679 mmol) in dichloromethane (2.5 mL) in a scintillation vial at 23° C. under N₂ was added 4 N HCl-1,4-dioxane solution (2.00 mL, 8.00 mmol) dropwise. A precipitate began to form. After stirring for 18 hours, the mixture was concentrated (without heating). The residue was suspended in MeCN (2 mL) and concentrated again (2×), then dried under vacuum to yield the title compound (0.0857 g, quantitative) as an off-white solid.

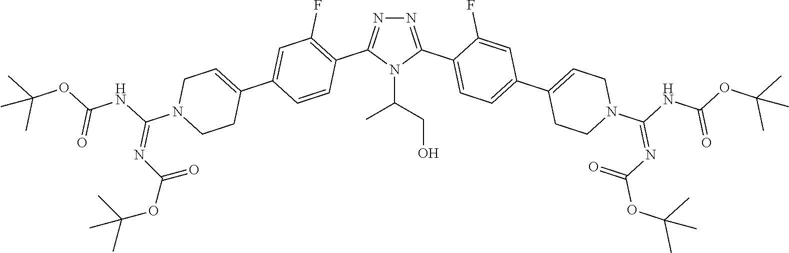 [SMILES: CC(CO)n1c(-c2ccc(C3=CCN(/C(=N\C(=O)OC(C)(C)C)NC(=O)OC(C)(C)C)CC3)cc2F)nnc1-c1ccc(C2=CCN(/C(=N\C(=O)OC(C)(C)C)NC(=O)OC(C)(C)C)CC2)cc1F]

Synthesis of tert-butyl N—[(Z)-(4-{4-[5-(4-{1-[(Z)-{[(tert-butoxy)carbonyl]amino}({[(tert-butoxy)carbonyl]imino})methyl]-1,2,3,6-tetrahydropyridin-4-yl}-2-fluorophenyl)-4-(1-hydroxypropan-2-yl)-4H-1,2,4-triazol-3-yl]-3-fluorophenyl}-1,2,3,6-tetrahydropyridin-1-yl)({[(tert-butoxy)carbonyl]imino})methyl]carbamate: To a solution of 2-{3,5-bis-[2-fluoro-4-(1,2,3,6-tetrahydro-pyridin-4-yl)-phenyl]-[1,2,4]triazol-4-yl}-propan-1-ol hydrochloride (0.136 mmol) and N,N-diisopropylethylamine (0.123 g, 0.166 mL, 0.952 mmol) in N,N dimethylformamide (3 mL) in a scintillation vial at 23° C. under N₂ was added tert-butyl N—[(Z)-{[(tert-butoxy)carbonyl]imino}(1H-pyrazol-1-yl)methyl]carbamate (0.127 g, 0.408 mmol). The reaction was stirred at 23° C. for 72 hours. LC-MS analysis showed some unreacted amine. The reaction was heated at 45° C. for 18 hours. The temperature was lowered to 40° C. and the suspension was again stirred for 18 hours. The mixture was partitioned between ethyl acetate (130 mL) and saturated NH₄Cl solution (20 mL) and separated. The organic layer was washed again with saturated NH₄Cl solution (15 mL), saturated NaHCO₃ solution (15 mL), and brine (15 mL), then dried (Na₂SO₄), filtered, and concentrated. Silica gel chromatography (0 to 80% (10% isopropanol-90% ethyl acetate); 100 to 20% CH₂Cl₂), followed by concentration and drying under vacuum, yielded the title compound (0.0535 g, 41%) as an off-white solid.

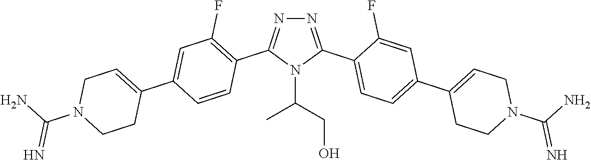 [SMILES: CC(CO)n1c(-c2ccc(C3=CCN(C(=N)N)CC3)cc2F)nnc1-c1ccc(C2=CCN(C(=N)N)CC2)cc1F]

### Example 83

4-(4-{5-[4-(1-carbamimidoyl-1,2,3,6-tetrahydropyridin-4-yl)-2-fluorophenyl]-4-(1-hydroxypropan-2-yl)-4H-1,2,4-triazol-3-yl}-3-fluorophenyl)-1,2,3,6-tetrahydropyridine-1-carboximidamide hydrochloride: To a solution of tert-butyl N—[(Z)-(4-{4-[5-(4-{1-[(Z)-{[(tert-butoxy)carbonyl]amino}({[(tert-butoxy)carbonyl]imino})methyl]-1,2,3,6-tetrahydropyridin-4-yl}-2-fluorophenyl)-4-(1-hydroxypropan-2-yl)-4H-1,2,4-triazol-3-yl]-3-fluorophenyl}-1,2,3,6-tetrahydropyridin-1-yl)({[(tert-butoxy)carbonyl]imino})methyl]carbamate (0.0535 g, 0.0556 mmol) and anisole (0.0301 g, 0.0303 mL, 0.278 mmol) in dichloromethane (3 mL) at 23° C. under N₂ was added 4 N HCl-1,4-dioxane solution (2.00 mL, 8.00 mmol) dropwise. After stirring for 18 hours, the mixture was concentrated, suspended in MeCN (2 mL) and concentrated again (2×), then dried under vacuum to yield the desired compound (0.0409 g, quantitative) as an off-white solid. ¹H NMR (300 MHz, DMSO-d₆) δ ppm 7.47-7.69 (m, 15H) 6.41-6.53 (m, 2H) 4.12 (br s, 4H) 3.99-4.08 (m, 1H) 3.64 (br t, J=4.98 Hz, 4H) 3.25 (br d, J=7.03 Hz, 2H) 2.64 (br s, 4H) 1.10 (d, J=7.03 Hz, 3H). ¹⁹F NMR (282 MHz, DMSO-d₆) δ ppm 112.49-−112.34 (m, 1F). LC/MS (M+H)⁺=562.

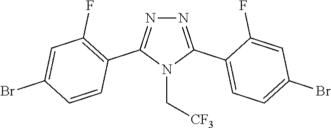 [SMILES: Fc1cc(Br)ccc1-c1nnc(-c2ccc(Br)cc2F)n1CC(F)(F)F]

Synthesis of 3,5-bis-(4-bromo-2-fluoro-phenyl)-4-(2,2,2-trifluoro-ethyl)-4H-[1,2,4]triazole: To a solution of (Z)-4-bromo-N—[(Z)-(4-bromo-2-fluorophenyl)(chloro)methylidene]-2-fluorobenzene-1-carbohydrazonoyl chloride (0.792 mmol) in anhydrous toluene (6 mL) at 0° C. under N₂ was added 2,2,2-trifluoro-ethylamine (0.784 g, 0.622 mL, 7.92 mmol) dropwise. The reaction warmed to 23° C. and was stirred for 18 hours. The mixture was heated to 60° C. for two hours. Acetic acid (1 mL) was added and the mixture was heated to 60° C. for four hours, then stirred at 23° C. for 18 hours. Additional acetic acid (1 mL) was added, and the reaction was heated at 80° C. for seven hours, then stirred at 23° C. for 18 hours. Additional 2,2,2-trifluoro-ethylamine (0.622 mL) was added, and the reaction was heated at 80° C. for 18 hours. The mixture was concentrated; the residue was suspended in 2,2,2-trifluoro-ethylamine (2 mL) and heated at 60° C. for four hours, then stirred at 23° C. for 18 hours. Acetic acid (3 mL) was added, and the reaction was heated at 60° C. for six hours, then stirred at 23° C. for 72 hours. The mixture was concentrated. The residue was partitioned between ethyl acetate (130 mL) and saturated NaHCO₃ solution (35 mL) and separated. The organic layer was washed with water (15 mL), and brine (15 mL), then dried (Na₂SO₄), filtered, and concentrated. Silica gel chromatography (0 to 60% ethyl acetate); 100 to 40% hexanes), followed by concentration and drying under vacuum, yielded the title compound (0.2698 g, 69%) as an off-white solid.

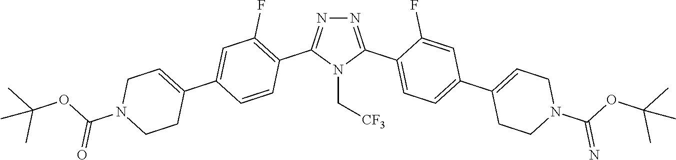 [SMILES: CC(C)(C)OC(=N)N1CC=C(c2ccc(-c3nnc(-c4ccc(C5=CCN(C(=O)OC(C)(C)C)CC5)cc4F)n3CC(F)(F)F)c(F)c2)CC1]

Synthesis of tert-butyl 4-{4-[5-(4-{1-[(tert-butoxy)carbonyl]-1,2,3,6-tetrahydropyridin-4-yl}-2-fluorophenyl)-4-(2,2,2-trifluoroethyl)-4H-1,2,4-triazol-3-yl]-3-fluorophenyl}-1,2,3,6-tetrahydropyridine-1-carboxylate: A solution of tert-butyl 5,6-dihydro-4-(4,4,5,5-tetramethyl-1,3,2-dioxaborolan-2-yl)pyridine-1(2H)-carboxylate (0.144 g, 0.465 mmol) in 1,4-dioxane (2.5 mL) in a scintillation vial was degassed by bubbling nitrogen through the mixture for several minutes. After adding 2 M Na₂CO₃ solution (0.553 mL, 1.1.1 mmol), 3,5-bis-(4-bromo-2-fluoro-phenyl)-4-(2,2,2-trifluoro-ethyl)-4H-[1,2,4]triazole (0.110 g, 0.221 mmol), and 1,1′-bis(diphenylphosphino) ferrocene]dichloropalladium(II), CH₂Cl₂ adduct (0.0181 g, 0.0221 mmol), the reaction was degassed for two more minutes, then heated at 80° C. for 18 hours. The mixture was partitioned between CH₂Cl₂ (80 mL) and saturated NaHCO₃ solution (20 mL) and separated. The aqueous layer was re-extracted with CH₂Cl₂ (2×20 mL). The organic layers were combined and washed with brine (15 mL), then dried (Na₂SO₄), filtered, and concentrated. Silica gel chromatography (0 to 100% ethyl acetate; 100 to 0% hexanes), followed by concentration and drying under vacuum, yielded the title compound (0.1358 g, 88%) as an off-white solid.

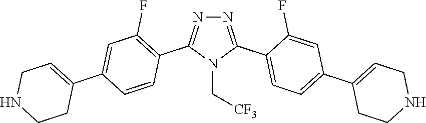 [SMILES: Fc1cc(C2=CCNCC2)ccc1-c1nnc(-c2ccc(C3=CCNCC3)cc2F)n1CC(F)(F)F]

Synthesis of 4-(3-fluoro-4-{5-[2-fluoro-4-(1,2,3,6-tetrahydropyridin-4-yl)phenyl]-4-(2,2,2-trifluoroethyl)-4H-1,2,4-triazol-3-yl}phenyl)-1,2,3,6-tetrahydropyridine hydrochloride: To a solution of tert-butyl 4-{4-[5-(4-{1-[(tert-butoxy)carbonyl]-1,2,3,6-tetrahydropyridin-4-yl}-2-fluoro phenyl)-4-(2,2,2-trifluoroethyl)-4H-1,2,4-triazol-3-yl]-3-fluorophenyl}-1,2,3,6-tetrahydro pyridine-1-carboxylate (0.1358 g, 0.194 mmol) and anisole (0.105 g, 0.105 mL, 0.968 mmol) in dichloromethane (3 mL) in a scintillation vial at 23° C. under N₂ was added 4 N HCl . . . 1,4-dioxane solution (2.00 mL, 8.00 mmol) dropwise. After stirring for 18 hours, the mixture was concentrated, suspended in MeCN (2 mL) and concentrated again (2×), then dried under vacuum to yield the title compound (0.1196 g, quantitative) as an off-white solid.

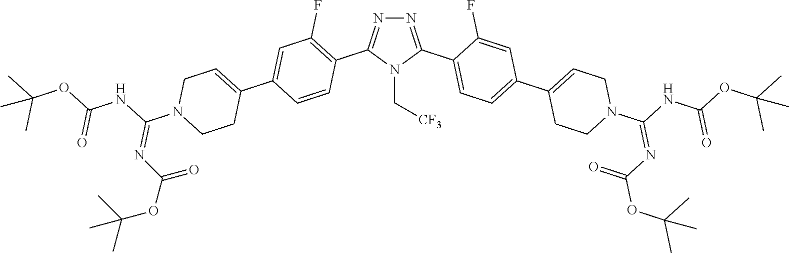 [SMILES: CC(C)(C)OC(=O)/N=C(/NC(=O)OC(C)(C)C)N1CC=C(c2ccc(-c3nnc(-c4ccc(C5=CCN(/C(=N\C(=O)OC(C)(C)C)NC(=O)OC(C)(C)C)CC5)cc4F)n3CC(F)(F)F)c(F)c2)CC1]

Synthesis of tert-butyl N—[(Z)-(4-{4-[5-(4-{1-[(Z)-{[(tert-butoxy)carbonyl]amino}({[(tert-butoxy)carbonyl]imino})methyl]-1,2,3,6-tetrahydropyridin-4-yl}-2-fluorophenyl)-4-(2,2,2-trifluoroethyl)-4H-1,2,4-triazol-3-yl]-3-fluorophenyl}-1,2,3,6-tetrahydropyridin-1-yl)({[(tert-butoxy)carbonyl]imino})methyl]carbamate: To a solution of 4-(3-fluoro-4-{5-[2-fluoro-4-(1,2,3,6-tetrahydropyridin-4-yl)phenyl]-4-(2,2,2-trifluoroethyl)-4H-1,2,4-triazol-3-yl}phenyl)-1,2,3,6-tetrahydropyridine hydrochloride (0.114 g, 0.187 mmol) and N,N-diisopropylethylamine (0.169 g, 0.228 mL, 1.31 mmol) in N,N dimethylformamide (3 mL) at 23° C. under N₂ was added tert-butyl N—[(Z)-{[tert-butoxy)carbonyl]imino}(1H-pyrazol-1-yl)methyl]carbamate (0.174 g, 0.560 mmol). The reaction was heated at 40° C. for 18 hours. The mixture was cooled to 23° C. and partitioned between ethyl acetate (130 mL) and saturated NH₄Cl solution (20 mL) and separated. The organic layer was washed again with saturated NH₄Cl solution (15 mL), saturated NaHCO₃ solution (15 mL), and brine (15 mL), then dried (Na₂SO₄), filtered, and concentrated. Silica gel chromatography (0 to 100% ethyl acetate; 100 to 0% hexanes), followed by concentration and drying under vacuum, yielded the title compound (0.1392 g, 76%) as an off-white solid.

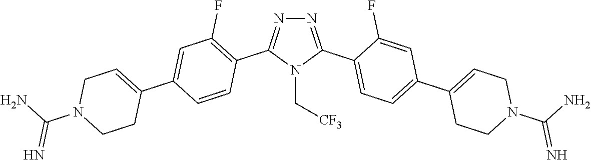 [SMILES: N=C(N)N1CC=C(c2ccc(-c3nnc(-c4ccc(C5=CCN(C(=N)N)CC5)cc4F)n3CC(F)(F)F)c(F)c2)CC1]

### Example 84

Synthesis of 4-(4-{5-[4-(1-carbamimidoyl-1,2,3,6-tetrahydropyridin-4-yl)-2-fluorophenyl]-4-(2,2,2-trifluoroethyl)-4H-1,2,4-triazol-3-yl}-3-fluorophenyl)-1,2,3,6-tetrahydropyridine-1-carboximidamide hydrochloride: To a solution of tert-butyl N—[(Z)-(4-{4-[5-(4-{1-[(Z)-{[(tert-butoxy)carbonyl]amino}({[(tert-butoxy)carbonyl]imino})methyl]-1,2,3,6-tetrahydropyridin-4-yl}-2-fluorophenyl)-4-(2,2,2-trifluoroethyl)-4H-1,2,4-triazol-3-yl]-3-fluorophenyl}-1,2,3,6-tetrahydropyridin-1-yl)({[(tert-butoxy)carbonyl]imino})methyl]carbamate (0.137 g, 0.139 mmol) and anisole (0.0751 g, 0.0757 mL, 0.695 mmol) in dichloromethane (3 mL) at 23° C. under N₂ was added 4 N HCl-1,4-dioxane solution (2.00 mL, 8.00 mmol) dropwise. After stirring for 72 hours, the mixture was concentrated, suspended in MeCN (2 mL) and concentrated again (2×), then dried under vacuum to yield the desired compound (0.0946 g, 98%) as an off-white solid. ¹H NMR (300 MHz, DMSO-d₆) δ ppm 7.66-7.73 (m, 2H) 7.52-7.65 (m, 12H) 6.44-6.56 (m, 2H) 4.86 (q, J=8.79 Hz, 2H) 4.13 (br s, 4H) 3.60-3.67 (m, 4H) 2.64 (br s, 4H). ¹⁹F NMR (282 MHz, DMSO-d₆) δ ppm −69.63 (br t, J=8.42 Hz, 1F)-113.92-−113.78 (m, 1F). LC/MS (M+H)⁺=586.

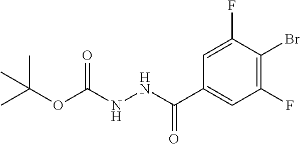 [SMILES: CC(C)(C)OC(=O)NNC(=O)c1cc(F)c(Br)c(F)c1]

Synthesis of N′-(4-bromo-3,5-difluoro-benzoyl)-hydrazinecarboxylic acid tert-butyl ester: To a mixture of 4-bromo-3,5-difluoro-benzoic acid (1.20 g, 5.07 mmol) in N,N dimethylformamide (13 mL) at 23° C. under N₂ was added N,N-diisopropylethylamine (2.62 g, 3.53 mL, 20.3 mmol) followed by 1-[bis(dimethylamino)methyl ene]-1H-1,2,3-triazolo[4,5-b]pyridinium 3-oxid hexafluorophosphate (2.31 g, 6.08 mmol). After 15 minutes, hydrazinecarboxylic acid tert-butyl ester (0.67 g, 5.07 mmol) was added, and the reaction was stirred at 23° C. for 72 hours. The reaction was heated at 50° C. for 18 hours. The mixture was concentrated to remove the N,N dimethylformamide, then partitioned between ethyl acetate (250 mL) and saturated NH₄Cl solution (25 mL) and separated. The organic layer was washed with 10% KHSO₄ solution (25 mL), water (25 mL), saturated NaHCO₃ solution (25 mL), and brine (25 mL), then dried (Na₂SO₄), filtered, and concentrated. Silica gel chromatography (0 to 40% ethyl acetate-hexanes), followed by concentration and drying under vacuum, yielded the title compound (0.7108 g, 40%) as an off-white solid.

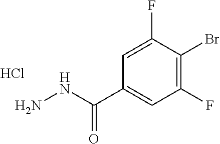 [SMILES: Cl ; NNC(=O)c1cc(F)c(Br)c(F)c1]

Synthesis of 4-bromo-3,5-difluoro-benzoic acid hydrazide hydrochloride: To a solution of N′-(4-bromo-3,5-difluoro-benzoyl)-hydrazinecarboxylic acid tert-butyl ester (0.710 g, 2.02 mmol) in dichloromethane (20 mL) 23° C. under N₂ was added 4 N HCl-1,4-dioxane solution (5.05 mL, 20.2 mmol) dropwise. The reaction was stirred for 18 hours. The mixture was concentrated, suspended in CH₂Cl₂ and concentrated again, then dried under vacuum to yield the title compound (0.5890 g, quantitative) as an off-white solid.

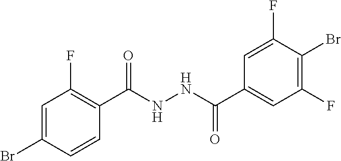 [SMILES: O=C(NNC(=O)c1ccc(Br)cc1F)c1cc(F)c(Br)c(F)c1]

Synthesis of 4-bromo-3,5-difluoro-benzoic acid N′-(4-bromo-2-fluoro-benzoyl)-hydrazide: To a suspension of 4-bromo-3,5-difluoro-benzoic acid hydrazide hydrochloride (0.589 g, 2.05 mmol) in CHCl₃ (21 mL) at 23° C. under N₂ was added N,N-diisopropylethylamine (0.794 g, 1.07 mL, 6.15 mmol) dropwise. The reaction was cooled to 0° C. and 4-bromo-2-fluoro-benzoyl chloride (0.511 g, 0.296 mL, 2.15 mmol) was added dropwise. The reaction was warmed to 23° C. for 18 hours. The mixture was concentrated. The residue was diluted with water (20 mL), sonicated to disperse the gummy solid, then stirred for 30 minutes with occasional sonication. The water was decanted from the gummy solid, and MeOH (20 mL) was added. After brief sonication and stirring for 15 minutes, the solid was collected by filtration and washed with a small amount of additional MeOH, then ether, then dried under vacuum to yield the title compound (0.7829 g, 85%) as a brown solid.

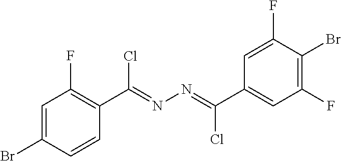 [SMILES: Fc1cc(Br)ccc1/C(Cl)=N/N=C(\Cl)c1cc(F)c(Br)c(F)c1]

Synthesis of (Z)-4-bromo-N—[(Z)-(4-bromo-2-fluorophenyl)(chloro)methylidene]-3,5-difluorobenzene-1-carbohydrazonoyl chloride: To a suspension of 4-bromo-3,5-difluoro-benzoic acid N′-(4-bromo-2-fluoro-benzoyl)-hydrazide (0.780 g, 1.73 mmol) in anhydrous toluene (8.6 mL) at 23° C. under. N₂ was gradually added PCl₅ (1.08 g, 5.18 mmol). The mixture was heated at 95° C. for 18 hours. The reaction was cooled to 23° C. and concentrated, suspended in toluene and concentrated again, then dried under vacuum to yield the title compound (0.7841 g, 93%) as yellow solid, which was used without purification.

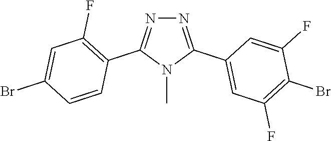 [SMILES: Cn1c(-c2cc(F)c(Br)c(F)c2)nnc1-c1ccc(Br)cc1F]

Synthesis of 3-(4-bromo-3,5-difluoro-phenyl)-5-(4-bromo-2-fluoro-phenyl)-4-methyl-4H-[1,2,4]triazole: To a solution of (Z)-4-bromo-N—[(Z)-(4-bromo-2-fluorophenyl)(chloro) methylidene]-3,5-difluorobenzene-1-carbohydrazonoyl chloride (0.7841 g, 1.60 mmol) in anhydrous toluene (8 mL) at 0° C. under N₂ was added methylamine solution (33% in EtOH; 2.00 mL, 16.0 mmol) dropwise. The reaction was warmed to 23° C. and was stirred for 18 hours. Acetic acid (2 mL) was added, and the mixture was heated to 60° C. for two hours before cooling to 23° C. and concentrating. The residue was partitioned between ethyl acetate (130 mL) and saturated NaHCO₃ solution (30 mL) and separated. The organic layer was washed with saturated NaHCO₃ solution (15 mL), water (15 mL), and brine (15 mL), then dried (Na₂SO₄), filtered, and concentrated. Silica gel chromatography (0 to 75% ethyl acetate; 100 to 25% CH₂Cl₂), followed by concentration and drying under vacuum, yielded the title compound (0.2883 g, 40%) as an off-white solid.

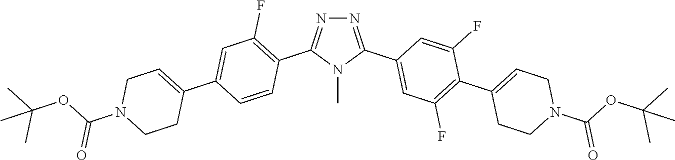 [SMILES: Cn1c(-c2cc(F)c(C3=CCN(C(=O)OC(C)(C)C)CC3)c(F)c2)nnc1-c1ccc(C2=CCN(C(=O)OC(C)(C)C)CC2)cc1F]

Synthesis of tert-butyl 4-{4-[5-(4-{1-[(tert-butoxy)carbonyl]-1,2,3,6-tetrahydro pyridin-4-yl}-2-fluorophenyl)-4-methyl-4H-1,2,4-triazol-3-yl]-2,6-difluorophenyl}-1,2,3,6-tetrahydropyridine-1-carboxylate: A solution of tert-butyl 5,6-dihydro-4-(4,4,5,5-tetramethyl-1,3,2-dioxaborolan-2-yl)pyridine-1(2H)-carboxylate (0.174 g, 0.564 mmol) in 1,4-dioxane (2.5 mL) i was degassed by bubbling nitrogen through the mixture for 5 minutes. After adding 2 M Na₂CO₃ solution (0.671 mL, 1.34 mmol), 3-(4-bromo-3,5-difluoro-phenyl)-5-(4-bromo-2-fluoro-phenyl)-4-methyl-4H-[1,2,4]triazole (0.120 g, 0.268 mmol), and 1,1′-bis(diphenylphosphino)ferrocene]dichloro palladium(II), CH₂Cl₂ adduct (0.0219 g, 0.0268 mmol), the reaction was degassed for two more minutes, then heated at 80° C. for 18 hours. The mixture was partitioned between CH₂Cl₂ (80 mL) and saturated NaHCO₃ solution (20 mL) and separated. The aqueous layer was re-extracted with CH₂Cl₂ (2×20 mL). The organic layers were combined and washed with brine (15 then dried (Na₂SO₄), filtered, and concentrated. Silica gel chromatography (0 to 80% ethyl acetate; 100 to 20% hexanes), followed by concentration and drying under vacuum, yielded the title compound (0.1682 g, 96%) as a tan foam.

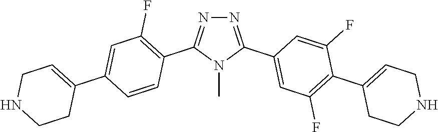 [SMILES: Cn1c(-c2cc(F)c(C3=CCNCC3)c(F)c2)nnc1-c1ccc(C2=CCNCC2)cc1F]

Synthesis of 4-(2,6-difluoro-4-{5-[2-fluoro-4-(1,2,3,6-tetrahydropyridin-4-yl)phenyl]-4-methyl-4H-1,2,4-triazol-3-yl}phenyl)-1,2,3,6-tetrahydropyridine hydrochloride: To a solution of tert-butyl 4-{4-[5-(4-{1-[(tert-butoxy)carbonyl]-1,2,3,6-tetrahydropyridin-4-yl}-2-fluoro phenyl)-4-methyl-4H-1,2,4-triazol-3-yl]-2,6-difluorophenyl}-1,2,3,6-tetrahydropyridine-1-carboxylate (0.1682 g, 0.258 mmol) and anisole (0.140 g, 0.141 mL, 1.29 mmol) in dichloromethane (3 mL) at 23° C. under N₂ was added 4 N HCl-1,4-dioxane solution (2.00 mL, 8.00 mmol) dropwise. After stirring for 18 hours, the mixture was concentrated, suspended in MeCN (2 mL) and concentrated again (2×), then dried under vacuum to yield the title compound (0.1447 g, quantitative) as an off-white solid.

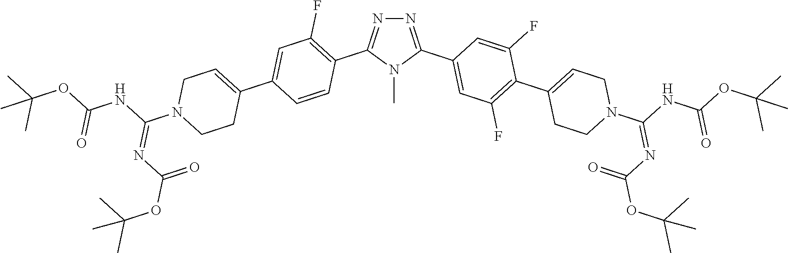 [SMILES: Cn1c(-c2cc(F)c(C3=CCN(/C(=N\C(=O)OC(C)(C)C)NC(=O)OC(C)(C)C)CC3)c(F)c2)nnc1-c1ccc(C2=CCN(/C(=N\C(=O)OC(C)(C)C)NC(=O)OC(C)(C)C)CC2)cc1F]

Synthesis of tert-butyl N—[(Z)-(4-{4-[5-(4-{1-[(Z)-{[(tert-butoxy)carbonyl]amino}({[(tert-butoxy)carbonyl]imino})methyl]-1,2,3,6-tetrahydropyridin-4-yl}-2-fluoro phenyl)-4-methyl-4H-1,2,4-triazol-3-yl]-2,6-difluorophenyl}-1,2,3,6-tetrahydropyridin-1-yl)({[(tert-butoxy)carbonyl]imino})methyl]carbamate: To a solution of 4-(2,6-difluoro-4-{5-[2-fluoro-4-(1,2,3,6-tetrahydro pyridin-4-yl)phenyl]-4-methyl-4H-1,2,4-triazol-3-yl}phenyl)-1,2,3,6-tetrahydropyridine hydrochloride (0.1447 g, 0.258 mmol) and N,N-diisopropylethylamine (0.233 g, 0.315 mL, 1.81 mmol) in N,N-dimethylformamide (4 mL) at 23° C. under N₂ was added tert-butyl N—[(Z)-{[(tert-butoxy)carbonyl]imino}(1H-pyrazol-1-yl)methyl]carbamate (0.240 g, 0.774 mmol). The suspension was stirred at 23° C. for 72 hours, then reaction was heated at 45° C. for 18 hours. The mixture was cooled to 23° C. and partitioned between ethyl acetate (130 mL) and saturated NH₄Cl solution (20 mL) and separated. The organic layer was washed again with saturated NH₄Cl solution (15 mL), saturated NaHCO₃ solution (15 mL), and brine (0.15 mL), then dried (Na₂SO₄), filtered, and concentrated. Silica gel chromatography (0 to 100% ethyl acetate; 100 to 0% CH₂Cl₂), followed by concentration and drying under vacuum, yielded the title compound (0.1579 g, 65%) as a light yellow foam.

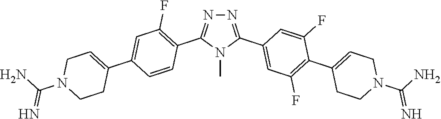 [SMILES: Cn1c(-c2cc(F)c(C3=CCN(C(=N)N)CC3)c(F)c2)nnc1-c1ccc(C2=CCN(C(=N)N)CC2)cc1F]

### Example 85

Synthesis of 4-(4-{5-[4-(1-carbamimidoyl-1,2,3,6-tetrahydropyridin-4-yl)-2-fluorophenyl]-4-methyl-4H-1,2,4-triazol-3-yl}-2,6-difluorophenyl)-1,2,3,6-tetrahydropyridine-1-carboximidamide trifluoroacetate: To a solution of tert-butyl N—[(Z)-(4-{4-[5-(4-{1-[(Z)-{[(tert-butoxy)carbonyl]amino}({[(tert-butoxy)carbonyl]imino})methyl]-1,2,3,6-tetrahydropyridin-4-yl}-2-fluorophenyl)-4-methyl-4H-1,2,4-triazol-3-yl]-2,6-difluorophenyl}-1,2,3,6-tetrahydropyridin-1-yl)({[(tert-butoxy)carbonyl]imino})methyl]carbamate (0.155 g, 0.166 mmol) and anisole (0.0895 g, 0.0902 mL, 0.828 mmol) in dichloromethane (3 mL) at 23° C. under N₂ was added 4 N HCl-1,4-dioxane solution (2.00 mL, 8.00 mmol) dropwise. After stirring for 18 hours, the mixture was concentrated, suspended in MeCN (2 mL) and concentrated again (2×), then dried under vacuum. The residue was purified by reverse-phase chromatography (SiliaSep C_{18 b 40} g column; 5 to 30% MeCN-water (both with 0.1% trifluoroacetic acid) over 22 min.). The fractions containing the title compound were combined and partially concentrated to remove most MeCN, then lyophilized to yield the title compound (0.0853 g, 67%) as a flocculent, white solid. ¹H NMR (300 MHz, DMSO-d₆) δ ppm 7.59-7.69 (m, 4H), 7.43-7.58 (m, 9H), 6.42-6.53 (m, 1H), 6.00-6.10 (m, 1H), 4.05-4.17 (m, 4H), 3.63 (m, J=−2.30 Hz, 7H), 2.60-2.68 (m, 2H), 2.50-2.55 (m, 2H). ¹⁹F NMR (282 MHz, DMSO-d₆) δ ppm −73.86 (s, 1F), 111.81-111.67 (m, 1F), −112.94-−112.79 (m, 1F). LC/MS (M+H)⁺=537.

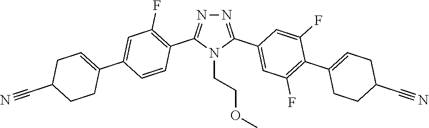 [SMILES: COCCn1c(-c2cc(F)c(C3=CCC(C#N)CC3)c(F)c2)nnc1-c1ccc(C2=CCC(C#N)CC2)cc1F]

Synthesis of 4-(4-{5-[4-(4-cyanocyclohex-1-en-1-yl)-2-fluorophenyl]-4-(2-methoxy ethyl)-4H-1,2,4-triazol-3-yl}-3-fluorophenyl)cyclohex-3-ene-1-carbonitrile: A suspension of 3,5-bis-(4-bromo-2-fluoro-phenyl)-4-(2-methoxy-ethyl)-4H-[1,2,4]triazole (0.600 g, 1.27 mmol) in 1,4-dioxane (6 mL) was degassed by bubbling nitrogen through the mixture for 5 minutes. After adding 2 M Na₂CO₃ solution (3.17 mL, 6.34 mmol), 4-(4,4,5,5-tetramethyl-[1,3,2]dioxaborolan-2-yl)-cyclohex-3-enecarbonitrile (0.621 g, 2.66 mmol), and 1,1′-bis(diphenylphosphino)ferrocene]dichloro palladium(II), CH₂Cl₂ adduct (0.104 g, 0.127 mmol), the reaction was degassed for two more minutes, heated at 80° C. for four hours, then cooled to 23° C. The mixture was partitioned between CH₂Cl₂ (80 mL) and saturated NaHCO₃ solution (20 mL) and separated. The aqueous layer was re-extracted with CH₂Cl₂ (2×20 mL). The organic layers were combined and washed with brine (15 mL), then dried (Na₂SO₄), filtered, and concentrated. Silica gel chromatography (0 to 100% (15% isopropanol-85% ethyl acetate); 100 to 0% hexanes), followed by concentration and drying under vacuum, yielded the title compound (0.6518 g, 98%) as a tan solid.

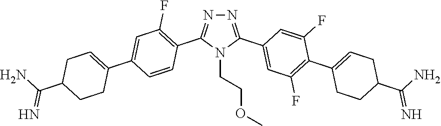 [SMILES: COCCn1c(-c2cc(F)c(C3=CCC(C(=N)N)CC3)c(F)c2)nnc1-c1ccc(C2=CCC(C(=N)N)CC2)cc1F]

### Example 86

Synthesis of 4-(4-{5-[4-(4-carbamimidoylcyclohex-1-en-1-yl)-2-fluoro phenyl]-4-(2-methoxyethyl)-4H-1,2,4-triazol-3-yl}-3-fluorophenyl)cyclohex-3-ene-1-carboximidamide trifluoroacetate: To a suspension of NH₄Cl (0.0407 g, 0.761 mmol) in anhydrous toluene (2 mL) at 0° C. under N₂ was added 2.0 M trimethylaluminum solution in toluene (0.381 mL, 0.761 mmol) dropwise. The ice bath was removed, and the mixture was stirred at 23° C. for two hours, during which time tiny bubbles of gas were slowly evolved. The reaction was again cooled to 0° C., before adding 4-(4-{5-[4-(4-cyanocyclohex-1-en-1-yl)-2-fluorophenyl]-4-(2-methoxyethyl)-4H-1,2,4-triazol-3-yl}-3-fluorophenyl)cyclohex-3-ene-1-carbonitrile (0.100 g, 0.190 mmol). The suspension was heated to 80° C. for 18 hours. In a separate vile, a solution of NH₄Cl (0.0814 g, 1.522 mmol) and 2.0 M trimethylaluminum solution in toluene (0.762 mL, 1.522 mmol) was prepared (in anhydrous toluene), then added to the reaction dropwise at 23° C. After heating to 80° C. for three hours, the suspension was stirred at 23° C. for 18 hours, and then the reaction was heated at 80° C. for 18 hours. The suspension was cooled to 23° C. and concentrated. The residue was purified by reverse-phase chromatography (SiliaSep C₁₈ 40 g column; 5 to 50% MeCN-water (both with 0.1% trifluoroacetic acid) over 23 minutes). The fractions containing the title compound were combined and partially concentrated to remove most MeCN, then lyophilized to yield the title compound (0.0853 g, 57%) as a flocculent, off-white solid. ¹H NMR (300 MHz, DMSO-d₆) δ ppm 8.86 (s, 4H) 8.76 (s, 4H) 7.57-7.66 (m, 2H) 7.45-7.56 (m, 4H) 6.37-6.49 (m, 2H) 4.01 (br t, J=4.40 Hz, 2H) 3.09 (t, J=5.27 Hz, 2H) 2.82 (s, 3H) 2.56-2.74 (m, 4H) 2.00-2.11 (m, 2H) 1.77-1.95 (m, 2H). ¹⁹F NMR (282 MHz, DMSO-d₆) δ ppm 74.06 (s, 1F) −75.24-−75.16 (m, 1F) −114.09-−113.96 (m, 1F). LC/MS (M+H)⁺=560.

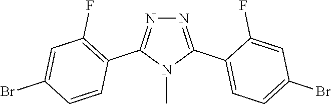 [SMILES: Cn1c(-c2ccc(Br)cc2F)nnc1-c1ccc(Br)cc1F]

Synthesis of 3,5-bis-(4-bromo-2-fluoro-phenyl)-4-methyl-4H-[1,2,4]triazole: To a solution of (Z)-4-bromo-N—[(Z)-(4-bromo-2-fluorophenyl)(chloro)methylidene]-2-fluorobenzene-1-carbohydrazonoyl chloride (1.06 g, 2.25 mmol) in anhydrous toluene (11 mL) 0° C. under N₂ was added methylamine solution (33% in EtOH, 2.80 mL, 22.5 mmol) dropwise. After 60 minutes, the reaction was permitted to warm to 23° C. and stirred for 18 hours. Acetic acid (3 mL) was added, and the mixture was heated to 60° C. for one hour before cooling to 23° C. and concentrating. The residue was partitioned between ethyl acetate (250 mL) and saturated NaHCO₃ solution (30 mL) and separated. The organic layer was washed with water (25 mL), and brine (25 mL), then dried (Na₂SO₄), filtered, and concentrated. Silica gel chromatography (0 to 100% (10% isopropanol-90% ethyl acetate); 100 to 0% CH₂Cl₂), followed by concentration and drying under vacuum, yielded the title compound (0.7979 g, 83%) as a flocculent, white solid.

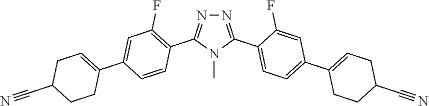 [SMILES: Cn1c(-c2ccc(C3=CCC(C#N)CC3)cc2F)nnc1-c1ccc(C2=CCC(C#N)CC2)cc1F]

Synthesis of 4-(4-{5-[4-(4-cyanocyclohex-1-en-1-yl)-2-fluorophenyl]-4-methyl-4H-1,2,4-triazol-3-yl}-3-fluorophenyl)cyclohex-3-ene-1-carbonitrile: A suspension of 3,5-bis-(4-bromo-2-fluoro-phenyl)-4-methyl-4H-[1,2,4]triazole (0.150 g, 0.350 mmol) in 1,4-dioxane (2.5 mL) was degassed by bubbling nitrogen through the mixture for 5 minutes. After adding 2 M Na₂CO₃ solution (0.874 mL, 1.75 mmol), 4-(4,4,5,5-tetramethyl-[1,3,2]dioxaborolan-2-yl)-cyclohex-3-enecarbonitrile (0.171 g, 0.734 mmol), and 1,1′-bis(diphenylphosphino)ferrocene]dichloro palladium(II), CH₂Cl₂ adduct (0.0285 g, 0.0350 mmol), the reaction was degassed for two more minutes, heated at 80° C. for 18 hours, then cooled to 23° C. The mixture was partitioned between CH₂Cl₂ (80 mL) and saturated NaHCO₃ solution (20 mL) and separated. The aqueous layer was re-extracted with CH₂Cl₂ (2×20 mL). The organic layers were combined and washed with brine (15 mL), then dried (Na₂SO₄), filtered, and concentrated. Silica gel chromatography (0 to 75% (10% isopropanol-90% ethyl acetate); 100 to 25% CH₂Cl₂), followed by concentration and drying under vacuum, yielded the title compound (0.1578 g, 94%) as a tan solid.

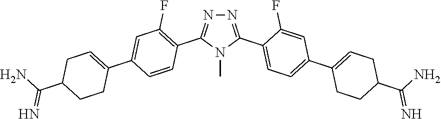 [SMILES: Cn1c(-c2ccc(C3=CCC(C(=N)N)CC3)cc2F)nnc1-c1ccc(C2=CCC(C(=N)N)CC2)cc1F]

### Example 87

Synthesis of 4-(4-{5-[4-(4-carbamimidoylcyclohex-1-en-1-yl)-2-fluoro phenyl]-4-methyl-4H-1,2,4-triazol-3-yl}-3-fluorophenyl)cyclohex-3-ene-1-carboximidamide trifluoroacetate: To a suspension of NH₄Cl (0.167 g, 3.12 mmol) in anhydrous toluene (3 mL) in a large scintillation vial at 0° C. under N₂ was added 2.0 M trimethylaluminum solution in toluene (1.56 mL, 3.12 mmol) dropwise. The ice bath was removed, and the mixture was stirred at 23° C. for two hours. The reaction was again cooled to 0° C., before adding 4-(4-{5-[4-(4-cyanocyclohex-1-en-1-yl)-2-fluorophenyl]-4-methyl-4H-1,2,4-triazol-3-yl}-3-fluorophenyl)cyclohex-3-ene-1-carbonitrile (0.150 g, 0.312 mmol) as a solid. The ice bath was removed, and the suspension was heated to 80° C. for 18 hours. The suspension was cooled to 23° C. and CHCl₃ (5 mL) and 6 N NaOH (2 mL) were added. After stirring for 30 minutes, the mixture was partitioned between 4:1 CHCl₃-isopropanol (80 mL) and saturated NaHCO₃ solution (35 mL) and separated. The aqueous layer was re-extracted with 4:1 CHCl₃-isopropanol (2×20 mL). The organic layers were combined and washed with brine (15 mL), then dried (Na₂SO₄), filtered, concentrated. The residue was purified by reverse-phase chromatography (SiliaSep C_{18 b 40} g column; 5 to 35% MeCN-water (both with 0.1% trifluoroacetic acid) over 23 minutes). The fractions containing the title compound were combined and partially concentrated to remove most of the MeCN, then lyophilized to yield the title compound (0.0823 g, 36%) as a flocculent, white solid. ¹H NMR. (300 MHz, DMSO-d₆) δ ppm 8.86 (s, 4H) 8.75 (s, 4H) 7.65 (t, 0.7=7.91 Hz, 2H) 7.45-7.58 (m, 4H) 6.35-6.50 (m, 2H) 3.43 (s, 3H) 2.51-2.75 (m, 6H) 1.99-2.12 (m, 2H) 1.76-1.97 (m, 2H). ¹⁹F NMR (282 MHz, DMSO-d₆) δ ppm −74.29 (s, 1F)-113.55-−113.44 (m, 1F). LC/MS (M+H)⁺=516.

### FORMULATIONS

The present invention also relates to compositions or formulations which comprise the antifungal agents according to the present disclosure. In general, the compositions of the present invention comprise an effective amount of one or more compounds of the disclosure and salts thereof according to the present invention which are effective for providing treatment of a fungal infection; and one or more excipients.

For the purposes of the present invention the term “excipient” and “carrier” are used interchangeably throughout the description of the present invention and said terms are defined herein as, “ingredients which are used in the practice of formulating a safe and effective pharmaceutical composition.”

The formulator will understand that excipients are used primarily to serve in delivering a safe, stable, and functional pharmaceutical, serving not only as part of the overall vehicle for delivery but also as a means for achieving effective absorption by the recipient of the active ingredient. An excipient may fill a role as simple and direct as being an inert filler, or an excipient as used herein may be part of a pH stabilizing system or coating to insure delivery of the ingredients safely to the stomach. The formulator can also take advantage of the fact the compounds of the present invention have improved cellular potency, pharmacokinetic properties, as well as improved oral bioavailability.

The present disclosure also provides pharmaceutical compositions that include at least one compound described herein and one or more pharmaceutically acceptable carriers, excipients, or diluents. Examples of such carriers are well known to those skilled in the art and can be prepared in accordance with acceptable pharmaceutical procedures, such as, for example, those described in Remington's Pharmaceutical Sciences, 17th edition, ed. Alfonso R. German), Mack Publishing Company, Easton, PA (1985), the entire disclosure of which is incorporated by reference herein for all purposes. As used herein, “pharmaceutically acceptable” refers to a substance that is acceptable for use in pharmaceutical applications from a toxicological perspective and does not adversely interact with the active ingredient. Accordingly; pharmaceutically acceptable carriers are those that are compatible with the other ingredients in the formulation and are biologically acceptable. Supplementary active ingredients can also be incorporated into the pharmaceutical compositions.

Compounds of the present disclosure can be administered orally or parenterally, neat or in combination with conventional pharmaceutical carriers. Applicable solid carriers can include one or more substances which can also act as flavoring agents, lubricants, solubilizers, suspending agents, fillers, glidants, compression aids, binders or tablet-disintegrating agents, or encapsulating materials. The compounds can be formulated in conventional manner, for example, in a manner similar to that used for known antifungal agents. Oral formulations containing a compound disclosed herein can comprise any conventionally used oral form, including tablets, capsules, buccal forms, troches, lozenges and oral liquids, suspensions or solutions. In powders, the carrier can be a finely divided solid, which is an admixture with a finely divided compound. In tablets, a compound disclosed herein can be mixed with a carrier having the necessary compression properties in suitable proportions and compacted in the shape and size desired. The powders and tablets can contain up to 99% of the compound.

Capsules can contain mixtures of one or more compound(s) disclosed herein with inert filler(s) and/or diluent(s) such as pharmaceutically acceptable starches (e.g., corn, potato or tapioca starch), sugars, artificial sweetening agents, powdered celluloses (e.g., crystalline and microcrystalline celluloses), flours, gelatins, gums, and the like.

Useful tablet formulations can be made by conventional compression, wet granulation or dry granulation methods and utilize pharmaceutically acceptable diluents, binding agents, lubricants, disintegrants, surface modifying agents (including surfactants), suspending or stabilizing agents, including, but not limited to, magnesium stearate, stearic acid, sodium lauryl sulfate, talc, sugars, lactose, dextrin, starch, gelatin, cellulose, methyl cellulose, microcrystalline cellulose, sodium carboxymethyl cellulose, carboxymethylcellulose calcium, polyvinylpyrrolidine, alginic acid, acacia gum, xanthan gum, sodium citrate, complex silicates, calcium carbonate, glycine, sucrose, sorbitol, dicalcium phosphate, calcium sulfate, lactose, kaolin, mannitol, sodium chloride, low melting waxes, and ion exchange resins. Surface modifying agents include nonionic and anionic surface modifying agents. Representative examples of surface modifying agents include, but are not limited to, poloxamer 188, benzalkonium chloride, calcium stearate, cetostearl alcohol, cetomacrogol emulsifying wax, sorbitan esters, colloidal silicon dioxide, phosphates, sodium dodecylsulfate, magnesium aluminum silicate, and triethanolamine. Oral formulations herein can utilize standard delay or time-release formulations to alter the absorption of the compound(s). The oral formulation can also consist of administering a compound disclosed herein in water or fruit juice, containing appropriate solubilizers or emulsifiers as needed.

Liquid carriers can be used in preparing solutions, suspensions, emulsions, syrups, elixirs, and for inhaled delivery. A compound of the present teachings can be dissolved or suspended in a pharmaceutically acceptable liquid carrier such as water, an organic solvent, or a mixture of both, or a pharmaceutically acceptable oils or fats. The liquid carrier can contain other suitable pharmaceutical additives such as solubilizers, emulsifiers, buffers, preservatives, sweeteners, flavoring agents, suspending agents, thickening agents, colors, viscosity regulators, stabilizers, and osmo-regulators. Examples of liquid carriers for oral and parenteral administration include, but are not limited to, water (particularly containing additives as described herein, e.g., cellulose derivatives such as a sodium carboxymethyl cellulose solution), alcohols (including monohydric alcohols and polyhydric alcohols, e.g., glycols) and their derivatives, and oils (e.g., fractionated coconut oil and arachis oil). For parenteral administration, the carrier can be an oily ester such as ethyl oleate and isopropyl myristate. Sterile liquid carriers are used in sterile liquid form compositions for parenteral administration. The liquid carrier for pressurized compositions can be halogenated hydrocarbon or other pharmaceutically acceptable propellants.

Liquid pharmaceutical compositions, which are sterile solutions or suspensions, can be utilized by, for example, intramuscular, intraperitoneal or subcutaneous injection. Sterile solutions can also be administered intravenously. Compositions for oral administration can be in either liquid or solid form.

Preferably the pharmaceutical composition is in unit dosage form, for example, as tablets, capsules, powders, solutions, suspensions, emulsions, granules, or suppositories. In such form, the pharmaceutical composition can be sub-divided in unit dose(s) containing appropriate quantities of the compound. The unit dosage forms can be packaged compositions, for example, packeted powders, vials, ampoules, prefilled syringes or sachets containing liquids. Alternatively, the unit dosage form can be a capsule or tablet itself, or it can be the appropriate number of any such compositions in package form. Such unit dosage form can contain from about 1 mg/kg of compound to about 500 mg/kg of compound, and can be given in a single dose or in two or more doses. Such doses can be administered in any manner useful in directing the compound(s) to the recipient's bloodstream, including orally, via implants, parenterally (including intravenous, intraperitoneal and subcutaneous injections), rectally, vaginally, and transdermally.

When administered for the treatment or inhibition of a particular disease state or disorder, it is understood that an effective dosage can vary depending upon the particular compound utilized, the mode of administration, and severity of the condition being treated, as well as the various physical factors related to the individual being treated. In therapeutic applications, a compound of the present teachings can be provided to a patient already suffering from a disease in an amount sufficient to cure or at least partially ameliorate the symptoms of the disease and its complications. The dosage to be used in the treatment of a specific individual typically must be subjectively determined by the attending physician. The variables involved include the specific condition and its state as well as the size, age and response pattern of the patient.

In some cases it may be desirable to administer a compound directly to the airways of the patient, using devices such as, but not limited to, metered dose inhalers, breath-operated inhalers, multidose dry-powder inhalers, pumps, squeeze-actuated nebulized spray dispensers, aerosol dispensers, and aerosol nebulizers. For administration by intranasal or intrabronchial inhalation, the compounds of the present teachings can be formulated into a liquid composition, a solid composition, or an aerosol composition. The liquid composition can include, by way of illustration, one or more compounds of the present teachings dissolved, partially dissolved, or suspended in one or more pharmaceutically acceptable solvents and can be administered by, for example, a pump or a squeeze-actuated nebulized spray dispenser. The solvents can be, for example, isotonic saline or bacteriostatic water. The solid composition can be, by way of illustration, a powder preparation including one or more compounds of the present teachings intermixed with lactose or other inert powders that are acceptable for intrabronchial use, and can be administered by, for example, an aerosol dispenser or a device that breaks or punctures a capsule encasing the solid composition and delivers the solid composition for inhalation. The aerosol composition can include, by way of illustration, one or more compounds of the present teachings, propellants, surfactants, and co-solvents, and can be administered by, for example, a metered device. The propellants can be a chlorofluorocarbon (CFC), a hydrofluoroalkane (HFA), or other propellants that are physiologically and environmentally acceptable.

Compounds described herein can be administered parenterally or intraperitoneally. Solutions or suspensions of these compounds or a pharmaceutically acceptable salts, hydrates, or esters thereof can be prepared in water suitably mixed with a surfactant such as hydroxyl-propylcellulose. Dispersions can also be prepared in glycerol, liquid polyethylene glycols, and mixtures thereof in oils. Under ordinary conditions of storage and use, these preparations typically contain a preservative to inhibit the growth of microorganisms.

The pharmaceutical forms suitable for injection can include sterile aqueous solutions or dispersions and sterile powders for the extemporaneous preparation of sterile injectable solutions or dispersions. In some embodiments, the form can sterile and its viscosity permits it to flow through a syringe. The form preferably is stable under the conditions of manufacture and storage and can be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), suitable mixtures thereof, and vegetable oils.

Compounds described herein can be administered transdermally, i.e., administered across the surface of the body and the inner linings of bodily passages including epithelial and mucosal tissues. Such administration can be carried out using the compounds of the present teachings including pharmaceutically acceptable salts, hydrates, or esters thereof, in lotions, creams, foams, patches, suspensions, solutions, and suppositories (rectal and vaginal).

Transdermal administration can be accomplished through the use of a transdermal patch containing a compound, such as a compound disclosed herein, and a carrier that can be inert to the compound, can be non-toxic to the skin, and can allow delivery of the compound for systemic absorption into the blood stream via the skin. The carrier can take any number of forms such as creams and ointments, pastes, gels, and occlusive devices. The creams and ointments can be viscous liquid or semisolid emulsions of either the oil-in-water or water-in-oil type. Pastes comprised of absorptive powders dispersed in petroleum or hydrophilic petroleum containing the compound can also be suitable. A variety of occlusive devices can be used to release the compound into the blood stream, such as a semi-permeable membrane covering a reservoir containing the compound with or without a carrier, or a matrix containing the compound. Other occlusive devices are known in the literature.

Compounds described herein can be administered rectally or vaginally in the form of a conventional suppository. Suppository formulations can be made from traditional materials, including cocoa butter, with or without the addition of waxes to alter the suppository's melting point, and glycerin. Water-soluble suppository bases, such as polyethylene glycols of various molecular weights, can also be used.

Lipid formulations or nanocapsules can be used to introduce compounds of the present disclosure into host cells either in vitro or in vivo. Lipid formulations and nanocapsules can be prepared by methods known in the art.

To increase the effectiveness of compounds of the present teachings, it can be desirable to combine a compound with other agents effective in the treatment of the target disease. For example, other active compounds (i.e., other active ingredients or agents) effective in treating the target disease can be administered with compounds of the present teachings. The other agents can be administered at the same time or at different times than the compounds disclosed herein.

Compounds of the present disclosure can be useful for the treatment or inhibition of a pathological condition or disorder in a mammal, for example, a human subject. The present teachings accordingly provide methods of treating or inhibiting a pathological condition or disorder by providing to a mammal a compound of the present teachings melding its pharmaceutically acceptable salt) or a pharmaceutical composition that includes one or more compounds of the present teachings in combination or association with pharmaceutically acceptable carriers. Compounds of the present teachings can be administered alone or in combination with other therapeutically effective compounds or therapies for the treatment or inhibition of the pathological condition or disorder.

Non-limiting examples of compositions according to the present invention include from about 0.00.1 mg to about 1000 mg of one or more compounds of the disclosure according to the present invention and one or more excipients; from about 0.01 mg to about 100 mg of one or more compounds of the disclosure according to the present invention and one or more excipients; and from about 0.1 mg to about 10 mg of one or more compounds of the disclosure according to the present invention; and one or more excipients.

### Procedures

The following procedures can be utilized in evaluating and selecting compounds as antifungal agents.

Fungal MIC Protocol: RPMI/MOPS media (Roswell Park Memorial Institute/3-morpholinopropane-1-sulfonic acid media) is prepared by dissolving 8.4 grams RPMI 0.1640 (Sigma cat #R1383), 34.52 grams MOPS buffer (Sigma cat #M3.183) and 2 grains glucose in 900 mL deionized water. The pH is adjusted to 7.0 with NaOH and filter sterilized. 0.1 mL, of a stock spore suspension (or loop from frozen stock) is inoculated to a 75 mL tissue culture flask (Falcon cat #353136) containing 50 mL of Potato Dextrose Agar (PDA, 24 grams PD broth (Fisher cat #DF 0549179), 20 grams agar (Fisher cat #BP 1423500) per liter water). Aspergillus and Fusarium strains are grown at 35° C. and Mucor strains are grown at room temperature for 3-5 days. Spores are harvested by flooding the flask with 5 mL phosphate buffered saline (PBS) pH 7.4+0.1% Tween-20 and several glass beads (Fisher cat #S800243) are added to aid in agitation. The supernatant is removed and the OD₅₃₀ of a 1:100 dilution is measured. Spores are diluted in RPMI/MOPS to a final OD₅₃₀ of 0.002 for Aspergillus and an OD₅₃₀ of 0.005 for Fusarium and Mucor. The final concentration of spores is approximately 2×10⁴ cfu/mL. Test compound(s) are diluted to 200 ug/mL in RPMI/MOPS (28 ul of 1.0 mg/mL DMSO stock in 1400 ul media). Ten (10) serial dilutions are prepared with RPMI/MOPS in a 96-well round bottom plate (Fisher cat #353136). For Mucor strains a sterile flat-bottom 96-well plate is used. 50 μl of compound dilutions in duplicate are added to a sterile 96-well round bottom plate. 50 ul of diluted spores are added to the compound-containing plates. Control wells include 1) compound but no cells and 2) spores but no compound. Mix the plates gently by hand, place in ziplock bags and

incubate at 35° C. for 48 hours. Score the MIC visually (lowest concentration of compound showing >50% growth inhibition) using an inverted mirror. For Mucorales strains the plates are read at OD530 and the concentration of compound that shows 50% growth inhibition is the MIC-50.

Candida Minimal Inhibitory Concentration (MIC): RPMI/MOPS media is prepared by dissolving 8.4 g RPMI 1640, 34.52 g MOPS buffer and 2 g glucose in 900 mL water. The pH is adjusted to 7.0 with NaOH. The total volume is brought to 1 L and the resulting solution is filter sterilized. The Candida stock is streaked on a Yeast extract Peptone Dextrose (YPD) agar plate and grown at 35° C. for isolation of single colonies. Compounds of the disclosure are diluted to 200 μg/mL in RPMI/MOPS (4.8 ul of 10 mg/mL DMSO stock in 240 ul media). Ten (10) 1:2 serial dilutions are prepared in RPMI/MOPS in a 96-well round bottom plate. Fifty (50) μl of compound dilutions are transferred in duplicate to a sterile tissue culture-treated 96-well flat-bottom, black-sided polypropylene plate. A single colony of Candida from the YPD plate is re-suspended in 5 mL phosphate buffered saline (BS) and optical density (OD) at 600 nm is measured. The suspension is adjusted to OD=1.0 and a 1:1000 dilution is prepared in RPMI/MOPS. Fifty (50) ul aliquots of the diluted yeast are added to the plates containing 50 μL of compound to all wells except 12E-H. Control wells include: 12 A-D cells, no compound and 12 E-H no cells, no compound. The plates are mixed gently by hand and placed in Ziplok bags in a 35° C. incubator. The OD600 is measured at 24 and 48 hours. The lowest concentration of a compound that shows a significant reduction in fungal growth is recorded as the MIC and MIC-50 is the concentration of compound that shows 50% growth inhibition

TABLE 37
Fungal strains employed in Fungal MIC Protocol

| ATCC Fungal Strains | Vendor | Catalog # |
|---|---|---|
| Aspergillus flavus AFL1 | ATCC | MYA-3631 |
| Aspergillus fumigatus T33439 | ATCC | MYA-3626 |
| Fusarium falciforme FS1 | ATCC | MYA-3636 |
| Fusarium solani | ATCC | 58877 |
| Mucor circinelloides 1463(62) | ATCC | 26759 |
| Mucor ramosissimus 057718 | ATCC | 90286 |

TABLE 38
Antifungal activity of exemplary compounds of the disclosure.

| | MIC (mcg · mL) | | | | MIC-50 (mcg/mL) | | |
|---|---|---|---|---|---|---|---|
| | Aspergillus | Aspergillus | Fusarium | Fusarium | Mucorales | Mucorales | Candida |
| | flavus | fumigatus | falciforme | solani | circinelloides | ramosissimus | albicans |
| Example | 3631 | 3626 | 3636 | 58877 | 26579 | 90286 | GDH2346 |
| 1 | 0.1 | <0.05 | <0.05 | 0.78 | 0.2 | 0.78 | <0.2 |
| 2 | <0.1 | 0.2 | <0.1 | <0.1 | >100 | >100 | <0.024 |
| 3 | <0.1 | <0.1 | <0.1 | <0.1 | 0.39 | 0.78 | 0.1 |
| 4 | 0.39 | <0.1 | <0.1 | 0.2 | 0.39 | 1.56 | |
| 5 | 1.56 | 0.39 | <0.1 | 0.39 | 1.56 | 6.25 | |
| 6 | 0.39 | 0.2 | <0.1 | 0.39 | 0.78 | 1.56 | 3.13 |
| 7 | 0.78 | <0.1 | <0.1 | 0.39 | 0.78 | 1.56 | 3.13 |
| 8 | 0.05 | 0.05 | 0.006 | 0.05 | 0.2 | 0.39 | 0.1 |
| 9 | 0.39 | 0.2 | 0.2 | 0.2 | 3.13 | 3.13 | <0.1 |
| 10 | <0.1 | <0.1 | <0.1 | <0.1 | 0.78 | 1.56 | <0.1 |
| 11 | 0.78 | 0.39 | 0.2 | 0.2 | 1.56 | 3.13 | 1.56 |
| 12 | 3.13 | 3.13 | 0.39 | <0.1 | >100 | >100 | 12.5 |
| 13 | 0.2 | <0.1 | 0.78 | 6.25 | 0.39 | 1.56 | 0.2 |
| 14 | <0.1 | <0.1 | <0.1 | 6.25 | <0.1 | 1.56 | 0.39 |
| 15 | 3.13 | 3.13 | 0.78 | <0.1 | >100 | >100 | 25 |
| 16 | 0.39 | 0.39 | <0.1 | <0.1 | 12.5 | >100 | 0.39 |
| 17 | 0.39 | <0.1 | <0.1 | 0.39 | 0.39 | 0.78 | 3.13 |
| 18 | 0.78 | 0.39 | <0.1 | 0.39 | 0.78 | 0.78 | |
| 19 | 0.03 | 0.01 | 0.01 | 0.03 | 0.1 | 0.2 | 0.1 |
| 20 | 0.2 | <0.1 | <0.1 | 0.2 | 0.78 | 12.5 | 0.78 |
| 21 | 0.39 | 0.2 | 0.2 | 0.2 | 3.13 | 6.25 | 0.3 |
| 22 | 0.39 | 0.78 | 0.78 | 0.2 | 1.56 | 6.25 | 0.16 |
| 23 | 0.78 | 0.39 | 0.39 | 0.2 | 0.39 | 1.56 | 0.39 |
| 24 | 1.56 | 0.78 | 0.78 | 0.78 | 6.25 | 50 | 1.56 |
| 25 | 0.39 | 0.2 | 0.2 | 0.2 | 0.78 | 3.13 | 0.78 |
| 26 | 12.5 | 100 | | | 12.5 | 100 | |
| 27 | 0.39 | <0.1 | <0.1 | 0.2 | 0.78 | 6.25 | 0.39 |
| 28 | <0.1 | <0.1 | <0.1 | <0.1 | >100 | >100 | |
| 29 | 0.39 | <0.1 | <0.1 | 0.39 | 6.25 | 6.25 | 1.56 |
| 30 | 0.06 | 0.01 | 0.01 | <0.05 | 0.1 | 0.78 | 0.63 |
| 31 | <0.05 | <0.05 | <0.05 | <0.05 | 0.78 | 1.56 | 0.1 |
| 32 | 0.39 | 0.78 | <0.1 | 0.78 | 0.78 | 6.25 | 0.08 |
| 33 | 0.04 | 0.63 | 0.05 | 0.025 | 3.13 | 12.5 | 0.08 |
| 34 | 0.2 | 0.2 | <0.1 | 0.39 | 0.39 | 25 | 0.16 |
| 35 | 0.39 | 1.56 | <0.1 | 1.56 | 0.39 | 25 | 0.39 |
| 36 | 0.2 | 0.2 | 0.1 | 0.2 | 0.78 | 3.13 | 0.39 |
| 37 | 0.2 | 0.39 | 0.39 | 0.39 | 0.39 | 6.25 | 0.78 |
| 38 | 0.16 | 0.2 | 0.2 | <0.1 | 3.13 | 0.78 | 6.25 |
| 39 | 1.56 | 1.56 | 0.78 | 0.78 | >100 | >100 | 0.78 |
| 40 | 6.25 | 6.25 | 1.56 | 1.56 | 6.25 | 50 | |
| 41 | 3.13 | 3.13 | 0.39 | 6.25 | 100 | 100 | 0.39 |
| 42 | 6.25 | >25 | 0.39 | 1.56 | 25 | 25 | 0.39 |
| 43 | 0.31 | 0.78 | 0.2 | 0.78 | 12.5 | 25 | 0.39 |
| 44 | 1.56 | 6.25 | | | | | 0.39 |
| 45 | 0.39 | 0.39 | | | | | 0.39 |
| 46 | 1.56 | 1.56 | | | | | 0.2 |
| 47a | 0.39 | 1.56 | | | | | 0.78 |
| 47b | 0.78 | 0.78 | | | | | 1.56 |
| 48 | 0.78 | 0.78 | <0.1 | 3.13 | 0.78 | 6.25 | 1.56 |
| 49 | 0.78 | 3.13 | <0.1 | 3.13 | 1.56 | 25 | 1.56 |
| 50 | 0.39 | 0.78 | <0.1 | 1.56 | 50 | 25 | 0.78 |
| 51 | 6.25 | 0.78 | | | | | 3.13 |
| 52 | 6.25 | 50 | | | | | 4.3 |
| 53 | 6.25 | 25 | | | | | 1.56 |
| 54 | 0.39 | 1.56 | <0.1 | 0.78 | 0.39 | 3.13 | 0.5 |
| 55 | 3.13 | 0.78 | | | | | 1.56 |
| 56 | 0.39 | 0.39 | <0.1 | <0.1 | 0.39 | 1.56 | 0.78 |
| 57 | 0.2 | <0.1 | <0.1 | <0.1 | 0.39 | 3.13 | 0.2 |
| 60 | 25 | 25 | | | | | 1.56 |
| 61 | 3.13 | 0.78 | | | | | 1.56 |
| 66 | 0.39 | 0.2 | 0.1 | 0.2 | 1.56 | 6.25 | |
| 67 | 0.1 | 0.1 | 0.05 | 0.05 | 0.39 | 1.56 | 0.2 |
| 68 | <0.1 | 0.1 | <0.1 | <0.1 | 0.39 | 0.78 | 0.2 |
| 69 | 0.2 | 0.1 | <0.1 | <0.1 | 0.39 | 0.39 | 0.39 |
| 70 | 0.1 | 0.1 | 0.05 | 0.05 | 1.56 | 12.5 | 0.3 |
| 71 | 3.13 | 1.56 | 1.56 | 0.39 | 25 | 25 | |
| 72 | 0.78 | 0.39 | 0.2 | 0.1 | 6.25 | 12.5 | |
| 73 | 0.2 | 0.1 | 0.1 | 0.05 | 1.56 | 3.13 | 0.2 |
| 74 | 0.78 | 0.39 | 0.1 | 0.1 | 1.56 | 3.13 | |
| 75 | 3.13 | 1.56 | 1.56 | 0.39 | 25 | 25 | |
| 76 | 3.13 | 0.78 | 0.78 | 0.39 | 25 | 25 | |
| 77 | 0.78 | 0.2 | 0.2 | 0.2 | 6.25 | 25 | |
| 78 | 0.1 | 0.05 | 0.05 | 0.1 | 0.2 | 0.78 | 0.2 |
| 79 | 0.1 | 0.1 | 0.05 | 0.1 | 0.2 | 0.39 | 0.1 |
| 80 | 25 | 25 | 25 | 25 | 25 | 25 | |
| 81 | 0.78 | 0.39 | 0.1 | 0.1 | 1.56 | 6.25 | |
| 82 | 0.2 | 0.1 | 0.05 | 0.05 | 0.39 | 1.56 | 0.2 |
| 83 | 0.78 | 0.39 | 0.2 | 0.2 | 1.56 | 12.5 | |
| 84 | 0.39 | 0.2 | 0.1 | 0.05 | 0.39 | 0.78 | 0.2 |
| 85 | 0.024 | 0.024 | <0.012 | <0.012 | 0.2 | 0.78 | 0.1 |
| 86 | 0.78 | 0.78 | 0.78 | 0.2 | 1.56 | 6.25 | 0.78 |
| 87 | 0.78 | 0.2 | 0.1 | 0.2 | 0.39 | 3.13 | 0.2 |

MIC: Minimal Inhibitory Concentration, 100% growth inhibition at 48 hours. MIC-50: Minimal Inhibitory Concentration, 50% growth inhibition at 48 hours. Mcg: microgram

1-43. (canceled)

44. A compound having formula (I): 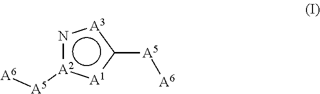
including enantiomers, diastereomers, hydrates, solvates, pharmaceutically acceptable salts, prodrugs and complexes thereof, wherein:
A¹ is selected from the group consisting of CR¹, O, N, and NR¹;
when A¹ is CR¹, A², A³ are N;
alternately, when A¹ is CR¹, A² is C, and A³ is NR^{1a};
when A¹ is O, A² is C, and A³ is N;
when A¹ is NR¹, A² is C, and A³ is N;
when A¹ is N, A² is C, and A³ is NR^{1a};
A⁵ is at each occurrence independently selected from the group consisting of 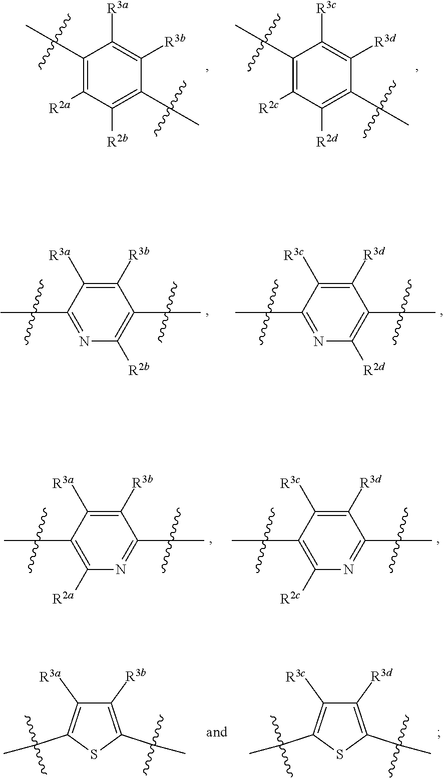
A⁶ is at each occurrence independently selected from the group consisting of, 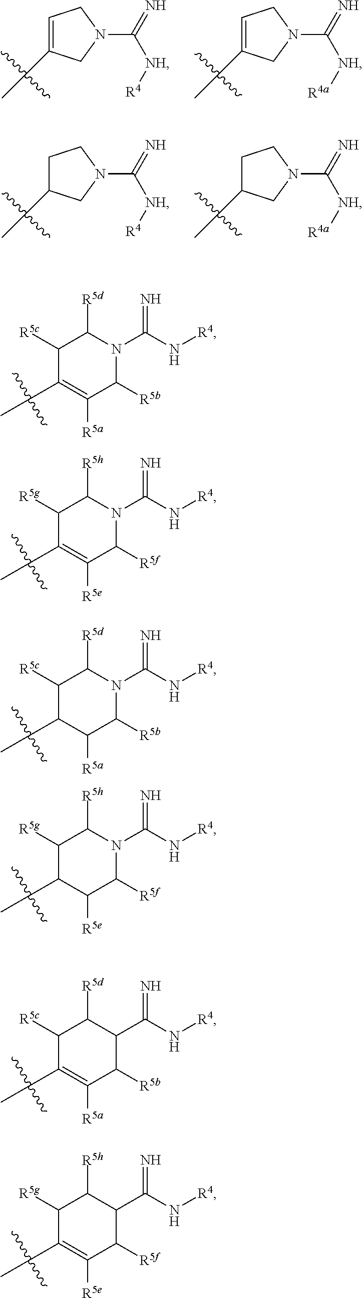 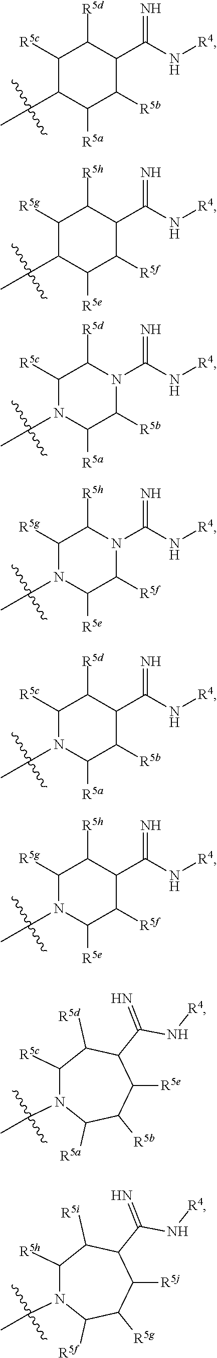 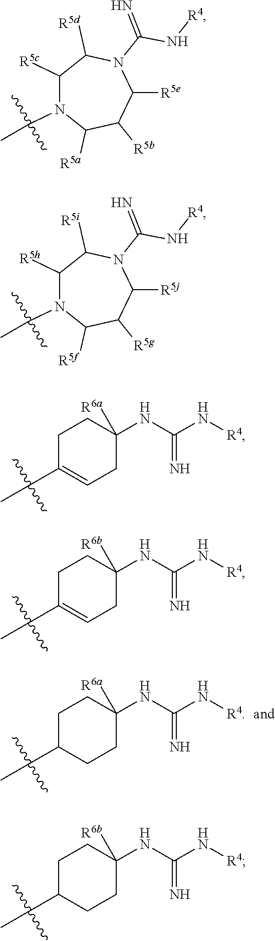
R¹ is selected from the group consisting of hydrogen C₁₋₈ alkyl, C₃₋₈ branched alkyl, C₃₋₈ cycloalkyl, optionally substituted benzyl, 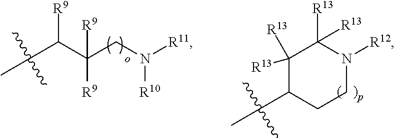
R^{1a} is selected from the group consisting of hydrogen, C₁₋₈ alkyl, C₃₋₈ branched alkyl, C₃₋₈ cycloalkyl, optionally substituted benzyl, 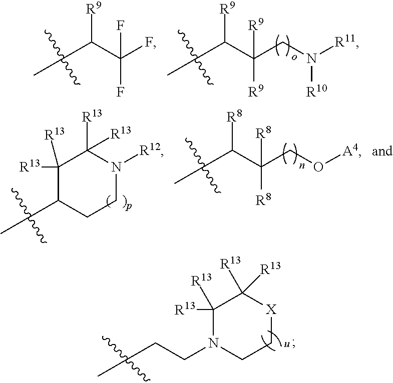
A⁴ is selected from the group consisting of hydrogen, C₁₋₄ alkyl, C₃₋₅ branched alkyl, 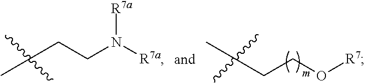
R⁷ is selected from the group consisting of hydrogen, C₁₋₄ alkyl and C₃₋₅ branched alkyl;
R^{7a} is selected from the group consisting of C₁₋₄ alkyl, C₃₋₈ branched alkyl, and C₃₋₈ cycloalkyl;
R⁸ is at each occurrence independently selected from the group consisting of hydrogen, and C₁₋₄ alkyl;
R^{8a} is selected from the group consisting of hydrogen, C₁₋₄ alkyl, and 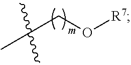
R⁹ is at each occurrence independently selected from the group consisting of hydrogen and C₁₋₄ alkyl;
R¹⁰ is selected from the group consisting of hydrogen, C₁₋₄ alkyl, C₃₋₈ branched alkyl, and C₃₋₈ cycloalkyl;
R¹¹ is selected from the group consisting of C₁₋₄ alkyl, C₃₋₈ branched alkyl, C₃₋₈ cycloalkyl, and 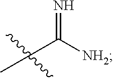 [SMILES: *C(=N)N]
in some embodiments R¹⁰ and R¹¹ are optionally joined to form a heterocyclic ring consisting of three, four, five, six, or seven members;
R¹² is selected from the group consisting of C₁₋₄ alkyl, C₃₋₈ branched alkyl, C₃₋₈ cycloalkyl, and 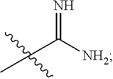 [SMILES: *C(=N)N]
R¹³ is at each occurrence independently selected from the group consisting of hydrogen, and C₁₋₄ alkyl;
p is 0, 1, or 2;
o is 0, 1, or 2;
n is 0, 1, or 2;
m is 1, 2, or 3;
u is 1 or 2;
X is selected from the group consisting of NR¹², oxygen, sulfur, and SO₂;
R^{2a} is selected from the group consisting of hydrogen, C₁₋₄ alkyl, fluorine, chlorine, C₁₋₄-alkoxy, CN, OCF₃, and CF₃;
R^{2b} is selected from the group consisting of hydrogen, C₁₋₄ alkyl, fluorine, chlorine, C₁₋₄-alkoxy, CN, OCF₃, and CF₃;
R^{2c} is selected from the group consisting of hydrogen, C₁₋₄ alkyl, fluorine, chlorine, C₁₋₄-alkoxy, CN, OCF₃, and CF₃;
R^{2d} is selected from the group consisting of hydrogen, C₁₋₄ alkyl, fluorine, chlorine, C₁₋₄-alkoxy, CN, OCF₃, and CF₃;
R^{3a} is selected from the group consisting of hydrogen, C₁₋₄ alkyl, fluorine, chlorine, C₁₋₄-alkoxy, CN, OCF₃, and CF₃;
R^{3b} is selected from the group consisting of hydrogen, C₁₋₄ alkyl, fluorine,
chlorine, C₁₋₄-alkoxy, CN, OCF₃, and CF₃;
R^{3c} is selected from the group consisting of hydrogen, C₁₋₄ alkyl, fluorine, chlorine, C₁₋₄-alkoxy, CN, OCF₃, and CF₃;
R^{3d} is selected from the group consisting of hydrogen, C₁₋₄ alkyl, fluorine, chlorine, C₁₋₄-alkoxy, CN, OCF₃, and CF₃;
R⁴ is selected from the group consisting of hydrogen and C₁₋₄ alkyl;
R^{4a} is selected from the group consisting of hydrogen and C₁₋₄ alkyl;
R^{5a} is selected from the group consisting of hydrogen and C₁₋₄ alkyl;
R^{5b} is selected from the group consisting of hydrogen and C₁₋₄ alkyl;
R^{5c} is selected from the group consisting of hydrogen and C₁₋₄ alkyl;
R^{5d} is selected from the group consisting of hydrogen and C₁₋₄ alkyl;
R^{5e} is selected from the group consisting of hydrogen and C₁₋₄ alkyl;
R^{5f} is selected from the group consisting of hydrogen and C₁₋₄ alkyl;
R^{5g} is selected from the group consisting of hydrogen and C₁₋₄ alkyl;
R^{5h} is selected from the group consisting of hydrogen and C₁₋₄ alkyl;
R⁵ⁱ is selected from the group consisting of hydrogen and C₁₋₄ alkyl;
R^{5j} is selected from the group consisting of hydrogen and C₁₋₄ alkyl;
R^{6a} is selected from the group consisting of hydrogen and C₁₋₄ alkyl;
R^{6b} is selected from the group consisting of hydrogen and C₁₋₄ alkyl.

45. The compound of claim 44 having the formula (II) 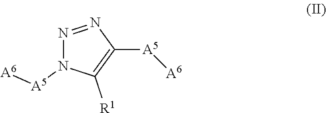
including enantiomers, diastereomers, hydrates, solvates, pharmaceutically acceptable salts, and complexes thereof.

46. The compound of claim 44 having the formula (III) 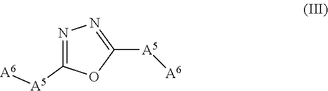
including enantiomers, diastereomers, hydrates, solvates, pharmaceutically acceptable salts, and complexes thereof.

47. The compound of claim 44 having the formula (IV) 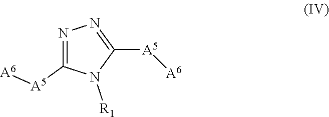
including enantiomers, diastereomers, hydrates, solvates, pharmaceutically acceptable salts, and complexes thereof.

48. The compound of claim 44 having the formula (V) 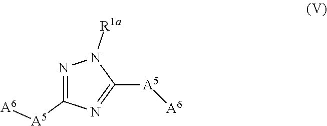
including enantiomers, diastereomers, hydrates, solvates, pharmaceutically acceptable salts, and complexes thereof.

49. The compound of claim 44 having the formula (VI) 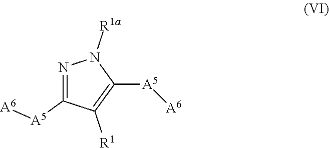
including enantiomers, diastereomers, hydrates, solvates, pharmaceutically acceptable salts, and complexes thereof.

50. The compound of claim 44 having the formula (VII) 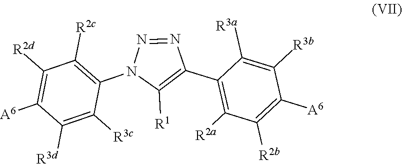
including enantiomers, diastereomers, hydrates, solvates, pharmaceutically acceptable salts, and complexes thereof.

51. The compound of claim 44 having the formula (VIII) 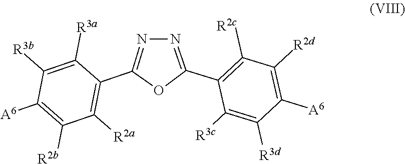
including enantiomers, diastereomers, hydrates, solvates, pharmaceutically acceptable salts, and complexes thereof.

52. The compound of claim 44 having the formula (IX) 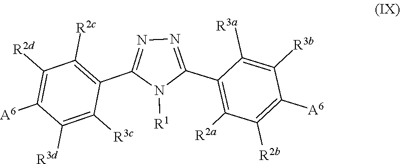
including enantiomers, diastereomers, hydrates, solvates, pharmaceutically acceptable salts, and complexes thereof.

53. The compound of claim 44 having the formula (X) 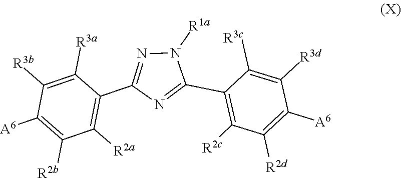
including enantiomers, diastereomers, hydrates, solvates, pharmaceutically acceptable salts, and complexes thereof.

54. The compound of claim 44 having the formula (XI) 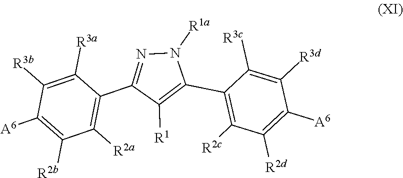
including enantiomers, diastereomers, hydrates, solvates, pharmaceutically acceptable salts, and complexes thereof.

55. The compound according to claim 44 that is
4,4′-(4,4′-(4-Methyl-4H-1,2,4-triazole-3,5-diyl)bis(4,1-phenylene))bis(5,6-dihydropyridine-1(2H)-carboximidamide);
4,4′-(4,4′-(4-Ethyl-4H-1,2,4-triazole-3,5-diyl)bis(4,1-phenylene)) bis(5,6-dihydropyridine-1(2H)-carboximidamide);
4,4′-(4,4′-(4-Isopropyl-4H-1,2,4-triazole-3,5-diyl)bis(4,1-phenylene))bis(5,6-dihydropyridine-1(2H)-carboximidamide);
4,4′-(4,4′-(4-Cyclopropyl-4H-1,2,4-triazole-3,5-diyl)bis(4,1-phenylene)) bis(5,6-dihydropyridine-1(2H)-carboximidamide);
4,4′-(4,4′-(4-Benzyl-4H-1,2,4-triazole-3,5-diyl)bis(4,1-phenylene))bis(5,6-dihydropyridine-1(2H)-carboximidamide);
4-(4-(4-Benzyl-5-(4-(1-carbamimidoyl-1,2,3,6-tetrahydropyridin-4-yl)-2-fluorophenyl)-4H-1,2,4-triazol-3-yl)phenyl)-5,6-dihydropyridine-1(2H)-carboximidamide;
4,4′-(4,4′-(4-Methyl-4H-1,2,4-triazole-3,5-diyl)bis(3-fluoro-4,1-phenylene))bis(5,6-dihydropyridine-1(2H)-carboximidamide);
4,4′-(4,4′-(4-(2-(2-Ethoxyethoxy)ethyl)-4H-1,2,4-triazole-3,5-diyl)bis(3-fluoro-4,1-phenylene))bis(5,6-dihydropyridine-1(2H)-carboximidamide);
4,4′-(4,4′-(4-Ethyl-4H-1,2,4-triazole-3,5-diyl)bis(3-fluoro-4,1-phenylene)) bis(5,6-dihydropyridine-1(2H)-carboximidamide)′
4,4′-(4,4′-(4-Octyl-4H-1,2,4-triazole-3,5-diyl)bis(3-fluoro-4,1-phenylene))bis(5,6-dihydropyridine-1(2H)-carboximidamide);
4,4′-(4,4′-(4-(1-Carbamimidoylpiperidin-4-yl)-4H-1,2,4-triazole-3,5-diyl)bis (3-fluoro-4,1-phenylene))bis(5,6-dihydropyridine-1(2H)-carboximidamide);
4,4′-(4,4′-(4-Isopropyl-4H-1,2,4-triazole-3,5-diyl)bis(3-fluoro-4,1-phenylene))bis(5,6-dihydropyridine-1(2H)-carboximidamide);
4,4′-(4,4′-(4-Isobutyl-4H-1,2,4-triazole-3,5-diyl)bis(3-fluoro-4,1-phenylene)) bis(5,6-dihydropyridine-1(2H)-carboximidamide));
4,4′-(4,4′-(4-(1-Methylpiperidin-4-yl)-4H-1,2,4-triazole-3,5-diyl)bis(3-fluoro-4,1-phenylene))bis(5,6-dihydropyridine-1(2H)-carboximidamide);
4,4′-(4,4′-(4-(2-guanidinoethyl)-4H-1,2,4-triazole-3,5-diyl)bis(3-fluoro-4,1-phenylene))bis(5,6-dihydropyridine-1(2H)-carboximidamide);
4,4′-(4,4′-(4-Cyclopropyl-4H-1,2,4-triazole-3,5-diyl)bis(3-fluoro-4,1-phenylene))bis(5,6-dihydropyridine-1(2H)-carboximidamide);
4,4′-(4,4′-(4-Benzyl-4H-1,2,4-triazole-3,5-diyl)bis(3-fluoro-4,1-phenylene)) bis(5,6-dihydropyridine-1(2H)-carboximidamide;
4,4′-(4,4′-(4-Methyl-4H-1,2,4-triazole-3,5-diyl)bis(2-fluoro-4,1-phenylene)) bis(5,6-dihydropyridine-1(2H)-carboximidamide);
4,4′-(4,4′-(4-Methyl-4H-1,2,4-triazole-3,5-diyl)bis(2,5-difluoro-4,1-phenylene))bis(5,6-dihydropyridine-1(2H)-carboximidamide);
4-(4-(5-(4-(1-Carbamimidoyl-1,2,3,6-tetrahydropyridin-4-yl)-2-methylphenyl)-4-methyl-4H-1,2,4-triazol-3-yl)phenyl)-5,6-dihydropyridine-1(2H)-carboximidamide;
4,4′-(4,4′-(4-Methyl-4H-1,2,4-triazole-3,5-diyl)bis(3-methyl-4,1-phenylene))bis(5,6-dihydropyridine-1(2H)-carboximidamide);
4-(4-(5-(4-(1-Carbamimidoyl-1,2,3,6-tetrahydropyridin-4-yl)-2-fluoro-6-(methylamino)phenyl)-4-methyl-4H-1,2,4-triazol-3-yl)phenyl)-5,6-dihydropyridine-1(2H)-carboximidamide;
4,4′-(4,4′-(4-Methyl-4H-1,2,4-triazole-3,5-diyl)bis(2-methyl-4,1-phenylene)) bis(5,6-dihydropyridine-1(2H)-carboximidamide);
4-(4-(5-(4-(1-Carbamimidoyl-1,2,3,6-tetrahydropyridin-4-yl)-3-(trifluoromethyl)phenyl)-4-methyl-4H-1,2,4-triazol-3-yl)phenyl)-5,6-dihydropyridine-1(2H)-carboximidamide;
4-(4-(5-(4-(1-Carbamimidoyl-1,2,3,6-tetrahydropyridin-4-yl)-2-(isopropylamino)phenyl)-4-isopropyl-4H-1,2,4-triazol-3-yl)-3-fluorophenyl)-5,6-dihydro pyridine-1(2H)-carboximidamide;
3-(4-(5-(4-(1-Carbamimidoyl-2,5-dihydro-1H-pyrrol-3-yl)-3-(trifluoromethyl)phenyl)-4-methyl-4H-1,2,4-triazol-3-yl)phenyl)-2,5-dihydro-1H-pyrrole-1-carboximidamide;
3,3′-(4,4′-(4-Methyl-4H-1,2,4-triazole-3,5-diyl)bis(3-fluoro-4,1-phenylene)) bis(2,5-dihydro-1H-pyrrole-1-carboximidamide);
3,3′-(4,4′-(4-Methyl-4H-1,2,4-triazole-3,5-diyl)bis(4,1-phenylene))bis(2,5-dihydro-1H-pyrrole-1-carboximidamide;
4-(4-{1-[4-(1-Carbamimidoyl-1,2,3,6-tetrahydropyridin-4-yl) phenyl]-1H-1,2,3-triazol-4-yl}phenyl)-1,2,3,6-tetrahydropyridine-1-carboximidamide;
4-(4-{1-[4-(1-Carbamimidoyl-1,2,3,6-tetrahydropyridin-4-yl)phenyl]-5-methyl-1H-1,2,3-triazol-4-yl}phenyl)-1,2,3,6-tetrahydropyridine-1-carboximidamide;
4-(4-(1-(4-(1-Carbamimidoyl-1,2,3,6-tetrahydropyridin-4-yl)-2-methyl phenyl)-5-methyl-1H-1,2,3-triazol-4-yl)phenyl)-5,6-dihydropyridine-1(2H)-carboximidamide;
4-(4-(1-(4-(1-Carbamimidoyl-1,2,3,6-tetrahydropyridin-4-yl)-3-methylphenyl)-5-methyl-1H-1,2,3-triazol-4-yl)phenyl)-5,6-dihydropyridine-1(2H)-carboximidamide;
4-(4-(1-(4-(1-Carbamimidoyl-1,2,3,6-tetrahydropyridin-4-yl)-2-methylphenyl)-1H-1,2,3-triazol-4-yl)phenyl)-5,6-dihydropyridine-1(2H)-carboximidamide;
4-(4-(1-(4-(1-Carbamimidoyl-1,2,3,6-tetrahydropyridin-4-yl)-3-methyl phenyl)-1H-1,2,3-triazol-4-yl)phenyl)-5,6-dihydropyridine-1(2H)-carboximidamide;
4-(4-(4-(4-(1-Carbamimidoyl-1,2,3,6-tetrahydropyridin-4-yl)-2-methylphenyl)-1H-1,2,3-triazol-1-yl)phenyl)-5,6-dihydropyridine-1(2H)-carboximidamide;
4-(4-(4-(4-(1-Carbamimidoyl-1,2,3,6-tetrahydropyridin-4-yl)-3-methyl phenyl)-1H-1,2,3-triazol-1-yl)phenyl)-5,6-dihydropyridine-1(2H)-carboximidamide;
4,4′-(4,4′-(1-Methyl-1H-1,2,4-triazole-3,5-diyl)bis(4,1-phenylene)) bis(5,6-dihydropyridine-1(2H)-carboximidamide);
4,4′-(4,4′-(4-Methyl-4H-1,2,4-triazole-3,5-diyl)bis(4,1-phenylene))dipiperidine-1-carboximidamide;
1-[4-(4-{5-[4-(4-Carbamimidamidocyclohex-1-en-1-yl)-2-fluorophenyl]-4-methyl-4H-1,2,4-triazol-3-yl}-3-fluorophenyl)cyclohex-3-en-1-yl]guanidine;
4-(4-{5-[4-(1-Carbamimidoyl-1,2,3,6-tetrahydropyridin-4-yl)phenyl]-1,3,4-oxadiazol-2-yl}phenyl)-1,2,3,6-tetrahydropyridine-1-carboximidamide;
4-(4-(5-(4-(1-Carbamimidoyl-1,2,3,6-tetrahydropyridin-4-yl)-3-methyl phenyl)-1,3,4-oxadiazol-2-yl)phenyl)-5,6-dihydropyridine-1(2H)-carboximidamide;
4-(4-(5-(4-(1-Carbamimidoyl-1,2,3,6-tetrahydropyridin-4-yl)-2-methylphenyl)-1,3,4-oxadiazol-2-yl)phenyl)-5,6-dihydropyridine-1(2H)-carboximidamide;
4-(4-(5-(4-(1-Carbamimidoyl-1,2,3,6-tetrahydropyridin-4-yl)-2,3-dimethylphenyl)-1,3,4-oxadiazol-2-yl)phenyl)-5,6-dihydropyridine-1(2H)-carboximidamide;
4-(4-(5-(4-(1-Carbamimidoyl-1,2,3,6-tetrahydropyridin-4-yl)-2,6-dimethyl phenyl)-1,3,4-oxadiazol-2-yl)phenyl)-5,6-dihydropyridine-1(2H)-carboximidamide;
4-(4-(5-(4-(1-Carbamimidoyl-1,2,3,6-tetrahydropyridin-4-yl)-2-(trifluoromethyl)phenyl)-1,3,4-oxadiazol-2-yl)phenyl)-5,6-dihydropyridine-1(2H)-carboximidamide;
4-(4-(5-(4-(1-Carbamimidoyl-1,2,3,6-tetrahydropyridin-4-yl)-2-(methylamino)phenyl)-1,3,4-oxadiazol-2-yl)-3-fluorophenyl)-5,6-dihydropyridine-1(2H)-carboximidamide;
4,4′-(4,4′-(1,3,4-Oxadiazole-2,5-diyl)bis(3-(methylamino)-4,1-phenylene))bis(5,6-dihydropyridine-1(2H)-carboximidamide);
4,4′-(4,4′-(1,3,4-Oxadiazole-2,5-diyl)bis(3-fluoro-4,1-phenylene))bis(5,6-dihydropyridine-1(2H)-carboximidamide;
4,4′-(4,4′-(1,3,4-Oxadiazole-2,5-diyl)bis(2-fluoro-4,1-phenylene)) bis(5,6-dihydropyridine-1(2H)-carboximidamide);
4-(4-(5-(4-(1-Carbamimidoyl-1,2,3,6-tetrahydropyridin-4-yl)-2-fluorophenyl)-1,3,4-oxadiazol-2-yl)phenyl)-5,6-dihydropyridine-1(2H)-carboximidamide;
4,4′-(4,4′-(1,3,4-Oxadiazole-2,5-diyl)bis(4,1-phenylene))dipiperazine-1-carboximidamide;
4,4′-(4,4′-(1,3,4-Oxadiazole-2,5-diyl)bis(4,1-phenylene))dipiperidine-1-carboximidamide;
4-(4-(5-(4-(1-Carbamimidoylpiperidin-4-yl)-2-methylphenyl)-1,3,4-oxadiazol-2-yl)phenyl)piperidine-1-carboximidamide;
4,4′-(4,4′-(1-Methyl-1H-pyrazole-3,5-diyl)bis(4,1-phenylene)) bis(5,6-dihydropyridine-1(2H)-carboximidamide);
4,4′-(4,4′-(1-Methyl-1H-pyrazole-3,5-diyl)bis(4,1-phenylene))dipiperazine-1-carboximidamide;
4,4′-(4,4′-(4-Cyclohexyl-4H-1,2,4-triazole-3,5-diyl)bis(4,1-phenylene))bis(5,6-dihydropyridine-1(2H)-carboximidamide);
4,4′-(4,4′-(4-(2-Hydroxyethyl)-4H-1,2,4-triazole-3,5-diyl)bis(3-fluoro-4,1-phenylene))bis(5,6-dihydropyridine-1(2H)-carboximidamide);
4-(4-{5-[4-(1-carbamimidoylpiperidin-4-yl)phenyl]-1,3,4-oxadiazol-2-yl}-3-methylphenyl)piperidine-1-carboximidamide;
6-{5-[4-(1-carbamimidoyl-1,2,3,6-tetrahydropyridin-4-yl)phenyl]-1,3,4-oxadiazol-2-yl}-1′,2′,3′,6′-tetrahydro-[3,4′-bipyridine]-1′-carboximidamide;
4-(4-{5-[4-(4-Carbamimidoylpiperazin-1-yl)-3-methylphenyl]-1,3,4-oxadiazol-2-yl}phenyl)piperazine-1-carboximidamide;
4-(4-{5-[4-(4-Carbamimidoylpiperazin-1-yl)-2-methylphenyl]-1,3,4-oxadiazol-2-yl}phenyl)piperazine-1-carboximidamide;
4-(4-{5-[4-(1-Carbamimidoylpiperidin-4-yl)-2-methylphenyl]-1,3,4-oxadiazol-2-yl}phenyl)piperidine-1-carboximidamide;
4-(4-{5-[5-(4-Carbamimidoylpiperazin-1-yl)pyridin-2-yl]-1,3,4-oxadiazol-2-yl}phenyl)piperazine-1-carboximidamide;
4-(4-{4-[4-(1-Carbamimidoyl-1,2,3,6-tetrahydropyridin-4-yl)phenyl]-5-methyl-1H-1,2,3-triazol-1-yl}-2-methylphenyl)-1,2,3,6-tetrahydropyridine-1-carboximidamide;
4-(4-{4-[4-(1-Carbamimidoyl-1,2,3,6-tetrahydropyridin-4-yl)phenyl]-5-methyl-1H-1,2,3-triazol-1-yl}-2-methylphenyl)-1,2,3,6-tetrahydropyridine-1-carboximidamide;
4-(4-{5-[4-(1-carbamimidoyl-1,2,3,6-tetrahydropyridine-4-yl)-2-fluorophenyl]-4-(2-hydroxypropyl)-4H-1,2,4-triazol-3-yl}-3-fluorophenyl)-1,2,3,6-tetrahydropyridine-1-carboximidamide;
4-(4-{5-[4-(1-carbamimidoyl-1,2,3,6-tetrahydropyridin-4-yl)phenyl]-4-(2-methoxyethyl)-4H-1,2,4-triazol-3-yl}phenyl)-1,2,3,6-tetrahydropyridine-1-carboximidamide;
4-(4-{5-[4-(1-carbamimidoyl-1,2,3,6-tetrahydropyridin-4-yl)-2-fluorophenyl]-4-(2-methoxyethyl)-4H-1,2,4-triazol-3-yl}-3-fluorophenyl)-1,2,3,6-tetrahydropyridine-1-carboximidamide;
4-(4-{5-[4-(1-carbamimidoyl-1,2,3,6-tetrahydropyridin-4-yl)phenyl]-4-cyclopentyl-4H-1,2,4-triazol-3-yl}phenyl)-1,2,3,6-tetrahydropyridine-1-carboximidamide;
4-(4-{5-[4-(1-carbamimidoyl-1,2,3,6-tetrahydropyridin-4-yl)phenyl]-4-(2-hydroxyethyl)-4H-1,2,4-triazol-3-yl}phenyl)-1,2,3,6-tetrahydropyridine-1-carboximidamide;
4-(4-{5-[4-(1-carbamimidoyl-1,2,3,6-tetrahydropyridin-4-yl)-2-fluorophenyl]-4-(1,3-dihydroxypropan-2-yl)-4H-1,2,4-triazol-3-yl}-3-fluorophenyl)-1,2,3,6-tetrahydropyridine-1-carboximidamide;
4-(4-{5-[4-(1-carbamimidoyl-1,2,3,6-tetrahydropyridin-4-yl)-2-fluorophenyl]-4-[2 (dimethylamino)ethyl]-4H-1,2,4-triazol-3-yl}-3-fluorophenyl)-1,2,3,6-tetrahydropyridine-1-carboximidamide;
4-(4-{5-[4-(1-carbamimidoyl-1,2,3,6-tetrahydropyridin-4-yl)-2-fluorophenyl]-4-(2-hydroxy-2-methylpropyl)-4H-1,2,4-triazol-3-yl}-3-fluorophenyl)-1,2,3,6-tetrahydropyridine-1-carboximidamide;
4-(4-{5-[4-(1-carbamimidoyl-1,2,3,6-tetrahydropyridin-4-yl)-2-methoxyphenyl]-4-(2-hydroxyethyl)-4H-1,2,4-triazol-3-yl}-3-methoxyphenyl)-1,2,3,6-tetrahydropyridine-1-carboximidamide;
4-(4-{5-[4-(1-carbamimidoyl-1,2,3,6-tetrahydropyridin-4-yl)-3-methoxyphenyl]-4-(2-hydroxyethyl)-4H-1,2,4-triazol-3-yl}-2-methoxyphenyl)-1,2,3,6-tetrahydropyridine-1-carboximidamide;
4-(4-{5-[4-(1-carbamimidoyl-1,2,3,6-tetrahydropyridin-4-yl)-2-fluorophenyl]-4-[2-(2-hydroxyethoxy)ethyl]-4H-1,2,4-triazol-3-yl}-3-fluorophenyl)-1,2,3,6-tetrahydropyridine-1-carboximidamide;
4-(4-{5-[4-(1-carbamimidoyl-1,2,3,6-tetrahydropyridin-4-yl)-2-fluorophenyl]-4-methyl-4H-1,2,4-triazol-3-yl}phenyl)-1,2,3,6-tetrahydropyridine-1-carboximidamide;
4-(4-{5-[4-(1-carbamimidoyl-1,2,3,6-tetrahydropyridin-4-yl)-2-fluorophenyl]-4-methyl-4H-1,2,4-triazol-3-yl}-2-fluorophenyl)-1,2,3,6-tetrahydropyridine-1-carboximidamide;
4-(4-{5-[4-(1-carbamimidoyl-1,2,3,6-tetrahydropyridin-4-yl)-3-(trifluoromethyl)phenyl]-4-(2-hydroxyethyl)-4H-1,2,4-triazol-3-yl}-2-(trifluoromethyl)phenyl)-1,2,3,6-tetrahydropyridine-1-carboximidamide;
4-(4-{5-[4-(1-carbamimidoyl-1,2,3,6-tetrahydropyridin-4-yl)-2-fluorophenyl]-4-[2-(2-methoxyethoxy)ethyl]-4H-1,2,4-triazol-3-yl}-3-fluorophenyl)-1,2,3,6-tetrahydropyridine-1-carboximidamide;
4-(4-{5-[4-(1-carbamimidoyl-1,2,3,6-tetrahydropyridin-4-yl)-2-fluorophenyl]-4-(2-hydroxyethyl)-4H-1,2,4-triazol-3-yl}phenyl)-1,2,3,6-tetrahydropyridine-1-carboximidamide;
4-(4-{5-[4-(1-carbamimidoyl-1,2,3,6-tetrahydropyridin-4-yl)-2-fluorophenyl]-4-(1-hydroxypropan-2-yl)-4H-1,2,4-triazol-3-yl}-3-fluorophenyl)-1,2,3,6-tetrahydropyridine-1-carboximidamide;
4-(4-{5-[4-(1-carbamimidoyl-1,2,3,6-tetrahydropyridin-4-yl)-2-fluorophenyl]-4-(2,2,2-trifluoroethyl)-4H-1,2,4-triazol-3-yl}-3-fluorophenyl)-1,2,3,6-tetrahydropyridine-1-carboximidamide;
4-(4-{5-[4-(1-carbamimidoyl-1,2,3,6-tetrahydropyridin-4-yl)-2-fluorophenyl]-4-methyl-4H-1,2,4-triazol-3-yl}-2,6-difluorophenyl)-1,2,3,6-tetrahydropyridine-1-carboximidamide;
4-(4-{5-[4-(4-carbamimidoylcyclohex-1-en-1-yl)-2-fluorophenyl]-4-(2-methoxyethyl)-4H-1,2,4-triazol-3-yl}-3-fluorophenyl)cyclohex-3-ene-1-carboximidamide;
4-(4-{5-[4-(4-carbamimidoylcyclohex-1-en-1-yl)-2-fluorophenyl]-4-methyl-4H-1,2,4-triazol-3-yl}-3-fluorophenyl)cyclohex-3-ene-1-carboximidamide;
or a pharmaceutically acceptable form thereof.

56. A composition comprising an effective amount of at least one compound according to claim 44.

57. A composition according to claim 56, further comprising at least one excipient.

58. A composition according to claim 57, wherein the at least one compound is at least one member selected from the group consisting of:
4,4′-(4,4′-(4-Methyl-4H-1,2,4-triazole-3,5-diyl)bis(4,1-phenylene))bis(5,6-dihydropyridine-1(2H)-carboximidamide);
4,4′-(4,4′-(4-Ethyl-4H-1,2,4-triazole-3,5-diyl)bis(4,1-phenylene))bis(5,6-dihydropyridine-1(2H)-carboximidamide);
4,4′-(4,4′-(4-Isopropyl-4H-1,2,4-triazole-3,5-diyl)bis(4,1-phenylene))bis(5,6-dihydropyridine-1(2H)-carboximidamide);
4,4′-(4,4′-(4-Cyclopropyl-4H-1,2,4-triazole-3,5-diyl)bis(4,1-phenylene)) bis(5,6-dihydropyridine-1(2H)-carboximidamide);
4,4′-(4,4′-(4-Benzyl-4H-1,2,4-triazole-3,5-diyl)bis(4,1-phenylene))bis(5,6-dihydropyridine-1(2H)-carboximidamide);
4-(4-(4-Benzyl-5-(4-(1-carbamimidoyl-1,2,3,6-tetrahydropyridin-4-yl)-2-fluorophenyl)-4H-1,2,4-triazol-3-yl)phenyl)-5,6-dihydropyridine-1(2H)-carboximidamide;
4,4′-(4,4′-(4-Methyl-4H-1,2,4-triazole-3,5-diyl)bis(3-fluoro-4,1-phenylene)) bis(5,6-dihydropyridine-1(2H)-carboximidamide);
4,4′-(4,4′-(4-(2-(2-Ethoxyethoxy)ethyl)-4H-1,2,4-triazole-3,5-diyl)bis(3-fluoro-4,1-phenylene))bis(5,6-dihydropyridine-1(2H)-carboximidamide);
4,4′-(4,4′-(4-Ethyl-4H-1,2,4-triazole-3,5-diyl)bis(3-fluoro-4,1-phenylene)) bis(5,6-dihydropyridine-1(2H)-carboximidamide)′
4,4′-(4,4′-(4-Octyl-4H-1,2,4-triazole-3,5-diyl)bis(3-fluoro-4,1-phenylene))bis(5,6-dihydropyridine-1(2H)-carboximidamide);
4,4′-(4,4′-(4-(1-Carbamimidoylpiperidin-4-yl)-4H-1,2,4-triazole-3,5-diyl)bis (3-fluoro-4,1-phenylene))bis(5,6-dihydropyridine-1(2H)-carboximidamide);
4,4′-(4,4′-(4-Isopropyl-4H-1,2,4-triazole-3,5-diyl)bis(3-fluoro-4,1-phenylene))bis(5,6-dihydropyridine-1(2H)-carboximidamide);
4,4′-(4,4′-(4-Isobutyl-4H-1,2,4-triazole-3,5-diyl)bis(3-fluoro-4,1-phenylene)) bis(5,6-dihydropyridine-1(2H)-carboximidamide));
4,4′-(4,4′-(4-(1-Methylpiperidin-4-yl)-4H-1,2,4-triazole-3,5-diyl)bis(3-fluoro-4,1-phenylene))bis(5,6-dihydropyridine-1(2H)-carboximidamide);
4,4′-(4,4′-(4-(2-guanidinoethyl)-4H-1,2,4-triazole-3,5-diyl)bis(3-fluoro-4,1-phenylene))bis(5,6-dihydropyridine-1(2H)-carboximidamide);
4,4′-(4,4′-(4-Cyclopropyl-4H-1,2,4-triazole-3,5-diyl)bis(3-fluoro-4,1-phenylene))bis (5,6-dihydropyridine-1 (2H)-carboximidamide);
4,4′-(4,4′-(4-Benzyl-4H-1,2,4-triazole-3,5-diyl)bis(3-fluoro-4,1-phenylene)) bis(5,6-dihydropyridine-1(2H)-carboximidamide;
4,4′-(4,4′-(4-Methyl-4H-1,2,4-triazole-3,5-diyl)bis(2-fluoro-4,1-phenylene)) bis(5,6-dihydropyridine-1(2H)-carboximidamide);
4,4′-(4,4′-(4-Methyl-4H-1,2,4-triazole-3,5-diyl)bis(2,5-difluoro-4,1-phenylene))bis(5,6-dihydropyridine-1(2H)-carboximidamide);
4-(4-(5-(4-(1-Carbamimidoyl-1,2,3,6-tetrahydropyridin-4-yl)-2-methylphenyl)-4-methyl-4H-1,2,4-triazol-3-yl)phenyl)-5,6-dihydropyridine-1(2H)-carboximidamide;
4,4′-(4,4′-(4-Methyl-4H-1,2,4-triazole-3,5-diyl)bis(3-methyl-4,1-phenylene))bis(5,6-dihydropyridine-1(2H)-carboximidamide);
4-(4-(5-(4-(1-Carbamimidoyl-1,2,3,6-tetrahydropyridin-4-yl)-2-fluoro-6-(methylamino)phenyl)-4-methyl-4H-1,2,4-triazol-3-yl)phenyl)-5,6-dihydropyridine-1(2H)-carboximidamide;
4,4′-(4,4′-(4-Methyl-4H-1,2,4-triazole-3,5-diyl)bis(2-methyl-4,1-phenylene)) bis(5,6-dihydropyridine-1(2H)-carboximidamide);
4-(4-(5-(4-(1-Carbamimidoyl-1,2,3,6-tetrahydropyridin-4-yl)-3-(trifluoromethyl)phenyl)-4-methyl-4H-1,2,4-triazol-3-yl)phenyl)-5,6-dihydropyridine-1(2H)-carboximidamide;
4-(4-(5-(4-(1-Carbamimidoyl-1,2,3,6-tetrahydropyridin-4-yl)-2-(isopropylamino)phenyl)-4-isopropyl-4H-1,2,4-triazol-3-yl)-3-fluorophenyl)-5,6-dihydro pyridine-1(2H)-carboximidamide;
3-(4-(5-(4-(1-Carbamimidoyl-2,5-dihydro-1H-pyrrol-3-yl)-3-(trifluoromethyl)phenyl)-4-methyl-4H-1,2,4-triazol-3-yl)phenyl)-2,5-dihydro-1H-pyrrole-1-carboximidamide;
3,3′-(4,4′-(4-Methyl-4H-1,2,4-triazole-3,5-diyl)bis(3-fluoro-4,1-phenylene)) bis(2,5-dihydro-1H-pyrrole-1-carboximidamide);
3,3′-(4,4′-(4-Methyl-4H-1,2,4-triazole-3,5-diyl)bis(4,1-phenylene))bis(2,5-dihydro-1H-pyrrole-1-carboximidamide;
4-(4-{1-[4-(1-Carbamimidoyl-1,2,3,6-tetrahydropyridin-4-yl) phenyl]-1H-1,2,3-triazol-4-yl}phenyl)-1,2,3,6-tetrahydropyridine-1-carboximidamide;
4-(4-{1-[4-(1-Carbamimidoyl-1,2,3,6-tetrahydropyridin-4-yl)phenyl]-5-methyl-1H-1,2,3-triazol-4-yl}phenyl)-1,2,3,6-tetrahydropyridine-1-carboximidamide;
4-(4-(1-(4-(1-Carbamimidoyl-1,2,3,6-tetrahydropyridin-4-yl)-2-methyl phenyl)-5-methyl-1H-1,2,3-triazol-4-yl)phenyl)-5,6-dihydropyridine-1(2H)-carboximidamide;
4-(4-(1-(4-(1-Carbamimidoyl-1,2,3,6-tetrahydropyridin-4-yl)-3-methylphenyl)-5-methyl-1H-1,2,3-triazol-4-yl)phenyl)-5,6-dihydropyridine-1(2H)-carboximidamide;
4-(4-(1-(4-(1-Carbamimidoyl-1,2,3,6-tetrahydropyridin-4-yl)-2-methylphenyl)-1H-1,2,3-triazol-4-yl)phenyl)-5,6-dihydropyridine-1(2H)-carboximidamide;
4-(4-(1-(4-(1-Carbamimidoyl-1,2,3,6-tetrahydropyridin-4-yl)-3-methyl phenyl)-1H-1,2,3-triazol-4-yl)phenyl)-5,6-dihydropyridine-1(2H)-carboximidamide;
4-(4-(4-(4-(1-Carbamimidoyl-1,2,3,6-tetrahydropyridin-4-yl)-2-methylphenyl)-1H-1,2,3-triazol-1-yl)phenyl)-5,6-dihydropyridine-1(2H)-carboximidamide;
4-(4-(4-(4-(1-Carbamimidoyl-1,2,3,6-tetrahydropyridin-4-yl)-3-methyl phenyl)-1H-1,2,3-triazol-1-yl)phenyl)-5,6-dihydropyridine-1(2H)-carboximidamide;
4,4′-(4,4′-(1-Methyl-1H-1,2,4-triazole-3,5-diyl)bis(4,1-phenylene)) bis(5,6-dihydropyridine-1(2H)-carboximidamide);
4,4′-(4,4′-(4-Methyl-4H-1,2,4-triazole-3,5-diyl)bis(4,1-phenylene))dipiperidine-1-carboximidamide;
1-[4-(4-{5-[4-(4-Carbamimidamidocyclohex-1-en-1-yl)-2-fluorophenyl]-4-methyl-4H-1,2,4-triazol-3-yl}-3-fluorophenyl)cyclohex-3-en-1-yl]guanidine;
4-(4-{5-[4-(1-Carbamimidoyl-1,2,3,6-tetrahydropyridin-4-yl)phenyl]-1,3,4-oxadiazol-2-yl}phenyl)-1,2,3,6-tetrahydropyridine-1-carboximidamide;
4-(4-(5-(4-(1-Carbamimidoyl-1,2,3,6-tetrahydropyridin-4-yl)-3-methyl phenyl)-1,3,4-oxadiazol-2-yl)phenyl)-5,6-dihydropyridine-1(2H)-carboximidamide;
4-(4-(5-(4-(1-Carbamimidoyl-1,2,3,6-tetrahydropyridin-4-yl)-2-methylphenyl)-1,3,4-oxadiazol-2-yl)phenyl)-5,6-dihydropyridine-1(2H)-carboximidamide;
4-(4-(5-(4-(1-Carbamimidoyl-1,2,3,6-tetrahydropyridin-4-yl)-2,3-dimethylphenyl)-1,3,4-oxadiazol-2-yl)phenyl)-5,6-dihydropyridine-1(2H)-carboximidamide;
4-(4-(5-(4-(1-Carbamimidoyl-1,2,3,6-tetrahydropyridin-4-yl)-2,6-dimethyl phenyl)-1,3,4-oxadiazol-2-yl)phenyl)-5,6-dihydropyridine-1(2H)-carboximidamide;
4-(4-(5-(4-(1-Carbamimidoyl-1,2,3,6-tetrahydropyridin-4-yl)-2-(trifluoromethyl)phenyl)-1,3,4-oxadiazol-2-yl)phenyl)-5,6-dihydropyridine-1(2H)-carboximidamide;
4-(4-(5-(4-(1-Carbamimidoyl-1,2,3,6-tetrahydropyridin-4-yl)-2-(methylamino)phenyl)-1,3,4-oxadiazol-2-yl)-3-fluorophenyl)-5,6-dihydropyridine-1(2H)-carboximidamide;
4,4′-(4,4′-(1,3,4-Oxadiazole-2,5-diyl)bis(3-(methylamino)-4,1-phenylene))bis(5,6-dihydropyridine-1(2H)-carboximidamide);
4,4′-(4,4′-(1,3,4-Oxadiazole-2,5-diyl)bis(3-fluoro-4,1-phenylene))bis(5,6-dihydropyridine-1(2H)-carboximidamide;
4,4′-(4,4′-(1,3,4-oxadiazole-2,5-diyl)bis(2-fluoro-4,1-phenylene)) bis(5,6-dihydropyridine-1(2H)-carboximidamide);
4-(4-(5-(4-(1-Carbamimidoyl-1,2,3,6-tetrahydropyridin-4-yl)-2-fluorophenyl)-1,3,4-oxadiazol-2-yl)phenyl)-5,6-dihydropyridine-1(2H)-carboximidamide;
4,4′-(4,4′-(1,3,4-Oxadiazole-2,5-diyl)bis(4,1-phenylene))dipiperazine-1-carboximidamide;
4,4′-(4,4′-(1,3,4-Oxadiazole-2,5-diyl)bis(4,1-phenylene))dipiperidine-1-carboximidamide;
4-(4-(5-(4-(1-Carbamimidoylpiperidin-4-yl)-2-methylphenyl)-1,3,4-oxadiazol-2-yl)phenyl)piperidine-1-carboximidamide;
4,4′-(4,4′-(1-Methyl-1H-pyrazole-3,5-diyl)bis(4,1-phenylene))bis(5,6-dihydropyridine-1(2H)-carboximidamide);
4,4′-(4,4′-(1-Methyl-1H-pyrazole-3,5-diyl)bis(4,1-phenylene))dipiperazine-1-carboximidamide;
4,4′-(4,4′-(4-Cyclohexyl-4H-1,2,4-triazole-3,5-diyl)bis(4,1-phenylene))bis(5,6-dihydropyridine-1(2H)-carboximidamide);
4,4′-(4,4′-(4-(2-Hydroxyethyl)-4H-1,2,4-triazole-3,5-diyl)bis(3-fluoro-4,1-phenylene))bis(5,6-dihydropyridine-1(2H)-carboximidamide);
4-(4-{5-[4-(1-Carbamimidoylpiperidin-4-yl)phenyl]-1,3,4-oxadiazol-2-yl}-3-methylphenyl)piperidine-1-carboximidamide;
6-{5-[4-(1-Carbamimidoyl-1,2,3,6-tetrahydropyridin-4-yl)phenyl]-1,3,4-oxadiazol-2-yl}-1′,2′,3′,6′-tetrahydro-[3,4′-bipyridine]-1′-carboximidamide;
4-(4-{5-[4-(4-Carbamimidoylpiperazin-1-yl)-3-methylphenyl]-1,3,4-oxadiazol-2-yl}phenyl)piperazine-1-carboximidamide;
4-(4-{5-[4-(4-Carbamimidoylpiperazin-1-yl)-2-methylphenyl]-1,3,4-oxadiazol-2-yl}phenyl)piperazine-1-carboximidamide;
4-(4-{5-[4-(1-Carbamimidoylpiperidin-4-yl)-2-methylphenyl]-1,3,4-oxadiazol-2-yl}phenyl)piperidine-1-carboximidamide;
4-(4-{5-[5-(4-Carbamimidoylpiperazin-1-yl)pyridin-2-yl]-1,3,4-oxadiazol-2-yl}phenyl)piperazine-1-carboximidamide;
4-(4-{4-[4-(1-Carbamimidoyl-1,2,3,6-tetrahydropyridin-4-yl)phenyl]-5-methyl-1H-1,2,3-triazol-1-yl}-2-methylphenyl)-1,2,3,6-tetrahydropyridine-1-carboximidamide;
4-(4-{4-[4-(1-Carbamimidoyl-1,2,3,6-tetrahydropyridin-4-yl)phenyl]-5-methyl-1H-1,2,3-triazol-1-yl}-2-methylphenyl)-1,2,3,6-tetrahydropyridine-1-carboximidamide;
4-(4-{5-[4-(1-carbamimidoyl-1,2,3,6-tetrahydropyridine-4-yl)-2-fluorophenyl]-4-(2-hydroxypropyl)-4H-1,2,4-triazol-3-yl}-3-fluorophenyl)-1,2,3,6-tetrahydropyridine-1-carboximidamide;
4-(4-{5-[4-(1-carbamimidoyl-1,2,3,6-tetrahydropyridin-4-yl)phenyl]-4-(2-methoxyethyl)-4H-1,2,4-triazol-3-yl}phenyl)-1,2,3,6-tetrahydropyridine-1-carboximidamide;
4-(4-{5-[4-(1-carbamimidoyl-1,2,3,6-tetrahydropyridin-4-yl)-2-fluorophenyl]-4-(2-methoxyethyl)-4H-1,2,4-triazol-3-yl}-3-fluorophenyl)-1,2,3,6-tetrahydropyridine-1-carboximidamide;
4-(4-{5-[4-(1-carbamimidoyl-1,2,3,6-tetrahydropyridin-4-yl)phenyl]-4-cyclopentyl-4H-1,2,4-triazol-3-yl}phenyl)-1,2,3,6-tetrahydropyridine-1-carboximidamide;
4-(4-{5-[4-(1-carbamimidoyl-1,2,3,6-tetrahydropyridin-4-yl)phenyl]-4-(2-hydroxyethyl)-4H-1,2,4-triazol-3-yl}phenyl)-1,2,3,6-tetrahydropyridine-1-carboximidamide;
4-(4-{5-[4-(1-carbamimidoyl-1,2,3,6-tetrahydropyridin-4-yl)-2-fluorophenyl]-4-(1,3-dihydroxypropan-2-yl)-4H-1,2,4-triazol-3-yl}-3-fluorophenyl)-1,2,3,6-tetrahydropyridine-1-carboximidamide;
4-(4-{5-[4-(1-carbamimidoyl-1,2,3,6-tetrahydropyridin-4-yl)-2-fluorophenyl]-4-[2 (dimethylamino)ethyl]-4H-1,2,4-triazol-3-yl}-3-fluorophenyl)-1,2,3,6-tetrahydropyridine-1-carboximidamide;
4-(4-{5-[4-(1-carbamimidoyl-1,2,3,6-tetrahydropyridin-4-yl)-2-fluorophenyl]-4-(2-hydroxy-2-methylpropyl)-4H-1,2,4-triazol-3-yl}-3-fluorophenyl)-1,2,3,6-tetrahydropyridine-1-carboximidamide;
4-(4-{5-[4-(1-carbamimidoyl-1,2,3,6-tetrahydropyridin-4-yl)-2-methoxyphenyl]-4-(2-hydroxyethyl)-4H-1,2,4-triazol-3-yl}-3-methoxyphenyl)-1,2,3,6-tetrahydropyridine-1-carboximidamide;
4-(4-{5-[4-(1-carbamimidoyl-1,2,3,6-tetrahydropyridin-4-yl)-3-methoxyphenyl]-4-(2-hydroxyethyl)-4H-1,2,4-triazol-3-yl}-2-methoxyphenyl)-1,2,3,6-tetrahydropyridine-1-carboximidamide;
4-(4-{5-[4-(1-carbamimidoyl-1,2,3,6-tetrahydropyridin-4-yl)-2-fluorophenyl]-4-[2-(2-hydroxyethoxy)ethyl]-4H-1,2,4-triazol-3-yl}-3-fluorophenyl)-1,2,3,6-tetrahydropyridine-1-carboximidamide;
4-(4-{5-[4-(1-carbamimidoyl-1,2,3,6-tetrahydropyridin-4-yl)-2-fluorophenyl]-4-methyl-4H-1,2,4-triazol-3-yl}phenyl)-1,2,3,6-tetrahydropyridine-1-carboximidamide;
4-(4-{5-[4-(1-carbamimidoyl-1,2,3,6-tetrahydropyridin-4-yl)-2-fluorophenyl]-4-methyl-4H-1,2,4-triazol-3-yl}-2-fluorophenyl)-1,2,3,6-tetrahydropyridine-1-carboximidamide;
4-(4-{5-[4-(1-carbamimidoyl-1,2,3,6-tetrahydropyridin-4-yl)-3-(trifluoromethyl)phenyl]-4-(2-hydroxyethyl)-4H-1,2,4-triazol-3-yl}-2-(trifluoromethyl)phenyl)-1,2,3,6-tetrahydropyridine-1-carboximidamide;
4-(4-{5-[4-(1-carbamimidoyl-1,2,3,6-tetrahydropyridin-4-yl)-2-fluorophenyl]-4-[2-(2-methoxyethoxy)ethyl]-4H-1,2,4-triazol-3-yl}-3-fluorophenyl)-1,2,3,6-tetrahydropyridine-1-carboximidamide;
4-(4-{5-[4-(1-carbamimidoyl-1,2,3,6-tetrahydropyridin-4-yl)-2-fluorophenyl]-4-(2-hydroxyethyl)-4H-1,2,4-triazol-3-yl}phenyl)-1,2,3,6-tetrahydropyridine-1-carboximidamide;
4-(4-{5-[4-(1-carbamimidoyl-1,2,3,6-tetrahydropyridin-4-yl)-2-fluorophenyl]-4-(1-hydroxypropan-2-yl)-4H-1,2,4-triazol-3-yl}-3-fluorophenyl)-1,2,3,6-tetrahydropyridine-1-carboximidamide;
4-(4-{5-[4-(1-carbamimidoyl-1,2,3,6-tetrahydropyridin-4-yl)-2-fluorophenyl]-4-(2,2,2-trifluoroethyl)-4H-1,2,4-triazol-3-yl}-3-fluorophenyl)-1,2,3,6-tetrahydropyridine-1-carboximidamide;
4-(4-{5-[4-(1-carbamimidoyl-1,2,3,6-tetrahydropyridin-4-yl)-2-fluorophenyl]-4-methyl-4H-1,2,4-triazol-3-yl}-2,6-difluorophenyl)-1,2,3,6-tetrahydropyridine-1-carboximidamide;
4-(4-{5-[4-(4-carbamimidoylcyclohex-1-en-1-yl)-2-fluorophenyl]-4-(2-methoxyethyl)-4H-1,2,4-triazol-3-yl}-3-fluorophenyl)cyclohex-3-ene-1-carboximidamide;
4-(4-{5-[4-(4-carbamimidoylcyclohex-1-en-1-yl)-2-fluorophenyl]-4-methyl-4H-1,2,4-triazol-3-yl}-3-fluorophenyl)cyclohex-3-ene-1-carboximidamide;
or a pharmaceutically acceptable form thereof.

59. A method of treating or preventing disease or conditions associated with fungal infection wherein said methods comprises administering to a subject an effective amount of at least one compound according to claim 44.

60. The method of claim 59, wherein the at least one compound is administered in a composition further comprising at least one excipient.

61. The method of claim 59 wherein the fungal infection is an organism from a genus selected from the group consisting of Candida, Cryptococcus, Trichosporon, Malassezia, Aspergillus, Fusarium, Mucor, Blastomyces, Coccidioides, Pneumocystis, Histoplasma, Trichophyton, Rhizopus, Apophysomyces, Rhizomucor, Lichtheimia, Scedosporium, and Lomentospora.

62. The method of claim 61, wherein the at least one compound is administered in a composition further comprising at least one excipient.

63. The method of claim 59 wherein the fungal infection is an organism selected from the group consisting of Candida albicans, Candida glabrata, Candida krusei, Candida tropicalis, Candida guilliermondii, Candida parapsilosis, Candida dubliniensis Candida auris, Cryptococcus neoformans, Cryptococcus gatti, Trichosporon asahii, Trichosporon asteroides, Trichosporon cutaneum, Trichosporon dermatis, Trichosporon dohaense, Trichosporon inkin, Trichosporon loubieri, Trichosporon mucoides, Trichosporon ovoides, Malassezia globose, Malassezia restricta, Aspergillus fumigatus. Aspergillus. flavis, Aspergillus terreus, Aspergillus niger, Fusarium solani, Fusarium falciforme, Fusarium oxysporum, Fusarium verticillioides, Fusarium proliferatum, Mucor circinelloides, Mucor ramosissimus, Mucor indicus, Mucor rasemosus, Mucor piriformis, Blastomyces dermatitidis, Blastomyces brasiliensis, Coccidioides immitis, Coccidioides posadasii, Pneumocystis carinii, Pneumocystis jiroveci, Histoplasma capsulatum, Trichophyton schoenleinii, Trichophyton mentagrophytes, Trichophyton verrucosum, Trichophyton rubrum, Rhizopus oryzae, Rhizopus stolonifera, Apophysomyces variabilis, Rhizomucor pusillus, Rhizomucor regularior, Rhizomucor chlamydosporus, Lichtheimia ramosa, Lichtheimia corymbifera, Scedosporium apiospermum, and Lomentospora prolificans.

64. The method of claim 63, wherein the at least one compound is administered in a composition further comprising at least one excipient.

65. The method of treating or preventing disease or conditions associated with fungal infection, including candidemia, oral candidiasis, vulvovaginal candidiasis, aspergillosis, allergic bronchopulmonary aspergillosis, allergic Aspergillus sinusitis, invasive aspergillosis, disseminated aspergillosis, cryptococcosis, pulmonary cryptococcosis, meningeal cryptococcosis, skin keratitis, athlete's foot, ringworm, ocular keratitis, onychomycosis, sinusitis, endophthalmitis, otitis, endocarditism pneumonia, osteomyelitis, meningitis, ventriculitis, COVID-19 Associated Pulmonary Aspergillosis (CAPA) and Influenza Associated Pulmonary Aspergillosis (IAPA) wherein said method comprises administering to a subject an effective amount of at least one compound according to claim 44.

66. The method of claim 65, wherein the at least one compound is administered in a composition further comprising at least one excipient.

67. The method of treating or preventing fungal infection in plants including wilt disease in tomato, wilt disease cotton, wilt disease banana, wilt of gram, downy mildew of cereals, damping of seedling, rot of ginger, late blight of potato, early blight of potato, blast disease of rice, powdery mildews, tikka disease of groundnut, leaf rust of coffee, red rot of sugarcane, brown rot in pear, brown rot in plum, brown rot in peach, leaf spot of oats, black wart disease of potato, yellow rust of wheat, white rust of crucifers, maize smut, loose smut of wheat, flag smut of wheat, covered smut of barley, black rust of wheat, bankanese disease, foot rot of rice, and ergot disease of rye wherein said methods comprise administering to a plant an effective amount of a compound according to claim 44.

68. The method of claim 67, wherein the at least one compound is administered in a composition further comprising at least one excipient.

69. The method of treating or preventing fungal infections in domesticated animals, livestock, and companion animals including candidiasis infections in animals selected from the group consisting of cattle, sheep, pigs, goats, horses, donkeys, mules, buffalo, oxen, llamas, camels, dogs, cats, horses, rabbits, ferrets, and guinea pigs, wherein said methods comprise administering to a plant an effective amount of a compound according to claim 44.

70. The method of claim 69, wherein the at least one compound is administered in a composition further comprising at least one excipient.

71. The method of treating or preventing aspergillosis infections in horses, cattle, sheep, goats, dogs and cats wherein said methods comprise administering to a plant an effective amount of a compound according to claim 44.

72. The method of claim 71, wherein the at least one compound is administered in a composition further comprising at least one excipient.

73. The method of treating or preventing mucormycosis infections in horses, cattle, sheep, goats, dogs and cats wherein said methods comprise administering to a plant an effective amount of a compound according to claim 44.

74. The method of claim 73, wherein the at least one compound is administered in a composition further comprising at least one excipient.

75. The method of treating or preventing coccidioidomycosis in dogs and cats caused by infection with an organism selected from the group consisting of Coccidioides immitis and Coccidioides posadasii, wherein said methods comprise administering to a plant an effective amount of a compound according to claim 44.

76. The method of claim 75, wherein the at least one compound is administered in a composition further comprising at least one excipient.

77. The method of treating or preventing blastomycosis in dogs and cats caused by infection with Blastomyces dermatitidis, wherein said methods comprise administering to a plant an effective amount of a compound according to claim 44.

78. The method of claim 77, wherein the at least one compound is administered in a composition further comprising at least one excipient.

79. The method of treating or preventing Paracoccidioidomycosis in dogs caused by infection with Paracoccidioides brasiliensis, wherein said methods comprise administering to a plant an effective amount of a compound according to claim 44.

80. The method of claim 79, wherein the at least one compound is administered in a composition further comprising at least one excipient.

81. The method of treating or preventing dermatophytosis (ringworm) in cats and dogs caused by infection with an organism selected from the group consisting of Microsporum canis, Microsporum gypseum, and Trichophyton mentagrophytes, wherein said methods comprise administering to a plant an effective amount of a compound according to claim 44.

82. The method of claim 81, wherein the at least one compound is administered in a composition further comprising at least one excipient.

83. The method of treating or preventing cryptococcosis in dogs and cats caused by infection with an organism selected from the group consisting of Cryptococcus neoformans and Cryptococcus gattii, wherein said methods comprise administering to a plant an effective amount of a compound according to claim 44.

84. The method of claim 83, wherein the at least one compound is administered in a composition further comprising at least one excipient.

85. The method of treating or preventing histoplasmosis in dogs caused by infection with Histoplasma capsulatum, wherein said methods comprise administering to a plant an effective amount of a compound according to claim 44.

86. The method of claim 83, wherein the at least one compound is administered in a composition further comprising at least one excipient.
